(19) United States
(12) Patent Application Publication
(10) Pub. No.: US 2026/0138990 A1
(43) Pub. Date: May 21, 2026

(54) TRICYCLIC FUSED IMIDAZOLE COMPOUNDS AS CD38 MODULATORS AND USES THEREOF

(71) Applicant: AEOVIAN PHARMACEUTICALS, INC., Berkeley, CA (US)

(72) Inventors: John KINCAID, Fox Point, WI (US); Anthony Kyle BALL, Nottingham (GB)

(21) Appl. No.: 19/366,111

(22) Filed: Oct. 22, 2025

Related U.S. Application Data
(63) Continuation of application No. PCT/US2024/026107, filed on Apr. 24, 2024.
(60) Provisional application No. 63/498,182, filed on Apr. 25, 2023.
(60) Provisional application No. 63/578,614, filed on Aug. 24, 2023.
(60) Provisional application No. 63/627,213, filed on Jan. 31, 2024.

(51) Int. Cl. C07D 498/04; A61K 31/4188; A61K 31/4985; A61K 31/553; C07D 487/04; C07D 498/14; C07D 498/18; C07D 519/00

(52) U.S. Cl. CPC C07D 498/04; A61K 31/4188; A61K 31/4985; A61K 31/553; C07D 487/04; C07D 498/14; C07D 498/18; C07D 519/00

(57) ABSTRACT

The disclosure provides compounds and salts for the treatment of disease.

### CROSS-REFERENCE

This application is a continuation of International Patent Application No. PCT/US2024/026107 filed on Apr. 24, 2024, which claims the benefit of U.S. Provisional Patent Application No. 63/498,182 filed on Apr. 25, 2023, U.S. Provisional Patent Application No. 63/578,614 filed on Aug. 24, 2023, and U.S. Provisional Patent Application No. 63/627,213 filed on Jan. 31, 2024, each of which is incorporated herein by reference in its entirety.

### BACKGROUND OF THE INVENTION

Nicotinamide Adenine Dinucleotide (NAD) is a biochemical found in all cells that was first characterized over 100 years ago due to its role in oxidoreductase reactions. Since then. NAD⁺ and its related pyridine nucleotides NADH, NADP⁺, and NADPH are recognized as the major redox carriers in all organisms. These pyridine dinucleotides regulate the cytosolic and mitochondrial redox state and are key participants monitoring the metabolic status of the cell. This is because NAD and NADH act as hydride accepting and donating cofactors for metabolic enzymes involved in glycolysis, the TCA cycle, and the respiratory chain and thereby redistribute reducing equivalents generated from these catabolic processes into the de novo synthesis of new biomolecules. (Houtkooper et al Endo Reviews (2010) 31:194-223; Koch-Nolte et al Science Signaling (2009) 2: mr1: Houtkooper and Auwerx J. Cell Biol (2012) 199:205-209; Berger et al Trends in Bioch Sci (2004) 29:111-18).

In addition to its long recognized role as a cofactor for oxidoreductases, more recent research demonstrates that NAD⁺ is also a substrate for various enzymes, where it is consumed in the process of donating its ADP ribose to acceptor molecules. The enzymes that are the major consumers of NAD⁺ are the ADP ribosyl transferases (i.e., PARP and ART family of enzymes), the sirtuins (Sirt1-7), and the ADP ribosyl cyclases/hydrolases (CD38/CD157). These enzymes are involved in pathways that regulate Ca⁺⁺ signaling, gene transcription, DNA repair, cell survival, energy metabolism, and oxidative stress. Thus, NAD⁺ and its phosphorylated relatives NADP and NAADP, both of which are derived from NAD⁺, also act as signaling molecules. NAD⁺ is also a key component of the circadian cycle with daily oscillations that tie cellular metabolism to chromatin remodeling and gene transcription.

It is known that exercise and caloric restriction elevate NAD⁺ levels, while aging and obesity decrease cellular NAD⁺ levels. Restoring NAD⁺ levels in disease states that consume significant amounts of NAD⁺ will likely have medical benefits as the cell strives to maintain its energy status during stress. (Tevy et al Trends in Endo and Metab (2013) 24:229-237; Pugh et al Aging Cell (2013) 12:672-681; Massudi et al PLOS ONE (2012) 7: e42357; Xu and Sauve (2010) Mech of Ageing and Development 131:287-298). Cellular NAD⁺ is produced by either the de novo synthesis pathway from tryptophan or by a salvage synthesis pathway from precursors such as nicotinic acid (niacin) and nicotinamide, both of which are obtained from dietary sources.

A third way to modulate cellular NAD⁺ levels is to block consumption of NAD⁺ by inhibiting enzymes that consume NAD⁺. Cluster of differentiation 38 (CD38) is one such consumer of NAD⁺. Also known as ADP ribosyl cyclase, CD38 is a type II membrane-anchored enzyme. It efficiently catalyzes the breakdown of NAD⁺ to nicotinamide and ADPR and hydrolyzes NAADP to ADPRP. CD38 can also act as a cyclase converting NAD⁺ to cADPR, although it is 100-fold less efficient as a cyclase than as a hydrolase.

CD38 was first characterized as a surface antigen on immune cells and is broadly distributed throughout most tissues in the body. It exists on the plasma membrane and on the membranes of intracellular organelles such as the nucleus and mitochondria. As predicted from its function as a NAD⁺ glycohydrolase, CD38 KO mice have elevated NAD⁺ levels relative to wild-type controls. Likewise, inhibitors of CD38 enzyme activity also modulate NAD⁺ tissue levels and would be useful in treating various diseases where CD38 is over expressed or where cellular NAD⁺ levels are depressed or desynchronized. Compounds which inhibit CD38 and thereby raise NAD⁺ levels are useful in treating diseases or conditions indicated to benefit from NAD+ including mitochondrial-related diseases or disorders.

### SUMMARY OF THE INVENTION

There is a need for new compounds and treatments for diseases or disorders associated with CD38.

In an aspect, the present disclosure provides a compound represented by the structure of Formula I:

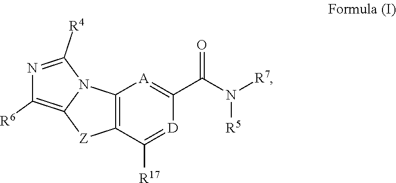

or a pharmaceutically acceptable salt or solvate thereof; wherein
Z is selected from

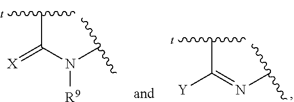

wherein t represents the point of connection between Z and the imidazole ring;
X is selected from O and S;
Y is selected from —N(R¹⁰)₂, —OR¹⁰, and —SR¹⁰;
A is selected from N and CR¹⁸;
wherein t represents the point of connection
D is selected from N and CR¹⁹;
R⁴ is selected from hydrogen, C₁₋₆ alkyl, C₁₋₆ haloalkyl, C₁₋₆ hydroxyalkyl, C₁₋₆ aminoalkyl, C₁₋₆ cyanoalkyl, C₁₋₆ alkoxyalkyl, C₁₋₆ alkyl-N(R²⁰)₂, C₃₋₅ cycloalkyl, and 3- to 6-membered heterocycle;
R⁵ is selected from hydrogen and C₁₋₆ alkyl;
R⁶ is selected from hydrogen, C₁₋₆ alkyl, halogen, C₁₋₆ haloalkyl, C₁₋₆ hydroxyalkyl, C₁₋₆ aminoalkyl, C₁₋₆ cyanoalkyl, C₁₋₆ alkoxyalkyl, C₁₋₆ alkyl-N(R²⁰)₂, C₄₋₆ cycloalkyl, and 3- to 6-membered heterocycloalkyl;
R⁷ is selected from C₃-C₁₂ carbocycle and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S(O)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle and 5- to 12-membered heterocycle, wherein the C₃-C₁₂ carbocycle and 5- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
each R¹⁰ is independently selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, and C₁-C₆ alkyl-N(R²⁰)₂;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In an aspect, the present disclosure provides a compound represented by the structure of Formula (II):

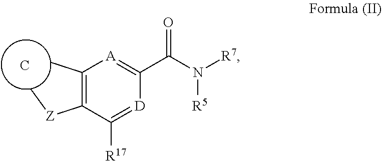

or a pharmaceutically acceptable salt or solvate thereof; wherein
Z is selected from

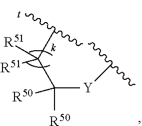

wherein t represents the point of connection between Z and \textcircled{C};
Y is selected from —O—, —NR⁹—, —S—, and —SO₂—;
each R⁵⁰ is independently selected from hydrogen, halogen, C₁-C₆ alkyl; or come together to form

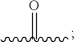 [SMILES: *=O]

each R⁵¹ is independently selected from hydrogen, halogen, and C₁-C₆ alkyl;
k is selected from 1 and 2;
A is selected from N and CR¹⁸;
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen; and C₁₋₁₀ alkyl, C₂₋₁₀ alkenyl, C₂₋₁₀ alkynyl, C₃-C₁₄ carbocycle and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, OXO, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
each R⁸* is independently selected from 4- to 12-membered heterocycle, wherein the 4- to 12-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
each R¹⁴ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, —S(O)₂(C₁₋₆ alkyl), C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In certain embodiments, the disclosure provides a pharmaceutical composition comprising a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), and a pharmaceutically acceptable excipient.

In certain embodiments, the disclosure provides a method of treating a disease or disorder, comprising administering to a subject in need thereof a compound or salt of Formula (I), Formula (IA), Formula (IB), Formula (IC), Formula (ID), Formula (II), Formula (IIA), Formula (IIB), Formula (IIC), Formula (IID), Formula (IIE), or Formula (IIF), or a pharmaceutical composition comprising a compound or salt of Formula (I), Formula (IA), Formula (IB), Formula (IC), Formula (ID), Formula (II), Formula (IIA), Formula (IIB), Formula (IIC), Formula (IID), Formula (IIE), or Formula (IIF), and a pharmaceutically acceptable excipient. In some cases, the subject would benefit from inhibition of CD38. In some cases, the disease is a neurodegenerative disease. In some cases, the disease or disorder is muscular dystrophy. In some cases, the disease or disorder is a metabolic disorder. In some cases, the disease or disorder is fibrosis. In some cases, the disease or disorder is Duchenne muscular dystrophy. In some cases, the disease or disorder is systemic sclerosis. In some cases, the disease or disorder is selected from a brain disease, vascular disease, liver disease, muscle disease, pancreas disease, adipose tissue disease, and inflammation associated disease. In some cases, the disease or disorder is selected from: neurodegenerative disease, type I diabetes, insulin resistance. Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra, Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, cystic fibrosis, pancreatitis, idiopathic pulmonary fibrosis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia—telangiectasia, rheumatoid arthritis, lupus, alcohol intolerance, hyperphosphatemia, acute lung injury, and ARDS.

In certain embodiments, the disclosure provides a method of inhibiting CD38 comprising administering a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG). In some cases, the method comprises inhibiting CD38 by administering a pharmaceutical composition comprising a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to saturated hydrocarbon groups, including straight-chain alkyl and branched-chain alkyl groups that contain from 1 to 6 carbons. The term —C_{x-y}alkylene-refers to a substituted or unsubstituted alkylene chain with from x to y carbons in the alkylene chain. For example—C₁₋₆alkylene—may be selected from methylene, ethylene, propylene, butylene, pentylene, and hexylene, any one of which is optionally substituted.

“Alkyl” as used herein refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈alkyl). In other embodiments, an alkyl comprises one to five carbon atoms (i.e., C₁-C₈alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkenyl” as used herein refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” as used herein refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” refer to unsaturated aliphatic groups analogous in length and possible substitution to the alkyls described above, but that contain at least one double or triple bond, respectively. The term —C_{x-y}alkenylene-refers to a substituted or unsubstituted alkenylene chain with from x to y carbons in the alkenylene chain. For example. —C₂₋₆alkenylene—may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkynylene chain. For example. —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. An alkylene chain may be optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. An alkenylene chain may be optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. An alkynylene chain may be optionally substituted by one or more substituents such as those substituents described herein.

“Halo” or “halogen” as used herein refers to halogen substituents such as bromo, chloro, fluoro and iodo substituents.

“Haloalkyl” as used herein refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, and any other suitable combinations of alkanes (or substituted alkanes) and halogens. When an alkyl group is substituted with more than one halogen radicals, each halogen may be independently selected, for example 1-chloro,2-bromoethane.

“Aminoalkyl” refers to an alkyl radical, as defined above, that is substituted by one or more amine radicals, for example, propan-2-amine, butane-1,2-diamine, pentane-1,2,4-triamine and the like.

“Hydroxyalkyl” refers to an alkyl radical, as defined above, that is substituted by one or more hydroxy radicals, for example, propan-1-ol, butane-1,4-diol, pentane-1,2,4-triol, and the like.

“Alkoxyalkyl” refers to an alkyl radical, as defined above, that is substituted by one or more alkoxy radicals, for example, methoxymethane, 1,3-dimethoxybutane, 1-methoxypropane, 2-ethoxypentane, and the like.

“Cyanoalkyl” as used herein refers to an alkyl radical, as defined above, that is substituted by one or more cyano radicals, for example, acetonitrile, 2-ethyl-3-methylsuccinonitrile, butyronitrile, and the like.

The term “carbocycle” as used herein refers to a saturated, unsaturated or aromatic ring in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Bicyclic carbocycles may be fused, bridged or spiro-ring systems. A carbocycle may be optionally substituted by one or more substituents such as those substituents described herein.

The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “cycloalkyl” as used herein refers to a saturated carbocycle. Exemplary cycloalkyl rings include cyclopropyl, cyclohexyl, and norbornane. Carbocycles may be optionally substituted by one or more substituents such as those substituents described herein.

The term “C_{x-y} carbocycle” is meant to include groups that contain from x to y carbons in the cycle. For example, the term “C₃₋₆ carbocycle” refers to a saturated, unsaturated, or aromatic ring comprising from 3 to 6 carbons. For example-C₃₋₆ carbocycle- may be selected from cyclopropyl, cyclobutyl, cyclopentyl, cyclohexyl, and phenyl, any one of which is optionally substituted.

“Aryl” as used herein refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene.

The term “heterocycle” as used herein refers to a saturated, unsaturated or aromatic ring comprising one or more heteroatoms. Exemplary heteroatoms include N, O, Si, P, B, and S atoms. The heterocycle may be attached to the rest of the molecule through any atom of the heterocycle, valence permitting, such as a carbon or nitrogen atom of the heterocycle. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Bicyclic heterocycles may be fused, bridged, or spiro-ring systems. A spiro-ring system may be referred as a “spiro heterocycle” or “spiroheterocycle” or “spiro-ring heterocycle”. In some cases, spiro heterocycle, spiro-ring heterocycles or spiroheterocycles have at least two molecular rings with only one common atom. The spiro heterocycle, spiro-ring heterocycle or spiroheterocycle comprises one or more heteroatoms.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine.

An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. Heterocycles may be optionally substituted by one or more substituents such as those substituents described herein.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b} —N(R^{a}) S(O)R^{a} (where t is 1 or 2), —R^{b}—S(O); R^{a} (where t is 1 or 2), —R^{b}—S(O); OR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (—O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—)OR^{a}, —R^{b}—OC(O)—N(R^{a})₂, —R^{b}—N(R^{a})₂, —R^{b}—C(O)R^{a}, —R^{b} —C(O)OR^{a}, —R^{b}—C(O)N(R^{a})₂, —R^{b}—O—R^{c}—C(O)N(R^{a})₂, —R^{b}—N(R^{a})C(O))OR^{a}, —R^{b}—N(R^{a})C(O)R^{a}, —R^{b}—N(R^{a}) S(O); R^{a} (where t is 1 or 2), —R^{b}—S(O); R^{a} (where t is 1 or 2), —R^{b}—S(O); OR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl.

heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N-NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b} —C(O)OR^{a}, —R^{b}—C(O)N(R^{a})₂, —R^{b}—O—R^{c}—C(O)N(R^{a})₂, —R^{b}—N(R^{a})C(O))OR^{a}, —R^{b}—N(R^{a})C(O)R^{a}, —R^{b}—N(R^{a}) S(O); R^{a} (where t is 1 or 2), —R^{b}—S(O)R^{a} (where t is 1 or 2), —R^{b}—S(O); OR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each Re is a straight or branched alkylene, alkenylene or alkynylene chain. It will be understood by those skilled in the art that substituents can themselves be substituted, if appropriate.

As used herein, the term “optional” or “optionally” means that the subsequently described event of circumstances may or may not occur, and that the description includes instances where the event or circumstance occurs and instances in which it does not. For example. “optionally substituted aryl” means that the aryl group may or may not be substituted and that the description includes both substituted aryl groups and aryl groups having no substitution.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate: (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “subject.” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the invention or a prodrug of a compound of the invention to the individual in need.

The term “effective amount” or “therapeutically effective amount” refers to that amount of a compound or salt described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein. “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “selective inhibition” or “selectively inhibit” as referred to a biologically active agent refers to the agent's ability to preferentially reduce the target signaling activity as compared to off-target signaling activity, via direct or interact interaction with the target.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The section headings used herein are for organizational purposes only and are not to be construed as limiting the subject matter described.

### Compounds

In an aspect, the present disclosure provides a compound represented by the structure of Formula (I):

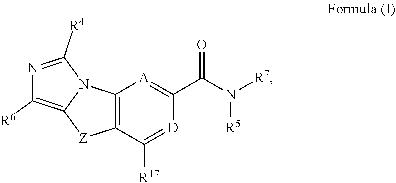

or a pharmaceutically acceptable salt or solvate thereof; wherein
Z is selected from

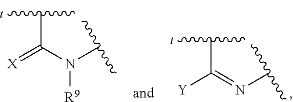

wherein t represents the point of connection between Z and the imidazole ring;
X is selected from O and S;
Y is selected from —N(R¹⁰)₂, —OR¹⁰, and —SR¹⁰;
A is selected from N and CR¹⁸;
D is selected from N and CR¹⁹;
R⁴ is selected from hydrogen, C₁₋₆ alkyl, C₁₋₆ haloalkyl, C₁₋₆ hydroxyalkyl, C₁₋₆ aminoalkyl, C₁₋₆ cyanoalkyl, C₁₋₆ alkoxyalkyl, C₁₋₆ alkyl-N(R²⁰)₂, C₃₋₅ cycloalkyl, and 3- to 6-membered heterocycle;
R⁵ is selected from hydrogen and C₁₋₆ alkyl;
R⁶ is selected from hydrogen, C₁₋₆ alkyl, halogen, C₁₋₆ haloalkyl, C₁₋₆ hydroxyalkyl, C₁₋₆ aminoalkyl, C₁₋₆ cyanoalkyl, C₁₋₆ alkoxyalkyl, C₁₋₆ alkyl-N(R²⁰)₂, C₄₋₆ cycloalkyl, and 3- to 6-membered heterocycloalkyl;
R⁷ is selected from C₃-C₁₂ carbocycle and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle and 5- to 12-membered heterocycle, wherein the C₃-C₁₂ carbocycle and 5- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
each R¹⁰ is independently selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, and C₁-C₆ alkyl-N(R²⁰)₂;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰—C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (I), Formula (II), Formula (IIA), Formula (IIB), or Formula (IIC), A is N. In some cases, A is CR¹⁸. In some cases, R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹⁸ is selected from hydrogen, halogen, —OH, —CN, —NO₂, —NH₂, —O—C₁₋₁₀ alkyl, C₁-C₆ alkyl, and C₁-C₆ haloalkyl. In some cases, R¹⁸ is selected from hydrogen, halogen, —OH, —CN, —O—C₁₋₁₀ alkyl, C₁-C₆ alkyl, and C₁-C₆ haloalkyl. In some cases, R¹⁸ is selected from hydrogen, halogen, —OH, —CN, C₁-C₆ alkyl, and C₁-C₆ haloalkyl. In some cases, R¹⁸ is selected from hydrogen, halogen, —OH, —CN, C₁-C₄ alkyl, and C₁-C₄ haloalkyl. In some cases, R¹⁸ is selected from hydrogen, halogen, —OH, —CN, methyl, ethyl, propyl, isopropyl, trifluoromethyl, difluoromethyl, and trifluoroethyl. In some cases, R¹⁸ is selected from hydrogen, halogen, —OH, —CN, methyl, and trifluoromethyl. In some cases, R¹⁸ is selected from hydrogen, fluorine, —OH, —CN, methyl, and trifluoromethyl. In some cases, R¹⁸ is selected from hydrogen, fluorine, —OH, and methyl. In some cases, R¹⁸ is selected from hydrogen, fluorine, and methyl. In some cases, R¹⁸ is methyl. In some cases, R¹⁸ is fluorine.

In some embodiments, for a compound or salt of Formula (I), Formula (II), Formula (IIA), Formula (IIB), or Formula (IIC), D is N. In some cases, D is CR¹⁹. In some cases, R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹⁹ is selected from hydrogen, halogen, —OH, —CN, —NO₂, —NH₂, —O—C₁₋₁₀ alkyl, C₁-C₆ alkyl, and C₁-C₆ haloalkyl. In some cases, R¹⁹ is selected from hydrogen, halogen, —OH, —CN, —O—C₁₋₁₀ alkyl, C₁-C₆ alkyl, and C₁-C₆ haloalkyl. In some cases, R¹⁹ is selected from hydrogen, halogen, —OH, —CN, C₁-C₆ alkyl, and C₁-C₆ haloalkyl. In some cases, R¹⁹ is selected from hydrogen, halogen, —OH, —CN, C₁-C₄ alkyl, and C₁-C₄ haloalkyl. In some cases, R¹⁹ is selected from hydrogen, halogen, —OH, —CN, methyl, ethyl, propyl, isopropyl, trifluoromethyl, difluoromethyl, and trifluoroethyl. In some cases, R¹⁹ is selected from hydrogen, halogen, —OH, —CN, methyl, and trifluoromethyl. In some cases, R¹⁹ is selected from hydrogen, fluorine, —OH, —CN, methyl, and trifluoromethyl. In some cases, R¹⁹ is selected from hydrogen, fluorine, —OH, and methyl. In some cases, R¹⁹ is selected from hydrogen, fluorine, and methyl. In some cases, R¹⁹ is methyl. In some cases, R¹⁹ is fluorine.

In some embodiments, Formula (I) is represented by Formula (IA):

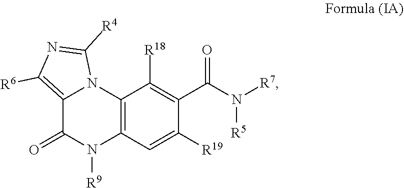

or a pharmaceutically acceptable salt or solvate thereof.

In some embodiments, Formula (I) is represented by Formula (IB):

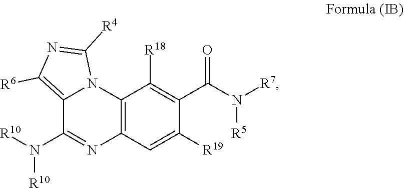

or a pharmaceutically acceptable salt or solvate thereof.

In some embodiments, for a compound or salt of Formula (I), Formula (IA), or Formula (IB), R⁴ is selected from hydrogen, C₁₋₆ alkyl, C₁₋₆ haloalkyl, C₁₋₆ hydroxyalkyl, C₁₋₆ aminoalkyl, C₁₋₆ cyanoalkyl, C₁₋₆ alkoxyalkyl, and C₁₋₆ alkyl-N(R²⁰) 2. In some cases, R⁴ is selected from hydrogen, C₁₋₆ alkyl, and C₁₋₆ haloalkyl. In some cases, R⁴ is hydrogen.

In some embodiments, for a compound or salt of Formula (I), Formula (IA), or Formula (IB), R⁶ is selected from hydrogen, C₁₋₆ alkyl, halogen, C₁₋₆ haloalkyl, C₁₋₆ hydroxyalkyl, C₁₋₆ aminoalkyl, C₁₋₆ cyanoalkyl, C₁₋₆ alkoxyalkyl, and C₁₋₆ alkyl-N(R²⁰) 2. In some cases, R⁶ is hydrogen.

In some embodiments, for a compound or salt of Formula (I), Formula (IA), or Formula (IB), R⁵ is selected from hydrogen and methyl. In some cases, R⁵ is hydrogen.

In some embodiments, for a compound or salt of Formula (I), Formula (IA), Formula (IC), or Formula (II), R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle. In some cases, R⁹ is selected hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰) 2. In some cases, R⁹ is selected from hydrogen and C₁₋₆ alkyl. In some cases, R⁹ is selected from hydrogen and methyl. In some cases, R⁹ is hydrogen. In some cases, R⁹ is selected from C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl. In some cases, R⁹ is hydrogen. In some cases, R⁹ is selected from C₃₋₆ carbocycle and 4- to 6-membered heterocycle, wherein the C₃₋₆ carbocycle and 4- to 6-membered heterocycle are each optionally substituted with one or more substituents independently selected from halogen, —OH, C₁₋₁₀ alkyl, —C₁₋₁₀ haloalkyl, and —O—C₁₋₁₀ alkyl.

In some embodiments, for a compound or salt of Formula (I), Formula (IB), or Formula (ID), each R¹⁰ is selected from hydrogen, C₁-C₆ alkyl, and C₁-C₆ haloalkyl. In some cases, each R¹⁰ is selected from hydrogen and C₁₋₆ alkyl. In some cases, at least one occurrence of R¹⁰ is hydrogen. In some cases, each R¹⁰ is independently selected from hydrogen, C₁₋₆ alkyl, and C₁-C₆ alkoxyalkyl. In some cases, each R¹⁰ is independently selected from C₁-C₆ alkoxyalkyl. In some cases, each R¹⁰ is independently selected from C₁-C₂ alkoxyalkyl. In some cases, each R¹⁰ is independently selected from

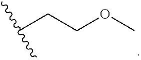 [SMILES: *CCOC]

In some embodiments, Formula (I) is represented by Formula (IC):

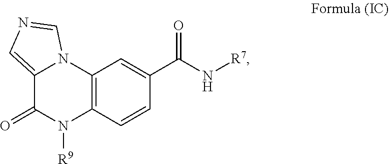

or a pharmaceutically acceptable salt or solvate thereof.

In some embodiments, Formula (I) is represented by Formula (ID):

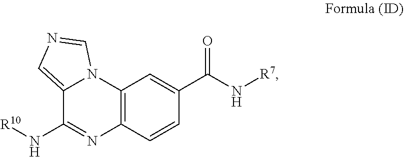

or a pharmaceutically acceptable salt or solvate thereof.

In some embodiments, for a compound or salt of Formula (I), Formula (IA), or Formula (IC), R⁹ is selected from hydrogen and methyl.

In some embodiments, for a compound or salt of Formula (I), Formula (IB), or Formula (ID), R¹⁰ is selected from hydrogen and methyl. In some cases, R¹⁰ is hydrogen. In some cases, R¹⁰ is methyl.

In an aspect, the present disclosure provides a compound represented by the structure of Formula (II):

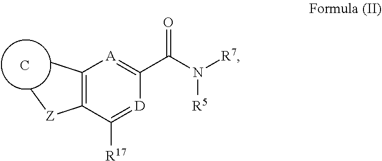

or a pharmaceutically acceptable salt or solvate thereof; wherein
Z is selected from

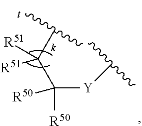

wherein t represents the point of connection between Z and \textcircled{C};
Y is selected from —O—, —NR⁹—, —S—, and —SO₂—;
each R⁵⁰ is independently selected from hydrogen, halogen, C₁-C₆ alkyl; or come together to form

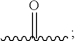 [SMILES: *=O]

each R⁵¹ is independently selected from hydrogen, halogen, and C₁-C₆ alkyl;
k is selected from 1 and 2;
A is selected from N and CR¹⁸;
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen, C₁₋₁₀ alkyl, C₂₋₁₀ alkenyl, C₂₋₁₀ alkynyl, C₃-C₁₄ carbocycle and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, OXO, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
each R⁸* is independently selected from 4- to 12-membered heterocycle, wherein the 4- to 12-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;

R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, —S(O)₂(C₁₋₆ alkyl), C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (II) Z is

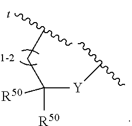

In some cases, K is 1. In some cases, K is 2. In some cases, Z is

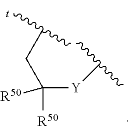

In some embodiments, for a compound or salt of Formula (II), each R⁵¹ is independently selected from hydrogen, halogen, and C₁-C₆ alkyl. In some cases, each R⁵¹ is independently selected from hydrogen, halogen, methyl, ethyl, and isopropyl. In some cases, each R⁵¹ is independently selected from hydrogen, F, Cl, methyl, ethyl, and isopropyl. In some cases, each R⁵¹ is independently selected from hydrogen, F, and methyl. In some cases, each R⁵¹ is independently selected from hydrogen and methyl. In some cases, each R⁵¹ is independently selected from hydrogen and F. In some cases, each R⁵¹ is hydrogen.

In some embodiments, for a compound or salt of Formula (II): Z is selected from

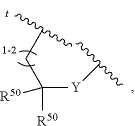

wherein t represents the point of connection between Z and \textcircled{C};
Y is selected from —O—, —NR⁹—, —S—, and —SO₂—;
each R⁵⁰ is independently selected from hydrogen, halogen, C₁-C₆ alkyl; or come together to form

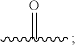 [SMILES: *=O]

A is selected from N and CR¹⁸;
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen, C₁₋₁₀ alkyl, C₂₋₁₀ alkenyl, C₂₋₁₀ alkynyl, C₃-C₁₄ carbocycle and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 5- to 12-membered heterocycle, wherein the C₃-C₁₂ carbocycle and 5- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (I), Formula (IA), Formula (IB), Formula (II), Formula (IIA), Formula (IIB), Formula (IIC), Formula (IID), Formula (IIE), Formula (IIF), or Formula (IIG), R⁵ is selected from hydrogen and C₁-C₆ alkyl. In some cases, R⁵ is selected from hydrogen and C₁-C₄ alkyl. In some cases, R⁵ is selected from hydrogen, methyl, ethyl, propyl, and isopropyl. In some cases, R⁵ is selected from hydrogen, methyl, and ethyl. In some cases, R⁵ is selected from hydrogen and methyl. In some cases, R⁵ is hydrogen. In some cases, R⁵ is methyl.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₆-C₈ carbocycle and 5- to 6-membered heterocycle, each of which are optionally substituted with one or more R⁸. In some cases, R⁷ is selected from C₆-C₈ carbocycle, which is optionally substituted with one or more R⁸. In some cases, R⁷ is selected from C₆ carbocycle, which is optionally substituted with one or more R⁸. In some cases, R⁷ is selected from C₆ cycloalkyl, which is optionally substituted with one or more R⁸. In some cases, R⁷ is selected from C₇ carbocycle, which is optionally substituted with one or more R⁸. In some cases, R⁷ is selected from C₈ carbocycle, which is optionally substituted with one or more R⁸. In some cases, R⁷ is

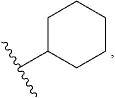 [SMILES: *C1CCCCC1]

substituted at the para position. In some cases, each R⁸ is independently selected from halogen, —OR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle and 5- to 12-membered heterocycle. In some cases, each R⁸ is independently selected from halogen, —OR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —N(R²⁰)C(O)R²⁰, —N(R²⁰)C(O)N(R²⁰)₂, —N(R²⁰)C(O)OR²⁰, —C(O)OR²⁰, —OC(O)R²⁰, —OC(O)N(R²⁰)₂, ═O, —CN, C₁₋₆ alkyl-N(R²⁰)₂, C₁₋₆ haloalkyl, C₁₋₆ alkyl, C₃-C₁₂ carbocycle and 5- to 12-membered heterocycle. In some cases, each R⁸ is independently selected from fluoro, chloro, —OR²⁰, —S(O)₂(R²⁰), —S)₂N(R²⁰)₂, —NR²⁰S(O)₂R²⁰, —C(O)N(R²⁰)₂, —N(R²⁰)C(O)R²⁰, —C(O)OR²⁰, —OC(O)R²⁰—OC(O)N(R²⁰) 2, =0, —CN, C₁₋₆ alkyl, C₃-C₁₂ carbocycle and 5- to 12-membered heterocycle. In some cases, each R⁸ is independently selected from fluoro, —OR²⁰, —S(O)₂(R²⁰), —S)₂N(R²⁰)₂, ═O, and C₁₋₆ alkyl. In some cases, each R⁸ is independently selected from fluoro, —OR²⁰, —S(O)₂(R²⁰), —S)₂N(R²⁰)₂, ═O, and C₁₋₄ alkyl. In some cases, each R⁸ is independently selected from fluoro, —OR²⁰, —S(O)₂(R²⁰), —S)₂N(R²⁰)₂, methyl, ethyl, propyl, and isopropyl. In some cases, each R⁸ is independently selected from fluoro, —OR²⁰, —S(O)₂(R²⁰), methyl, and ethyl. In some cases, R⁸ is —S(O)₂(R²⁰). In some cases, each R⁸ is —OR²⁰. In some cases, each R⁸ is fluoro. In some cases, each R⁸ is methyl. In some cases, each R⁸ is —S(O)₂(R²⁰). In some cases, R⁷ is

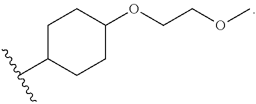 [SMILES: *C1CCC(OCCOC)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰—N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, OXO, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, —N(R²⁰)C(O)R²⁰, —N(R²⁰)C(O)N(R²⁰)₂, —N(R²⁰)C(O)OR²⁰, —C(O)OR²⁰, —OC(O)N(R²⁰)₂, —CN, C₁₋₆ haloalkyl, C₁₋₆ hydroxyalkyl, C₁₋₆ alkyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, C₁₋₆ haloalkyl, C₁₋₆ hydroxyalkyl, C₁₋₆ alkyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, C₁₋₆ haloalkyl, C₁₋₆ hydroxyalkyl, C₁₋₆ alkyl, C₃-C₈ carbocycle, and 4- to 8-membered heterocycle, wherein the C₁₋₆ alkyl is optionally substituted with one or more R⁸*, and wherein the C₃-C₈ carbocycle and 4- to 8-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, C₁₋₆ haloalkyl, C₁₋₆ hydroxyalkyl, C₁₋₆ alkyl, C₃-C₈ cycloalkyl, and 4- to 8-membered heterocycle, wherein the C₁₋₆ alkyl is optionally substituted with one or more R⁸*, and wherein the C₃-C₈ cycloalkyl and 4- to 8-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, R⁸ is independently selected from halogen, —N(R²⁰)₂, —OR²⁰, —NR²⁰S(O)₂R²⁰, —C(O)N(R²⁰)₂, C₁₋₆ haloalkyl, C₁₋₆ hydroxyalkyl, C₁₋₆ alkyl, C₃-C₈ cycloalkyl, and 4- to 8-membered heteroaryl, wherein the C₁₋₆ alkyl is optionally substituted with one or more R⁸*, and wherein the C₃-C₈ cycloalkyl and 4- to 8-membered heteroaryl are each optionally substituted with one or more substituents selected from halogen, —OH, —CN, —NO₂, —NH₂, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸* is independently selected from 4- to 12-membered heterocycle, wherein the 4- to 12-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, each R⁸* is independently selected from 4- to 10-membered heterocycle, wherein the 4- to 10-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl. In some cases, each R⁸* is independently selected from 4- to 10-membered heterocycle, wherein the 4- to 10-membered heterocycle is optionally substituted with one or more substituents selected from F, Cl, —OH, —CN, —NH₂, C₁₋₆ alkyl, —C₁₋₆ haloalkyl, —O-C₁₋₆ alkyl, C₁-C₆ hydroxyalkyl, C₁-C₆ aminoalkyl, C₁-C₆ cyanoalkyl, and C₁-C₆ alkoxyalkyl. In some cases, each R⁸* is independently selected from 4- to 10-membered heterocycle, wherein the 4- to 10-membered heterocycle is optionally substituted with one or more substituents selected from F, Cl, —OH, —CN, methyl, ethyl, isopropyl, —CH₂F, —CHF₂, —CHF₃, —CH₂CF₃, —CF₂CF₃, —OCH₃, —OCH₂CH₃, and —OCH(CH₃)₂. In some cases, each R⁸* is independently selected from 4- to 8-membered heterocycle, wherein the 4- to 8-membered heterocycle is optionally substituted with one or more substituents selected from F, Cl, —OH, —CN, —NH₂, C₁₋₆ alkyl, —C₁₋₆ haloalkyl, —O—C₁₋₆ alkyl, C₁-C₆ hydroxyalkyl, C₁-C₆ aminoalkyl, C₁-C₆ cyanoalkyl, and C₁-C₆ alkoxyalkyl. In some cases, each R⁸* is independently selected from 4- to 8-membered heterocycle, wherein the 4- to 8-membered heterocycle is optionally substituted with one or more substituents selected from F, Cl, —OH, —CN, methyl, ethyl, isopropyl, —CH₂F, —CHF₂, —CHF₃, —CH₂CF₃, —CF₂CF₃, —OCH₃, —OCH₂CH₃, and —OCH(CH₃)₂.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₆-C₈ carbocycle and 5- to 6-membered heterocycle, each of which are optionally substituted with one or more R⁸. In some cases, R⁷ is selected from C₆-C₈ carbocycle, which is optionally substituted with one or more R⁸. In some cases, R⁷ is selected from C₆ carbocycle, which is optionally substituted with one or more R⁸. In some cases, R⁷ is selected from C₆ cycloalkyl, which is optionally substituted with one or more R⁸. In some cases, R⁷ is selected from C₇ carbocycle, which is optionally substituted with one or more R⁸. In some cases, R⁷ is selected from C₈ carbocycle, which is optionally substituted with one or more R⁸. In some cases R⁷ is selected from C₄-C₆ carbocycle, which is optionally substituted with one or more R⁸. In some cases R⁷ is selected from C₅ carbocycle, which is optionally substituted with one or more R⁸. In some cases R⁷ is selected from C₅ cycloalkyl, which is optionally substituted with one or more R⁸. In some cases R⁷ is selected from C₄ carbocycle, which is optionally substituted with one or more R. In some cases R⁷ is selected from C₄ cycloalkyl, which is optionally substituted with one or more R⁸. In some cases, R⁷ is selected from

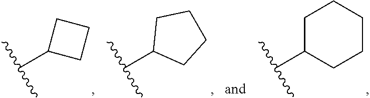

which is optionally substituted with one or more R⁸. In some cases, R⁷ is selected from and

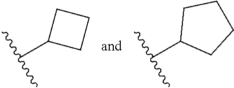 [SMILES: *C1CCC1 ; *C1CCCC1]

and, which is optionally substituted with one or more R⁸. In some cases, R⁷ is selected from

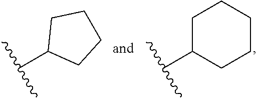 [SMILES: *C1CCCC1 ; *C1CCCCC1]

which is optionally substituted with one or more R⁸. In some cases, R⁷ is selected from

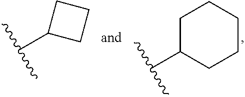 [SMILES: *C1CCC1 ; *C1CCCCC1]

which is optionally substituted with one or more R⁸. In some cases, R⁷ is selected from

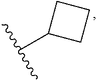 [SMILES: *C1CCC1]

which is optionally substituted with one or more R⁸. In some cases, R⁷ is selected from

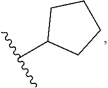 [SMILES: *C1CCCC1]

which is optionally substituted with one or more R⁸. In some cases, R⁷ is selected from

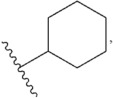 [SMILES: *C1CCCCC1]

which is optionally substituted with one or more R⁸. In some cases, the C₄ carbocycle is substituted at the para position. In some cases, the C₅ carbocycle is substituted at the 3 position. In some cases, the C₅ carbocycle is substituted at the 2 position. In some cases, the C₆ carbocycle is substituted at the para position.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —S(O)₂(R²⁰), —S)₂N(R²⁰)₂, —S(O)N(R²⁰)₂, —S(O)R²⁰(═NR²⁰), —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —S(O)₂(R²⁰), —S)₂N(R²⁰)₂, —S(O)N(R²⁰)₂, —S(O)R²⁰(═NR²⁰), —C(O)N(R²⁰)₂, —N(R²⁰)C(O)R²⁰, —N(R²⁰)C(O)N(R²⁰)₂, —N(R²⁰)C(O)OR²⁰, —C(O)OR²⁰, —OC(O)R²⁰, —OC(O)N(R²⁰)₂, C₃-C₁₂ carbocycle and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —NR²⁰S(O)₂R²⁰, —N(R²⁰)C(O)R²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —NR²⁰S(O)₂R²⁰, —N(R²⁰)C(O)R²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently selected from halogen, C₁₋₆ alkyl, C₁₋₆ haloalkyl, C₁₋₆ hydroxyalkyl, —OR²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently selected from halogen, C₁₋₆ alkyl, C₁₋₆ haloalkyl, C₁₋₆ hydroxyalkyl, —OR²⁰, —C(O)N(R²⁰)₂, and 4- to 6-membered heterocycle, wherein the 4- to 6-membered heterocycle is optionally substituted with one or more substituents selected from F, Cl, —OH, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently, selected from F,

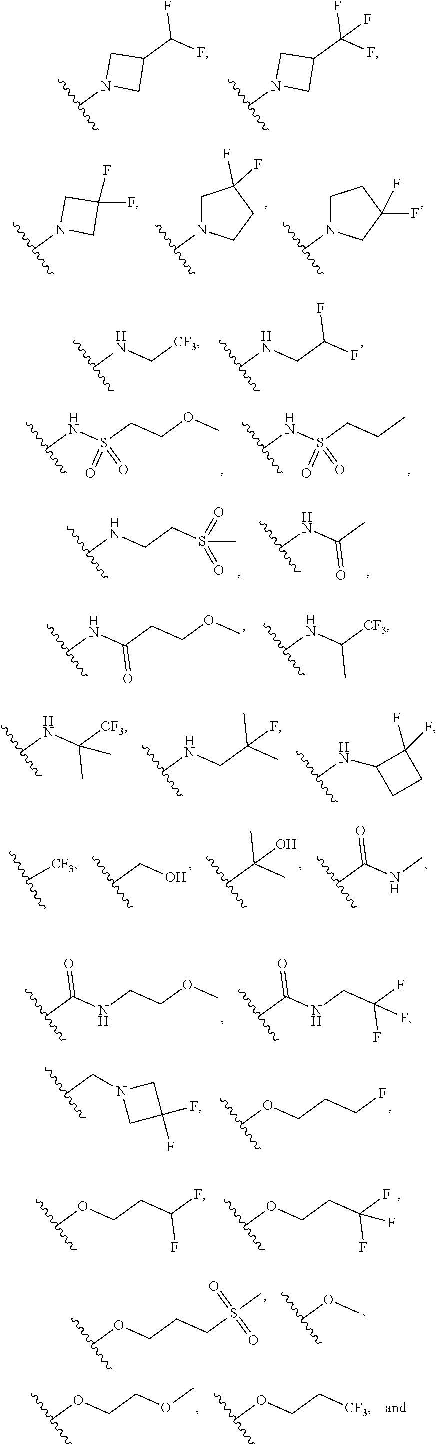

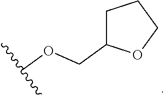 [SMILES: *OCC1CCCO1]

In some cases. R⁷ is

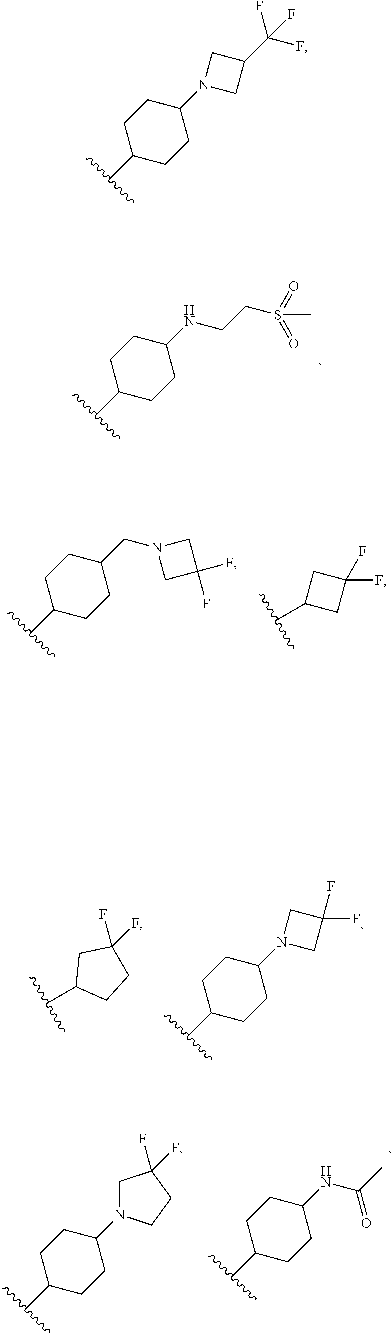

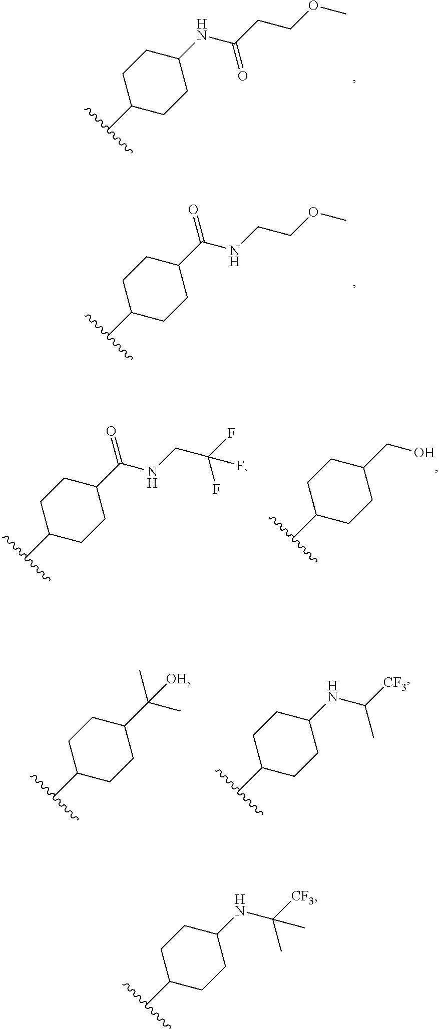

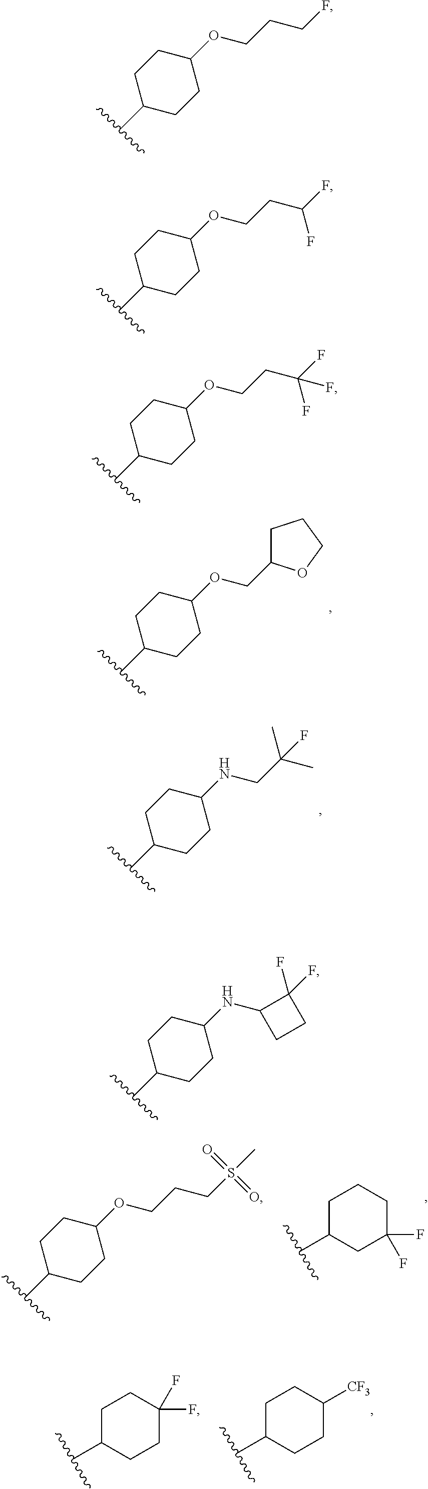

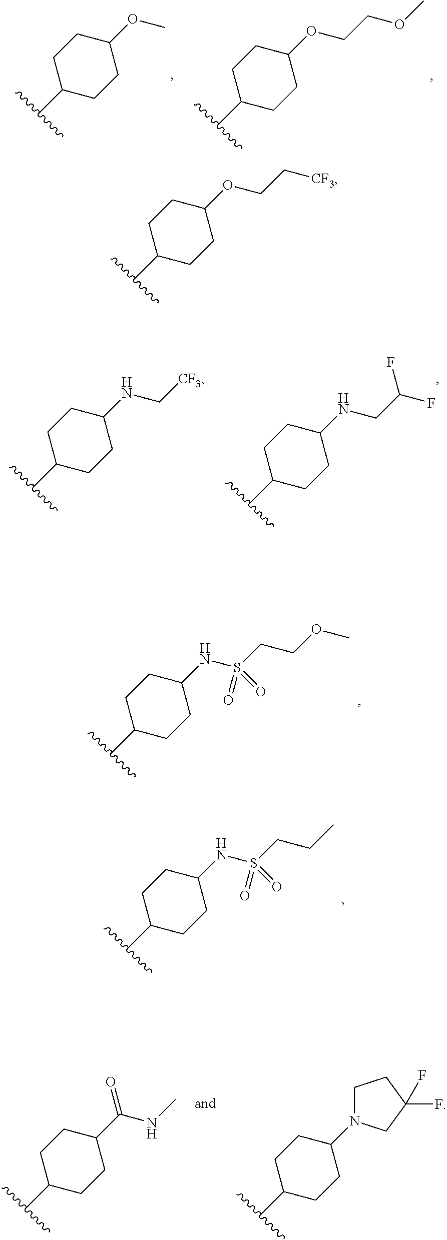

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is

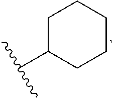 [SMILES: *C1CCCCC1]

which is optionally substituted with one or more R⁸. In some cases R⁷ is

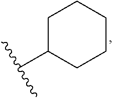 [SMILES: *C1CCCCC1]

which is optionally substituted with one or two R⁸. In some cases, each R⁸ is independently selected from halogen, C₁₋₆ haloalkyl, —OR²⁰, —N(R²⁰)₂, —NR²⁰S(O)₂R²⁰, C₁₋₆ alkyl-N(R²⁰)₂, —C(O)N(R²⁰)₂, and 5- to 6-membered heterocycle, wherein the 5- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl. In some cases, each R⁸ is independently selected from —N(R²⁰)₂, and C₁₋₆ alkyl-N(R²⁰) 2. In some cases, each R⁸ is independently selected from —N(R²⁰) 2. In some cases, each R⁸ is independently selected from fluorine, —CH₃, —CF₃, —OH, —OCH₃,

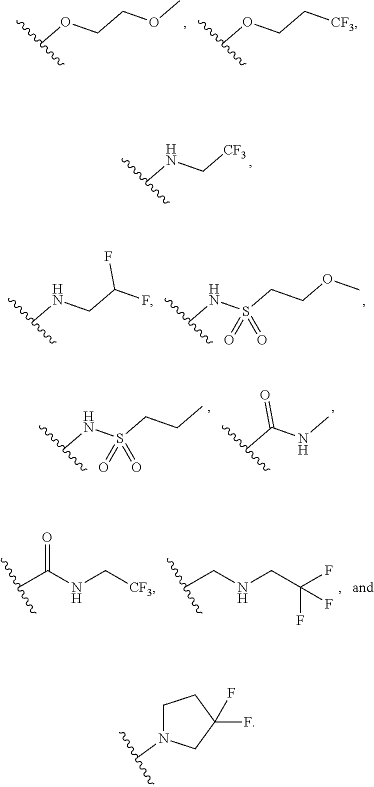

In some cases, R⁷ is selected from

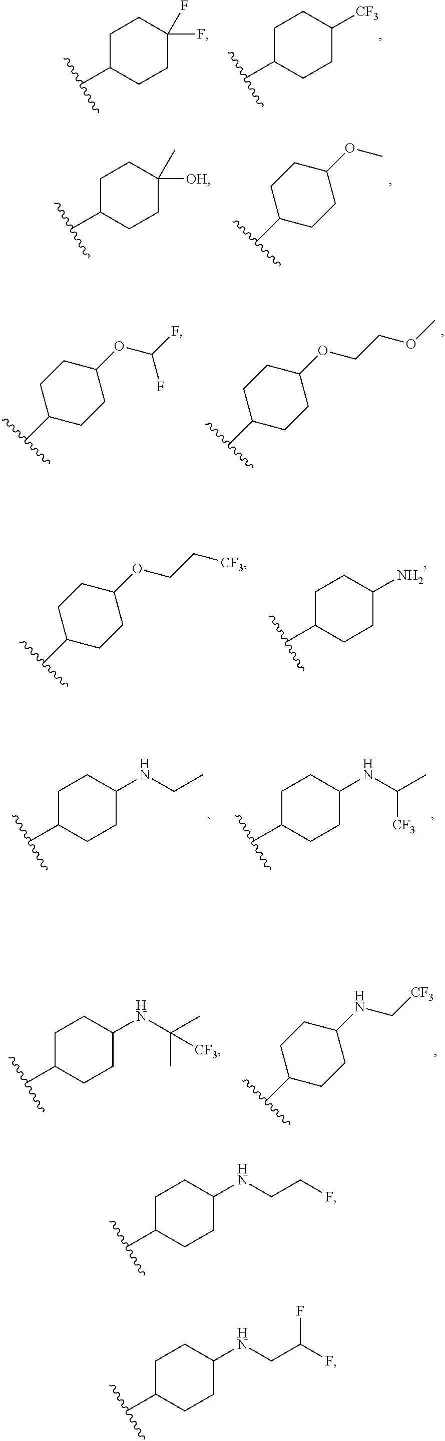

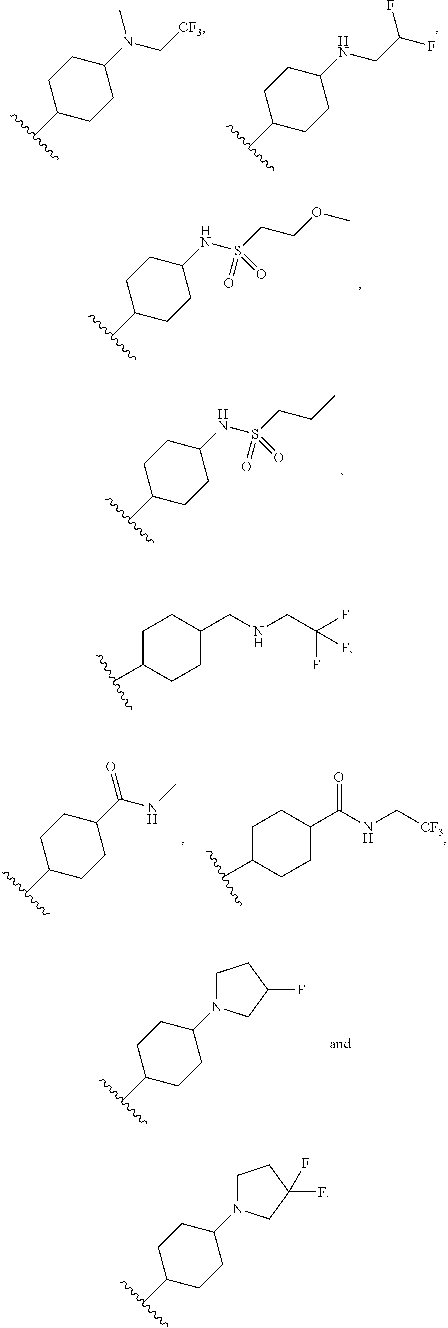

In some cases, each R⁸ is independently selected from halogen and C₁₋₆ haloalkyl. In some cases, each R⁸ is independently selected from fluorine and —CF₃. In some cases, In some cases, R⁷ is selected from

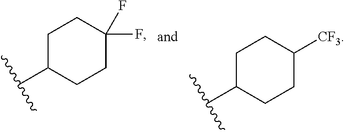 [SMILES: *C1CCC(F)(F)CC1 ; *C1CCC(C(F)(F)F)CC1]

In some cases, each R⁸ is independently selected from —OR²⁰. In some cases, R²⁰ is independently selected at each occurrence from C₁₋₆ alkyl, wherein the C₁₋₆ alkyl is optionally substituted with one or more substituents independently selected from halogen, and —O—C₁₋₁₀ alkyl. In some cases, each R⁸ is independently selected from —OCH₃,

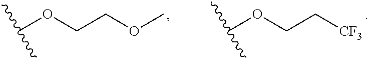

In some cases, In some cases, R⁷ is selected from

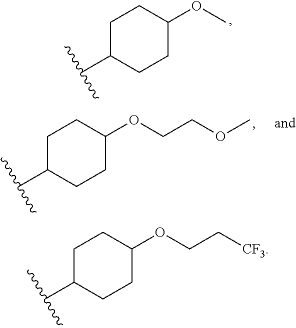

In some cases, each R⁸ is independently selected from —N(R²⁰)₂. In some cases, R²⁰ is independently selected at each occurrence from hydrogen and C₁₋₆ alkyl, wherein the C₁₋₆ alkyl is optionally substituted with one or more substituents independently selected from halogen. In some cases, each R²⁰ is different. In some cases, each R⁸ is independently selected from

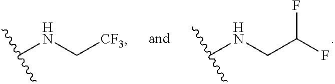 [SMILES: *NCC(F)(F)F ; *NCC(F)F]

In some cases, R⁷ is selected from

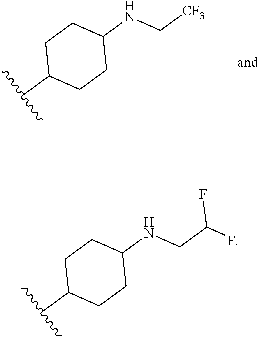 [SMILES: *C1CCC(NCC(F)(F)F)CC1 ; *C1CCC(NCC(F)F)CC1]

In some cases, each R⁸ is independently selected from —NR²⁰S(O)₂R²⁰. In some cases, each R⁸ is independently selected from —NHS(O)₂R²⁰. In some cases, R²⁰ is independently selected at each occurrence from C₁₋₆ alkyl, wherein the C₁₋₆ alkyl is optionally substituted with one or more substituents independently selected from —O—C₁₋₁₀ alkyl. In some cases, each R⁸ is independently selected from

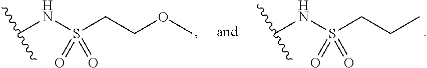

In some cases, R⁷ is selected from

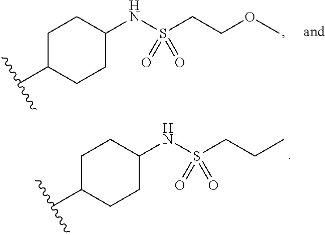 [SMILES: *C1CCC(NS(=O)(=O)CCOC)CC1 ; *C1CCC(NS(=O)(=O)CCC)CC1]

In some cases, each R⁸ is independently selected from —C(O)N(R²⁰)₂. In some cases, R²⁰ is independently selected at each occurrence from hydrogen and C₁₋₆ alkyl. In some cases, each R⁸ is independently selected from

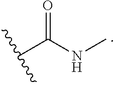 [SMILES: *C(=O)NC]

In some cases, R⁷ is

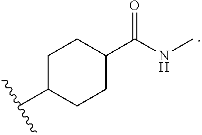 [SMILES: *C1CCC(C(=O)NC)CC1]

In some cases, each R⁸ is independently selected from 5- to 6-membered heterocycle, wherein the 5- to 6-membered heterocycle is optionally substituted with one or more substituents selected from halogen, C₁₋₁₀ alkyl, —C₁₋₁₀ haloalkyl, and —O—C₁₋₁₀ alkyl. In some cases, each R⁸ is independently selected from 5- to 6-membered heterocycle, wherein the 5- to 6-membered heterocycle is optionally substituted with one or more substituents selected from halogen. In some cases, each R⁸ is independently selected from

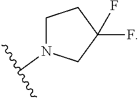 [SMILES: *N1CCC(F)(F)C1]

In some cases, R⁷ is

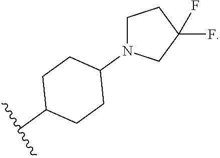 [SMILES: *C1CCC(N2CCC(F)(F)C2)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from

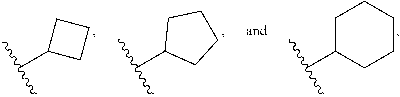 [SMILES: *C1CCC1 ; *C1CCCC1 ; *C1CCCCC1]

which is optionally substituted with one or more R⁸. In some cases, R⁷ is selected from

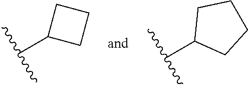 [SMILES: *C1CCC1 ; *C1CCCC1]

and, which is optionally substituted with one or more R⁸. In some cases, R⁷ is selected from

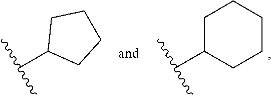 [SMILES: *C1CCCC1 ; *C1CCCCC1]

which is optionally substituted with one or more R⁸. In some cases, R⁷ is selected from

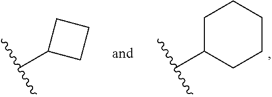 [SMILES: *C1CCC1 ; *C1CCCCC1]

which is optionally substituted with one or more R⁸. In some cases, R⁷ is selected from

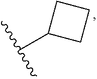 [SMILES: *C1CCC1]

which is optionally substituted with one or more R⁸. In some cases, R⁷ is selected from

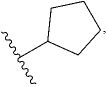 [SMILES: *C1CCCC1]

which is optionally substituted with one or more R⁸. In some cases R⁷ is

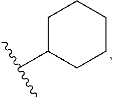 [SMILES: *C1CCCCC1]

which is optionally substituted with one or two R⁸. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —NR²⁰S(O)₂R²⁰, —N(R²⁰)C(O)R²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —NR²⁰S(O)₂R²⁰, —N(R²⁰)C(O)R²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently selected from halogen, C₁₋₆ alkyl, C₁₋₆ haloalkyl, C₁₋₆ hydroxyalkyl, —OR²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently selected from halogen, C₁₋₆ alkyl, C₁₋₆ haloalkyl, C₁₋₆ hydroxyalkyl, —OR²⁰, —C(O)N(R²⁰)₂, and 4- to 6-membered heterocycle, wherein the 4- to 6-membered heterocycle is optionally substituted with one or more substituents selected from F, Cl, —OH, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from F, Cl. In some cases, each R⁸ is independently selected from F,

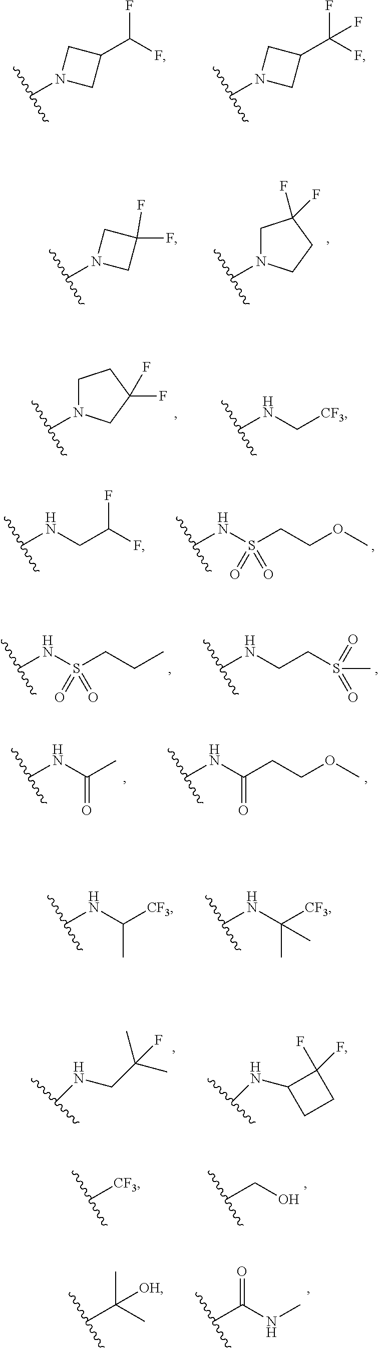

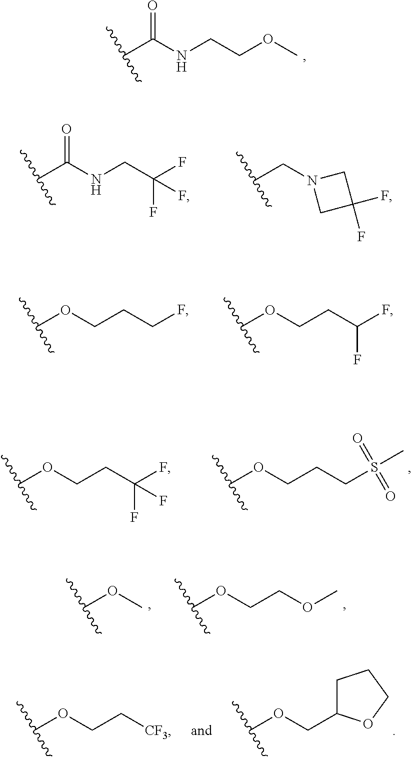

In some cases, R⁷ is

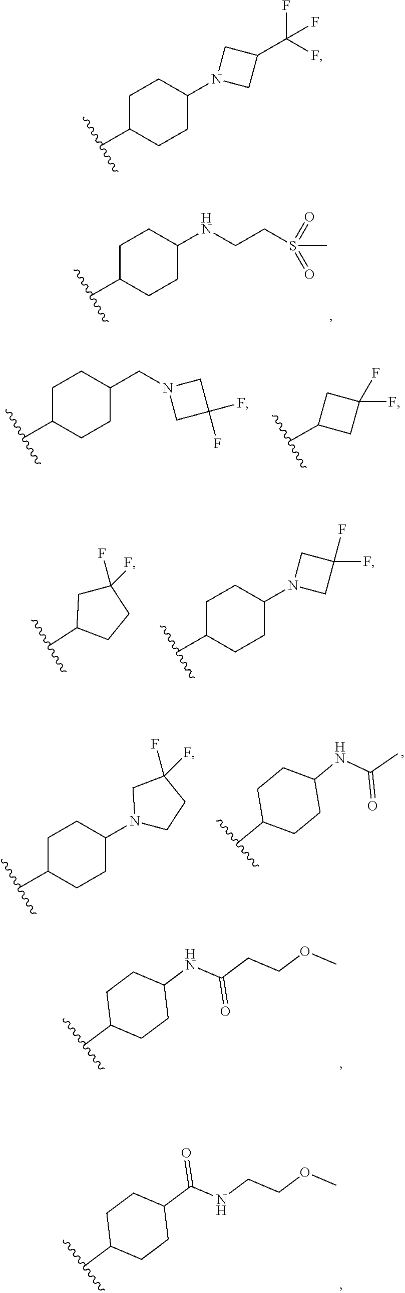

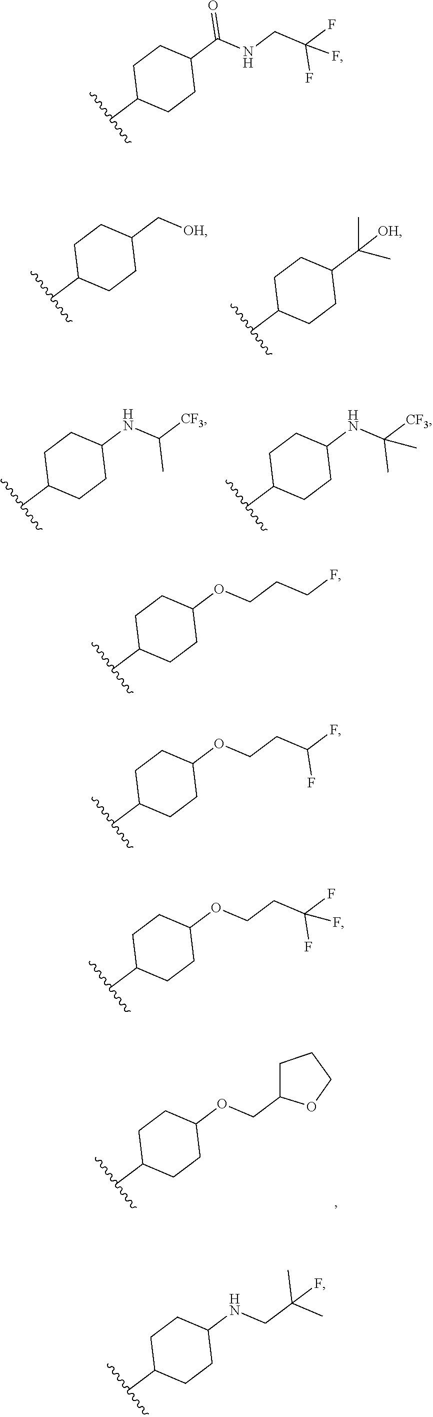

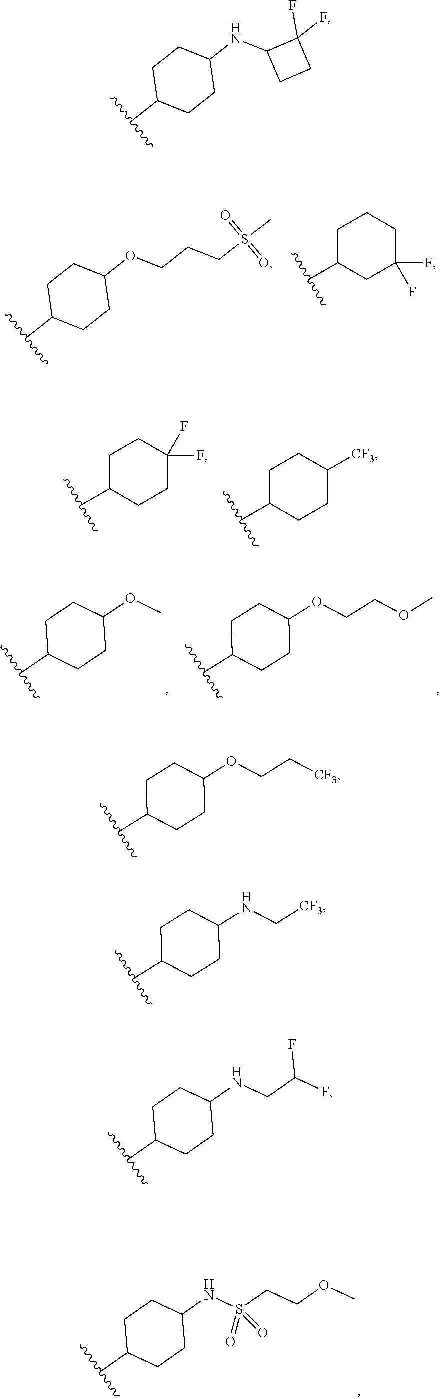

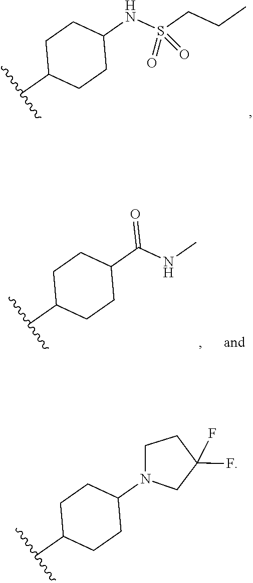 [SMILES: *C1CCC(NS(=O)(=O)CCC)CC1 ; *C1CCC(C(=O)NC)CC1 ; *C1CCC(N2CCC(F)(F)C2)CC1]

In some cases, each R⁸ is independently selected from halogen and C₁₋₆ haloalkyl. In some cases, each R⁸ is independently selected from fluorine and —CF₃. In some cases, In some cases, R⁷ is selected from

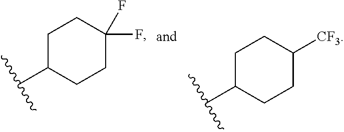 [SMILES: *C1CCC(F)(F)CC1 ; *C1CCC(C(F)(F)F)CC1]

In some cases, each R⁸ is independently selected from —OR²⁰. In some cases, R²⁰ is independently selected at each occurrence from C₁₋₆ alkyl, wherein the C₁₋₆ alkyl is optionally substituted with one or more substituents independently selected from halogen, and —O—C₁₋₁₀ alkyl. In some cases, each R⁸ is independently selected from —OCH₃,

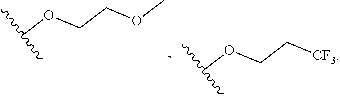

In some cases, In some cases, R⁷ is selected from

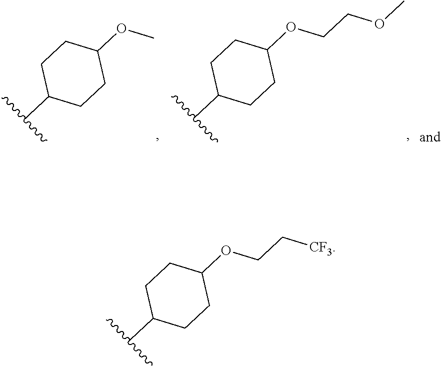

In some cases, each R⁸ is independently selected from —N(R²⁰)₂. In some cases, R²⁰ is independently selected at each occurrence from hydrogen and C₁₋₆ alkyl, wherein the C₁₋₆ alkyl is optionally substituted with one or more substituents independently selected from halogen. In some cases, each R²⁰ is different.

In some cases, each R⁸ is independently selected from

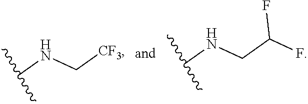 [SMILES: *NCC(F)(F)F ; *NCC(F)F]

In some cases, R⁷ is selected from

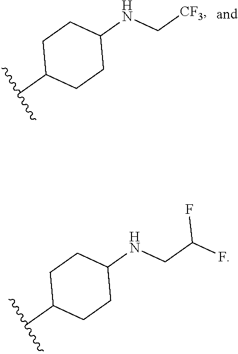 [SMILES: *C1CCC(NCC(F)(F)F)CC1 ; *C1CCC(NCC(F)F)CC1]

In some cases, each R⁸ is independently selected from —NR²⁰S(O)₂R²⁰. In some cases, each R⁸ is independently selected from —NHS(O)₂R²⁰. In some cases, R²⁰ is independently selected at each occurrence from C₁₋₆ alkyl, wherein the C₁₋₆ alkyl is optionally substituted with one or more substituents independently selected from —O—C₁₋₁₀ alkyl. In some cases, each R⁸ is independently selected from

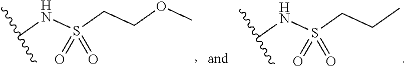

In some cases, R⁷ is selected from

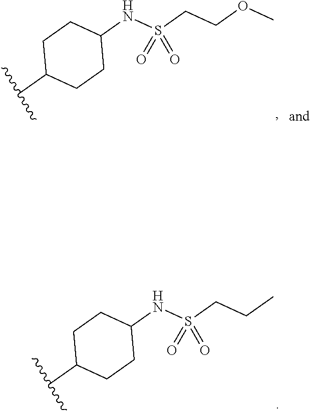 [SMILES: *C1CCC(NS(=O)(=O)CCOC)CC1 ; *C1CCC(NS(=O)(=O)CCC)CC1]

In some cases, each R⁸ is independently selected from —C(O)N(R²⁰)₂. In some cases, R²⁰ is independently selected at each occurrence from hydrogen and C₁₋₆ alkyl. In some cases, each R⁸ is independently selected from

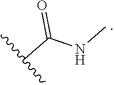 [SMILES: *C(=O)NC]

In some cases, R⁷ is

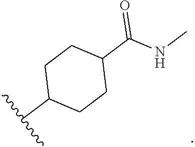 [SMILES: *C1CCC(C(=O)NC)CC1]

In some cases, each R⁸ is independently selected from 5- to 6-membered heterocycle, wherein the 5- to 6-membered heterocycle is optionally substituted with one or more substituents selected from halogen, C₁₋₁₀ alkyl, —C₁₋₁₀ haloalkyl, and —O—C₁₋₁₀ alkyl. In some cases, each R⁸ is independently selected from 5- to 6-membered heterocycle, wherein the 5- to 6-membered heterocycle is optionally substituted with one or more substituents selected from halogen. In some cases, each R⁸ is independently selected from

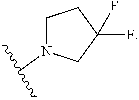 [SMILES: *N1CCC(F)(F)C1]

In some cases, R⁷ is

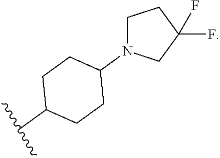 [SMILES: *C1CCC(N2CCC(F)(F)C2)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from 5- to 6-membered heterocycle, which is optionally substituted with one or more R⁸. In some cases, R⁷ is selected from 5-membered heterocycle, which is optionally substituted with one or more R⁸. In some cases, R⁷ is selected from 6-membered heterocycle, which is optionally substituted with one or more R⁸. In some cases, the heterocycle of R⁷ has at least one nitrogen atom. In some cases, the heterocycle of R⁷ has at least one oxygen atom. In some cases, the heterocycle of R⁷ has at least one sulfur atom. In some cases, each R⁸ is independently selected from halogen, —OR²⁰, —S(O)₂(R²⁰), —S)₂N(R²⁰)₂, —S(O)N(R²⁰)₂, —S(O)R²⁰(═NR²⁰), —NR²⁰S(O)₂R²⁰—C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle and 5- to 12-membered heterocycle. In some cases, each R⁸ is independently selected from halogen, —OR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —N(R²⁰)C(O)R²⁰ —N(R²⁰)C(O)N(R²⁰)₂, —N(R²⁰)C(O)OR²⁰, —C(O)OR²⁰, —OC(O)R²⁰, —OC(O)N(R²⁰) 2, —O, —CN, C₁₋₆ alkyl-N(R²⁰)₂, C₁₋₆ haloalkyl, C₁₋₆ alkyl, C₃-C₁₂ carbocycle and 5- to 12-membered heterocycle. In some cases, each R⁸ is independently selected from fluoro, chloro, —OR²⁰, —S(O)₂(R²⁰), —S)₂N(R²⁰)₂, —NR²⁰S(O)₂R²⁰, —C(O)N(R²⁰)₂, —N(R²⁰)C(O)R²⁰, —C(O)OR²⁰, —OC(O)R²⁰—OC(O)N(R²⁰) 2, =0, —CN, C₁₋₆ alkyl, C₃-C₁₂ carbocycle and 5- to 12-membered heterocycle. In some cases, each R⁸ is independently selected from fluoro, —OR²⁰, —S(O)₂(R²⁰), —S)₂N(R²⁰)₂, —S(O)N(R²⁰)₂, —S(O)R²⁰(═NR²⁰), ═O, and C₁₋₆ alkyl. In some cases, each R⁸ is independently selected from fluoro, —OR²⁰, —S(O)₂(R²⁰), —S)₂N(R²⁰)₂, —S(O)N(R²⁰)₂, —S(O)R²⁰(═NR²⁰), ═O. and C₁₋₄ alkyl. In some cases, each R⁸ is independently selected from fluoro, —S(O)₂(R²⁰), —S)₂N(R²⁰)₂, —S(O)N(R²⁰)₂, —S(O)R²⁰(═NR²⁰), methyl, ethyl, propyl, isopropyl, and ═O. In some cases, each R⁸ is independently selected from —S(O)₂(R²⁰), —S)₂N(R²⁰)₂, —S(O)N(R²⁰)₂, —S(O)R²⁰(═NR²⁰), and ═O. In some cases, each R⁸ is independently selected from —S(O)₂(R²⁰) and ═O. In some cases, each R⁸ is independently selected from fluoro, methyl, —S(O)₂(R²⁰) and ═O. In some cases, each R⁸ is —S(O)₂(R²⁰). In some cases, each R⁸ is ═O. In some cases, each R⁸ is methyl. In some cases, each R⁸ is fluoro. In some cases, R⁷ is selected from

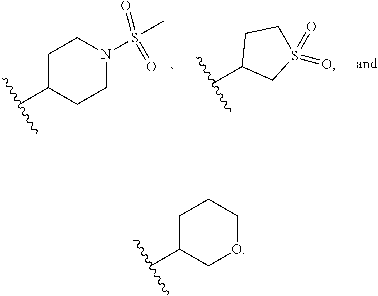 [SMILES: *C1CCN(S(C)(=O)=O)CC1 ; *C1CCS(=O)(=O)C1 ; *C1CCCOC1]

In some cases, R⁷ is

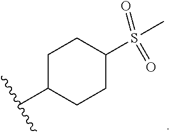 [SMILES: *C1CCC(S(C)(=O)=O)CC1]

In some cases, R⁷ is

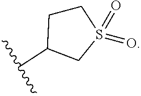 [SMILES: *C1CCS(=O)(=O)C1]

In some cases, R⁷ is

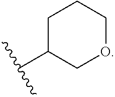 [SMILES: *C1CCCOC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is unsubstituted. In some cases, R⁷ is substituted.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is substituted with at least one R⁸ group. In some cases, R⁷ is substituted with at least two R⁸ groups. In some cases, R⁷ is substituted with at least three R⁸ groups. In some embodiments, for a compound or salt of Formula (I), R⁷ is substituted with no more than three R⁸ groups. In some cases, R⁷ is substituted with no more than two R⁸ groups. In some cases, R⁷ is substituted with no more than one R⁸ group. In some embodiments, for a compound or salt of Formula (I), R⁷ is substituted with three R⁸ groups. In some cases, R⁷ is substituted with two R⁸ groups. In some cases, R⁷ is substituted with one R⁸ group.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 8-membered heterocycle. In some cases, the saturated 4- to 8-membered heterocycle is optionally substituted with one or more R⁸. In some cases, R⁷ is selected from an optionally substituted saturated 5-membered heterocycle. In some cases, the 5-membered heterocycle is optionally substituted with one or more R⁸. In some cases, R⁷ is selected from an optionally substituted saturated 6-membered heterocycle. In some cases, the 6-membered heterocycle is optionally substituted with one or more R⁸. In some cases, R⁷ is selected from an optionally substituted saturated 4- to 6-membered heterocycle. In some cases, R⁷ is selected from an optionally substituted saturated 5- to 6-membered heterocycle. In some cases, R⁷ is selected from an optionally substituted saturated 4-membered heterocycle. In some cases, R⁷ is selected from an optionally substituted saturated 5-membered heterocycle. In some cases, R⁷ is selected from an optionally substituted saturated 6-membered heterocycle. In some cases, R⁷ is selected from an optionally substituted saturated 4- to 6-membered heterocycle, wherein the saturated 4- to 6-membered heterocycle has at least one heteroatom selected from oxygen, nitrogen, and sulfur. In some cases, the saturated 4- to 6-membered heterocycle has at most one heteroatom. In some cases, R⁷ is selected from

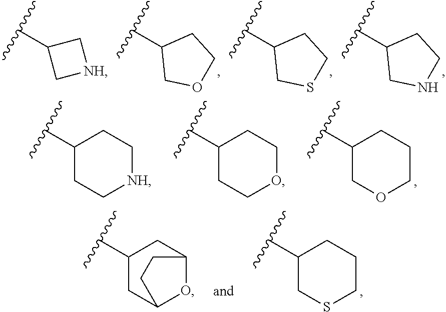

wherein each is optionally substituted with one or more substituents independently selected from R⁸. In some cases, R⁷ is selected from

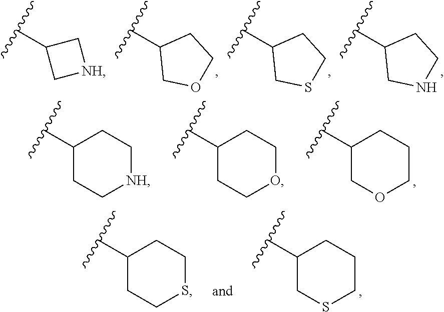

wherein each is optionally substituted with one or more substituents independently selected from R⁸. In some cases, R⁷ is selected from

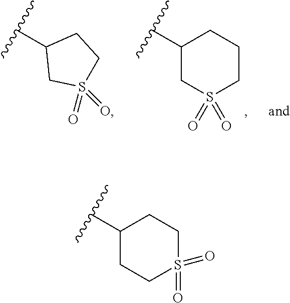 [SMILES: *C1CCS(=O)(=O)C1 ; *C1CCCS(=O)(=O)C1 ; *C1CCS(=O)(=O)CC1]

wherein each is optionally substituted with one or more substituents independently selected from R⁸. In some cases, R⁷ is selected from

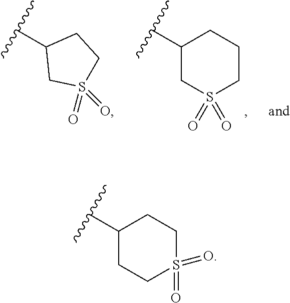 [SMILES: *C1CCS(=O)(=O)C1 ; *C1CCCS(=O)(=O)C1 ; *C1CCS(=O)(=O)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 8-membered heterocycle. In some cases, the saturated 4- to 8-membered heterocycle is optionally substituted with one or more R⁸. In some cases, R⁷ is selected from an optionally substituted saturated 5-membered heterocycle. In some cases, the 5-membered heterocycle is optionally substituted with one or more R⁸. In some cases, R⁷ is selected from an optionally substituted saturated 6-membered heterocycle. In some cases, the 6-membered heterocycle is optionally substituted with one or more R⁸. In some cases, R⁷ is selected from an optionally substituted saturated 4- to 6-membered heterocycle. In some cases, R⁷ is selected from an optionally substituted saturated 5- to 6-membered heterocycle. In some cases, R⁷ is selected from an optionally substituted saturated 4-membered heterocycle. In some cases, R⁷ is selected from an optionally substituted saturated 5-membered heterocycle. In some cases, R⁷ is selected from an optionally substituted saturated 6-membered heterocycle. In some cases, R⁷ is selected from an optionally substituted saturated 4- to 6-membered heterocycle, wherein the saturated 4- to 6-membered heterocycle has at least one heteroatom selected from oxygen, nitrogen, and sulfur. In some cases, the saturated 4- to 6-membered heterocycle has at most one heteroatom. In some cases, R⁷ is selected from

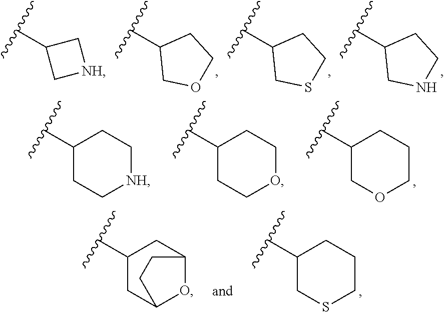

wherein each is optionally substituted with one or more substituents independently selected from R⁸. In some cases, R⁷ is selected from

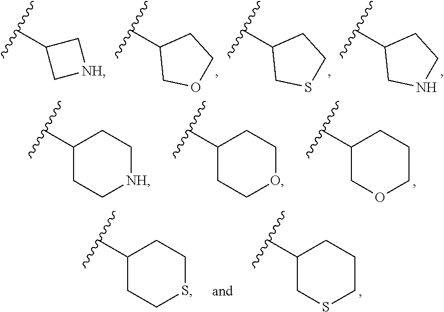

wherein each is optionally substituted with one or more substituents independently selected from R⁸. In some cases, R⁷ is selected from

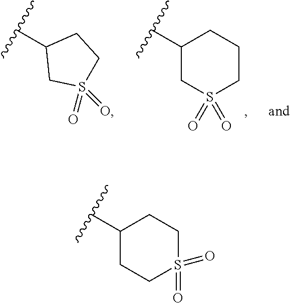 [SMILES: *C1CCS(=O)(=O)C1 ; *C1CCCS(=O)(=O)C1 ; *C1CCS(=O)(=O)CC1]

wherein each is optionally substituted with one or more substituents independently selected from R⁸. In some cases, R⁷ is selected from

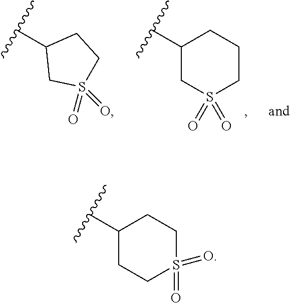 [SMILES: *C1CCS(=O)(=O)C1 ; *C1CCCS(=O)(=O)C1 ; *C1CCS(=O)(=O)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 6-membered heterocycle, wherein the optionally substituted saturated 4- to 6-membered heterocycle contains at least one nitrogen atom. In some cases, the optionally substituted saturated 4- to 6-membered heterocycle contains only 1 nitrogen atom. In some cases, the optionally substituted saturated 4- to 6-membered heterocycle contains at most 1 nitrogen atom. In some cases, R⁷ is selected from

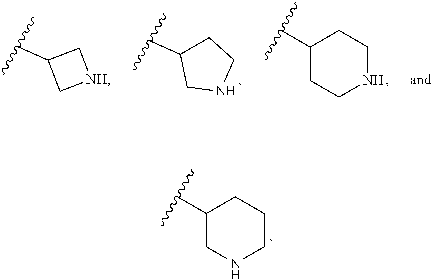 [SMILES: *C1CNC1 ; *C1CCNC1 ; *C1CCNCC1 ; *C1CCCNC1]

wherein each is optionally substituted with one or more substituents independently selected from R⁸. In some cases, R⁷ is selected from

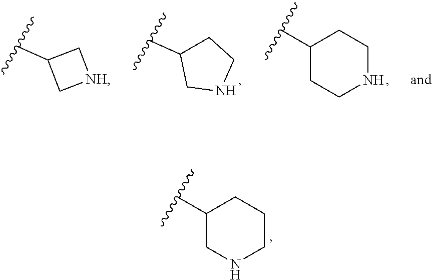 [SMILES: *C1CNC1 ; *C1CCNC1 ; *C1CCNCC1 ; *C1CCCNC1]

wherein each is optionally substituted with one or more substituents selected from halogen, —C(O)R²⁰ and —S(O)₂(R²⁰). In some cases, R⁷ is selected from

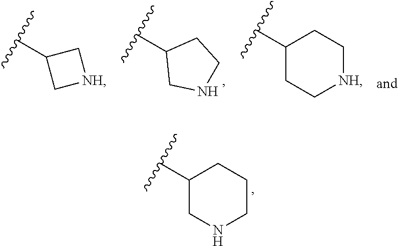 [SMILES: *C1CNC1 ; *C1CCNC1 ; *C1CCNCC1 ; *C1CCCNC1]

wherein each is substituted with —S(O)₂(R²⁰). In some cases, R⁷ is selected from

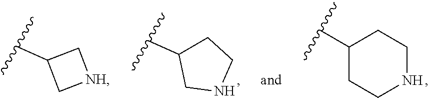 [SMILES: *C1CNC1 ; *C1CCNC1 ; *C1CCNCC1]

wherein each is substituted with —S(O)₂(R²⁰). In some cases, each R²⁰ is independently selected at each occurrence from hydrogen; C₁₋₆ alkyl and C₃₋₁₂ carbocycle, wherein the C₁₋₆ alkyl is optionally substituted with one or more-O—C₁₋₁₀ alkyl. In some cases, each R²⁰ is independently selected at each occurrence from hydrogen; C₁₋₆ alkyl and C₃₋₆ carbocycle. In some cases, each R²⁰ is independently selected at each occurrence from C₁₋₆ alkyl and C₃₋₆ carbocycle. In some cases, each R²⁰ is independently selected at each occurrence from C₁₋₆ alkyl. In some cases, each R²⁰ is independently selected at each occurrence from C₃₋₆ carbocycle. In some cases, R⁷ is selected from

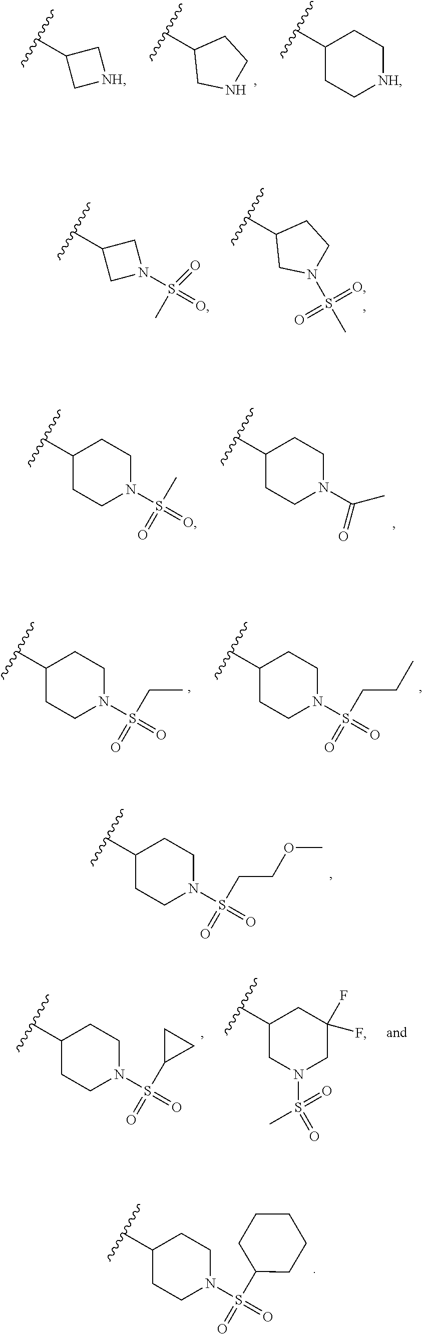

In some cases, R⁷ is selected from

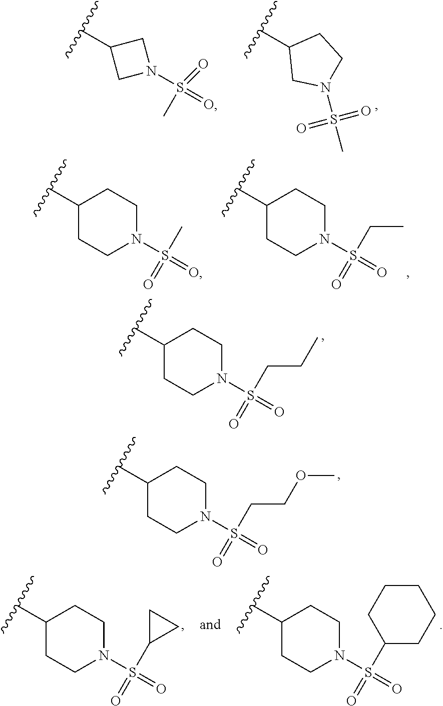

In some cases, R⁷ is selected from

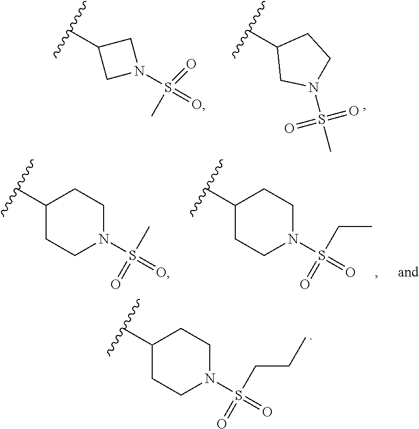

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 6-membered heterocycle, wherein the optionally substituted saturated 4- to 6-membered heterocycle contains at least one nitrogen atom. In some cases, R⁷ is selected from

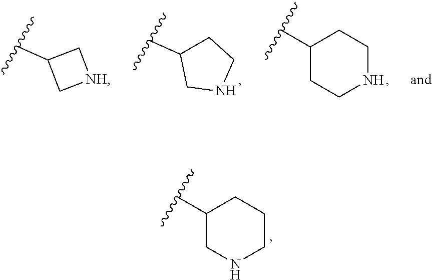 [SMILES: *C1CNC1 ; *C1CCNC1 ; *C1CCNCC1 ; *C1CCCNC1]

wherein each is optionally substituted with one or more substituents independently selected from R⁸. In some cases, R⁷ is a beta lactam 4- to 8-membered saturated heterocyle, wherein each is optionally substituted with one or more substituents independently selected from R⁸. In some cases, R⁷ is selected from

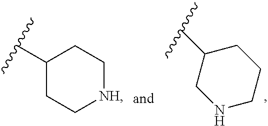 [SMILES: *C1CCNCC1 ; *C1CCCNC1]

wherein each is optionally substituted with one or more substituents independently selected from R⁸. In some cases, R⁷ is

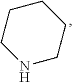

which is optionally substituted with one or more substituents independently selected from R⁸. In some cases, R⁷ is selected from

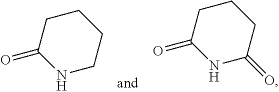 [SMILES: O=C1CCCCN1 ; O=C1CCCC(=O)N1]

each of which is optionally substituted with one or more substituents independently selected from R⁸. In some cases, R⁷ is

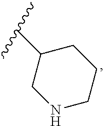 [SMILES: *C1CCCNC1 ; ","]

which is optionally substituted with one or more substituents independently selected from R⁸. In some cases, each R⁸ is selected from C₁₋₆ alkyl, —O, and saturated 4- to 8-membered heterocycle. In some cases, each R⁸ is selected from C₁₋₆ alkyl and ═O. In some cases, each R⁸ is selected from C₁₋₆ alkyl and ═O. In some cases, R⁷ is selected from

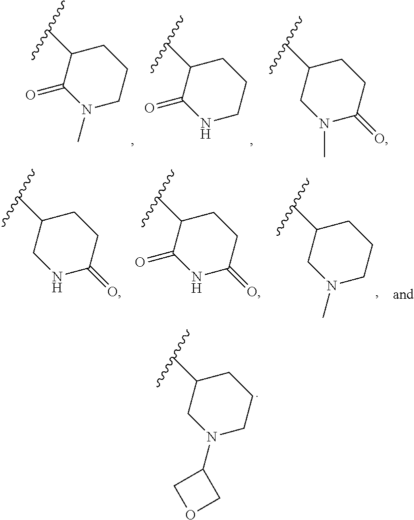

In some cases, R⁷ is selected from

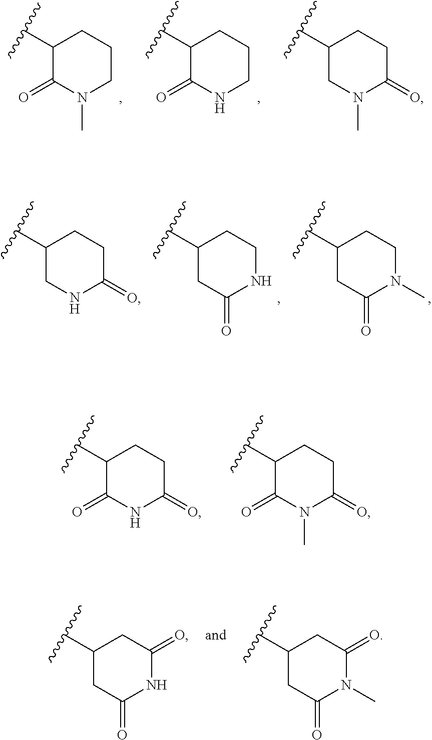

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an saturated 4- to 6-membered heterocycle, wherein the saturated 4- to 6-membered heterocycle contains at least one nitrogen atom and is substituted with at least one oxo, and is further optionally substituted with one or more R⁸. In some cases, R⁷ is selected from

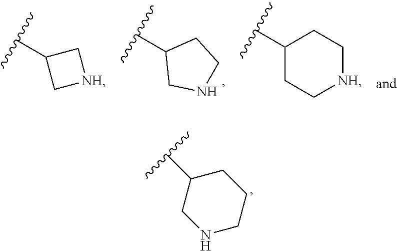 [SMILES: *C1CNC1 ; *C1CCNC1 ; *C1CCNCC1 ; *C1CCCNC1]

wherein each is substituted with at least one oxo and one or more substituents independently selected from R⁸. In some cases, R⁷ is selected from

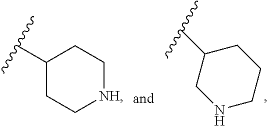 [SMILES: *C1CCNCC1 ; *C1CCCNC1]

wherein each is substituted with at least one oxo and further optionally substituted with one or more substituents independently selected from R⁸. In some cases, R⁷ is

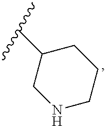 [SMILES: *C1CCCNC1 ; ","]

which is substituted with at least one oxo and further optionally substituted with one or more substituents independently selected from R⁸. In some cases, each R⁸ is selected from C₁₋₆ alkyl, ═O, and saturated 4- to 8-membered heterocycle. In some cases, each R⁸ is selected from C₁₋₆ alkyl and ═O. In some cases, each R⁸ is selected from C₁₋₆ alkyl. In some cases, each R⁸ is selected from ═O. In some cases, R⁷ is selected from

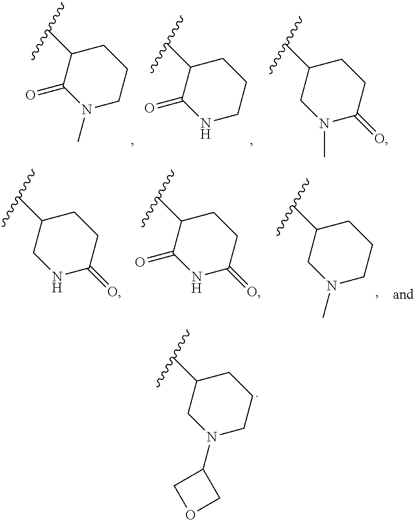

In some cases, R⁷ is selected from

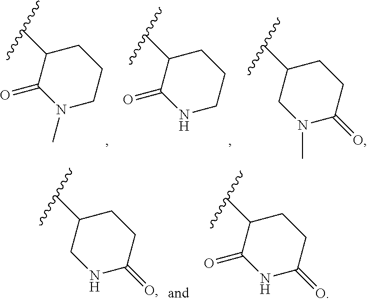

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 7- to 8-membered spiro heterocycle. In some cases, the spiro heterocycle has at least one nitrogen atom. In some cases, the spiro heterocycle has at least one oxygen atom. In some cases, R⁷ is selected from

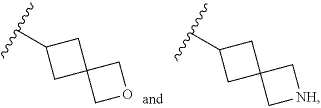 [SMILES: *C1CC2(COC2)C1 ; *C1CC2(CNC2)C1]

each of which is optionally substituted with one or more substituents independently selected from R⁸. In some cases, R⁷ is selected from

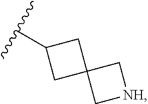 [SMILES: *C1CC2(CNC2)C1]

which is optionally substituted with one or more substituents independently selected from R⁸. In some cases, each R⁸ is independently selected from halogen, —OR²⁰, —SR²⁰, —N(R²⁰)₂, —NO₂, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²⁰)₂, —S(O)₂(R²⁰), —S(O)(R²⁰), —S(O)₂(NR₂²⁰), —S(O)(NR²⁰)R²⁰, and —S(O)(NR²⁰)N(R²⁰)₂. In some cases, each R⁸ is independently selected from C₁₋₆ alkyl, —S(O)₂(R²⁰), —S(O)(R²⁰), —S(O)₂(NR₂²⁰), —S(O)(NR²⁰)R²⁰, and —S(O)(NR²⁰)N(R²⁰)₂. In some cases, each R⁸ is independently selected from C₁₋₆ alkyl and —S(O)₂(R²⁰). In some cases, R⁷ is

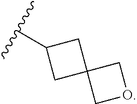 [SMILES: *C1CC2(COC2)C1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 6-membered heterocycle, wherein the optionally substituted saturated 4- to 6-membered heterocycle contains at least one nitrogen atom and is substituted with at least one halogen, and least one-S(O)₂(R²⁰). In some cases, R⁷ is

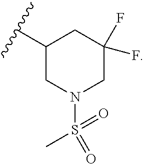 [SMILES: *C1CN(S(C)(=O)=O)CC(F)(F)C1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 6-membered heterocycle, and C₆ cycloalkyl substituted with one or more R⁸. In some cases, the saturated 6-membered heterocycle contains 1 nitrogen atom. In some cases, the saturated 6-membered heterocycle contains only 1 nitrogen atom and no further heteroatoms. In some cases, R⁷ is selected from

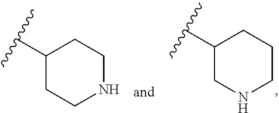 [SMILES: *C1CCNCC1 ; *C1CCCNC1]

each of which is optionally substituted with one or more substituents independently selected from R⁸; and C₆ cycloalkyl substituted with one or more R⁸. In some cases, R⁷ is selected from

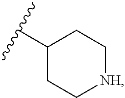 [SMILES: *C1CCNCC1]

which is optionally substituted with —C(O)R²⁰ and —S(O)₂(R²⁰); and C₆ cycloalkyl substituted with one or more halogen. In some cases, R⁷ is selected from

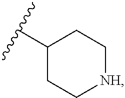 [SMILES: *C1CCNCC1]

which is optionally substituted with —S(O)₂(R²⁰); and C₆ cycloalkyl substituted with one or more halogen. In some cases, R⁷ is selected from

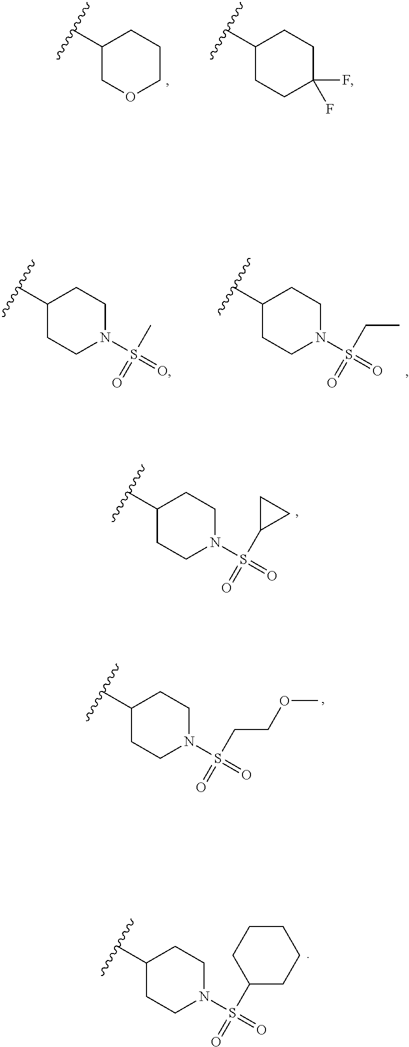

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 6-membered heterocycle, and optionally substituted C₄ cycloalkyl, each of which is optionally substituted with one or more substituents independently selected from halogen, C₁₋₆ haloalkyl, C₁₋₆ alkoxyalkyl, —S(O)₂(R²⁰), —S(O)(R²⁰), —S(O)₂(NR₂²⁰), —S(O)(NR²⁰)R²⁰, and —S(O)(NR²⁰)N(R²⁰)₂. In some cases, R⁷ is selected from an optionally substituted saturated 6-membered heterocycle, and unsubstituted C₄ cycloalkyl, wherein the saturated 6-membered heterocycle is optionally substituted with one or more substituents independently selected from —S(O)₂(R²⁰). In some cases, R⁷ is selected from unsubstituted C₄ cycloalkyl,

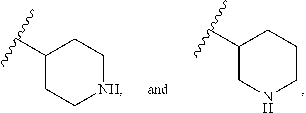 [SMILES: *C1CCNCC1 ; *C1CCCNC1]

each of which is optionally substituted with —S(O)₂(R²⁰) and halogen. In some cases, R⁷ is selected from unsubstituted C₄ cycloalkyl,

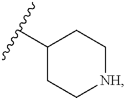 [SMILES: *C1CCNCC1]

each of which is optionally substituted with —S(O)₂(R²⁰). In some cases, R⁷ is selected from

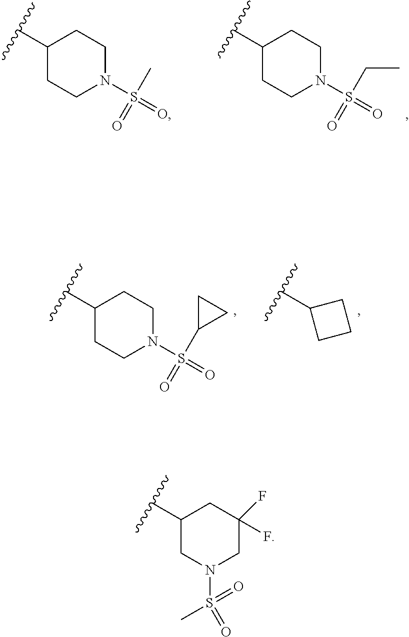 [SMILES: *C1CCN(S(C)(=O)=O)CC1 ; *C1CCN(S(=O)(=O)CC)CC1 ; *C1CCN(S(=O)(=O)C2CC2)CC1 ; *C1CCC1 ; *C1CN(S(C)(=O)=O)CC(F)(F)C1]

In some cases, R⁷ is selected from

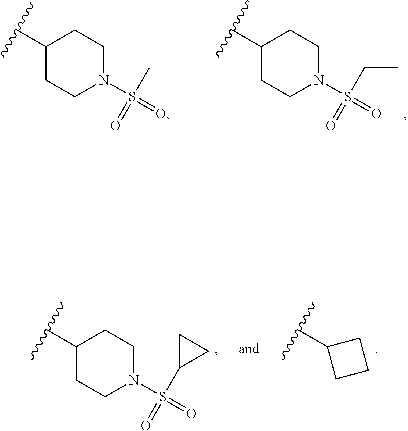 [SMILES: *C1CCN(S(C)(=O)=O)CC1 ; *C1CCN(S(=O)(=O)CC)CC1 ; *C1CCN(S(=O)(=O)C2CC2)CC1 ; *C1CCC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 8-membered heterocycle, wherein the optionally substituted saturated 4- to 8-membered heterocycle contains at least one oxygen atom. In some cases, the optionally substituted saturated 4- to 8-membered heterocycle contains only 1 oxygen atom. In some cases, the optionally substituted saturated 4- to 6-membered heterocycle contains at most 1 oxygen atom. In some cases, R⁷ is selected from

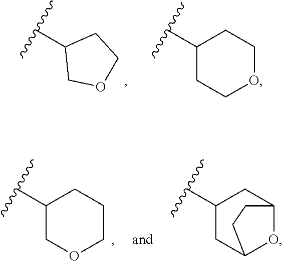 [SMILES: *C1CCOC1 ; *C1CCOCC1 ; *C1CCCOC1 ; *C1CC2CCC(C1)O2]

wherein each is optionally substituted with one or more substituents independently selected from R⁸. In some cases, R⁷ is selected from an optionally substituted saturated 4- to 6-membered heterocycle, wherein the optionally substituted saturated 4- to 6-membered heterocycle contains at least one oxygen atom. In some cases, R⁷ is selected from

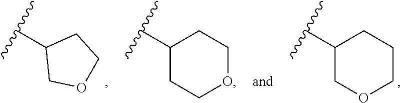 [SMILES: *C1CCOC1 ; *C1CCOCC1 ; *C1CCCOC1]

each is optionally substituted with one or more substituents independently selected from R⁸. In some cases, R⁷ is

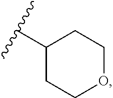 [SMILES: *C1CCOCC1]

which is optionally substituted with one or more substituents independently selected from R⁸. In some cases, R⁷ is

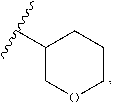 [SMILES: *C1CCCOC1]

which is optionally substituted with one or more substituents independently selected from R⁸.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is an optionally substituted 6-membered saturated heterocycle containing one oxygen atom and no further heteroatoms. In some cases, the heterocycle is tetrahydropyran. In some cases, each R⁸ is independently selected from halogen, —OR²⁰, —SR²⁰, —N(R²⁰)₂, —NO₂, —O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)₂(NR₂²⁰), —S(O)(NR²⁰)R²⁰, and —S(O)(NR²⁰)N(R²⁰)₂. In some cases, each R⁸ is independently selected from halogen, —OR²⁰, ═O, —CN, C₁₋₆ aminoalkyl, C₁₋₆ hydroxyalkyl, C₁₋₆ cyanoalkyl, C₁₋₆ haloalkyl, C₁₋₆ alkoxyalkyl, and C₁₋₆ alkyl. In some cases, each R⁸ is independently selected from halogen and C₁₋₆ alkyl. In some cases, the heterocycle is unsubstituted. In some cases, the heterocycle is substituted. In some cases, R⁷ is selected from

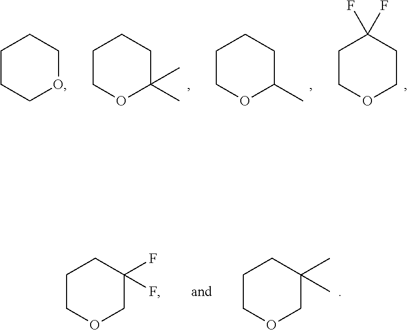

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 8-membered heterocycle, wherein the optionally substituted saturated 8-membered heterocycle contains at least one oxygen atom. In some cases, the saturated 8-membered heterocycle is a bridged heterocycle. In some cases, R⁷ is

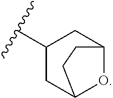 [SMILES: *C1CC2CCC(C1)O2]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted saturated 4- to 6-membered heterocycle, wherein the optionally substituted saturated 4- to 6-membered heterocycle contains at least one sulfur atom. In some cases, the optionally substituted saturated 4- to 6-membered heterocycle contains only 1 sulfur atom. In some cases, the optionally substituted saturated 4- to 6-membered heterocycle contains at most 1 sulfur atom. In some cases, R⁷ is selected from

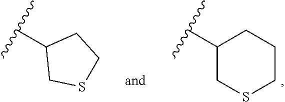 [SMILES: *C1CCSC1 ; *C1CCCSC1]

wherein each is optionally substituted with one or more substituents independently selected from R⁸. In some cases, R⁷ is selected from

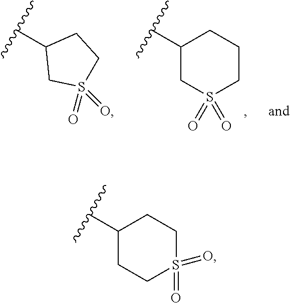 [SMILES: *C1CCS(=O)(=O)C1 ; *C1CCCS(=O)(=O)C1 ; *C1CCS(=O)(=O)CC1]

wherein each is optionally substituted with one or more substituents independently selected from R⁸. In some cases, R⁷ is selected from

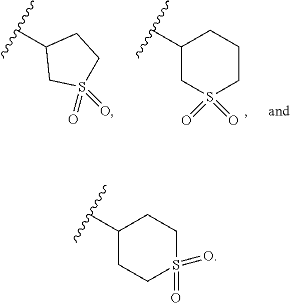 [SMILES: *C1CCS(=O)(=O)C1 ; *C1CCCS(=O)(=O)C1 ; *C1CCS(=O)(=O)CC1]

In some cases, R⁷ is selected from

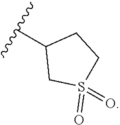 [SMILES: *C1CCS(=O)(=O)C1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸ is independently selected from halogen, —OR²⁰, —O, C₁₋₆ hydroxyalkyl, C₁₋₆ haloalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰) R²⁰—S(O)₂(NR₂²⁰), and —S(O)(NR²⁰)N(R²⁰)₂. In some cases, each R⁸ is independently selected from halogen, —OR²⁰, ═O, C₁₋₆ hydroxyalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰)R²⁰, and —S(O)(NR²⁰)N(R²⁰)₂. In some cases, each R⁸ is independently selected from —C(O)R²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰)R²⁰, and —S(O)(NR²⁰)N(R²⁰)₂. In some cases, each R⁸ is independently selected from —C(O)R²⁰, —C(O)N(R²⁰)₂, —S(O)₂(R²⁰), and —S(O)(NR²⁰) R²⁰. In some cases, each R⁸ is independently selected from halogen, —OR²⁰, C₁₋₆ hydroxyalkyl, C₁₋₆ haloalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰) R²⁰ and —S(O)(NR²⁰)N(R²⁰)₂. In some cases, each R⁸ is independently selected from halogen, —OR²⁰ ═O, C₁₋₆ hydroxyalkyl, C₁ haloalkyl, C₃₋₆ haloalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰) R²⁰ and —S(O)(NR²⁰)N(R²⁰)₂. In some cases, R⁸ is selected from halogen, ═O, C₁₋₆ hydroxyalkyl, C₁ haloalkyl, C₁₋₆ alkyl, —C(O)R²⁰, —S(O)₂(R²⁰), —S(O)(R²⁰), —S(O)(NR²⁰)R²⁰, and —S(O)(NR²⁰)N(R²⁰)₂. In some cases, R⁸ is selected from ═O, —C(O)R²⁰, —S(O)₂(R²⁰), —S(O)(R²⁰), —S(O)(NR²⁰)R²⁰, and —S(O)(N R²⁰)N(R²⁰)₂. In some cases, R⁸ is selected from —S(O)₂(R²⁰), —S(O)₂(NR₂²⁰), —S(O)(NR²⁰)R²⁰, and —S(O)(NR²⁰)N(R²⁰)₂. In some cases, R⁸ is selected from —S(O)₂(R²⁰). In some cases, R⁸ is selected from —C(O)N(R²⁰)₂, —S(O)₂(R²⁰), —S(O)(NR²⁰)R²⁰, and —C(O)R²⁰. In some cases, R⁸ is selected from —C(O)N(R²⁰)₂, —S(O)₂(R²⁰), and —S(O)(NR²⁰) R²⁰. In some cases, R⁸ is —S(O)₂(NR₂²⁰). In some cases, R⁸ is selected from —S(O)₂(R²⁰) and —C(O)R²⁰ In some cases, R⁸ is —C(O)N(R²⁰)₂. In some cases, R⁸ is —S(O)₂(R²⁰). In some cases, R⁸ is —S(O)(NR²⁰) R²⁰. In some cases, R⁸ is selected from halogen, —OR²⁰, and ═O. In some cases, R⁸ is selected from C₁₋₆ hydroxyalkyl, C₁₋₆ haloalkyl, and C₁₋₆ alkyl. In some cases, R⁸ is selected from halogen. In some cases, each R²⁰ is independently selected at each occurrence from hydrogen; C₁₋₆ alkyl, C₃₋₁₂ carbocycle, and 3- to 12-membered heterocycle. In some cases, each R²⁰ is independently selected at each occurrence from hydrogen: C₁₋₆ alkyl and C₃₋₁₂ carbocycle, wherein the C₁₋₆ alkyl is optionally substituted with one or more-O—C₁₋₁₀ alkyl. In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, each R²⁰ is independently selected at each occurrence from hydrogen and C₁₋₆ alkyl. In some cases, each R²⁰ is independently selected at each occurrence from hydrogen, C₁₋₆ alkyl, C₃₋₁₂ carbocycle, and 3- to 12-membered heterocycle. In some cases, each R²⁰ is independently selected at each occurrence from hydrogen and C₁₋₆ alkyl. In some cases, R⁷ is selected from

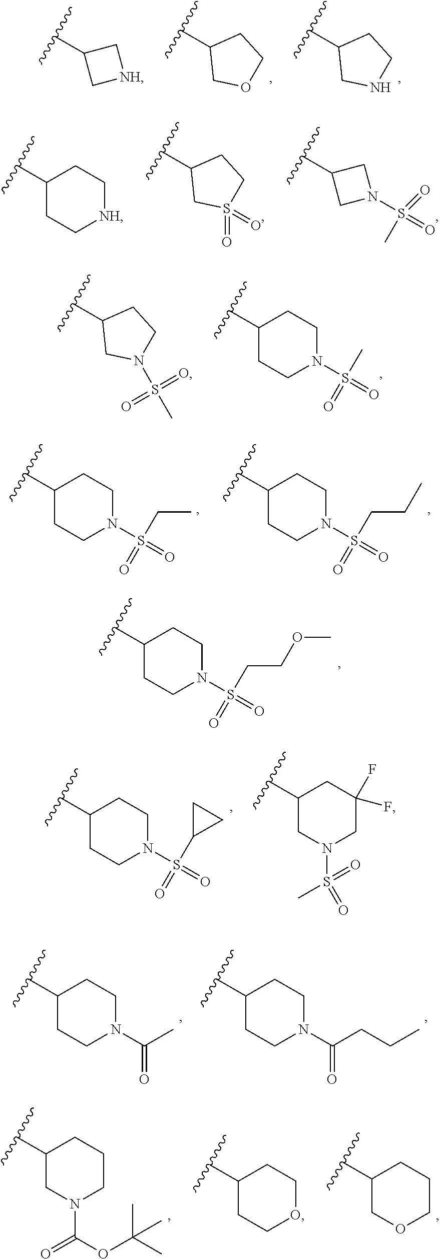

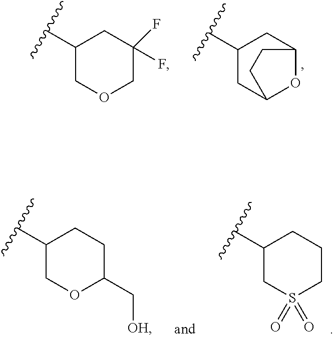 [SMILES: *C1COCC(F)(F)C1 ; *C1CC2CCC(C1)O2 ; *C1CCC(CO)OC1 ; *C1CCCS(=O)(=O)C1]

In some cases, R⁷ is selected from

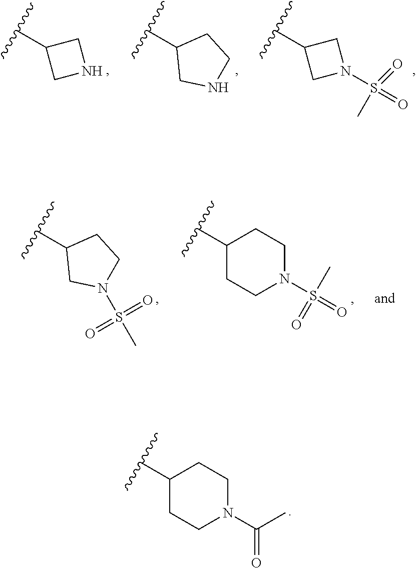

In some cases, R⁷ is selected from

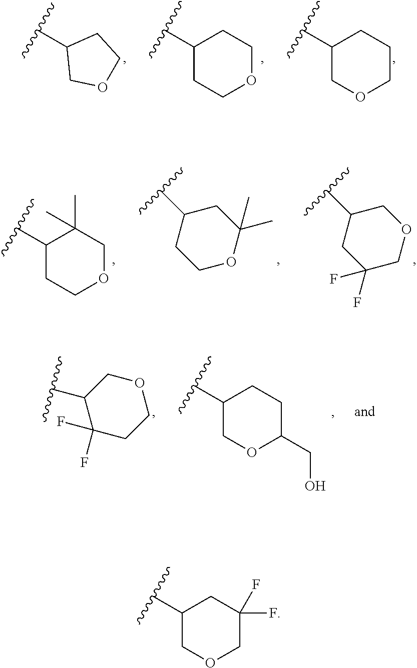

In some cases, R⁷ is selected from,

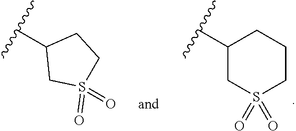 [SMILES: *C1CCS(=O)(=O)C1 ; "and" ; *C1CCCS(=O)(=O)C1 ; "."]

In some cases, R⁷ is selected from

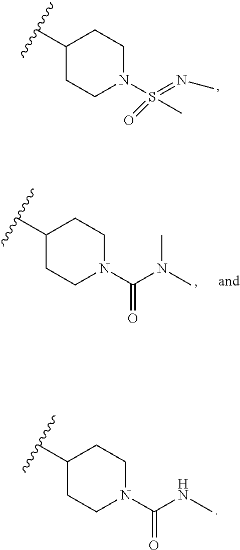 [SMILES: *C1CCN(S(C)(=O)=NC)CC1 ; *C1CCN(C(=O)N(C)C)CC1 ; *C1CCN(C(=O)NC)CC1]

In some cases, R⁷ is selected from

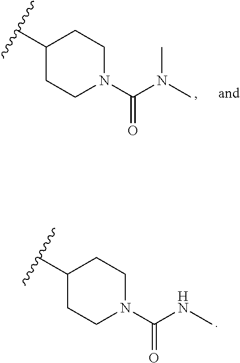 [SMILES: *C1CCN(C(=O)N(C)C)CC1 ; *C1CCN(C(=O)NC)CC1]

In some cases, R⁷ is selected from

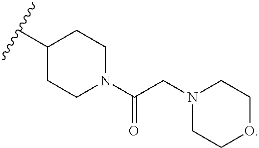 [SMILES: *C1CCN(C(=O)CN2CCOCC2)CC1]

In some cases, R⁷ is selected from

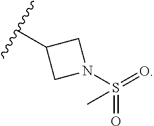 [SMILES: *C1CN(S(C)(=O)=O)C1]

In some cases, R⁷ is selected from

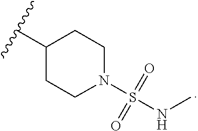 [SMILES: *C1CCN(S(=O)(=O)NC)CC1]

In some cases, R⁷ is selected from

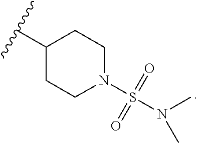 [SMILES: *C1CCN(S(=O)(=O)N(C)C)CC1]

In some cases, R⁷ is selected from

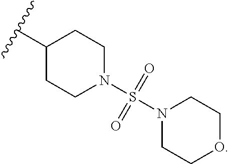 [SMILES: *C1CCN(S(=O)(=O)N2CCOCC2)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from 5- to 10-membered heterocycle, each of which are optionally substituted with one or more R⁸. In some cases, R⁷ is selected from

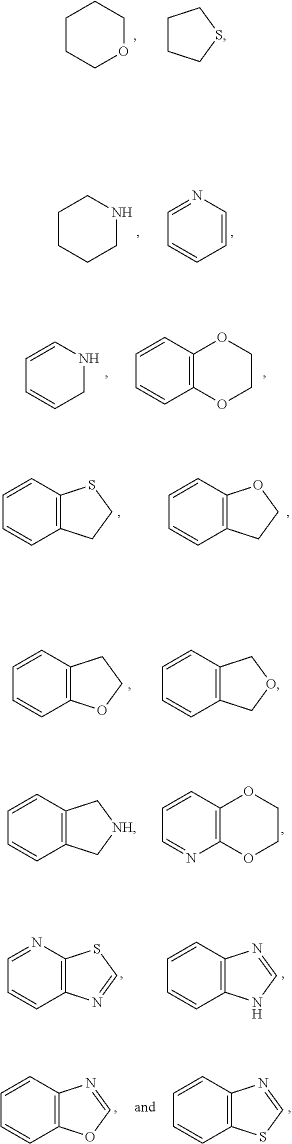

each of which are optionally substituted with one or more R⁸. In some cases, the heterocycle has one oxygen atom. In some cases, the heterocycle has one nitrogen atom. In some cases, the heterocycle has only one heteroatom. In some cases, the heterocycle has only two heteroatoms. In some cases, the heterocycle has only three heteroatoms. In some cases, the heterocycle has one sulfur atom. In some cases, each R⁸ is independently selected from halogen, —OR²⁰, —S(O)₂(R²⁰), —C(O)OR²⁰, ═O, C₁₋₆ alkyl, and C₁₋₆ haloalkyl. In some cases, each R⁸ is independently selected from —O, —S(O)₂(C₁₋₆ alkyl), —CF₃, —CH₃, —C(O)OC₁₋₆ alkyl, —OMe, and

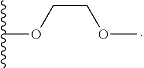 [SMILES: *OCCOC]

In some cases, R⁷ is selected from

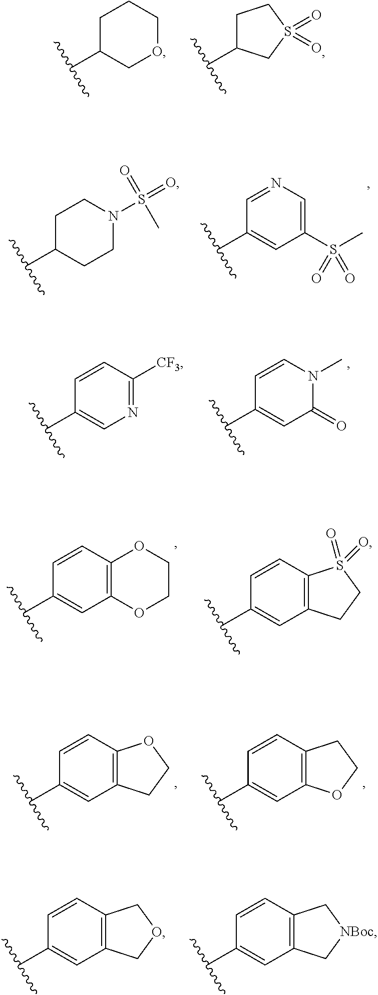

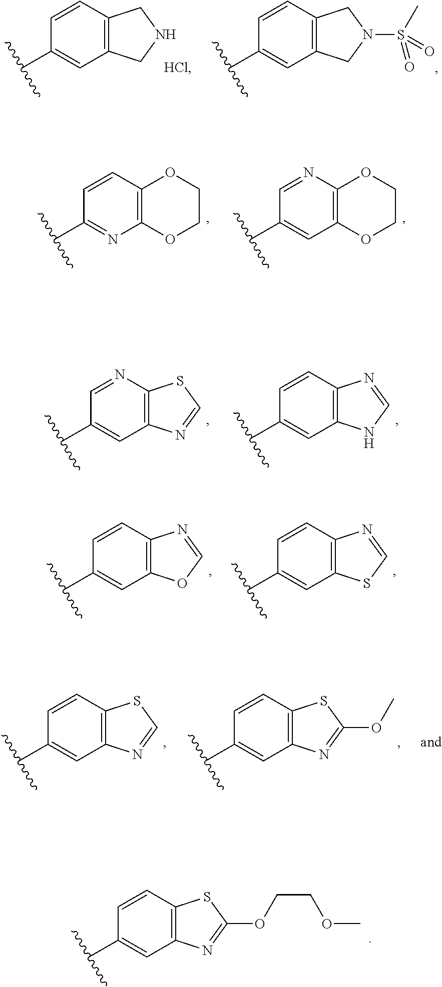

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from 5- to 10-membered heterocycle, each of which are optionally substituted with one or more R⁸. In some cases, R⁷ is selected from

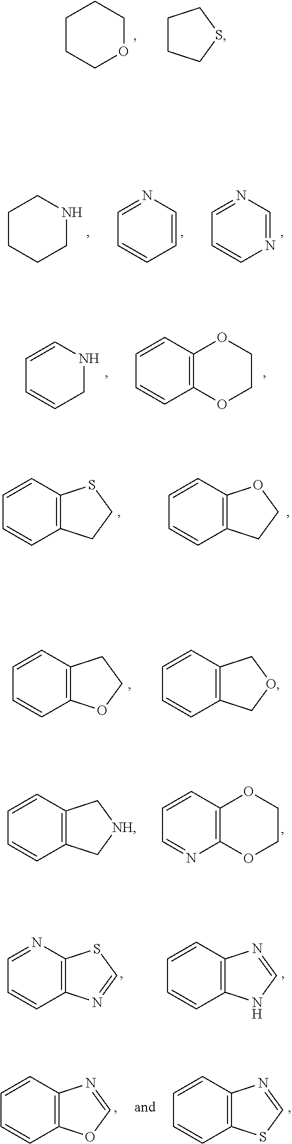

each of which are optionally substituted with one or more R⁸. In some cases, R⁷ is selected from

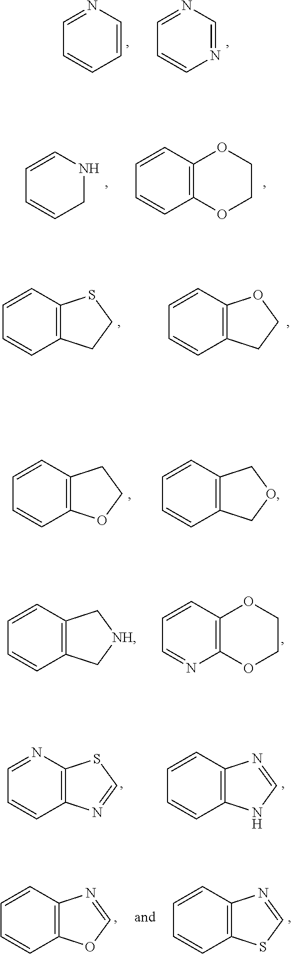

each of which are optionally substituted with one or more R⁸. In some cases, R⁷ is selected from

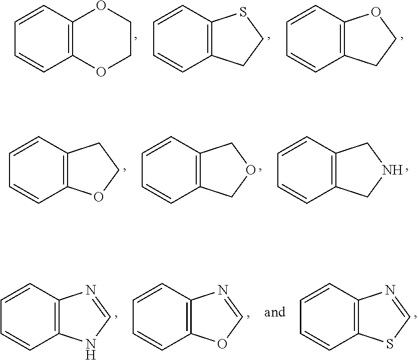

each of which are optionally substituted with one or more R⁸. In some cases, R⁷ is selected from

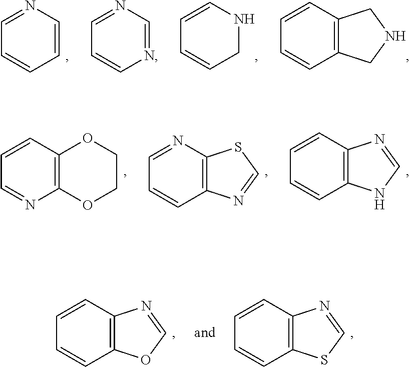

each of which are optionally substituted with one or more R⁸. In some cases, R⁷ is selected from

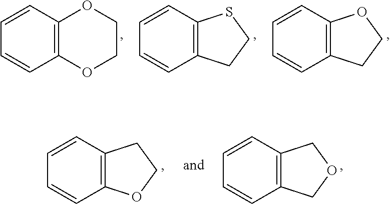

each of which are optionally substituted with one or more R⁸. In some cases, the heterocycle has one oxygen atom. In some cases, the heterocycle has one nitrogen atom. In some cases, the heterocycle has only one heteroatom. In some cases, the heterocycle has only two heteroatoms. In some cases, the heterocycle has only three heteroatoms. In some cases, the heterocycle has one sulfur atom. In some cases, each R⁸ is independently selected from halogen, —OR²⁰, —N(R²⁰)₂, —S(O)₂(R²⁰), —C(O)OR²⁰, ═O, C₁₋₆ alkyl, C₁₋₆ haloalkyl, and 4- to 6-membered heterocycle, wherein the 4- to 6-membered heterocycle is optionally substituted with one or more substituents selected from halogen. In some cases, each R⁸ is independently selected from

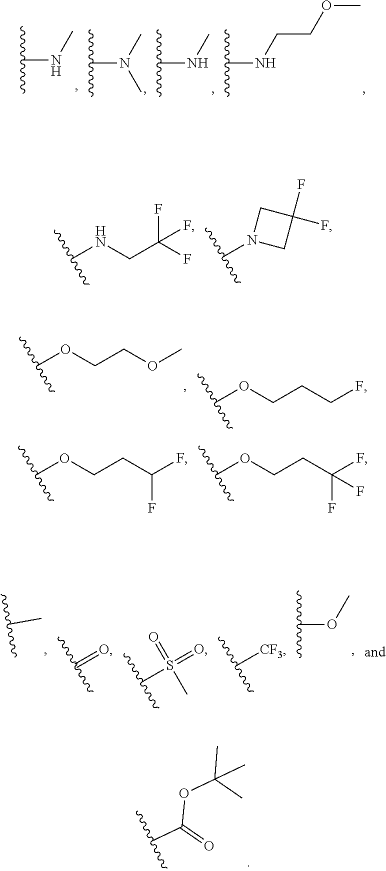

In some cases, R⁷ is selected from

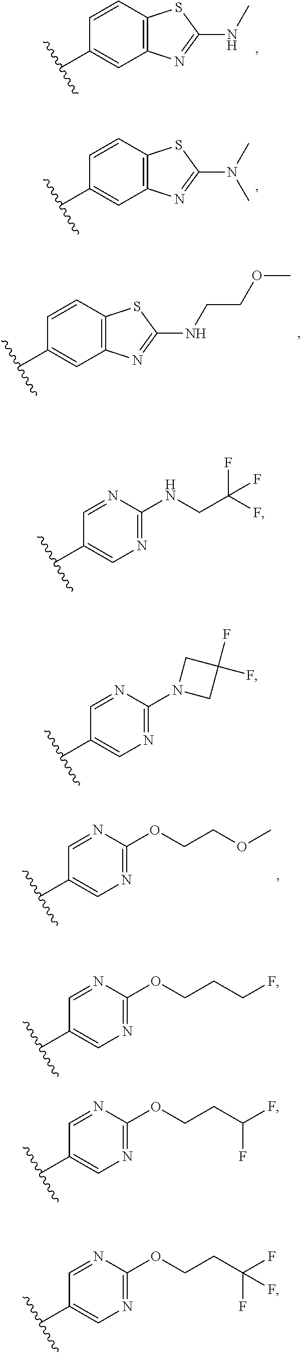

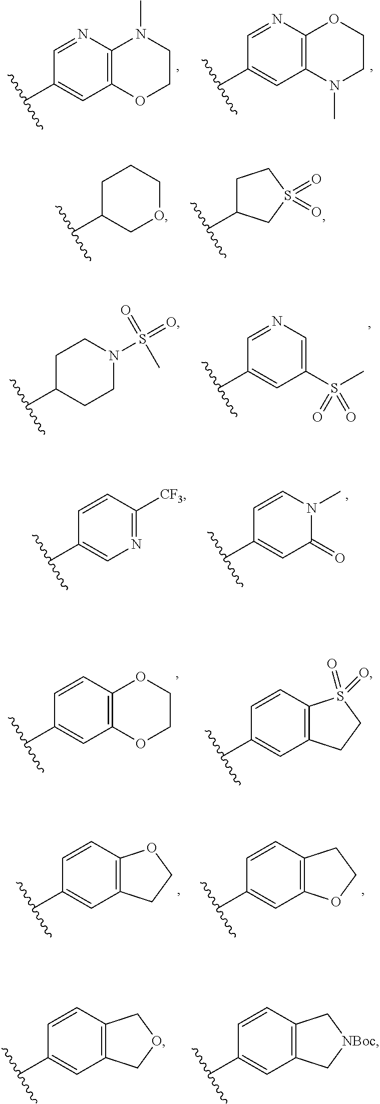

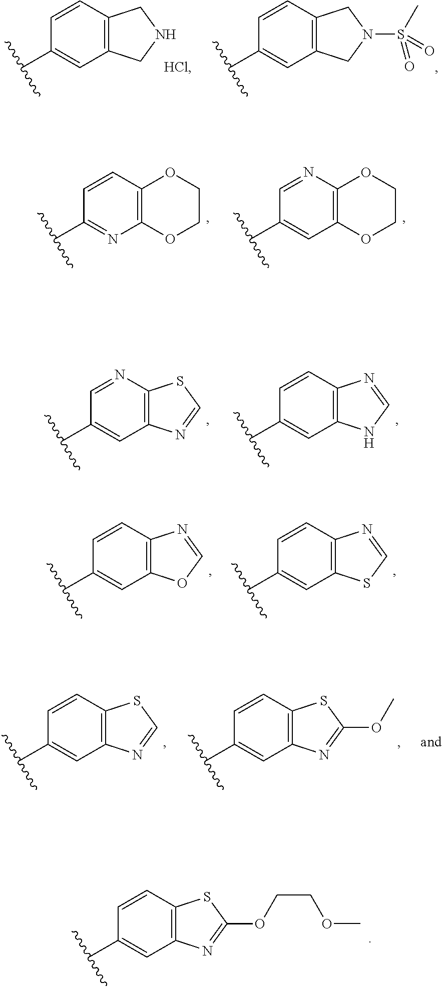

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from a 6-membered heterocycle, each of which is optionally substituted with one or more R⁸. In some cases, the heterocycle has one oxygen atom. In some cases, the heterocycle has one nitrogen atom. In some cases, the heterocycle has one sulfur atom. In some cases, the heterocycle has only one heteroatom. In some cases, the heterocycle has only two heteroatoms. In some cases, the heterocycle has only three heteroatoms. In some cases, R⁷ is selected from an 6-membered heteroaryl, which is optionally substituted with one or more R⁸. In some cases, the heteroaryl has one oxygen atom. In some cases, the heteroaryl has one nitrogen atom. In some cases, the heteroaryl has one sulfur atom. In some cases, the heteroaryl has only one heteroatom. In some cases, the heteroaryl has only two heteroatoms. In some cases, the heteroaryl has only three heteroatoms. In some cases, the heteroaryl has one sulfur atom. In some cases, the 6-membered heteroaryl has one nitrogen atom. In some cases, R⁷ is selected from

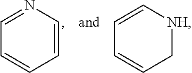 [SMILES: c1ccncc1 ; C1=CCNC=C1]

each of which is optionally substituted with one or more R⁸. In some cases, R⁷ is selected from

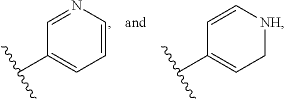 [SMILES: *c1cccnc1 ; *C1=CCNC=C1]

each of which is optionally substituted with one or more R⁸. In some cases, each R⁸ is independently selected from halogen, —OR²⁰, —S(O)₂(R²⁰), —S)₂N(R²⁰)₂, —S(O)N(R²⁰)₂, —S(O)R²⁰(═NR²⁰), —CN, ═O, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, and C₁₋₆ alkyl. In some cases, each R⁸ is independently selected from halogen, —S(O)₂(R²⁰), ═O, C₁₋₆ alkyl, and C₁₋₆ haloalkyl. In some cases, In some cases, R²⁰ is C₁₋₆ alkyl. In some cases, each R⁸ is independently selected from CF₃, Me, ═O, or —S(O)₂(C₁₋₆ alkyl). In some cases, R⁷ is selected from

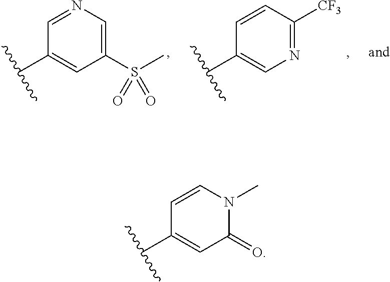

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from 9- to 10-membered heterocycle, each of which are optionally substituted with one or more R⁸. In some cases, R⁷ is selected from 9-membered heterocycle, which optionally substituted with one or more R⁸. In some cases, R⁷ is selected from 10-membered heterocycle, which optionally substituted with one or more R⁸. In some cases, the 9- to 10-membered heterocycle is bicyclic. In some cases, the 9- to 10-membered heterocycle is a fused heterocycle. In some cases, the 9- to 10-membered heterocycle is aromatic. In some cases, the 9- to 10-membered heterocycle is non-aromatic. In some cases, the 9- to 10-membered heterocycle is fully saturated. In some cases, the 9- to 10-membered heterocycle is partially saturated. In some cases, the 9- to 10-membered heterocycle is non-saturated. In some cases, the heterocycle has one oxygen atom. In some cases, the heterocycle has one nitrogen atom. In some cases, the heterocycle has one sulfur atom. In some cases, the heterocycle has only one heteroatom. In some cases, the heterocycle has only two heteroatoms. In some cases, the heterocycle has only three heteroatoms. In some cases, the heterocycle has only four heteroatoms. In some cases, the heterocyle has a combination of different heteroatoms. In some cases, the 9- to 10-membered heterocycle is not fully saturated. In some cases, R⁷ is selected from

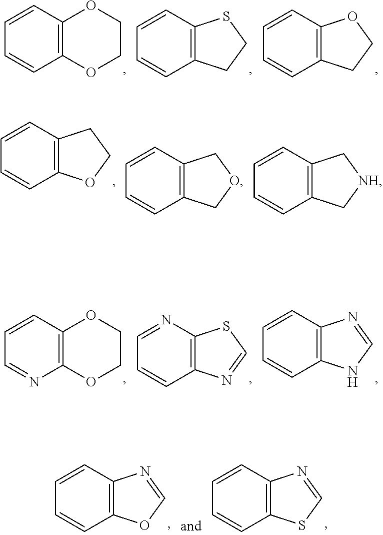

each of which are optionally substituted with one or more R⁸. In some cases, R⁷ is

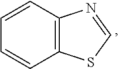

which is optionally substituted with one or more R⁸. In some cases, R⁷ is selected from

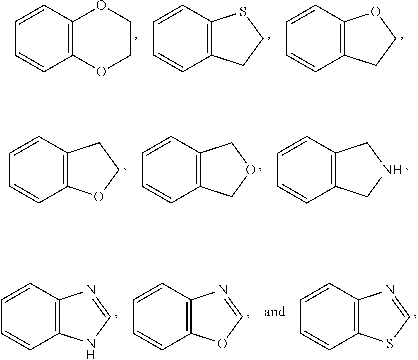

each of which are optionally substituted with one or more R⁸. In some cases, R⁷ is selected from

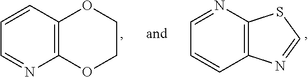 [SMILES: c1cnc2c(c1)OCCO2 ; c1cnc2scnc2c1]

each of which are optionally substituted with one or more R⁸. In some cases, R⁷ is selected from

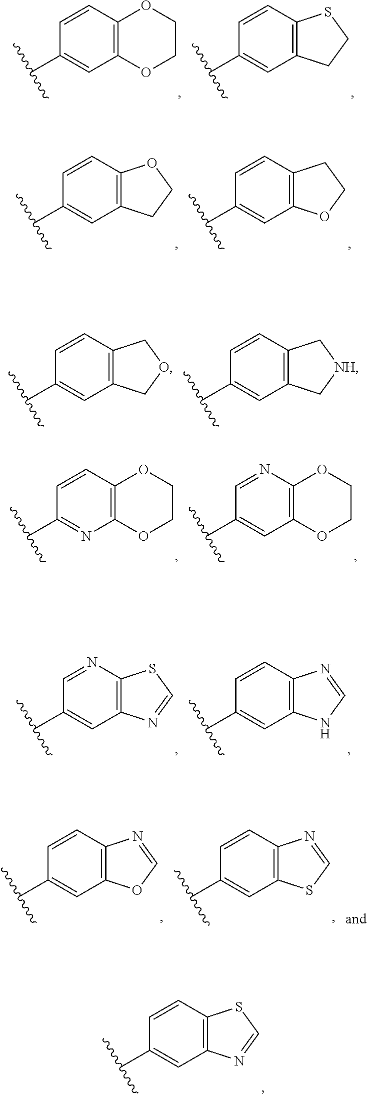

each of which are optionally substituted with one or more R⁸. In some cases, R⁷ is selected from

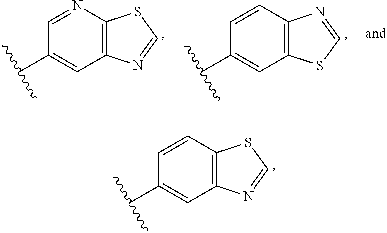 [SMILES: *c1cnc2scnc2c1 ; *c1ccc2ncsc2c1 ; *c1ccc2scnc2c1]

each of which are optionally substituted with one or more R⁸. In some cases, R⁷ is selected from,

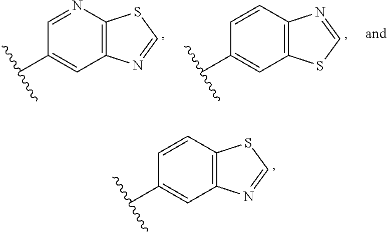 [SMILES: *c1cnc2scnc2c1 ; *c1ccc2ncsc2c1 ; *c1ccc2scnc2c1]

each of which are optionally substituted with one or more R⁸. In some cases, R⁷ is substituted with one to six R⁸. In some cases, R⁷ is substituted with one to three R⁸. In some cases, each R⁸ is independently selected from halogen, —OR²⁰, —S(O)₂(R²⁰), —C(O)OR²⁰, ═O, —N(R²⁰)₂, C₁₋₆ alkyl, and C₁₋₆ haloalkyl. In some cases, each R⁸ is independently selected from halogen, —OR²⁰, —S(O)₂(R²⁰), —N(R²⁰)₂, —C(O)OR²⁰, ═O, C₁₋₆ alkyl, and C₁₋₆ haloalkyl. In some cases, each R⁸ is independently selected from ═O, —S(O)₂(C₁₋₆ alkyl), —CF₃, —NHCH₃, —N(CH₃)₂, —CH₃, —C(O)OC₁₋₆ alkyl, —OMe,

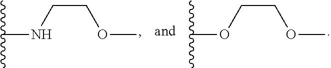

In some cases, each R²⁰ is C₁₋₆ alkyl, wherein each is optionally substituted with one, two, three substituents independently selected from halogen, —OH, —O—C₁₋₆ alkyl. In some cases, each R²⁰ is methyl, ethyl, tert-butyl, and

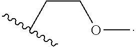 [SMILES: *CCOC]

In some cases, R⁷ is selected from

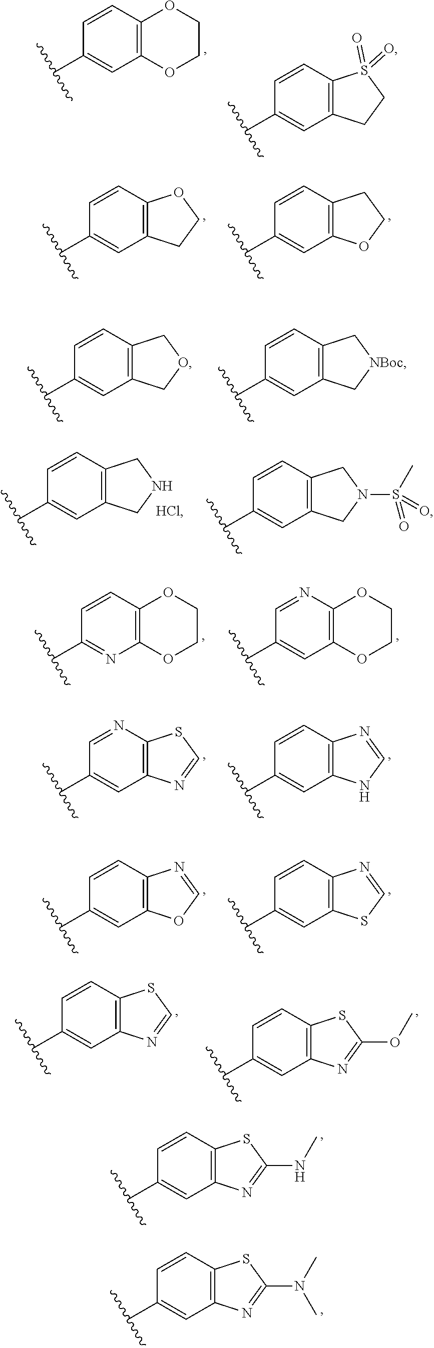

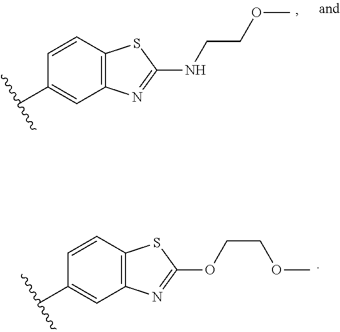 [SMILES: *c1ccc2sc(NCCOC)nc2c1 ; *c1ccc2sc(OCCOC)nc2c1]

In some cases, R⁷ is selected from

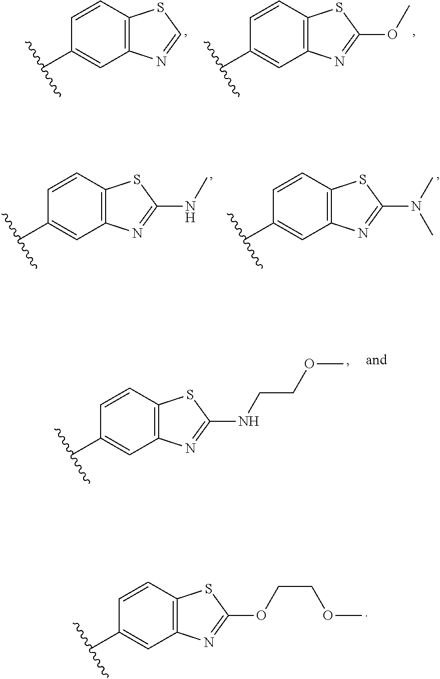 [SMILES: *c1ccc2scnc2c1 ; *c1ccc2sc(OC)nc2c1 ; *c1ccc2sc(NC)nc2c1 ; *c1ccc2sc(N(C)C)nc2c1 ; *c1ccc2sc(NCCOC)nc2c1 ; *c1ccc2sc(OCCOC)nc2c1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁸ is selected from —S(O)₂(NR₂²⁰) and —S(O)₂(R²⁰). In some cases, R⁸ is selected from —S(O)₂(NR₂²⁰). In some cases, R²⁰ is independently selected at each occurrence from hydrogen; C₁₋₆ alkyl, and 3- to 6-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from hydrogen and C₁₋₆ alkyl. In some cases, R²⁰ is independently selected at each occurrence from C₁₋₆ alkyl. In some cases, R⁸ is selected from

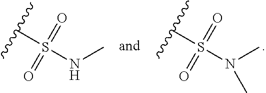

In some cases, R⁷ is

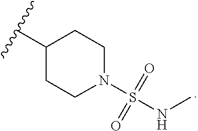 [SMILES: *C1CCN(S(=O)(=O)NC)CC1]

In some cases, R⁷ is

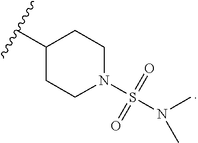 [SMILES: *C1CCN(S(=O)(=O)N(C)C)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁸ is selected from —S(O)₂(R²⁰). In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is 3- to 6-membered heterocycle. In some cases, R²⁰ is 6-membered heterocycle. In some cases, R²⁰ is saturated 6-membered heterocycle. In some cases, the heterocycle contains at least one oxygen atom. In some cases, the heterocycle contains at least one nitrogen atom. In some cases, the heterocycle contains at least one oxygen atom and at least one nitrogen atom. In some cases, R⁸ is

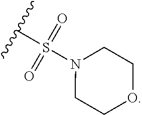 [SMILES: *S(=O)(=O)N1CCOCC1]

In some cases, R⁷ is selected from

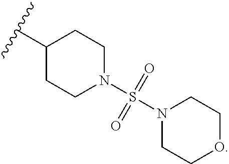 [SMILES: *C1CCN(S(=O)(=O)N2CCOCC2)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁸ is selected from —C(O)R²⁰. In some cases, R²⁰ is independently selected at each occurrence from C₁₋₆ alkyl which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl which is optionally substituted with one or more substituents independently selected from —O—C₁₋₁₀ alkyl, and 3- to 12-membered heterocycle. In some cases, R²⁰ is independently selected at each occurrence from C₁₋₆ alkyl which is optionally substituted with one or more substituents independently selected from halogen, —OH, —CN, —NO₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl which is optionally substituted with 3- to 6-membered heterocycle. In some cases, R²⁰ is independently selected at each occurrence from C₁₋₆ alkyl which is optionally substituted with 6-membered heterocycle. In some cases, the heterocycle contains at least one oxygen atom. In some cases, the heterocycle contains at least one nitrogen atom. In some cases, the heterocycle contains at least one oxygen atom and at least one nitrogen atom. In some cases, the 6-membered heterocycle is selected from morpholine. In some cases, R⁸ is

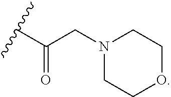 [SMILES: *C(=O)CN1CCOCC1]

In some cases, R⁸ is selected from

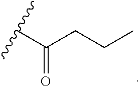 [SMILES: *C(=O)CCC]

In some cases, R⁸ is selected from

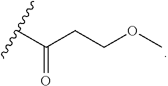 [SMILES: *C(=O)CCOC]

In some cases, R⁷ is selected from

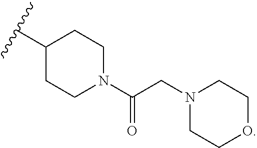 [SMILES: *C1CCN(C(=O)CN2CCOCC2)CC1]

In some cases, R⁷ is selected from

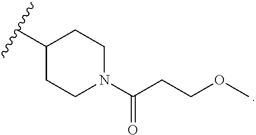 [SMILES: *C1CCN(C(=O)CCOC)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is represented by

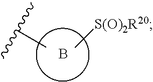

wherein B is selected from an unsubstituted 5- to 6-membered heterocycle. In some cases, R⁷ is represented by

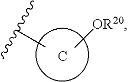

wherein C is selected from a C₅₋₆ saturated carbocycle and R²⁰ is selected from C₁₋₆ alkyl substituted by one-O—C₁₋₁₀ alkyl. In some cases, R⁷ is represented by

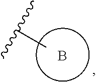

wherein B is selected from an unsubstituted 5- to 6-membered heterocycle. In some cases, R⁷ is represented by

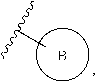

wherein B is selected from a 4- to 6-membered heterocycle having at least one sulfur atom, wherein the 4- to 6-membered heterocycle is substituted with two ═O. In some cases, R⁷ is represented by

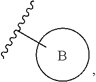

wherein B is selected from an unsubstituted 4- to 6-membered heterocycle having at least one oxygen atom.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁸ is selected from —S(O)₂(R²⁰). In some cases, R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is independently selected at each occurrence from C₁₋₆ alkyl, which is optionally substituted with one or more substituents independently selected from halogen, —OH, —CN, —NO₂, —NH₂, —C₁₋₁₀ haloalkyl, and —O—C₁₋₁₀ alkyl. In some cases, R²⁰ is independently selected at each occurrence from C₁₋₆ alkyl, which is optionally substituted with one or more-O—C₁₋₁₀ alkyl. In some cases, R²⁰ is independently selected at each occurrence from C₁₋₆ alkyl. In some cases, R²⁰ is independently selected at each occurrence from methyl, ethyl and propyl. In some cases, R²⁰ is independently selected at each occurrence from C₃₋₆ carbocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is independently selected at each occurrence from C₃₋₆ carbocycle, which is optionally substituted with one or more substituents independently selected from halogen, C₁₋₁₀ alkyl, —C₁₋₁₀ haloalkyl, and —O—C₁₋₁₀ alkyl. In some cases, R²⁰ is independently selected at each occurrence from C₃₋₆ carbocycle. In some cases, R⁸ is selected from,

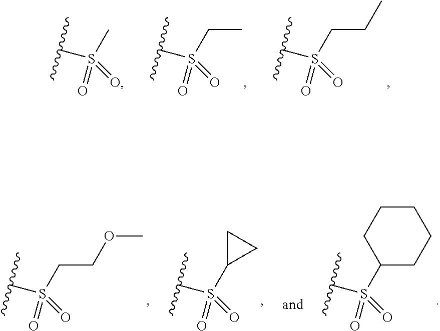

In some cases, R⁸ is selected from,

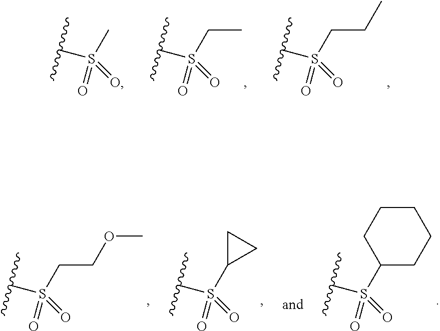

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from substituted C₆ cycloalkyl and optionally substituted saturated 6-membered heterocycle. In some cases, the heterocycle has at least one nitrogen atom. In some cases, the heterocycle has at least one oxygen atom. In some cases, the heterocycle is unsubstituted. In some cases, the heterocycle is substituted. In some cases, the heterocycle is selected from tetrahydropyran and piperidine. In some cases, each R⁸ is independently selected from halogen, —C(O)N(R²⁰)₂, —S(O)₂(R²⁰), and —S(O)₂(NR₂²⁰). In some cases, R⁷ is selected from

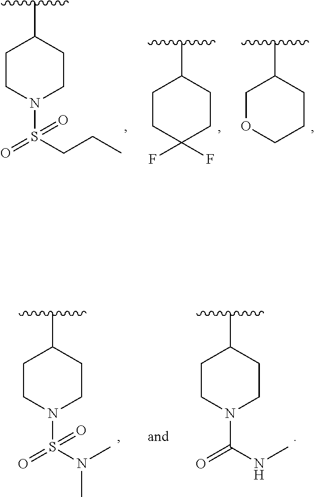

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸ is independently selected from —C(O)R²⁰, —C(O)N(R²⁰)₂, —S(O)₂(R²⁰), and —S(O)(NR²⁰) R²⁰. In some cases, each R⁸ is independently selected from —C(O)N(R²⁰)₂ and —S(O)₂(R²⁰), and —S(O)(NR²⁰) R²⁰. In some cases, each R⁸ is independently selected from —S(O)₂(R²⁰), and —S(O)(NR²⁰) R²⁰. In some cases, each R⁸ is-C(O)N(R²⁰)₂. In some cases, each R⁸ is —S(O)(NR²⁰) R²⁰. In some cases, R²⁰ is independently selected at each occurrence from hydrogen: C₁₋₆ alkyl, and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl, which is optionally substituted with one or more substituents independently selected from halogen, —OH, —CN, —NO₂, —NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl, which is optionally substituted with one or more substituents independently selected from halogen, —OH, —CN, —NO₂, —NH₂, —O—C₁₋₁₀ alkyl. In some cases, R²⁰ is independently selected at each occurrence from C₁₋₆ alkyl. In some cases, R²⁰ is methyl. In some cases, R⁸ is selected from,

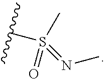 [SMILES: *S(C)(=O)=NC]

In some cases, R⁸ is selected from,

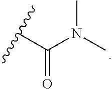 [SMILES: *C(=O)N(C)C]

In some cases, R⁸ is selected from,

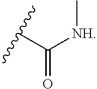 [SMILES: *C(=O)NC]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₆ cycloalkyl optionally substituted with one or more R⁸. In some cases, R⁸ is independently selected at each occurrence from halogen, C₁₋₆ haloalkyl, —C(O)R²⁰, —C(O)OR²⁰, —C(O)N(R²⁰)₂, —N(R²⁰)C(O)R²⁰ —N(R²⁰)C(O)N(R²⁰)₂, —S(O)₂(R²⁰), —S(O)(R²⁰), and —S(O)₂(NR₂²⁰). In some cases, R⁸ is selected at each occurrence from halogen, C₁₋₆ haloalkyl, —N(R²⁰)C(O)R²⁰, —N(R²⁰)C(O)N(R²⁰)₂, —S(O)₂(R²⁰), —S(O)(R²⁰), and —S(O)₂(NR₂²⁰). In some cases, R⁸ is selected at each occurrence from halogen, C₁₋₆ haloalkyl, —N(R²⁰)C(O)R²⁰, and —N(R²⁰)C(O)N(R²⁰)₂. In some cases, R⁸ is selected at each occurrence from halogen and C₁₋₆ haloalkyl. In some cases, R⁸ is selected at each occurrence from —N(R²⁰)C(O)R²⁰ and —N(R²⁰)C(O)N(R²⁰)₂. In some cases, R⁸ is selected at each occurrence from halogen. In some cases, R⁸ is independently selected at each occurrence from halogen, C₁₋₆ haloalkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)(NR²⁰)R²⁰, and —S(O)(NR²⁰)N(R²⁰)₂. In some cases, R⁸ is independently selected at each occurrence from halogen, C₁₋₆ haloalkyl, —C(O)R²⁰, —S(O)₂(R²⁰), —S(O)(R²⁰), —S(O)(NR²⁰)R²⁰, and —S(O)(NR²⁰)N(R²⁰)₂. In some cases, R⁸ is independently selected at each occurrence from halogen, —C(O)R²⁰, and —S(O)₂(R²⁰). In some cases, R²⁰ is independently selected at each occurrence from hydrogen, and C₁₋₆ alkyl. In some cases, R²⁰ is independently selected at each occurrence from hydrogen, and C₁₋₆ alkyl. In some cases, R⁸ is independently selected each occurrence from —OR²⁰, —SR²⁰, —N(R²⁰)₂, —NO₂, ═O, —S, ═N(R²⁰), —CN, C₁₋₆ aminoalkyl, C₁₋₆ hydroxyalkyl, C₁₋₆ cyanoalkyl, C₁₋₆ haloalkyl, and C₁₋₆ alkyl. In some cases, R⁸ is selected each occurrence from —OR²⁰, —SR²⁰, —N(R²⁰)₂, —NO₂, ═O, —S, ═N(R²⁰), and —CN. In some cases, R⁸ is selected each occurrence from —OR²⁰, —SR²⁰, and —N(R²⁰)₂. In some cases, R⁸ is selected each occurrence from —OR²⁰. In some cases, R²⁰ is independently selected at each occurrence from hydrogen, and C₁₋₆ alkyl. In some cases, R²⁰ is independently selected at each occurrence from hydrogen, and C₁₋₆ alkyl.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₆ cycloalkyl substituted with one or more R⁸. In some cases, R⁷ is

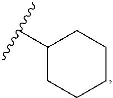 [SMILES: *C1CCCCC1]

which is substituted with one or more R⁸. In some cases, R⁷ is

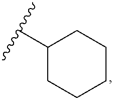 [SMILES: *C1CCCCC1]

which is substituted with two R⁸. In some cases, R⁸ is independently selected at each occurrence from halogen, and C₁₋₆ haloalkyl. In some cases, R⁷ is selected from

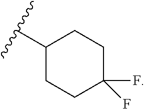 [SMILES: *C1CCC(F)(F)CC1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₈₋₁₀ cycloalkyl, each of which is optionally substituted with one or more R⁸. In some cases, R⁷ is selected from

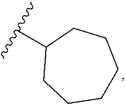 [SMILES: *C1CCCCCC1]

which is optionally substituted with one or more R⁸. In some cases, R⁷ is selected from C₈ cycloalkyl, which is optionally substituted with one or more R⁸. In some cases, the C₈ cycloalkyl is selected from

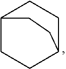

which is optionally substituted with one or more R⁸. In some cases, R⁷ is

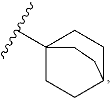 [SMILES: *C12CCC(CC1)CC2]

which is optionally substituted with one or more R⁸. In some cases, R⁷ is

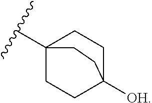 [SMILES: *C12CCC(O)(CC1)CC2]

In some cases, R⁷ is selected from C₁₀ cycloalkyl, which is optionally substituted with one or more R⁸. In some cases, R⁷ is

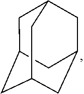

which is optionally substituted with one or more R⁸. In some cases, R⁷ is selected from

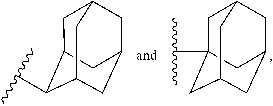 [SMILES: *C1C2CC3CC(C2)CC1C3 ; *C12CC3CC(CC(C3)C1)C2]

which is optionally substituted with one or more R⁸. In some cases, each R⁸ is independently selected at each occurrence from halogen, —OR²⁰, —N(R²⁰)₂, —O, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)₂(NR₂²⁰), —S(O)(NR²⁰)R²⁰, —S(O)(NR²⁰)N(R²⁰)₂, and 4- to 8-membered heterocycle. In some cases, each R⁸ is independently selected at each occurrence from —OR²⁰, C₁₋₆ aminoalkyl, C₁₋₆ hydroxyalkyl, C₁₋₆ cyanoalkyl, C₁₋₆ haloalkyl, C₁₋₆ alkoxyalkyl, and C₁₋₆ alkyl. In some cases, each R⁸ is independently selected at each occurrence from —OR²⁰, and C₁₋₆ alkyl. In some cases, each R⁸ is independently selected at each occurrence from —OH, and —O—C₁₋₆ alkyl. In some cases, R⁷ is

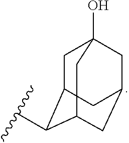 [SMILES: *C1C2CC3CC1CC(O)(C3)C2]

In some cases, R⁷ is

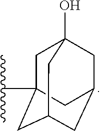 [SMILES: *C12CC3CC(C1)CC(O)(C3)C2]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₃₋₅ cycloalkyl, C₇₋₈ cycloalkyl, and C₉₋₁₀ cycloalkyl, each of which is optionally substituted with one or more R⁸. In some cases, R⁷ is selected from C₃ cycloalkyl, C₅ cycloalkyl, C₇₋₈ cycloalkyl, and C₉₋₁₀ cycloalkyl, each of which is optionally substituted with one or more R⁸. In some cases, R⁷ is selected from

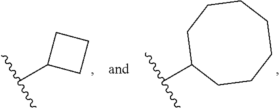 [SMILES: *C1CCC1 ; *C1CCCCCCC1]

each of which is optionally substituted with one or more R⁸. In some cases, R⁸ is independently selected at each occurrence from each halogen, —OR²⁰, —SR²⁰, —N(R²⁰)₂, —NO₂, ═O, —S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)₂(NR₂²⁰), —S(O)(N R²⁰)R²⁰, and —S(O)(NR²⁰)N(R²⁰)₂. In some cases, R⁸ is independently selected at each occurrence from each halogen, —SR²⁰, —N(R²⁰)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²⁰)₂, —N(R²⁰)C(O)R²⁰, —N(R²⁰)C(O)N(R²⁰)₂, —S(O)₂(R²⁰), —S(O)(R²⁰), —S(O)₂(NR₂²⁰), —S(O)(NR²⁰)R²⁰, and —S(O)(N R²⁰)N(R²⁰)₂. In some cases, R⁸ is selected from —N(R²⁰)C(O)R²⁰, —N(R²⁰)C(O)N(R²⁰)₂, —S(O)₂(R²⁰), —S(O)(R²⁰), —S(O)₂(NR₂²⁰), —S(O)(N R²⁰)R²⁰, and —S(O)(NR²⁰)N(R²⁰)₂. In some cases, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, and —C₁₋₁₀ haloalkyl. In some cases, when R¹ is N, R³ is imidazole, R⁷ is C₄ cycloalkyl, and R⁸ is —OR²⁰, R²⁰ is selected from C₁ alkyl, C₃₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, when R¹ is N, R³ is imidazole, R⁷ is C₄ cycloalkyl, and R⁸ is —OR²⁰, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₂₋₁₀ alkyl, C₂₋₁₀ alkenyl, C₂₋₁₀ alkynyl, C₃₋₁₂ carbocycle, and 3- to 12-membered heterocycle. In some cases, R⁷ is

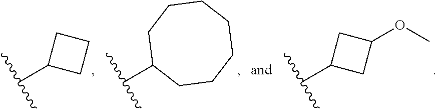

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted C₅ cycloalkyl. In some cases, the C₅ cycloalkyl is substituted. In some cases, R⁷ is C₅ cycloalkyl substituted with one or more fluorine atoms. In some cases, R⁷ is selected

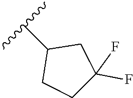 [SMILES: *C1CCC(F)(F)C1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸ is independently selected at each occurrence from halogen, —OR²⁰, —SR²⁰, —N(R²⁰)₂, —NO₂, —O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²⁰)₂, —N(R²⁰)C(O)R²⁰—N(R²⁰)C(O)N(R²⁰)₂, —S(O)₂(R²⁰), —S(O)(R²⁰), —S(O)₂(NR₂²⁰), —S(O)(NR²⁰)R²⁰, and —S(O)(NR²⁰)N(R²⁰)₂.

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted 5- to 6-membered heteroaryl. In some cases, R⁷ is selected from an optionally substituted 6-membered heteroaryl. In some cases, R⁷ is selected from an optionally substituted pyridine. In some cases, each R⁸ is selected from halogen, —N(R²⁰)₂, OR²⁰, —S(O)₂(R²⁰), —S)₂N(R²⁰)₂, —S(O)N(R²⁰)₂, —S(O)R²⁰(═NR²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, and C₁₋₆ alkyl. In some cases, each R⁸ is selected from halogen, —OR²⁰, —CN, C₁₋₆ hydroxyalkyl, C₁₋₆ cyanoalkyl, C₁₋₆ haloalkyl, and C₁₋₆ alkyl. In some cases, R⁷ is selected from

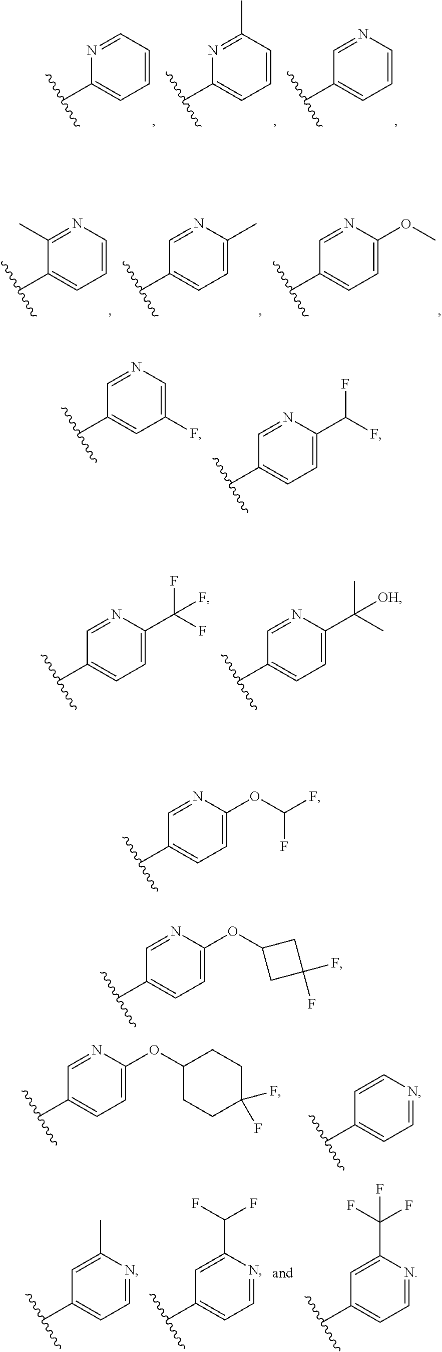

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted phenyl. In some cases, each R⁸ is selected halogen, —N(R²⁰)₂, —OR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl. In some cases, each R⁸ is selected halogen, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl. In some cases, each R²⁰ is selected form hydrogen and C₁₋₆ alkyl. In some cases, R⁷ is selected from phenyl optionally substituted with one or more substituents selected from halogen and C₁₋₆ hydroxyalkyl. In some cases, R⁷ is selected from

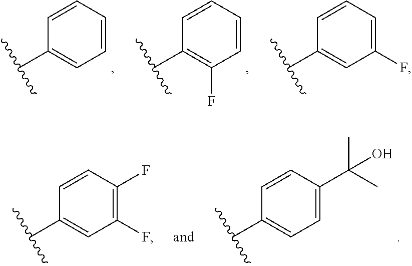 [SMILES: *c1ccccc1 ; *c1ccccc1F ; *c1cccc(F)c1 ; *c1ccc(F)c(F)c1 ; *c1ccc(C(C)(C)O)cc1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from an optionally substituted phenyl. In some cases, R⁷ is

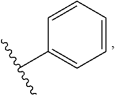 [SMILES: *c1ccccc1]

which is optionally substituted with one or more R⁸. In some cases, each R⁸ is independently selected from halogen, and —S(O)₂(R²⁰). In some cases, each R⁸ is independently selected from halogen and —S(O)₂(CH₃). In some cases, R⁷ is selected

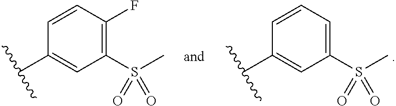 [SMILES: *c1ccc(F)c(S(C)(=O)=O)c1 ; *c1cccc(S(C)(=O)=O)c1]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₈-C₁₂ carbocycle, which is optionally substituted with one or more R⁸. In some cases, the carbocycle is bicyclic. In some cases, the carboyclce is non-aromatic. In some cases, R⁷ is selected from C₉ carbocycle, which is optionally substituted with one or more R⁸. In some cases, R⁷ is

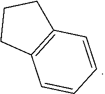

In some cases, R⁷ is

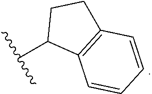 [SMILES: *C1CCc2ccccc21]

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from C₅-C₁₀ carbocycle and 5- to 10-membered heterocycle, each of which are optionally substituted with one or more R⁸. In some cases, the carbocycle is bicyclic. In some cases, the carbocycle is monocyclic. In some cases, the heterocycle is monocyclic. In some cases, R⁷ is selected from

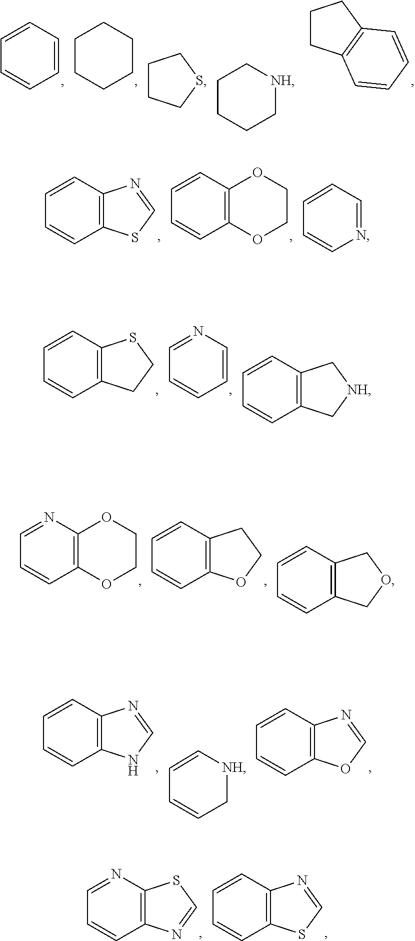

each of which is optionally substituted with one or more R⁸. In some cases, R⁷ is selected from

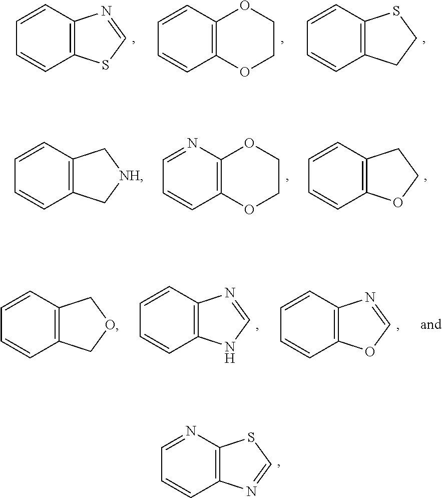

each of which is optionally substituted with one or more R⁸. In some cases, R⁷ is selected from

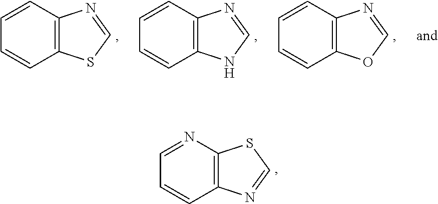

each of which is optionally substituted with one or more R⁸. In some cases, R⁷ is selected from

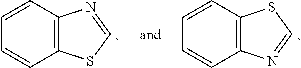 [SMILES: c1ccc2scnc2c1 ; c1ccc2scnc2c1]

each of which is optionally substituted with one or more R⁸. In some cases, R⁷ is

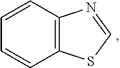

each of which is optionally substituted with one or more R⁸. In some cases, the heterocycle is bicyclic. In some cases, each R⁸ is independently selected from halogen, —N(R²⁰)₂, —OR²⁰, —S(O)₂(R²⁰), —NR²⁰S(O)₂R²⁰, —C(O)N(R²⁰)₂, C₁₋₆ haloalkyl, C₁₋₆ alkyl and ═O. In some cases, each R⁸ is independently selected from fluorine, —S(O)₂(C₁₋₆ alkyl),

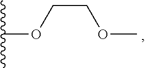 [SMILES: *OCCOC]

—C(O)NHCH₃, —NHS(O)₂C₁₋₆ alkyl, —NHC₁₋₆ alkyl, —NHS(O)₂C₁₋₆ alkyl-O—C₁₋₆ alkyl, ═O, —OMe, —CF₃, and —CH₃. In some cases, R⁷ is selected from

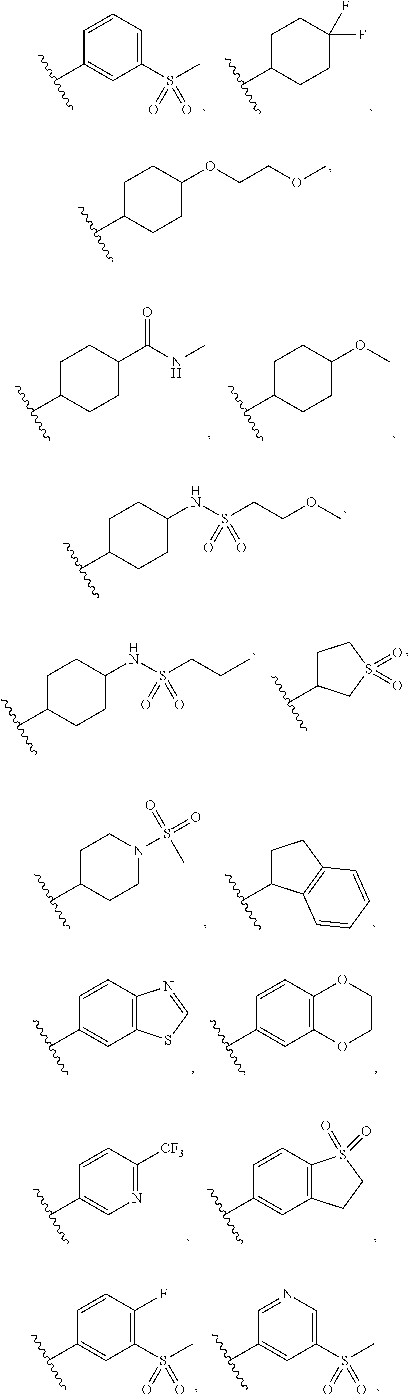

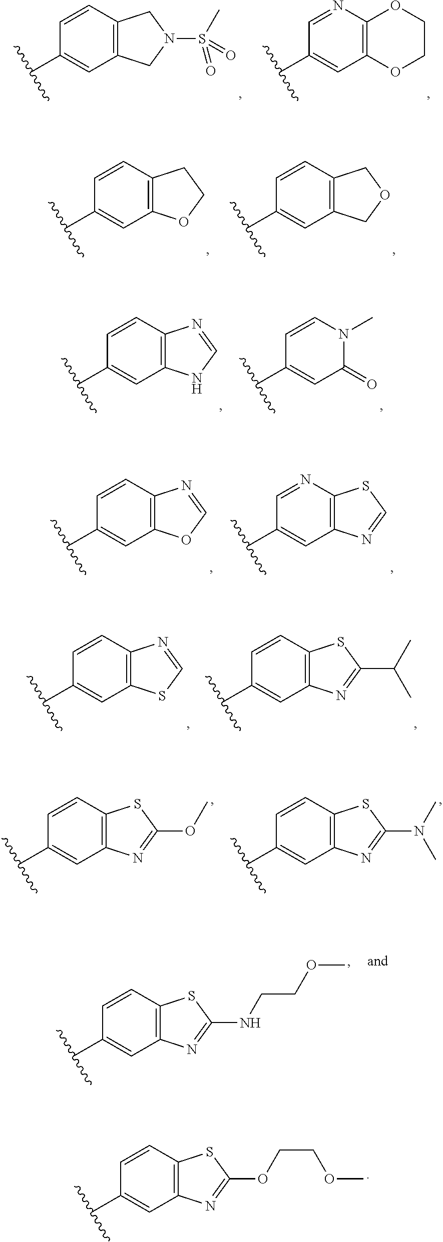

In some cases, R⁷ is selected from

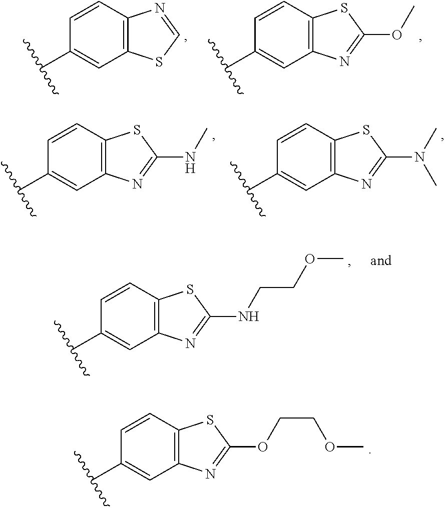 [SMILES: *c1ccc2ncsc2c1 ; *c1ccc2sc(OC)nc2c1 ; *c1ccc2sc(NC)nc2c1 ; *c1ccc2sc(N(C)C)nc2c1 ; *c1ccc2sc(NCCOC)nc2c1 ; *c1ccc2sc(OCCOC)nc2c1]

In an aspect, the present disclosure provides a compound represented by the structure of Formula (II):

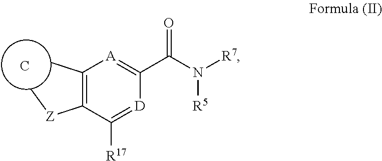

or a pharmaceutically acceptable salt or solvate thereof; wherein

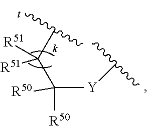

Z is selected from, wherein t represents the point of connection between Z and \textcircled{C};
Y is selected from —O—, —NR⁹—, —S—, and —SO₂—;
each R⁵⁰ is independently selected from hydrogen, halogen, C₁-C₆ alkyl; or come together to form

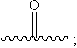 [SMILES: *=O]

each R⁵¹ is independently selected from hydrogen, halogen, and C₁-C₆ alkyl;
k is selected from 1 and 2;
A is selected from N and CR¹⁸;
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen; and C₁₋₁₀ alkyl, C₂₋₁₀ alkenyl, C₂₋₁₀ alkynyl, C₃-C₁₄ carbocycle and 5- to 15-membered heterocycle, each of which are optionally substituted with one or more R⁸.
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, OXO, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
each R⁸* is independently selected from 4- to 12-membered heterocycle, wherein the 4- to 12-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, OXO, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰ —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, —S(O)₂(C₁₋₆ alkyl), C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (II):
Z is selected from

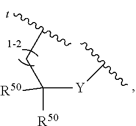

wherein t represents the point of connection between Z and \textcircled{C};
Y is selected from —O—, —NR⁹—, —S—, and —SO₂—;
each R⁵⁰ is independently selected from hydrogen, halogen, C₁-C₆ alkyl; or come together to form

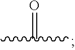 [SMILES: *=O]

A is selected from N and CR¹⁸.
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen, C₁₋₁₀ alkyl, C₂₋₁₀ alkenyl, C₂₋₁₀ alkynyl, C₃-C₁₄ carbocycle and 5- to 15-membered heterocycle, each of which are optionally substituted with one or more R⁸.
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 5- to 12-membered heterocycle, wherein the C₃-C₁₂ carbocycle and 5- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, C₂₋₁₀ alkynyl;
R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen: C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (II), \textcircled{C} is selected from an optionally substituted imidazole, wherein the imidazole is optionally substituted with one or more R¹⁴. In some cases, \textcircled{C} is selected from

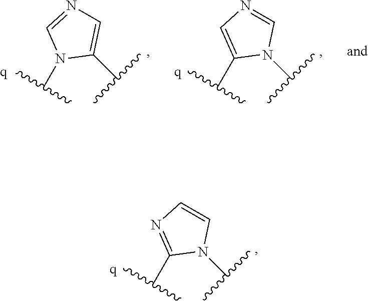

wherein q represents the point of connection to Z, and wherein each is optionally substituted with one or more R¹⁴. In some cases, \textcircled{C} is selected from

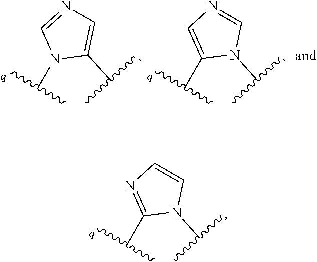 [SMILES: *c1cncn1* ; *c1cncn1* ; *c1nccn1*]

each of which is unsubstituted.

In some embodiments, for a compound or salt of Formula (II), R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₃₋₁₂ carbocycle, and 3- to 12-membered heterocycle. In some cases, R¹⁴ is selected from halogen, C₁₋₁₀ alkyl, —C₁₋₁₀ haloalkyl, —O—C₁₋₁₀ alkyl, and C₃₋₆ carbocycle. In some cases, R¹⁴ is selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₃₋₁₂ carbocycle, and 3- to 12-membered heterocycle. In some cases, R¹⁴ is selected from halogen, C₁₋₁₀ alkyl, —C₁₋₁₀ haloalkyl, —O—C₁₋₁₀ alkyl, and C₃₋₆ carbocycle. In some cases, R¹⁴ is selected from halogen, C₁₋₁₀ alkyl, and C₃₋₆ carbocycle. In some cases, R¹⁴ is selected from halogen and C₁₋₁₀ alkyl.

In some embodiments, for a compound or salt of Formula (II), Y is selected from —O—, —NR⁹—, —S—, and —SO₂. In some cases, Y is —O—. In some cases, Y is —NR⁹—. In some cases, Y is —S—. In some cases, Y is —SO₂. In some cases, Y is selected from —O- and —NR⁹—.

In some embodiments, for a compound or salt of Formula (II), R⁹ is selected from hydrogen, C₁-C₆ alkyl, C₁-C₆ haloalkyl, and C₃₋₆ carbocycle. In some cases, R⁹ is selected from hydrogen and C₁-C₆ alkyl. In some cases, R⁹ is hydrogen. In some cases, R⁹ is C₁-C₆ alkyl.

In some embodiments, for a compound or salt of Formula (II), each R⁵⁰ is independently selected from hydrogen, halogen, and C₁-C₆ alkyl. In some cases, each R⁵⁰ comes together to form

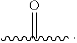 [SMILES: *=O]

In some cases, each R⁵⁰ is hydrogen.

In some embodiments, for a compound or salt of Formula (II), Z is

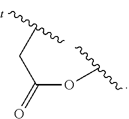 [SMILES: *CC(=O)O*]

In some cases, Z is selected from

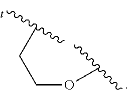 [SMILES: *CCO*]

In some cases, Z is selected from

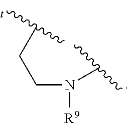

In some cases, Z is selected from

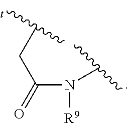

In some cases, Z is selected from

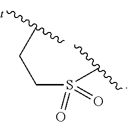 [SMILES: *CCS(*)(=O)=O]

In some cases, Z is selected from

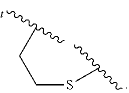 [SMILES: *CCS*]

In some embodiments, for a compound or salt of Formula (II), Z is selected from

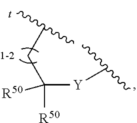

wherein t represents the point of connection between Z and \textcircled{C}. In some cases, Z is selected from

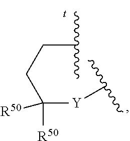

wherein t represents the point of connection between Z and \textcircled{C}. In some cases, Z is selected from

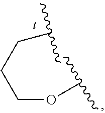

wherein t represents the point of connection between Z and \textcircled{C}.

In some embodiments, for a compound or salt of Formula (II), Z is selected from

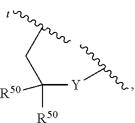

wherein t represents the point of connection between Z and \textcircled{C}.

In some embodiments, for a compound or salt of Formula (II), R⁹ is selected from hydrogen, C₁-C₆ alkyl, and C₃₋₆ carbocycle.

In some embodiments, Formula (II) is represented by Formula (IIA):

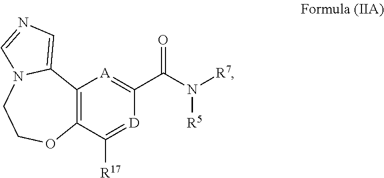

or a pharmaceutically acceptable salt or solvate thereof.

In some embodiments, Formula (II) is represented by Formula (IIB):

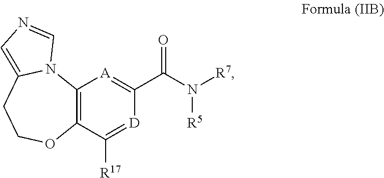

or a pharmaceutically acceptable salt or solvate thereof.

In some embodiments, Formula (II) is represented by Formula (IIC):

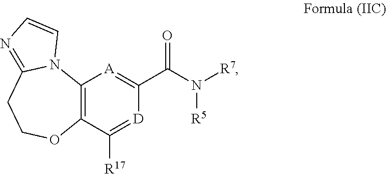

or a pharmaceutically acceptable salt or solvate thereof.

In some embodiments, for a compound or salt of Formula (II), Formula (IIA), Formula (IIB), or Formula (IIC), A is selected from N and CH. In some cases, A is CH. In some cases, A is N.

In some embodiments, for a compound or salt of Formula (II), Formula (IIA), Formula (IIB), or Formula (IIC), A is selected from N, CF, and CH. In some cases, A is selected from N and CF. In some cases, A is selected from CF and CH. In some cases, A is CH. In some cases, A is N. In some cases A is CF.

In some embodiments, for a compound or salt of Formula (II), Formula (IIA), Formula (IIB), or Formula (IIC), D is selected from N and CH. In some cases, D is CH. In some cases, D is N.

In some embodiments, for a compound or salt of Formula (II), Formula (IIA), Formula (IIB), or Formula (IIC), D is selected from N, CF, and CH. In some cases, D is selected from N and CF. In some cases, D is selected from CF and CH. In some cases, D is CH. In some cases, D is N. In some cases, D is CF.

In some embodiments, for a compound or salt of Formula (II), Formula (IIA), Formula (IIB), or Formula (IIC), R¹⁷ is selected from hydrogen, halogen, —N(R²⁰)₂, —OR²⁰, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₆ carbocycle, and 5- to 6-membered heterocycle. In some cases, R¹⁷ is selected from hydrogen, halogen, —N(R²⁰)₂, —OR²⁰, —CN, C₁₋₆ haloalkyl, C₁₋₆ alkyl, C₃₋₆ carbocycle, and 5- to 6-membered heterocycle. In some cases, R¹⁷ is selected from hydrogen, halogen, —O—C₁₋₆ alkyl, C₁₋₆ haloalkyl, C₁₋₆ alkyl, and C₃₋₆ carbocycle. In some cases, R¹⁷ is selected from hydrogen, —Br, —OCH₃, —CF₃, methyl, and cyclopropyl. In some cases, R¹⁷ is hydrogen. In some cases, R¹⁷ is halogen. In some cases, R¹⁷ is C₁₋₆ haloalkyl. In some cases, R¹⁷ is selected from halogen and C₁₋₆ haloalkyl. In some cases, R¹⁷ is —O—C₁₋₆ alkyl. In some cases, R¹⁷ is C₃₋₆ carbocycle. In some cases, R¹⁷ is C₁₋₆ alkyl. In some cases, R¹⁷ is selected from hydrogen and C₁₋₆ alkyl. In some cases, R¹⁷ is selected from hydrogen, C₁₋₆ alkyl, and saturated C₃₋₆ carbocycle.

In some embodiments, Formula (II) or Formula (IIA) is represented by Formula (IID):

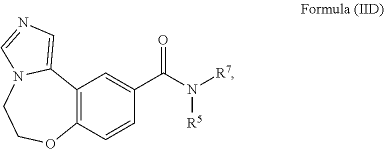

or a pharmaceutically acceptable salt or solvate thereof.

In some embodiments, Formula (II) or Formula (IIB) is represented by Formula (IIE):

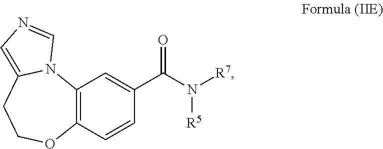

or a pharmaceutically acceptable salt or solvate thereof.

In some embodiments, Formula (II) or Formula (IIC) is represented by Formula (IIF):

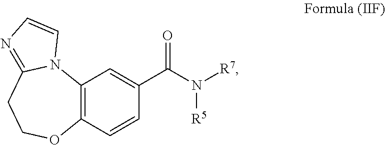

or a pharmaceutically acceptable salt or solvate thereof.

In some embodiments, Formula (II) is represented by Formula (IIF):

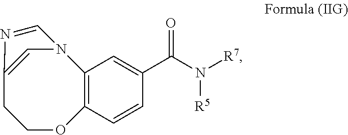

or a pharmaceutically acceptable salt or solvate thereof.

In some embodiments, for a compound or salt of Formula (II), Formula (IID), Formula (IIE), Formula (IIF) or Formula (IIG), R¹⁷ is selected from hydrogen, halogen, —OR²⁰, C₁₋₆ alkyl, C₁₋₆ haloalkyl, saturated C₃₋₆ carbocycle. In some cases, R¹⁷ is selected from hydrogen, F, Cl, Br, —OCH₃, —CF₃, methyl, and cyclopropyl. In some cases, R¹⁷ is hydrogen. In some cases, R¹⁷ is halogen. In some cases, R¹⁷ is C₁₋₆ haloalkyl. In some cases, R¹⁷ is selected from halogen and C₁₋₆ haloalkyl. In some cases, R¹⁷ is —O—C₁₋₆ alkyl. In some cases, R¹⁷ is saturated C₃₋₆ carbocycle. In some cases, R¹⁷ is C₁₋₆ alkyl. In some cases, R¹⁷ is selected from hydrogen and C₁₋₆ alkyl. In some cases, R¹⁷ is selected from hydrogen, C₁₋₆ alkyl, and saturated C₃₋₆ carbocycle.

In some embodiments, for a compound or salt of Formula (II), Formula (IIA), Formula (IIB), Formula (IIC), Formula (IID), Formula (IIE), Formula (IIF), or Formula (IIG), each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —C(O)N(R²⁰)₂, and 4- to 6-membered heterocycle, wherein the 4- to 6-membered heterocycle is optionally substituted with one or more substituents selected from halogen, —OH, —CN, —NH₂, oxo, C₁₋₆ alkyl, —C₁₋₆ haloalkyl, and —O—C₁₋₆ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R²⁰ is independently selected at each occurrence from hydrogen: C₁₋₆ alkyl, C₃₋₆ carbocycle, and 4- to 5-membered heterocycle, each of which is optionally substituted with one or more substituents independently selected from halogen, —OH, —CN, —NH₂, —S(O)₂(C₁₋₆ alkyl), C₁₋₆ alkyl, —C₁₋₆ haloalkyl, —O—C₁₋₆ alkyl, C₃₋₆ carbocycle, and 4- to 5-membered heterocycle. In some cases, each R⁸ is independently selected from F, methyl, ethyl,

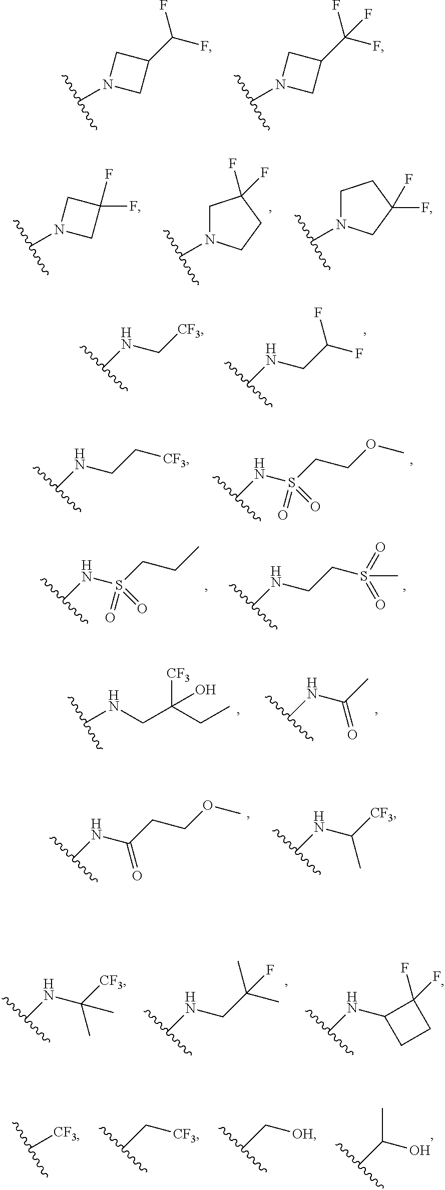

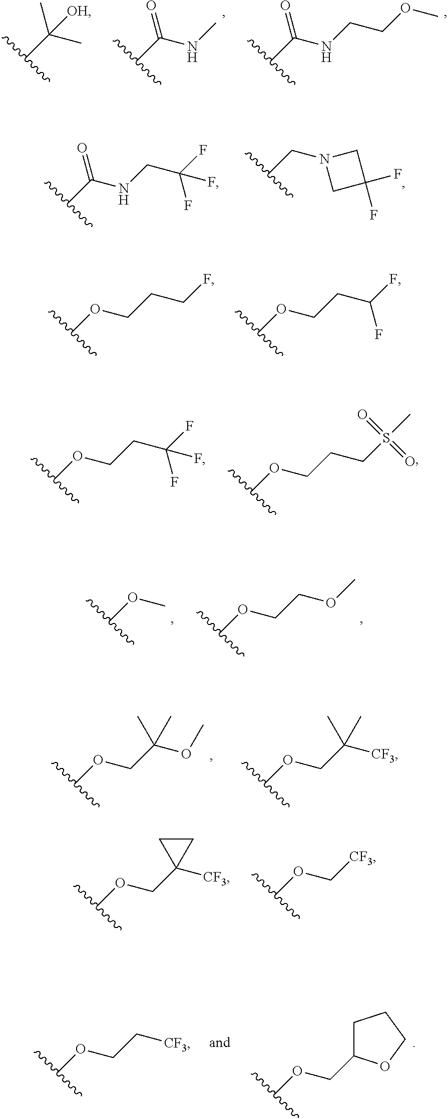

In some embodiments, for a compound or salt of Formula (II), Formula (IIA), Formula (IIB), Formula (IIC), Formula (IID), Formula (IIE), Formula (IIF), or Formula (IIG), R⁷ is selected from C₄-C₆ carbocycle, which is optionally substituted with one or more R⁸. In some cases, R⁷ is selected from

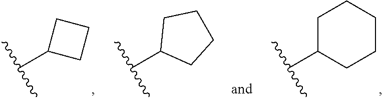 [SMILES: *C1CCC1 ; *C1CCCC1 ; *C1CCCCC1]

which is optionally substituted with one or more R⁸. In some cases, R⁷ is

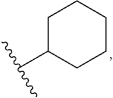 [SMILES: *C1CCCCC1]

which is optionally substituted with one or more R⁸. In some cases, R⁷ is

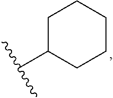 [SMILES: *C1CCCCC1]

which is substituted with one R⁸. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —C(O)N(R²⁰)₂, —S(O)₂(R²⁰), and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from F, methyl, ethyl,

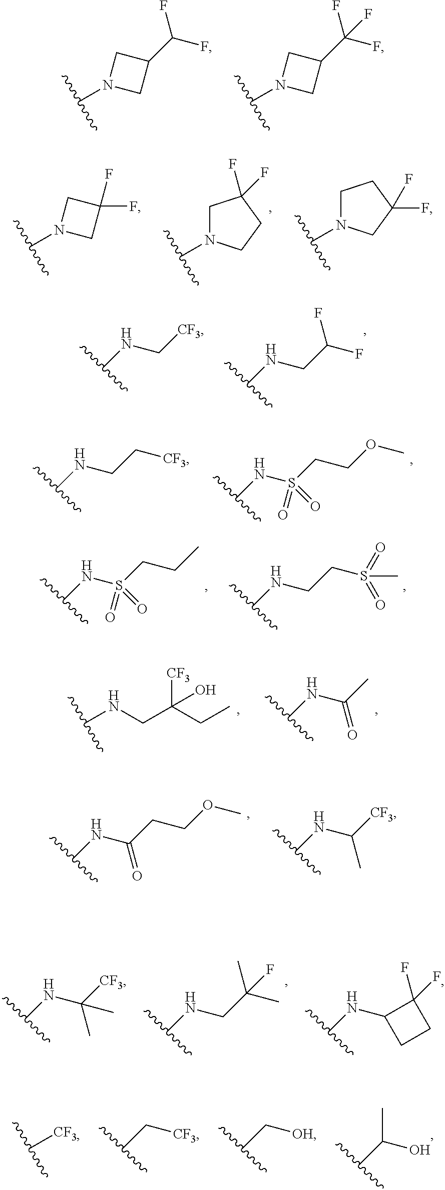

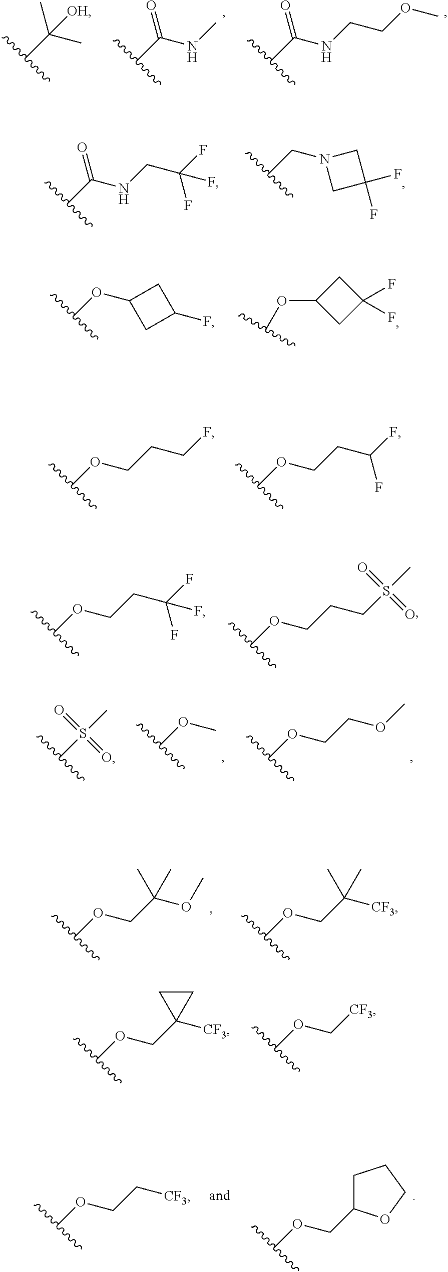

In some cases, each R⁸ is independently selected from —OR²⁰. In some cases, each R⁸ is independently selected from —OR²⁰, wherein R²⁰ is selected C₁₋₆ alkyl, wherein the C₁₋₆ alkyl is substituted with one or more substituents independently selected from halogen, —OH, —CN, —C₁₋₁₀ haloalkyl, —S(O)₂(C₁₋₆ alkyl), —O—C₁₋₁₀ alkyl, C₃-5 carbocycle, and 4- to 5-membered heterocycle. In some cases, each R⁸ is independently selected from

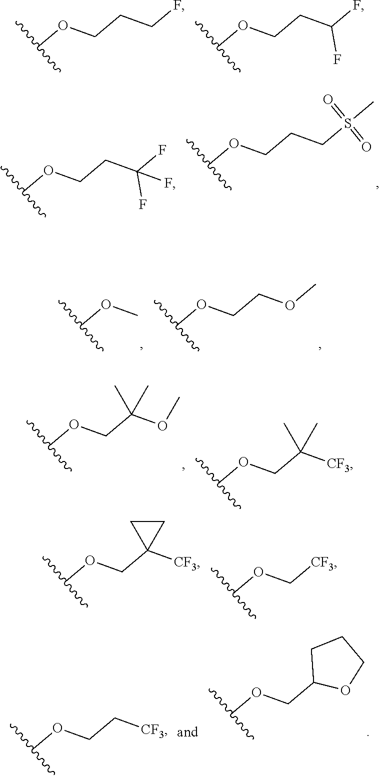

In some cases, each R⁸ is independently selected from —OR²⁰, wherein R²⁰ is selected C₁₋₆ alkyl, wherein the C₁₋₆ alkyl is substituted with one or more substituents independently selected from —O—C₁₋₆ alkyl. In some cases, each R⁸ is independently selected from

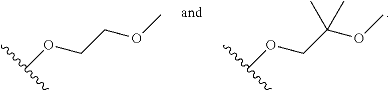

In some cases, each R⁸ is independently selected from

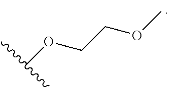 [SMILES: *OCCOC]

In some cases, each R⁸ is independently selected from —OR²⁰, wherein R²⁰ is selected C₁₋₆ alkyl, wherein the C₁₋₆ alkyl is substituted with one or more substituents independently selected from halogen. In some cases, each R⁸ is independently selected from

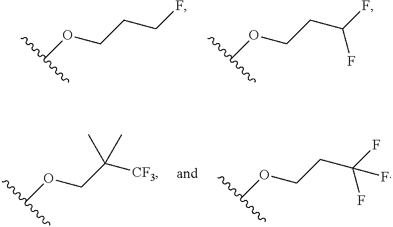

In some cases, each R⁸ is independently selected from —OR²⁰, wherein R²⁰ is selected C₁₋₆ alkyl, wherein the C₁₋₆ alkyl is substituted with one or more substituents independently selected from —S(O)₂(C₁₋₆ alkyl). In some cases, each R⁸ is

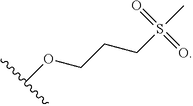 [SMILES: *OCCCS(C)(=O)=O]

In some cases, each R⁸ is independently selected from —OR²⁰, wherein R²⁰ is selected C₁₋₆ alkyl, wherein the C₁₋₆ alkyl is substituted with one or more substituents independently selected from a 5-membered heterocycle. In some cases, the heterocycle has one oxygen atom. In some cases, each R⁸ is

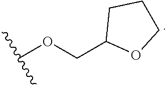 [SMILES: *OCC1CCCO1]

In some embodiments, for a compound or salt of Formula (II), Formula (IID), Formula (IIE), Formula (IIF) or Formula (IIG), R⁷ is selected from C₄-C₇ carbocycle, which is optionally substituted with one or more R⁸. In some cases, R⁷ is selected from C₄-C₆ carbocycle, which is optionally substituted with one or more R⁸. In some cases, R⁷ is selected from

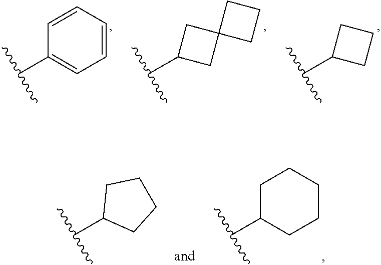

which is optionally substituted with one or more R⁸. In some cases, R⁷ is selected from

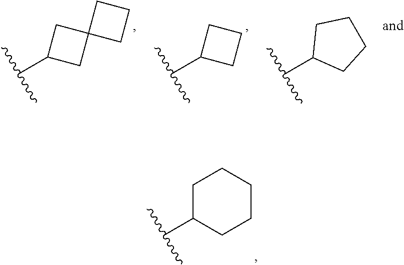 [SMILES: *C1CC2(CCC2)C1 ; *C1CCC1 ; *C1CCCC1 ; *C1CCCCC1]

which is optionally substituted with one or more R⁸. In some cases, R⁷ is selected from

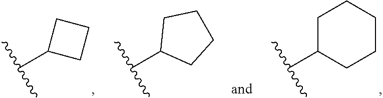 [SMILES: *C1CCC1 ; *C1CCCC1 ; *C1CCCCC1]

which is optionally substituted with one or more R⁸. In some cases, R⁷ is selected from

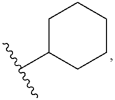 [SMILES: *C1CCCCC1]

which is optionally substituted with one or more R⁸. In some cases, R⁷ is selected from

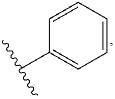 [SMILES: *c1ccccc1]

which is optionally substituted with one or more R⁸. In some cases, each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —C(O)N(R²⁰)₂, —S(O)₂(R²⁰), and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen. In some cases, each R⁸ is independently selected from F, methyl, ethyl,

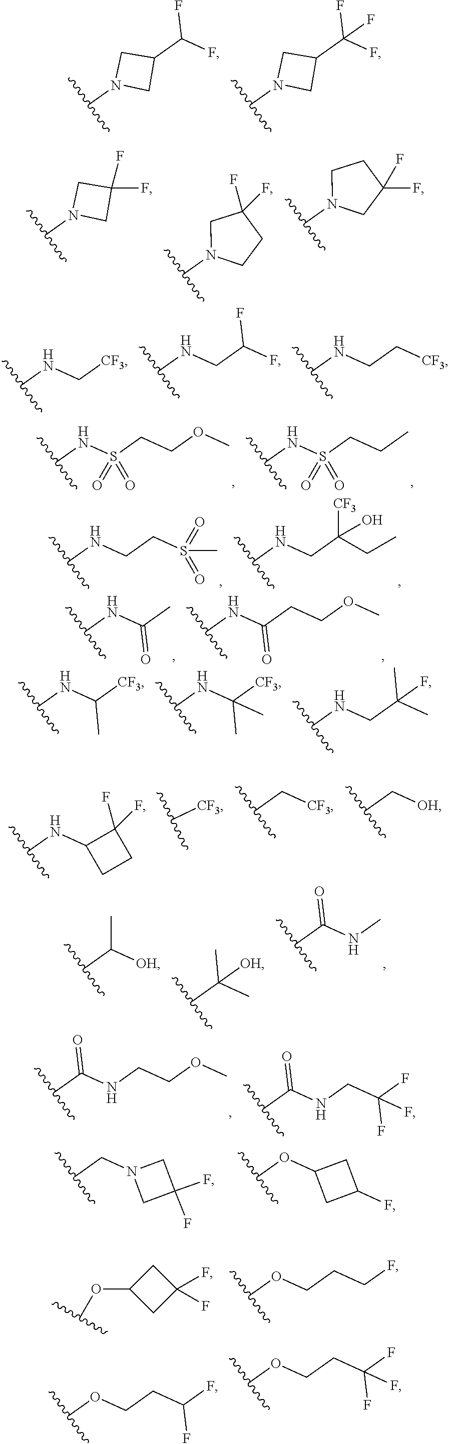

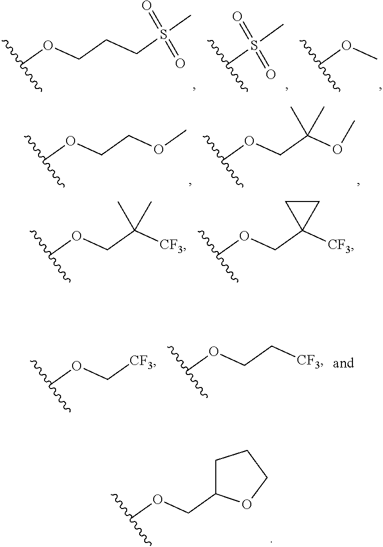

In some cases, R⁷ is

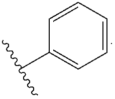 [SMILES: *c1ccccc1]

In some cases, each R⁸ is independently selected from C₁₋₆ hydroxyalkyl, C₁₋₆ alkyl and C₁₋₆ haloalkyl. In some cases, R⁷ is selected from

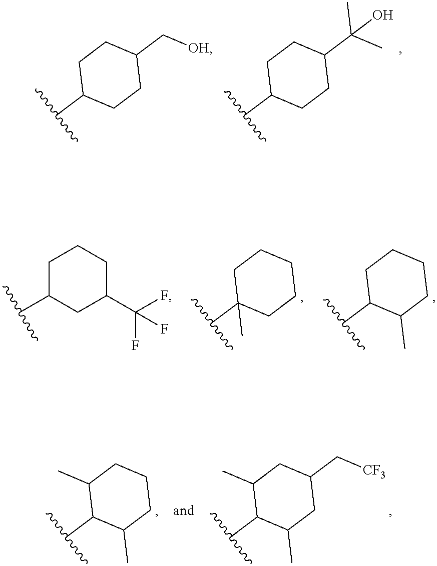

In some cases, each R⁸ is independently selected from C₁₋₆ alkyl. In some cases, R⁷ is selected from.

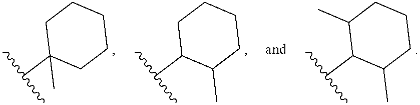

In some embodiments, for a compound or salt of Formula (II). Formula (IID). Formula (IIE). Formula (IIF) or Formula (IIG). R⁷ is

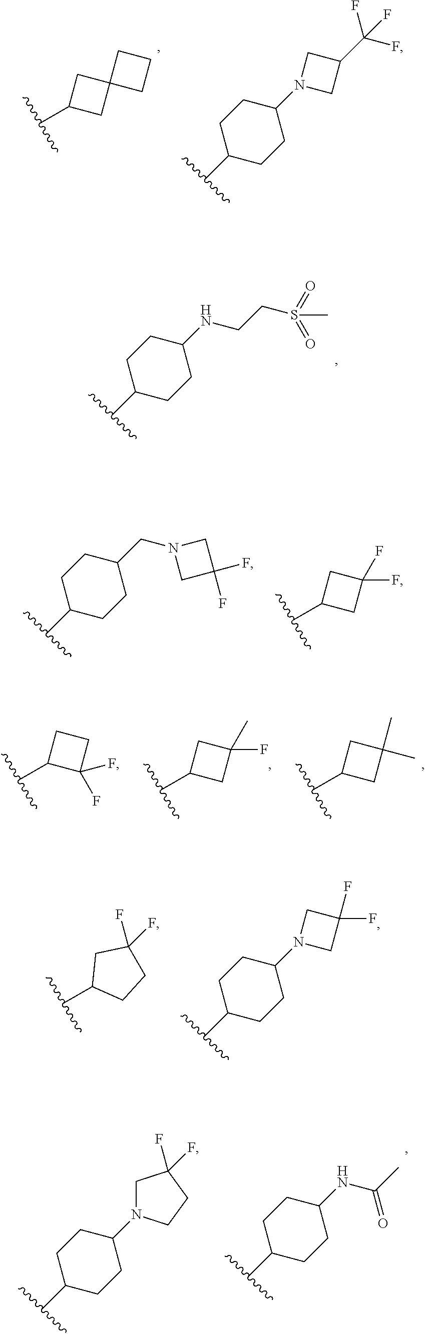

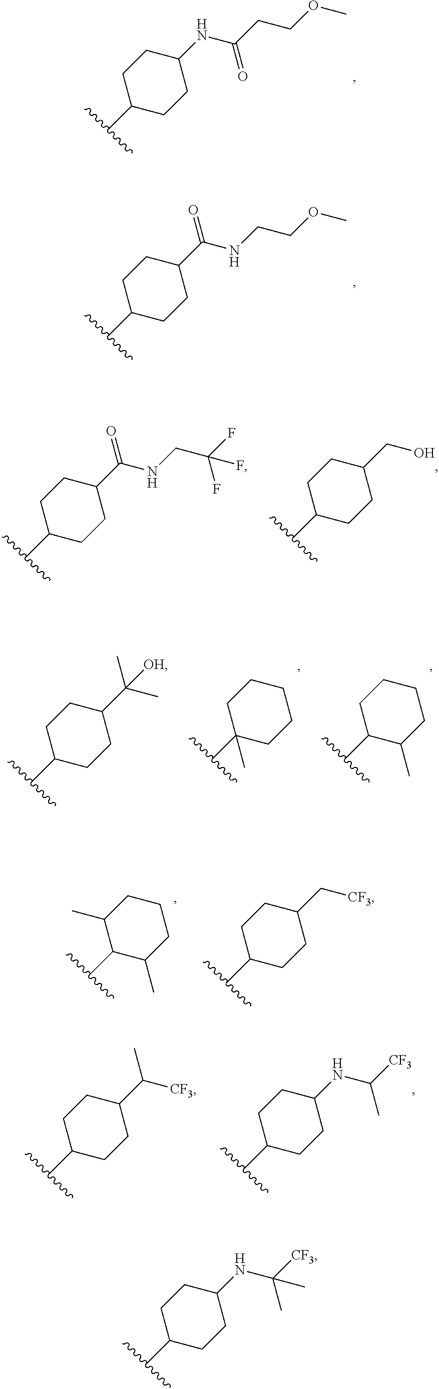

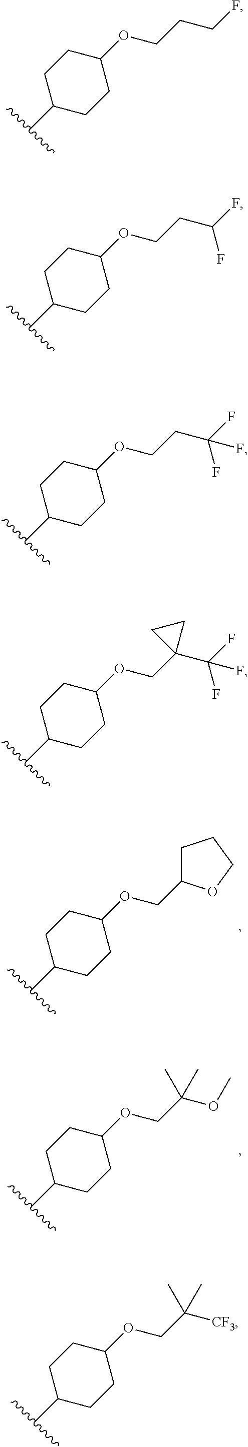

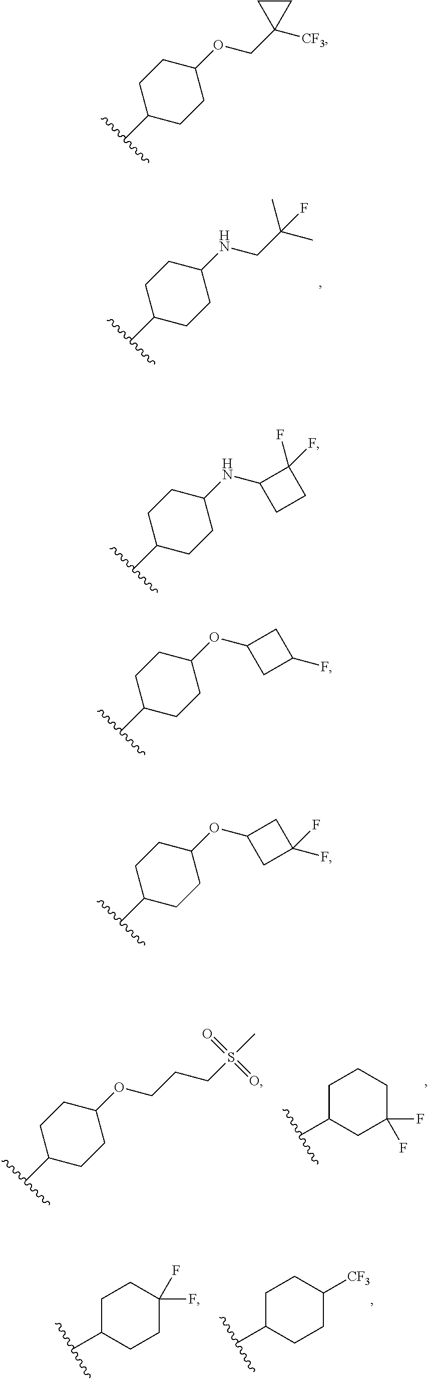

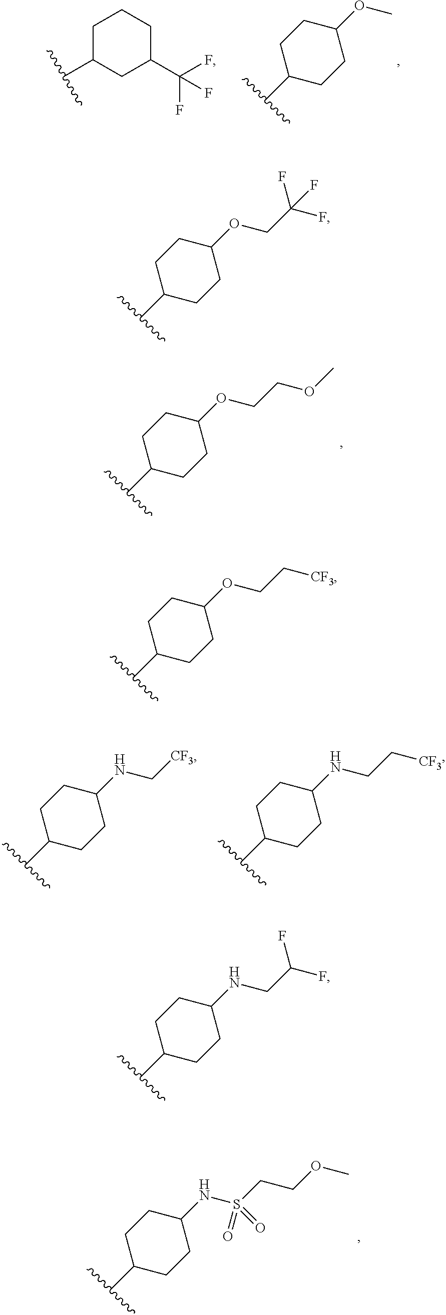

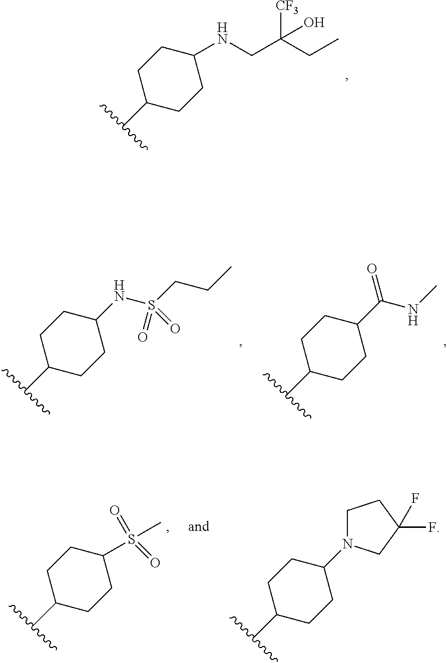 [SMILES: *C1CCC(NCC(O)(CC)C(F)(F)F)CC1 ; *C1CCC(NS(=O)(=O)CCC)CC1 ; *C1CCC(C(=O)NC)CC1 ; *C1CCC(S(C)(=O)=O)CC1 ; *C1CCC(N2CCC(F)(F)C2)CC1]

In some embodiments, for a compound or salt of Formula (II). Formula (IID). Formula (IIE). Formula (IIF) or Formula (IIG). R⁷ is selected from

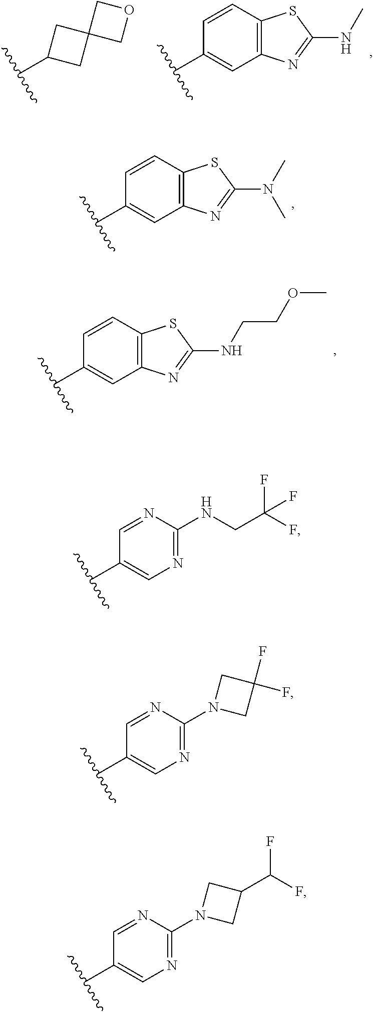

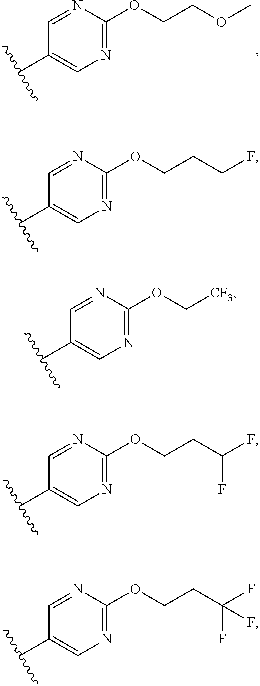 [SMILES: *c1cnc(OCCOC)nc1 ; *c1cnc(OCCCF)nc1 ; *c1cnc(OCC(F)(F)F)nc1 ; *c1cnc(OCCC(F)F)nc1 ; *c1cnc(OCCC(F)(F)F)nc1]

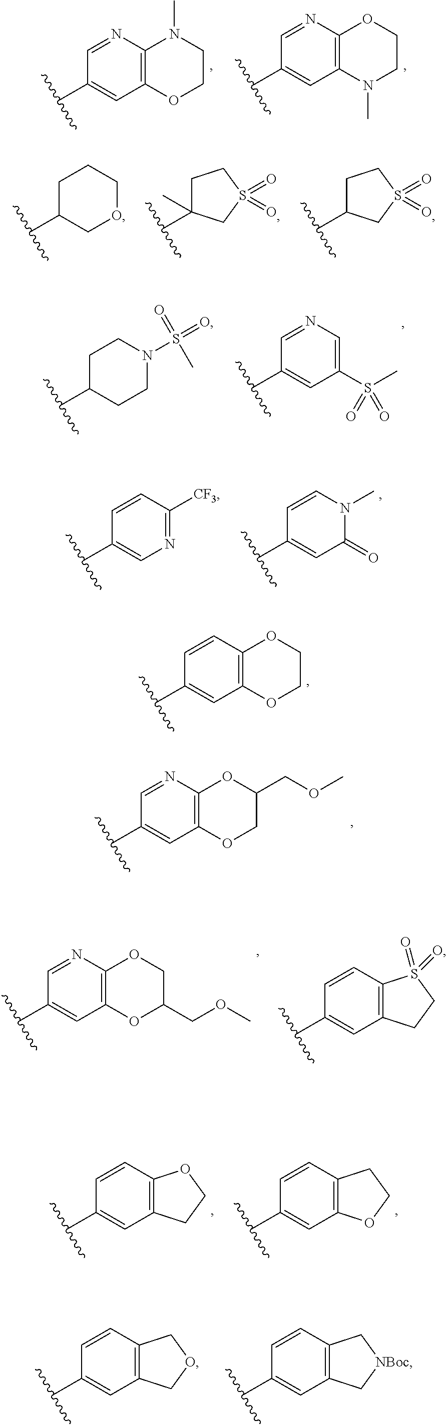

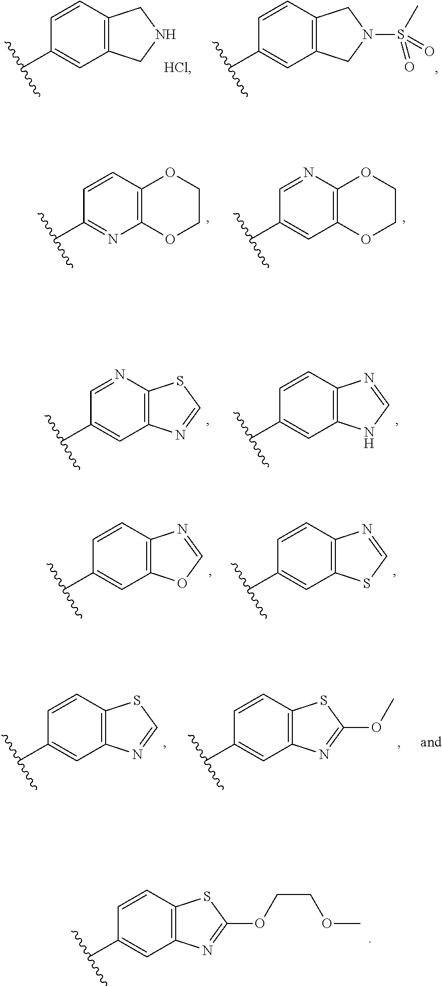

In some embodiments, for a compound or salt of Formula (II), Formula (IIA), Formula (IIB), Formula (IIC), Formula (ID), Formula (IIE), Formula (IIF), or Formula (IIG), R⁷ is

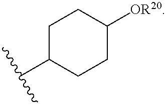

In some cases, R⁷ is selected from

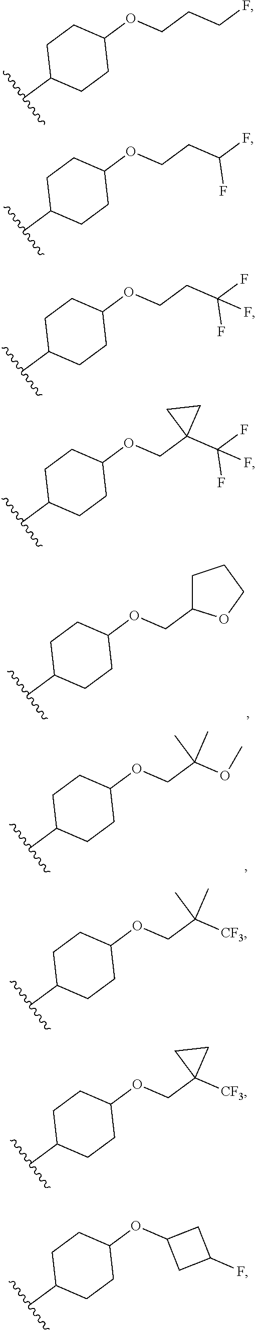

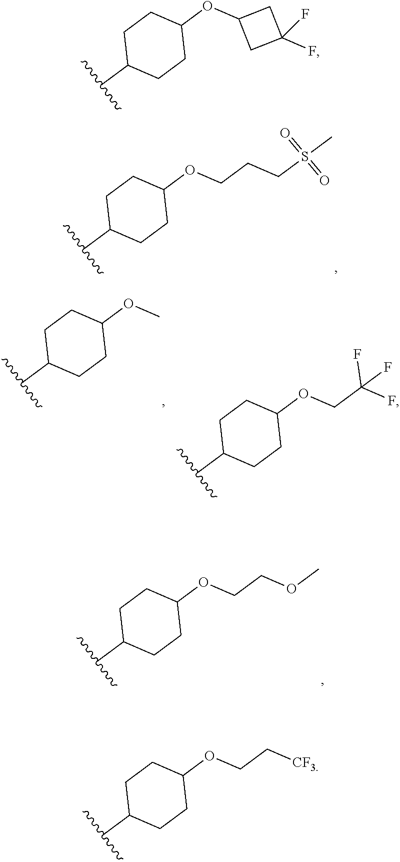

In some cases, R⁷ is

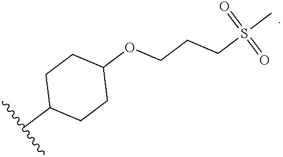 [SMILES: *C1CCC(OCCCS(C)(=O)=O)CC1]

In some cases, R⁷ is selected from

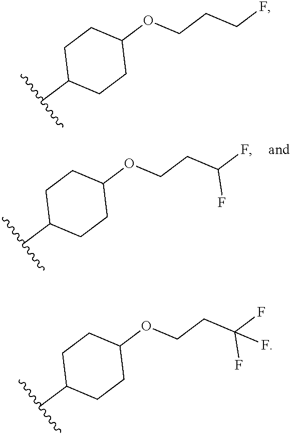 [SMILES: *C1CCC(OCCCF)CC1 ; *C1CCC(OCCC(F)F)CC1 ; *C1CCC(OCCC(F)(F)F)CC1]

In some cases, R⁷ is

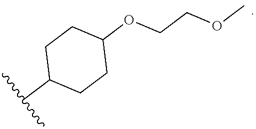 [SMILES: *C1CCC(OCCOC)CC1]

In some cases, R⁷ is

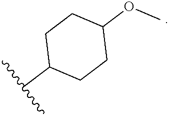 [SMILES: *C1CCC(OC)CC1]

In some cases, R⁷ is

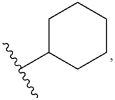 [SMILES: *C1CCCCC1]

which is substituted with at least one R⁸ selected from C₁₋₆ alkyl, C₁₋₆ hydroxyalkyl and C₁₋₆ haloalkyl. In some cases, R⁷ is selected from

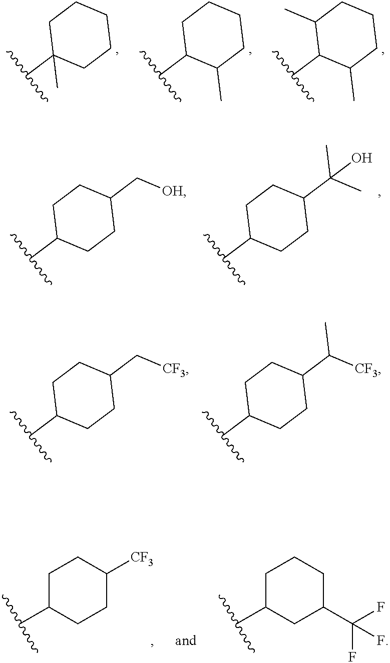

In some cases, R⁷ is

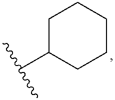 [SMILES: *C1CCCCC1]

which is substituted with at least one R⁸ selected from —S(O)₂(R²⁰). In some cases, R⁷ is

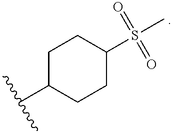 [SMILES: *C1CCC(S(C)(=O)=O)CC1]

In some cases, R⁷ is

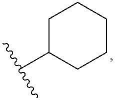 [SMILES: *C1CCCCC1]

which is substituted with at least one R⁸ selected from —N(R²⁰)₂. In some cases, R⁷ is selected from

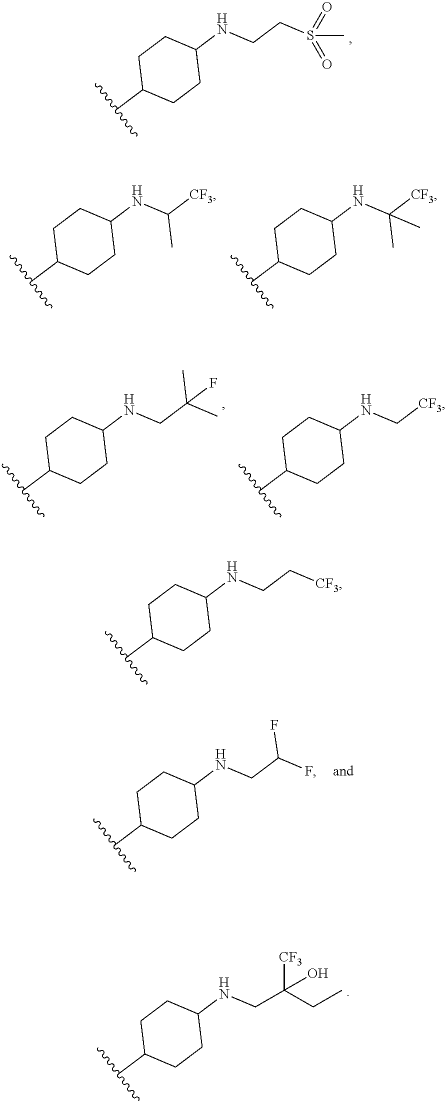

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), R⁷ is selected from

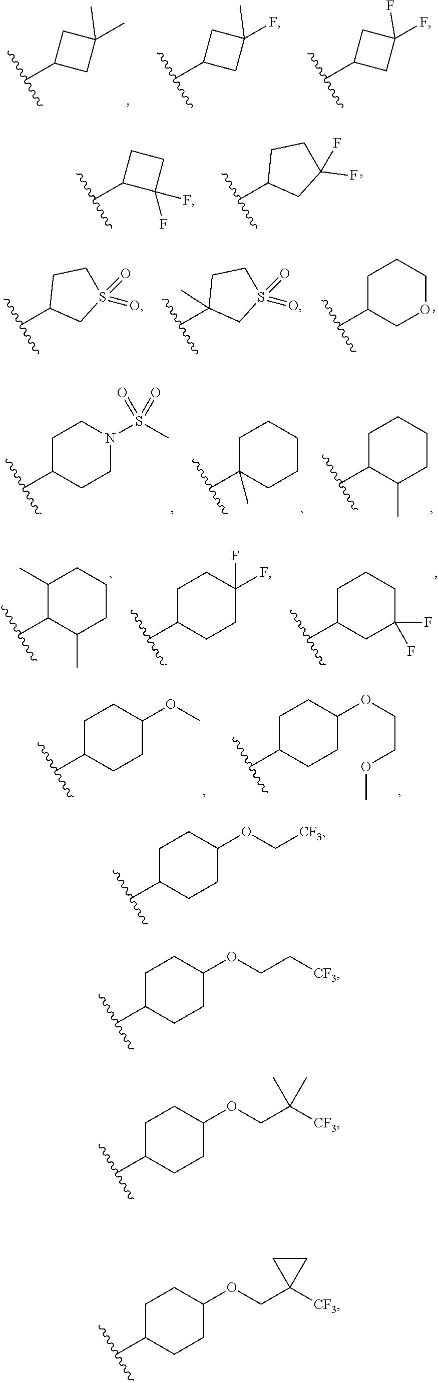

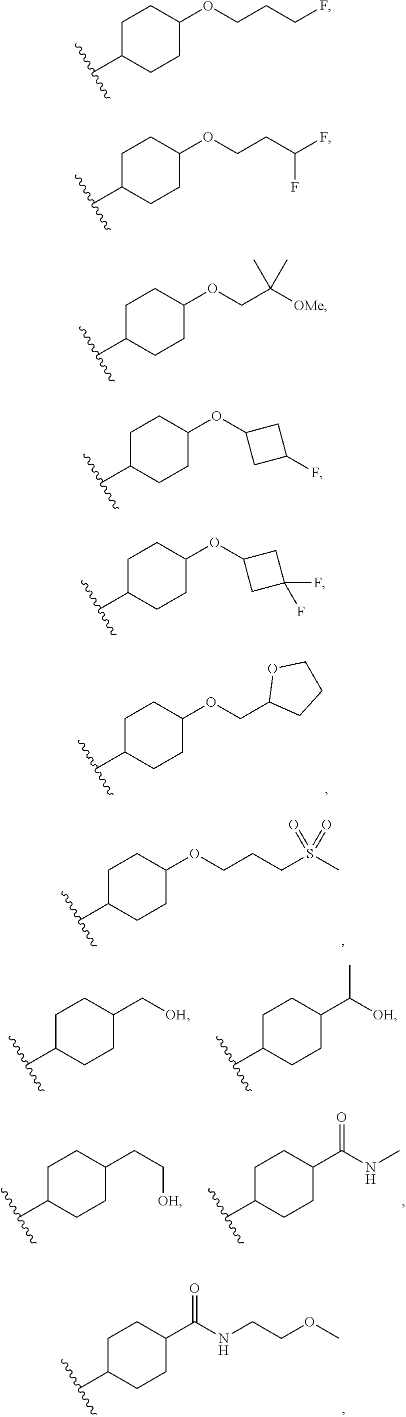

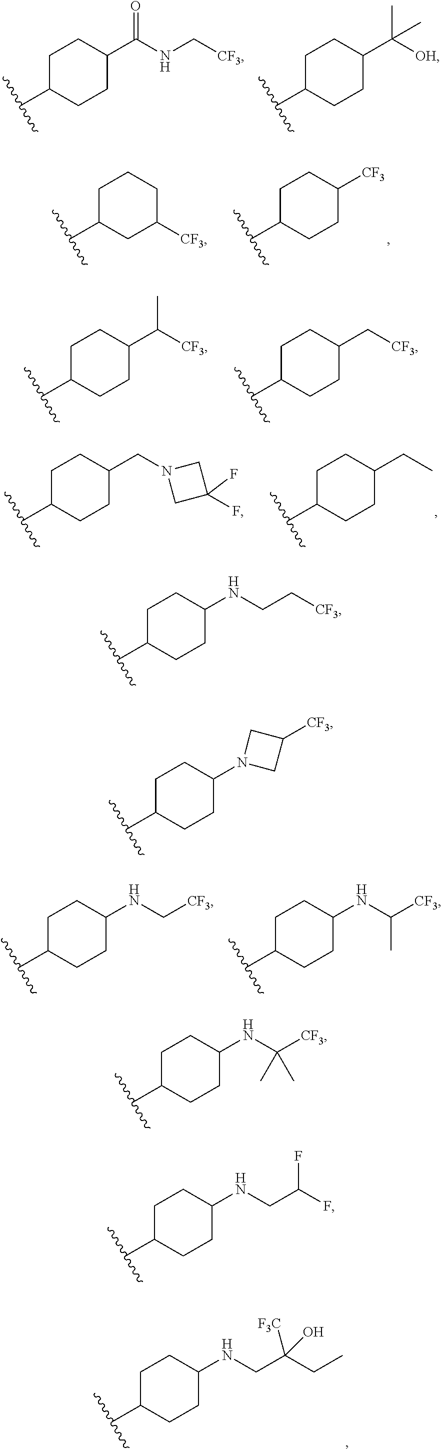

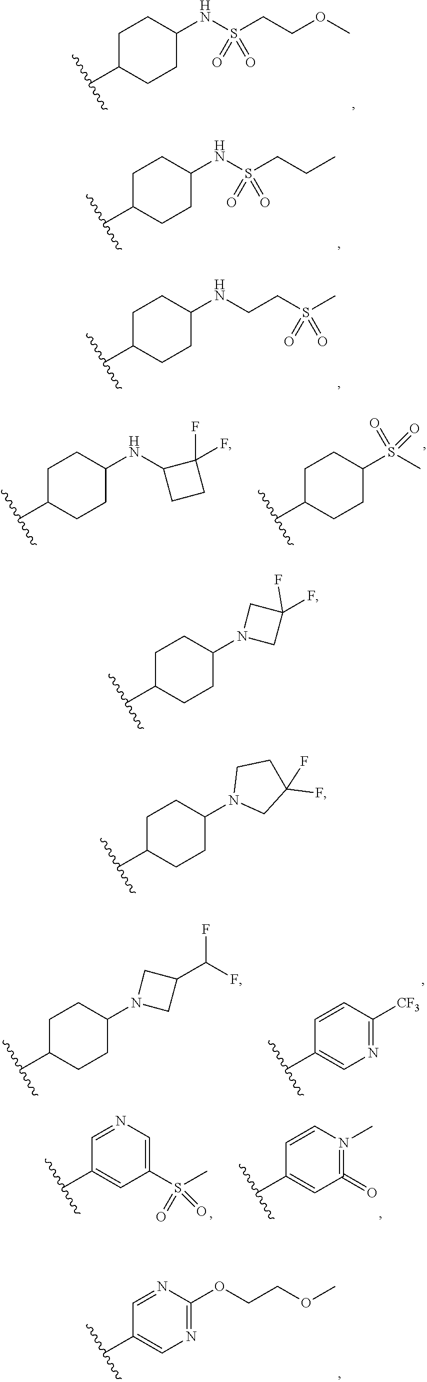

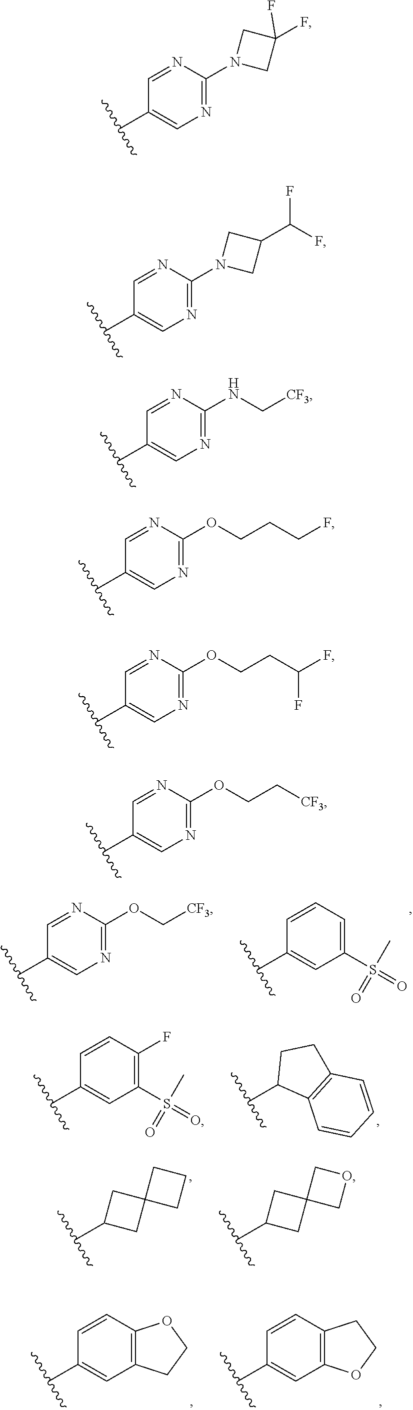

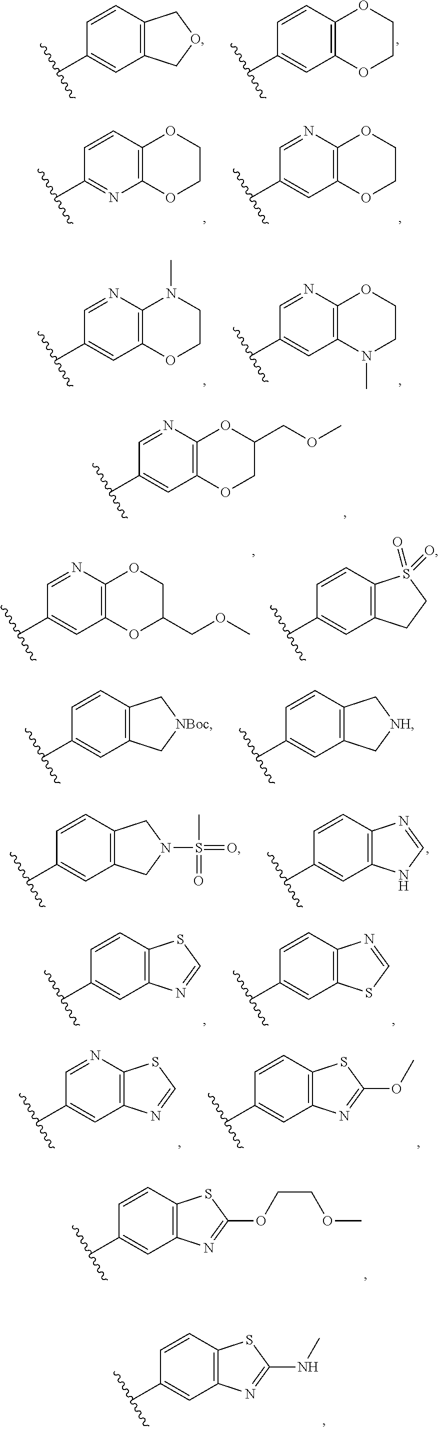

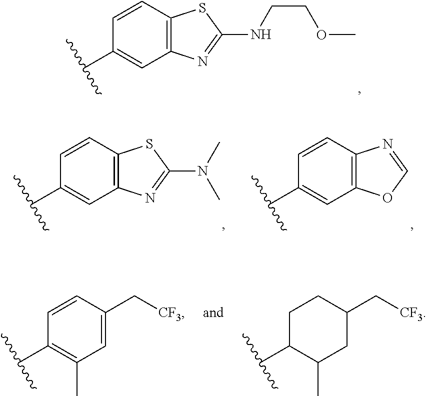 [SMILES: *c1ccc2sc(NCCOC)nc2c1 ; *c1ccc2sc(N(C)C)nc2c1 ; *c1ccc2ncoc2c1 ; *c1ccc(CC(F)(F)F)cc1C ; *C1CCC(CC(F)(F)F)CC1C]

In some cases. R⁷ is selected from

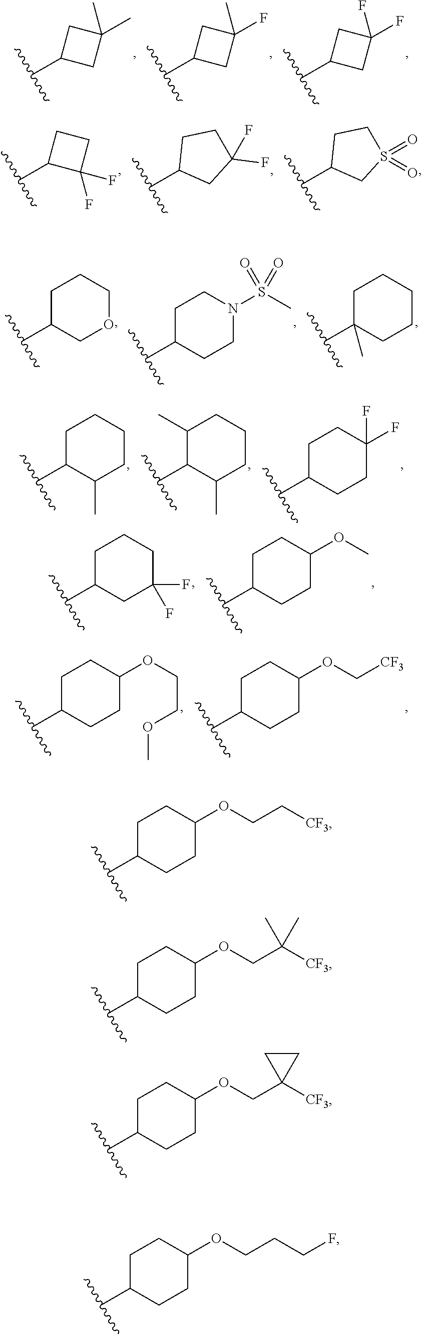

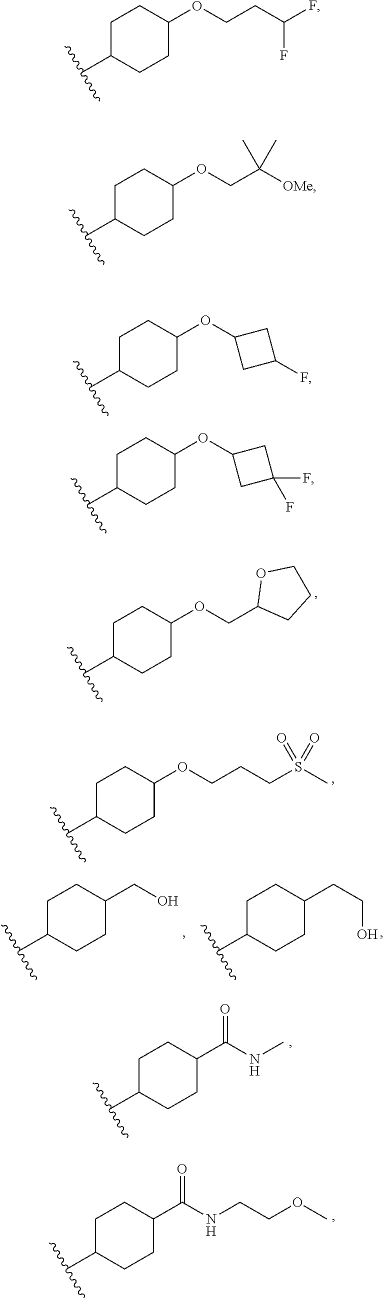

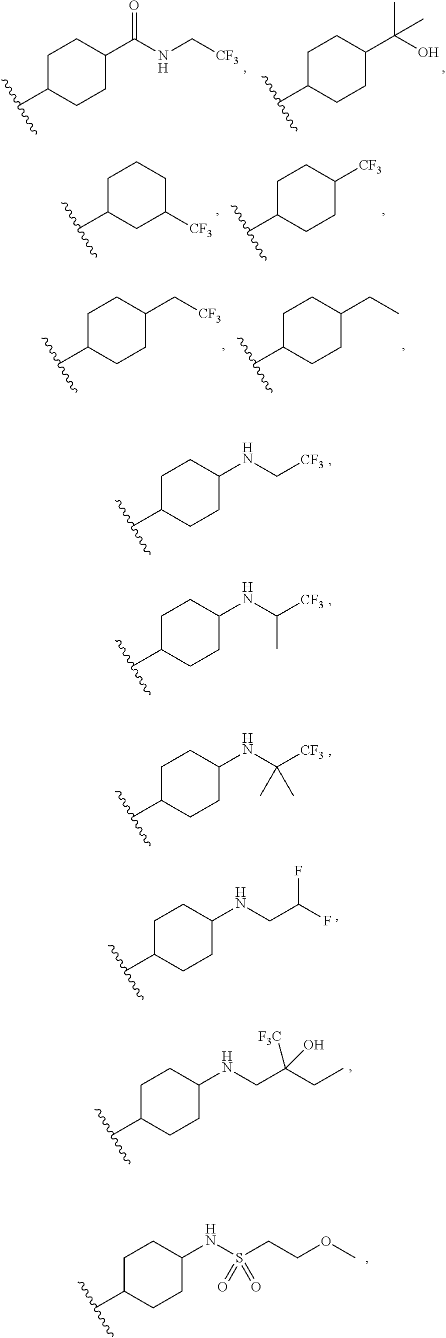

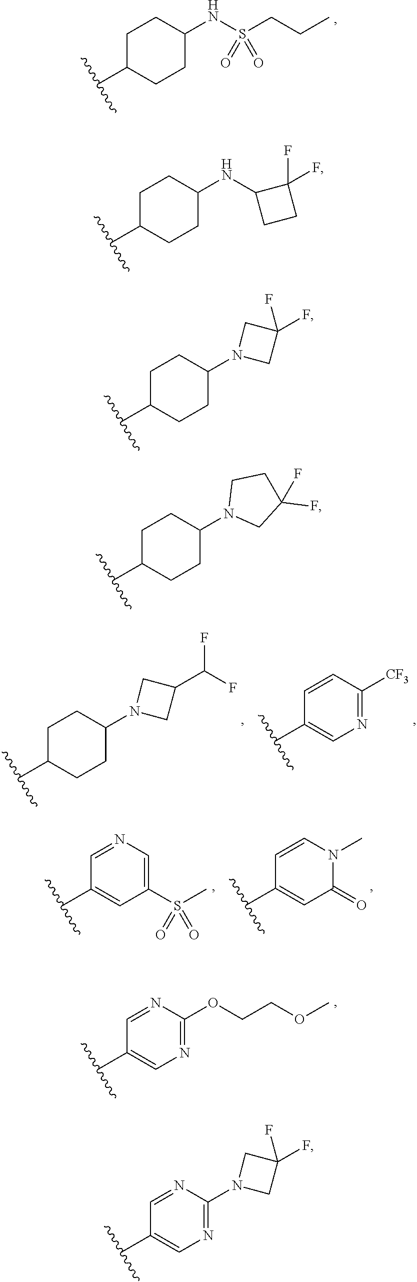

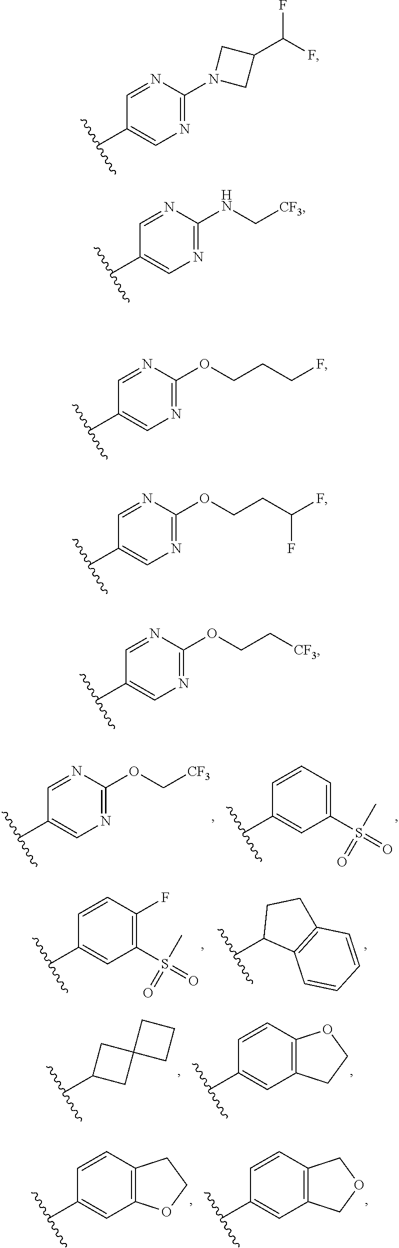

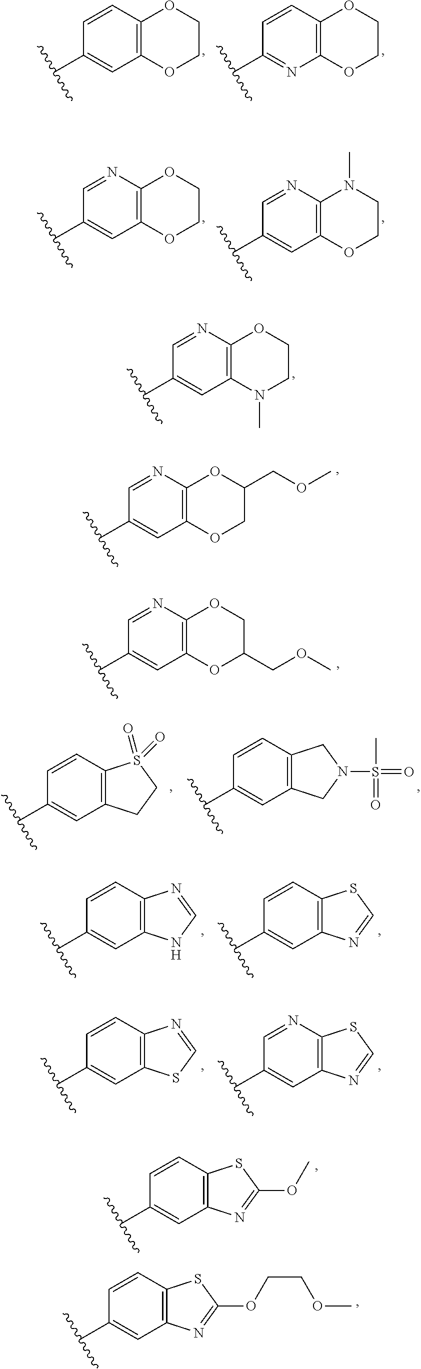

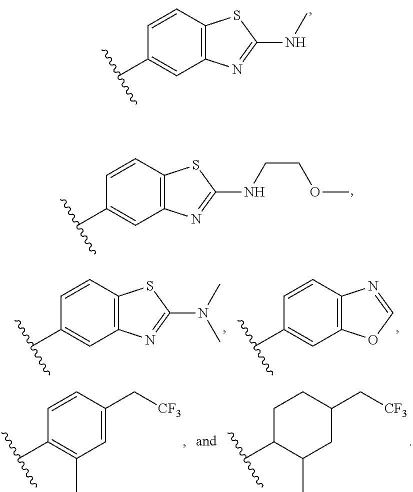

In some cases. R⁷ is selected from

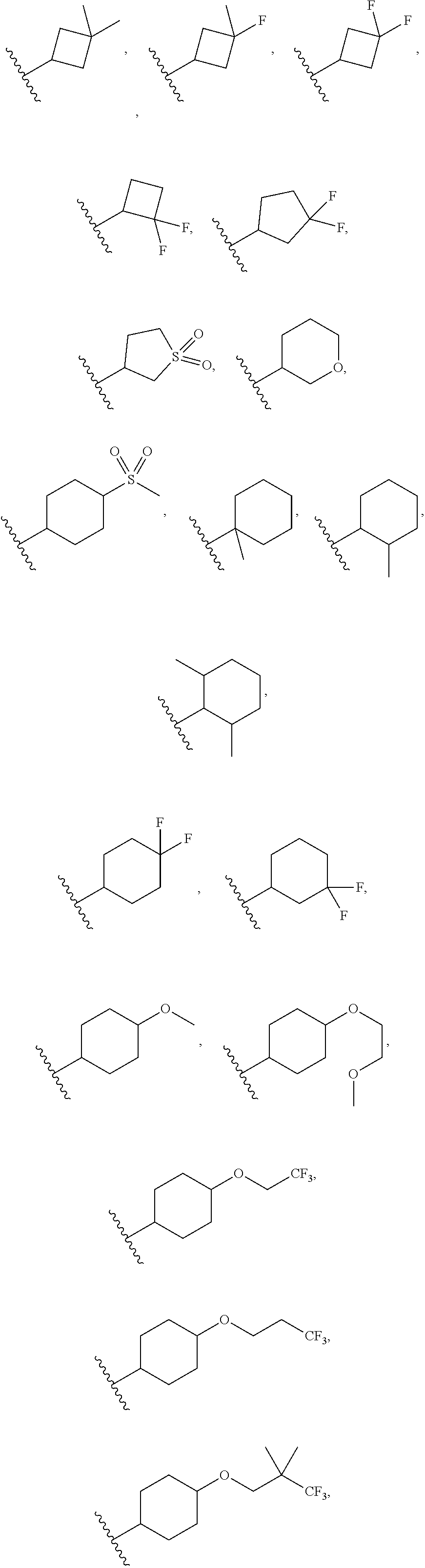

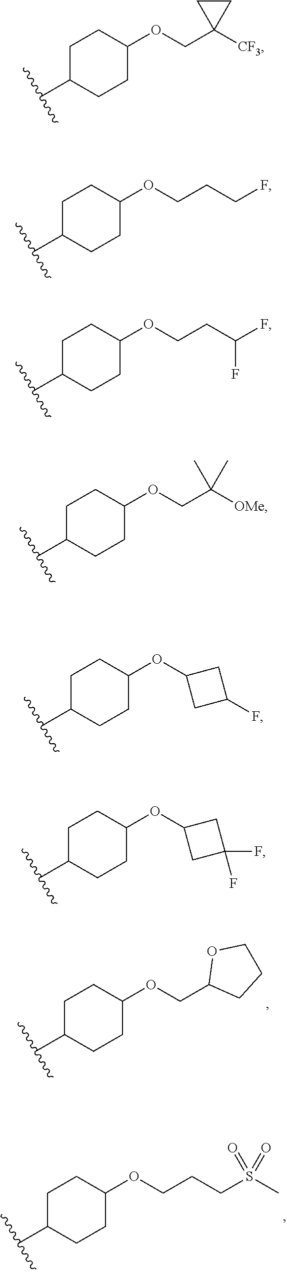

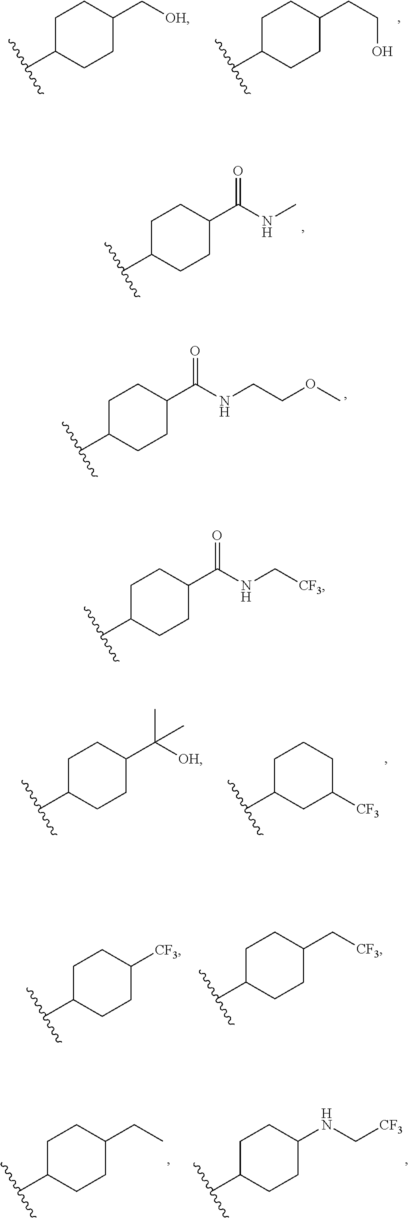

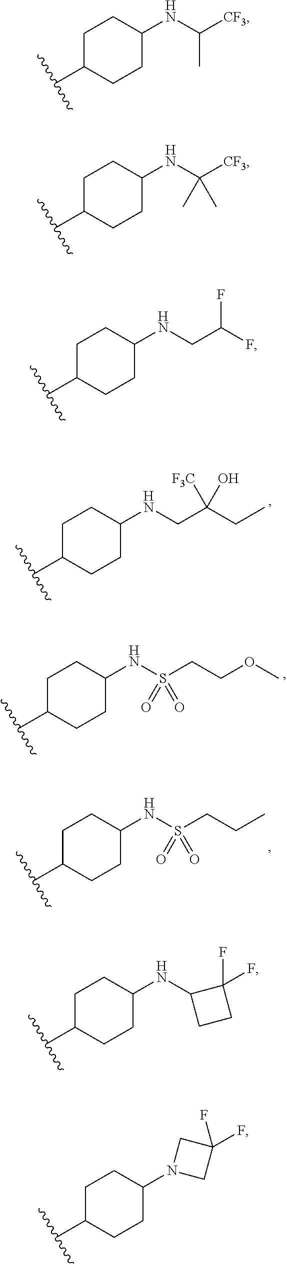

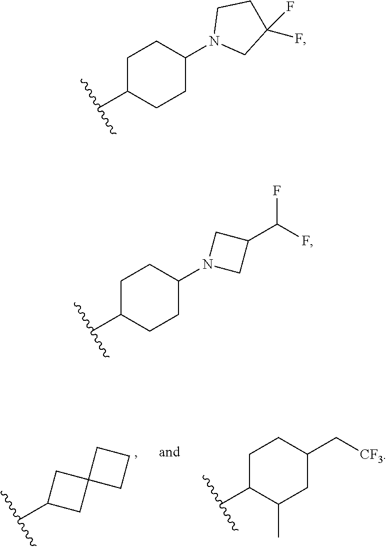 [SMILES: *C1CCC(N2CCC(F)(F)C2)CC1 ; *C1CCC(N2CC(C(F)F)C2)CC1 ; *C1CC2(CCC2)C1 ; *C1CCC(CC(F)(F)F)CC1C]

In some cases. R⁷ is selected from

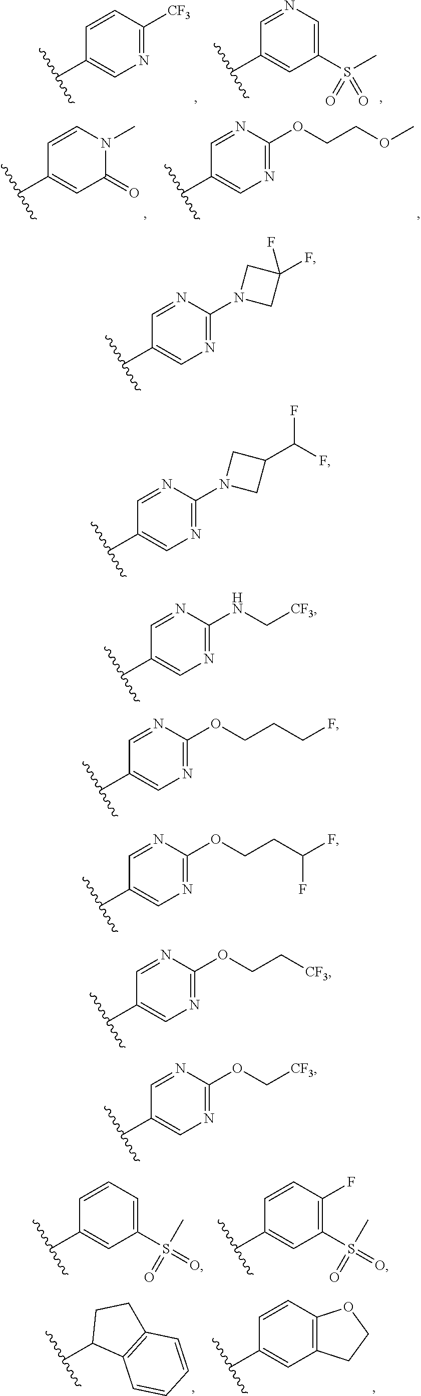

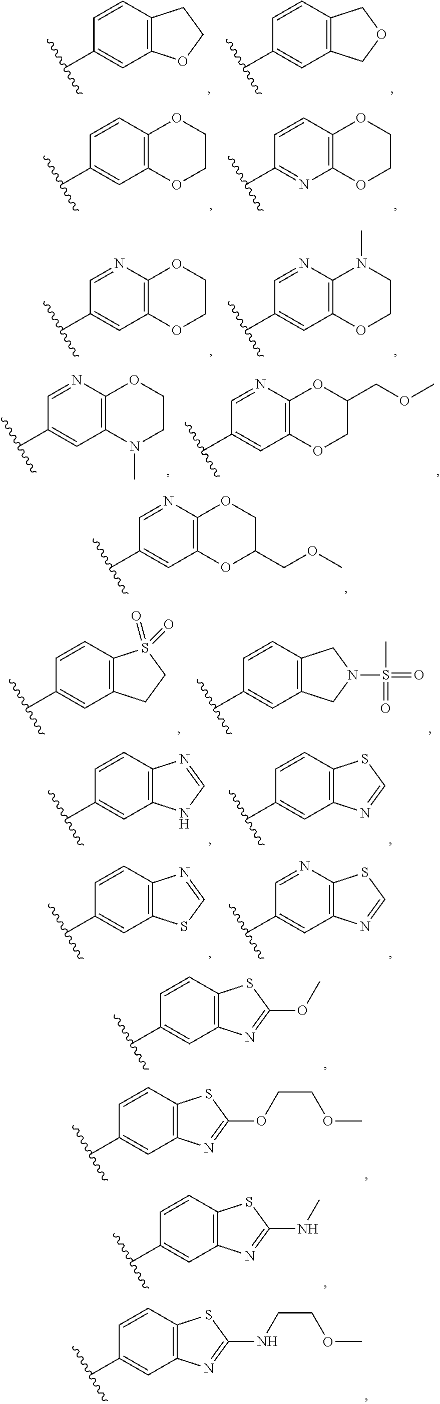

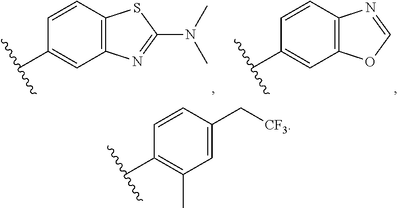

In some cases. R⁷ is selected from

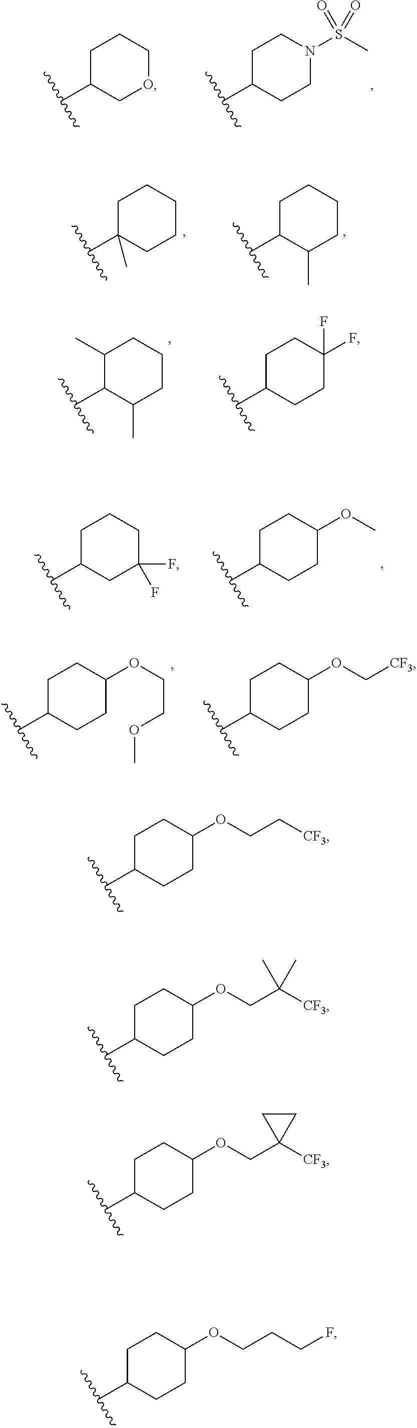

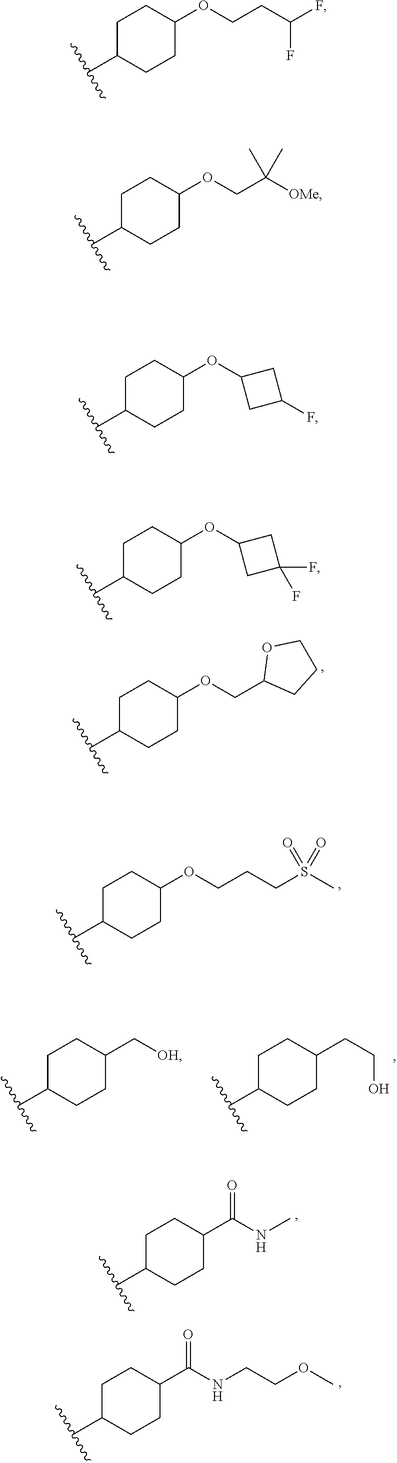

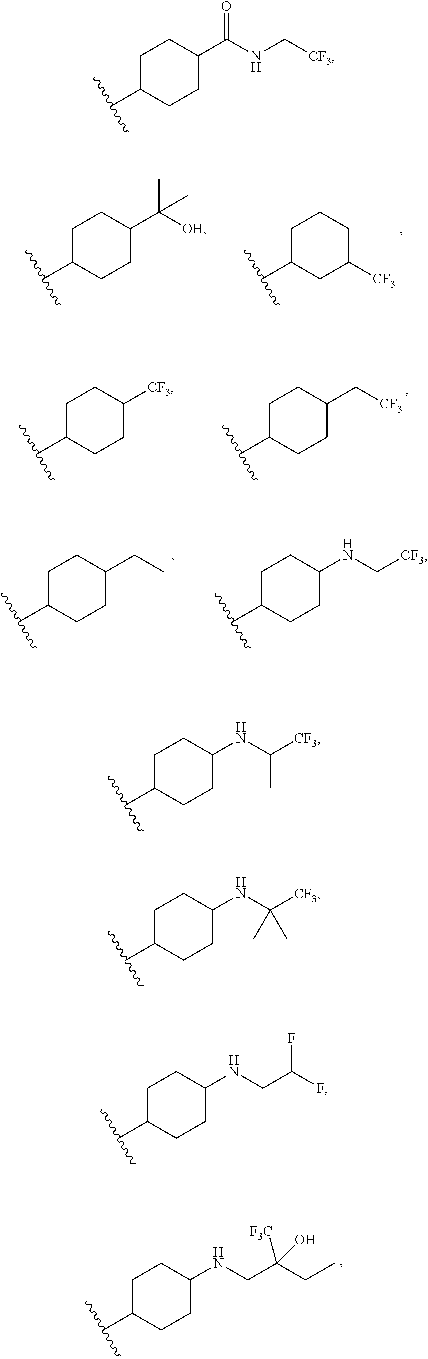

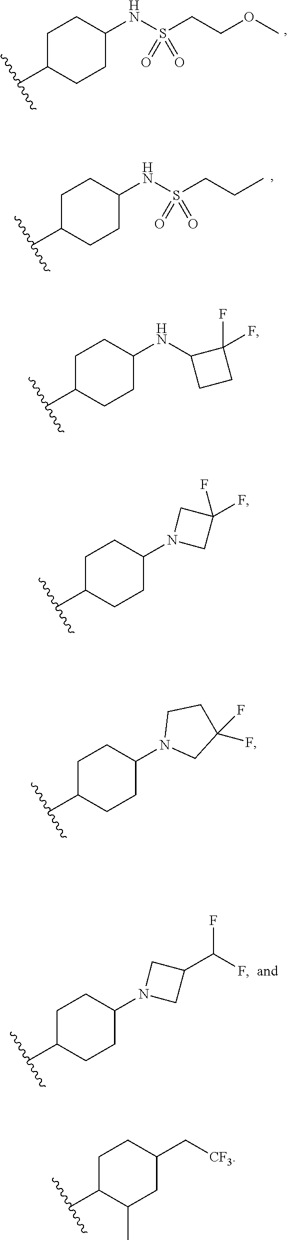

In some embodiments, for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), each R⁸ is independently selected from fluoro,

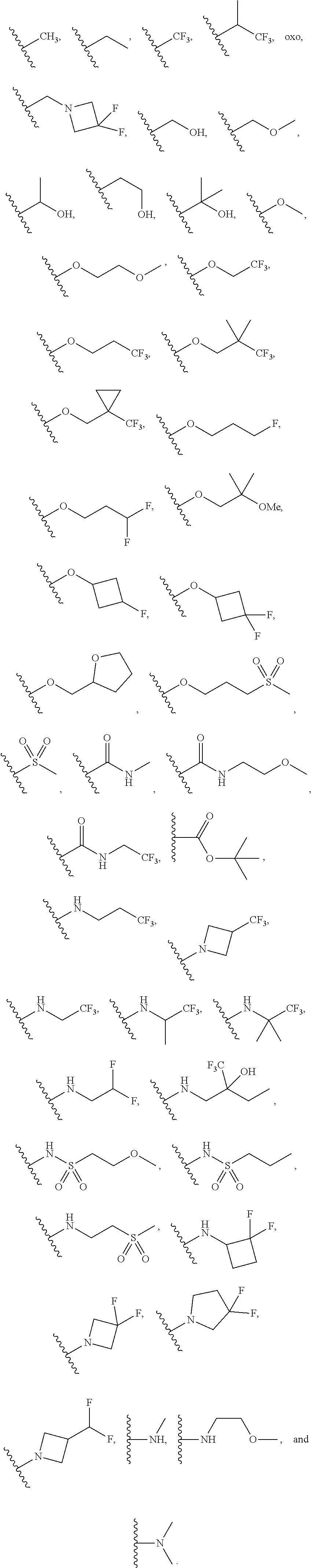

In some cases, each R⁸ is independently selected from fluoro,

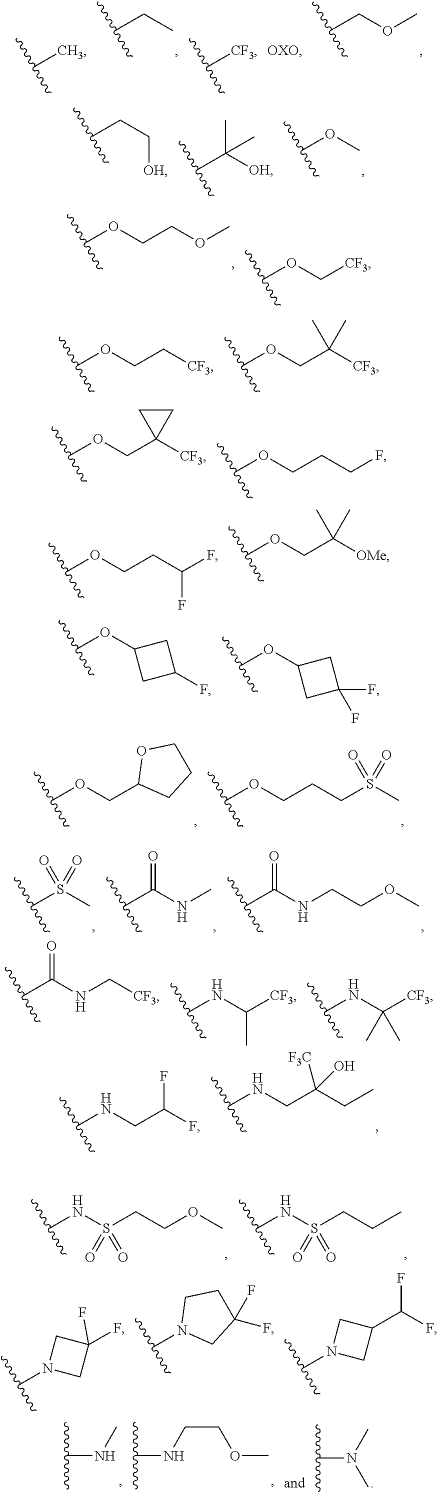

In some cases, each R⁸ is independently selected from fluoro,

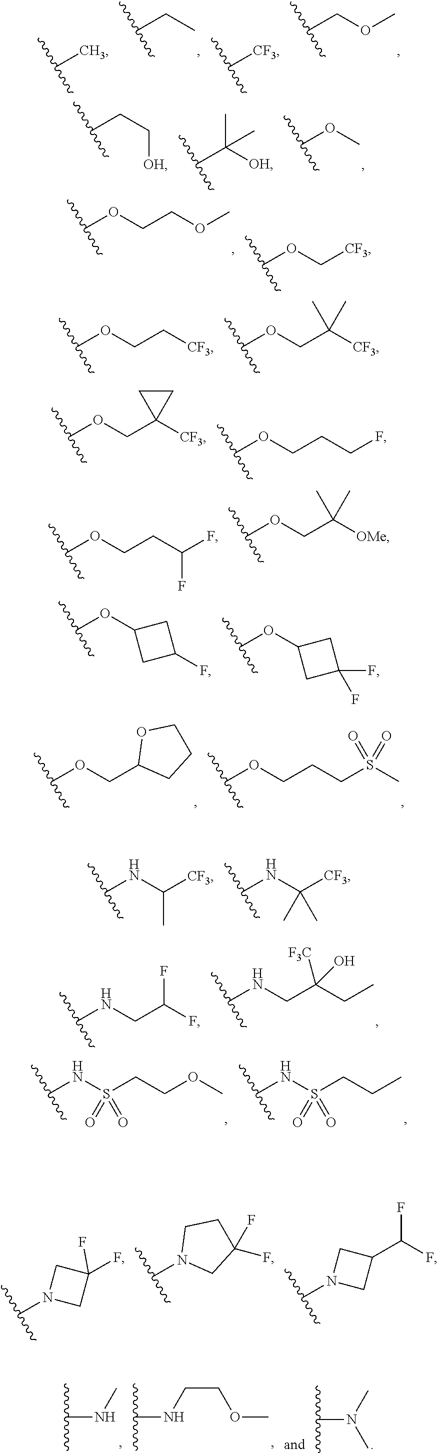

In some cases, each R⁸ is independently selected from fluoro,

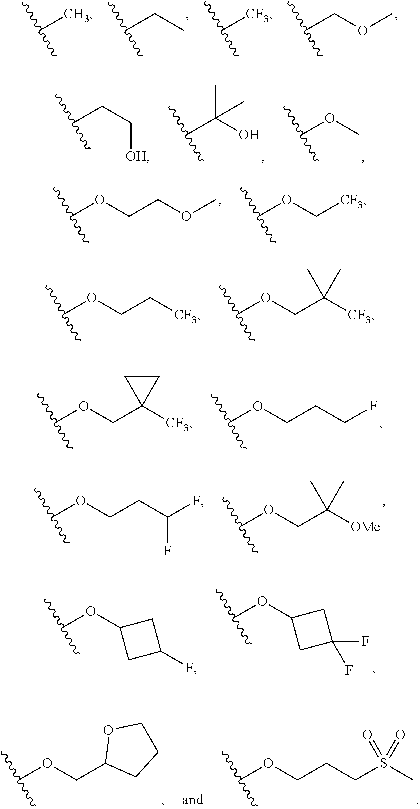

In some cases, each R⁸ is independently selected from fluoro,

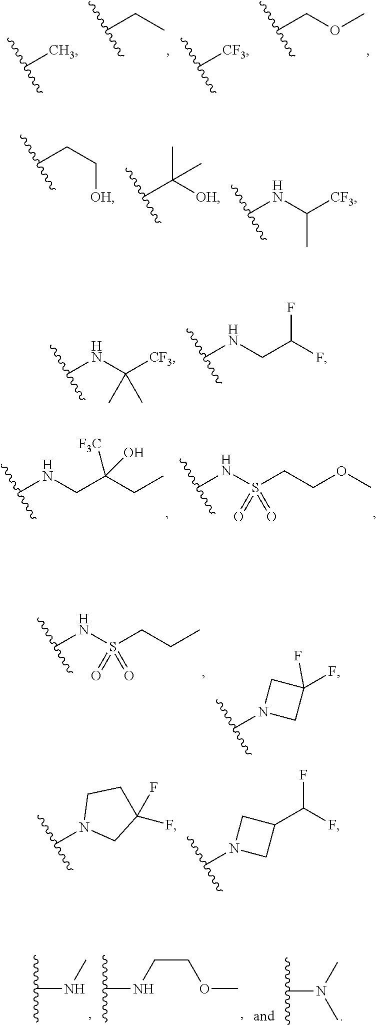

In some embodiments, for a compound or salt of Formula (II), (IIA), (IIB), (IIC), A is selected from N and CR¹⁸. In some cases, D is selected from N and CR¹⁹. In some cases, A is N and D is N. In some cases, A is N and D is CR¹⁹. In some cases, A is CR¹⁸ and D is selected from N. In some cases, A is CR¹⁸ and D is selected from CR¹⁹. In some cases, R¹⁷ is selected from hydrogen, halogen, —OMe, C₁₋₆ haloalkyl, C₁₋₆ alkyl, and C₃ carbocycle. In some cases, R¹⁸ is selected from hydrogen and halogen. In some cases, R¹⁹ is selected from hydrogen, and halogen.

In some embodiments, for a compound or salt of Formula (II), Formula (IIA), Formula (IIB), Formula (IIC), Formula (IID), Formula (IIE), Formula (IIF), or Formula (IIG), substituents are selected from compounds of the example section.

In some embodiments, a compound of Formula (II), Formula (IIA), or Formula (IID) is selected from

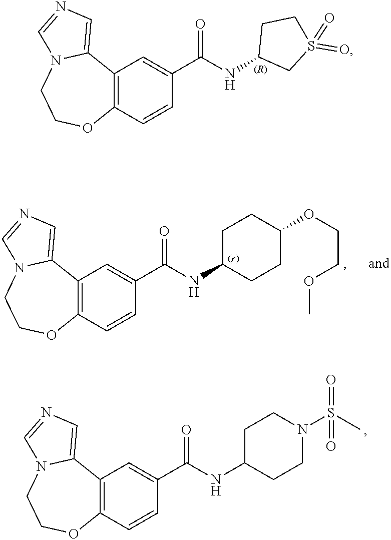

or a pharmaceutically acceptable salt of any one thereof.

Chemical entities having carbon-carbon double bonds or carbon-nitrogen double bonds may exist in Z—or E-form (or cis- or trans-form). Furthermore, some chemical entities may exist in various tautomeric forms. Unless otherwise specified, compounds described herein are intended to include all Z—, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and(S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

When stereochemistry is not specified, molecules with stereocenters described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, compositions of the disclosure may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113 (3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

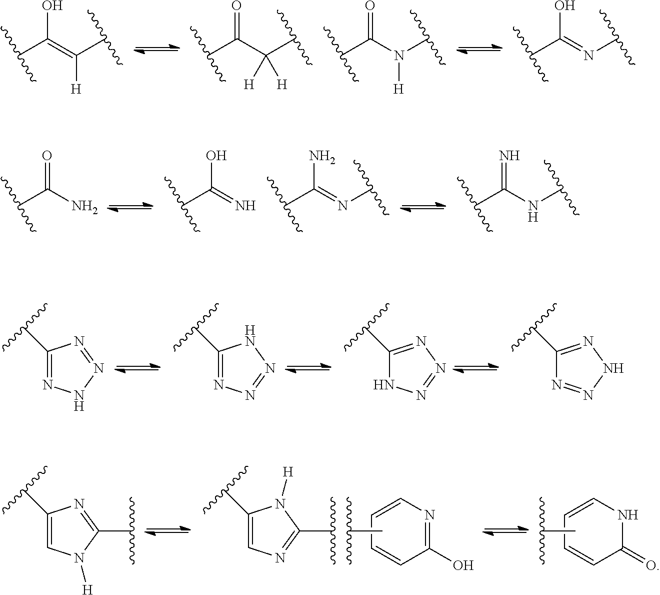

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean. Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma. Rajender S. The Synthesis of Radiolabeled Compounds via Organometallic Intermediates. Tetrahedron, 1989, 45(21), 6601-21; and Evans. E. Anthony. Synthesis of radiolabeled compounds. J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269: G210-218 (1995): McLoed et al., Gastroenterol, 106:405-413 (1994); Hochhaus et al., Biomed. Chrom . . . 6:283-286 (1992): J. Larsen and H. Bundgaard, Int. J. Pharmaceutics. 37, 87 (1987): J. Larsen et al., Int. J. Pharmaceutics, 47, 103 (1988); Sinkula et al., J. Pharm. Sci., 64:181-210 (1975): T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989): T. W. Greene and P. G. M. Wuts, Protective Groups in Organic Synthesis. 2d. Ed. (1991): L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

A compound or salt of any one of the Formulas or sub Formulas described herein (e.g., Formula (Formula (I), Formula (IA), Formula (IB), Formula (IC), or Formula (ID), etc.,) may be formulated in any suitable pharmaceutical formulation. A pharmaceutical formulation of the present disclosure typically contains an active ingredient (e.g., compound or salt of any one of the Formulas described herein) and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

In certain embodiments, a compound or salt of any one of the Formulas or sub Formulas described herein is formulated with an agent that inhibits degradation of the compound or salt. In certain embodiments, the compound or salt is formulated with one or more antioxidants. Acceptable antioxidants include, but are not limited to, citric acid, d,I-α-tocopherol, BHA, BHT, monothioglycerol, ascorbyl palmitate, ascorbic acid, and propyl gallate. In certain embodiments, the formulation contains from 0.1 to 30%, from 0.5 to 25%, from 1 to 20%, from 5 to 15%, or from 7 to 12% (wt/wt) CCI-779, from 0.5 to 50%, from 1 to 40%, from 5 to 35%, from 10 to 25%, or from 15 to 20% (wt/wt) water soluble polymer, from 0.5 to 10%, 1 to 8%, or 3 to 5% (wt/wt) surfactant, and from 0.001% to 1%, 0.01% to 1%, or 0.1% to 0.5% (wt/wt) antioxidant. In certain embodiments, the antioxidants of the formulations of this invention will be used in concentrations ranging from 0.001% to 3% wt/wt.

In certain embodiments, a compound or salt of any one of the Formulas or sub Formulas described herein is formulated with a pH modifying agent to maintain a pH of about 4 to about 6. Acceptable pH modifying agents include, but are not limited to citric acid, sodium citrate, dilute HCl, and other mild acids or bases capable of buffering a solution containing a compound or a salt of the disclosure to a pH in the range of about 4 to about 6.

In certain embodiments, a compound or salt of any one of the Formulas or sub Formulas described herein is formulated with a chelating agent or other material capable of binding metal ions, such as ethylene diamine tetra acetic acid (EDTA) and its salts are capable of enhancing the stability of a compound or salt of any one of the Formulas described herein.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of a compound or salt of any one of the Formulas or sub Formulas described herein will be dependent on the mammal being treated, the severity of the disorder or condition, the rate of administration, the disposition of the compound or salt of any one of the Formulas or sub Formulas described herein and the discretion of the prescribing physician.

In some embodiments, pharmaceutically acceptable carriers of a compound or salt of any one of the Formulas or sub Formulas described herein can include a physiologically acceptable compound that is an antioxidant.

In some embodiments, the disclosure provides a pharmaceutical composition for oral administration containing at least one compound or salt of any one of the Formulas or sub Formulas described herein and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of the Formulas or sub Formulas described herein moistened with an inert liquid diluent.

In some embodiments, the disclosure provides a pharmaceutical composition for injection containing a compound or salt of any one of the Formulas described herein and a pharmaceutical excipient suitable for injection. Components and amounts of agents in the composition are as described herein.

In certain embodiments, the compound or salt of any one of the Formulas or sub Formulas described herein may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of the Formulas or sub Formulas described herein and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

The disclosure also provides kits. The kits may include a compound or salt of any one of the Formulas or sub Formulas described herein and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, a compound or salt of any one of the Formulas or sub Formulas described herein and the agent are provided as separate compositions in separate containers within the kit. In some embodiments, a compound or salt of any one of the Formulas or sub Formulas described herein and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Therapeutic Applications

In one aspect, the present disclosure provides a method of inhbiting CD38 comprising administering a compound or salt of for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), In some cases, the method comprises inhibiting CD38 by administering a pharmaceutical composition comprising a compound or salt of for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), and a pharmaceutically acceptable excipient with any one thereof.

In some aspects, the present disclosure provides a method of treating a disease or disorder. The methods comprises administering a compound or salt disclosed here or a pharmaceutical composition to a subject in need thereof. The disease or disorder is selected from: neurodegenerative disease, type I diabetes, insulin resistance, Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra, Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, cystic fibrosis, pancreatitis, idiopathic pulmonary fibrosis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia-telangiectasia, rheumatoid arthritis, lupus, alcohol intolerance, hyperphosphatemia, acute lung injury, and ARDS. In some cases, the disease or disorder is a neurodegenerative disease. In some cases, the disease or disorder is muscular dystrophy. In some cases, the disease or disorder is a metabolic disorder. In some cases, the disease or disorder is fibrosis. In some cases, the disease or disorder is duchenne muscular dystrophy. In some cases, the disease or disorder is systemic sclerosis. In some cases, the disease or disorder is selected from a brain disease, vascular disease, liver disease, muscle disease, pancreas disease, adipose tissue associated disease, and inflammation associated disease.

In some cases, a compound or salt of for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), is administered to a subject in need thereof to inhibit CD38 and thereby raise NAD⁺ levels. In some cases, raising NAD+ levels may be useful in treating diseases or conditions indicated to benefit from NAD+including mitochondrial-related diseases or disorders.

In some embodiments, a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), is administered to a subject in need thereof for the treatment and/or prevention of neurodegenerative disease, type I diabetes, insulin resistance, Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra. Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, cystic fibrosis, pancreatitis, idiopathic pulmonary fibrosis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia-telangiectasia, rheumatoid arthritis, lupus, alcohol intolerance, hyperphosphatemia, acute lung injury, and ARDS.

In some cases, a pharmaceutical composition comprising a compound or salt of for a compound or salt of Formula (I), Formula (IA), Formula (IB), Formula (IC), Formula (ID), Formula (II), Formula (IIA), Formula (IIB), Formula (IIC), Formula (IID), Formula (IIE), Formula (IIF), or Formula (IIG), and a pharmaceutically acceptable excipient is adminstered to a subject in need thereof for the treatment of a disease or disorder described herein.

In some embodiments, the compound is selected from

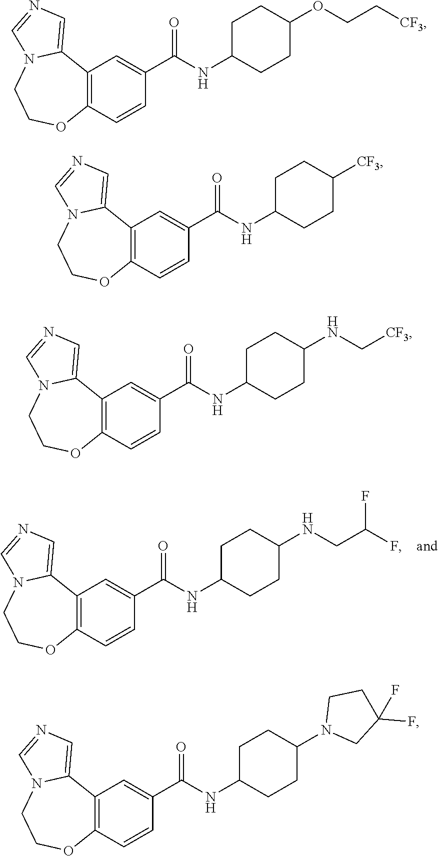

or salt thereof.

### Preparation of Compounds

The compounds of the present disclosure can generally be prepared in a number of ways well known to those skilled in the art of organic synthesis. By way of example, compounds of the present disclosure can be synthesized using the methods described herein, together with synthetic methods known in the art of synthetic organic chemistry, or variations thereof as appreciated by those skilled in the art.

### General Schemes

### Synthetic Schemes for Compounds of Formula (I) and Sub Formulas

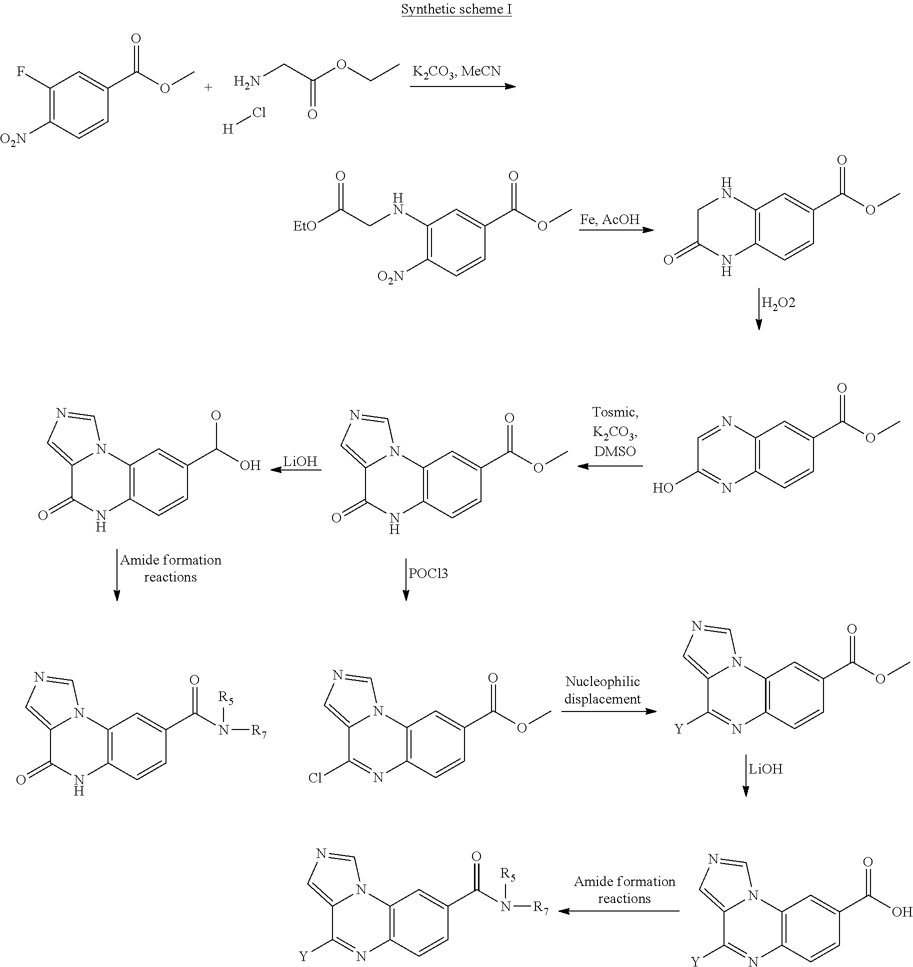

### Synthetic Schemes for Compounds of Formula (II) and Sub Formulas

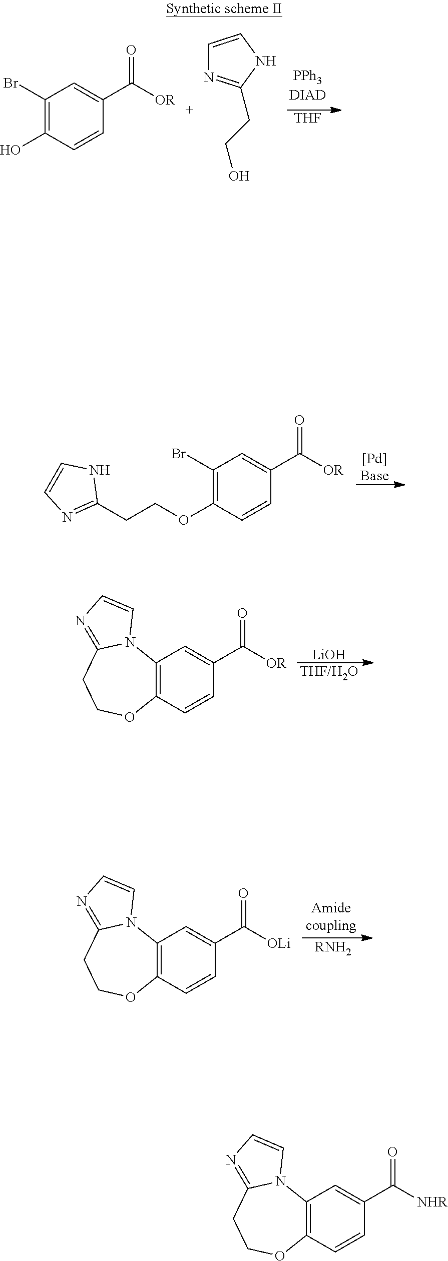

First the corresponding phenol and primary alcohol can undergo a Mitsunobu reaction using triphenylphosphine to generate the desired ether. This can then undergo intramolecular Buckwald coupling using a palladium catalyst and base to generate the desired oxazepine. This can be saponified with lithium hydroxide to generate the corresponding lithium carboxylate salt. This can then be coupled with desired amines using amide coupling conditions to generate the final synthetic compounds.

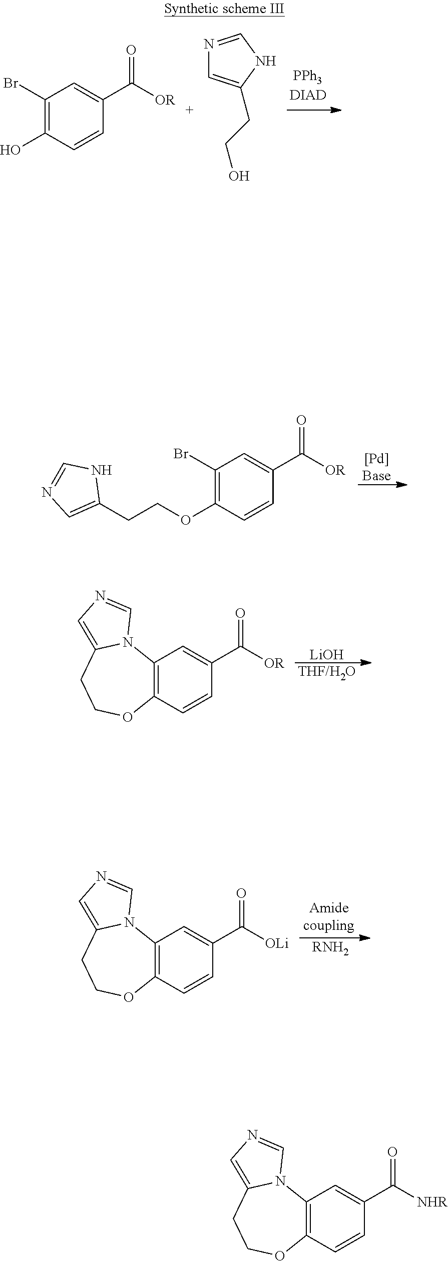

The corresponding phenol and primary alcohol can undergo a Mitsunobu reaction using triphenylphosphine to generate the desired ether. This can then undergo intramolecular Buckwald coupling using a palladium catalyst and base to generate the desired oxazepine. This can be saponified with lithium hydroxide to generate the corresponding lithium carboxylate salt. This can then be coupled with desired amines using amide coupling conditions to generate the final synthetic compounds.

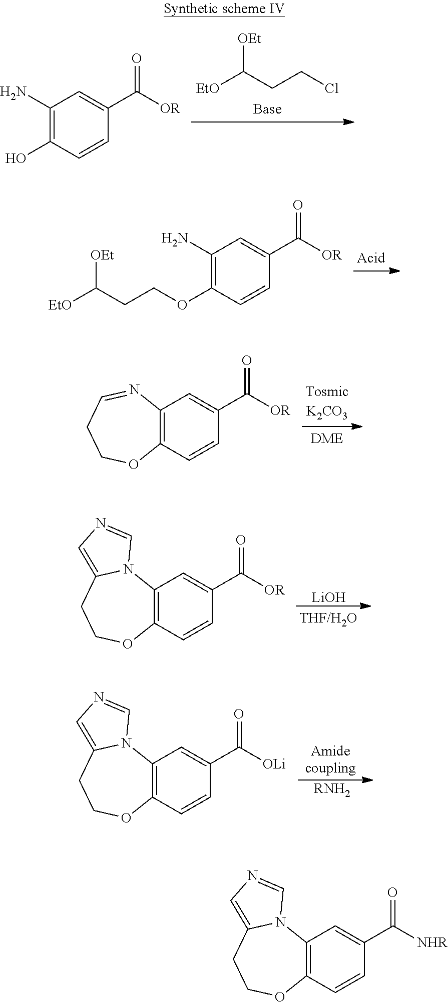

The corresponding phenol and chloro acetyl can be coupled to generate the desired ether. This can be deprotected and cyclise to generate the cyclic imine using an acid. Reaction with tosmic can yield the desired imidazole compound. This can be saponified with lithium hydroxide to generate the corresponding lithium carboxylate salt. This can then be coupled with desire amines using amide coupling conditions to generate the final synthetic compounds.

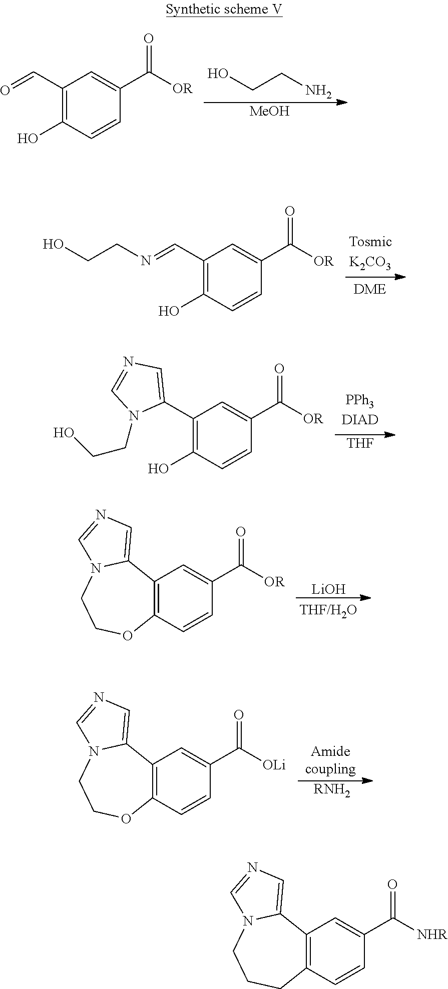

First the corresponding aromatic aldehyde is condensed with ethanolamine to generate the required imine. This is then reacted with Tosmic to produce the substituted imidazole compound. Mitsunobu of the phenol and alcohol tether using triphenylphosphine generates the oxazepine. This is saponified with lithium hydroxide to generate the corresponding lithium carboxylate salt. This can then be coupled with desired amines using amide coupling conditions to generate the final synthetic compounds.

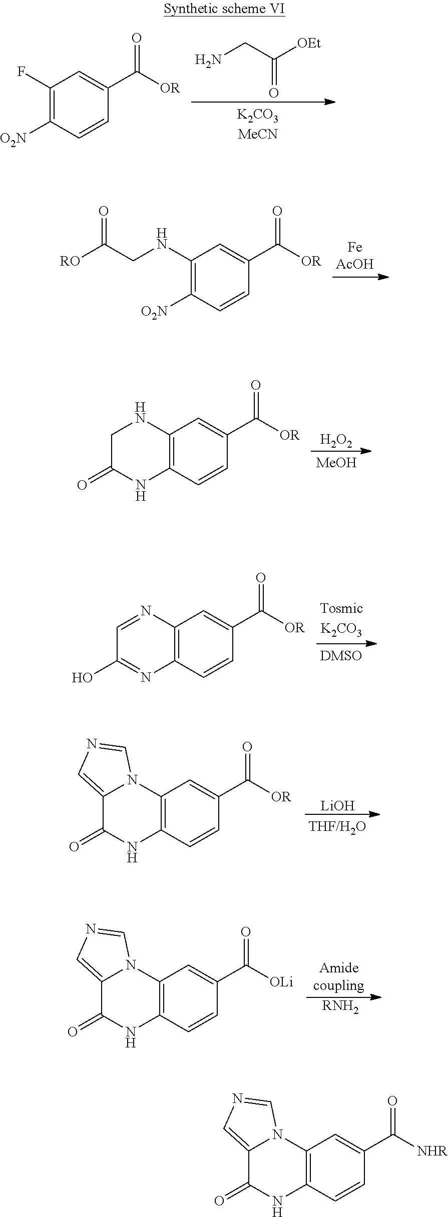

First the corresponding F aromatic ester is reacted with a protected glycine derivative using S_{N}Ar conditions. The isolated nitro compound is then reduced using iron and acetic acid, which catalyses the cyclisation to form the bicyclic species. This is then oxidised to the appropriate quinoxaline using hydrogen peroxide. Reaction with Tosmic generates the desired fused imidazole ring system. This is saponified with lithium hydroxide to generate the corresponding lithium carboxylate salt. This can then be coupled with desired amines using amide coupling conditions to generate the final synthetic compounds.

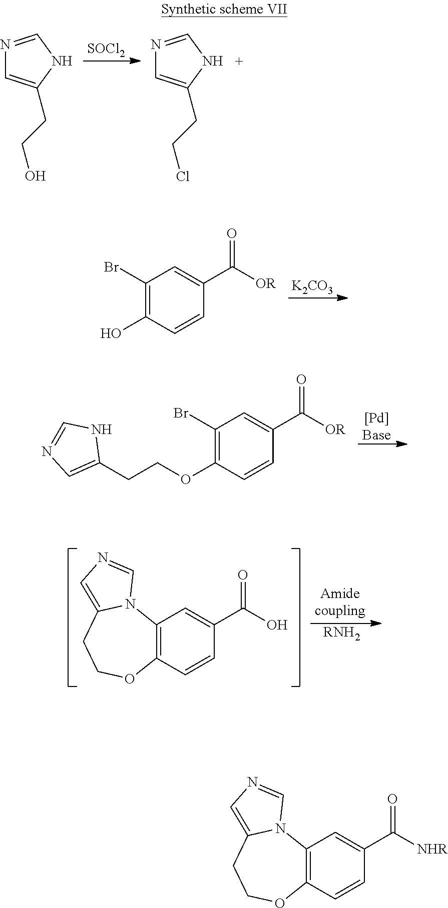

First the corresponding primary alcohol was converted to the corresponding chloride using thionyl chloride. This could be reacted with the substituted phenol with base to generate the desired ether. This can then undergo intramolecular Buchwald coupling using a palladium catalyst and base to generate the desired oxazepane, attempted isolation generated the acid which was immediately coupled with the desired amine using amide coupling conditions to generate the final synthetic compound.

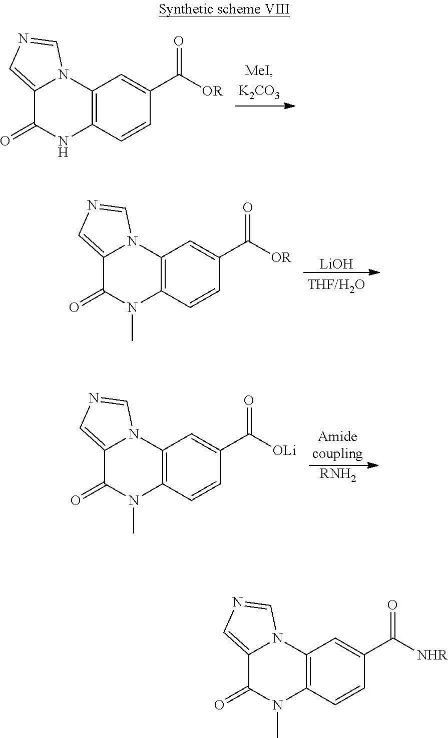

First the corresponding amide is reacted with methyl iodide and potassium carbonate to generate the N-methylated compound, proven by NOESY. This is saponified with lithium hydroxide to generate the corresponding lithium carboxylate salt. This can then be coupled with desired amines using amide coupling conditions to generate the final synthetic compounds.

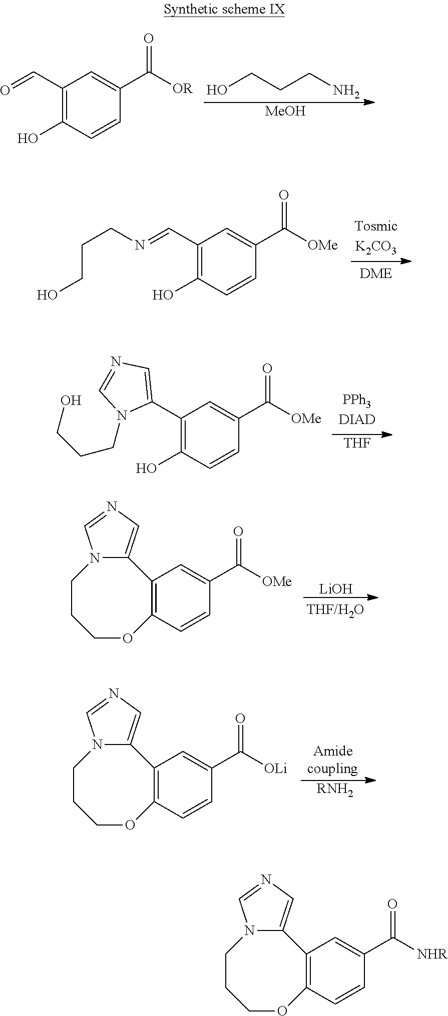

First the corresponding aromatic aldehyde is condensed with 3-amino-1-propanol to generate the required imine. This is then reacted with TosMIC to produce the substituted imidazole compound. Mitsunobu of the phenol and alcohol tether using triphenylphosphine generates the 8 membered heterocycle. This is saponified with lithium hydroxide to generate the corresponding lithium carboxylate salt. This can then be coupled with desired amines using amide coupling conditions to generate the final synthetic compounds.

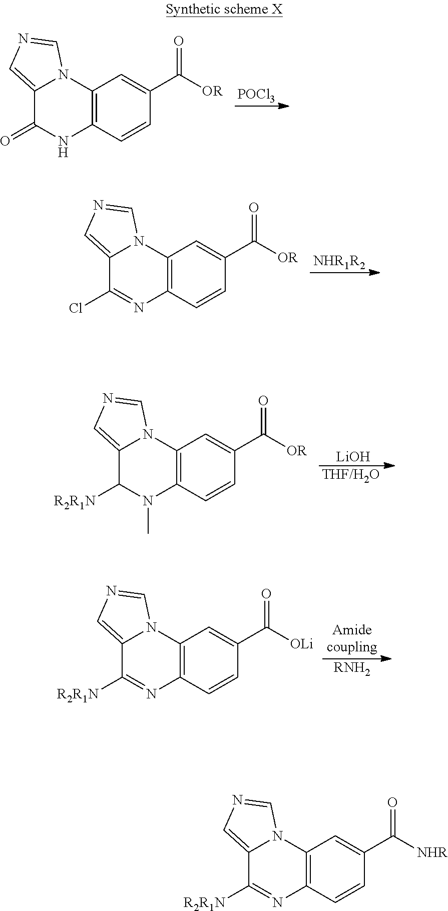

First the corresponding amide is reacted with POCl₃ to generate the chloro compound. This is then reacted with corresponding amines to generate the desired aniline substituted compounds. This is saponified with lithium hydroxide to generate the corresponding lithium carboxylate salt. This can then be coupled with desired amines using amide coupling conditions to generate the final synthetic compounds.

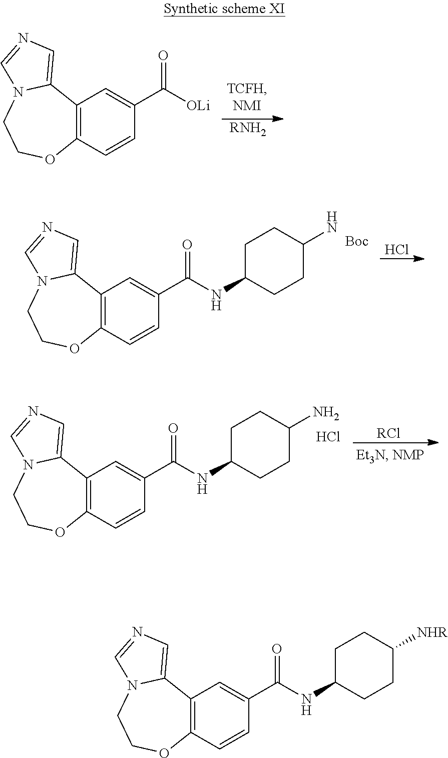

First the corresponding lithium salt is coupled with the protected cyclohexylamine using TCFH and NMI amide coupling conditions to generate the amide. This is then deprotected using HCl in IPA and the amine isolated as the HCl salt. This can be reacted with corresponding sulfonyl/acid chlorides to generate the final synthetic compounds.

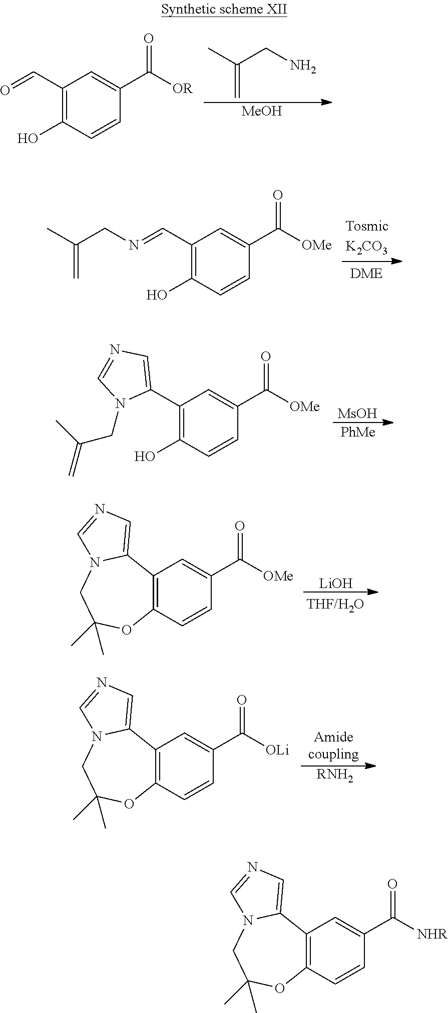

First the corresponding aromatic aldehyde is condensed with 2-methylallylamine to generate the required imine. This is then reacted with TosMIC to produce the substituted imidazole compound. Acid catalysed cyclisation generates the 7 membered heterocycle. This is saponified with lithium hydroxide to generate the corresponding lithium carboxylate salt. This can then be coupled with desired amines using amide coupling conditions to generate the final synthetic compounds.

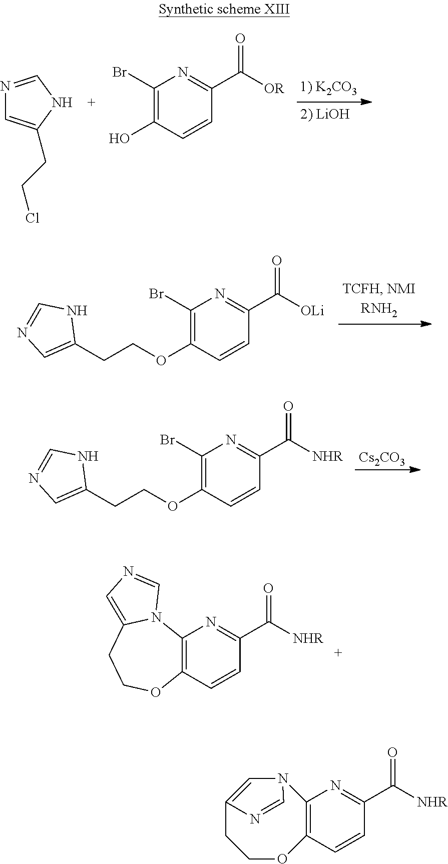

The alkyl chloride could be reacted with the substituted phenol with base to generate the desired ether with the ester hydrolysed immediately after isolation. This can then be coupled with the desired amine using amide coupling conditions to generate the cyclisation precursor. This can then undergo a S_{N}Ar reaction using cesium carbonate to generate the final synthetic compounds.

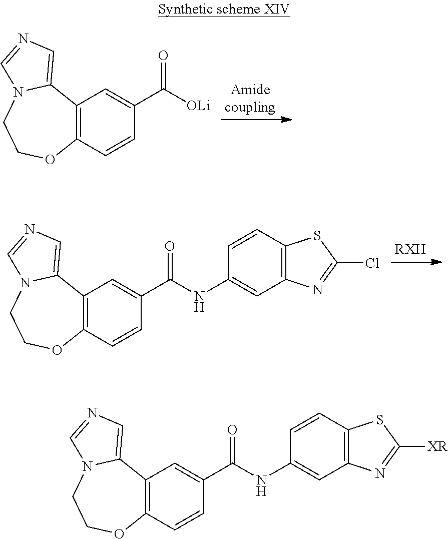

First the corresponding lithium carboxylate salt can be coupled with the aromatic amine to generate the desired amide. This can then be coupled with desired amines/alcohols to generate the final synthetic compounds.

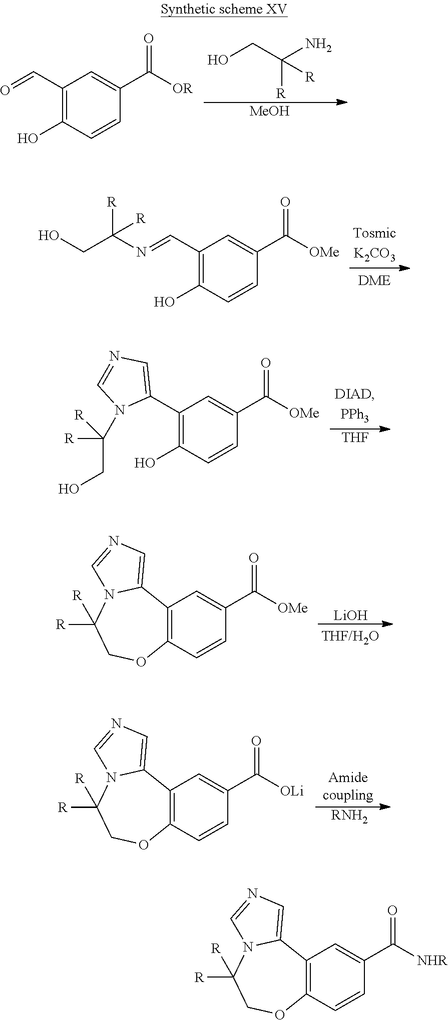

First the corresponding aromatic aldehyde is condensed with 2 substituted amino alcohol to generate the required imine. This is then reacted with TosMIC to produce the substituted imidazole compound. A Mitsunobu reaction with DIAD and triphenylphosphine generates the 7 membered heterocycle. This is saponified with lithium hydroxide to generate the corresponding lithium carboxylate salt. This can then be coupled with desired amines using amide coupling conditions to generate the final synthetic compounds.

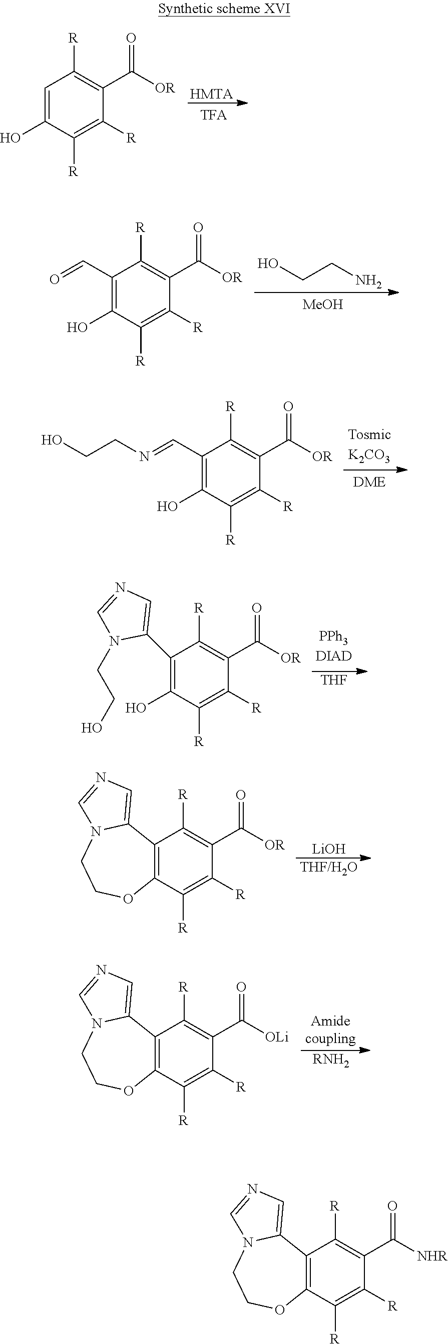

First the corresponding phenol is formylated using HMTA and TFA, hydrolysing with water. The aromatic aldehyde is then condensed with ethanolamine to generate the required imine. This is then reacted with Tosmic to produce the substituted imidazole compound. Mitsunobu of the phenol and alcohol tether using triphenylphosphine generates the oxazepine. This is saponified with lithium hydroxide to generate the corresponding lithium carboxylate salt. This can then be coupled with desired amines using amide coupling conditions to generate the final synthetic compounds.

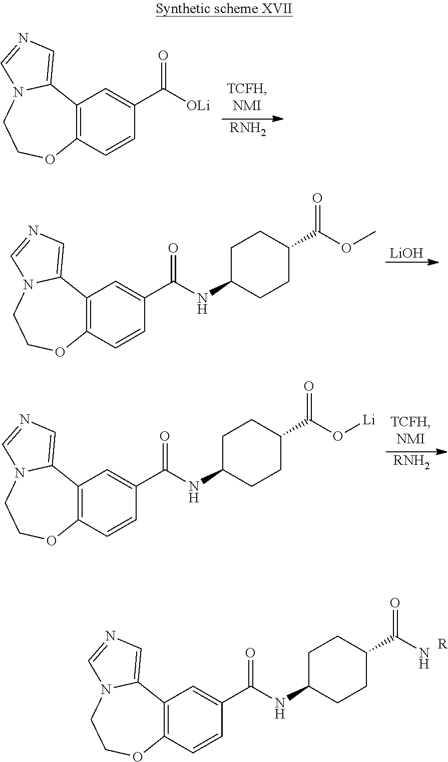

First the corresponding lithium salt is coupled with the functionalised trans-cyclohexylamine using TCFH and NMI amide coupling conditions to generate the amide. The ester is then saponified with lithium hydroxide to generate the lithium salt. This can be reacted with corresponding amines using amide coupling conditions to generate the final synthetic compounds.

### General Procedures

### General Procedure A

Corresponding amine (1.0 eq.) and aldehyde (1.0 eq.) were dissolved in methanol (0.2-1.0 mmol) and stirred at 40° C. for 1-72 h. The reaction mixture was concentrated under reduced pressure to afford the desired product which was used directly in the next step.

### General Procedure B

To a suspension of corresponding imine (1.0 eq.) and potassium carbonate (2.2 eq.) in DME and methanol (˜5:3 ratio, 0.1-1.0 mmol) was added 4-toluenesulfonylmethylisocyanide (1.2 eq.) portionwise and stirred until reaction had reached completion, 18-216 h. The reaction was directly loaded onto an appropriately sized SCX-2 cartridge. The cartridge was washed with methanol (2 CV), then the compound was eluted with 2 M ammonia in methanol (2 CV). The eluent was concentrated to dryness under reduced pressure, with further purification by normal phase chromatography over silica or reverse phase column chromatography over C18 where necessary (in some cases trituration from DMSO/water was also possible) to afford the desired product.

### General Procedure C

To a solution of corresponding phenol (1.0 eq.) and triphenylphosphine (1.2 eq.) in THF (0.05-0.2 mmol) was added diisopropyl azodicarboxylate (1.5 eq.) and stirred until reaction had reached completion, 16-48 h. Additional reagents, (0.5 eq.) and (0.6 eq.) respectively, were added if reaction was observed to have stalled after 18 h. The reaction was directly loaded onto an appropriately sized SCX-2 cartridge. The cartridge was washed with methanol (2 CV), then the compound was eluted with 2 M ammonia in methanol (2 CV). The eluent was concentrated to dryness under reduced pressure, with further purification by normal phase chromatography over silica and/or reverse phase column chromatography over C18 where necessary (in some cases trituration from DMSO/water was also possible) to afford the desired product.

### General Procedure D

A solution of lithium hydroxide monohydrate (1.0-1.05 eq.) in water (0.5-2 mL) was added to a suspension of corresponding ester (1.0 eq.) in tetrahydrofuran (2-6 mL), sometimes with acetonitrile (0.5 mL) as a co-solvent or replacement for THF, and stirred at rt until the reaction had reached completion, 1-120 h. Where reaction had stalled further lithium hydroxide monohydrate (0.5-1.0 eq.) was added. The reaction mixture was then concentrated under reduced pressure to afford the desired product which was used directly in further transformations with no further purification.

### General Procedure E

1-Methylimidazole (4.5 eq.) was added to a suspension of lithium carboxylate salt (1 eq.), corresponding amine or amine hydrochloride (1.1 eq) and chloro-N,N,N′,N′-tetramethylformamidinium hexafluorophosphate (1.2-2.2 eq) in MeCN(1-5 ml) and stirred at 25° C. for 16 h. The crude mixture was diluted with water (10 vol.) and if precipitation occurred the solid was filtered and washed with water to yield the desired product. If precipitation did not occur the solution was extracted with ethyl acetate (2×10 vol.). The combined organic layers were washed with brine (15 vol.), dried (Na₂SO₄), filtered and concentrated under reduced pressure to give crude product. This was purified by reverse phase column chromatography over C18 eluting with a gradient of MeCN(0.1% NH₃) (5% to 95%; v/v) in water (0.1% NH₃), with further purification by preparative HPLC if necessary, to afford the desired product.

### General Procedure F

Hexamethylenetetramine (2.0)-4.6 eq.) was added to a suspension of corresponding phenol (1.0 eq.) and trifluoroacetic acid (5.0-7.0 mL) and the mixture heated at 73° C. for 18-68 h. This was cooled to rt and water (6× volume of TFA) was slowly added before either heating at 80° C. for 2 h or stirring at rt for 1 h. This was cooled and the resulting precipitate filtered and dried, with further purification by normal phase chromatography over silica where necessary, to afford the desired product.

### General Procedure G

4-(Dibenzylamino)cyclohexanone (1.0 eq.), corresponding amine hydrochloride (1.0-1.2 eq.) and acetic acid (1.03-1.15 eq.) were dissolved in DCM (5 mL/mmol) and stirred at rt for 4-96 h. Sodium cyanoborohydride (2.0 eq.) was added and stirred for 24 h at rt. This was concentrated under reduced pressure to give crude product. This was purified by reverse phase column chromatography over C18 eluting with a gradient of MeCN(0.1% NH₃) (5% to 95%: v/v) in water (0.1% NH₃) to afford the desired product.

### General Procedure H

Dibenzylated cyclohexylamine derivative (1.0 eq.) and Palladium hydroxide on carbon (10% loading, 0.2 eq.) were suspended in IPA (5-6 mL) and stirred under an atmosphere of H₂ for 18-42 h. This was filtered through celite, washing with IPA and the combined organic fractions were concentrated under reduced pressure to afford the desired product.

### General Procedure I

Sodium hydride (60% disp. in mineral oil, 2.4 eq.) was added to a solution of corresponding alcohol (1.2 eq.) in anhydrous NMP (10 mL) under N₂ and stirred at rt for 30 minutes. 1-Fluoro-4-nitrobenzene (1 eq.) was added and stirred for 15-60 mins. The reaction mixture was quenched with H₂O and purified directly by column chromatography over C18 eluting with a gradient of MeCN(0.1% NH₃) (5% to 95%: v/v) in water (0.1% NH₃) to afford the desired product.

### General Procedure J

Corresponding substituted nitro benzene (1.0 eq.) and 5% rhodium on alumina (10 mol %) were dissolved in IPA (12 mL) in an ASynt autoclave and the resultant mixture was stirred at room temperature. The apparatus was then pressurised with H₂ to ca. 10 bar and heated to 80° C. for 18 h. The vessel was allowed to cool then depressurised. The reaction mixture was filtered through Celite which was washed with IPA, DCM, MeOH and MeCN. The combined filtrates were concentrated under reduced pressure to afford the desired product.

### General Procedure K

Corresponding substituted nitro benzene (1.0 eq.) and Nishimura's catalyst (15 mol %) were suspended in IPA (˜5.5 mL/mmol) and stirred under an atmosphere of H₂ for 22 h. This was filtered through celite, washing with IPA, DCM, MeOH and MeCN and the combined organic fractions were concentrated under reduced pressure to afford the desired product.

### General Procedure L

Corresponding alcohol (2.05 eq.), N,N-diisopropylethylamine (3.05 eq.), cesium fluoride (1 eq.) and 2-chloro-5-nitropyrimidine (1 eq.) were suspended in DMSO (6 mL) and the resultant mixture was stirred at rt for 0.5-16 h. This was diluted with H₂O and purified directly by column chromatography over C18 eluting with a gradient of MeCN(0.1% NH₃) (5% to 95%; v/v) in water (0.1% NH₃) to afford the desired product.

### General Procedure M

Corresponding nitro compound (1 eq.) and Palladium on activated Carbon (10% loading, paste, 10 mol %) were suspended in IPA (4-8 mL) and stirred under an atmosphere of H₂ for 1-41 h. This was filtered through celite, washing with IPA, DCM and MeOH and the combined organic fractions were concentrated under reduced pressure to afford the desired product.

### EXAMPLES

The following examples are offered to illustrate, but not to limit the claimed invention. It will be recognized that these preparation methods are illustrative and not limiting. Using the teaching provided herein, numerous other methods of producing the compounds described herein will be available to one of skill in the art.

### Example 1: General Analytical Methods

¹H, ¹³C and ¹⁹F NMR analyses were conducted on a JEOL ECZ400s 400 MHz NMR spectrometer using deuterated dimethyl sulfoxide, deuterated acetone, deuterated acetonitrile, deuterated water, or deuterated chloroform as solvent. The shift (δ) of each signal was measured in parts per million (ppm) relative the residual solvent peak, and the multiplicity reported together with the associated coupling constant (J), where applicable.

### Example 2: Waters Acquity UPLC-MS Analysis Methodology

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da: positive and negative modes simultaneously), and ELS detection was achieved using an Acquity UPLC ELS Detector.

Samples were prepared by dissolution (with or without sonication) into 1 mL of 50% (v/v) MeCN in water. The resulting solutions were then filtered through a 0.2 μm syringe filter before submitting for analysis. All the solvents, including 36% ammonia solution, were purchased as the HPLC grade.

Specific differences between methods are as follows:

### Basic 2 Min

0.1% ammonia in water [Eluent A]: 0.1% ammonia in MeCN [Eluent B]: flow rate 0.8 mL/min: column oven 50 C: sample manager 20 C: injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Basic 4 Min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Basic 6 Min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.30 | 95 | 5 |
| 6.00 | 95 | 5 |
| 6.10 | 95 | 5 |
| 7.00 | 95 | 5 |

### Acidic 2 Min

0.1% v/v Formic acid in 10 mM ammonium formate [Eluent A]; 0.1% v/v Formic acid in MeCN [Eluent B]; flow rate 0.8 mL/min; column oven 50° C.; sample manager 20° C.; injection volume 2 μL and 1.5 minutes equilibration time between samples, on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Chiral Prep HPLC

### Purification Method A (P.M. A)

| Columns Details | Chiralpak IA (21 mm × 250 mm, 5 μm | |
|---|---|---|
| Column Temperature | 40° | C. |
| Flow Rate | 50 | mL/min |
| BPR | 100 | BarG |
| Detector Wavelength | 247 | nm |

| | |
|---|---|
| Injection Volume | 400 μL (2.6 mg) |
| Isocratic Conditions | 45:55 MeOH:CO₂ |

### Purification Method B (P.M. B)

| Columns Details | Chiralpak IA (21 mm × 250 mm, 5 μm | |
|---|---|---|
| Column Temperature | 40° | C. |
| Flow Rate | 50 | mL/min |
| BPR | 100 | BarG |
| Detector Wavelength | 246 | nm |

| | |
|---|---|
| Injection Volume | 1300 μL (8.1 mg) |
| Isocratic Conditions | 50:50 MeOH:CO₂ (0.2% v/v NH₃) |

### Example 3: Synthesis of Compounds

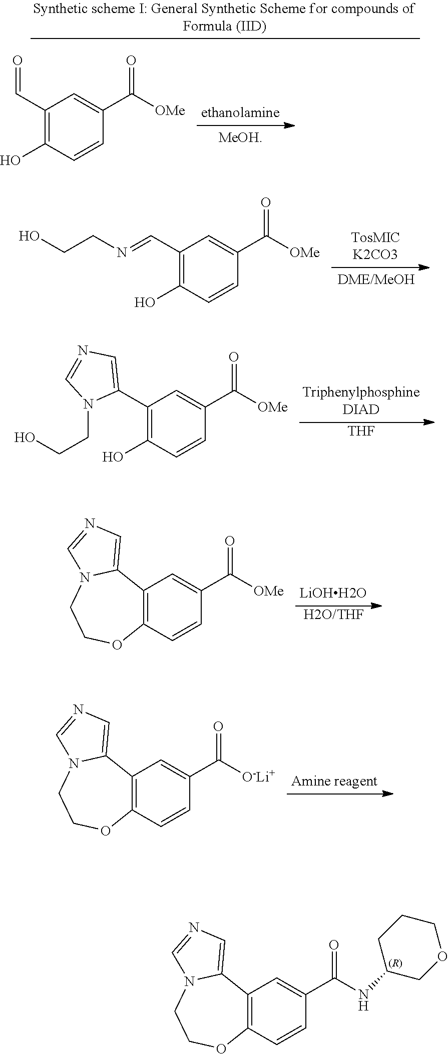

### Synthesis of Methyl 4-hydroxy-3-(((2-hydroxyethyl)imino)methyl)benzoate 1

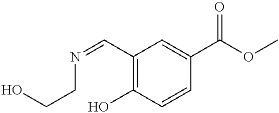 [SMILES: COC(=O)c1ccc(O)c(/C=N\CCO)c1]

To a stirred solution of ethanolamine (0.28 mL, 4.63 mmol) in methanol (5 mL) was added methyl 3-formyl-4-hydroxy benzoate (834 mg, 4.63 mmol) as a solution in methanol (5 mL), the reaction mixture was heated to 40° C. and stirred for 16 h. The reaction mixture was concentrated under reduced pressure to afford the desired product methyl 4-hydroxy-3-(((2-hydroxyethyl)imino)methyl)benzoate 1 (1.03 g, 4.38 mmol, 95% yield) as a yellow solid. ¹H NMR (400 MHz, DMSO-D6) δ 8.63 (s, 1H), 8.07 (d, J=2.4 Hz, 1H), 7.82 (dd, J=9.0, 2.4 Hz, 1H), 6.76 (d, J=8.9 Hz, 1H), 4.93 (t, J=4.5 Hz, 1H), 3.79 (s, 3H), 3.69-3.62 (m, 4H). phenolic proton not observed. UPLC-MS (Basic 2 min): rt=0.68 min, m/z (ES+) 224.1 [M+H]⁺.

### Methyl 4-hydroxy-3-(1-(2-hydroxyethyl)-1H-imidazol-5-yl)benzoate 2

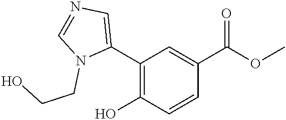 [SMILES: COC(=O)c1ccc(O)c(-c2cncn2CCO)c1]

To a suspension of methyl 4-hydroxy-3-(((2-hydroxyethyl)imino)methyl)benzoate 1 (1.03 g, 4.38 mmol) in dimethoxyethane (12 mL) and methanol (3 mL) was added 4-toluenesulfonylmethylisocyanide (1.03 g, 5.26 mmol) and potassium carbonate (1.33 g, 9.64 mmol), the reaction mixture was stirred under a nitrogen atmosphere at rt for 9 days. The reaction was directly loaded onto an SCX-2 cartridge (10 g). The cartridge was washed with methanol (2 CV), then the compound was eluted with 2 M ammonia in methanol (2 CV). The eluent was concentrated to dryness under reduced pressure to afford the desired product methyl 4-hydroxy-3-(1-(2-hydroxyethyl)-1H-imidazol-5-yl)benzoate 2 (987 mg, 3.65 mmol, 83% yield) as an off-white solid. ¹H NMR (400 MHz, DMSO-D6) δ 7.86 (dd, J=8.5, 2.3 Hz, 1H), 7.74 (d, J=2.2 Hz, 1H), 7.70 (d, J=1.1 Hz, 1H), 7.01 (d, J=8.6 Hz, 1H), 6.85 (d, J=1.1 Hz, 1H), 3.88 (t, J=5.8 Hz, 2H), 3.79 (s, 3H), 3.44 (t, J=5.8 Hz, 2H). UPLC-MS (Basic 2 min): rt=0.61 min, m/z (ES+) 263.0 [M+H]⁺.

### Methyl 5,6-dihydrobenzo[f]imidazo[1,5-d][1,4]oxazepine-10-carboxylate 3

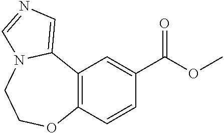 [SMILES: COC(=O)c1ccc2c(c1)-c1cncn1CCO2]

To a solution of methyl 4-hydroxy-3-(1-(2-hydroxyethyl)-1H-imidazol-5-yl)benzoate 2 (150 mg, 0.572 mmol) and triphenylphosphine (180 mg, 0.686 mmol) in THF (4.65 mL) at 0° C. was added diisopropyl azodicarboxylate (0.17 mL, 0.875 mmol), the reaction mixture was allowed to warm to rt and was stirred for 16 h. The reaction was directly loaded onto an SCX-2 cartridge (1 g). The cartridge was washed with methanol (2 CV), then the compound was eluted with 2 M ammonia in methanol (2 CV). The eluent was concentrated to dryness under reduced pressure to afford the desired product methyl 5,6-dihydrobenzo[f]imidazo[1,5-d][1,4]oxazepine-10-carboxylate 3 (134 mg, 0.521 mmol, 91% yield) as an off-white solid. ¹H NMR (400 MHz, DMSO-D6) δ 8.34 (d, J=2.2 Hz, 1H), 7.79 (d, J=1.1 Hz, 1H), 7.73 (dd, J=8.6, 2.2 Hz, 1H), 7.55 (d, J=1.1 Hz, 1H), 7.10 (d, J=8.6 Hz, 1H), 4.53-4.44 (m, 4H), 3.85 (s, 3H). UPLC-MS (Basic 2 min): rt=0.89 min, m/z (ES+) 245.2 [M+H]⁺.

### Lithium 5,6-dihydrobenzo[f]imidazo[1,5-d][1,4]oxazepine-10-carboxylate 4

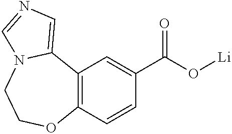

To a solution of methyl 5,6-dihydrobenzo[f]imidazo[1,5-d][1,4]oxazepine-10-carboxylate 3 (133 mg, 0.517 mmol) in THF (2 mL), water (1 mL) and MeCN(0.5 mL) was added lithium hydroxide monohydrate (23 mg, 0.543 mmol), the reaction mixture was stirred at rt for 22 h then concentrated under reduced pressure to afford the desired product lithium 5,6-dihydrobenzo[f]imidazo[1,5-d][1,4]oxazepine-10-carboxylate 4 (122 mg, 0.517 mmol, 99% yield) as a pale grey solid. ¹H NMR (400 MHz, DMSO-D6) δ 8.24 (d, J=2.0 Hz, 1H), 7.72 (d, J=1.1 Hz, 1H), 7.64 (dd, J=8.3, 1.9 Hz, 1H), 7.38 (d, J=1.1 Hz, 1H), 6.85 (d, J=8.3 Hz, 1H), 4.46-4.41 (m, 2H), 4.40-4.34 (m, 2H).

### Methyl 3-((2-ethoxy-2-oxoethyl)amino)-4-nitrobenzoate 5

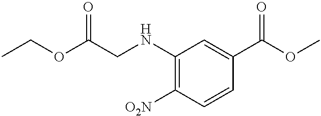 [SMILES: CCOC(=O)CNc1cc(C(=O)OC)ccc1[N+](=O)[O-]]

A suspension of methyl 3-fluoro-4-nitrobenzoate (1.92 g, 9.63 mmol), glycine ethyl ester hydrochloride (1.61 g, 11.6 mmol) and potassium carbonate (3.19 g, 23.1 mmol) in MeCN(49 mL) was heated at 70° C. for 24 h. Water (100 mL) was added and the mixture was extracted with EtOAc (3×100 mL). The organic extracts were combined and washed with water (40 mL) then brine (40 mL), dried (Na₂SO₄) and evaporated to afford the desired product methyl 3-((2-ethoxy-2-oxoethyl)amino)-4-nitrobenzoate 5 (2.61 g, 8.32 mmol, 86% yield) as an orange solid. ¹H NMR (400 MHz, DMSO-D6) δ 8.41 (t, J=5.8 Hz, 1H), 8.21 (d, J=8.8 Hz, 1H), 7.37 (d, J=1.7 Hz, 1H), 7.22 (dd, J=8.8, 1.7 Hz, 1H), 4.33 (d, J=5.8 Hz, 2H), 4.19 (q, J=7.1 Hz, 2H), 3.87 (s, 3H), 1.23 (t, J=7.1 Hz, 3H). UPLC-MS (Basic 2 min): rt=1.08 min, m/z (ES+) not observed.

### Methyl 2-oxo-1,2,3,4-tetrahydroquinoxaline-6-carboxylate 6

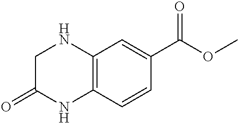

A suspension of methyl 3-((2-ethoxy-2-oxoethyl)amino)-4-nitrobenzoate 5 (2.52 g, 8.57 mmol) and iron (1.72 g, 30.9 mmol) in acetic acid (34 mL) and water (5 mL) was heated at 70° C. for 2 h. The mixture was cooled to 0° C., water (6 mL) was added and the mixture was stirred for 30 min. The solid was collected by filtration, washed with water (60 mL), re-slurried with water (20 mL), collected and dried under vacuum (45° C.) to afford the desired product methyl 2-oxo-1,2,3,4-tetrahydroquinoxaline-6-carboxylate 6 (1.31 g, 6.35 mmol, 74% yield) as a purple solid. ¹H NMR (400 MHz, DMSO-D6) δ 10.58 (s, 1H), 7.28 (d, J=1.9 Hz, 1H), 7.23 (dd, J=8.1, 1.8 Hz, 1H), 6.79 (d, J=8.1 Hz, 1H), 6.25 (s, 1H), 3.81-3.75 (m, 5H). UPLC-MS (Basic 2 min): rt=0.73 min, m/z (ES+) 207.2 [M+H]⁺.

### Methyl 2-hydroxyquinoxaline-6-carboxylate 7

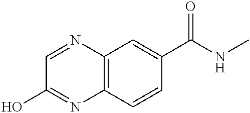

A suspension of methyl 2-oxo-1,2,3,4-tetrahydroquinoxaline-6-carboxylate 6 (106 mg, 0.494 mmol) and hydrogen peroxide, 35 wt. % in water (200 μL, 2.00 mmol) in methanol (2 mL) was stirred for 48 h. A solution of sodium sulfite (188 mg, 1.49 mmol) in water (3 mL) was added and the precipitate was collected, washed with water and dried to afford the desired product methyl 2-hydroxyquinoxaline-6-carboxylate 7 (54 mg, 0.264 mmol, 54% yield) as an orange-brown solid. ¹H NMR (400 MHz, DMSO-D6) δ 12.77-12.48 (m, 1H), 8.35-7.89 (m, 3H), 7.42-7.09 (m, 1H), 3.84 (s, 3H). UPLC-MS (Acidic 2 min): rt=0.77 min, m/z (ES+) 205.1 [M+H]⁺.

### Methyl 4-oxo-4,5-dihydroimidazo[1,5-a]quinoxaline-8-carboxylate 8

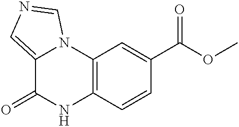 [SMILES: COC(=O)c1ccc2[nH]c(=O)c3cncn3c2c1]

Aqueous 4M potassium carbonate (1.4 mL, 5.49 mmol) was added to a suspension of methyl 2-hydroxyquinoxaline-6-carboxylate 7 (507 mg, 2.38 mmol) and 4-toluenesulfonylmethylisocyanide (698 mg, 3.58 mmol) in DMSO (8 mL) and the mixture was stirred for 3 days. Water (3 mL) was added and the precipitate was collected, washed with water and dried. The solid was stirred in DCM (10 mL) for 1 h. The solid was collected, washed with DCM (5 mL) then MTBE (5 mL) and dried to afford the desired product methyl 4-oxo-4,5-dihydroimidazo[1,5-a]quinoxaline-8-carboxylate 8 (454 mg, 1.87 mmol, 78% yield) as a pale orange solid. ¹H NMR (400 MHz, DMSO-D6) δ 11.71 (s, 1H), 9.26 (d, J=0.9 Hz, 1H), 8.74 (d, J=1.7 Hz, 1H), 7.95 (dd, J=8.5, 1.8 Hz, 1H), 7.90 (d, J=0.8 Hz, 1H), 7.39 (d, J=8.6 Hz, 1H), 3.90 (s, 3H). UPLC-MS (Basic 2 min): rt=0.73 min, m/z (ES+) 244.1 [M+H]⁺.

### Lithium 4-oxo-4,5-dihydroimidazo[1,5-a]quinoxaline-8-carboxylate 9

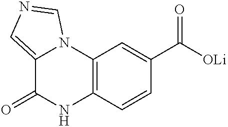

A mixture of methyl 4-oxo-4,5-dihydroimidazo[1,5-a]quinoxaline-8-carboxylate 8 (47 mg, 0.186 mmol) in THF (0.40 mL) and lithium hydroxide (16 mg, 0.371 mmol) in water (0.40 mL) was stirred at 20° C. for 4 days. The solvent was concentrated under reduced pressure to afford the desired product lithium 4-oxo-4,5-dihydroimidazo[1,5-a]quinoxaline-8-carboxylate 9 (44 mg, 0.187 mmol, 100% yield) as a fawn solid. ¹H NMR (400 MHz, DMSO-D6) δ 8.90 (s, 1H), 8.45 (d, J=1.6 Hz, 1H), 7.79 (dd, J=8.3, 1.6 Hz, 1H), 7.66 (s, 1H), 7.11 (dd, J=14.0, 7.9 Hz, 1H). NH signal not observed. UPLC-MS (Basic 2 min): rt=0.18 min, m/z (ES+) 230.2 [M+H]⁺.

### Methyl 4-(2-(1H-imidazol-2-yl)ethoxy)-3-bromobenzoate 10

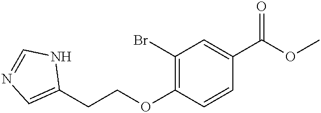 [SMILES: COC(=O)c1ccc(OCCc2cnc[nH]2)c(Br)c1]

To a solution of 2-(1H-imidazol-2-yl) ethanol (121 mg, 1.08 mmol), methyl 3-bromo-4-hydroxy benzoate (250 mg, 1.08 mmol) and triphenylphosphine (341 mg, 1.30 mmol) in THF (10 mL) at 0° C. was added diisopropyl azodicarboxylate (0.33 mL, 1.66 mmol), the reaction mixture was allowed to warm to rt and was stirred for 16 h. The reaction mixture was concentrated under reduced pressure and dissolved in the minimum amount of MeCN and loaded onto an SCX-2 cartridge (2 g). The cartridge was washed with methanol (2 CV), then the compound was eluted with 2 M ammonia in methanol (2 CV). The eluent was concentrated to dryness under reduced pressure and the resulting residue was purified by column chromatography over silica eluting with a gradient of MeOH (0% to 10%; v/v) in DCM to afford the desired product methyl 4-(2-(1H-imidazol-2-yl)ethoxy)-3-bromobenzoate 10 (250 mg, 0.730 mmol, 68% yield) as a white solid. ¹H NMR (400 MHz, DMSO-D6) δ 11.80 (s, 1H), 8.07 (d, J=2.1 Hz, 1H), 7.94 (dd, J=8.7, 2.2 Hz, 1H), 7.27 (d, J=8.7 Hz, 1H), 7.02 (bs, 1H), 6.80 (bs, 1H), 4.46 (t, J=7.0 Hz, 2H), 3.83 (s, 3H), 3.13 (t, J=7.0 Hz, 2H). UPLC-MS (Basic 2 min): rt=0.98 min, m/z (ES+) 326.9 [M+H]⁺.

### Methyl 4,5-dihydrobenzo[b]imidazo[1,2-d][1,4]oxazepine-9-carboxylate 11

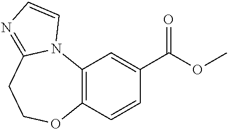 [SMILES: COC(=O)c1ccc2c(c1)-n1ccnc1CCO2]

A suspension of AdBrettPhos Pd G3 (39 mg, 0.0384 mmol), potassium phosphate tribasic (490 mg, 2.31 mmol) and methyl 4-(2-(1H-imidazol-2-yl)ethoxy)-3-bromobenzoate 10 (250 mg, 0.769 mmol) in 1,4-dioxane (7.5 mL) under a nitrogen atmosphere was stirred at 100° C. for 16 h, a portion of Me4tButylXPhos Pd G3 (65 mg, 0.0769 mmol) was added and the reaction stirred for 2 h. An additional portion of Me4tButylXPhos Pd G3 (65 mg, 0.0769 mmol) was added and the reaction mixture was stirred for 64 h, the reaction mixture was cooled to rt, then filtered through a filter paper and the filter cake washed with methanol. The filtrate was concentrated under reduced pressure, then redissolved in methanol (5.0 mL) and loaded onto an SCX-2 cartridge (10 g). The cartridge was washed with methanol (2 CV), then the compound was eluted with 2 M ammonia in methanol (2 CV). The eluent was concentrated to dryness under reduced pressure and the crude residue was purified by column chromatography over silica eluting with a gradient of MeOH (0% to 10%; v/v) in DCM to afford the desired product methyl 4,5-dihydrobenzo[b]imidazo[1,2-d][1,4]oxazepine-9-carboxylate 11 (42 mg, 0.169 mmol, 22% yield) as an orange oil. ¹H NMR (400 MHz, Chloroform-D3) δ 8.04 (d, J=2.1 Hz, 1H), 7.94 (dd, J=8.4, 2.1 Hz, 1H), 7.29-7.23 (m, 2H), 7.10 (d, J=1.5 Hz, 1H), 4.65 (t, J=6.1 Hz, 2H), 3.92 (s, 3H), 3.15 (t, J=6.1 Hz, 2H). UPLC-MS (Basic 2 min): rt=0.88 min, m/z (ES+) 245.3 [M+H]⁺.

### Lithium 4,5-dihydrobenzo[b]imidazo[1,2-d][1,4]oxazepine-9-carboxylate 12

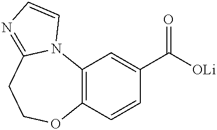

To a suspension of methyl 4,5-dihydrobenzo[b]imidazo[1,2-d][1,4]oxazepine-9-carboxylate 11 (42 mg, 0.172 mmol) in MeCN(1 mL) and water (0.50 mL) was added lithium hydroxide (7.6 mg, 0.181 mmol), the reaction mixture was stirred at rt for 16 h. The reaction mixture was concentrated under reduced pressure to afford the desired product lithium 4,5-dihydrobenzo[b]imidazo[1,2-d][1,4]oxazepine-9-carboxylate 12 (41 mg, 0.169 mmol, 100% yield) as a yellow oil, the crude material was used without further purification. UPLC-MS (Basic 2 min): rt=0.38 min, m/z (ES+) 231.2 [M+H]⁺.

### 5-(2-Chloroethyl)-1H-imidazole 13

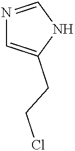

Thionyl chloride (6.2 mL, 86.2 mmol) was added slowly to 2-(1H-imidazol-5-yl) ethanol (250 mg, 2.23 mmol) at 0° C. After the addition was complete, the mixture was warmed to 70° C. for 1 h. The reaction mixture was concentrated under reduced pressure to afford the desired product 5-(2-chloroethyl)-1H-imidazole 13 (300 mg, 1.84 mmol, 82% yield) as a yellow gum. ¹H NMR (400 MHz, DMSO-D6) δ 9.05 (d, J=1.4 Hz, 1H), 7.54 (q, J=1.0 Hz, 1H), 4.85 (s, 1H), 3.93 (t, J=6.5 Hz, 2H), 3.13 (td, J=6.6, 0.9 Hz, 2H). UPLC-MS (Basic 2 min): rt=0.61 min, m/z (ES+) 130.9, 132.9 [M+H]⁺.

### Methyl 4-(2-(1H-imidazol-5-yl)ethoxy)-3-bromobenzoate 14

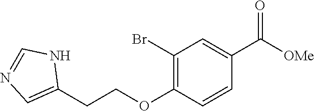 [SMILES: COC(=O)c1ccc(OCCc2cnc[nH]2)c(Br)c1]

5-(2-Chloroethyl)-1H-imidazole 13 (257 mg, 1.97 mmol), methyl 3-bromo-4-hydroxybenzoate (350 mg, 1.51 mmol), sodium iodide (23 mg, 0.151 mmol) and potassium carbonate (628 mg, 4.54 mmol) in DMF (6 mL) were heated to 80° C. and allowed to stir for 18 h. The reaction mixture was loaded directly onto an SCX-2 cartridge (1 g). The cartridge was washed with methanol (2 CV), then the compound was eluted with 2 M ammonia in methanol (2 CV). The eluent was concentrated to dryness under reduced pressure and the crude residue was purified by column chromatography over C18 eluting with a gradient of MeCN(0.1% NH₃) (0% to 90%; v/v) in water (0.1% NH₃) to afford the desired product methyl 4-(2-(1H-imidazol-5-yl)ethoxy)-3-bromobenzoate 14 (170 mg, 0.507 mmol, 33% yield) as a yellow gum. ¹H NMR (400 MHz, Chloroform-D3) δ 8.24 (d, J=2.1 Hz, 1H), 7.96 (dd, J=8.6, 2.1 Hz, 1H), 7.62 (d, J=1.1 Hz, 1H), 6.97 (t, J=0.9 Hz, 1H), 6.91 (d, J=8.7 Hz, 1H), 4.31 (t, J=5.9 Hz, 2H), 3.89 (s, 3H), 3.20 (td, J=6.0, 0.8 Hz, 2H). NH not observed. UPLC-MS (Basic 2 min): rt=0.94 min, m/z (ES+) 327.2 [M+H]⁺.

### Methyl 5-methyl-4-oxo-4,5-dihydroimidazo[1,5-a]quinoxaline-8-carboxylate 15

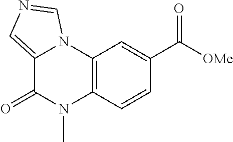 [SMILES: COC(=O)c1ccc2c(c1)n1cncc1c(=O)n2C]

A mixture of methyl 4-oxo-4,5-dihydroimidazo[1,5-a]quinoxaline-8-carboxylate 8 (149 mg, 0.588 mmol), methyl iodide (110 μL, 1.77 mmol) and potassium carbonate (134 mg, 0.970 mmol) in MeCN(1.2 mL) was stirred at 20° C. for 6 days. The mixture was partly evaporated (160 mbar only), the solid was collected, washed with water, then with MeOH and finally dried to afford the desired product methyl 5-methyl-4-oxo-4,5-dihydroimidazo[1,5-a]quinoxaline-8-carboxylate 15 (94 mg, 0.365 mmol, 62% yield) as a pale brown solid. ¹H NMR (400 MHz, DMSO-D6) δ 9.29 (s, 1H), 8.79 (d, J=1.9 Hz, 1H), 8.03 (dd, J=8.8, 1.9 Hz, 1H), 7.92 (s, 1H), 7.66 (d, J=8.8 Hz, 1H), 3.92 (s, 3H), 3.59 (s, 3H). UPLC-MS (Basic 2 min): rt=0.82 min, m/z (ES+) 258.0 [M+H]⁺.

### Lithium 5-methyl-4-oxo-4,5-dihydroimidazo[1,5-a]quinoxaline-8-carboxylate 16

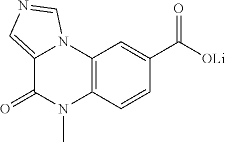

A mixture of methyl 5-methyl-4-oxo-4,5-dihydroimidazo[1,5-a]quinoxaline-8-carboxylate 15 (92 mg, 0.342 mmol) and lithium hydroxide (15 mg, 0.359 mmol) in methanol (0.30 mL), THF (0.60 mL) and water (0.30 mL) was stirred at 20° C. for 5 days. The mixture was evaporated and the solid was freeze-dried to afford the desired product lithium 5-methyl-4-oxo-4,5-dihydroimidazo[1,5-a]quinoxaline-8-carboxylate 16 (113 mg, 0.449 mmol, 100% yield) as a pale brown solid. ¹H NMR (400 MHz, DMSO-D6) δ 8.94 (s, 1H), 8.63 (s, 1H), 8.13-8.06 (m, 1H), 7.94 (s, 1H), 7.61-7.54 (m, 1H), 3.70 (s, 3H). UPLC-MS (Acidic 2 min): rt=0.69 min, m/z (ES+) 244.2 [M+H]⁺.

### Methyl (Z)-4-hydroxy-3-(((3-hydroxypropyl)imino)methyl)benzoate 17

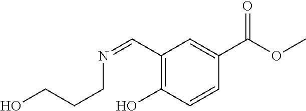 [SMILES: COC(=O)c1ccc(O)c(/C=N\CCCO)c1]

To a stirred suspension of methyl 3-formyl-4-hydroxybenzoate (200 mg, 1.11 mmol) in methanol (3 mL) was added dropwise a solution of 3-amino-1-propanol (0.085 mL, 1.11 mmol) in methanol (3 mL). The resulting yellow solution was heated at 40° C. for 18 h. The reaction mixture was concentrated under reduced pressure to afford the desired product methyl (Z)-4-hydroxy-3-(((3-hydroxypropyl)imino)methyl)benzoate 17 (260 mg, 1.07 mmol, 97% yield) as a yellow solid. ¹H NMR (400 MHz, Chloroform-D3) δ 8.39 (s, 1H), 8.02-7.94 (m, 2H), 6.94 (d, J=8.7 Hz, 1H), 4.82 (s, 2H), 3.89 (s, 3H), 3.84-3.72 (m, 4H), 1.98 (p, J=6.4 Hz, 2H). 2OH not observed. UPLC-MS (Acidic 2 min): rt=0.65 min, m/z (ES+) 238.1 [M+H]⁺.

### Methyl 4-hydroxy-3-(1-(3-hydroxypropyl)-1H-imidazol-5-yl)benzoate 18

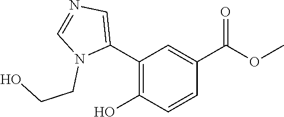 [SMILES: COC(=O)c1ccc(O)c(-c2cncn2CCO)c1]

To a suspension of methyl (Z)-4-hydroxy-3-(((3-hydroxypropyl)imino)methyl)benzoate 17 (260 mg, 1.04 mmol) in dimethoxyethane (5 mL) and methanol (3 mL) was added 4-toluenesulfonylmethylisocyanide (244 mg, 1.25 mmol) and potassium carbonate (317 mg, 2.29 mmol) and the reaction mixture was stirred under a nitrogen atmosphere at room temperature for 20 h. The reaction mixture was loaded directly onto an SCX-2 cartridge (2 g). The cartridge was washed with methanol (2 CV), then the compound was eluted with 2 M ammonia in methanol (2 CV). The eluent was concentrated under reduced pressure to afford the desired product methyl 4-hydroxy-3-(1-(3-hydroxypropyl)-1H-imidazol-5-yl)benzoate 18 (320 mg, 1.01 mmol, 97% yield) as a yellow oil. ¹H NMR (400 MHz, DMSO-D6) δ 7.85 (dd, J=8.5, 2.3 Hz, 1H), 7.74-7.66 (m, 2H), 7.01 (d, J=8.6 Hz, 1H), 6.85 (d, J=1.1 Hz, 1H), 3.92 (dd, J=7.9, 6.6 Hz, 2H), 3.79 (s, 3H), 3.23 (t, J=6.2 Hz, 2H), 1.60 (dq, J=7.9, 6.3 Hz, 2H). 2 OH not observed. UPLC-MS (Basic 2 min): rt=0.62 min, m/z (ES+) 277.2 [M+H]⁺.

### Methyl 6,7-dihydro-5H-benzo[b]imidazo[5,1-d][1,5]oxazocine-11-carboxylate 19

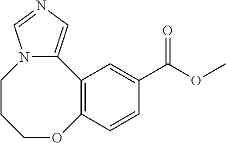 [SMILES: COC(=O)c1ccc2c(c1)-c1cncn1CCCO2]

To a solution of methyl 4-hydroxy-3-(1-(3-hydroxypropyl)-1H-imidazol-5-yl)benzoate 18 (320 mg, 1.01 mmol) and triphenylphosphine (317 mg, 1.21 mmol) in THF (10 mL) at 0° C. was added diisopropyl azodicarboxylate (0.30 mL, 1.54 mmol). The reaction mixture was allowed to stir at room temperature for 18 h. Triphenylphosphine (264 mg, 1.01 mmol) and diisopropyl azodicarboxylate (0.20 mL, 1.01 mmol) were added at room temperature and the resulted reaction mixture was allowed to stir at 40° C. for further 20 h. The reaction mixture was loaded directly onto an SCX-2 cartridge (2 g). The cartridge was washed with methanol (2 CV), then the compound was eluted with 2 M ammonia in methanol (2 CV). The eluent was concentrated under reduced pressure. The residue was purified by column chromatography over C18 eluting with a gradient of MeCN(0.1% NH₃) (0% to 60%; v/v) in water (0.1% NH₃) to afford the desired product methyl 6,7-dihydro-5H-benzo[b]imidazo[5,1-d][1,5]oxazocine-11-carboxylate 19 (60 mg, 0.144 mmol, 14% yield) as a yellow solid. ¹H NMR (400 MHz, Chloroform-D3) δ 8.16-8.02 (m, 1H), 7.95 (dd, J=8.6, 2.2 Hz, 1H), 7.56 (d, J=1.1 Hz, 1H), 7.25 (d, J=1.1 Hz, 1H), 7.08-6.98 (m, 1H), 4.23-4.15 (m, 2H), 4.14-4.06 (m, 2H), 3.90 (s, 3H), 2.04 (p, J=5.6 Hz, 2H). UPLC-MS (Basic 2 min): rt=0.91 min, m/z (ES+) 259.3 [M+H]⁺.

### Lithium 6,7-dihydro-5H-benzo[b]imidazo[5,1-d][1,5]oxazocine-11-carboxylate 20

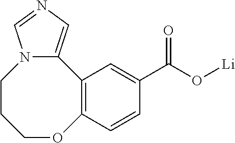

To a solution of methyl 6,7-dihydro-5H-benzo[b]imidazo[5,1-d][1,5]oxazocine-11-carboxylate 19 (60 mg, 0.232 mmol) in THF (3 mL) and water (2 mL) was added lithium hydroxide (9.7 mg, 0.232 mmol) and the reaction mixture was stirred at rt for 18 h. The reaction mixture was concentrated under reduced pressure to afford the desired product lithium 6,7-dihydro-5H-benzo[b]imidazo[5,1-d][1,5]oxazocine-11-carboxylate 20 (70 mg, 0.280 mmol, 100% yield) as a dark colour solid. ¹H NMR (400 MHz, DMSO-D6) δ 7.83-7.79 (m, 2H), 7.71 (d, J=1.2 Hz, 1H), 6.98-6.93 (m, 2H), 4.11 (t, J=5.4 Hz, 2H), 4.06-3.96 (m, 2H), 1.91 (p, J=5.4 Hz, 2H).

### Methyl 4-chloroimidazo[1,5-a]quinoxaline-8-carboxylate 21

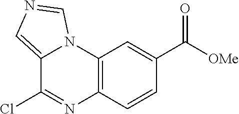 [SMILES: COC(=O)c1ccc2nc(Cl)c3cncn3c2c1]

A solution of methyl 4-oxo-4,5-dihydroimidazo[1,5-a]quinoxaline-8-carboxylate 8 (450 mg, 1.85 mmol) in phosphorous oxychloride (10 mL, 107 mmol) and N,N-dimethylformamide (20 μL, 0.257 mmol) was heated at 105° C. for 18 h. Further phosphorous oxychloride (5.0 mL, 52.2 mmol) and N,N-dimethylformamide (20 μL, 0.257 mmol) were added and heated for 5 days. Further phosphorous oxychloride (6.0 mL, 62.6 mmol) and N,N-dimethylformamide (20 μL, 0.257 mmol) were added and heated for 18 h. Further phosphorous oxychloride (5.0 mL, 52.2 mmol) and N,N-dimethylformamide (0.50 mL) were added and heated for 2 days. The reaction was concentrated under reduced pressure and was quenched with cold water and sat. aq. NaHCO₃ solution and extracted with ethyl acetate. The organic phase was dried (Na₂SO₄), filtered and concentrated under reduced pressure to afford the desired product methyl 4-chloroimidazo[1,5-a]quinoxaline-8-carboxylate 21 (330 mg, 1.05 mmol, 57% yield) as a brown solid. ¹H NMR (400 MHz, DMSO-D6) δ 9.64 (d, J=0.7 Hz, 1H), 8.98 (d, J=1.7 Hz, 1H), 8.12 (dd, J=8.4, 1.8 Hz, 1H), 8.05 (d, J=0.6 Hz, 1H), 8.02-7.87 (m, 1H), 3.96 (s, 3H). UPLC-MS (Basic 2 min): rt=0.97 min, m/z (ES+) 262.2, 264.1 [M+H]⁺.

### tert-Butyl ((1r,4r)-4-(5,6-dihydrobenzo[f]imidazo[1,5-d][1,4]oxazepine-10-carboxamido)cyclohexyl)carbamate 22

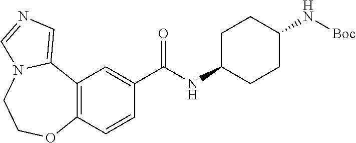 [SMILES: CC(C)(C)OC(=O)N[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and trans-4-(boc-amino)cyclohexylamine according to General Procedure E to afford the desired product tert-butyl ((1r,4r)-4-(5,6-dihydrobenzo[f]imidazo[1,5-d][1,4]oxazepine-10-carboxamido)cyclohexyl)carbamate 22 (367 mg, 0.860 mmol, 60% yield) as an off-white solid. ¹H NMR (400 MHz, DMSO-D6) δ 8.26-8.18 (m, 2H), 7.77 (d, J=1.1 Hz, 1H), 7.65 (dd, J=8.6, 2.2 Hz, 1H), 7.61 (d, J=1.2 Hz, 1H), 7.03 (d, J=8.6 Hz, 1H), 6.76 (d, J=8.0 Hz, 1H), 4.51-4.39 (m, 4H), 3.77-3.65 (m, 1H), 3.26-3.14 (m, 1H), 1.89-1.77 (m, 4H), 1.46-1.17 (m, 13H). UPLC-MS (Basic 2 min): rt=0.99 min, m/z (ES+) 427.3 [M+H]⁺.

### N-((1r,4r)-4-Aminocyclohexyl)-5,6-dihydrobenzo[f]imidazo[1,5-d][1,4]oxazepine-10-carboxamide hydrochloride 23

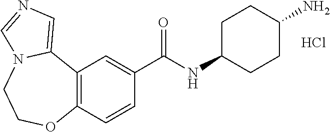 [SMILES: N[C@H]1CC[C@H](NC(=O)c2ccc3c(c2)-c2cncn2CCO3)CC1 ; Cl]

A suspension of tert-butyl ((1r,4r)-4-(5,6-dihydrobenzo[f]imidazo[1,5-d][1,4]oxazepine-10-carboxamido)cyclohexyl)carbamate 22 (367 mg, 0.860 mmol) in IPA (15 mL) was treated dropwise with Hydrogen chloride solution 6 M in IPA (2.4 mL, 12.0 mmol) and the resulting mixture was heated at 55° C. for 18 h. This was then concentrated under reduced pressure, triturated with ether, and filtered to afford the desired product N-((1r,4r)-4-aminocyclohexyl)-5,6-dihydrobenzo[f]imidazo[1,5-d][1,4]oxazepine-10-carboxamide hydrochloride 23 (295 mg, 0.813 mmol, 60% yield) as a white solid. ¹H NMR (400 MHz, DMSO-D6) δ 9.10 (s, 1H), 8.59-8.47 (m, 2H), 8.43 (d, J=2.2 Hz, 1H), 8.19-7.96 (m, 3H), 7.84 (dd, J=8.7, 2.2 Hz, 1H), 7.14 (d, J=8.6 Hz, 1H), 4.73-4.66 (m, 2H), 4.61-4.55 (m, 2H), 3.83-3.70 (m, 1H), 3.06-2.90 (m, 1H), 2.08-1.87 (m, 4H), 1.58-1.40 (m, 4H). UPLC-MS (Basic 4 min): rt=0.94 min, m/z (ES+) 327.1 [M+H]⁺.

### Methyl (E)-4-hydroxy-3-(((2-methylallyl)imino)methyl)benzoate 24

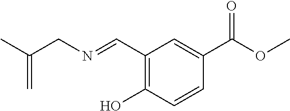 [SMILES: C=C(C)C/N=C/c1cc(C(=O)OC)ccc1O]

2-Methylallylamine (99 mg, 1.39 mmol) and methyl 3-formyl-4-hydroxybenzoate (834 mg, 4.63 mmol) in methanol (5 mL) were heated at 40° C. for 72 h. The reaction mixture was concentrated under reduced pressure to afford the desired product methyl (E)-4-hydroxy-3-(((2-methylallyl)imino)methyl)benzoate 24 (350 mg, 1.05 mmol, 76% yield) as a yellow oil. 1H NMR (400 MHz, DMSO-D6) δ 8.71 (s, 1H), 8.12 (d, J=2.3 Hz, 1H), 7.88 (dd, J=8.8, 2.3 Hz, 1H), 6.89 (d, J=8.8 Hz, 1H), 4.94-4.84 (m, 2H), 4.19 (s, 2H), 3.81 (s, 3H), 1.77 (t, J=1.1 Hz, 3H). OH not observed. UPLC-MS (Basic 2 min): rt=1.06 min, m/z (ES+) 234.1 [M+H]⁺.

### Methyl 4-hydroxy-3-(1-(2-methylallyl)-1H-imidazol-5-yl)benzoate 25

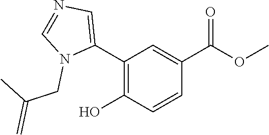 [SMILES: C=C(C)Cn1cncc1-c1cc(C(=O)OC)ccc1O]

To a suspension of methyl (E)-4-hydroxy-3-(((2-methylallyl)imino)methyl)benzoate 24 (350 mg, 1.05 mmol) in dimethoxyethane (5 mL) and methanol (3 mL) was added 4-toluenesulfonylmethylisocyanide (246 mg, 1.26 mmol) and potassium carbonate (319 mg, 2.31 mmol), the reaction mixture was stirred under a nitrogen atmosphere at rt for 68 h. The reaction mixture was loaded directly onto an SCX-2 cartridge (20 g). The cartridge was washed with methanol (2 CV), then the compound was eluted with 2 M ammonia in methanol (2 CV). The eluent was concentrated under reduced pressure to afford the desired product methyl 4-hydroxy-3-(1-(2-methylallyl)-1H-imidazol-5-yl)benzoate 25 (270 mg, 0.992 mmol, 94% yield) as an off-white solid. ¹H NMR (400 MHz, DMSO-D6) δ 7.83 (dd, J=8.5, 2.3 Hz, 1H), 7.72-7.65 (m, 2H), 7.01 (d, J=8.5 Hz, 1H), 6.93 (d, J=1.1 Hz, 1H), 4.72-4.66 (m, 1H), 4.48 (s, 2H), 4.30 (s, 1H), 3.79 (s, 3H), 1.47 (s, 3H). OH not observed. UPLC-MS (Basic 2 min): rt=0.80 min, m z (ES+) 273.0 [M+H]⁺.

### Methyl 6,6-dimethyl-5,6-dihydrobenzo[f]imidazo[1,5-d][1,4]oxazepine-10-carboxylate 26

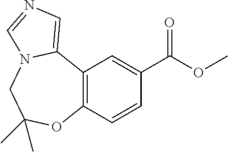

Methanesulfonic acid (66 μL, 1.01 mmol) was added to a suspension of methyl 4-hydroxy-3-(1-(2-methylallyl)-1H-imidazol-5-yl)benzoate 25 (250 mg, 0.918 mmol) in toluene (30 mL) at 80° C. and stirred for 68 h. This was then cooled and concentrated under reduced pressure to give a brown solid. The crude material was purified by column chromatography over C18 eluting with a gradient of MeCN(0.1% NH₃) (5% to 95%; v/v) in water (0.1% NH₃) to afford the desired product methyl 6,6-dimethyl-5,6-dihydrobenzo[f]imidazo[1,5-d][1,4]oxazepine-10-carboxylate 26 (31 mg, 0.102 mmol, 11% yield) as a yellow oil. ¹H NMR (400 MHz, DMSO-D6) δ 8.09 (d, J=2.2 Hz, 1H), 7.85 (dd, J=8.4, 2.2 Hz, 1H), 7.81 (d, J=1.1 Hz, 1H), 7.36 (d, J=1.0 Hz, 1H), 7.12 (d, J=8.4 Hz, 1H), 4.17 (s, 2H), 3.85 (s, 3H), 1.33 (s, 6H). UPLC-MS (Basic 2 min): rt=0.97 min, m/z (ES+) 273.3 [M+H]⁺.

### Lithium 6,6-dimethyl-5,6-dihydrobenzo[f]imidazo[1,5-d][1,4]oxazepine-10-carboxylate 27

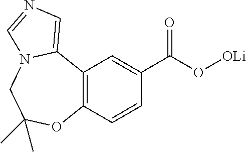

A solution of lithium hydroxide (4.4 mg, 0.104 mmol) in water (0.80 mL) was added to a solution of methyl 6,6-dimethyl-5,6-dihydrobenzo[f]imidazo[1,5-d][1,4]oxazepine-10-carboxylate 26 (30 mg, 0.0992 mmol) in THF (2 mL) and stirred at 25° C. for 18 h. This was concentrated under reduced pressure to afford the desired product lithium 6,6-dimethyl-5,6-dihydrobenzo[f]imidazo[1,5-d][1,4]oxazepine-10-carboxylate 27 (30 mg, 0.0976 mmol, 98% yield) as a yellow solid. ¹H NMR (400 MHz, DMSO-D6) δ 7.92 (d, J=2.0 Hz, 1H), 7.76 (d, J=1.0 Hz, 1H), 7.73 (dd, J=8.2, 2.0 Hz, 1H), 7.14 (d, J=1.0 Hz, 1H), 6.86 (d, J=8.2 Hz, 1H), 3.99 (s, 2H), 1.31 (s, 6H). UPLC-MS (Basic 2 min): rt=0.63 min, m/z (ES+) 259.0 [M+H]⁺.

### Lithium 5-(2-(1H-imidazol-5-yl)ethoxy)-6-bromopicolinate 28

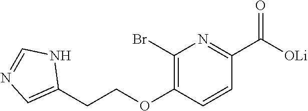

5-(2-Chloroethyl)-1H-imidazole 13 (338 mg, 2.59 mmol), methyl 6-bromo-5-hydroxypyridine-2-carboxylate (150 mg, 0.646 mmol), sodium iodide (9.7 mg, 0.0646 mmol) and potassium carbonate (268 mg, 1.94 mmol) in DMF (6 mL) was heated to 80° C. and allowed to stir for 18 h. Further 5-(2-chloroethyl)-1H-imidazole 13 (178 mg, 1.29 mmol) in DMF (1 mL) and potassium carbonate (268 mg, 1.94 mmol) were added and the reaction heated for a further 48 h. The reaction mixture was purified directly by column chromatography over C18 eluting with a gradient of MeCN(0.1% NH₃) (0% to 50%; v/v) in water (0.1% NH₃) to afford a mixture of hydrolysed acid and methyl ester (195 mg) as a yellow colour gum. This was dissolved in THF (2 mL), water (1 mL) and MeCN(2 mL) and lithium hydroxide (9.8 mg, 0.233 mmol) was added, the reaction mixture was stirred at rt for 3 h, then concentrated under reduced pressure to afford the desired product lithium 5-(2-(1H-imidazol-5-yl)ethoxy)-6-bromopicolinate 28 (185 mg, 0.372 mmol, 58%) as a dark brown colour gum. UPLC-MS (Basic 2 min): rt=0.45 min, m z (ES+) not observed.

### (R)-5-(2-(1H-Imidazol-5-yl)ethoxy)-6-bromo-N-(tetrahydro-2H-pyran-3-yl) picolinamide 29

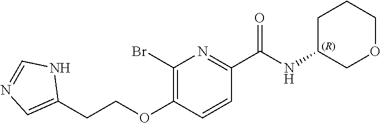

Prepared from lithium 5-(2-(1H-imidazol-5-yl)ethoxy)-6-bromopicolinate 28 and (R)-tetrahydro-2H-pyran-3-amine hydrochloride according to General Procedure E to afford the desired product (R)-5-(2-(1H-imidazol-5-yl)ethoxy)-6-bromo-N-(tetrahydro-2H-pyran-3-yl) picolinamide 29 (55 mg, 0.100 mmol, 27% yield) as a yellow gum. UPLC-MS (Acidic 2 min): rt=0.71 min, m/z (ES+) 395.3, 397.3 [M+H]⁺.

### N-(2-Chlorobenzo[d]thiazol-5-yl)-5,6-dihydrobenzo[f]imidazo[1,5-d][1,4]oxazepine-10-carboxamide 30

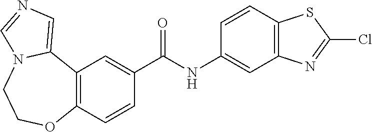 [SMILES: O=C(Nc1ccc2sc(Cl)nc2c1)c1ccc2c(c1)-c1cncn1CCO2]

Prepared from lithium 5,6-dihydrobenzo[f]imidazo[1,5-d][1,4]oxazepine-10-carboxylate 4 and according to General Procedure E to afford the desired product N-(2-chlorobenzo[d]thiazol-5-yl)-5,6-dihydrobenzo[f]imidazo[1,5-d][1,4]oxazepine-10-carboxamide 30 (663 mg, 1.55 mmol, 82% yield) as a beige solid. ¹H NMR (400 MHz, DMSO-D6) δ 10.49 (s, 1H), 8.50 (d, J=2.0 Hz, 1H), 8.43 (d, J=2.3 Hz, 1H), 8.07 (d, J=8.8 Hz, 1H), 7.95 (s, 1H), 7.87 (dd, J=8.9, 2.1 Hz, 1H), 7.82 (dd, J=8.6, 2.2 Hz, 1H), 7.79-7.75 (m, 1H), 7.15 (d, J=8.5 Hz, 1H), 4.56-4.48 (m, 4H). UPLC-MS (Basic 4 min): rt=1.62 min, m/z (ES+) 397.0, 399.0

### Methyl (E)-4-hydroxy-3-(((1-hydroxy-2-methylpropan-2-yl)imino)methyl)benzoate 31

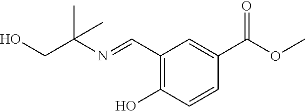 [SMILES: COC(=O)c1ccc(O)c(/C=N/C(C)(C)CO)c1]

2-Amino-2-methyl-1-propanol (0.11 mL, 1.11 mmol) and methyl 3-formyl-4-hydroxy benzoate (200 mg, 1.11 mmol) in methanol (5 mL) were heated to 40° C. for 3 h. The reaction mixture was concentrated under reduced pressure to afford the desired product methyl (E)-4-hydroxy-3-(((1-hydroxy-2-methylpropan-2-yl)imino)methyl)benzoate 31 (275 mg, 1.08 mmol, 98% yield) as a yellow solid. ¹H NMR (400 MHz, DMSO-D6) δ 8.69 (s, 1H), 8.14 (d, J=2.4 Hz, 1H), 7.78 (ddd, J=9.1, 2.4, 0.8 Hz, 1H), 6.65 (d, J=9.0 Hz, 1H), 5.18 (s, 1H), 3.78 (s, 3H), 3.40 (s, 2H), 1.30 (d, J=0.7 Hz, 6H). UPLC-MS (Basic 2 min): rt=0.81 min, m/z (ES+) 252.1 [M+H]⁺.

### Methyl 4-hydroxy-3-(1-(1-hydroxy-2-methylpropan-2-yl)-1H-imidazol-5-yl)benzoate

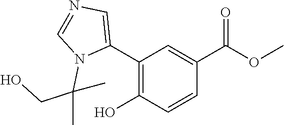 [SMILES: COC(=O)c1ccc(O)c(-c2cncn2C(C)(C)CO)c1]

4-Toluenesulfonylmethylisocyanide (256 mg, 1.31 mmol) was added to a suspension of methyl (E)-4-hydroxy-3-(((1-hydroxy-2-methylpropan-2-yl)imino)methyl)benzoate 31 (275 mg, 1.09 mmol) and potassium carbonate (333 mg, 2.41 mmol) in dimethoxyethane (5 mL) and methanol (3 mL) at 25° C. and stirred for 18 h. The reaction was directly loaded onto an SCX-2 cartridge (10 g). The cartridge was washed with methanol (2 CV), then the compound was eluted with 2 M ammonia in methanol (2 CV). The eluent was concentrated to dryness under reduced pressure to give crude product. This was purified by column chromatography over C18 eluting with a gradient of MeCN(0.1% NH₃) (5% to 40%; v/v) in water (0.1% NH₃) and this was concentrated under reduced pressure. The resulting solid was triturated with MeCN to afford the desired product methyl 4-hydroxy-3-(1-(1-hydroxy-2-methylpropan-2-yl)-1H-imidazol-5-yl)benzoate (245 mg, 0.844 mmol, 77% yield) as a white solid. ¹H NMR (400 MHz, DMSO-D6) δ 7.84 (dd, J=8.5, 2.3 Hz, 1H), 7.76-7.70 (m, 2H), 6.92 (d, J=8.4 Hz, 1H), 6.64 (d, J=1.2 Hz, 1H), 3.78 (s, 3H), 3.42 (s, 2H), 1.30 (s, 6H). 2 OH not observed. UPLC-MS (Basic 2 min): rt=0.74 min, m/z (ES+) 291.1 [M+H]⁺.

### Methyl 5,5-dimethyl-5,6-dihydrobenzo[f]imidazo[1,5-d][1,4]oxazepine-10-carboxylate

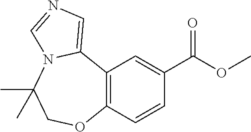

Diisopropyl azodicarboxylate (0.25 mL, 1.27 mmol) was added to a suspension of triphenylphosphine (266 mg, 1.01 mmol) and methyl 4-hydroxy-3-(1-(1-hydroxy-2-methylpropan-2-yl)-1H-imidazol-5-yl)benzoate (245 mg, 0.844 mmol) in THF (13 mL) at 25° C. and stirred for 68 h. The reaction mixture was directly loaded onto an SCX-2 cartridge (10 g). The cartridge was washed with MeOH (2 CV), then the compound was eluted with 2M ammonia in methanol (2 CV). The eluent was concentrated to dryness under reduced pressure to afford the desired product methyl 5,5-dimethyl-5,6-dihydrobenzo[f]imidazo[1,5-d][1,4]oxazepine-10-carboxylate (229 mg, 0.841 mmol, 99% yield) as a white solid. ¹H NMR (400 MHz, DMSO-D6) δ 8.37 (t, J=2.0 Hz, 1H), 8.06 (t, J=1.4 Hz, 1H), 7.77-7.69 (m, 1H), 7.51 (t, J=1.5 Hz, 1H), 7.13 (dd, J=8.5, 1.7 Hz, 1H), 4.28 (s, 2H), 3.85 (s, 3H), 1.59 (s, 6H). UPLC-MS (Basic 2 min): rt=0.97 min, m/z (ES+) 273.3 [M+H]⁺.

### Lithium 5,5-dimethyl-5,6-dihydrobenzo[f]imidazo[1,5-d][1,4]oxazepine-10-carboxylate

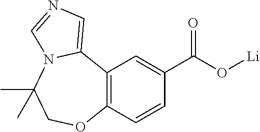

A solution of lithium hydroxide (37 mg, 0.883 mmol) in water (1.5 mL) was added to a solution of methyl 5,5-dimethyl-5,6-dihydrobenzo[f]imidazo[1,5-d][1,4]oxazepine-10-carboxylate (229 mg, 0.841 mmol) in THF (6 mL) at 25° C. and stirred for 48 h. This was concentrated under reduced pressure to afford the desired product lithium 5,5-dimethyl-5,6-dihydrobenzo[f]imidazo[1,5-d][1,4]oxazepine-10-carboxylate (235 mg, 0.845 mmol, 100% yield) as an off-white solid. ¹H NMR (400 MHz, DMSO-D6) δ 8.28 (d, J=1.8 Hz, 1H), 7.99 (d, J=1.1 Hz, 1H), 7.63 (dd, J=8.3, 1.9 Hz, 1H), 7.34 (d, J=1.1 Hz, 1H), 6.86 (d, J=8.3 Hz, 1H), 4.16 (s, 2H), 1.57 (s, 6H). UPLC-MS (Basic 2 min): rt=0.65 min, m/z (ES+) 259.1 [M+H]+

### Methyl 3-bromo-5-formyl-4-hydroxybenzoate

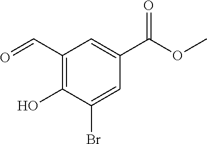

Hexamethylenetetramine (1.40 g, 9.95 mmol) was added to a suspension of methyl 3-bromo-4-hydroxy benzoate (500 mg, 2.16 mmol) and trifluoroacetic acid (5.0 mL, 65.3 mmol) and the mixture heated at 73° C. for 18 h. This was cooled to rt and water (30 mL) was slowly added before heating at 80° C. for 2 h. This was cooled and the resulting precipitate filtered and dried to afford the desired product methyl 3-bromo-5-formyl-4-hydroxybenzoate (570 mg, 1.76 mmol, 81% yield) as an off-white solid. ¹H NMR (400 MHz, DMSO-D6) δ 10.16 (s, 1H), 8.35 (d, J=2.2 Hz, 1H), 8.31 (d, J=2.2 Hz, 1H), 3.86 (s, 3H). UPLC-MS (Basic 2 min): rt=0.69 min, m/z (ES+) not observed.

### Methyl (E)-3-bromo-4-hydroxy-5-(((2-hydroxyethyl)imino)methyl)benzoate

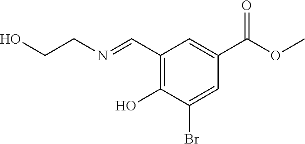 [SMILES: COC(=O)c1cc(Br)c(O)c(/C=N/CCO)c1]

Ethanolamine (0.11 mL, 1.76 mmol) and methyl 3-bromo-5-formyl-4-hydroxy benzoate (570 mg, 1.76 mmol) in methanol (10 mL) were heated to 40° C. for 68 h. The reaction mixture was concentrated under reduced pressure to afford the desired product methyl (E)-3-bromo-4-hydroxy-5-(((2-hydroxyethyl)imino)methyl)benzoate (680 mg, 1.76 mmol, 100% yield) as a yellow solid. ¹H NMR (400 MHz, DMSO-D6) δ 8.68 (s, 1H), 8.07 (d, J=2.3 Hz, 1H), 8.04 (d, J=2.3 Hz, 1H), 5.13 (s, 1H), 3.77 (s, 3H), 3.73-3.62 (m, 5H).

### Methyl 3-bromo-4-hydroxy-5-(1-(2-hydroxyethyl)-1H-imidazol-5-yl)benzoate

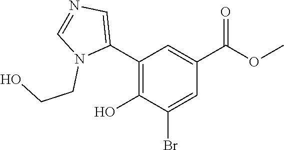 [SMILES: COC(=O)c1cc(Br)c(O)c(-c2cncn2CCO)c1]

4-Toluenesulfonylmethylisocyanide (422 mg, 2.16 mmol) was added to a suspension of methyl (E)-3-bromo-4-hydroxy-5-(((2-hydroxyethyl)imino)methyl)benzoate (680 mg, 1.80 mmol) and potassium carbonate (547 mg, 3.96 mmol) in dimethoxyethane (10 mL) and methanol (6 mL) at 25° C. and stirred for 18 h. This was filtered and concentrated under reduced pressure to yield crude product. This was purified by column chromatography over C18 eluting with a gradient of MeCN(0.1% NH₃) (5% to 25%; v/v) in water (0.1% NH₃) to afford the desired product methyl 3-bromo-4-hydroxy-5-(1-(2-hydroxyethyl)-1H-imidazol-5-yl)benzoate (518 mg, 1.37 mmol, 76% yield) as a white solid. ¹H NMR (400 MHz, DMSO-D6) δ 7.84 (d, J=2.5 Hz, 1H), 7.58 (d, J=1.2 Hz, 1H), 7.43 (d, J=2.5 Hz, 1H), 6.58 (d, J=1.2 Hz, 1H), 5.79 (s, 1H), 3.87 (t, J=5.6 Hz, 2H), 3.66 (s, 3H), 3.57 (t, J=5.6 Hz, 2H). 1 OH not observed. UPLC-MS (Basic 2 min): rt=0.68 min, m/z (ES+) 341.2, 343.2 [M+H]⁺.

### Methyl 8-bromo-5,6-dihydrobenzo[f]imidazo[1,5-d][1,4]oxazepine-10-carboxylate

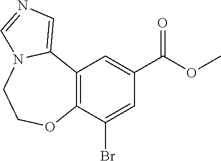 [SMILES: COC(=O)c1cc(Br)c2c(c1)-c1cncn1CCO2]

Diisopropyl azodicarboxylate (0.36 mL, 1.84 mmol) was added to a solution of methyl 3-bromo-4-hydroxy-5-(1-(2-hydroxyethyl)-1H-imidazol-5-yl)benzoate (465 mg, 1.23 mmol) and triphenylphosphine (386 mg, 1.47 mmol) in THF (25 mL) at 0° C. and the reaction stirred at rt for 18 h. Further triphenylphosphine (193 mg, 0.74 mmol) and diisopropyl azodicarboxylate (0.18 mL, 0.92 mmol) were added and the reaction stirred for 2 h. The reaction mixture was loaded onto an SCX-2 cartridge (20 g). The cartridge was washed with MeOH (2 CV), then the compound was eluted with 2 M ammonia in methanol (2 CV). The eluent was concentrated to dryness under reduced pressure to give crude product. This was suspended in DMSO and triturated with water before filtering and drying under vacuum to afford the desired product methyl 8-bromo-5,6-dihydrobenzo[f]imidazo[1,5-d][1,4]oxazepine-10-carboxylate (174 mg, 0.538 mmol, 44% yield) as a yellow solid. ¹H NMR (400 MHz, DMSO-D6) δ 8.30 (d, J=2.1 Hz, 1H), 7.98 (d, J=2.0 Hz, 1H), 7.84 (d, J=1.1 Hz, 1H), 7.60 (d, J=1.1 Hz, 1H), 4.63-4.56 (m, 2H), 4.56-4.49 (m, 2H), 3.86 (s, 3H). UPLC-MS (Basic 2 min): rt=0.99 min, m/z (ES+) 322.9, 324.8 [M+H]⁺.

### Lithium 8-bromo-5,6-dihydrobenzo[f]imidazo[1,5-d][1,4]oxazepine-10-carboxylate

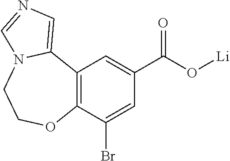

Lithium hydroxide (23 mg, 0.552 mmol) in water (1 mL) was added to a solution of methyl 8-bromo-5,6-dihydrobenzo[f]imidazo[1,5-d][1,4]oxazepine-10-carboxylate (170 mg, 0.526 mmol) in THF (4 mL) at 25° C. and stirred for 68 h. This was concentrated under reduced pressure to afford the desired product lithium 8-bromo-5,6-dihydrobenzo[f]imidazo[1,5-d][1,4]oxazepine-10-carboxylate (174 mg, 0.525 mmol, 99% yield) as an orange solid. ¹H NMR (400 MHz, DMSO-D6) δ 8.16 (d, J=1.8 Hz, 1H), 7.92 (d, J=1.8 Hz, 1H), 7.78 (d, J=1.1 Hz, 1H), 7.39 (d, J=1.1 Hz, 1H), 4.51-4.40 (m, 4H). UPLC-MS (Basic 2 min): rt=0.64 min, m/z (ES+) 308.9, 310.8 [M+H]⁺.

### Methyl 3-formyl-4-hydroxy-5-methylbenzoate

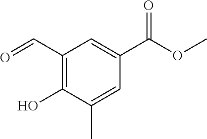

Hexamethylenetetramine (1.94 g, 13.8 mmol) was added to a suspension of methyl 4-hydroxy-3-methylbenzoate (500 mg, 3.01 mmol) and trifluoroacetic acid (6.9 mL, 90.0 mmol) and the mixture heated at 73° C. for 18 h. This was cooled to rt and water (35 mL) was slowly added before heating at 80° C. for 2 h. This was cooled and the resulting precipitate filtered and dried to yield methyl 3-formyl-4-hydroxy-5-methylbenzoate (317 mg, 1.29 mmol, 43% yield) as a beige solid. ¹H NMR (400 MHz, DMSO-D6) δ 11.46 (s, 1H), 10.14 (s, 1H), 8.26 (d, J=2.3 Hz, 1H), 8.03 (dd, J=2.3, 1.0 Hz, 1H), 3.85 (s, 3H), 2.25 (s, 3H). UPLC-MS (Basic 4 min): rt=0.87 min, m/z (ES+) 193.1 [M−H]⁻.

### Methyl (E)-4-hydroxy-3-(((2-hydroxyethyl)imino)methyl)-5-methylbenzoate

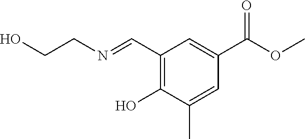 [SMILES: COC(=O)c1cc(C)c(O)c(/C=N/CCO)c1]

Ethanolamine (0.061 mL, 1.01 mmol) and methyl 3-formyl-4-hydroxy-5-methylbenzoate (249 mg, 1.01 mmol) in methanol (5 mL) were heated to 40° C. for 18 h. The reaction mixture was concentrated under reduced pressure to afford the desired product methyl (E)-4-hydroxy-3-(((2-hydroxyethyl)imino)methyl)-5-methylbenzoate (242 mg, 1.01 mmol, 100% yield) as a yellow solid. ¹H NMR (400 MHz, DMSO-D6) δ 8.60 (s, 1H), 7.92 (d, J=2.7 Hz, 1H), 7.75-7.72 (m, 1H), 3.78 (s, 3H), 3.69-3.62 (m, 4H), 2.13 (s, 3H). 2 OH not observed.

### Methyl 4-hydroxy-3-(1-(2-hydroxyethyl)-1H-imidazol-5-yl)-5-methylbenzoate

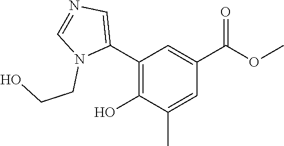 [SMILES: COC(=O)c1cc(C)c(O)c(-c2cncn2CCO)c1]

4-Toluenesulfonylmethylisocyanide (239 mg, 1.22 mmol) was added to a suspension of methyl (E)-4-hydroxy-3-(((2-hydroxyethyl)imino)methyl)-5-methylbenzoate (242 mg, 1.02 mmol) and potassium carbonate (310 mg, 2.24 mmol) in dimethoxyethane (2.5 mL) and methanol (1 mL) at 25° C. and stirred for 18 h. The reaction mixture was loaded onto an SCX-2 cartridge (20 g). The cartridge was washed with MeOH (2 CV), then the compound was eluted with 2 M ammonia in methanol (2 CV). The eluent was concentrated to dryness under reduced pressure to afford the desired product methyl 4-hydroxy-3-(1-(2-hydroxyethyl)-1H-imidazol-5-yl)-5-methylbenzoate (249 mg, 0.685 mmol, 67% yield) as a beige solid. ¹H NMR (400 MHz, DMSO-D6) δ 7.74 (dd, J=2.4, 0.8 Hz, 1H), 7.71 (d, J=1.1 Hz, 1H), 7.55 (d, J=2.3 Hz, 1H), 6.81 (d, J=1.1 Hz, 1H), 4.10 (s, 1H), 3.83 (t, J=5.7 Hz, 2H), 3.77 (s, 3H), 3.46 (t, J=5.8 Hz, 2H), 2.22 (s, 3H). 1 OH not observed. UPLC-MS (Basic 4 min): rt=0.93 min, m/z (ES+) 277.0 [M+H]⁺.

### Methyl 8-methyl-5,6-dihydrobenzo[f]imidazo[1,5-d][1,4]oxazepine-10-carboxylate

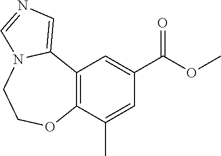 [SMILES: COC(=O)c1cc(C)c2c(c1)-c1cncn1CCO2]

Diisopropyl azodicarboxylate (0.27 mL, 1.36 mmol) was added to a solution of methyl 4-hydroxy-3-(1-(2-hydroxyethyl)-1H-imidazol-5-yl)-5-methylbenzoate (249 mg, 0.90 mmol) and triphenylphosphine (284 mg, 1.08 mmol) in THF (4.5 mL) and the reaction stirred at rt for 18 h. The reaction mixture was loaded onto an SCX-2 cartridge (10 g). The cartridge was washed with MeOH (2 CV), then the compound was eluted with 2 M ammonia in methanol (2 CV). The eluent was concentrated to dryness under reduced pressure to give crude product. This was purified by column chromatography over silica eluting with a gradient of MeOH (0% to 10%; v/v) in DCM to afford crude product. This was purified by column chromatography over C18 eluting with a gradient of MeCN(0.1% NH₃) (5% to 90%; v/v) in water (0.1% NH₃) to afford the desired product methyl 8-methyl-5,6-dihydrobenzo[f]imidazo[1,5-d][1,4]oxazepine-10-carboxylate (97 mg, 0.376 mmol, 42% yield) as an off-white solid. ¹H NMR (400 MHz, DMSO-D6) δ 8.17 (dd, J=2.2, 0.6 Hz, 1H), 7.79 (d, J=1.1 Hz, 1H), 7.68 (dd, J=2.2, 0.9 Hz, 1H), 7.49 (d, J=1.1 Hz, 1H), 4.52-4.46 (m, 4H), 3.84 (s, 3H), 2.25 (s, 3H). UPLC-MS (Basic 4 min): rt=1.43 min, m/z (ES+) 259.1 [M+H]⁺.

### Lithium 8-methyl-5,6-dihydrobenzo[f]imidazo[1,5-d][1,4]oxazepine-10-carboxylate

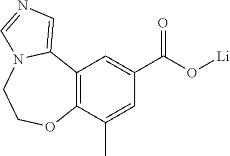

Lithium hydroxide (18 mg, 0.432 mmol) in water (1 mL) was added to a solution of methyl 8-methyl-5,6-dihydrobenzo[f]imidazo[1,5-d][1,4]oxazepine-10-carboxylate (97 mg, 0.376 mmol) in THF (4 mL) at 25° C. and stirred for 18 h. Further lithium hydroxide (18 mg, 0.432 mmol) was added and the reaction stirred for 5 h. This was concentrated under reduced pressure to afford the desired product lithium 8-methyl-5,6-dihydrobenzo[f]imidazo[1,5-d][1,4]oxazepine-10-carboxylate (120 mg, 0.376 mmol, 99% yield) as a beige solid. ¹H NMR (400 MHz, DMSO-D6) δ 8.01 (d, J=2.0 Hz, 1H), 7.72 (d, J=1.1 Hz, 1H), 7.59-7.55 (m, 1H), 7.30 (d, J=1.1 Hz, 1H), 4.39 (s, 4H), 2.19 (s, 3H). UPLC-MS (Basic 4 min): rt=0.68 min, m/z (ES+) 243.1 [M−H]⁻.

### Methyl 3-formyl-4-hydroxy-5-methoxybenzoate

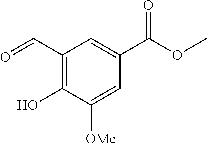

Hexamethylenetetramine (1.77 g, 12.6 mmol) was added to a suspension of methyl 4-hydroxy-3-methoxy benzoate (500 mg, 2.74 mmol) and trifluoroacetic acid (6.3 mL, 82.3 mmol) and the mixture heated at 73° C. for 18 h. This was cooled to rt and water (31 mL) was slowly added before heating at 80° C. for 2 h. This was cooled and the resulting precipitate filtered and dried to yield methyl 3-formyl-4-hydroxy-5-methoxybenzoate (388 mg, 1.85 mmol, 67% yield) as a white solid. ¹H NMR (400 MHz, DMSO-D6) δ 11.08 (s, 1H), 10.32 (s, 1H), 7.90 (d, J=2.0 Hz, 1H), 7.65 (d, J=2.0 Hz, 1H), 3.93 (s, 3H), 3.84 (s, 3H). UPLC-MS (Basic 4 min): rt=0.68 min, m/z (ES+) 210.1 [M+H]+

### Methyl (E)-4-hydroxy-3-(((2-hydroxyethyl)imino)methyl)-5-methoxybenzoate

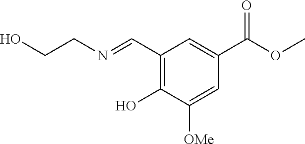 [SMILES: COC(=O)c1cc(/C=N/CCO)c(O)c(OC)c1]

Ethanolamine (0.11 mL, 1.85 mmol) and methyl 3-formyl-4-hydroxy-5-methoxybenzoate (388 mg, 1.85 mmol) in methanol (9 mL) were heated to 40° C. for 96 h. The reaction mixture was concentrated under reduced pressure to afford the desired product methyl (E)-4-hydroxy-3-(((2-hydroxyethyl)imino)methyl)-5-methoxybenzoate (466 mg, 1.84 mmol, 100% yield) as a yellow solid. ¹H NMR (400 MHz, DMSO-D6) δ 8.55 (s, 1H), 7.69 (d, J=2.0 Hz, 1H), 7.19 (d, J=2.1 Hz, 1H), 5.05-4.98 (m, 1H), 3.77 (s, 3H), 3.74 (s, 3H), 3.67-3.62 (m, 4H). 1 OH not observed.

### Methyl 4-hydroxy-3-(1-(2-hydroxyethyl)-1H-imidazol-5-yl)-5-methoxybenzoate

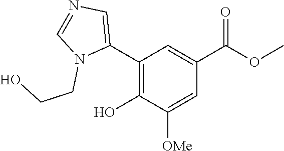 [SMILES: COC(=O)c1cc(OC)c(O)c(-c2cncn2CCO)c1]

4-Toluenesulfonylmethylisocyanide (431 mg, 2.21 mmol) was added to a suspension of methyl (E)-4-hydroxy-3-(((2-hydroxyethyl)imino)methyl)-5-methoxybenzoate (466 mg, 1.84 mmol) and potassium carbonate (560 mg, 4.05 mmol) in dimethoxyethane (7.5 mL) and methanol (3 mL) at 25° C. and stirred for 18 h. The reaction mixture was loaded onto an SCX-2 cartridge (20 g). The cartridge was washed with MeOH (2 CV), then the compound was eluted with 2 M ammonia in methanol (2 CV). The eluent was concentrated to dryness under reduced pressure to afford the desired product methyl 4-hydroxy-3-(1-(2-hydroxyethyl)-1H-imidazol-5-yl)-5-methoxybenzoate (534 mg, 1.75 mmol, 95% yield) as a beige solid. ¹H NMR (400 MHz, DMSO-D6) δ 7.71 (d, J=1.1 Hz, 1H), 7.52 (d, J=2.0 Hz, 1H), 7.42 (d, J=2.0 Hz, 1H), 6.84 (d, J=1.1 Hz, 1H), 3.91 (s, 3H), 3.86 (t, J=5.8 Hz, 2H), 3.81 (s, 3H), 3.41 (t, J=5.8 Hz, 2H). 2 OH not observed. UPLC-MS (Basic 4 min): rt=0.74 min, m/z (ES+) 293.3 [M+H]⁺.

### Methyl 8-methoxy-5,6-dihydrobenzo[f]imidazo[1,5-d][1,4]oxazepine-10-carboxylate

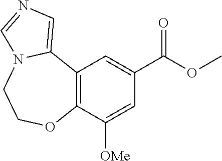 [SMILES: COC(=O)c1cc(OC)c2c(c1)-c1cncn1CCO2]

Diisopropyl azodicarboxylate (0.54 mL, 2.74 mmol) was added to a solution of methyl 4-hydroxy-3-(1-(2-hydroxyethyl)-1H-imidazol-5-yl)-5-methoxybenzoate (534 mg, 1.83 mmol) and triphenylphosphine (575 mg, 2.19 mmol) in THF (9 mL) and the reaction stirred at rt for 18 h. The reaction mixture was loaded onto an SCX-2 cartridge (10 g). The cartridge was washed with MeOH (2 CV), then the compound was eluted with 2 M ammonia in methanol (2 CV). The eluent was concentrated to dryness under reduced pressure to give crude product. This was purified by column chromatography over C18 eluting with a gradient of MeCN(0.1% NH₃) (5% to 90%; v/v) in water (0.1% NH₃) to afford the desired product methyl 8-methoxy-5,6-dihydrobenzo[f]imidazo[1,5-d][1,4]oxazepine-10-carboxylate (274 mg, 0.959 mmol, 52% yield) as an off-white solid. ¹H NMR (400 MHz, DMSO-D6) δ 7.94 (d, J=2.0 Hz, 1H), 7.80 (d, J=1.1 Hz, 1H), 7.51 (d, J=1.1 Hz, 1H), 7.35 (d, J=2.0 Hz, 1H), 4.48 (s, 4H), 3.86 (s, 3H), 3.84 (s, 3H). UPLC-MS (Basic 4 min): rt=1.18 min, m/z (ES+) 275.0 [M+H]⁺.

### Lithium 8-methoxy-5,6-dihydrobenzo[f]imidazo[1,5-d][1,4]oxazepine-10-carboxylate

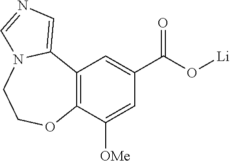

Lithium hydroxide (48 mg, 1.15 mmol) in water (1 mL) was added to a solution of methyl 8-methoxy-5,6-dihydrobenzo[f]imidazo[1,5-d][1,4]oxazepine-10-carboxylate (274 mg, 0.999 mmol) in THF (4 mL) at 25° C. and stirred for 68 h. This was concentrated under reduced pressure to afford the desired product lithium 8-methoxy-5,6-dihydrobenzo[f]imidazo[1,5-d][1,4]oxazepine-10-carboxylate (287 mg, 0.999 mmol, 99% yield) as an orange solid. ¹H NMR (400 MHz, DMSO-D6) δ 8.01 (d, J=2.0 Hz, 1H), 7.72 (d, J=1.1 Hz, 1H), 7.59-7.55 (m, 1H), 7.30 (d, J=1.1 Hz, 1H), 4.39 (s, 4H), 2.19 (s, 3H). UPLC-MS (Basic 4 min): rt=0.68 min, m/z (ES+) 243.1 [M+H]⁻.

### Methyl 3-formyl-4-hydroxy-5-(trifluoromethyl)benzoate

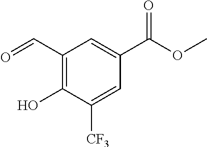 [SMILES: COC(=O)c1cc(C=O)c(O)c(C(F)(F)F)c1]

Hexamethylenetetramine (1.46 g, 10.4 mmol) was added to a suspension of methyl 4-hydroxy-3-(trifluoromethyl)benzoate (500 mg, 2.27 mmol) and trifluoroacetic acid (5.3 mL, 68.6 mmol) and the mixture heated at 73° C. for 42 h. This was cooled to rt and water (30 mL) was slowly added before stirring at 25° C. for 1 h. This was cooled and the resulting precipitate filtered to give crude product. This was purified by column chromatography over silica eluting with a gradient of EtOAc (0% to 50%; v/v) in iso-hexane to afford the desired product methyl 3-formyl-4-hydroxy-5-(trifluoromethyl)benzoate (278 mg, 1.09 mmol, 48% yield) as a white solid. ¹H NMR (400 MHz, DMSO-D6) δ 10.19 (s, 1H), 8.60 (d, J=2.3 Hz, 1H), 8.29 (d, J=2.2 Hz, 1H), 3.88 (s, 3H). OH not observed. UPLC-MS (Basic 2 min): rt=0.76 min, m/z (ES+) 247.1 [M−H]⁻.

### Methyl (E)-4-hydroxy-3-(((2-hydroxyethyl)imino)methyl)-5-(trifluoromethyl)benzoate

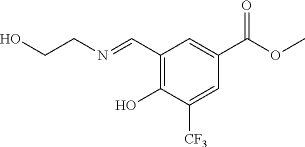 [SMILES: COC(=O)c1cc(/C=N/CCO)c(O)c(C(F)(F)F)c1]

Ethanolamine (0.066 mL, 1.09 mmol) and methyl 3-formyl-4-hydroxy-5-(trifluoromethyl)benzoate (278 mg, 1.09 mmol) in methanol (5 mL) were heated to 40° C. for 1 h. The reaction mixture was concentrated under reduced pressure to afford the desired product methyl (E)-4-hydroxy-3-(((2-hydroxyethyl)imino)methyl)-5-(trifluoromethyl)benzoate (320 mg, 1.07 mmol, 98% yield) as a yellow solid. ¹H NMR (400 MHz, DMSO-D6) δ 8.69 (s, 1H), 8.24 (d, J=2.5 Hz, 1H), 7.98 (d, J=2.5 Hz, 1H), 5.02 (s, 1H), 3.77 (s, 3H), 3.65 (s, 4H). 1 OH not observed.

### Methyl 4-hydroxy-3-(1-(2-hydroxyethyl)-1H-imidazol-5-yl)-5-(trifluoromethyl)benzoate

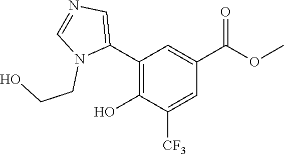 [SMILES: COC(=O)c1cc(-c2cncn2CCO)c(O)c(C(F)(F)F)c1]

4-Toluenesulfonylmethylisocyanide (250 mg, 1.28 mmol) was added to a suspension of methyl (E)-4-hydroxy-3-(((2-hydroxyethyl)imino)methyl)-5-(trifluoromethyl)benzoate (320 mg, 1.07 mmol) and potassium carbonate (324 mg, 2.34 mmol) in dimethoxyethane (5 mL) and methanol (3 mL) at 25° C. and stirred for 18 h. The reaction mixture was loaded onto an SCX-2 cartridge (10 g). The cartridge was washed with MeOH (2 CV), then the compound was eluted with 2 M ammonia in methanol (2 CV). The eluent was concentrated to dryness under reduced pressure to give crude product. This was purified by column chromatography over C18 eluting with a gradient of MeCN(0.1% NH₃) (5% to 40%; v/v) in water (0.1% NH₃) to afford the desired product methyl 4-hydroxy-3-(1-(2-hydroxyethyl)-1H-imidazol-5-yl)-5-(trifluoromethyl)benzoate (255 mg, 0.757 mmol, 71% yield) as a white solid. ¹H NMR (400 MHz, DMSO-D6) δ 8.58 (s, 1H), 7.94 (d, J=2.5 Hz, 1H), 7.74 (d, J=2.6 Hz, 1H), 7.32 (d, J=1.4 Hz, 1H), 4.03 (t, J=5.3 Hz, 2H), 3.73 (s, 3H), 3.55 (t, J=5.4 Hz, 2H). 2 OH not observed. UPLC-MS (Basic 2 min): rt=0.74 min, m/z (ES+) 330.9 [M+H]⁺.

### Methyl 8-(trifluoromethyl)-5,6-dihydrobenzo[f]imidazo[1,5-d][1,4]oxazepine-10-carboxylate

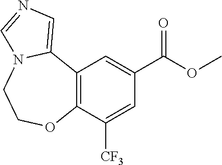 [SMILES: COC(=O)c1cc2c(c(C(F)(F)F)c1)OCCn1cncc1-2]

Diisopropyl azodicarboxylate (0.22 mL, 1.12 mmol) was added to a solution of methyl 4-hydroxy-3-(1-(2-hydroxyethyl)-1H-imidazol-5-yl)-5-(trifluoromethyl)benzoate (252 mg, 0.748 mmol) and triphenylphosphine (235 mg, 0.897 mmol) in THF (13 mL) and the reaction stirred at rt for 18 h. The reaction mixture was loaded onto an SCX-2 cartridge (10 g). The cartridge was washed with MeOH (2 CV), then the compound was eluted with 2 M ammonia in methanol (2 CV). The eluent was concentrated to dryness under reduced pressure to afford the desired product methyl 8-(trifluoromethyl)-5,6-dihydrobenzo[f]imidazo[1,5-d][1,4]oxazepine-10-carboxylate (195 mg, 0.624 mmol, 84% yield) as an off-white solid. ¹H NMR (400 MHz, DMSO-D6) δ 7.74 (d, J=2.1 Hz, 1H), 7.17 (d, J=2.1 Hz, 1H), 7.05 (d, J=1.0 Hz, 1H), 6.86 (d, J=1.1 Hz, 1H), 3.83-3.69 (m, 4H), 3.07 (s, 3H). UPLC-MS (Basic 2 min): rt=1.01 min, m/z (ES+) 313.2 [M+H]⁺.

### Lithium 8-(trifluoromethyl)-5,6-dihydrobenzo[f]imidazo[1,5-d][1,4]oxazepine-10-carboxylate

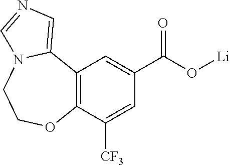

Lithium hydroxide (27 mg, 0.639 mmol) in water (1 mL) was added to a solution of methyl 8-(trifluoromethyl)-5,6-dihydrobenzo[f]imidazo[1,5-d][1,4]oxazepine-10-carboxylate (170 mg, 0.526 mmol) in THF (4 mL) at 25° C. and stirred for 18 h. This was concentrated under reduced pressure to afford the desired product lithium 8-(trifluoromethyl)-5,6-dihydrobenzo[f]imidazo[1,5-d][1,4]oxazepine-10-carboxylate (174 mg, 0.525 mmol, 99% yield) as a purple solid. ¹H NMR (400 MHz, DMSO-D6) δ 8.44 (d, J=1.9 Hz, 1H), 8.00 (d, J=1.9 Hz, 1H), 7.82 (d, J=1.1 Hz, 1H), 7.46 (d, J=1.1 Hz, 1H), 4.49 (q, J=2.0 Hz, 4H). UPLC-MS (Basic 2 min): rt=0.67 min, m/z (ES+) 299.2 [M+H]⁺.

### Methyl 3-fluoro-5-formyl-4-hydroxybenzoate

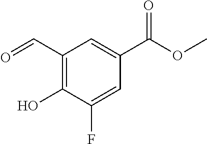

Prepared from methyl 3-fluoro-4-hydroxybenzoate according to General Procedure F to afford the desired product methyl 3-fluoro-5-formyl-4-hydroxy benzoate (385 mg, 1.94 mmol, 66% yield) as a yellow solid. ¹H NMR (400 MHz, DMSO-D6) δ 11.93 (s, 1H), 10.31 (s, 1H), 8.09 (dd, J=2.2, 1.1 Hz, 1H), 7.95 (dd, J=11.2, 2.1 Hz, 1H), 3.85 (s, 3H). UPLC-MS (Basic 2 min): rt=0.63 min, m/z (ES+) 197.1 [M−H]

### Methyl (E)-3-fluoro-4-hydroxy-5-(((2-hydroxyethyl)imino)methyl)benzoate

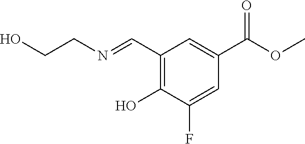 [SMILES: COC(=O)c1cc(F)c(O)c(/C=N/CCO)c1]

Prepared from ethanolamine and methyl 3-fluoro-5-formyl-4-hydroxy benzoate according to General Procedure A to afford the desired product methyl (E)-3-fluoro-4-hydroxy-5-(((2-hydroxyethyl)imino)methyl)benzoate (460 mg, 1.91 mmol, 98% yield) as a yellow solid. ¹H NMR (400 MHz, DMSO-D6) δ 13.62 (s, 1H), 8.66 (d, J=1.3 Hz, 1H), 7.88 (dd, J=2.4, 0.8 Hz, 1H), 7.50 (dd, J=12.7, 2.3 Hz, 1H), 5.11 (s, 1H), 3.77 (s, 3H), 3.75-3.63 (m, 4H).

### Methyl 3-fluoro-4-hydroxy-5-(1-(2-hydroxyethyl)-1H-imidazol-5-yl)benzoate

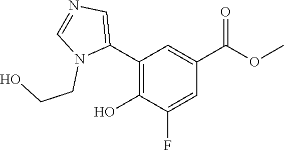 [SMILES: COC(=O)c1cc(F)c(O)c(-c2cncn2CCO)c1]

Prepared from methyl (E)-3-fluoro-4-hydroxy-5-(((2-hydroxyethyl)imino)methyl)benzoate according to General Procedure B to afford the desired product methyl 3-fluoro-4-hydroxy-5-(1-(2-hydroxyethyl)-1H-imidazol-5-yl)benzoate (530 mg, 1.80 mmol, 94% yield) as an off-white solid. ¹H NMR (400 MHz, DMSO-D6) δ 7.72 (d, J=1.2 Hz, 1H), 7.60 (dd, J=11.7, 2.2 Hz, 1H), 7.52 (dd, J=2.2, 0.9 Hz, 1H), 6.82 (d, J=1.2 Hz, 1H), 3.90 (t, J=5.7 Hz, 2H), 3.77 (s, 3H), 3.48 (t, J=5.7 Hz, 2H), 3.17 (s, 1H). 1 OH not observed. UPLC-MS (Basic 2 min): rt=0.63 min, m/z (ES+) 281.0 [M+H]⁺.

### Methyl 8-fluoro-5,6-dihydrobenzo[f]imidazo[1,5-d][1,4]oxazepine-10-carboxylate

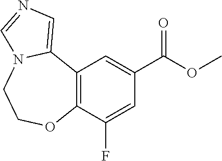 [SMILES: COC(=O)c1cc(F)c2c(c1)-c1cncn1CCO2]

Prepared from methyl 3-fluoro-4-hydroxy-5-(1-(2-hydroxyethyl)-1H-imidazol-5-yl)benzoate according to General Procedure C to afford the desired product methyl 8-fluoro-5,6-dihydrobenzo[f]imidazo[1,5-d][1,4]oxazepine-10-carboxylate (408 mg, 1.48 mmol, 82% yield) as an off-white solid. ¹H NMR (400 MHz, DMSO-D6) δ 8.15 (t, J=1.8 Hz, 1H), 7.83 (d, J=1.1 Hz, 1H), 7.63-7.57 (m, 2H), 4.61-4.51 (m, 4H), 3.86 (s, 3H). UPLC-MS (Basic 2 min): rt=0.91 min, m/z (ES+) 263.1 [M+H]⁺.

### Lithium 8-fluoro-5,6-dihydrobenzo[f]imidazo[1,5-d][1,4]oxazepine-10-carboxylate

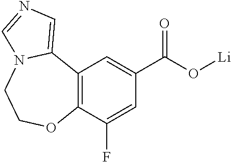

Prepared from methyl 8-fluoro-5,6-dihydrobenzo[f]imidazo[1,5-d][1,4]oxazepine-10-carboxylate according to General Procedure D to afford the desired product lithium 8-fluoro-5,6-dihydrobenzo[f]imidazo[1,5-d][1,4]oxazepine-10-carboxylate (390 mg, 1.46 mmol, 99% yield) as a green solid. ¹H NMR (400 MHz, DMSO-D6) δ 8.05 (t, J=1.7 Hz, 1H), 7.77 (d, J=1.1 Hz, 1H), 7.47 (dd, J=11.5, 1.8 Hz, 1H), 7.43 (d, J=1.1 Hz, 1H), 4.52-4.43 (m, 4H).

### Methyl (1r,4r)-4-(5,6-dihydrobenzo[f]imidazo[1,5-d][1,4]oxazepine-10-carboxamido)cyclohexane-1-carboxylate 30

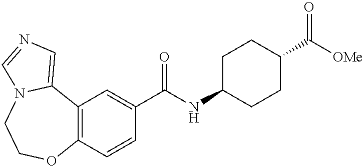 [SMILES: COC(=O)[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and trans-methyl 4-aminocyclohexanecarboxylate hydrochloride according to General Procedure E to afford the desired product methyl (1r,4r)-4-(5,6-dihydrobenzo[f]imidazo[1,5-d][1,4]oxazepine-10-carboxamido)cyclohexane-1-carboxylate 30 (203 mg, 0.544 mmol, 71% yield) as a white solid. ¹H NMR (400 MHz, DMSO-D6) δ 8.26-8.20 (m, 2H), 7.78 (d, J=1.1 Hz, 1H), 7.66 (dd, J=8.6, 2.2 Hz, 1H), 7.61 (d, J=1.1 Hz, 1H), 7.04 (d, J=8.5 Hz, 1H), 4.51-4.39 (m, 4H), 3.77-3.72 (m, 1H), 3.60 (s, 3H), 2.00-1.87 (m, 5H), 1.51-1.31 (m, 4H). 1H not observed. UPLC-MS (Basic 2 min): rt=0.89 min, m/z (ES+) 370.2 [M+H]⁺.

### Methyl (1r,4r)-4-(5,6-dihydrobenzo[f]imidazo[1,5-d][1,4]oxazepine-10-carboxamido)cyclohexane-1-carboxylate 31

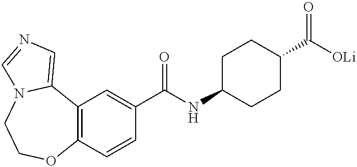

Prepared from methyl (1r,4r)-4-(5,6-dihydrobenzo[f]imidazo[1,5-d][1,4]oxazepine-10-carboxamido)cyclohexane-1-carboxylate 30 according to General Procedure D to afford the desired product methyl (1r,4r)-4-(5,6-dihydrobenzo[f]imidazo[1,5-d][1,4]oxazepine-10-carboxamido)cyclohexane-1-carboxylate 31 (195 mg, 0.513 mmol, 96% yield) as a yellow solid. ¹H NMR (400 MHz, DMSO-D6) δ 8.25 (d, J=2.2 Hz, 1H), 8.16 (s, 1H), 7.76 (s, 1H), 7.66 (dd, J=8.6, 2.2 Hz, 1H), 7.62 (d, J=1.1 Hz, 1H), 7.03 (d, J=8.6 Hz, 1H), 4.51-4.39 (m, 4H), 3.75-3.65 (m, 1H), 1.90-1.78 (m, 4H), 1.78-1.69 (m, 1H), 1.38-1.22 (m, 4H). UPLC-MS (Basic 2 min): rt=0.63 min, m/z (ES+) 356.3 [M+H]+

### Methyl (E)-4-hydroxy-3-(((1-hydroxypropan-2-yl)imino)methyl)benzoate

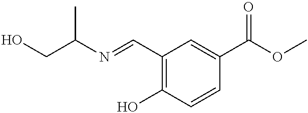 [SMILES: COC(=O)c1ccc(O)c(/C=N/C(C)CO)c1]

Prepared from 2-amino-1-propanol and methyl 3-formyl-4-hydroxy benzoate according to General Procedure A to afford the desired product methyl (E)-4-hydroxy-3-(((1-hydroxypropan-2-yl)imino)methyl)benzoate (440 mg, 1.72 mmol, 99% yield) as a red solid. 1H NMR (400 MHz, DMSO-D6) δ 8.66 (s, 1H), 8.10 (d, J=2.3 Hz, 1H), 7.83 (dd, J=8.9, 2.4 Hz, 1H), 6.79 (d, J=8.9 Hz, 1H), 4.97 (s, 1H), 3.80 (s, 3H), 3.63-3.52 (m, 2H), 3.46-3.39 (m, 1H), 1.23 (d, J=6.4 Hz, 3H).

### Methyl 4-hydroxy-3-(1-(1-hydroxypropan-2-yl)-1H-imidazol-5-yl)benzoate

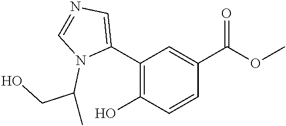 [SMILES: COC(=O)c1ccc(O)c(-c2cncn2C(C)CO)c1]

Prepared from methyl (E)-4-hydroxy-3-(((1-hydroxypropan-2-yl)imino)methyl)benzoate according to General Procedure B to afford the desired product methyl 4-hydroxy-3-(1-(1-hydroxypropan-2-yl)-1H-imidazol-5-yl)benzoate (456 mg, 0.844 mmol, 77% yield) as a red solid. ¹H NMR (400 MHz, DMSO-D6) δ 7.84 (dd, J=8.6, 2.3 Hz, 1H), 7.81 (d, J=1.1 Hz, 1H), 7.72 (d, J=2.3 Hz, 1H), 6.97 (d, J=8.5 Hz, 1H), 6.79 (d, J=1.1 Hz, 1H), 3.94-3.88 (m, 1H), 3.79 (s, 3H), 3.46 (dd, J=5.9, 2.6 Hz, 2H), 1.36 (d, J=6.9 Hz, 3H). 2 OH not observed. UPLC-MS (Basic 4 min): rt=0.82 min, m/z (ES+) 277.1 [M+H]⁺.

### Methyl 5-methyl-5,6-dihydrobenzo[f]imidazo[1,5-d][1,4]oxazepine-10-carboxylate

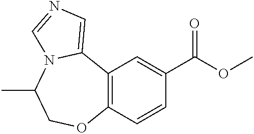

Prepared from methyl 4-hydroxy-3-(1-(1-hydroxypropan-2-yl)-1H-imidazol-5-yl)benzoate according to General Procedure C to afford the desired product methyl 5-methyl-5,6-dihydrobenzo[f]imidazo[1,5-d][1,4]oxazepine-10-carboxylate (182 mg, 0.691 mmol, 42% yield) as an off-white solid. ¹H NMR (400 MHz, DMSO-D6) δ 8.36 (d, J=2.1 Hz, 1H), 7.87 (d, J=1.1 Hz, 1H), 7.73 (dd, J=8.6, 2.1 Hz, 1H), 7.54 (d, J=1.1 Hz, 1H), 7.13 (d, J=8.6 Hz, 1H), 4.89-4.81 (m, 1H), 4.51 (dd, J=12.9, 4.6 Hz, 1H), 4.32 (dd, J=12.9, 1.4 Hz, 1H), 3.85 (s, 3H), 1.44 (d, J=6.9 Hz, 3H). UPLC-MS (Basic 4 min): rt=1.39 min, m/z (ES+) 259.1 [M+H]⁺.

### Lithium 5,5-dimethyl-5,6-dihydrobenzo[f]imidazo[1,5-d][1,4]oxazepine-10-carboxylate

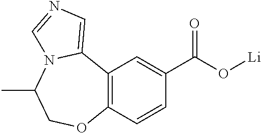

Prepared from methyl 5-methyl-5,6-dihydrobenzo[f]imidazo[1,5-d][1,4]oxazepine-10-carboxylate according to General Procedure D to afford the desired product lithium 5,5-dimethyl-5,6-dihydrobenzo[f]imidazo[1,5-d][1,4]oxazepine-10-carboxylate (190 mg, 0.729 mmol, 100% yield) as a beige solid. ¹H NMR (400 MHz, DMSO-D6) δ 8.26 (d, J=1.9 Hz, 1H), 7.80 (d, J=1.1 Hz, 1H), 7.63 (dd, J=8.3, 1.9 Hz, 1H), 7.37 (d, J=1.2 Hz, 1H), 6.87 (d, J=8.3 Hz, 1H), 4.82-4.73 (m, 1H), 4.40 (dd, J=12.8, 4.7 Hz, 1H), 4.21 (dd, J=12.8, 1.5 Hz, 1H), 1.44 (d, J=6.8 Hz, 3H). UPLC-MS (Basic 2 min): rt=0.64 min, m/z (ES+) 243.1 [M+H]⁺.

### 5-(2-(1H-Imidazol-5-yl)ethoxy)-6-bromo-N-((1r,4r)-4-(2-methoxyethoxy)cyclohexyl) picolinamide

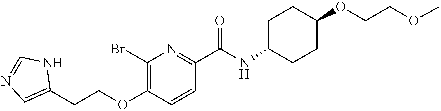 [SMILES: COCCO[C@H]1CC[C@H](NC(=O)c2ccc(OCCc3cnc[nH]3)c(Br)n2)CC1]

Prepared from lithium 5-(2-(1H-imidazol-5-yl)ethoxy)-6-bromopicolinate 28 and trans-4-(2-methoxyethoxy)cyclohexanamine according to General Procedure E, using DMSO as a co-solvent, to afford the desired product 5-(2-(1H-imidazol-5-yl)ethoxy)-6-bromo-N-((1r,4r)-4-(2-methoxyethoxy)cyclohexyl) picolinamide (23 mg, 0.0457 mmol, 21% yield) as a brown solid. UPLC-MS (Basic 4 min): rt=1.35 min, m/z (ES+) 467.4, 469.4 [M+H]⁺.

### Methyl 2,5-difluoro-3-formyl-4-hydroxybenzoate

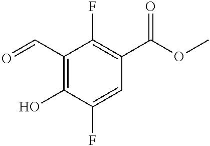

Prepared from methyl 2,5-difluoro-4-hydroxybenzoate according to General Procedure F to afford the desired product methyl 2,5-difluoro-3-formyl-4-hydroxy benzoate (76 mg, 0.341 mmol, 13% yield) as a beige solid. ¹H NMR (400 MHz, DMSO-D6) δ 10.24 (d, J=1.0 Hz, 1H), 7.95-7.86 (m, 1H), 3.83 (s, 3H). 1H not observed. UPLC-MS (Basic 2 min): rt=0.62 min, m z (ES+) 215.0 [M−H]⁻.

### Methyl (E)-2,5-difluoro-4-hydroxy-3-(((2-hydroxyethyl)imino)methyl)benzoate

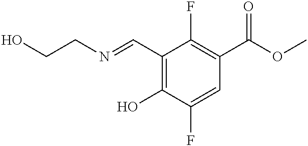 [SMILES: COC(=O)c1cc(F)c(O)c(/C=N/CCO)c1F]

Prepared from ethanolamine and methyl 2,5-difluoro-3-formyl-4-hydroxy benzoate according to General Procedure A to afford the desired product methyl (E)-2,5-difluoro-4-hydroxy-3-(((2-hydroxyethyl)imino)methyl)benzoate (90 mg, 0.335 mmol, 95% yield) as a yellow solid. ¹H NMR (400 MHz, DMSO-D6) δ 8.74 (d, J=1.3 Hz, 1H), 7.44 (dd, J=12.6, 7.9 Hz, 1H), 5.12 (t, J=5.2 Hz, 1H), 3.79-3.74 (m, 5H), 3.69-3.59 (m, 2H). 1H not observed.

### Methyl 2,5-difluoro-4-hydroxy-3-(1-(2-hydroxyethyl)-1H-imidazol-5-yl)benzoate

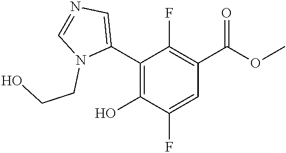 [SMILES: COC(=O)c1cc(F)c(O)c(-c2cncn2CCO)c1F]

Prepared from methyl (E)-2,5-difluoro-4-hydroxy-3-(((2-hydroxyethyl)imino)methyl)benzoate according to General Procedure B to afford the desired product methyl 2,5-difluoro-4-hydroxy-3-(1-(2-hydroxyethyl)-1H-imidazol-5-yl)benzoate (96 mg, 0.312 mmol, 90% yield) as an off-white solid. ¹H NMR (400 MHz, DMSO-D6) δ 7.79 (s, 1H), 7.39-7.30 (m, 1H), 7.11 (s, 1H), 6.76 (s, 1H), 3.81 (t, J=5.7 Hz, 2H), 3.70 (s, 3H), 3.51 (t, J=5.7 Hz, 2H), 3.17 (d, J=5.0 Hz, 1H). UPLC-MS (Basic 2 min): rt=0.62 min, m/z (ES+) 299.1 [M+H]⁺.

### Methyl 8,11-difluoro-5,6-dihydrobenzo[f]imidazo[1,5-d][1,4]oxazepine-10-carboxylate

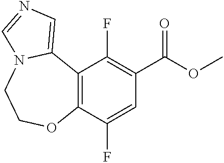 [SMILES: COC(=O)c1cc(F)c2c(c1F)-c1cncn1CCO2]

Prepared from methyl 2,5-difluoro-4-hydroxy-3-(1-(2-hydroxyethyl)-1H-imidazol-5-yl)benzoate according to General Procedure C to afford the desired product methyl 8,11-difluoro-5,6-dihydrobenzo[f]imidazo[1,5-d][1,4]oxazepine-10-carboxylate (90 mg, 0.305 mmol, 99% yield) as an off-white solid. ¹H NMR (400 MHz, DMSO-D6) δ 7.92 (d, J=1.0 Hz, 1H), 7.66 (dd, J=10.6, 6.7 Hz, 1H), 7.44 (dd, J=5.8, 1.0 Hz, 1H), 4.66-4.60 (m, 2H), 4.56-4.49 (m, 2H), 3.86 (s, 3H). UPLC-MS (Basic 2 min): rt=0.90 min, m/z (ES+) 281.2 [M+H]⁺.

### Lithium 8,11-difluoro-5,6-dihydrobenzo[f]imidazo[1,5-d][1,4]oxazepine-10-carboxylate

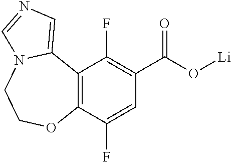

Prepared from methyl 8,11-difluoro-5,6-dihydrobenzo[f]imidazo[1,5-d][1,4]oxazepine-10-carboxylate according to General Procedure D to afford the desired product lithium 8,11-difluoro-5,6-dihydrobenzo[f]imidazo[1,5-d][1,4]oxazepine-10-carboxylate (100 mg, 0.294 mmol, 96% yield) as a red solid. ¹H NMR (400 MHz, DMSO-D6) δ 7.86 (d, J=1.0 Hz, 1H), 7.29 (dd, J=4.5, 1.0 Hz, 1H), 7.25 (dd, J=10.7, 6.5 Hz, 1H), 4.53-4.47 (m, 2H), 4.42-4.35 (m, 2H).

### Methyl 3-chloro-5-formyl-4-hydroxybenzoate

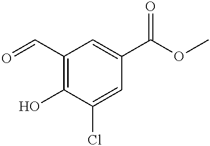

Prepared from methyl 3-chloro-4-hydroxy benzoate according to General Procedure F to afford the desired product methyl 3-chloro-5-formyl-4-hydroxybenzoate (423 mg, 1.73 mmol, 65% yield) as a yellow solid. ¹H NMR (400 MHz, DMSO-D6) δ 10.22 (s, 1H), 8.29 (d, J=2.2 Hz, 1H), 8.17 (d, J=2.2 Hz, 1H), 3.86 (s, 3H). UPLC-MS (Basic 2 min): rt=0.66 min, m z (ES+) 213.1, 215.0 [M−H]⁻.

### Methyl (E)-3-chloro-4-hydroxy-5-(((2-hydroxyethyl)imino)methyl)benzoate

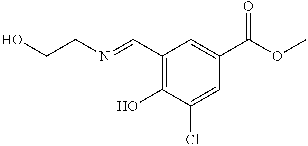 [SMILES: COC(=O)c1cc(Cl)c(O)c(/C=N/CCO)c1]

Prepared from ethanolamine and methyl 3-chloro-5-formyl-4-hydroxybenzoate according to General Procedure A to afford the desired product methyl (E)-3-chloro-4-hydroxy-5-(((2-hydroxyethyl)imino)methyl)benzoate (500 mg, 1.71 mmol, 98% yield) as a yellow solid. ¹H NMR (400 MHz, DMSO-D6) δ 13.87 (s, 1H), 8.69 (s, 1H), 8.00 (d, J=2.4 Hz, 1H), 7.88 (d, J=2.3 Hz, 1H), 5.13 (s, 1H), 3.77 (s, 3H), 3.73-3.62 (m, 4H).

### Methyl 3-chloro-4-hydroxy-5-(1-(2-hydroxyethyl)-1H-imidazol-5-yl)benzoate

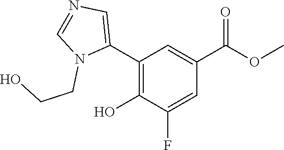 [SMILES: COC(=O)c1cc(F)c(O)c(-c2cncn2CCO)c1]

Prepared from methyl (E)-3-chloro-4-hydroxy-5-(((2-hydroxyethyl)imino)methyl)benzoate according to General Procedure B to afford the desired product methyl 3-chloro-4-hydroxy-5-(1-(2-hydroxyethyl)-1H-imidazol-5-yl)benzoate (400 mg, 1.29 mmol, 76% yield) as a white solid. ¹H NMR (400 MHz, DMSO-D6) δ 7.92 (s, 1H), 7.88 (d, J=2.2 Hz, 1H), 7.63 (d, J=2.3 Hz, 1H), 6.96 (d, J=1.2 Hz, 1H), 3.89 (t, J=5.6 Hz, 2H), 3.78 (s, 3H), 3.48 (t, J=5.6 Hz, 2H). UPLC-MS (Basic 2 min): rt=0.68 min, m/z (ES+) 297.1, 299.0

### Methyl 8-chloro-5,6-dihydrobenzo[f]imidazo[1,5-d][1,4]oxazepine-10-carboxylate

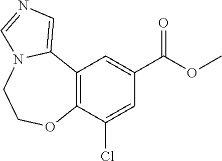 [SMILES: COC(=O)c1cc(Cl)c2c(c1)-c1cncn1CCO2]

Prepared from methyl 3-chloro-4-hydroxy-5-(1-(2-hydroxyethyl)-1H-imidazol-5-yl)benzoate according to General Procedure C to afford the desired product methyl 8-chloro-5,6-dihydrobenzo[f]imidazo[1,5-d][1,4]oxazepine-10-carboxylate (330 mg, 1.14 mmol, 88% yield) as an off-white solid. ¹H NMR (400 MHz, DMSO-D6) δ 8.27 (dd, J=2.1, 0.8 Hz, 1H), 7.90-7.78 (m, 2H), 7.61 (d, J=1.0 Hz, 1H), 4.63-4.57 (m, 2H), 4.56-4.50 (m, 2H), 3.86 (s, 3H). UPLC-MS (Basic 2 min): rt=0.98 min, m/z (ES+) 279.1, 281.1 [M+H]⁺.

### Lithium 8-chloro-5,6-dihydrobenzo[f]imidazo[1,5-d][1,4]oxazepine-10-carboxylate

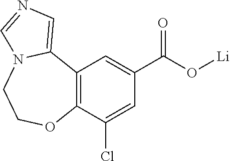

Prepared from methyl 8-chloro-5,6-dihydrobenzo[f]imidazo[1,5-d][1,4]oxazepine-10-carboxylate according to General Procedure D to afford the desired product lithium 8-chloro-5,6-dihydrobenzo[f]imidazo[1,5-d][1,4]oxazepine-10-carboxylate (330 mg, 1.10 mmol, 97% yield) as a purple solid. ¹H NMR (400 MHz, DMSO-D6) δ 8.13 (d, J=1.9 Hz, 1H), 7.78 (d, J=1.1 Hz, 1H), 7.75 (d, J=1.8 Hz, 1H), 7.41 (d, J=1.1 Hz, 1H), 4.52-4.42 (m, 4H). UPLC-MS (Basic 2 min): rt=0.62 min, m/z (ES+) 265.1, 267.0 [M+H]⁺.

### Methyl 2,3-difluoro-5-formyl-4-hydroxybenzoate

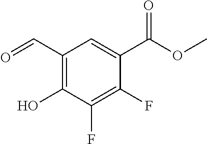

Prepared from methyl 2,3-difluoro-4-hydroxybenzoate according to General Procedure F to afford the desired product methyl 2,3-difluoro-5-formyl-4-hydroxybenzoate (425 mg, 1.69 mmol, 64% yield) as a yellow solid. ¹H NMR (400 MHz, DMSO-D6) δ 10.18 (s, 1H), 8.07-8.0 (m, 1H), 3.82 (s, 3H). 1H not observed. UPLC-MS (Basic 2 min): rt=0.63 min, m/z (ES+) 215.1 [M−H]⁻.

### Methyl (E)-2,3-difluoro-4-hydroxy-5-(((2-hydroxyethyl)imino)methyl)benzoate

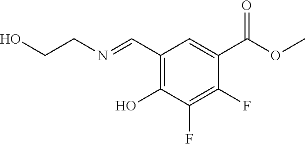 [SMILES: COC(=O)c1cc(/C=N/CCO)c(O)c(F)c1F]

Prepared from ethanolamine and methyl 2,3-difluoro-5-formyl-4-hydroxybenzoate according to General Procedure A to afford the desired product methyl (E)-2,3-difluoro-4-hydroxy-5-(((2-hydroxyethyl)imino)methyl)benzoate (500 mg, 1.93 mmol, 98% yield) as a yellow solid. ¹H NMR (400 MHz, DMSO-D6) δ 8.60 (d, J=1.0 Hz, 1H), 7.86 (dd, J=9.2, 2.0 Hz, 1H), 3.73 (s, 3H), 3.62-3.58 (m, 4H). 2H not observed.

### Methyl 2,3-difluoro-4-hydroxy-5-(1-(2-hydroxyethyl)-1H-imidazol-5-yl)benzoate

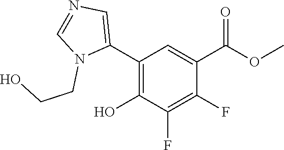 [SMILES: COC(=O)c1cc(-c2cncn2CCO)c(O)c(F)c1F]

Prepared from methyl (E)-2,3-difluoro-4-hydroxy-5-(((2-hydroxyethyl)imino)methyl)benzoate according to General Procedure B to afford the desired product methyl 2,3-difluoro-4-hydroxy-5-(1-(2-hydroxyethyl)-1H-imidazol-5-yl)benzoate (325 mg, 0.719 mmol, 37% yield) as a yellow solid. The crude product was used directly in the next step without further purification. UPLC-MS (Basic 2 min): rt=0.64 min, m/z (ES+) 299.1

### Methyl 8,9-difluoro-5,6-dihydrobenzo[f]imidazo[1,5-d][1,4]oxazepine-10-carboxylate

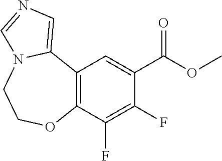 [SMILES: COC(=O)c1cc2c(c(F)c1F)OCCn1cncc1-2]

Prepared from methyl 2,3-difluoro-4-hydroxy-5-(1-(2-hydroxyethyl)-1H-imidazol-5-yl)benzoate according to General Procedure C to afford the desired product methyl 8,9-difluoro-5,6-dihydrobenzo[f]imidazo[1,5-d][1,4]oxazepine-10-carboxylate (110 mg, 0.345 mmol, 32% yield) as an off-white solid. ¹H NMR (400 MHz, DMSO-D6) δ 8.01 (dd, J=7.7, 2.3 Hz, 1H), 7.77 (d, J=1.0 Hz, 1H), 7.53 (d, J=1.1 Hz, 1H), 4.62-4.54 (m, 2H), 4.55-4.48 (m, 2H), 3.84 (s, 3H). UPLC-MS (Basic 2 min): rt=0.92 min, m/z (ES+) 281.0 [M+H]⁺.

### Lithium 8,9-difluoro-5,6-dihydrobenzo[f]imidazo[1,5-d][1,4]oxazepine-10-carboxylate

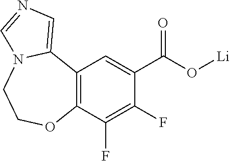

Prepared from methyl 8,9-difluoro-5,6-dihydrobenzo[f]imidazo[1,5-d][1,4]oxazepine-10-carboxylate according to General Procedure D to afford the desired product lithium 8,9-difluoro-5,6-dihydrobenzo[f]imidazo[1,5-d][1,4]oxazepine-10-carboxylate (100 mg, 0.345 mmol, 99% yield) as a grey solid. ¹H NMR (400 MHz, DMSO-D6) δ 7.79-7.69 (m, 2H), 7.33 (d, J=1.1 Hz, 1H), 4.45 (d, J=1.6 Hz, 4H).

### Methyl 5-formyl-4-hydroxy-2,3-dimethylbenzoate

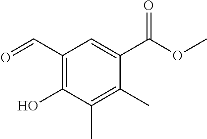

Prepared from methyl 4-hydroxy-2,3-dimethylbenzoate according to General Procedure F to afford the desired product methyl 5-formyl-4-hydroxy-2,3-dimethylbenzoate (711 mg, 3.41 mmol, 62% yield) as an off-white solid. ¹H NMR (400 MHz, DMSO-D6) δ 11.49 (s, 1H), 10.02 (s, 1H), 8.13 (s, 1H), 3.82 (s, 3H), 2.17 (s, 3H). 3H not observed. UPLC-MS (Basic 2 min): rt=0.97 min, m/z (ES+) 207.2 [M−H]⁻.

### Methyl (E)-4-hydroxy-5-(((2-hydroxyethyl)imino)methyl)-2,3-dimethylbenzoate

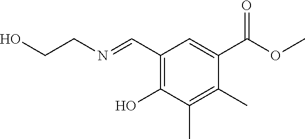 [SMILES: COC(=O)c1cc(/C=N/CCO)c(O)c(C)c1C]

Prepared from ethanolamine and methyl 5-formyl-4-hydroxy-2,3-dimethylbenzoate according to General Procedure A to afford the desired product methyl (E)-4-hydroxy-5-(((2-hydroxyethyl)imino)methyl)-2,3-dimethylbenzoate (955 mg, 3.80 mmol, 99% yield) as a yellow oil. ¹H NMR (400 MHz, DMSO-D6) δ 8.53 (s, 1H), 7.82 (s, 1H), 3.77 (s, 3H), 3.64 (s, 4H), 3.17 (s, 1H), 2.44 (s, 3H), 2.11 (s, 3H). 1 OH not observed.

### Methyl 4-hydroxy-5-(1-(2-hydroxyethyl)-1H-imidazol-5-yl)-2,3-dimethylbenzoate

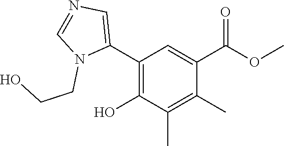 [SMILES: COC(=O)c1cc(-c2cncn2CCO)c(O)c(C)c1C]

Prepared from methyl (E)-4-hydroxy-5-(((2-hydroxyethyl)imino)methyl)-2,3-dimethylbenzoate according to General Procedure B to afford the desired product methyl 4-hydroxy-5-(1-(2-hydroxyethyl)-1H-imidazol-5-yl)-2,3-dimethylbenzoate (1.06 g, 3.22 mmol, 85% yield) as an off-white solid. ¹H NMR (400 MHz, DMSO-D6) δ 7.73 (s, 1H), 7.45 (s, 1H), 6.84 (s, 1H), 3.82 (t, J=5.9 Hz, 2H), 3.77 (s, 3H), 3.47 (t, J=5.9 Hz, 2H), 2.46 (s, 3H), 2.20 (s, 3H). 2 OH not observed. UPLC-MS (Basic 2 min): rt=0.79 min, m/z (ES+) 291.3 [M+H]⁺.

### Methyl 8,9-dimethyl-5,6-dihydrobenzo[f]imidazo[1,5-d][1,4]oxazepine-10-carboxylate

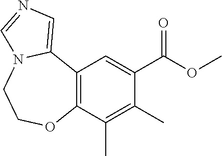 [SMILES: COC(=O)c1cc2c(c(C)c1C)OCCn1cncc1-2]

Triphenylphosphine (1.44 g, 5.48 mmol) was dissolved in diethyl ether (20 mL) and the resultant solution was stirred and cooled to 0° C. Diisopropyl azodicarboxylate (1.1 mL, 5.59 mmol) was added, which caused the formation of a dense precipitate. After 15 min., methyl 4-hydroxy-5-(1-(2-hydroxyethyl)-1H-imidazol-5-yl)-2,3-dimethylbenzoate (1.06 g, 3.65 mmol) was added, and the reaction mixture was allowed to warm to room temperature. After 4 h, the reaction mixture was filtered, and the insoluble material was washed with Et₂O. The combined filtrates were concentrated under reduced pressure and the residue was purified by reverse phase column chromatography over C18 eluting with a gradient of MeCN(0.1% NH₃) (5% to 95%; v/v) in water (0.1% NH₃) to afford the desired product methyl 8,9-dimethyl-5,6-dihydrobenzo[f]imidazo[1,5-d][1,4]oxazepine-10-carboxylate (1.50 g, 3.57 mmol, 98% yield) as an off-white solid. ¹H NMR (400 MHz, DMSO-D6) δ 7.96 (s, 1H), 7.77 (s, 1H), 7.42 (s, 1H), 4.50-4.40 (m, 4H), 3.82 (s, 3H), 2.38 (s, 3H), 2.20 (s, 3H). UPLC-MS (Basic 2 min): rt=1.00 min, m/z (ES+) 273.2 [M+H]+E.

### Lithium 8,9-dimethyl-5,6-dihydrobenzo[f]imidazo[1,5-d][1,4]oxazepine-10-carboxylate

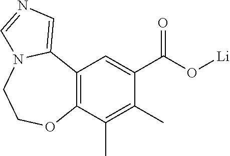

Prepared from methyl 8,9-dimethyl-5,6-dihydrobenzo[f]imidazo[1,5-d][1,4]oxazepine-10-carboxylate according to General Procedure D, the crude solid was diluted with water (30 mL) and washed with diethyl ether (2×50 mL) and DCM (2×50 mL). The aqueous phase was concentrated under reduced pressure to afford the desired product lithium 8,9-dimethyl-5,6-dihydrobenzo[f]imidazo[1,5-d][1,4]oxazepine-10-carboxylate (566 mg, 2.14 mmol, 60% yield) as a yellow solid. ¹H NMR (400 MHz, DMSO-D6) δ 7.71 (d, J=1.1 Hz, 1H), 7.57 (d, J=8.9 Hz, 1H), 7.22 (d, J=1.1 Hz, 1H), 4.40-4.31 (m, 4H), 2.35 (s, 3H), 2.12 (s, 3H). UPLC-MS (Basic 2 min): rt=0.69 min, m/z (ES+) 259.3 [M+H]⁺.

### Methyl 4-(benzyloxy)-3-chloro-2-methylbenzoate

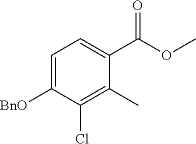 [SMILES: COC(=O)c1ccc(OCc2ccccc2)c(Cl)c1C]

3-Chloro-2-methyl-4-phenylmethoxybenzoic acid (864 mg, 3.12 mmol) and sulfuric acid (0.17 mL, 3.19 mmol) in methanol (20 mL) was heated at reflux for 19 h. The reaction mixture was cooled, which caused the formation of a white precipitate. This was collected by filtration, washed with MeOH and dried under reduced pressure to afford the desired product methyl 4-(benzyloxy)-3-chloro-2-methylbenzoate (657 mg, 2.23 mmol, 71% yield) as a white solid. The filtrate was concentrated to dryness under reduced pressure and the crude residue was purified by column chromatography over C18 eluting with a gradient of MeCN(0.1% NH₃) (5% to 95%; v/v) in water (0.1% NH₃) to afford the desired product methyl 4-(benzyloxy)-3-chloro-2-methylbenzoate (166 mg, 0.546 mmol, 17% yield) as a white solid. ¹H NMR (400 MHz, DMSO-D6) δ 7.78 (d, J=8.8 Hz, 1H), 7.51-7.45 (m, 2H), 7.42 (ddd, J=7.5, 6.7, 1.4 Hz, 2H), 7.38-7.31 (m, 1H), 7.19 (d, J=8.8 Hz, 1H), 5.28 (s, 2H), 3.80 (s, 3H), 2.57 (s, 3H). UPLC-MS (Basic 2 min): rt=1.32 min, m/z not observed.

### Methyl 3-chloro-4-hydroxy-2-methylbenzoate

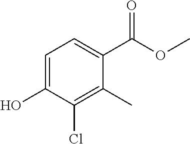

Methyl 4-(benzyloxy)-3-chloro-2-methylbenzoate (690 mg, 2.34 mmol) and 10% palladium on activated carbon (498 mg, 0.234 mmol) were stirred in IPA (14 mL) under a balloon of H₂ at rt for 1 h. The reaction mixture was filtered through Celite, which was then washed with IPA, DCM and MeOH. The combined filtrates were concentrated under reduced pressure to afford the desired product methyl 3-chloro-4-hydroxy-2-methylbenzoate (456 mg, 2.27 mmol, 97% yield) as an off-white solid. ¹H NMR (400 MHz, DMSO-D6) δ 10.94 (s, 1H), 7.65 (dd, J=8.7, 1.2 Hz, 1H), 6.90 (d, J=8.7 Hz, 1H), 3.77 (s, 3H), 2.55 (s, 3H). UPLC-MS (Basic 2 min): rt=0.75 min, m/z not observed.

### Methyl 3-chloro-5-formyl-4-hydroxy-2-methylbenzoate

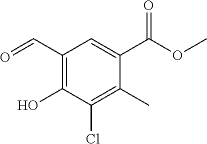

Hexamethylenetetramine (671 mg, 4.79 mmol) was added to a solution of methyl 3-chloro-4-hydroxy-2-methylbenzoate (480 mg, 2.39 mmol) in trifluoroacetic acid (5.0 mL, 65.3 mmol) at rt and then heated at 75° C. for 18 h. Water (30 mL) was added and the reaction stirred for 1 h. This was extracted with EtOAc (2×50 mL). The combined organic layers were dried (Na₂SO₄), filtered and concentrated under reduced pressure to give crude material. This was triturated with diethyl ether (30 mL) and the resulting solid collected and dried under reduced pressure to afford the desired product methyl 3-chloro-5-formyl-4-hydroxy-2-methylbenzoate (280 mg, 1.04 mmol, 44% yield) as a yellow solid. ¹H NMR (400 MHz, DMSO-D6) δ 11.63 (s, 1H), 10.15 (s, 1H), 8.22 (s, 1H), 3.84 (s, 3H), 2.65 (s, 3H). UPLC-MS (Basic 2 min): rt=0.70 min, m/z (ES+) 227.1, 229.1 [M+H]⁺.

### Methyl (E)-3-chloro-4-hydroxy-5-(((2-hydroxyethyl)imino)methyl)-2-methylbenzoate

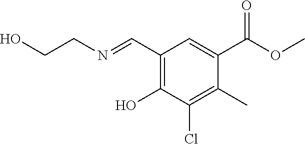 [SMILES: COC(=O)c1cc(/C=N/CCO)c(O)c(Cl)c1C]

Prepared from ethanolamine and methyl 3-chloro-5-formyl-4-hydroxy-2-methylbenzoate according to General Procedure A to afford the desired product methyl (E)-3-chloro-4-hydroxy-5-(((2-hydroxyethyl)imino)methyl)-2-methylbenzoate (350 mg, 1.03 mmol, 99% yield) as an orange solid. ¹H NMR (400 MHz, DMSO-D6) δ 8.61 (s, 1H), 7.96 (s, 1H), 5.11 (s, 1H), 3.75 (s, 3H), 3.65 (dt, J=7.7, 4.2 Hz, 4H), 2.57 (s, 3H). 1 OH not observed.

### Methyl 3-chloro-4-hydroxy-5-(1-(2-hydroxyethyl)-1H-imidazol-5-yl)-2-methylbenzoate

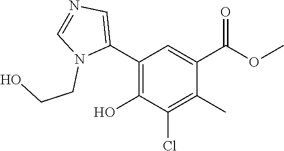 [SMILES: COC(=O)c1cc(-c2cncn2CCO)c(O)c(Cl)c1C]

Prepared from methyl (E)-3-chloro-4-hydroxy-5-(((2-hydroxyethyl)imino)methyl)-2-methylbenzoate according to General Procedure B to afford the desired product methyl 3-chloro-4-hydroxy-5-(1-(2-hydroxyethyl)-1H-imidazol-5-yl)-2-methylbenzoate (360 mg, 1.01 mmol, 98% yield) as a yellow solid. ¹H NMR (400 MHz, DMSO-D6) δ 7.69 (s, 1H), 7.55 (s, 1H), 6.73 (s, 1H), 3.85 (t, J=5.6 Hz, 2H), 3.71 (s, 3H), 3.58-3.54 (m, 1H), 3.51 (t, J=5.7 Hz, 2H), 2.88-2.81 (m, 1H), 2.57 (s, 3H). UPLC-MS (Basic 2 min): rt=0.69 min, m/z (ES+) 311.0, 313.0

### Methyl 8-chloro-9-methyl-5,6-dihydrobenzo[f]imidazo[1,5-d][1,4]oxazepine-10-carboxylate

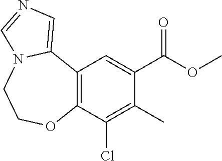 [SMILES: COC(=O)c1cc2c(c(Cl)c1C)OCCn1cncc1-2]

Prepared from methyl 3-chloro-4-hydroxy-5-(1-(2-hydroxyethyl)-1H-imidazol-5-yl)-2-methylbenzoate according to General Procedure C to afford the desired product methyl 8-chloro-9-methyl-5,6-dihydrobenzo[f]imidazo[1,5-d][1,4]oxazepine-10-carboxylate (240 mg, 0.738 mmol, 73% yield) as a yellow solid. ¹H NMR (400 MHz, DMSO-D6) δ 8.13 (s, 1H), 7.81 (d, J=1.1 Hz, 1H), 7.55 (d, J=1.1 Hz, 1H), 4.60-4.54 (m, 2H), 4.54-4.47 (m, 2H), 3.85 (s, 3H), 2.53 (s, 3H). UPLC-MS (Basic 2 min): rt=1.04 min, m/z (ES+) 293.1, 295.1 [M+H]⁺.

### Lithium 8-chloro-9-methyl-5,6-dihydrobenzo[f]imidazo[1,5-d][1,4]oxazepine-10-carboxylate

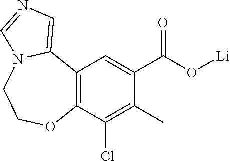

Prepared from methyl 8-chloro-9-methyl-5,6-dihydrobenzo[f]imidazo[1,5-d][1,4]oxazepine-10-carboxylate according to General Procedure D to afford the desired product lithium 8-chloro-9-methyl-5,6-dihydrobenzo[f]imidazo[1,5-d][1,4]oxazepine-10-carboxylate (242 mg, 0.689 mmol, 93% yield) as a blue solid. ¹H NMR (400 MHz, DMSO-D6) δ 7.75 (d, J=1.1 Hz, 1H), 7.71 (s, 1H), 7.33 (d, J=1.1 Hz, 1H), 4.48-4.38 (m, 4H), 2.46 (s, 3H). UPLC-MS (Basic 2 min): rt=0.63 min, m/z (ES+) 279.1, 281.1 [M+H]⁺.

### (1r,4r)—N,N-Dibenzyl-4-(3-(difluoromethyl)azetidin-1-yl)cyclohexan-1-amine

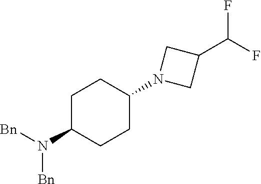 [SMILES: FC(F)C1CN([C@H]2CC[C@H](N(Cc3ccccc3)Cc3ccccc3)CC2)C1]

Prepared from 4-(dibenzylamino)cyclohexanone and 3-(difluoromethyl)azetidine hydrochloride according to General Procedure G to afford the desired product (1r,4r)—N,N-dibenzyl-4-(3-(difluoromethyl)azetidin-1-yl)cyclohexan-1-amine (300 mg, 0.421 mmol, 21% yield) as a colourless oil. UPLC-MS (Basic 2 min): rt=1.45 min, m/z (ES+) 385.3 [M+H]⁺.

### (1r,4r)-4-(3-(Difluoromethyl)azetidin-1-yl)cyclohexan-1-amine

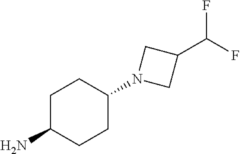 [SMILES: N[C@H]1CC[C@H](N2CC(C(F)F)C2)CC1]

Prepared from (1r,4r)—N,N-dibenzyl-4-(3-(difluoromethyl)azetidin-1-yl)cyclohexan-1-amine according to General Procedure H to afford the desired product (1r,4r)-4-(3-(difluoromethyl)azetidin-1-yl)cyclohexan-1-amine (110 mg, 0.404 mmol, 98% yield) as a colourless oil. UPLC-MS (Basic 2 min): rt=0.69 min, m/z (ES+) 205.2 [M+H]⁺.

### (1r,4r)—N1,N1-Dibenzyl-N4-(2-(methylsulfonyl)ethyl)cyclohexane-1,4-diamine

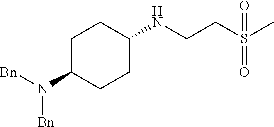 [SMILES: CS(=O)(=O)CCN[C@H]1CC[C@H](N(Cc2ccccc2)Cc2ccccc2)CC1]

Prepared from 4-(dibenzylamino)cyclohexanone and 2-(methylsulfonyl)ethylamine hydrochloride according to General Procedure G to afford the desired product (1r,4r)—N1,N1-dibenzyl-N4-(2-(methylsulfonyl)ethyl)cyclohexane-1,4-diamine (120 mg, 0.297 mmol, 17% yield) as a white solid. UPLC-MS (Basic 2 min): rt=1.20 min, m/z (ES+) 401.5 [M+H]⁺.

### (1r,4r)-N1-(2-(Methylsulfonyl)ethyl)cyclohexane-1,4-diamine

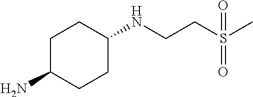

Prepared from (1r,4r)—N1,N1-dibenzyl-N4-(2-(methylsulfonyl)ethyl)cyclohexane-1,4-diamine according to General Procedure H to afford the desired product (1r,4r)-N1-(2-(methylsulfonyl)ethyl)cyclohexane-1,4-diamine (63 mg, 0.249 mmol, 83% yield) as a white solid. ¹H NMR (400 MHz, DMSO-D6) δ 3.12 (m, 4H), 2.93-2.82 (m, 2H), 2.28-2.20 (m, 1H), 1.81-1.69 (m, 4H), 1.06-0.86 (m, 4H). 5H not observed.

### (1r,4r)—N1,N1-Dibenzyl-N4-(2-fluoro-2-methylpropyl)cyclohexane-1,4-diamine

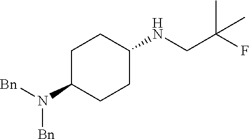 [SMILES: CC(C)(F)CN[C@H]1CC[C@H](N(Cc2ccccc2)Cc2ccccc2)CC1]

Prepared from 4-(dibenzylamino)cyclohexanone and 2-fluoro-2-methylpropan-1-amine hydrochloride according to General Procedure G to afford the desired product (1r,4r)—N1,N1-dibenzyl-N4-(2-fluoro-2-methylpropyl)cyclohexane-1,4-diamine (209 mg, 0.549 mmol, 32% yield) as a white solid. ¹H NMR (400 MHz, DMSO-D6) δ 7.38-7.24 (m, 8H), 7.23-7.10 (m, 2H), 3.56 (s, 4H), 2.58 (d, J=18.6 Hz, 1H), 2.32 (d, J=44.6 Hz, 2H), 1.96-1.68 (m, 4H), 1.46-1.31 (m, 2H), 1.28 (s, 3H), 1.23 (s, 3H), 0.91-0.74 (m, 4H). 2H not observed. UPLC-MS (Basic 4 min): rt=2.63 min, m/z (ES+) 369.4 [M+H]⁺.

### (1r,4r)-N1-(2-Fluoro-2-methylpropyl)cyclohexane-1,4-diamine

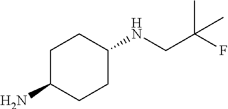

Prepared from (1r,4r)—N1,N1-dibenzyl-N4-(2-fluoro-2-methylpropyl)cyclohexane-1,4-diamine according to General Procedure H to afford the desired product (1r,4r)-N1-(2-fluoro-2-methylpropyl)cyclohexane-1,4-diamine (62 mg, 0.329 mmol, 77% yield) as an off-white solid. ¹H NMR (400 MHz, DMSO-D6) δ 3.32 (s, 3H), 2.67-2.55 (m, 2H), 2.48-2.41 (m, 1H), 2.32-2.18 (m, 1H), 1.98-1.49 (m, 4H), 1.28 (s, 6H), 1.07-0.81 (m, 4H).

### (1r,4r)—N1,N1-Dibenzyl-N4-(2,2-difluorocyclobutyl)cyclohexane-1,4-diamine

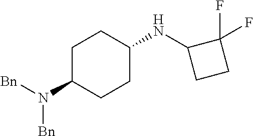 [SMILES: FC1(F)CCC1N[C@H]1CC[C@H](N(Cc2ccccc2)Cc2ccccc2)CC1]

Prepared from 4-(dibenzylamino)cyclohexanone and 2,2-difluorocyclobutan-1-amine hydrochloride according to General Procedure G to afford the desired product (1r,4r)—N1,N1-dibenzyl-N4-(2,2-difluorocyclobutyl)cyclohexane-1,4-diamine (90 mg, 0.185 mmol, 29% yield) as a white solid. ¹H NMR (400 MHz, DMSO-D6) δ 7.38-7.25 (m, 8H), 7.23-7.09 (m, 2H), 3.55 (s, 4H), 3.53-3.48 (m, 1H), 2.45-2.23 (m, 2H), 2.21-2.04 (m, 2H), 2.04-1.70 (m, 5H), 1.46-1.20 (m, 3H), 0.95-0.76 (m, 2H). 1H not observed. UPLC-MS (Basic 4 min): rt=2.53 min, m/z (ES+) 385.4 [M+H]⁺.

### (1r,4r)-N1-(2,2-Difluorocyclobutyl)cyclohexane-1,4-diamine

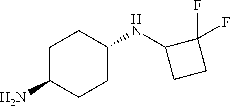 [SMILES: N[C@H]1CC[C@H](NC2CCC2(F)F)CC1]

Prepared from (1r,4r)—N1,N1-dibenzyl-N4-(2,2-difluorocyclobutyl)cyclohexane-1,4-diamine according to General Procedure H to afford the desired product (1r,4r)-N1-(2,2-difluorocyclobutyl)cyclohexane-1,4-diamine (36 mg, 0.174 mmol, 94% yield) as an off-white solid. ¹H NMR (400 MHz, DMSO-D6) δ 3.72-3.48 (m, 1H), 2.59-2.53 (m, 1H), 2.40-2.27 (m, 1H), 2.22-1.92 (m, 3H), 1.78 (dd, J=24.9, 13.2 Hz, 4H), 1.42-1.28 (m, 1H), 1.08-0.93 (m, 5H). 2H not observed.

### N1,N1-Dibenzyl-N4-(3,3,3-trifluoropropyl)cyclohexane-1,4-diamine

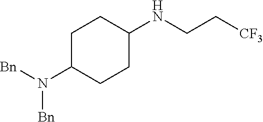 [SMILES: FC(F)(F)CCNC1CCC(N(Cc2ccccc2)Cc2ccccc2)CC1]

Prepared from 4-(dibenzylamino)cyclohexanone and 3,3,3-trifluoropropan-1-amine hydrochloride according to General Procedure G, using MeCN as the solvent, to afford the desired product N1,N1-dibenzyl-N4-(3,3,3-trifluoropropyl)cyclohexane-1,4-diamine (259 mg, 0.663 mmol, 65% yield) as a colourless oil. ¹H NMR (400 MHz, DMSO-D6) δ 7.34 (ddd, J=8.2, 2.4, 1.4 Hz, 4H), 7.31-7.25 (m, 4H), 7.23-7.14 (m, 2H), 3.57 (d, J=10.7 Hz, 4H), 2.67 (dt, J=12.5, 7.3 Hz, 2H), 2.45-2.25 (m, 3H), 1.88 (d, J=12.6 Hz, 1H), 1.79 (d, J=12.3 Hz, 1H), 1.75-1.54 (m, 3H), 1.54-1.46 (m, 1H), 1.39 (dt, J=14.0, 11.1 Hz, 1H), 1.20 (td, J=11.8, 7.2 Hz, 1H), 0.88-0.75 (m, 1H). 1H not observed. UPLC-MS (Basic 4 min): rt=2.55 min, m/z (ES+) 391.4 [M+H]⁺, rt=2.64 min, m/z (ES+) 391.4 [M+H]⁺.

### N1-(3,3,3-Trifluoropropyl)cyclohexane-1,4-diamine

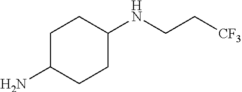 [SMILES: NC1CCC(NCCC(F)(F)F)CC1]

Prepared from N1,N1-dibenzyl-N4-(3,3,3-trifluoropropyl)cyclohexane-1,4-diamine according to General Procedure H to afford the desired product N1-(3,3,3-trifluoropropyl)cyclohexane-1,4-diamine (100 mg, 0.476 mmol, 72% yield) as a brown oil. 1H NMR (400 MHz, DMSO-D6) δ 2.77-2.58 (m, 3H), 2.44-2.19 (m, 3H), 1.87-1.36 (m, 9H), 1.03-0.90 (m, 2H).

### tert-Butyl ((1r,4r)-4-((3,3-difluoroazetidin-1-yl)methyl)cyclohexyl)carbamate

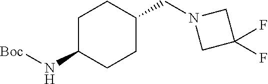 [SMILES: CC(C)(C)OC(=O)N[C@H]1CC[C@H](CN2CC(F)(F)C2)CC1]

Acetic acid (0.13 mL, 2.31 mmol) was added to a solution of tert-butyl N-(4-formylcyclohexyl)carbamate (500 mg, 2.20 mmol) and 3,3-difluoroazetidine hydrochloride (342 mg, 2.64 mmol) in MeCN(10 mL) at 25° C. and stirred for 4 h. Sodium triacetoxy borohydride (932 mg, 4.40 mmol) was added portionwise and the reaction stirred for 68 h. This was quenched with methanol (10 mL) and concentrated under reduced pressure to give a residue, which was dissolved in EtOAc (25 mL). The resulting solution was washed with sat. aq. NaHCO₃ (25 mL). This was extracted with EtOAc (25 mL). The combined organic layers were dried (Na₂SO₄), filtered and concentrated under reduced pressure to give crude product. This was purified by column chromatography over silica eluting with a gradient of EtOAc (0% to 60%; v/v) in iso-hexane to afford the desired product tert-butyl ((1r,4r)-4-((3,3-difluoroazetidin-1-yl)methyl)cyclohexyl)carbamate (395 mg, 1.17 mmol, 53% yield) as a white solid. ¹H NMR (400 MHz, DMSO-D6) δ 6.69 (d, J=8.1 Hz, 1H), 3.51 (td, J=12.5, 1.7 Hz, 4H), 3.19-3.03 (m, 1H), 2.41-2.36 (m, 1H), 2.36-2.29 (m, 2H), 1.77-1.67 (m, 4H), 1.44-1.34 (m, 9H), 1.16-1.00 (m, 2H), 0.94-0.77 (m, 2H).

### (1r,4r)-4-((3,3-Difluoroazetidin-1-yl)methyl)cyclohexan-1-amine di-2,2,2-trifluouroacetate

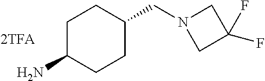 [SMILES: N[C@H]1CC[C@H](CN2CC(F)(F)C2)CC1]

Trifluoroacetic acid (0.36 mL, 4.67 mmol) was added to a solution of tert-butyl ((1r,4r)-4-((3,3-difluoroazetidin-1-yl)methyl)cyclohexyl)carbamate (395 mg, 1.17 mmol) in DCM (10 mL) at 25° C. and stirred for 18 h. This was concentrated under reduced pressure and re-dissolved in DCM (10 mL) and trifluoroacetic acid (0.36 mL, 4.67 mmol) was added and heated at reflux for 20 h. This was concentrated under reduced pressure to yield (1r,4r)-4-((3,3-difluoroazetidin-1-yl)methyl)cyclohexan-1-amine di-2,2,2-trifluouroacetate (680 mg, 0.786 mmol, 67% yield) as a dense white oil. Used directly in the next step without further characterisation.

### 1-(3-Fluoropropoxy)-4-nitrobenzene

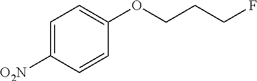 [SMILES: O=[N+]([O-])c1ccc(OCCCF)cc1]

Prepared from 3-fluoropropan-1-ol and 1-fluoro-4-nitrobenzene according to General Procedure I to afford the desired product 1-(3-fluoropropoxy)-4-nitrobenzene (610 mg, 3.01 mmol, 85% yield) as a yellow oil. ¹H NMR (400 MHz, DMSO-D6) δ 8.25-8.16 (m, 2H), 7.21-7.12 (m, 2H), 4.62 (dt, J=47.2, 5.9 Hz, 2H), 4.23 (t, J=6.3 Hz, 2H), 2.15 (dp, J=25.8, 6.1 Hz, 2H).

### 4-(3-Fluoropropoxy)cyclohexan-1-amine

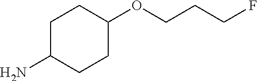 [SMILES: NC1CCC(OCCCF)CC1]

Prepared from 1-(3-fluoropropoxy)-4-nitrobenzene according to General Procedure J to afford the desired product 4-(3-fluoropropoxy)cyclohexan-1-amine (353 mg, 2.01 mmol, 66% yield) as a brown oil. ¹H NMR (400 MHz, DMSO-D6) δ 4.66-4.32 (m, 2H), 3.56-3.27 (m, 3H), 3.22-2.54 (m, 1H), 1.92-1.68 (m, 4H), 1.56-1.20 (m, 6H), 1.19-0.96 (m, 1H), 0.93 (dd, J=6.2, 3.0 Hz, 1H).

### 1-Nitro-4-((1-(trifluoromethyl)cyclopropyl)methoxy)benzene

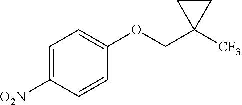 [SMILES: O=[N+]([O-])c1ccc(OCC2(C(F)(F)F)CC2)cc1]

Prepared from 1-(trifluoromethyl)cyclopropanemethanol and 1-fluoro-4-nitrobenzene according to General Procedure I to afford the desired product 1-nitro-4-((1-(trifluoromethyl)cyclopropyl)methoxy)benzene (650 mg, 2.49 mmol, 88% yield) as an off-white solid. ¹H NMR (400 MHz, DMSO-D6) δ 8.26-8.16 (m, 2H), 7.21-7.12 (m, 2H), 4.29 (s, 2H), 1.17-1.00 (m, 4H).

### 4-((1-(Trifluoromethyl)cyclopropyl)methoxy)cyclohexan-1-amine

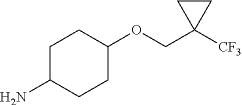 [SMILES: NC1CCC(OCC2(C(F)(F)F)CC2)CC1]

Prepared from 1-nitro-4-((1-(trifluoromethyl)cyclopropyl)methoxy)benzene according to General Procedure K to afford the desired product 4-((1-(trifluoromethyl)cyclopropyl)methoxy)cyclohexan-1-amine (382 mg, 1.35 mmol, 54% yield) as a colourless gum. ¹H NMR (400 MHz, DMSO-D6) δ 3.52-3.08 (m, 3H), 2.50-2.31 (m, 1H), 1.93-0.65 (m, 12H). 2H not observed. UPLC-MS (Basic 2 min): rt=0.86 min, m/z (ES+) 238.2 [M+H]⁺.

### 1-Nitro-4-(3,3,3-trifluoro-2,2-dimethylpropoxy)benzene

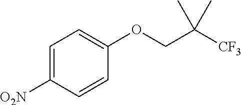 [SMILES: CC(C)(COc1ccc([N+](=O)[O-])cc1)C(F)(F)F]

Prepared from 3,3,3-trifluoro-2,2, -dimethylpropan-1-ol and 1-fluoro-4-nitrobenzene according to General Procedure I to afford the desired product 1-nitro-4-(3,3,3-trifluoro-2,2-dimethylpropoxy)benzene (595 mg, 2.26 mmol, 80% yield) as a yellow oil. ¹H NMR (400 MHz, DMSO-D6) δ 8.25-8.18 (m, 2H), 7.23-7.17 (m, 2H), 4.15 (s, 2H), 1.24 (s, 6H).

### 4-(3,3,3-Trifluoro-2,2-dimethylpropoxy)cyclohexan-1-amine

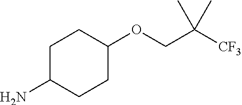 [SMILES: CC(C)(COC1CCC(N)CC1)C(F)(F)F]

Prepared from 1-nitro-4-(3,3,3-trifluoro-2,2-dimethylpropoxy)benzene according to General Procedure K to afford the desired product 4-(3,3,3-trifluoro-2,2-dimethylpropoxy)cyclohexan-1-amine (202 mg, 0.843 mmol, 37% yield) as a colourless gum. ¹H NMR (400 MHz, DMSO-D6) δ 3.47-3.02 (m, 3H), 2.49-2.36 (m, 1H), 1.93-0.83 (m, 14H). 2H not observed. UPLC-MS (Basic 2 min): rt=1.05 min, m/z (ES+) 240.2 [M+H]⁺.

### 2-((4-Nitrophenoxy)methyl)tetrahydrofuran

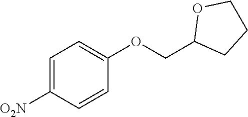 [SMILES: O=[N+]([O-])c1ccc(OCC2CCCO2)cc1]

Prepared from tetrahydrofurfuryl alcohol and 1-fluoro-4-nitrobenzene according to General Procedure I to afford the desired product 2-((4-nitrophenoxy)methyl)tetrahydrofuran (637 mg, 2.85 mmol, 80% yield) as a brown solid. ¹H NMR (400 MHz, DMSO-D6) δ 8.24-8.16 (m, 2H), 7.20-7.12 (m, 2H), 4.23-4.11 (m, 2H), 4.06 (dd, J=10.2, 6.4 Hz, 1H), 3.78 (dt, J=8.1, 6.6 Hz, 1H), 3.68 (td, J=7.7, 6.2 Hz, 1H), 2.01 (dddd, J=12.2, 8.5, 7.0, 5.3 Hz, 1H), 1.95-1.77 (m, 2H), 1.67 (ddt, J=12.0, 8.5, 6.8 Hz, 1H).

### 4-((Tetrahydrofuran-2-yl)methoxy)cyclohexan-1-amine

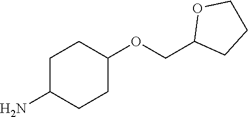 [SMILES: NC1CCC(OCC2CCCO2)CC1]

Prepared from 2-((4-nitrophenoxy)methyl)tetrahydrofuran according to General Procedure J to afford the desired product 4-((tetrahydrofuran-2-yl)methoxy)cyclohexan-1-amine (411 mg, 2.06 mmol, 72% yield) as a brown oil. ¹H NMR (400 MHz, DMSO-D6) δ 3.95-3.80 (m, 1H), 3.77-3.66 (m, 1H), 3.66-3.54 (m, 1H), 3.42-3.24 (m, 3H), 2.89-2.52 (m, 1H), 1.92-1.67 (m, 5H), 1.59-1.23 (m, 7H), 1.17-0.92 (m, 2H).

### 1-(3,3-Difluorocyclobutoxy)-4-nitrobenzene

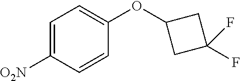 [SMILES: O=[N+]([O-])c1ccc(OC2CC(F)(F)C2)cc1]

1-Fluoro-4-nitrobenzene (400 mg, 2.83 mmol), 3,3-difluorocyclobutanol (614 mg, 5.68 mmol) and potassium hydroxide (318 mg, 5.67 mmol) were suspended in MeCN(6 mL) and stirred at rt for 19 h. This was diluted with H₂O, then purified directly by column chromatography over C18 eluting with a gradient of MeCN(0.1% NH₃) (5% to 95%; v/v) in water (0.1% NH₃) to afford the desired product 1-(3,3-difluorocyclobutoxy)-4-nitrobenzene (600 mg, 2.62 mmol, 92% yield) as a yellow solid. ¹H NMR (400 MHz, DMSO-D6) δ 8.25-8.17 (m, 2H), 7.17-7.09 (m, 2H), 4.93 (dddd, J=13.5, 12.0, 5.8, 3.6 Hz, 1H), 3.31-3.19 (m, 2H), 2.86-2.67 (m, 2H).

### 4-(3,3-Difluorocyclobutoxy)cyclohexan-1-amine

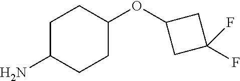 [SMILES: NC1CCC(OC2CC(F)(F)C2)CC1]

Prepared from 1-(3,3-difluorocyclobutoxy)-4-nitrobenzene according to General Procedure K to afford the desired product 4-(3,3-difluorocyclobutoxy)cyclohexan-1-amine (258 mg, 1.26 mmol, 48% yield) as a yellow oil. ¹H NMR (400 MHz, DMSO-D6) δ 4.09-3.93 (m, 2H), 3.52-3.11 (m, 1H), 2.81 (tq, J=17.8, 6.1 Hz, 2H), 2.48-2.28 (m, 2H), 2.11-0.77 (m, 8H). 2H not observed.

### 1-(3-Fluorocyclobutoxy)-4-nitrobenzene

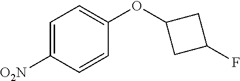 [SMILES: O=[N+]([O-])c1ccc(OC2CC(F)C2)cc1]

1-Fluoro-4-nitrobenzene (400 mg, 2.83 mmol), 3-fluorocyclobutanol (511 mg, 5.67 mmol) and potassium hydroxide (318 mg, 5.67 mmol) were suspended in MeCN(8 mL) and stirred at rt for 5 h. This was diluted with H₂O, then purified directly by column chromatography over C18 eluting with a gradient of MeCN(0.1% NH₃) (5% to 95%; v/v) in water (0.1% NH₃) to afford the desired product 1-(3-fluorocyclobutoxy)-4-nitrobenzene (554 mg, 2.62 mmol, 92% yield) as a yellow oil. ¹H NMR (400 MHz, DMSO-D6) δ 8.25-8.15 (m, 2H), 7.16-7.01 (m, 2H), 5.33 (dtt, J=56.4, 6.7, 3.9 Hz, 1H), 5.18-5.06 (m, 1H), 2.83-2.66 (m, 2H), 2.59-2.50 (m, 2H).

### 4-(3-Fluorocyclobutoxy)cyclohexan-1-amine

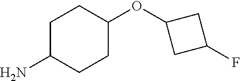 [SMILES: NC1CCC(OC2CC(F)C2)CC1]

Prepared from 1-(3-fluorocyclobutoxy)-4-nitrobenzene according to General Procedure K to afford the desired product 4-(3-fluorocyclobutoxy)cyclohexan-1-amine (216 mg, 1.15 mmol, 44% yield) as a brown oil. UPLC-MS (Basic 2 min): rt=0.79 min, m/z (ES+) 188.2 [M+H]⁺, rt=0.82 min, m/z (ES+) 188.2 [M+H]⁺.

### 2-Methyl-4-(2,2,2-trifluoroethyl)cyclohexan-1-amine

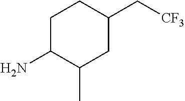

2-methyl-1-nitro-4-(2,2,2-trifluoroethyl)benzene (500 mg, 2.28 mmol) and Nishimura's catalyst (549 mg, 1.14 mmol) were combined in IPA (10 mL) in an ASynt pressure vessel and the resultant suspension was stirred at room temperature. The vessel was then pressurised to 10 bar with H₂ and heated to 90° C. for 43 h. The vessel was allowed to cool, then it was de-pressurised. The reaction mixture was filtered through a Celite cartridge, which was washed with IPA, DCM, MeOH and MeCN. The combined filtrates were carefully evaporated to afford the desired product 2-methyl-4-(2,2,2-trifluoroethyl)cyclohexan-1-amine (190 mg, 0.973 mmol, 43% yield) as a colourless oil. ¹H NMR (400 MHz, D₂O) δ 3.42-2.74 (m, 1H), 2.44-1.86 (m, 3H), 1.86-1.21 (m, 6H), 1.09-0.79 (m, 4H). 2H not observed. UPLC-MS (Basic 2 min): rt=0.96 min, m/z (ES+) 196.2 [M+H]⁺.

### 2-(3-Fluoropropoxy)-5-nitropyrimidine

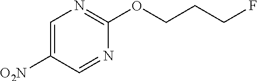 [SMILES: O=[N+]([O-])c1cnc(OCCCF)nc1]

Prepared from 2-chloro-5-nitropyrimidine and 3-fluoropropan-1-ol according to General Procedure L to afford the desired product 2-(3-fluoropropoxy)-5-nitropyrimidine (117 mg, 0.378 mmol, 20% yield) as a brown solid. ¹H NMR (400 MHz, DMSO-D6) δ 9.40 (s, 2H), 4.77-4.46 (m, 4H), 2.18 (dp, J=26.1, 6.2 Hz, 2H).

### 2-(3-Fluoropropoxy)pyrimidin-5-amine

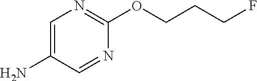 [SMILES: Nc1cnc(OCCCF)nc1]

Prepared from 2-(3-fluoropropoxy)-5-nitropyrimidine according to General Procedure M to afford the desired product 2-(3-fluoropropoxy)pyrimidin-5-amine (107 mg, 0.608 mmol, 74% yield) as a yellow solid. ¹H NMR (400 MHz, DMSO-D6) δ 7.95 (s, 2H), 4.98 (s, 2H), 4.57 (dt, J=47.3, 5.9 Hz, 2H), 4.22 (t, J=6.4 Hz, 2H), 2.06 (dp, J=25.8, 6.2 Hz, 2H). UPLC-MS (Basic 2 min): rt=0.63 min, m/z (ES+) 172.2 [M+H]⁺.

### 2-(3,3-Difluoropropoxy)-5-nitropyrimidine

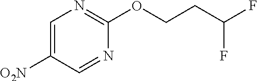 [SMILES: O=[N+]([O-])c1cnc(OCCC(F)F)nc1]

Prepared from 2-chloro-5-nitropyrimidine and 3,3-difluoro-1-propanol according to General Procedure L to afford the desired product 2-(3,3-difluoropropoxy)-5-nitropyrimidine (125 mg, 0.571 mmol, 30% yield) as a brown oil. ¹H NMR (400 MHz, DMSO-D6) δ 9.41 (s, 1H), 9.02 (s, 1H), 6.27 (tt, J=56.1, 4.5 Hz, 1H), 4.61 (t, J=6.2 Hz, 2H), 2.41 (ttd, J=17.4, 6.2, 4.5 Hz, 2H).

### 2-(3,3-Difluoropropoxy)pyrimidin-5-amine

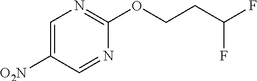 [SMILES: O=[N+]([O-])c1cnc(OCCC(F)F)nc1]

Prepared from 2-(3,3-difluoropropoxy)-5-nitropyrimidine according to General Procedure M to afford the desired product 2-(3,3-difluoropropoxy)pyrimidin-5-amine (84 mg, 0.439 mmol, 84% yield) as a yellow solid. ¹H NMR (400 MHz, DMSO-D6) δ 7.95 (s, 2H), 6.21 (tt, J=56.4, 4.6 Hz, 1H), 5.02 (s, 2H), 4.27 (t, J=6.4 Hz, 2H), 2.38-2.06 (m, 2H). UPLC-MS (Basic 4 min): rt=0.81 min, m/z (ES+) 190.1 [M+H]⁺.

### 5-Nitro-2-(3,3,3-trifluoropropoxy)pyrimidine

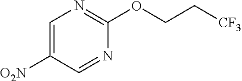 [SMILES: O=[N+]([O-])c1cnc(OCCC(F)(F)F)nc1]

Prepared from 2-chloro-5-nitropyrimidine and 3,3,3-trifluoropropan-1-ol according to General Procedure L to afford the desired product 5-nitro-2-(3,3,3-trifluoropropoxy)pyrimidine (104 mg, 0.438 mmol, 23% yield) as a brown oil. ¹H NMR (400 MHz, DMSO-D6) δ 9.02 (s, 1H), 4.76-4.60 (m, 2H), 2.90 (qt, J=11.4, 5.9 Hz, 2H).

### 2-(3,3,3-Trifluoropropoxy)pyrimidin-5-amine

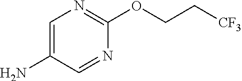 [SMILES: Nc1cnc(OCCC(F)(F)F)nc1]

Prepared from 5-nitro-2-(3,3,3-trifluoropropoxy)pyrimidine according to General Procedure M to afford the desired product 2-(3,3,3-trifluoropropoxy)pyrimidin-5-amine (64 mg, 0.296 mmol, 70% yield) as a yellow solid. ¹H NMR (400 MHz, DMSO-D6) δ 7.96 (s, 2H), 5.04 (s, 2H), 4.35 (t, J=6.1 Hz, 2H), 2.86-2.65 (m, 2H). UPLC-MS (Basic 4 min): rt=0.99 min, m/z (ES+) 208.1 [M+H]⁺.

### 2-(3-(Difluoromethyl)azetidin-1-yl)-5-nitropyrimidine

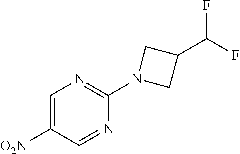 [SMILES: O=[N+]([O-])c1cnc(N2CC(C(F)F)C2)nc1]

2-Chloro-5-nitropyrimidine (200 mg, 1.25 mmol), 3-(difluoromethyl)azetidine hydrochloride (216 mg, 1.50 mmol) and N,N-diisopropylethylamine (0.66 mL, 3.79 mmol) were dissolved in NMP (5 mL) and the resultant solution was stirred at rt for 30 minutes. Water (10 mL) was added to the reaction mixture and the obtained precipitate was collected by filtration, washed with additional H₂O (5 mL) and dried under reduced pressure to afford the desired product 2-(3-(difluoromethyl)azetidin-1-yl)-5-nitropyrimidine (264 mg, 1.14 mmol, 91% yield) as an off-white solid. ¹H NMR (400 MHz, DMSO-D6) δ 9.08 (s, 1H), 6.38 (td, J=56.2, 4.2 Hz, 1H), 4.37-4.32 (m, 2H), 4.17-4.13 (m, 4H), 3.31-3.27 (m, 1H).

### 2-(3-(Difluoromethyl)azetidin-1-yl)pyrimidin-5-amine

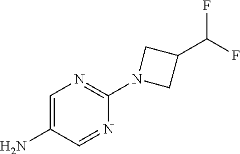 [SMILES: Nc1cnc(N2CC(C(F)F)C2)nc1]

Prepared from 2-(3-(difluoromethyl)azetidin-1-yl)-5-nitropyrimidine according to General Procedure M to afford the desired product 2-(3-(difluoromethyl)azetidin-1-yl)pyrimidin-5-amine (210 mg, 1.04 mmol, 91% yield) as a brown solid. ¹H NMR (400 MHz, DMSO-D6) δ 7.84 (s, 2H), 6.29 (td, J=56.6, 4.6 Hz, 1H), 4.62 (s, 2H), 3.92 (t, J=8.5 Hz, 2H), 3.76 (dd, J=8.6, 5.6 Hz, 3H), 3.18-2.99 (m, 1H). UPLC-MS (Basic 2 min): rt=0.66 min, m/z (ES+) 201.3 [M+H]⁺.

### 5-Bromo-2-chloro-3-(oxiran-2-ylmethoxy)pyridine

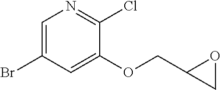 [SMILES: Clc1ncc(Br)cc1OCC1CO1]

To a suspension of sodium hydride (60% disp. in mineral oil, 460 mg, 11.5 mmol) in DMF (5 mL) was added a solution of 5-bromo-2-chloro-pyridin-3-ol (2.00 g, 9.60 mmol) in DMF (2 mL). This was stirred for 0.5 h at rt before a solution of epibromohydrin (1.31 g, 9.60 mmol) in DMF (2 mL) was added dropwise. The reaction was heated at 60° C. for 22 h. The reaction mixture was directly purified by column chromatography over C18 eluting with a gradient of MeCN(0.1% NH₃) (5% to 95%; v/v) in water (0.1% NH₃) to afford the desired product 5-bromo-2-chloro-3-(oxiran-2-ylmethoxy)pyridine (1.21 g, 4.59 mmol, 48% yield) as a brown solid. ¹H NMR (400 MHz, DMSO-D6) δ 8.15 (d, J=2.0 Hz, 1H), 7.90 (d, J=2.0 Hz, 1H), 4.58 (dd, J=11.7, 2.4 Hz, 1H), 4.07 (dd, J=11.7, 6.5 Hz, 1H), 3.38 (ddt, J=6.6, 4.2, 2.5 Hz, 1H), 2.87 (dd, J=5.1, 4.3 Hz, 1H), 2.73 (dd, J=5.1, 2.6 Hz, 1H). UPLC-MS (Basic 2 min): rt=1.49 min, m/z=264.1/266. 1/268.1 [M+H]⁺.

### 1-((5-Bromo-2-chloropyridin-3-yl)oxy)-3-methoxypropan-2-ol

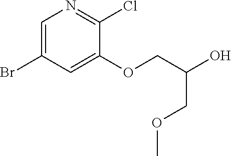

5-Bromo-2-chloro-3-(oxiran-2-ylmethoxy)pyridine (550) mg. 2.08 mmol) and sodium methoxide (562 mg, 10.4 mmol) dissolved in methanol (20 mL) were heated at 40° C. for 22 h. This was concentrated under reduced pressure to afford the crude product. This was purified by column chromatography over C18 eluting with a gradient of MeCN(0.1% NH₃) (5% to 95%; v/v) in water (0.1% NH₃) to afford the desired product 1-((5-bromo-2-chloropyridin-3-yl)oxy)-3-methoxypropan-2-ol (470) mg. 1.33 mmol, 64% yield) as a beige solid. ¹H NMR (400 MHz, DMSO-D6) δ 8.11 (d, J=2.0 Hz, 1H), 7.90 (d, J=2.1 Hz, 1H), 5.18 (d, J=3.2 Hz, 1H), 4.16 (dd, J=10.4, 4.3 Hz, 1H), 4.10 (dd, J=10.4, 5.6 Hz, 1H), 4.00-3.91 (m, 1H), 3.48-3.36 (m, 2H), 3.29 (s, 3H). UPLC-MS (Basic 2 min): rt=2.11 min, m/z=296.0, 298.0 [M+H]⁺.

### 7-Bromo-3-(methoxymethyl)-2,3-dihydro-[1,4]dioxino[2,3-b]pyridine and 7-Bromo-2-(methoxymethyl)-2,3-dihydro-[1,4]dioxino[2,3-b]pyridine

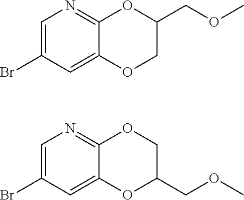

Sodium tert-butoxide (379 mg, 3.95 mmol) was added to a solution of 1-((5-bromo-2-chloropyridin-3-yl)oxy)-3-methoxypropan-2-ol (470) mg. 1.33 mmol) in 1-butanol (10.5 mL) and heated at 80° C. for 18 h. This was concentrated under reduced pressure to afford the crude product. This was purified by column chromatography over C18 eluting with a gradient of MeCN(0.1% NH₃) (5% to 95%; v/v) in water (0.1% NH₃) to afford the desired products 7-bromo-3-(methoxymethyl)-2,3-dihydro-[1,4]dioxino[2,3-b]pyridine and 7-bromo-2-(methoxymethyl)-2,3-dihydro-[1,4]dioxino[2,3-b]pyridine (156 mg, 0.600 mmol, 38% yield) as a white solid. ¹H NMR (400 MHz, DMSO-D6) δ 7.78 (d, J=2.1 Hz, 1H), 7.51 (d, J=2.1 Hz, 1H), 5.10 (d, J=5.0 Hz, 1H), 4.26 (t. J=6.6 Hz, 2H), 4.01-3.87 (m, 2H), 3.27 (s, 3H).

### N-(3-(Methoxymethyl)-2,3-dihydro-[1,4]dioxino[2,3-b]pyridin-7-yl)-1,1-diphenylmethanimine and N-(2-(Methoxymethyl)-2,3-dihydro-[1,4]dioxino[2,3-b]pyridin-7-yl)-1,1-diphenylmethanimine

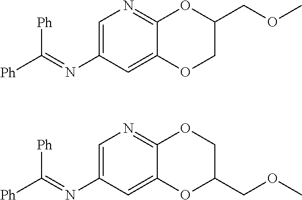 [SMILES: COCC1COc2cc(N=C(c3ccccc3)c3ccccc3)cnc2O1 ; COCC1COc2ncc(N=C(c3ccccc3)c3ccccc3)cc2O1]

A mixture of 7-bromo-3-(methoxymethyl)-2,3-dihydro-[1,4]dioxino[2,3-b]pyridine and 7-bromo-2-(methoxymethyl)-2,3-dihydro-[1,4]dioxino[2,3-b]pyridine (130 mg, 0.500) mmol), benzophenoneimine (109 mg, 0.600 mmol) and sodium tert-butoxide (67 mg, 0.700 mmol) in dimethoxyethane (2 mL) was sparged with N₂ for 10 min, and Josiphos SL-J009-1-G3-palladacycle (4.7 mg, 0.00500 mmol) was added. The reaction mixture was bubbled with N₂ for further 5 min, and then stirred at 100° C. for 70 h. This was cooled to room temperature and concentrated under reduced pressure to afford the crude product. This was purified by column chromatography over C18 eluting with a gradient of MeCN(0.1% NH₃) (5% to 95%; v/v) in water (0.1% NH₃) to afford the desired products N-(3-(methoxymethyl)-2,3-dihydro-[1,4]dioxino[2,3-b]pyridin-7-yl)-1,1-diphenylmethanimine and N-(2-(methoxymethyl)-2,3-dihydro-[1,4]dioxino[2,3-b]pyridin-7-yl)-1,1-diphenylmethanimine (91 mg, 0.247 mmol, 50% yield). ¹H NMR (400 MHz, DMSO-D6) δ 7.67-7.62 (m, 2H), 7.56-7.50 (m, 1H), 7.49-7.43 (m, 2H), 7.41-7.34 (m, 3H), 7.22-7.16 (m, 2H), 7.13 (dd, J=4.5, 2.3 Hz, 1H), 6.75 (dd, J=5.3, 2.3 Hz, 1H), 4.25 (dd, J=11.6, 2.4 Hz, 2H), 4.06 (dd, J=11.7, 7.5 Hz, 1H), 3.91 (dd, J=11.6, 7.7 Hz, 1H), 3.58-3.49 (m, 2H), 3.30 (s, 3H). UPLC-MS (Basic 4 min): rt=3.41 min, m/z=361.2 [M+H]⁺, rt=3.46 min, m/z=361.2 [M+H]⁺.

### 3-(Methoxymethyl)-2,3-dihydro-[1,4]dioxino[2,3-b]pyridin-7-amine and 2-(Methoxymethyl)-2,3-dihydro-[1,4]dioxino[2,3-b]pyridin-7-amine

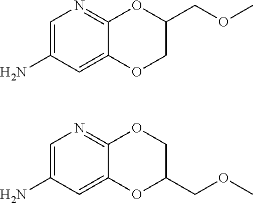

N-(3-(Methoxymethyl)-2,3-dihydro-[1,4]dioxino[2,3-b]pyridin-7-yl)-1, 1-diphenylmethanimine and N-(2-(methoxymethyl)-2,3-dihydro-[1,4]dioxino[2,3-b]pyridin-7-yl)-1, 1-diphenylmethanimine (68 mg, 0.189 mmol) and Palladium on activated Carbon (10% loading, paste, 71 mg, 0.0332 mmol) were suspended in IPA (4 mL) and stirred under an atmosphere of H₂ for 18 h. This was filtered through celite, washing with IPA, and concentrated under reduced pressure to afford the desired products 3-(methoxymethyl)-2,3-dihydro-[1,4]dioxino[2,3-b]pyridin-7-amine and 2-(methoxymethyl)-2,3-dihydro-[1,4]dioxino[2,3-b]pyridin-7-amine (36 mg, 0.183 mmol, 97% yield) as a yellow solid. ¹H NMR (400 MHz, DMSO-D6) δ 7.12 (dd, J=5.3, 2.5 Hz, 1H), 6.55 (dd, J=2.5, 0.7 Hz, 1H), 4.81 (s, 2H), 4.35-4.17 (m, 2H), 3.90 (dd, J=11.4, 7.6 Hz, 1H), 3.62-3.49 (m, 2H), 3.29 (s, 3H).

### (R)—N-(Tetrahydro-2H-pyran-3-yl)-5,6-dihydrobenzo[f]imidazo[1,5-d][1,4]oxazepine-10-carboxamide 1A

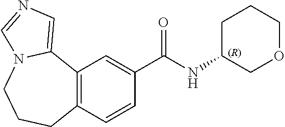

Prepared from lithium 5,6-dihydrobenzo[f]imidazo[1,5-d][1,4]oxazepine-10-carboxylate 4 and (R)-tetrahydro-2H-pyran-3-amine hydrochloride according to General Procedure E to afford the desired product (R)—N-(tetrahydro-2H-pyran-3-yl)-5,6-dihydrobenzo[f]imidazo[1,5-d][1,4]oxazepine-10-carboxamide 1A (64 mg, 0.203 mmol, 39% yield) as a white solid. ¹H NMR (400 MHz, DMSO-D6) δ 8.25 (d, J=2.2 Hz, 1H), 8.20 (d, J=7.7 Hz, 1H), 7.77 (d, J=1.1 Hz, 1H), 7.66 (dd, J=8.6, 2.2 Hz, 1H), 7.61 (d, J=1.1 Hz, 1H), 7.04 (d, J=8.5 Hz, 1H), 4.52-4.39 (m, 4H), 3.97-3.86 (m, 1H), 3.85-3.74 (m, 2H), 3.42-3.24 (m, 1H), 3.20-3.12 (m, 1H), 1.98-1.86 (m, 1H), 1.77-1.52 (m, 3H). UPLC-MS (Basic 4 min): rt=1.09 min, m/z (ES+) 314.1 [M+H]⁺.

### (R)—N-(1,1-Dioxidotetrahydrothiophen-3-yl)-5,6-dihydrobenzo[f]imidazo[1,5-d][1,4]oxazepine-10-carboxamide 2A

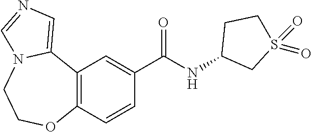 [SMILES: O=C(N[C@@H]1CCS(=O)(=O)C1)c1ccc2c(c1)-c1cncn1CCO2]

Prepared from lithium 5,6-dihydrobenzo[f]imidazo[1,5-d][1,4]oxazepine-10-carboxylate 4 and (3R)-1,1-dioxothiolan-3-amine hydrochloride according to General Procedure E to afford the desired product (R)—N-(1,1-dioxidotetrahydrothiophen-3-yl)-5,6-dihydrobenzo[f]imidazo[1,5-d][1,4]oxazepine-10-carboxamide 2A (16 mg, 0.0459 mmol, 36% yield) as an off-white solid. ¹H NMR (400 MHz, DMSO-D6) δ 8.71 (d, J=7.1 Hz, 1H), 8.27 (d, J=2.3 Hz, 1H), 7.79 (s, 1H), 7.71-7.58 (m, 2H), 7.07 (d, J=8.5 Hz, 1H), 4.78-4.63 (m, 1H), 4.58-4.38 (m, 4H), 3.52 (dd, J=13.4, 8.1 Hz, 1H), 3.44-3.30 (m, 1H), 3.28-3.14 (m, 1H), 3.08 (dd, J=13.4, 8.1 Hz, 1H), 2.59-2.39 (m, 1H), 2.30-2.16 (m, 1H). UPLC-MS (Basic 4 min): rt=0.94 min, m/z (ES+) 348.3 [M+H]⁺.

### N-((1r,4r)-4-(2-Methoxyethoxy)cyclohexyl)-5,6-dihydrobenzo[f]imidazo[1,5-d][1,4]oxazepine-10-carboxamide 3A

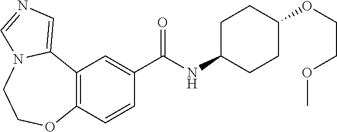 [SMILES: COCCO[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and trans-4-(2-methoxyethoxy)cyclohexanamine according to General Procedure E to afford the desired product N-((1r,4r)-4-(2-methoxyethoxy)cyclohexyl)-5,6-dihydrobenzo[f]imidazo[1,5-d][1,4]oxazepine-10-carboxamide 3A (12 mg, 0.031 mmol, 24% yield) as an off-white solid. ¹H NMR (400 MHz, DMSO-D6) δ 8.27-8.13 (m, 2H), 7.77 (s, 1H), 7.70-7.55 (m, 2H), 7.03 (d, J=8.6 Hz, 1H), 4.55-4.34 (m, 4H), 3.83-3.68 (m, 1H), 3.61-3.48 (m, 2H), 3.47-3.38 (m, 2H), 3.30-3.19 (m, 4H), 2.10-1.95 (m, 2H), 1.92-1.80 (m, 2H), 1.46-1.31 (m, 2H), 1.29-1.15 (m, 2H). UPLC-MS (Basic 4 min): rt=1.22 min, m z (ES+) 386.5 [M+H]⁺.

### N-(1-(Methylsulfonyl) piperidin-4-yl)-5,6-dihydrobenzo[f]imidazo[1,5-d][1,4]oxazepine-10-carboxamide 4A

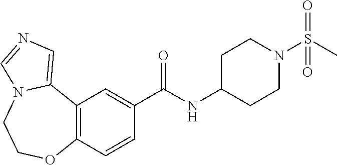 [SMILES: CS(=O)(=O)N1CCC(NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and 1-(methylsulfonyl)-4-piperidinamine according to General Procedure E to afford the desired product N-(1-(methylsulfonyl) piperidin-4-yl)-5,6-dihydrobenzo[f]imidazo[1,5-d][1,4]oxazepine-10-carboxamide 4A (21 mg, 0.0543 mmol, 43% yield) as an off-white solid. 1H NMR (400 MHz, DMSO-D6) δ 8.33 (d, J=7.7 Hz, 1H), 8.28-8.22 (m, 1H), 7.78 (s, 1H), 7.70-7.58 (m, 2H), 7.05 (d, J=8.5 Hz, 1H), 4.54-4.38 (m, 4H), 3.99-3.85 (m, 1H), 3.64-3.53 (m, 2H), 2.93-2.80 (m, 5H), 1.99-1.87 (m, 2H), 1.68-1.54 (m, 2H) UPLC-MS (Basic 4 min): rt=1.07 min, m/z (ES+) 391.4 [M+H]⁺.

### N-(4,4-Difluorocyclohexyl)-5,6-dihydrobenzo[f]imidazo[1,5-d][1,4]oxazepine-10-carboxamide 5A

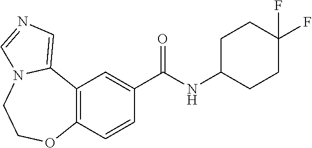 [SMILES: O=C(NC1CCC(F)(F)CC1)c1ccc2c(c1)-c1cncn1CCO2]

Prepared from lithium 5,6-dihydrobenzo[f]imidazo[1,5-d][1,4]oxazepine-10-carboxylate 4 and 4,4-difluorocyclohexanamine according to General Procedure E to afford the desired product N-(4,4-difluorocyclohexyl)-5,6-dihydrobenzo[f]imidazo[1,5-d][1,4]oxazepine-10-carboxamide 5A (12 mg, 0.0342 mmol, 27% yield) as a white solid. ¹H NMR (400 MHz, DMSO-D6) δ 8.26-8.18 (m, 2H), 7.74 (d, J=1.1 Hz, 1H), 7.63 (dd, J=8.6, 2.2 Hz, 1H), 7.57 (d, J=1.1 Hz, 1H), 7.01 (d, J=8.6 Hz, 1H), 4.47-4.36 (m, 4H), 3.98-3.96 (m, 1H), 2.07-1.81 (m, 6H), 1.69-1.59 (m, 2H). ¹⁹F NMR (376 MHz, DMSO-D6) δ-91.9 (d, J 233 Hz), −98.6 (d, J 233 Hz). UPLC-MS (Basic 4 min): rt=1.34 min, m/z (ES+) 348.4 [M+H]⁺.

### (S)—N-(2,3-Dihydro-1H-inden-1-yl)-5,6-dihydrobenzo[f]imidazo[1,5-d][1,4]oxazepine-10-carboxamide 6A

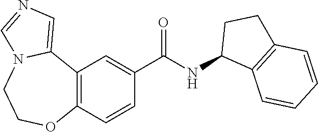 [SMILES: O=C(N[C@H]1CCc2ccccc21)c1ccc2c(c1)-c1cncn1CCO2]

Prepared from lithium 5,6-dihydrobenzo[f]imidazo[1,5-d][1,4]oxazepine-10-carboxylate 4 and (1S)-indan-1-amine according to General Procedure E to afford the desired product(S)—N-(2,3-dihydro-1H-inden-1-yl)-5,6-dihydrobenzo[f]imidazo[1,5-d][1,4]oxazepine-10-carboxamide 6A (20 mg, 0.0573 mmol, 45% yield) as a white solid. ¹H NMR (400 MHz, DMSO-D6) δ 8.76 (d, J=8.4 Hz, 1H), 8.31 (d, J=2.2 Hz, 1H), 7.73-7.70 (m, 2H), 7.57 (d, J=1.1 Hz, 1H), 7.28-7.14 (m, 4H), 7.02 (d, J=8.6 Hz, 1H), 5.55 (q, J=8.2 Hz, 1H), 4.48-4.36 (m, 4H), 2.96-2.93 (m, 1H), 2.83-2.81 (m, 1H), 2.47-2.36 (m, 1H), 2.06-1.88 (m, 1H). UPLC-MS (Basic 4 min): rt=1.49 min, m/z (ES+) 346.4 [M+H]⁺.

### N-(Benzo[d]thiazol-5-yl)-5,6-dihydrobenzo[f]imidazo[1,5-d][1,4]oxazepine-10-carboxamide 7A

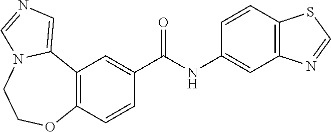 [SMILES: O=C(Nc1ccc2scnc2c1)c1ccc2c(c1)-c1cncn1CCO2]

Prepared from lithium 5,6-dihydrobenzo[f]imidazo[1,5-d][1,4]oxazepine-10-carboxylate 4 and 1,3-benzothiazol-5-amine according to General Procedure E to afford the desired product N-(benzo[d]thiazol-5-yl)-5,6-dihydrobenzo[f]imidazo[1,5-d][1,4]oxazepine-10-carboxamide 7A (55 mg, 0.152 mmol, 36% yield) as a brown solid. ¹H NMR (400 MHz, DMSO-D6) δ 10.41 (s, 1H), 9.36 (s, 1H), 8.58 (d, J=2.0 Hz, 1H), 8.39 (d, J=2.3 Hz, 1H), 8.10 (d, J=8.7 Hz, 1H), 7.83-7.77 (m, 2H), 7.77 (d, J=1.4 Hz, 1H), 7.66 (d, J=1.1 Hz, 1H), 7.56-7.54 (m, 1H), 7.11 (d, J=8.6 Hz, 1H), 4.51-4.41 (m, 4H). UPLC-MS (Basic 4 min): rt=1.32 min, m/z (ES+) 363.1 [M+H]⁺.

### N-(2,3-Dihydrobenzo[b][1,4]dioxin-6-yl)-5,6-dihydrobenzo[f]imidazo[1,5-d][1,4]oxazepine-10-carboxamide 8A

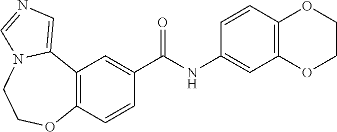 [SMILES: O=C(Nc1ccc2c(c1)OCCO2)c1ccc2c(c1)-c1cncn1CCO2]

Prepared from lithium 5,6-dihydrobenzo[f]imidazo[1,5-d][1,4]oxazepine-10-carboxylate 4 and 1,4-benzodioxan-6-amine according to General Procedure E to afford the desired product N-(2,3-dihydrobenzo[b][1,4]dioxin-6-yl)-5,6-dihydrobenzo[f]imidazo[1,5-d][1,4]oxazepine-10-carboxamide 8A (46 mg, 0.122 mmol, 29% yield) as an off-white solid. 1H NMR (400 MHz, DMSO-D6) δ 10.04 (s, 1H), 8.35 (d, J=2.3 Hz, 1H), 7.79 (d, J=1.1 Hz, 1H), 7.74 (dd, J=8.5, 2.2 Hz, 1H), 7.66 (d, J=1.1 Hz, 1H), 7.38 (d, J=2.5 Hz, 1H), 7.19 (dd, J=8.7, 2.5 Hz, 1H), 7.10 (d, J=8.6 Hz, 1H), 6.83 (d, J=8.7 Hz, 1H), 4.53-4.43 (m, 4H), 4.28-4.20 (m, 4H). UPLC-MS (Basic 4 min): rt=1.36 min, m/z (ES+) 364.3 [M+H]⁺.

### N-(2,3-Dihydrobenzofuran-5-yl)-5,6-dihydrobenzo[f]imidazo[1,5-d][1,4]oxazepine-10-carboxamide 9A

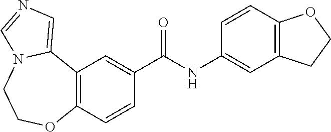 [SMILES: O=C(Nc1ccc2c(c1)CCO2)c1ccc2c(c1)-c1cncn1CCO2]

Prepared from lithium 5,6-dihydrobenzo[f]imidazo[1,5-d][1,4]oxazepine-10-carboxylate 4 and 5-amino-2,3-dihydrobenzo[b]furan according to General Procedure E to afford the desired product N-(2,3-dihydrobenzofuran-5-yl)-5,6-dihydrobenzo[f]imidazo[1,5-d][1,4]oxazepine-10-carboxamide 9A (37 mg, 0.102 mmol, 24% yield) as an off-white solid. 1H NMR (400 MHz, DMSO-D6) δ 10.04 (s, 1H), 8.36 (d, J=2.2 Hz, 1H), 7.82-7.71 (m, 2H), 7.69-7.61 (m, 2H), 7.39 (dd, J=8.5, 2.3 Hz, 1H), 7.10 (d, J=8.6 Hz, 1H), 6.74 (d, J=8.5 Hz, 1H), 4.56-4.43 (m, 6H), 3.19 (t, J=8.7 Hz, 2H). UPLC-MS (Basic 4 min): rt=1.36 min, m/z (ES+) 348.3 [M+H]⁺.

### N-(6-(Trifluoromethyl)pyridin-3-yl)-5,6-dihydrobenzo[f]imidazo[1,5-d][1,4]oxazepine-10-carboxamide 10A

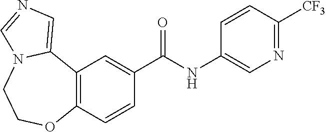 [SMILES: O=C(Nc1ccc(C(F)(F)F)nc1)c1ccc2c(c1)-c1cncn1CCO2]

Prepared from lithium 5,6-dihydrobenzo[f]imidazo[1,5-d][1,4]oxazepine-10-carboxylate 4 and 5-amino-2-(trifluoromethyl)pyridine according to General Procedure E to afford the desired product N-(6-(trifluoromethyl)pyridin-3-yl)-5,6-dihydrobenzo[f]imidazo[1,5-d][1,4]oxazepine-10-carboxamide 10A (8 mg, 0.0212 mmol, 5% yield) as a white solid. 1H NMR (400 MHz, DMSO-D6) δ 10.76 (s, 1H), 9.11 (d, J=2.4 Hz, 1H), 8.49 (dd, J=8.5, 2.4 Hz, 1H), 8.44 (d, J=2.2 Hz, 1H), 7.93 (d, J=8.7 Hz, 1H), 7.85-7.77 (m, 2H), 7.68 (d, J=1.2 Hz, 1H), 7.16 (d, J=8.6 Hz, 1H), 4.55-4.45 (m, 4H). ¹⁹F NMR (376 MHz, DMSO-D6) δ-65.5 (s). UPLC-MS (Basic 4 min): rt=1.52 min, m/z (ES+) 375.1 [M+H]⁺.

### N-((1r,4r)-4-(2-Methoxyethoxy)cyclohexyl)-4-oxo-4,5-dihydroimidazo[1,5-a]quinoxaline-8-carboxamide 11A

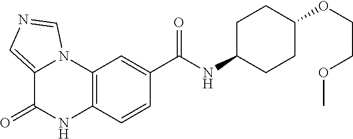 [SMILES: COCCO[C@H]1CC[C@H](NC(=O)c2ccc3[nH]c(=O)c4cncn4c3c2)CC1]

Prepared from lithium 4-oxo-4,5-dihydroimidazo[1,5-a]quinoxaline-8-carboxylate 9 and trans-4-(2-methoxyethoxy)cyclohexanamine according to General Procedure E to afford the desired product N-((1r,4r)-4-(2-methoxyethoxy)cyclohexyl)-4-oxo-4,5-dihydroimidazo[1,5-a]quinoxaline-8-carboxamide 11A (10 mg, 0.026 mmol, 12% yield) as a grey solid. ¹H NMR (400 MHz, DMSO-D6) δ 11.61 (s, 1H), 9.06 (s, 1H), 8.59 (d, J=1.9 Hz, 1H), 8.19 (d, J=7.7 Hz, 1H), 7.89 (s, 1H), 7.83 (dd, J=8.5, 1.8 Hz, 1H), 7.33 (d, J=8.5 Hz, 1H), 3.84-3.72 (m, 1H), 3.54 (dd, J=5.9, 3.8 Hz, 2H), 3.42 (dd, J=5.9, 3.8 Hz, 2H), 3.25 (s, 4H), 2.03 (d, J=12.1 Hz, 2H), 1.91 (d, J=12.4 Hz, 2H), 1.37 (dd, J=13.7, 10.9 Hz, 2H), 1.25 (q, J=11.3 Hz, 2H). UPLC-MS (Basic 6 min): rt=1.05 min, m/z (ES+) 385.2 [M+H]⁺.

### (R)—N-(1,1-Dioxidotetrahydrothiophen-3-yl)-4-oxo-4,5-dihydroimidazo[1,5-a]quinoxaline-8-carboxamide 12A

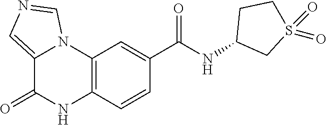 [SMILES: O=C(N[C@@H]1CCS(=O)(=O)C1)c1ccc2[nH]c(=O)c3cncn3c2c1]

Prepared from lithium 4-oxo-4,5-dihydroimidazo[1,5-a]quinoxaline-8-carboxylate 9 and (3R)-1,1-dioxothiolan-3-amine hydrochloride according to General Procedure E to afford the desired product (R)—N-(1,1-dioxidotetrahydrothiophen-3-yl)-4-oxo-4,5-dihydroimidazo[1,5-a]quinoxaline-8-carboxamide 12A (18 mg, 0.053 mmol, 25% yield) as a white solid. ¹H NMR (400 MHz, DMSO-D6) δ 9.07 (d, J=0.8 Hz, 1H), 8.78 (d, J=7.1 Hz, 1H), 8.61 (d, J=1.8 Hz, 1H), 7.90-7.81 (m, 2H), 7.35 (d, J=8.5 Hz, 1H), 4.80-4.66 (m, 1H), 3.55 (dd, J=13.5, 8.1 Hz, 1H), 3.46-3.38 (m, 1H), 3.23 (ddd, J=13.3, 10.0, 7.4 Hz, 1H), 3.08 (ddd, J=13.5, 8.0, 1.1 Hz, 1H), 2.50-2.41 (m, 1H), 2.24 (dtd, J=13.2, 9.7, 7.9 Hz, 1H). UPLC-MS (Basic 4 min): rt=0.77 min, m/z (ES+) 347.0 [M+H]⁺.

### (R)-4-Oxo-N-(tetrahydro-2H-pyran-3-yl)-4,5-dihydroimidazo[1,5-a]quinoxaline-8-carboxamide 13A

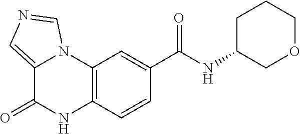 [SMILES: O=C(N[C@@H]1CCCOC1)c1ccc2[nH]c(=O)c3cncn3c2c1]

Prepared from lithium 4-oxo-4,5-dihydroimidazo[1,5-a]quinoxaline-8-carboxylate 9 and (R)-tetrahydro-2H-pyran-3-amine hydrochloride according to General Procedure E to afford the desired product (R)-4-oxo-N-(tetrahydro-2H-pyran-3-yl)-4,5-dihydroimidazo[1,5-a]quinoxaline-8-carboxamide 13A (18 mg, 0.058 mmol, 27% yield) as a white solid. ¹H NMR (400 MHz, DMSO-D6) δ 11.58 (s, 1H), 9.08 (d, J=0.8 Hz, 1H), 8.63 (d, J=1.8 Hz, 1H), 8.23 (d, J=7.7 Hz, 1H), 7.89 (d, J=0.8 Hz, 1H), 7.85 (dd, J=8.5, 1.8 Hz, 1H), 7.33 (d, J=8.5 Hz, 1H), 3.98-3.90 (m, 1H), 3.88-3.81 (m, 1H), 3.80-3.75 (m, 1H), 3.33-3.30 (m, 1H), 3.20 (dd, J=10.7, 9.2 Hz, 1H), 2.03-1.88 (m, 1H), 1.78-1.52 (m, 3H). UPLC-MS (Basic 4 min): rt=0.89 min, m/z (ES+) 313.1 [M+H]⁺.

### (R)—N-(Tetrahydro-2H-pyran-3-yl)-4,5-dihydrobenzo[b]imidazo[1,2-d][1,4]oxazepine-9-carboxamide 14A

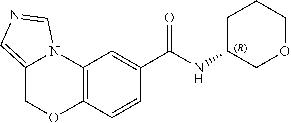

Prepared from lithium 4,5-dihydrobenzo[b]imidazo[1,2-d][1,4]oxazepine-9-carboxylate 12 and (R)-tetrahydro-2H-pyran-3-amine hydrochloride according to General Procedure E to afford the desired product (R)—N-(tetrahydro-2H-pyran-3-yl)-4,5-dihydrobenzo[b]imidazo[1,2-d][1,4]oxazepine-9-carboxamide 14A (21 mg, 0.068 mmol, 39% yield) as a white solid. ¹H NMR (400 MHz, DMSO-D6) δ 8.29 (d, J=7.7 Hz, 1H), 7.99 (d, J=2.1 Hz, 1H), 7.82 (dd, J=8.4, 2.2 Hz, 1H), 7.66 (d, J=1.4 Hz, 1H), 7.33 (d, J=8.4 Hz, 1H), 7.03 (d, J=1.5 Hz, 1H), 4.56 (t, J=6.3 Hz, 2H), 3.97-3.86 (m, 1H), 3.85-3.72 (m, 2H), 3.40-3.25 (m, 1H), 3.21-3.12 (m, 1H), 3.05 (t, J=6.3 Hz, 2H), 1.98-1.86 (m, 1H), 1.77-1.50 (m, 3H). UPLC-MS (Basic 4 min): rt=1.07 min, m/z (ES+) 314.3 [M+H]⁺.

### (R)—N-(Tetrahydro-2H-pyran-3-yl)-4,5-dihydrobenzo[b]imidazo[1,5-d][1,4]oxazepine-9-carboxamide 15A

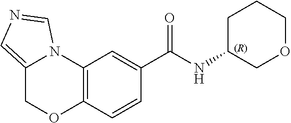

A suspension of methyl 4-(2-(1H-imidazol-5-yl)ethoxy)-3-bromobenzoate 14 (170 mg, 0.507 mmol), G3-Pd-Me4tBuXPhos (22 mg, 0.0254 mmol) and potassium phosphate tribasic (323 mg, 1.52 mmol) in 1,4-dioxane (3 mL) was heated to 100° C. for 3 h. Further G3-Pd-Me4tBuXPhos (22 mg, 0.0254 mmol) was added and heating continued for a further 3 days. Further G3-Pd-Me4tBuXPhos (22 mg, 0.0254 mmol) and potassium phosphate tribasic (323 mg, 1.52 mmol) were added and heating continued for a further 24 h. Reaction mixture was loaded onto an SCX (1 g) cartridge flushing first with methanol, then 2M NH₃/MeOH followed by MeCN: H₂O (1:1). The aqueous fraction was concentrated under reduced pressure to give a yellow oil. The crude material was purified by column chromatography over C18 eluting with a gradient of MeCN(0.1% NH₃) (0% to 50%; v/v) in water (0.1% NH₃) to afford impure intermediate acid (100 mg) as a yellow gum. This was suspended in MeCN(2 mL) and chloro-N,N,N′,N′-tetramethylformamidinium hexafluorophosphate (161 mg, 0.573 mmol), (R)-tetrahydro-2H-pyran-3-amine hydrochloride (50 mg, 0.365 mmol) and 1-methylimidazole (0.093 mL, 1.17 mmol) were added and the reaction mixture was allowed to stir for 18 h. This was concentrated under reduced pressure and purified by column chromatography over C18 eluting with a gradient of MeCN(0.1% NH₃) (0% to 60%; v/v) in water (0.1% NH₃) followed by prep HPLC purification to afford the desired product (R)—N-(tetrahydro-2H-pyran-3-yl)-4,5-dihydrobenzo[b]imidazo[1,5-d][1,4]oxazepine-9-carboxamide 15A (7.4 mg, 0.0236 mmol, 9% yield) as a white solid. ¹H NMR (400 MHz, DMSO-D6) δ 8.26 (d, J=7.7 Hz, 1H), 8.10 (d, J=1.0 Hz, 1H), 8.06 (d, J=2.2 Hz, 1H), 7.85 (dd, J=8.4, 2.2 Hz, 1H), 7.33 (d, J=8.4 Hz, 1H), 6.95 (d, J=0.9 Hz, 1H), 4.44 (t, J=6.3 Hz, 2H), 3.93-391 (m, 1H), 3.87-3.72 (m, 2H), 3.29 (dd, J=10.9, 2.8 Hz, 1H), 3.17 (dd, J=10.7, 9.3 Hz, 1H), 2.96 (t, J=6.3 Hz, 2H), 1.92 (dd, J=4.6, 4.6 Hz, 1H), 1.73-1.71 (m, 1H), 1.69-1.51 (m, 2H). UPLC-MS (Basic 4 min): rt=1.11 min, m/z (ES+) 314.1 [M+H]⁺.

### (R)-5-Methyl-4-oxo-N-(tetrahydro-2H-pyran-3-yl)-4,5-dihydroimidazo[1,5-a]quinoxaline-8-carboxamide 16A

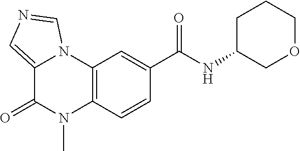 [SMILES: Cn1c(=O)c2cncn2c2cc(C(=O)N[C@@H]3CCCOC3)ccc21]

Prepared from lithium 5-methyl-4-oxo-4,5-dihydroimidazo[1,5-a]quinoxaline-8-carboxylate 16 and (R)-tetrahydro-2H-pyran-3-amine hydrochloride according to General Procedure E to afford the desired product (R)-5-methyl-4-oxo-N-(tetrahydro-2H-pyran-3-yl)-4,5-dihydroimidazo[1,5-a]quinoxaline-8-carboxamide 16A (50 mg, 0.152 mmol, 34% yield) as a beige solid. ¹H NMR (400 MHz, DMSO-D6) δ 9.11 (s, 1H), 8.67 (s, 1H), 8.30 (d, J=7.7 Hz, 1H), 7.99-7.89 (m, 2H), 7.61 (d, J=8.8 Hz, 1H), 3.96 (m, 1H), 3.89-3.82 (m, 1H), 3.78 (d, J=11.4 Hz, 1H), 3.58 (s, 3H), 3.22 (t, J=10.1 Hz, 2H), 1.98 (s, 1H), 1.74 (d, J=11.5 Hz, 1H), 1.69-1.59 (m, 2H). UPLC-MS (Basic 4 min): rt=1.01 min, m/z (ES+) 327.3 [M+H]⁺.

### N-((1r,4r)-4-(Methylcarbamoyl)cyclohexyl)-5,6-dihydrobenzo[f]imidazo[1,5-d][1,4]oxazepine-10-carboxamide 17A

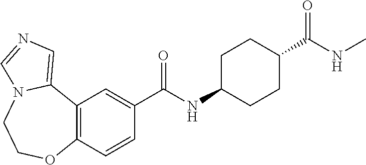 [SMILES: CNC(=O)[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and trans-4-amino-N-methyl-cyclohexanecarboxamide hydrochloride according to General Procedure E to afford the desired product N-((1r,4r)-4-(methylcarbamoyl)cyclohexyl)-5,6-dihydrobenzo[f]imidazo[1,5-d][1,4]oxazepine-10-carboxamide 17A (65 mg, 0.171 mmol, 48% yield) as a white solid. ¹H NMR (400 MHz, DMSO-D6) δ 8.26-8.16 (m, 2H), 7.77 (d, J=1.1 Hz, 1H), 7.71-7.62 (m, 2H), 7.60 (d, J=1.1 Hz, 1H), 7.03 (d, J=8.5 Hz, 1H), 4.50-4.39 (m, 4H), 3.74 (s, 1H), 2.55 (d, J=4.6 Hz, 3H), 2.11-2.00 (m, 1H), 1.92-1.84 (m, 2H), 1.81-1.73 (m, 2H), 1.51-1.28 (m, 4H). UPLC-MS (Basic 4 min): rt=1.02 min, m/z (ES+) 369.2 [M+H]⁺.

### N-(3-(Methylsulfonyl)phenyl)-5,6-dihydrobenzo[f]imidazo[1,5-d][1,4]oxazepine-10-carboxamide 18A

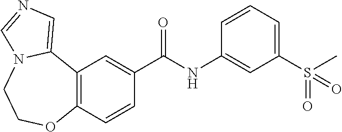 [SMILES: CS(=O)(=O)c1cccc(NC(=O)c2ccc3c(c2)-c2cncn2CCO3)c1]

Prepared from lithium 5,6-dihydrobenzo[f]imidazo[1,5-d][1,4]oxazepine-10-carboxylate 4 and 3-(methylsulfonyl) aniline according to General Procedure E to afford the desired product N-(3-(methylsulfonyl)phenyl)-5,6-dihydrobenzo[f]imidazo[1,5-d][1,4]oxazepine-10-carboxamide 18A (80 mg, 0.208 mmol, 49% yield) as a white solid. 1H NMR (400 MHz, DMSO-D6) δ 10.57 (s, 1H), 8.45-8.39 (m, 2H), 8.18-8.09 (m, 1H), 7.84-7.77 (m, 2H), 7.70-7.61 (m, 3H), 7.14 (d, J=8.5 Hz, 1H), 4.55-4.45 (m, 4H), 3.22 (s, 3H). UPLC-MS (Basic 4 min): rt=1.24 min, m/z (ES+) 384.1 [M+H]⁺.

### N-(1,1-Dioxido-2,3-dihydrobenzo[b]thiophen-5-yl)-5,6-dihydrobenzo[f]imidazo[1,5-d][1,4]oxazepine-10-carboxamide 19A

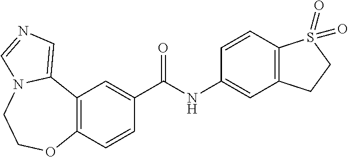 [SMILES: O=C(Nc1ccc2c(c1)CCS2(=O)=O)c1ccc2c(c1)-c1cncn1CCO2]

Prepared from lithium 5,6-dihydrobenzo[f]imidazo[1,5-d][1,4]oxazepine-10-carboxylate 4 and 1,1-dioxo-2,3-dihydrobenzothiophen-5-amine according to General Procedure E to afford the desired product N-(1,1-dioxido-2,3-dihydrobenzo[b]thiophen-5-yl)-5,6-dihydrobenzo[f]imidazo[1,5-d][1,4]oxazepine-10-carboxamide 19A (105 mg, 0.263 mmol, 62% yield) as a grey solid. ¹H NMR (400 MHz, DMSO-D6) δ 10.56 (s, 1H), 8.40 (d, J=2.3 Hz, 1H), 8.03 (d, J=1.8 Hz, 1H), 7.87 (dd, J=8.5, 1.9 Hz, 1H), 7.84-7.76 (m, 2H), 7.74 (d, J=8.5 Hz, 1H), 7.68 (d, J=1.1 Hz, 1H), 7.14 (d, J=8.6 Hz, 1H), 4.55-4.44 (m, 4H), 3.61-3.57 (m, 2H), 3.38-3.35 (m, 2H). UPLC-MS (Basic 4 min): rt=1.24 min, m/z (ES+) 396.3 [M+H]⁺.

### N-((1r,4r)-4-Methoxycyclohexyl)-5,6-dihydrobenzo[f]imidazo[1,5-d][1,4]oxazepine-10-carboxamide 20A

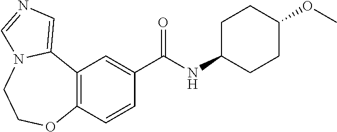 [SMILES: CO[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and trans-4-methoxycyclohexanamine according to General Procedure E to afford the desired product N-((1r,4r)-4-methoxycyclohexyl)-5,6-dihydrobenzo[f]imidazo[1,5-d][1,4]oxazepine-10-carboxamide 20A (85 mg, 0.249 mmol, 59% yield) as a white solid. 1H NMR (400 MHz, DMSO-D6) δ 8.22-8.12 (m, 2H), 7.73 (d, J=1.1 Hz, 1H), 7.62 (dd, J=8.5, 2.2 Hz, 1H), 7.57 (d, J=1.1 Hz, 1H), 7.00 (d, J=8.6 Hz, 1H), 4.47-4.35 (m, 4H), 3.71 (dtt, J=11.5, 7.9, 4.0 Hz, 1H), 3.21 (s, 3H), 3.08 (tt, J=10.4, 4.0 Hz, 1H), 2.04-1.95 (m, 2H), 1.87-1.79 (m, 2H), 1.34 (qd, J=13.1, 3.2 Hz, 2H), 1.23-1.09 (m, 2H). UPLC-MS (Basic 6 min): rt=1.87 min, m/z (ES+) 342.1 [M+H]⁺.

### N-(4-Fluoro-3-(methylsulfonyl)phenyl)-5,6-dihydrobenzo[f]imidazo[1,5-d][1,4]oxazepine-10-carboxamide 21A

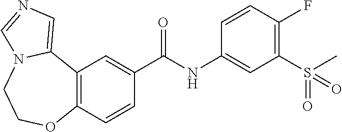 [SMILES: CS(=O)(=O)c1cc(NC(=O)c2ccc3c(c2)-c2cncn2CCO3)ccc1F]

Prepared from lithium 5,6-dihydrobenzo[f]imidazo[1,5-d][1,4]oxazepine-10-carboxylate 4 and 4-fluoro-3-(methylsulphonyl) aniline according to General Procedure E to afford the desired product N-(4-fluoro-3-(methylsulfonyl)phenyl)-5,6-dihydrobenzo[f]imidazo[1,5-d][1,4]oxazepine-10-carboxamide 21A (120 mg, 0.293 mmol, 69% yield) as an off-white solid. ¹H NMR (400 MHz, DMSO-D6) δ 10.52 (s, 1H), 8.46-8.35 (m, 2H), 8.19 (ddd, J=9.0, 4.4, 2.8 Hz, 1H), 7.83-7.76 (m, 2H), 7.65 (d, J=1.1 Hz, 1H), 7.53 (dd, J=9.9, 9.0 Hz, 1H), 7.14 (d, J=8.6 Hz, 1H), 4.56-4.45 (m, 4H), 3.34 (s, 3H). ¹⁹F NMR (376 MHz, DMSO-D6) δ-118.2-116.8 (m, F). UPLC-MS (Basic 4 min): rt=1.29 min, m/z (ES+) 402.1 [M+H]⁺.

### N-(5-(Methylsulfonyl)pyridin-3-yl)-5,6-dihydrobenzo[f]imidazo[1,5-d][1,4]oxazepine-10-carboxamide 22A

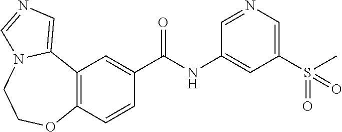 [SMILES: CS(=O)(=O)c1cncc(NC(=O)c2ccc3c(c2)-c2cncn2CCO3)c1]

Prepared from lithium 5,6-dihydrobenzo[f]imidazo[1,5-d][1,4]oxazepine-10-carboxylate 4 and 5-methylsulfonylpyridin-3-amine according to General Procedure E to afford the desired product N-(5-(methylsulfonyl)pyridin-3-yl)-5,6-dihydrobenzo[f]imidazo[1,5-d][1,4]oxazepine-10-carboxamide 22A (91 mg, 0.233 mmol, 55% yield) as an orange solid. 1H NMR (400 MHz, DMSO-D6) δ 10.73 (s, 1H), 9.50-9.00 (m, 1H), 8.82 (s, 1H), 8.77 (d, J=2.6 Hz, 1H), 8.56-8.19 (m, 1H), 7.86-7.82 (m, 1H), 7.81 (s, 1H), 7.67 (s, 1H), 7.16 (d, J=8.6 Hz, 1H), 4.51 (t, J=6.3 Hz, 2H), 4.49 (t, J=6.3 Hz, 2H), 3.35 (s, 3H). UPLC-MS (Basic 6 min): rt=1.72 min, m/z (ES+) 385.1 [M+H]⁺.

### tert-Butyl 5-(5,6-dihydrobenzo[f]imidazo[1,5-d][1,4]oxazepine-10-carboxamido)isoindoline-2-carboxylate 23A

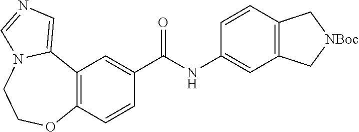 [SMILES: CC(C)(C)OC(=O)N1Cc2ccc(NC(=O)c3ccc4c(c3)-c3cncn3CCO4)cc2C1]

Prepared from lithium 5,6-dihydrobenzo[f]imidazo[1,5-d][1,4]oxazepine-10-carboxylate 4 and tert-butyl 5-aminoisoindoline-2-carboxylate according to General Procedure E to afford the desired product tert-butyl 5-(5,6-dihydrobenzo[f]imidazo[1,5-d][1,4]oxazepine-10-carboxamido)isoindoline-2-carboxylate 23A (326 mg, 0.725 mmol, 57% yield) as a white solid. ¹H NMR (400 MHz, DMSO-D6) δ 10.25 (d, J=4.2 Hz, 1H), 8.38 (dd, J=2.4, 0.9 Hz, 1H), 7.82-7.75 (m, 3H), 7.66 (d, J=1.1 Hz, 1H), 7.63 (d, J=8.3 Hz, 1H), 7.31 (dd, J=8.3, 5.7 Hz, 1H), 7.12 (d, J=8.6 Hz, 1H), 4.60 (t, J=11.9 Hz, 2H), 4.56 (t, J=11.9 Hz, 2H), 4.53-4.49 (m, 2H), 4.49-4.43 (m, 2H), 1.47 (s, 9H). UPLC-MS (Basic 4 min): rt=3.00 min, m/z (ES+) 447.2 [M+H]⁺.

### N-(Isoindolin-5-yl)-5,6-dihydrobenzo[f]imidazo[1,5-d][1,4]oxazepine-10-carboxamide hydrochloride 24A

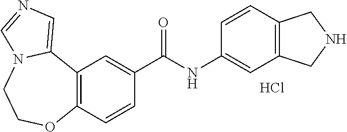 [SMILES: O=C(Nc1ccc2c(c1)CNC2)c1ccc2c(c1)-c1cncn1CCO2 ; Cl]

tert-Butyl 5-(5,6-dihydrobenzo[f]imidazo[1,5-d][1,4]oxazepine-10-carboxamido)isoindoline-2-carboxylate (275 mg, 0.616 mmol) was dissolved in hydrogen chloride solution (6.0 mL, 24.0 mmol, 4.0M in 1,4-dioxane) and stirred at room temperature for 17 h. The precipitate was collected and washed with iso-hexane, and dried under reduced pressure to afford the desired product N-(isoindolin-5-yl)-5,6-dihydrobenzo[f]imidazo[1,5-d][1,4]oxazepine-10-carboxamide hydrochloride 24A (242 mg, 0.605 mmol, 98% yield) as an off-white solid. ¹H NMR (400 MHz, DMSO-D6) δ 10.63 (s, 1H), 9.75 (s, 2H), 9.13 (s, 1H), 8.59 (d, J=2.2 Hz, 2H), 7.96 (d, J=2.0 Hz, 1H), 7.94 (d, J=2.2 Hz, 1H), 7.77 (dd, J=8.3, 1.9 Hz, 1H), 7.39 (d, J=8.3 Hz, 1H), 7.22 (d, J=8.7 Hz, 1H), 4.80-4.70 (m, 2H), 4.65-4.60 (m, 2H), 4.52 (t, J=5.6 Hz, 2H), 4.48 (t, J=5.5 Hz, 2H). UPLC-MS (Basic 4 min): rt=1.22 min, m z (ES+) 347.1 [M+H]⁺.

### N-(2-(Methylsulfonyl) isoindolin-5-yl)-5,6-dihydrobenzo[f]imidazo[1,5-d][1,4]oxazepine-10-carboxamide 25A

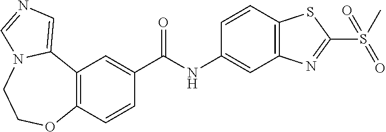 [SMILES: CS(=O)(=O)c1nc2cc(NC(=O)c3ccc4c(c3)-c3cncn3CCO4)ccc2s1]

To a suspension of N-(isoindolin-5-yl)-5,6-dihydrobenzo[f]imidazo[1,5-d][1,4]oxazepine-10-carboxamide hydrochloride (190 mg, 0.453 mmol) and triethylamine (0.28 mL, 2.02 mmol) in DCM (3 mL) was added a solution of methanesulfonyl chloride (78 mg, 0.680 mmol) in DCM (3 mL) dropwise. The resultant mixture was stirred at room temperature for 18 h. The reaction mixture was concentrated under reduced pressure and purified by column chromatography over C18 eluting with a gradient of MeCN(0.1% NH₃) (0% to 95%; v/v) in water (0.1% NH₃) to afford the desired product N-(2-(methylsulfonyl) isoindolin-5-yl)-5,6-dihydrobenzo[f]imidazo[1,5-d][1,4]oxazepine-10-carboxamide 25A (27 mg, 0.0622 mmol, 14% yield) as an off-white solid. ¹H NMR (400 MHz, DMSO-D6) δ 10.28 (s, 1H), 8.39 (d, J=2.2 Hz, 1H), 7.81 (d, J=1.8 Hz, 1H), 7.79 (d, J=1.1 Hz, 1H), 7.77 (dd, J=8.6, 2.2 Hz, 1H), 7.66 (d, J=1.1 Hz, 1H), 7.64 (d, J=2.0 Hz, 1H), 7.32 (d, J=8.3 Hz, 1H), 7.12 (d, J=8.6 Hz, 1H), 4.65 (s, 2H), 4.61 (d, J=2.2 Hz, 2H), 4.55-4.49 (m, 2H), 4.49-4.42 (m, 2H), 2.99 (s, 3H). UPLC-MS (Basic 6 min): rt=2.05 min, m/z (ES+) 425.4 [M+H]⁺.

### (R)—N-(Tetrahydro-2H-pyran-3-yl)-6,7-dihydro-5H-benzo[b]imidazo[5,1-d][1,5]oxazocine-11-carboxamide 26A

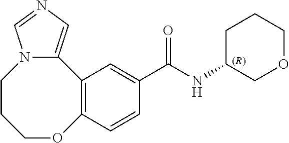

Prepared from lithium 6,7-dihydro-5H-benzo[b]imidazo[5,1-d][1,5]oxazocine-11-carboxylate 20 and (R)-tetrahydro-2H-pyran-3-amine hydrochloride according to General Procedure E to afford the desired product (R)—N-(tetrahydro-2H-pyran-3-yl)-6,7-dihydro-5H-benzo[b]imidazo[5,1-d][1,5]oxazocine-11-carboxamide 26A (19 mg, 0.057 mmol, 24% yield) as a white solid. ¹H NMR (400 MHz, DMSO-D6) δ 8.18 (d, J=7.8 Hz, 1H), 7.88-7.79 (m, 2H), 7.76 (d, J=1.1 Hz, 1H), 7.16-7.10 (m, 2H), 4.13 (dd, J=6.1, 4.7 Hz, 2H), 4.03 (dd, J=6.7, 4.5 Hz, 2H), 3.89-3.87 (m, 1H), 3.83-3.72 (m, 2H), 3.27-3.24 (m, 1H), 3.14 (dd, J=10.7, 9.5 Hz, 1H), 1.96-1.92 (m, 3H), 1.69-1.68 (m, 1H), 1.66-1.51 (m, 2H). UPLC-MS (Basic 4 min): rt=1.17 min, m/z (ES+) 328.1 [M+H]⁺.

### Methyl 4-((2-methoxyethyl)amino)imidazo[1,5-a]quinoxaline-8-carboxylate

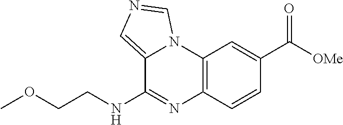 [SMILES: COCCNc1nc2ccc(C(=O)OC)cc2n2cncc12]

To methyl 4-chloroimidazo[1,5-a]quinoxaline-8-carboxylate 21 (77 mg, 0.573 mmol) in MeCN(1.1 mL) was added 2-methoxyethylamine (0.26 mL, 2.94 mmol) and the reaction stirred at room temperature for 18 h. This was concentrated under reduced pressure to afford the desired product methyl 4-((2-methoxyethyl)amino)imidazo[1,5-a]quinoxaline-8-carboxylate (75 mg, 0.192 mmol, 65% yield) as a brown solid. ¹H NMR (400 MHz, DMSO-D6) δ 9.26 (d, J=0.7 Hz, 1H), 8.73 (dd, J=14.4, 1.8 Hz, 1H), 8.26 (t, J=5.6 Hz, 1H), 7.99 (d, J=0.7 Hz, 1H), 7.97-7.86 (m, 1H), 7.53 (d, J=8.5 Hz, 1H), 3.89 (d, J=3.9 Hz, 3H), 3.74 (q, J=5.6 Hz, 2H), 3.60 (t, J=5.6 Hz, 2H), 3.30 (s, 3H). UPLC-MS (Basic 2 min): rt=0.91 min, m/z (ES+) 301.3 [M+H]⁺.

### Lithium 4-((2-methoxyethyl)amino)imidazo[1,5-a]quinoxaline-8-carboxylate

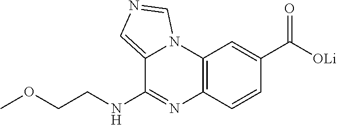

Lithium hydroxide (49 mg, 1.17 mmol) was added to a solution of methyl 4-((2-methoxyethyl)amino)imidazo[1,5-a]quinoxaline-8-carboxylate (70 mg, 0.233 mmol) in THF (1.1 mL) and water (1.1 mL) and this was stirred at room temperature for 2 h. This was concentrated under reduced pressure and used directly in the next step. ¹H NMR (400 MHz, DMSO-D6) δ 8.99 (s, 1H), 8.51 (d, J=1.7 Hz, 1H), 7.92-7.82 (m, 2H), 7.38-7.28 (m, 1H), 3.70 (t, J=5.8 Hz, 2H), 3.59 (t, J=5.7 Hz, 2H), 3.30 (s, 3H). NH not observed. UPLC-MS (Basic 2 min): rt=0.60 min, m/z (ES+) 287.2 [M+H]⁺.

### (R)-4-((2-Methoxyethyl)amino)-N-(tetrahydro-2H-pyran-3-yl)imidazo[1,5-a]quinoxaline-8-carboxamide 27A

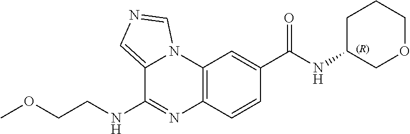

Prepared from lithium 4-((2-methoxyethyl)amino)imidazo[1,5-a]quinoxaline-8-carboxylate and (R)-tetrahydro-2H-pyran-3-amine hydrochloride according to General Procedure E, using DMSO as a co-solvent, to afford the desired product (R)-4-((2-methoxyethyl)amino)-N-(tetrahydro-2H-pyran-3-yl)imidazo[1,5-a]quinoxaline-8-carboxamide 27A (47 mg, 0.122 mmol, 51% yield) as an off-white solid. ¹H NMR (400 MHz, DMSO-D6) δ 9.05 (d, J=0.8 Hz, 1H), 8.56 (d, J=2.0 Hz, 1H), 8.14 (d, J=7.7 Hz, 1H), 8.06 (t, J=5.6 Hz, 1H), 7.93 (d, J=0.7 Hz, 1H), 7.80 (dd, J=8.5, 1.9 Hz, 1H), 7.46 (d, J=8.5 Hz, 1H), 3.98-3.77 (m, 2H), 3.77-3.64 (m, 3H), 3.56 (t, J=5.6 Hz, 2H), 3.26 (s, 3H), 3.16 (dd, J=10.8, 9.3 Hz, 1H), 1.93 (d, J=10.4 Hz, 1H), 1.73-1.52 (m, 3H). 1H not observed. UPLC-MS (Basic 4 min): rt=1.17 min, m/z (ES+) 370.1 [M+H]⁺.

### Methyl 4-(methylamino)imidazo[1,5-a]quinoxaline-8-carboxylate

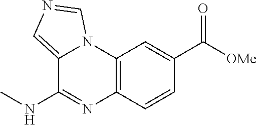 [SMILES: CNc1nc2ccc(C(=O)OC)cc2n2cncc12]

To methyl 4-chloroimidazo[1,5-a]quinoxaline-8-carboxylate 21 (150 mg, 0.573 mmol) in MeCN(2 mL) was added methylamine solution (2.0 M in THF, 4.0 mL, 111 mmol) and the reaction stirred at room temperature for 18 h. This was concentrated under reduced pressure and purified by column chromatography over silica eluting with a gradient of methanol (0% to 20%; v/v) in DCM to afford the desired product methyl 4-(methylamino)imidazo[1,5-a]quinoxaline-8-carboxylate (145 mg, 0.430 mmol, 75% yield) as a brown solid. ¹H NMR (400 MHz, DMSO-D6) δ 9.25 (d, J=0.7 Hz, 1H), 8.72 (dd, J=15.1, 1.8 Hz, 1H), 8.22 (d, J=4.9 Hz, 1H), 7.92-7.86 (m, 2H), 7.54 (d, J=8.5 Hz, 1H), 3.89 (s, 3H), 3.04 (d, J=4.6 Hz, 3H). UPLC-MS (Basic 2 min): rt=0.87 min, m/z (ES+) 257.1 [M+H]⁺.

### Lithium 4-(methylamino)imidazo[1,5-a]quinoxaline-8-carboxylate

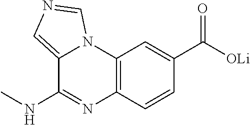

Lithium hydroxide (458 mg, 10.9 mmol) was added to a solution of methyl 4-(methylamino)imidazo[1,5-a]quinoxaline-8-carboxylate (140 mg, 0.546 mmol) in THF (1.1 mL) and water (1.1 mL) and this was stirred at room temperature for 18 h. This was concentrated under reduced pressure and used directly in the next step. ¹H NMR (400 MHz, DMSO-D6) δ 9.01 (d, J=0.7 Hz, 1H), 8.51 (d, J=1.7 Hz, 1H), 7.91-7.75 (m, 3H), 7.38 (d, J=8.3 Hz, 1H), 3.01 (d, J=3.9 Hz, 3H).

### (R)-4-(Methylamino)-N-(tetrahydro-2H-pyran-3-yl)imidazo[1,5-a]quinoxaline-8-carboxamide 28A

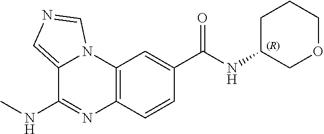

Prepared from lithium 4-(methylamino)imidazo[1,5-a]quinoxaline-8-carboxylate and (R)-tetrahydro-2H-pyran-3-amine hydrochloride according to General Procedure E, using DMSO as the reaction solvent, to afford the desired product (R)-4-(methylamino)-N-(tetrahydro-2H-pyran-3-yl)imidazo[1,5-a]quinoxaline-8-carboxamide 28A (42 mg, 0.128 mmol, 23% yield) as a white solid. ¹H NMR (400 MHz, DMSO-D6) δ 9.05 (d, J=0.7 Hz, 1H), 8.56 (d, J=1.9 Hz, 1H), 8.14 (d, J=7.7 Hz, 1H), 8.00 (q, J=4.6 Hz, 1H), 7.85-7.76 (m, 2H), 7.48 (d, J=8.5 Hz, 1H), 3.95-3.79 (m, 1H), 3.83-3.70 (m, 2H), 3.16 (dd, J=10.7, 9.3 Hz, 1H), 3.00 (d, J=4.6 Hz, 3H), 1.93 (d, J=10.6 Hz, 1H), 1.73-1.52 (m, 3H). 1H not observed. UPLC-MS (Basic 4 min): rt=1.07 min, m/z (ES+) 326.3 [M+H]⁺.

### N-((1r,4r)-4-((2-Methoxyethyl) sulfonamido)cyclohexyl)-5,6-dihydrobenzo[f]imidazo[1,5-d][1,4]oxazepine-10-carboxamide 29A

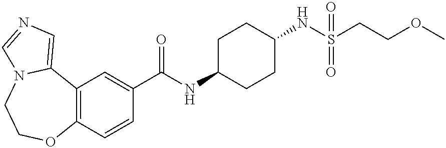 [SMILES: COCCS(=O)(=O)N[C@H]1CC[C@H](NC(=O)c2ccc3c(c2)-c2cncn2CCO3)CC1]

Triethylamine (0.15 mL, 1.10 mmol) was added to a suspension of N-((1r,4r)-4-aminocyclohexyl)-5,6-dihydrobenzo[f]imidazo[1,5-d][1,4]oxazepine-10-carboxamide hydrochloride 23 (100 mg, 0.276 mmol) in DCM (12 mL) and stirred at room temperature for 5 mins. A solution of 2-methoxy-1-ethanesulfonyl chloride (66 mg, 0.413 mmol) in DCM (2 mL) was added dropwise to the mixture and the resulting mixture was stirred at room temperature for 5 days. This was concentrated under reduced pressure and the resulting white solid was suspended in NMP (8 mL) and the resulting mixture was stirred at room temperature for 3 h. Further triethylamine (0.15 mL, 1.10 mmol) and 2-methoxy-1-ethanesulfonyl chloride (66 mg, 0.413 mmol) as a solution in DCM (0.5 mL) were added and the resulting solution was stirred at room temperature for 22 h. This was purified directly by column chromatography over C18 eluting with a gradient of MeCN(0.1% NH₃) (5% to 80%; v/v) in water (0.1% NH₃) to give crude product. The solid was suspended in diethyl ether (10 mL) and stirred at room temperature for 30 mins. The white suspension was filtered and dried to afford the desired product N-((1r,4r)-4-((2-methoxyethyl) sulfonamido)cyclohexyl)-5,6-dihydrobenzo[f]imidazo[1,5-d][1,4]oxazepine-10-carboxamide 29A (77 mg, 0.172 mmol, 62% yield) as a white solid. ¹H NMR (400 MHz, DMSO-D6) δ 8.22 (d, J=2.2 Hz, 1H), 8.14 (d, J=7.7 Hz, 1H), 7.75 (d, J=1.1 Hz, 1H), 7.66 (dd, J=8.6, 2.2 Hz, 1H), 7.59 (d, J=1.1 Hz, 1H), 7.03 (d, J=8.5 Hz, 2H), 4.51-4.39 (m, 4H), 3.77-3.63 (m, 3H), 3.33-3.26 (m, 5H), 3.16-3.05 (m, 1H), 1.91 (dd, J=30.6, 11.8 Hz, 4H), 1.50-1.28 (m, 4H). UPLC-MS (Basic 4 min): rt=1.12 min, m/z (ES+) 449.2 [M+H]⁺.

### N-(2,3-Dihydro-[1,4]dioxino[2,3-b]pyridin-6-yl)-5,6-dihydrobenzo[f]imidazo[1,5-d][1,4]oxazepine-10-carboxamide 30A

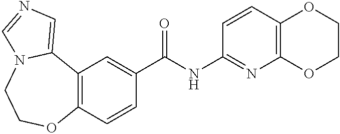 [SMILES: O=C(Nc1ccc2c(n1)OCCO2)c1ccc2c(c1)-c1cncn1CCO2]

Prepared from lithium 5,6-dihydrobenzo[f]imidazo[1,5-d][1,4]oxazepine-10-carboxylate 4 and 2,3-dihydro-[1,4]dioxino[2,3-b]pyridin-6-amine according to General Procedure E to afford the desired product N-(2,3-dihydro-[1,4]dioxino[2,3-b]pyridin-6-yl)-5,6-dihydrobenzo[f]imidazo[1,5-d][1,4]oxazepine-10-carboxamide 30A (57 mg, 0.156 mmol, 37% yield) as an off-white solid. ¹H NMR (400 MHz, DMSO-D6) δ 10.71 (s, 1H), 8.49 (d, J=2.3 Hz, 1H), 7.80 (dd, J=8.6, 2.2 Hz, 1H), 7.78-7.73 (m, 3H), 7.35 (d, J=8.5 Hz, 1H), 7.07 (d, J=8.7 Hz, 1H), 4.53-4.39 (m, 6H), 4.28-4.22 (m, 2H). UPLC-MS (Basic 6 min): rt=2.07 min, m/z (ES+) 365.1 [M+H]⁺.

### N-(2,3-Dihydro-[1,4]dioxino[2,3-b]pyridin-7-yl)-5,6-dihydrobenzo[f]imidazo[1,5-d][1,4]oxazepine-10-carboxamide 31A

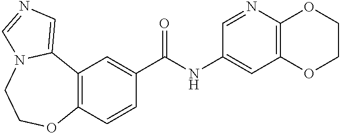 [SMILES: O=C(Nc1cnc2c(c1)OCCO2)c1ccc2c(c1)-c1cncn1CCO2]

Prepared from lithium 5,6-dihydrobenzo[f]imidazo[1,5-d][1,4]oxazepine-10-carboxylate 4 and 2,3-dihydro-[1,4]dioxino[2,3-b]pyridin-7-amine dihydrochloride according to General Procedure E to afford the desired product N-(2,3-dihydro-[1,4]dioxino[2,3-b]pyridin-7-yl)-5,6-dihydrobenzo[f]imidazo[1,5-d][1,4]oxazepine-10-carboxamide 31A (20 mg, 0.0548 mmol, 17% yield) as an off-white solid. ¹H NMR (400 MHz, DMSO-D6) δ 10.25 (s, 1H), 8.38 (d, J=2.2 Hz, 1H), 8.10 (d, J=2.3 Hz, 1H), 7.79 (d, J=1.0 Hz, 1H), 7.78-7.73 (m, 2H), 7.66 (d, J=1.1 Hz, 1H), 7.12 (d, J=8.6 Hz, 1H), 4.54-4.43 (m, 4H), 4.42-4.36 (m, 2H), 4.30-4.23 (m, 2H). UPLC-MS (Basic 6 min): rt=1.80 min, m/z (ES+) 365.1 [M+H]⁺.

### N-(2,3-Dihydrobenzofuran-6-yl)-5,6-dihydrobenzo[f]imidazo[1,5-d][1,4]oxazepine-10-carboxamide 32A

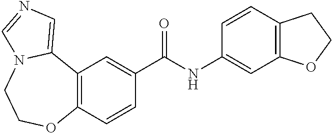 [SMILES: O=C(Nc1ccc2c(c1)OCC2)c1ccc2c(c1)-c1cncn1CCO2]

Prepared from lithium 5,6-dihydrobenzo[f]imidazo[1,5-d][1,4]oxazepine-10-carboxylate 4 and 2,3-dihydro-6-benzofuranamine according to General Procedure E to afford the desired product N-(2,3-dihydrobenzofuran-6-yl)-5,6-dihydrobenzo[f]imidazo[1,5-d][1,4]oxazepine-10-carboxamide 32A (107 mg, 0.298 mmol, 56% yield) as a beige solid. 1H NMR (400 MHz, DMSO-D6) δ 10.11 (s, 1H), 8.35 (d, J=2.2 Hz, 1H), 7.79 (s, 1H), 7.75 (dd, J=8.6, 2.2 Hz, 1H), 7.66 (s, 1H), 7.30 (s, 1H), 7.23-7.14 (m, 2H), 7.11 (d, J=8.6 Hz, 1H), 4.58-4.43 (m, 6H), 3.14 (t, J=8.6 Hz, 2H). UPLC-MS (Basic 6 min): rt=2.29 min, m/z (ES+) 348.1 [M+H]⁺.

### N-(1,3-Dihydroisobenzofuran-5-yl)-5,6-dihydrobenzo[f]imidazo[1,5-d][1,4]oxazepine-10-carboxamide 33A

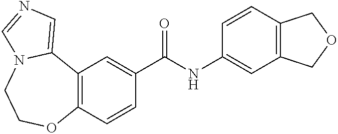 [SMILES: O=C(Nc1ccc2c(c1)COC2)c1ccc2c(c1)-c1cncn1CCO2]

Prepared from lithium 5,6-dihydrobenzo[f]imidazo[1,5-d][1,4]oxazepine-10-carboxylate 4 and 1,3-dihydroisobenzofuran-5-amine according to General Procedure E to afford the desired product N-(1,3-dihydroisobenzofuran-5-yl)-5,6-dihydrobenzo[f]imidazo[1,5-d][1,4]oxazepine-10-carboxamide 33A (91 mg, 0.260 mmol, 61% yield) as a brown solid. 1H NMR (400 MHz, DMSO-D6) δ 10.25 (s, 1H), 8.38 (d, J=2.3 Hz, 1H), 7.83-7.73 (m, 3H), 7.67 (s, 1H), 7.62 (dd, J=8.2, 1.9 Hz, 1H), 7.29 (d, J=8.2 Hz, 1H), 7.12 (d, J=8.6 Hz, 1H), 5.04-4.96 (m, 4H), 4.54-4.43 (m, 4H). UPLC-MS (Basic 6 min): rt=2.05 min, m/z (ES+) 348.1 [M+H]⁺.

### N-(1H-Benzo [d]imidazol-6-yl)-5,6-dihydrobenzo[f]imidazo[1,5-d][1,4]oxazepine-10-carboxamide 34A

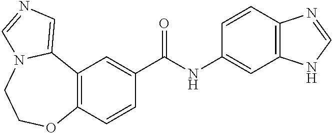 [SMILES: O=C(Nc1ccc2nc[nH]c2c1)c1ccc2c(c1)-c1cncn1CCO2]

Prepared from lithium 5,6-dihydrobenzo[f]imidazo[1,5-d][1,4]oxazepine-10-carboxylate 4 and 5-aminobenzimidazole according to General Procedure E to afford the desired product N-(1H-benzo[d]imidazol-6-yl)-5,6-dihydrobenzo[f]imidazo[1,5-d][1,4]oxazepine-10-carboxamide 34A (25 mg, 0.0685 mmol, 16% yield) as a beige solid. ¹H NMR (400 MHz, DMSO-D6) δ 12.35 (s, 1H), 10.21 (s, 1H), 8.41 (d, J=2.3 Hz, 1H), 8.17 (s, 1H), 8.15 (s, 1H), 7.80 (ddd, J=4.9, 2.2, 2.2 Hz, 2H), 7.68 (d, J=1.1 Hz, 1H), 7.56 (d, J=8.7 Hz, 1H), 7.48 (d, J=8.6 Hz, 1H), 7.12 (d, J=8.5 Hz, 1H), 4.55-4.44 (m, 4H). UPLC-MS (Basic 6 min): rt=1.53 min, m/z (ES+) 346.0 [M+H]⁺.

### Methyl 4-(dimethylamino)imidazo[1,5-a]quinoxaline-8-carboxylate

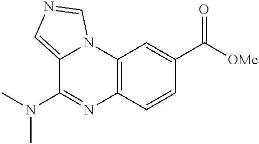 [SMILES: COC(=O)c1ccc2nc(N(C)C)c3cncn3c2c1]

To methyl 4-chloroimidazo[1,5-a]quinoxaline-8-carboxylate 21 (75 mg, 0.287 mmol) in MeCN(2 mL) was added dimethylamine solution (2.0 M in MeOH, 1.0 mL, 111 mmol) and the reaction stirred at room temperature for 18 h. This was concentrated under reduced pressure to afford the desired product methyl 4-(dimethylamino)imidazo[1,5-a]quinoxaline-8-carboxylate (75 mg, 0.236 mmol, 82% yield) as a brown solid. ¹H NMR (400 MHz, DMSO-D6) 9.30 (s, 1H), 8.66 (d, J=1.9 Hz, 1H), 7.96 (d, J=0.6 Hz, 1H), 7.87-7.81 (m, 1H), 7.43 (d, J=8.5 Hz, 1H), 3.85 (s, 3H), 3.38 (s, 6H). UPLC-MS (Basic 2 min): rt=0.96 min, m/z (ES+) 271.1 [M+H]⁺.

### Lithium 4-(dimethylamino)imidazo[1,5-a]quinoxaline-8-carboxylate

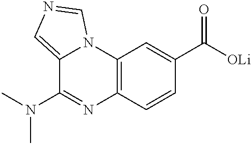

Lithium hydroxide (58 mg, 1.39 mmol) was added to a solution of methyl 4-(dimethylamino)imidazo[1,5-a]quinoxaline-8-carboxylate (75 mg, 0.277 mmol) in THF (1.1 mL) and water (1.1 mL) and this was stirred at room temperature for 18 h. This was concentrated under reduced pressure and used directly in the next step without characterisation.

### (R)-4-(Dimethylamino)-N-(tetrahydro-2H-pyran-3-yl)imidazo[1,5-a]quinoxaline-8-carboxamide 35A

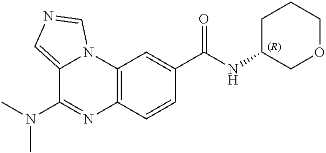

Prepared from lithium 4-(dimethylamino)imidazo[1,5-a]quinoxaline-8-carboxylate and (R)-tetrahydro-2H-pyran-3-amine hydrochloride according to General Procedure E to afford the desired product (R)-4-(dimethylamino)-N-(tetrahydro-2H-pyran-3-yl)imidazo[1,5-a]quinoxaline-8-carboxamide 35A (5.6 mg, 0.0162 mmol, 6% yield) as a white solid. ¹H NMR (400 MHz, DMSO-D6) δ 9.18 (d, J=1.5 Hz, 1H), 8.59 (d, J=1.9 Hz, 1H), 8.16 (d, J=7.5 Hz, 1H), 7.99 (d, J=1.4 Hz, 1H), 7.83 (dt, J=8.4, 1.6 Hz, 1H), 7.45 (dd, J=8.5, 1.3 Hz, 1H), 3.95 (s, 1H), 3.85 (dd, J=10.7, 4.2 Hz, 1H), 3.78 (d, J=11.1 Hz, 1H), 3.41 (s, 6H), 3.20 (dd, J=10.8, 9.3 Hz, 1H), 2.0-1.93 (m, 1H), 1.76-1.66 (m, 1H), 1.65-1.53 (m, 2H). 1H not observed. UPLC-MS (Basic 4 min): rt=1.22 min, m/z (ES+) 340.2 [M+H]⁺.

### N-(1-Methyl-2-oxo-1,2-dihydropyridin-4-yl)-5,6-dihydrobenzo[f]imidazo[1,5-d][1,4]oxazepine-10-carboxamide 36A

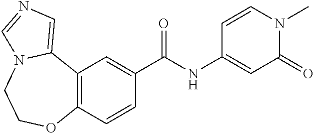 [SMILES: Cn1ccc(NC(=O)c2ccc3c(c2)-c2cncn2CCO3)cc1=O]

Prepared from lithium 5,6-dihydrobenzo[f]imidazo[1,5-d][1,4]oxazepine-10-carboxylate 4 and 4-amino-1-methylpyridin-2 (1H)-one hydrochloride according to General Procedure E, with the reaction heated at 60° C. for 18 h following 18 h at rt, to afford the desired product N-(1-methyl-2-oxo-1,2-dihydropyridin-4-yl)-5,6-dihydrobenzo[f]imidazo[1,5-d][1,4]oxazepine-10-carboxamide 36A (18 mg, 0.0535 mmol, 6% yield) as a beige solid. 1H NMR (400 MHz, DMSO-D6) δ 10.19 (s, 1H), 8.34 (d, J=2.3 Hz, 1H), 7.80-7.71 (m, 2H), 7.68-7.58 (m, 2H), 7.12 (d, J=8.6 Hz, 1H), 6.94 (d, J=2.3 Hz, 1H), 6.63 (dd, J=7.4, 2.4 Hz, 1H), 4.54-4.45 (m, 4H), 3.38 (s, 3H). UPLC-MS (Basic 4 min): rt=1.00 min, m/z (ES+) 337.1 [M+H]⁺.

### N-((1r,4r)-4-(Propylsulfonamido)cyclohexyl)-5,6-dihydrobenzo[f]imidazo[1,5-d][1,4]oxazepine-10-carboxamide 37A

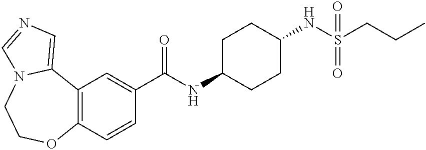 [SMILES: CCCS(=O)(=O)N[C@H]1CC[C@H](NC(=O)c2ccc3c(c2)-c2cncn2CCO3)CC1]

Triethylamine (0.15 mL, 1.10 mmol) was added to a suspension of N-((1r,4r)-4-aminocyclohexyl)-5,6-dihydrobenzo[f]imidazo[1,5-d][1,4]oxazepine-10-carboxamide hydrochloride 23 (100 mg, 0.276 mmol) in DCM (12 mL) and stirred at room temperature for 5 mins. A solution of propanesulfonyl chloride (63 mg, 0.441 mmol) in DCM (2 mL) was added dropwise to the mixture and the resulting mixture was stirred at room temperature for 6 days. This was concentrated under reduced pressure and the resulting white solid was suspended in NMP (8 mL) and the resulting mixture was stirred at room temperature for 3 h. Further triethylamine (0.15 mL, 1.10 mmol) and propanesulfonyl chloride (63 mg, 0.441 mmol) as a solution in DCM (0.5 mL) were added and the resulting solution was stirred at room temperature for 22 h. This was purified directly by column chromatography over C18 eluting with a gradient of MeCN(0.1% NH₃) (5% to 80%; v/v) in water (0.1% NH₃) to give crude product. This was purified by prep HPLC to afford the desired product N-((1r,4r)-4-(propylsulfonamido)cyclohexyl)-5,6-dihydrobenzo[f]imidazo[1,5-d][1,4]oxazepine-10-carboxamide 37A (53 mg, 0.123 mmol, 44% yield) as a white solid. ¹H NMR (400 MHz, DMSO-D6) δ 8.22 (d, J=2.2 Hz, 1H), 8.14 (d, J=7.7 Hz, 1H), 7.75 (d, J=1.1 Hz, 1H), 7.65 (dd, J=8.6, 2.2 Hz, 1H), 7.59 (d, J=1.1 Hz, 1H), 7.03 (d, J=8.5 Hz, 1H), 6.96 (d, J=6.5 Hz, 1H), 4.51-4.39 (m, 4H), 3.78-3.64 (m, 1H), 3.16-3.02 (m, 1H), 3.02-2.93 (m, 2H), 1.97-1.84 (m, 4H), 1.76-1.62 (m, 2H), 1.49-1.29 (m, 4H), 0.99 (t, J=7.4 Hz, 3H). UPLC-MS (Basic 4 min): rt=1.19 min, m/z (ES+) 433.3 [M+H]⁺.

### (R)-6,6-Dimethyl-N-(tetrahydro-2H-pyran-3-yl)-5,6-dihydrobenzo[f]imidazo[1,5-d][1,4]oxazepine-10-carboxamide 38A

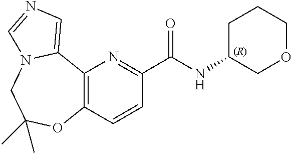

Prepared from lithium 6,6-dimethyl-5,6-dihydrobenzo[f]imidazo[1,5-d][1,4]oxazepine-10-carboxylate 27 and (R)-tetrahydro-2H-pyran-3-amine hydrochloride according to General Procedure E to afford the desired product (R)-6,6-dimethyl-N-(tetrahydro-2H-pyran-3-yl)-5,6-dihydrobenzo[f]imidazo[1,5-d][1,4]oxazepine-10-carboxamide 38A (10 mg, 0.0286 mmol, 29% yield) as a white solid. ¹H NMR (400 MHz, DMSO-D6) δ 8.21 (d, J=7.8 Hz, 1H), 8.02 (d, J=2.3 Hz, 1H), 7.80 (d, J=1.0 Hz, 1H), 7.77 (dd, J=8.4, 2.2 Hz, 1H), 7.33 (d, J=1.0 Hz, 1H), 7.07 (d, J=8.4 Hz, 1H), 4.11 (s, 2H), 3.96-3.86 (m, 1H), 3.82-3.73 (m, 2H), 3.30-3.25 (m, 1H), 3.15 (dd, J=10.7, 9.6 Hz, 1H), 1.96-1.89 (m, 1H), 1.73-1.53 (m, 3H), 1.32 (s, 6H). UPLC-MS (Basic 4 min): rt=1.25 min, m/z (ES+) 342.1 [M+H]⁺.

### N-(Benzo[d]oxazol-6-yl)-5,6-dihydrobenzo[f]imidazo[1,5-d][1,4]oxazepine-10-carboxamide 39A

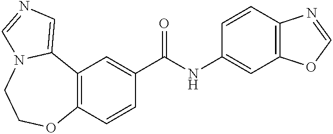 [SMILES: O=C(Nc1ccc2ncoc2c1)c1ccc2c(c1)-c1cncn1CCO2]

Prepared from lithium 5,6-dihydrobenzo[f]imidazo[1,5-d][1,4]oxazepine-10-carboxylate 4 and benzo[d]oxazol-6-amine according to General Procedure E to afford the desired product N-(benzo[d]oxazol-6-yl)-5,6-dihydrobenzo[f]imidazo[1,5-d][1,4]oxazepine-10-carboxamide 39A (98 mg, 0.281 mmol, 74% yield) as a beige solid. ¹H NMR (400 MHz, DMSO-D6) δ 10.46 (s, 1H), 8.69 (s, 1H), 8.41 (d, J=2.3 Hz, 1H), 8.36 (d, J=1.8 Hz, 1H), 7.82-7.76 (m, 3H), 7.71-7.66 (m, 2H), 7.14 (d, J=8.6 Hz, 1H), 4.55-4.44 (m, 4H). UPLC-MS (Basic 4 min): rt=1.24 min, m/z (ES+) 347.0 [M+H]⁺.

### N-(Thiazolo[5,4-b]pyridin-6-yl)-5,6-dihydrobenzo[f]imidazo[1,5-d][1,4]oxazepine-10-carboxamide 40A

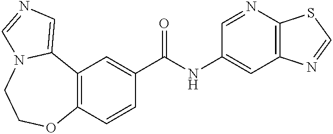 [SMILES: O=C(Nc1cnc2scnc2c1)c1ccc2c(c1)-c1cncn1CCO2]

Prepared from lithium 5,6-dihydrobenzo[f]imidazo[1,5-d][1,4]oxazepine-10-carboxylate 4 and thiazolo[5,4-b]pyridin-6-amine according to General Procedure E to afford the desired product N-(thiazolo[5,4-b]pyridin-6-yl)-5,6-dihydrobenzo[f]imidazo[1,5-d][1,4]oxazepine-10-carboxamide 40A (43 mg, 0.117 mmol, 62% yield) as a yellow solid. 1H NMR (400 MHz, DMSO-D6) δ 10.67 (s, 1H), 9.57 (s, 1H), 9.02 (d, J=2.3 Hz, 1H), 8.92 (d, J=2.3 Hz, 1H), 8.46 (d, J=2.3 Hz, 1H), 7.87-7.79 (m, 2H), 7.70 (d, J=1.2 Hz, 1H), 7.16 (d, J=8.5 Hz, 1H), 4.55-4.46 (m, 4H). UPLC-MS (Basic 4 min): rt=1.20 min, m/z (ES+) 364.0

### N-(Benzo[d]thiazol-6-yl)-5,6-dihydrobenzo[f]imidazo[1,5-d][1,4]oxazepine-10-carboxamide 41A

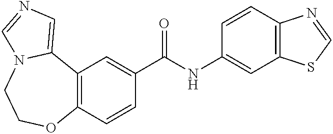 [SMILES: O=C(Nc1ccc2ncsc2c1)c1ccc2c(c1)-c1cncn1CCO2]

Prepared from lithium 5,6-dihydrobenzo[f]imidazo[1,5-d][1,4]oxazepine-10-carboxylate 4 and 6-aminobenzothiazole according to General Procedure E to afford the desired product N-(benzo[d]thiazol-6-yl)-5,6-dihydrobenzo[f]imidazo[1,5-d][1,4]oxazepine-10-carboxamide 41A (77 mg, 0.209 mmol, 55% yield) as a beige solid. ¹H NMR (400 MHz, DMSO-D6) δ 10.47 (s, 1H), 9.30 (s, 1H), 8.68 (d, J=2.0 Hz, 1H), 8.43 (d, J=2.3 Hz, 1H), 8.08 (d, J=8.8 Hz, 1H), 7.87-7.77 (m, 3H), 7.68 (s, 1H), 7.14 (d, J=8.6 Hz, 1H), 4.55-4.44 (m, 4H). UPLC-MS (Basic 4 min): rt=1.30 min, m/z (ES+) 363.0 [M+H]⁺.

### (R)—N-(tetrahydro-2H-pyran-3-yl)-6,7-dihydroimidazo[1,5-d]pyrido[3,2-b][1,4]oxazepine-2-carboxamide 42A

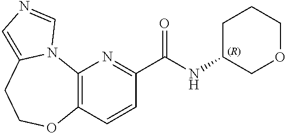

A suspension of (R)-5-(2-(/H-imidazol-5-yl)ethoxy)-6-bromo-N-(tetrahydro-2H-pyran-3-yl) picolinamide 29 (35 mg, 0.0885 mmol) and cesium carbonate (144 mg, 0.443 mmol) in NMP (1 mL) was heated to 100° C. for 1.5 h. The reaction mixture was dissolved in DCM: MeOH (9:1, 20 mL) and washed with water (2×10 mL). The organic fraction was dried (Na₂SO₄) and concentrated under reduced pressure. This was purified by prep HPLC to afford the desired product (R)—N-(tetrahydro-2H-pyran-3-yl)-6,7-dihydroimidazo[1,5-d]pyrido[3,2-b][1,4]oxazepine-2-carboxamide 42A (1.2 mg, 0.00376 mmol, 4% yield) as a white solid. 1H NMR (400 MHz, Acetonitrile-D3) δ 8.51 (d, J=1.1 Hz, 1H), 7.99 (d, J=8.2 Hz, 1H), 7.79 (s, 1H), 7.66 (d, J=8.3 Hz, 1H), 6.90 (q, J=1.0 Hz, 1H), 4.50-4.43 (m, 2H), 4.03-4.01 (m, 1H), 3.85-3.81 (m, 1H), 3.76-3.72 (m, 1H), 3.50-3.33 (m, 2H), 3.20-3.17 (m, 2H), 1.82-1.57 (m, 3H). 1H not observed. UPLC-MS (Basic 4 min): rt=1.16 min, m/z (ES+) 315.3 [M+H]⁺.

### (Z)—N—((R)-Tetrahydro-2H-pyran-3-yl)-5,6-dihydro-1,4-(metheno)pyrido[3,2-b][1,4,6]oxadiazonine-10-carboxamide 43A

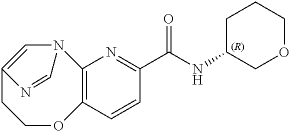

Also isolated by prep HPLC from the above reaction was the other desired product (Z)—N—((R)-tetrahydro-2H-pyran-3-yl)-5,6-dihydro-1,4-(metheno)pyrido[3,2-b][1,4,6]oxadiazonine-10-carboxamide 43A (1.2 mg, 0.00376 mmol, 4% yield) as a white solid. ¹H NMR (400 MHz, Acetonitrile-D3) δ 8.03 (dd, J=24.9, 8.0 Hz, 2H), 7.94 (s, 1H), 7.87 (d, J=1.0 Hz, 1H), 6.96 (d, J=0.9 Hz, 1H), 4.62 (t, J=6.1 Hz, 2H), 4.12-3.93 (m, 1H), 3.83-3.80 (m, 1H), 3.74-3.63 (m, 1H), 3.60-3.57 (m, 1H), 3.46-3.43 (m, 1H), 3.09-3.06 (m, 2H), 1.83-1.67 (m, 1H), 1.67-1.57 (m, 1H). 2H not observed. UPLC-MS (Basic 4 min): rt=1.07 min, m z (ES+) 315.0 [M+H]⁺.

### N-(2-Methoxybenzo[d]thiazol-5-yl)-5,6-dihydrobenzo[f]imidazo[1,5-d][1,4]oxazepine-10-carboxamide 44A

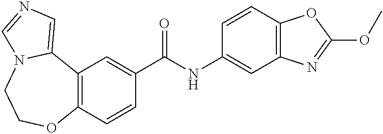 [SMILES: COc1nc2cc(NC(=O)c3ccc4c(c3)-c3cncn3CCO4)ccc2o1]

To a suspension of N-(2-chlorobenzo[d]thiazol-5-yl)-5,6-dihydrobenzo[f]imidazo[1,5-d][1,4]oxazepine-10-carboxamide 30 (50 mg, 0.117 mmol) in THF (2.5 mL) was added sodium methoxide solution (2M in MeOH, 0.060 mL, 0.262 mmol). The resulting reaction mixture was stirred at room temperature for 18 h. The reaction mixture was concentrated under reduced pressure and purified by column chromatography over C18 eluting with a gradient of MeCN (0.1% NH₃) (5% to 90%; v/v) in water (0.1% NH₃) to afford the desired product N-(2-methoxybenzo[d]thiazol-5-yl)-5,6-dihydrobenzo[f]imidazo[1,5-d][1,4]oxazepine-10-carboxamide 44A (34 mg, 0.0828 mmol, 71% yield) as a white solid. ¹H NMR (400 MHz, DMSO-D6) δ 10.33 (s, 1H), 8.40 (d, J=2.3 Hz, 1H), 8.20 (d, J=2.0 Hz, 1H), 7.83 (d, J=8.6 Hz, 1H), 7.81-7.75 (m, 2H), 7.68 (d, J=1.1 Hz, 1H), 7.65 (dd, J=8.7, 2.1 Hz, 1H), 7.13 (d, J=8.5 Hz, 1H), 4.54-4.46 (m, 4H), 4.17 (s, 3H). UPLC-MS (Basic 4 min): rt=1.52 min, m z (ES+) 393.1 [M+H]⁺.

### N-(2-(2-Methoxyethoxy)benzo[d]thiazol-5-yl)-5,6-dihydrobenzo[f]imidazo[1,5-d][1,4]oxazepine-10-carboxamide 45A

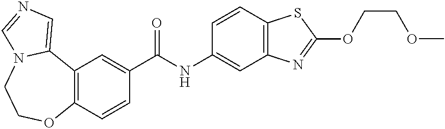 [SMILES: COCCOc1nc2cc(NC(=O)c3ccc4c(c3)-c3cncn3CCO4)ccc2s1]

To a solution of 2-methoxyethanol (0.023 mL, 0.293 mmol) in THF (2.5 mL) was added sodium hydride (60% disp. in mineral oil, 12 mg, 0.305 mmol) and the resulting suspension was stirred at room temperature for 1 h. N-(2-Chlorobenzo[d]thiazol-5-yl)-5,6-dihydrobenzo[f]imidazo[1,5-d][1,4]oxazepine-10-carboxamide 30 (50 mg, 0.117 mmol) was added and the resulting reaction mixture was stirred at room temperature for 18 h. The reaction mixture was quenched by the addition of water (2 mL) and concentrated under reduced pressure and then purified by column chromatography over C18 eluting with a gradient of MeCN(0.1% NH₃) (5% to 90%; v/v) in water (0.1% NH₃) to afford the desired product N-(2-(2-methoxyethoxy)benzo[d]thiazol-5-yl)-5,6-dihydrobenzo[f]imidazo[1,5-d][1,4]oxazepine-10-carboxamide 45A (24 mg, 0.0537 mmol, 46% yield) as an off-white solid. ¹H NMR (400 MHz, DMSO-D6) δ 10.33 (s, 1H), 8.40 (d, J=2.3 Hz, 1H), 8.19 (d, J=2.0 Hz, 1H), 7.83 (d, J=8.7 Hz, 1H), 7.80-7.77 (m, 2H), 7.68 (d, J=1.1 Hz, 1H), 7.64 (dd, J=8.7, 2.1 Hz, 1H), 7.13 (d, J=8.6 Hz, 1H), 4.70-4.63 (m, 2H), 4.53-4.46 (m, 4H), 3.76-3.71 (m, 2H), 3.32 (s, 3H). UPLC-MS (Basic 4 min): rt=1.52 min, m/z (ES+) 437.1 [M+H]⁺.

### 8-Bromo-N-((1r,4r)-4-(2-methoxyethoxy)cyclohexyl)-5,6-dihydrobenzo[f]imidazo[1,5-d][1,4]oxazepine-10-carboxamide 46A

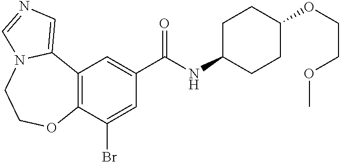 [SMILES: COCCO[C@H]1CC[C@H](NC(=O)c2cc(Br)c3c(c2)-c2cncn2CCO3)CC1]

Prepared from lithium 8-bromo-5,6-dihydrobenzo[f]imidazo[1,5-d][1,4]oxazepine-10-carboxylate and trans-4-(2-methoxyethoxy)cyclohexanamine according to General Procedure E to afford the desired product 8-bromo-N-((1r,4r)-4-(2-methoxyethoxy)cyclohexyl)-5,6-dihydrobenzo[f]imidazo[1,5-d][1,4]oxazepine-10-carboxamide (140 mg, 0.298 mmol, 57% yield) as a white solid. ¹H NMR (400 MHz, DMSO-D6) δ 8.30 (d, J=7.7 Hz, 1H), 8.21 (d, J=2.1 Hz, 1H), 7.98 (d, J=2.1 Hz, 1H), 7.83 (s, 1H), 7.61 (s, 1H), 4.57-4.46 (m, 4H), 3.81-3.69 (m, 1H), 3.57-3.50 (m, 2H), 3.45-3.38 (m, 2H), 3.30-3.22 (m, 4H), 2.05-1.98 (m, 2H), 1.90-1.82 (m, 2H), 1.44-1.30 (m, 2H), 1.30-1.16 (m, 2H). UPLC-MS (Basic 4 min): rt=1.43 min, m/z (ES+) 464.1, 466.1 [M+H]⁺.

### N-(2-(Methylamino)benzo[d]thiazol-5-yl)-5,6-dihydrobenzo[f]imidazo[1,5-d][1,4]oxazepine-10-carboxamide 47A

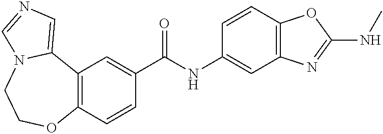 [SMILES: CNc1nc2cc(NC(=O)c3ccc4c(c3)-c3cncn3CCO4)ccc2o1]

A solution of N-(2-chlorobenzo[d]thiazol-5-yl)-5,6-dihydrobenzo[f]imidazo[1,5-d][1,4]oxazepine-10-carboxamide 30 (100 mg, 0.234 mmol), N,N-diisopropylethylamine (0.12 mL, 0.703 mmol) and methylamine (2.0 M solution in THF, 1.2 mL, 2.34 mmol) in THF (1.17 mL) was stirred at 50° C. in a sealed tube for 18 h. Additional methylamine (2.0 M solution in THF, 1.2 mL, 2.34 mmol) was added and the reaction mixture was stirred at 50° C. for 5 h. The reaction was concentrated under reduced pressure and was purified by prep HPLC to afford the desired product N-(2-(methylamino)benzo[d]thiazol-5-yl)-5,6-dihydrobenzo[f]imidazo[1,5-d][1,4]oxazepine-10-carboxamide (8.6 mg, 0.0212 mmol, 9% yield) as a white solid. ¹H NMR (400 MHz, DMSO-D6) δ 10.17 (s, 1H), 8.38 (d, J=2.2 Hz, 1H), 7.95 (q, J=4.7 Hz, 1H), 7.88 (d, J=2.0 Hz, 1H), 7.80-7.79 (m, 1H), 7.77 (dd, J=8.5, 2.2 Hz, 1H), 7.68-7.67 (m, 1H), 7.61 (d, J=8.5 Hz, 1H), 7.42 (dd, J=8.5, 2.1 Hz, 1H), 7.12 (d, J=8.6 Hz, 1H), 4.52-4.46 (m, 4H), 2.94 (d, J=4.7 Hz, 3H). UPLC-MS (Basic 4 min): rt=1.31 min, m/z (ES+) 392.1 [M+H]⁺.

### N-(2-((2-Methoxyethyl)amino)benzo[d]thiazol-5-yl)-5,6-dihydrobenzo[f]imidazo[1,5-d][1,4]oxazepine-10-carboxamide 48A

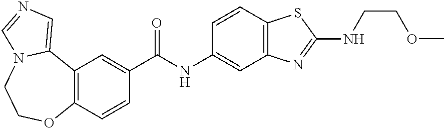 [SMILES: COCCNc1nc2cc(NC(=O)c3ccc4c(c3)-c3cncn3CCO4)ccc2s1]

A solution of N-(2-chlorobenzo[d]thiazol-5-yl)-5,6-dihydrobenzo[f]imidazo[1,5-d][1,4]oxazepine-10-carboxamide 30 (100 mg, 0.234 mmol), N,N-diisopropylethylamine (0.12 mL, 0.703 mmol) and 2-methoxyethylamine (0.203 mL, 2.34 mmol) in anhydrous DMF (1.17 mL) was stirred at 50° C. in a sealed tube for 18 h. The reaction was concentrated under reduced pressure and the residue dissolved in EtOAc (60 mL). The resulting solution was washed sequentially with water (15 mL) and brine (4×15 mL). The organic fraction was dried (Na₂SO₄) and concentrated under reduced pressure. This was purified by prep HPLC to afford the desired product N-(2-((2-methoxyethyl)amino)benzo[d]thiazol-5-yl)-5,6-dihydrobenzo[f]imidazo[1,5-d][1,4]oxazepine-10-carboxamide (46 mg, 0.105 mmol, 45% yield) as a white solid. ¹H NMR (400 MHz, DMSO-D6) δ 10.16 (s, 1H), 8.38 (d, J=2.3 Hz, 1H), 8.15-8.10 (m, 1H), 7.87 (d, J=2.0 Hz, 1H), 7.79 (d, J=1.1 Hz, 1H), 7.77 (dd, J=8.6, 2.2 Hz, 1H), 7.68 (d, J=1.1 Hz, 1H), 7.60 (d, J=8.5 Hz, 1H), 7.41 (dd, J=8.5, 2.1 Hz, 1H), 7.12 (d, J=8.6 Hz, 1H), 4.52-4.45 (m, 4H), 3.55-3.52 (m, 4H), 3.29 (s, 3H). UPLC-MS (Basic 4 min): rt=1.35 min, m/z (ES+) 436.1 [M+H]⁺.

### N-(2-(Dimethylamino)benzo[d]thiazol-5-yl)-5,6-dihydrobenzo[f]imidazo[1,5-d][1,4]oxazepine-10-carboxamide 49A

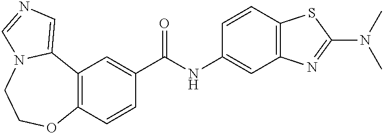 [SMILES: CN(C)c1nc2cc(NC(=O)c3ccc4c(c3)-c3cncn3CCO4)ccc2s1]

Also isolated from the previous reaction was N-(2-(dimethylamino)benzo[d]thiazol-5-yl)-5,6-dihydrobenzo[f]imidazo[1,5-d][1,4]oxazepine-10-carboxamide (31 mg, 0.0758 mmol, 32% yield) as an off-white solid. ¹H NMR (400 MHz, DMSO-D6) δ 10.19 (s, 1H), 8.38 (d, J=2.2 Hz, 1H), 7.94 (d, J=2.0 Hz, 1H), 7.80 (d, J=1.1 Hz, 1H), 7.77 (dd, J=8.6, 2.2 Hz, 1H), 7.70-7.67 (m, 2H), 7.43 (dd, J=8.5, 2.1 Hz, 1H), 7.12 (d, J=8.5 Hz, 1H), 4.52-4.45 (m, 4H), 3.15 (s, 6H). UPLC-MS (Basic 4 min): rt=1.46 min, m/z (ES+) 406.1 [M+H]⁺.

### 8-Cyclopropyl-N-((1r,4r)-4-(2-methoxyethoxy)cyclohexyl)-5,6-dihydrobenzo[f]imidazo[1,5-d][1,4]oxazepine-10-carboxamide 50A

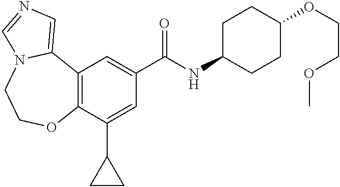 [SMILES: COCCO[C@H]1CC[C@H](NC(=O)c2cc3c(c(C4CC4)c2)OCCn2cncc2-3)CC1]

Bis (triphenylphosphine) palladium (II) dichloride (7.5 mg, 0.0106 mmol) was added to a N₂ sparged solution of 8-cyclopropyl-N-[4-(2-methoxyethoxy)cyclohexyl]-5,6-dihydroimidazo[1,5-d][1,4]benzoxazepine-10-carboxamide (50 mg, 0.106 mmol), cyclopropyl boronic acid (14 mg, 0.159 mmol) and potassium carbonate (44 mg, 0.319 mmol) in 1,4-dioxane (0.50 mL) and water (0.50 mL) and the reaction heated at 100° C. for 68 h. This was diluted with water (10 mL) and extracted with ethyl acetate (2×10 mL). The combined organic layers were dried (Na₂SO₄) and concentrated under reduced pressure. This was purified by column chromatography over C18 eluting with a gradient of MeCN(0.1% NH₃) (5% to 60%; v/v) in water (0.1% NH₃) to afford the desired product 8-cyclopropyl-N-((1r,4r)-4-(2-methoxyethoxy)cyclohexyl)-5,6-dihydrobenzo[f]imidazo[1,5-d][1,4]oxazepine-10-carboxamide (6.5 mg, 0.0146 mmol, 14% yield) as a white solid. ¹H NMR (400 MHz, DMSO-D6) δ 8.14 (d, J=7.8 Hz, 1H), 7.99 (d, J=2.2 Hz, 1H), 7.86 (s, 1H), 7.66-7.51 (m, 1H), 7.23 (d, J=2.1 Hz, 1H), 4.53-4.43 (m, 4H), 3.80-3.68 (m, 1H), 3.57-3.50 (m, 2H), 3.45-3.38 (m, 2H), 3.28-3.19 (m, 4H), 2.26-2.15 (m, 1H), 2.05-1.98 (m, 2H), 1.89-1.81 (m, 2H), 1.44-1.31 (m, 2H), 1.29-1.15 (m, 2H), 1.00-0.90 (m, 2H), 0.74-0.66 (m, 2H). UPLC-MS (Basic 4 min): rt=1.45 min, m/z (ES+) 426.5 [M+H]⁺.

### (R)-5,5-Dimethyl-N-(tetrahydro-2H-pyran-3-yl)-5,6-dihydrobenzo[f]imidazo[1,5-d][1,4]oxazepine-10-carboxamide 51A

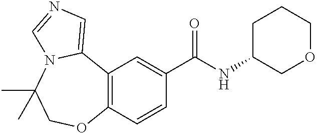 [SMILES: CC1(C)COc2ccc(C(=O)N[C@@H]3CCCOC3)cc2-c2cncn21]

Prepared from lithium 5,5-dimethyl-5,6-dihydrobenzo[f]imidazo[1,5-d][1,4]oxazepine-10-carboxylate and (R)-tetrahydro-2H-pyran-3-amine hydrochloride according to General Procedure E to afford the desired product (R)-5,5-dimethyl-N-(tetrahydro-2H-pyran-3-yl)-5,6-dihydrobenzo[f]imidazo[1,5-d][1,4]oxazepine-10-carboxamide (35 mg, 0.102 mmol, 38% yield) as a white solid. ¹H NMR (400 MHz, DMSO-D6) δ 8.27 (d, J=2.2 Hz, 1H), 8.20 (d, J=7.7 Hz, 1H), 8.04 (d, J=1.1 Hz, 1H), 7.65 (dd, J=8.5, 2.2 Hz, 1H), 7.58 (d, J=1.1 Hz, 1H), 7.07 (d, J=8.5 Hz, 1H), 4.23 (s, 2H), 3.98-3.86 (m, 1H), 3.84-3.74 (m, 2H), 3.32-3.24 (m, 1H), 3.16 (dd, J=10.7, 9.6 Hz, 1H), 1.93 (d, J=9.7 Hz, 1H), 1.76-1.54 (m, 9H). UPLC-MS (Basic 4 min): rt=1.28 min, m/z (ES+) 342.1 [M+H]⁺.

### N-((1r,4r)-4-(Trifluoromethyl)cyclohexyl)-5,6-dihydrobenzo[f]imidazo[1,5-d][1,4]oxazepine-10-carboxamide 52A

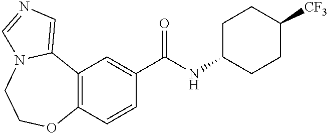 [SMILES: O=C(N[C@H]1CC[C@H](C(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and trans-4-(trifluoromethyl)cyclohexanamine hydrochloride according to General Procedure E to afford the desired product N-((1r,4r)-4-(trifluoromethyl)cyclohexyl)-5,6-dihydrobenzo[f]imidazo[1,5-d][1,4]oxazepine-10-carboxamide (110 mg, 0.287 mmol, 75% yield) as a white solid. ¹H NMR (400 MHz, DMSO-D6) δ 8.28 (d, J=7.7 Hz, 1H), 8.24 (d, J=2.2 Hz, 1H), 7.78 (d, J=1.1 Hz, 1H), 7.67 (dd, J=8.6, 2.2 Hz, 1H), 7.61 (d, J=1.1 Hz, 1H), 7.04 (d, J=8.6 Hz, 1H), 4.51-4.40 (m, 4H), 3.83-3.71 (m, 1H), 2.32-2.21 (m, 1H), 1.99-1.89 (m, 4H), 1.49-1.30 (m, 4H). ¹⁹F NMR (376 MHz, DMSO-D6) δ-71.93 (d, J=7.5 Hz). UPLC-MS (Basic 4 min): rt=1.58 min, m/z (ES+) 380.1 [M+H]⁺.

### N-((1r,4r)-4-(2-Methoxyethoxy)cyclohexyl)-8-methyl-5,6-dihydrobenzo[f]imidazo[1,5-d][1,4]oxazepine-10-carboxamide 53A

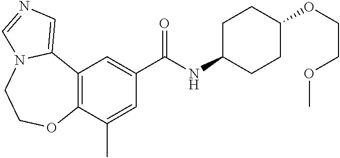 [SMILES: COCCO[C@H]1CC[C@H](NC(=O)c2cc(C)c3c(c2)-c2cncn2CCO3)CC1]

Prepared from lithium 8-methyl-5,6-dihydrobenzo[f]imidazo[1,5-d][1,4]oxazepine-10-carboxylate and trans-4-(2-methoxyethoxy)cyclohexanamine according to General Procedure E to afford the desired product N-((1r,4r)-4-(2-methoxyethoxy)cyclohexyl)-8-methyl-5,6-dihydrobenzo[f]imidazo[1,5-d][1,4]oxazepine-10-carboxamide (31 mg, 0.0773 mmol, 16% yield) as an off-white solid. ¹H NMR (400 MHz, DMSO-D6) δ 8.13 (d, J=7.8 Hz, 1H), 8.05 (d, J=2.3 Hz, 1H), 7.77 (d, J=1.1 Hz, 1H), 7.58 (dd, J=2.2, 0.9 Hz, 1H), 7.53 (d, J=1.1 Hz, 1H), 4.45 (s, 4H), 3.80-3.70 (m, 1H), 3.55-3.52 (m, 2H), 3.43-3.41 (m, 2H), 3.28-3.20 (m, 4H), 2.24 (s, 3H), 2.01 (d, J=9.7 Hz, 2H), 1.85 (d, J=12.5 Hz, 2H), 1.43-1.32 (m, 2H), 1.27-1.17 (m, 2H). UPLC-MS (Basic 4 min): rt=1.35 min, m/z (ES+) 400.2 [M+H]⁺.

### 8-Methoxy-N-((1r,4r)-4-(2-methoxyethoxy)cyclohexyl)-5,6-dihydrobenzo[f]imidazo[1,5-d][1,4]oxazepine-10-carboxamide 54A

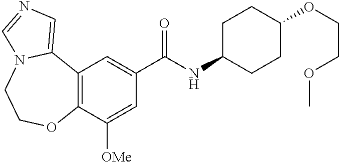 [SMILES: COCCO[C@H]1CC[C@H](NC(=O)c2cc(OC)c3c(c2)-c2cncn2CCO3)CC1]

Prepared from lithium 8-methoxy-5,6-dihydrobenzo[f]imidazo[1,5-d][1,4]oxazepine-10-carboxylate and trans-4-(2-methoxyethoxy)cyclohexanamine according to General Procedure E to afford the desired product 8-methoxy-N-((1r,4r)-4-(2-methoxyethoxy)cyclohexyl)-5,6-dihydrobenzo[f]imidazo[1,5-d][1,4]oxazepine-10-carboxamide (62 mg, 0.150 mmol, 31% yield) as a white solid. ¹H NMR (400 MHz, DMSO-D6) δ 8.17 (d, J=7.8 Hz, 1H), 7.82 (d, J=2.1 Hz, 1H), 7.78 (d, J=1.1 Hz, 1H), 7.56 (d, J=1.1 Hz, 1H), 7.32 (d, J=2.0 Hz, 1H), 4.47-4.41 (m, 4H), 3.82 (s, 3H), 3.81-3.71 (m, 1H), 3.56-3.52 (m, 2H), 3.44-3.41 (m, 2H), 3.31-3.18 (m, 4H), 2.04-1.97 (m, 2H), 1.87 (d, J=12.2 Hz, 2H), 1.45-1.33 (m, 2H), 1.28-1.17 (m, 2H). UPLC-MS (Basic 4 min): rt=1.19 min, m/z (ES+) 416.2 [M+H]⁺.

### N-((1r,4r)-4-(2-Methoxyethoxy)cyclohexyl)-8-(trifluoromethyl)-5,6-dihydrobenzo[f]imidazo[1,5-d][1,4]oxazepine-10-carboxamide 55A

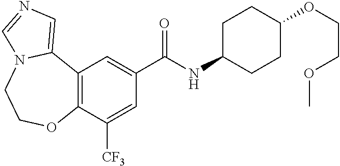 [SMILES: COCCO[C@H]1CC[C@H](NC(=O)c2cc3c(c(C(F)(F)F)c2)OCCn2cncc2-3)CC1]

Prepared from lithium 8-(trifluoromethyl)-5,6-dihydrobenzo[f]imidazo[1,5-d][1,4]oxazepine-10-carboxylate and trans-4-(2-methoxyethoxy)cyclohexanamine according to General Procedure E to afford the desired product N-((1r,4r)-4-(2-methoxyethoxy)cyclohexyl)-8-(trifluoromethyl)-5,6-dihydrobenzo[f]imidazo[1,5-d][1,4]oxazepine-10-carboxamide (30 mg, 0.0665 mmol, 28% yield) as a white solid. ¹H NMR (400 MHz, DMSO-D6) δ 8.49 (d, J=2.2 Hz, 1H), 8.43 (d, J=7.7 Hz, 1H), 8.00 (d, J=2.1 Hz, 1H), 7.87 (d, J=1.1 Hz, 1H), 7.68 (d, J=1.1 Hz, 1H), 4.60-4.50 (m, 4H), 3.84-3.70 (m, 1H), 3.57-3.50 (m, 2H), 3.46-3.37 (m, 2H), 3.29-3.21 (m, 4H), 2.07-1.99 (m, 2H), 1.92-1.84 (m, 2H), 1.46-1.31 (m, 2H), 1.31-1.16 (m, 2H). ¹⁹F NMR (376 MHz, DMSO-D6) δ-60.68 (s). UPLC-MS (Basic 4 min): rt=1.50 min, m/z (ES+) 454.2 [M+H]⁺.

### N-((1r,4r)-4-(2-Hydroxyethyl)cyclohexyl)-5,6-dihydrobenzo[f]imidazo[1,5-d][1,4]oxazepine-10-carboxamide 56A

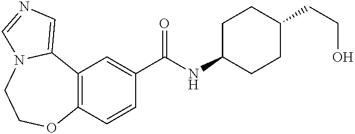 [SMILES: O=C(N[C@H]1CC[C@H](CCO)CC1)c1ccc2c(c1)-c1cncn1CCO2]

To a suspension of lithium 5,6-dihydrobenzo[f]imidazo[1,5-d][1,4]oxazepine-10-carboxylate 4 (100 mg, 0.423 mmol), O-(7-azabenzotriazol-1-yl)-N,N,N′,N′-tetramethyluronium hexafluorophosphate (HATU) (242 mg, 0.635 mmol) and trans-2-(4-aminocyclohexyl) ethanol hydrochloride (152 mg, 0.847 mmol) in DMF (4 mL) was added N,N-diisopropylethylamine (0.22 mL, 1.27 mmol). The resulted reaction mixture was allowed to stir at 25° C. for 60 h. This was diluted with water (10 mL), the precipitated solid was filtered and dried under vacuum to afford the desired product N-((1r,4r)-4-(2-hydroxyethyl)cyclohexyl)-5,6-dihydrobenzo[f]imidazo[1,5-d][1,4]oxazepine-10-carboxamide (92 mg, 0.256 mmol, 61% yield) as an off-white solid. ¹H NMR (400 MHz, DMSO-D6) δ 8.24 (d, J=2.2 Hz, 1H), 8.19 (d, J=7.9 Hz, 1H), 7.77 (d, J=1.1 Hz, 1H), 7.70-7.58 (m, 2H), 7.04 (d, J=8.6 Hz, 1H), 4.51-4.39 (m, 4H), 4.34 (t, J=5.1 Hz, 1H), 3.77-3.71 (m, 1H), 3.47-3.42 (m, 2H), 1.98-1.79 (m, 2H), 1.77 (d, J=12.9 Hz, 2H), 1.39-1.29 (m, 5H), 1.04-0.95 (m, 2H). UPLC-MS (Basic 4 min): rt=1.16 min, m/z (ES+) 356.4 [M+H]⁺.

### 8-Fluoro-N-((1r,4r)-4-(2-methoxyethoxy)cyclohexyl)-5,6-dihydrobenzo[f]imidazo[1,5-d][1,4]oxazepine-10-carboxamide 57A

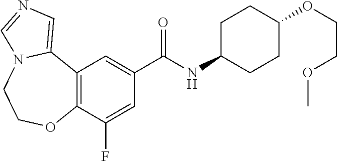 [SMILES: COCCO[C@H]1CC[C@H](NC(=O)c2cc(F)c3c(c2)-c2cncn2CCO3)CC1]

Prepared from lithium 8-fluoro-5,6-dihydrobenzo[f]imidazo[1,5-d][1,4]oxazepine-10-carboxylate and trans-4-(2-methoxyethoxy)cyclohexanamine according to General Procedure E to afford the desired product 8-fluoro-N-((1r,4r)-4-(2-methoxyethoxy)cyclohexyl)-5,6-dihydrobenzo[f]imidazo[1,5-d][1,4]oxazepine-10-carboxamide (39 mg, 0.0966 mmol, 26% yield) as a white solid. ¹H NMR (400 MHz, DMSO-D6) δ 8.25 (d, J=7.7 Hz, 1H), 8.06 (t, J=1.8 Hz, 1H), 7.81 (d, J=1.1 Hz, 1H), 7.64 (d, J=1.2 Hz, 1H), 7.57 (dd, J=11.5, 2.1 Hz, 1H), 4.53 (s, 4H), 3.82-3.68 (m, 1H), 3.57-3.50 (m, 2H), 3.45-3.38 (m, 2H), 3.28-3.19 (m, 4H), 2.06-1.97 (m, 2H), 1.91-1.82 (m, 2H), 1.45-1.30 (m, 2H), 1.30-1.15 (m, 2H).

¹⁹F NMR (376 MHz, DMSO-D6) δ-130.87 (s, F). UPLC-MS (Basic 4 min): rt=1.29 min, m/z (ES+) 404.3 [M+H]⁺.

### N-((1r,4r)-4-(2-Hydroxypropan-2-yl)cyclohexyl)-5,6-dihydrobenzo[f]imidazo[1,5-d][1,4]oxazepine-10-carboxamide 58A

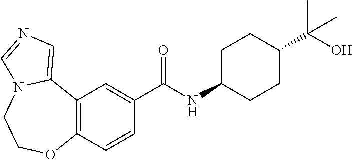 [SMILES: CC(C)(O)[C@H]1CC[C@H](NC(=O)c2ccc3c(c2)-c2cncn2CCO3)CC1]

To a suspension of lithium 5,6-dihydrobenzo[f]imidazo[1,5-d][1,4]oxazepine-10-carboxylate 4 (80 mg, 0.339 mmol), O-(7-azabenzotriazol-1-yl)-N,N,N′,N′-tetramethyluronium hexafluorophosphate (HATU) (193 mg, 0.508 mmol) and trans-2-(4-aminocyclohexyl) propan-2-ol (128 mg, 0.423 mmol) in DMF (4 mL) was added N,N-diisopropylethylamine (0.177 mL, 1.02 mmol). The resulted reaction mixture was allowed to stir at 25° C. for 18 h. This was concentrated under reduced pressure and purified by column chromatography over C18 eluting with a gradient of MeCN(0.1% NH₃) (5% to 95%; v/v) in water (0.1% NH₃), followed by prep HPLC purification to afford the desired product N-((1r,4r)-4-(2-hydroxypropan-2-yl)cyclohexyl)-5,6-dihydrobenzo[f]imidazo[1,5-d][1,4]oxazepine-10-carboxamide (70 mg, 0.189 mmol, 56% yield) as a white solid. ¹H NMR (400 MHz, DMSO-D6) δ 8.24 (d, J=2.2 Hz, 1H), 8.17 (d, J=7.9 Hz, 1H), 7.77 (d, J=1.1 Hz, 1H), 7.66 (dd, J=8.6, 2.2 Hz, 1H), 7.62 (d, J=1.1 Hz, 1H), 7.03 (d, J=8.5 Hz, 1H), 4.51-4.39 (m, 4H), 4.05 (s, 1H), 3.77-3.64 (m, 1H), 1.93-1.80 (m, 4H), 1.38-1.25 (m, 2H), 1.20-1.02 (m, 9H). UPLC-MS (Basic 4 min): rt=1.25 min, m/z (ES+) 370.2 [M+H]⁺.

### N-((1r,4r)-4-((2-Methoxyethyl)carbamoyl)cyclohexyl)-5,6-dihydrobenzo[f]imidazo[1,5-d][1,4]oxazepine-10-carboxamide 59A

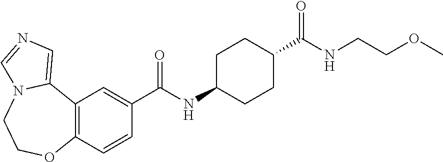 [SMILES: COCCNC(=O)[C@H]1CC[C@H](NC(=O)c2ccc3c(c2)-c2cncn2CCO3)CC1]

Prepared from lithium (1r,4r)-4-(5,6-dihydrobenzo[f]imidazo[1,5-d][1,4]oxazepine-10-carboxamido)cyclohexane-1-carboxylate 32 and according to General Procedure E to afford the desired product N-((1r,4r)-4-((2-methoxyethyl)carbamoyl)cyclohexyl)-5,6-dihydrobenzo[f]imidazo[1,5-d][1,4]oxazepine-10-carboxamide (69 mg, 0.160 mmol, 63% yield) as a white solid. ¹H NMR (400 MHz, DMSO-D6) δ 8.24 (d, J=2.2 Hz, 1H), 8.20 (d, J=8.0 Hz, 1H), 7.82 (t, J=5.7 Hz, 1H), 7.77 (d, J=1.1 Hz, 1H), 7.66 (dd, J=8.6, 2.2 Hz, 1H), 7.61 (d, J=1.1 Hz, 1H), 7.04 (d, J=8.5 Hz, 1H), 4.51-4.39 (m, 4H), 3.86-3.67 (m, 1H), 3.24 (s, 3H), 3.19 (dt, J=5.8, 5.7 Hz, 2H), 2.16-2.05 (m, 1H), 1.92-1.84 (m, 2H), 1.80-1.74 (m, 2H), 1.52-1.28 (m, 4H). 2H not observed. UPLC-MS (Basic 4 min): rt=1.09 min, m/z (ES+) 413.3 [M+H]⁺.

### N-((1r,4r)-4-Acetamidocyclohexyl)-5,6-dihydrobenzo[f]imidazo[1,5-d][1,4]oxazepine-10-carboxamide 60A

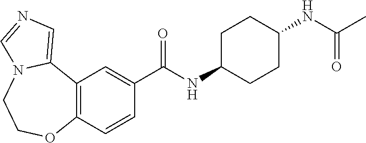 [SMILES: CC(=O)N[C@H]1CC[C@H](NC(=O)c2ccc3c(c2)-c2cncn2CCO3)CC1]

Acetyl chloride (0.030 mL, 0.413 mmol) was added to a suspension of N-((1r,4r)-4-aminocyclohexyl)-5,6-dihydrobenzo[f]imidazo[1,5-d][1,4]oxazepine-10-carboxamide hydrochloride 23 (100 mg, 0.276 mmol) and N,N-diisopropylethylamine (0.14 mL, 0.827 mmol) in DCM (2 mL) at 25° C. and stirred for 3 h. This was concentrated under reduced pressure and then triturated with water: MeCN(4:1, 20 mL). The resulting solid was dried under reduced pressure. The crude material was purified by column chromatography over C18 eluting with a gradient of MeCN(0.1% NH₃) (5% to 95%: v/v) in water (0.1% NH₃) and the resulting solid triturated with DMSO to afford the desired product N-((1r,4r)-4-acetamidocyclohexyl)-5,6-dihydrobenzo[f]imidazo[1,5-d][1,4]oxazepine-10-carboxamide (9.0 mg, 0.0230 mmol, 8% yield) as an off-white solid. ¹H NMR (400 MHz, DMSO-D6) δ 8.26-8.18 (m, 2H), 7.79-7.72 (m, 2H), 7.66 (dd, J=8.6, 2.2 Hz, 1H), 7.61 (d, J=1.1 Hz, 1H), 7.04 (d, J=8.5 Hz, 1H), 4.51-4.39 (m, 4H), 3.81-3.69 (m, 1H), 3.54-3.45 (m, 1H), 1.90-1.80 (m, 4H), 1.78 (s, 3H), 1.48-1.34 (m, 2H), 1.31-1.17 (m, 2H). UPLC-MS (Basic 4 min): rt=1.03 min, m/z (ES+) 369.2 [M+H]⁺.

### N-((1r,4r)-4-((2,2,2-Trifluoroethyl)carbamoyl)cyclohexyl)-5,6-dihydrobenzo[f]imidazo[1,5-d][1,4]oxazepine-10-carboxamide 61A

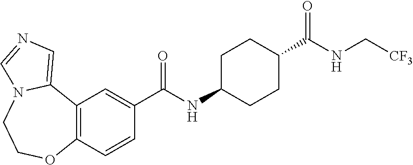 [SMILES: O=C(N[C@H]1CC[C@H](C(=O)NCC(F)(F)F)CC1)c1ccc2c(c1)-c1cncn1CCO2]

Prepared from lithium (1r,4r)-4-(5,6-dihydrobenzo[f]imidazo[1,5-d][1,4]oxazepine-10-carboxamido)cyclohexane-1-carboxylate 31 and 2,2,2-trifluoroethylamine according to General Procedure E to afford the desired product N-((1r,4r)-4-((2,2,2-trifluoroethyl)carbamoyl)cyclohexyl)-5,6-dihydrobenzo[f]imidazo[1,5-d][1,4]oxazepine-10-carboxamide (83 mg, 0.187 mmol, 73% yield) as a white solid. ¹H NMR (400 MHz, DMSO-D6) δ 8.44 (t, J=6.4 Hz, 1H), 8.24 (d, J=2.2 Hz, 1H), 8.20 (d, J=7.9 Hz, 1H), 7.77 (d, J=1.1 Hz, 1H), 7.66 (dd, J=8.6, 2.2 Hz, 1H), 7.61 (d, J=1.1 Hz, 1H), 7.04 (d, J=8.6 Hz, 1H), 4.51-4.40 (m, 4H), 3.95-3.82 (m, 2H), 3.82-3.68 (m, 1H), 2.26-2.15 (m, 1H), 1.94-1.87 (m, 2H), 1.84-1.77 (m, 2H), 1.54-1.30 (m, 4H). ¹⁹F NMR (376 MHz, DMSO-D6) δ-70.78 (t, J=9.8 Hz, CF₃). UPLC-MS (Basic 4 min): rt=1.26 min, m/z (ES+) 437.2 [M+H]⁺.

### N-((1r,4r)-4-((2,2,2-Trifluoroethyl)amino)cyclohexyl)-5,6-dihydrobenzo[f]imidazo[1,5-d][1,4]oxazepine-10-carboxamide 62A

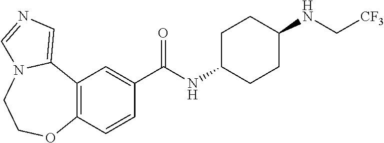 [SMILES: O=C(N[C@H]1CC[C@H](NCC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and trans-N4-(2,2,2-trifluoroethyl)cyclohexane-1,4-diamine according to General Procedure E to afford the desired product N-((1r,4r)-4-((2,2,2-trifluoroethyl)amino)cyclohexyl)-5,6-dihydrobenzo[f]imidazo[1,5-d][1,4]oxazepine-10-carboxamide (37 mg, 0.0891 mmol, 30% yield) as a white solid. ¹H NMR (400 MHz, DMSO-D6) δ 8.23 (d, J=2.2 Hz, 1H), 8.17 (d, J=7.9 Hz, 1H), 7.77 (d, J=1.1 Hz, 1H), 7.65 (dd, J=8.6, 2.2 Hz, 1H), 7.61 (d, J=1.1 Hz, 1H), 7.03 (d, J=8.5 Hz, 1H), 4.51-4.39 (m, 4H), 3.80-3.67 (m, 1H), 3.30-3.19 (m, 2H), 2.47-2.35 (m, 1H), 2.21 (dt, J=7.4, 7.4 Hz, 1H), 1.98-1.89 (m, 2H), 1.88-1.79 (m, 2H), 1.42-1.28 (m, 2H), 1.17-1.04 (m, 2H). ¹⁹F NMR (376 MHz, DMSO-D6) δ-70.57 (t, J=10.3 Hz). UPLC-MS (Basic 6 min): rt=2.20 min, m/z (ES+) 409.2 [M+H]⁺.

### N-((1r,4r)-4-((2,2-Difluoroethyl)amino)cyclohexyl)-5,6-dihydrobenzo[f]imidazo[1,5-d][1,4]oxazepine-10-carboxamide 63A

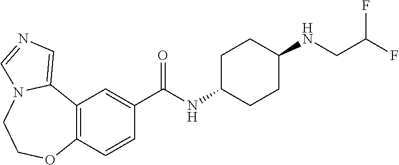 [SMILES: O=C(N[C@H]1CC[C@H](NCC(F)F)CC1)c1ccc2c(c1)-c1cncn1CCO2]

Prepared from lithium 5,6-dihydrobenzo[f]imidazo[1,5-d][1,4]oxazepine-10-carboxylate 4 and trans-N4-(2,2-difluoroethyl)cyclohexane-1,4-diamine according to General Procedure E to afford the desired product N-((1r,4r)-4-((2,2-difluoroethyl)amino)cyclohexyl)-5,6-dihydrobenzo[f]imidazo[1,5-d][1,4]oxazepine-10-carboxamide (23 mg, 0.0582 mmol, 28% yield) as a white solid. ¹H NMR (400 MHz, DMSO-D6) δ 8.23 (d, J=2.2 Hz, 1H), 8.18 (d, J=7.9 Hz, 1H), 7.77 (d, J=1.1 Hz, 1H), 7.66 (dd, J=8.6, 2.2 Hz, 1H), 7.61 (d, J=1.1 Hz, 1H), 7.03 (d, J=8.6 Hz, 1H), 5.95 (tt, J=56.5, 4.4 Hz, 1H), 4.51-4.39 (m, 4H), 3.80-3.65 (m, 1H), 2.92 (td, J=15.9, 4.4 Hz, 2H), 2.46-2.33 (m, 1H), 2.01-1.89 (m, 2H), 1.87-1.76 (m, 2H), 1.43-1.29 (m, 2H), 1.17-1.03 (m, 2H)-1H not observed. ¹⁹F NMR (376 MHz, DMSO-D6) 8-120.31 (dt, J=56.7, 15.9 Hz). UPLC-MS (Basic 6 min): rt=1.94 min, m/z (ES+) 391.2 [M+H]⁺.

### N-((1s,4s)-4-(3,3-Difluoroazetidin-1-yl)cyclohexyl)-5,6-dihydrobenzo[f]imidazo[1,5-d][1,4]oxazepine-10-carboxamide 64A

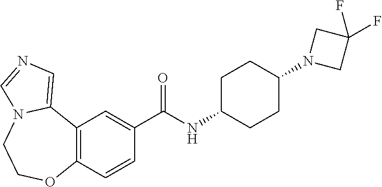 [SMILES: O=C(N[C@H]1CC[C@@H](N2CC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and cis-4-(3,3-difluoroazetidin-1-yl)cyclohexanamine according to General Procedure E to afford the desired product N-((1s,4s)-4-(3,3-difluoroazetidin-1-yl)cyclohexyl)-5,6-dihydrobenzo[f]imidazo[1,5-d][1,4]oxazepine-10-carboxamide (9.0 mg, 0.0215 mmol, 20% yield) as a white solid. ¹H NMR (400 MHz, DMSO-D6) δ 8.31-8.17 (m, 2H), 7.76 (s, 1H), 7.72-7.58 (m, 2H), 7.02 (d, J=8.6 Hz, 1H), 4.51-4.39 (m, 4H), 3.90-3.77 (m, 1H), 3.60-3.47 (m, 4H), 2.42-2.38 (m, 1H), 1.78-1.58 (m, 3H), 1.54-1.39 (m, 4H). 1H not observed. 19F NMR (376 MHz, DMSO-D6) δ-98.12 (s). UPLC-MS (Basic 6 min): rt=2.26 min, m/z (ES+) 403.2 [M+H]⁺.

### N-((1r,4r)-4-(Hydroxymethyl)cyclohexyl)-5,6-dihydrobenzo[f]imidazo[1,5-d][1,4]oxazepine-10-carboxamide 65A

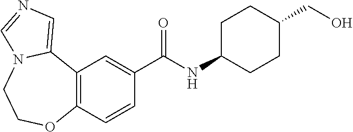 [SMILES: O=C(N[C@H]1CC[C@H](CO)CC1)c1ccc2c(c1)-c1cncn1CCO2]

To a suspension of lithium 5,6-dihydrobenzo[f]imidazo[1,5-d][1,4]oxazepine-10-carboxylate 4 (100 mg, 0.424 mmol), O-(7-azabenzotriazol-1-yl)-N,N,N′,N′-tetramethyluronium hexafluorophosphate (241 mg, 0.635 mmol) and trans-(4-aminocyclohexyl) methanol (84.4 mg, 0.529 mmol) in DMF (4 mL) was added N,N-diisopropylethylamine (0.221 mL, 1.27 mmol). The resulted reaction mixture was allowed to stir at 25° C. for 18 h. This was concentrated under reduced pressure and purified by column chromatography over C18 eluting with a gradient of MeCN(0.1% NH₃) (5% to 95%; v/v) in water (0.1% NH₃), followed by prep HPLC purification to afford the desired product N-((1r,4r)-4-(hydroxymethyl)cyclohexyl)-5,6-dihydrobenzo[f]imidazo[1,5-d][1,4]oxazepine-10-carboxamide (74 mg, 0.212 mmol, 50% yield) as a yellow solid. ¹H NMR (400 MHz, DMSO-D6) δ 8.24 (d, J=2.2 Hz, 1H), 8.19 (d, J=8.0 Hz, 1H), 7.77 (d, J=1.1 Hz, 1H), 7.66 (dd, J=8.6, 2.2 Hz, 1H), 7.61 (d, J=1.1 Hz, 1H), 7.03 (d, J=8.5 Hz, 1H), 4.51-4.46 (m, 2H), 4.43 (dt, J=6.0, 1.8 Hz, 3H), 3.88-3.59 (m, 1H), 3.23 (d, J=6.3 Hz, 2H), 1.93-1.73 (m, 4H), 1.58-1.16 (m, 3H), 1.12-0.82 (m, 2H). UPLC-MS (Basic 4 min): rt=1.08 min, m/z (ES+) 342.3 [M+H]⁺.

### N-(3,3-Difluorocyclopentyl)-5,6-dihydrobenzo[f]imidazo[1,5-d][1,4]oxazepine-10-carboxamide 66A

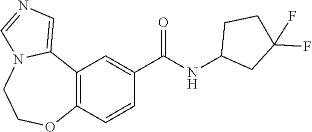 [SMILES: O=C(NC1CCC(F)(F)C1)c1ccc2c(c1)-c1cncn1CCO2]

Prepared from lithium 5,6-dihydrobenzo[f]imidazo[1,5-d][1,4]oxazepine-10-carboxylate 4 and 3,3-difluorocyclopentan-1-amine hydrochloride according to General Procedure E to afford the desired product N-(3,3-difluorocyclopentyl)-5,6-dihydrobenzo[f]imidazo[1,5-d][1,4]oxazepine-10-carboxamide (86 mg, 0.255 mmol, 60% yield) as an off-white solid. ¹H NMR (400 MHz, DMSO-D6) δ 8.48 (d, J=7.1 Hz, 1H), 8.21 (d, J=2.2 Hz, 1H), 7.74 (d, J=1.1 Hz, 1H), 7.63 (dd, J=8.6, 2.2 Hz, 1H), 7.57 (d, J=1.1 Hz, 1H), 7.02 (d, J=8.6 Hz, 1H), 4.48-4.32 (m, 5H), 2.29-1.96 (m, 4H), 1.81-1.77 (m, 1H). 1H not observed. ¹⁹F NMR (376 MHz, DMSO-D6) δ-84.69 (s), −87.76 (s). UPLC-MS (Basic 4 min): rt=1.30 min, m/z (ES+) 334.3 [M+H]⁺.

### N-((1r,4r)-4-(3-Methoxypropanamido)cyclohexyl)-5,6-dihydrobenzo[f]imidazo[1,5-d][1,4]oxazepine-10-carboxamide 67A

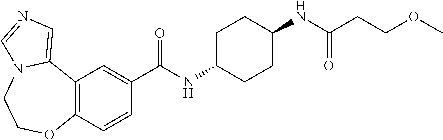 [SMILES: COCCC(=O)N[C@H]1CC[C@H](NC(=O)c2ccc3c(c2)-c2cncn2CCO3)CC1]

Prepared from N-((1r,4r)-4-aminocyclohexyl)-5,6-dihydrobenzo[f]imidazo[1,5-d][1,4]oxazepine-10-carboxamide hydrochloride 23 and 3-methoxypropanoic acid according to General Procedure E to afford the desired product N-((1r,4r)-4-(3-methoxypropanamido)cyclohexyl)-5,6-dihydrobenzo[f]imidazo[1,5-d][1,4]oxazepine-10-carboxamide (24 mg, 0.0581 mmol, 26% yield) as a white solid. ¹H NMR (400 MHz, DMSO-D6) δ 8.24 (d, J=2.3 Hz, 1H), 8.21 (d, J=7.9 Hz, 1H), 7.80-7.75 (m, 2H), 7.66 (dd, J=8.6, 2.2 Hz, 1H), 7.61 (d, J=1.2 Hz, 1H), 7.04 (d, J=8.5 Hz, 1H), 4.49-4.41 (m, 4H), 3.80-3.69 (m, 1H), 3.55-3.47 (m, 3H), 3.20 (s, 3H), 2.28 (t, J=6.4 Hz, 2H), 1.89-1.80 (m, 4H), 1.47-1.35 (m, 2H), 1.30-1.20 (m, 2H). UPLC-MS (Basic 4 min): rt=1.08 min, m/z (ES+) 413.3 [M+H]⁺.

### 5-Methyl-N—((R)-tetrahydro-2H-pyran-3-yl)-5,6-dihydrobenzo[f]imidazo[1,5-d][1,4]oxazepine-10-carboxamide 68A

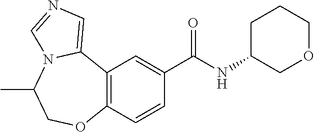 [SMILES: CC1COc2ccc(C(=O)N[C@@H]3CCCOC3)cc2-c2cncn21]

Prepared from lithium 5,5-dimethyl-5,6-dihydrobenzo[f]imidazo[1,5-d][1,4]oxazepine-10-carboxylate and (R)-tetrahydro-2H-pyran-3-amine hydrochloride according to General Procedure E to afford the desired product 5-methyl-N—((R)-tetrahydro-2H-pyran-3-yl)-5,6-dihydrobenzo[f]imidazo[1,5-d][1,4]oxazepine-10-carboxamide (42 mg, 0.127 mmol, 34% yield) as a white solid. ¹H NMR (400 MHz, DMSO-D6) δ 8.22 (d, J=2.2 Hz, 1H), 8.15 (d, J=7.8 Hz, 1H), 7.82 (d, J=1.1 Hz, 1H), 7.62 (dd, J=8.6, 2.2 Hz, 1H), 7.56 (d, J=1.1 Hz, 1H), 7.03 (d, J=8.5 Hz, 1H), 4.83-4.75 (m, 1H), 4.44 (dd, J=12.9, 4.6 Hz, 1H), 4.24 (dd, J=12.9, 1.4 Hz, 1H), 3.93-3.82 (m, 1H), 3.80-3.71 (m, 2H), 3.28-3.21 (m, 1H), 3.16-3.08 (m, 1H), 1.94-1.83 (m, 1H), 1.70-1.62 (m, 1H), 1.61-1.48 (m, 2H), 1.40 (d, J=6.8 Hz, 3H). UPLC-MS (Basic 4 min): rt=1.18 min, m/z (ES+) 328.1 [M+H]⁺.

### N-((1s,4s)-4-(3,3-Difluoropyrrolidin-1-yl)cyclohexyl)-5,6-dihydrobenzo[f]imidazo[1,5-d][1,4]oxazepine-10-carboxamide 69A

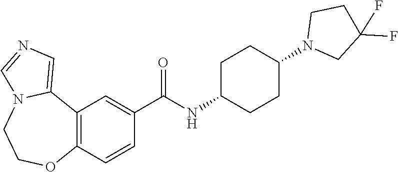 [SMILES: O=C(N[C@H]1CC[C@@H](N2CCC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and cis-4-(3,3-difluoropyrrolidin-1-yl)cyclohexanamine according to General Procedure E to afford the desired product N-((1s,4s)-4-(3,3-difluoropyrrolidin-1-yl)cyclohexyl)-5,6-dihydrobenzo[f]imidazo[1,5-d][1,4]oxazepine-10-carboxamide (7.5 mg, 0.0176 mmol, 17% yield) as a white solid. ¹H NMR (400 MHz, DMSO-D6) δ 8.30-8.24 (m, 2H), 7.76 (d, J=1.1 Hz, 1H), 7.69 (dd, J=8.6, 2.2 Hz, 1H), 7.63 (d, J=1.1 Hz, 1H), 7.02 (d, J=8.6 Hz, 1H), 4.50-4.39 (m, 4H), 3.93-3.80 (m, 1H), 2.92 (t, J=13.9 Hz, 2H), 2.72 (t, J=7.0 Hz, 2H), 1.87-1.79 (m, 2H), 1.77-1.66 (m, 2H), 1.59-1.44 (m, 4H). 1H not observed. ¹⁹F NMR (376 MHz, DMSO-D6) δ-90.05 (q, J=14.6 Hz). UPLC-MS (Basic 4 min): rt=1.52 min, m/z (ES+) 417.3 [M+H]⁺.

### N-((1r,4r)-4-(2-Methoxyethoxy)cyclohexyl)-6,7-dihydroimidazo[1,5-d]pyrido[3,2-b][1,4]oxazepine-2-carboxamide 70A

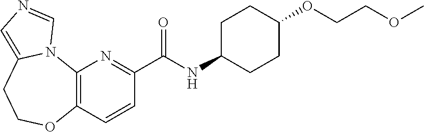 [SMILES: COCCO[C@H]1CC[C@H](NC(=O)c2ccc3c(n2)-n2cncc2CCO3)CC1]

A suspension of 5-(2-(1H-imidazol-5-yl)ethoxy)-6-bromo-N-((1r,4r)-4-(2-methoxyethoxy)cyclohexyl) picolinamide (20 mg, 0.0428 mmol) and cesium carbonate (41.8 mg, 0.128 mmol) in NMP (1 mL) was heated to 100° C. for 1.5 h. The reaction mixture was dissolved in DCM (20 mL) and washed with water (15 mL). The organic fraction was dried (Na₂SO₄) and concentrated under reduced pressure. This was purified by prep HPLC to afford the desired product N-((1r,4r)-4-(2-methoxyethoxy)cyclohexyl)-6,7-dihydroimidazo[1,5-d]pyrido[3,2-b][1,4]oxazepine-2-carboxamide (1.8 mg, 0.0047 mmol, 11% yield) as a beige solid. ¹H NMR (400 MHz, ACETONE-D6) δ 8.54 (d, J=1.1 Hz, 1H), 8.09-8.04 (m, 1H), 8.02 (d, J=8.3 Hz, 1H), 7.71 (d, J=8.3 Hz, 1H), 6.89 (d, J=1.1 Hz, 1H), 4.52 (dd, J=5.8, 4.9 Hz, 2H), 4.03-3.81 (m, 1H), 3.58 (dd, J=5.9, 4.1 Hz, 2H), 3.46 (dd, J=5.9, 4.1 Hz, 2H), 3.36-3.20 (m, 1H), 3.30 (s, 3H), 3.29-3.25 (m, 2H), 2.11-1.93 (m, 4H), 1.66-1.48 (m, 2H), 1.43-1.20 (m, 2H). UPLC-MS (Basic 4 min): rt=1.29 min, m/z (ES+) 387.4 [M+H]⁺.

### N-((1r,4r)-4-(3,3-Difluoropyrrolidin-1-yl)cyclohexyl)-5,6-dihydrobenzo[f]imidazo[1,5-d][1,4]oxazepine-10-carboxamide 71A

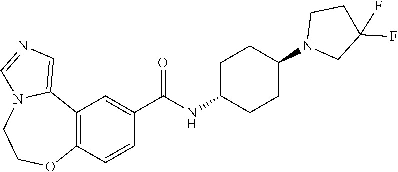 [SMILES: O=C(N[C@H]1CC[C@H](N2CCC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and trans-4-(3,3-difluoropyrrolidin-1-yl)cyclohexanamine according to General Procedure E to afford the desired product N-((1r,4r)-4-(3,3-difluoropyrrolidin-1-yl)cyclohexyl)-5,6-dihydrobenzo[f]imidazo[1,5-d][1,4]oxazepine-10-carboxamide (50.4 mg, 0.121 mmol, 29% yield) as a white solid. ¹H NMR (400 MHz, DMSO-D6) δ 8.27-8.19 (m, 2H), 7.89 (s, 1H), 7.70-7.64 (m, 2H), 7.04 (d, J=8.6 Hz, 1H), 4.52-4.48 (m, 2H), 4.46-4.42 (m, 2H), 3.80-3.68 (m, 1H), 3.12-2.96 (m, 2H), 2.84 (s, 2H), 2.32-2.16 (m, 3H), 1.99-1.94 (m, 2H), 1.88 (d, J=12.1 Hz, 2H), 1.42-1.36 (m, 2H), 1.29-1.20 (m, 2H). ¹⁹F NMR (376 MHz, DMSO-D6) 8-90.80 (app s). UPLC-MS (Basic 4 min): rt=1.38 min, m/z (ES+) 417.4 [M+H]⁺.

### N-((1r,4r)-4-(3,3-Difluoroazetidin-1-yl)cyclohexyl)-5,6-dihydrobenzo[f]imidazo[1,5-d][1,4]oxazepine-10-carboxamide 72A

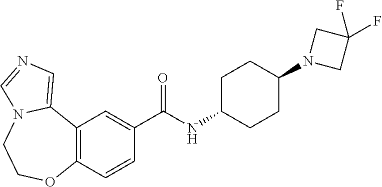 [SMILES: O=C(N[C@H]1CC[C@H](N2CC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and trans-4-(3,3-difluoroazetidin-1-yl)cyclohexanamine according to General Procedure E to afford the desired product N-((1r,4r)-4-(3,3-difluoroazetidin-1-yl)cyclohexyl)-5,6-dihydrobenzo[f]imidazo[1,5-d][1,4]oxazepine-10-carboxamide (64 mg, 0.157 mmol, 27% yield) as an off-white solid. ¹H NMR (400 MHz, DMSO-D6) δ 8.22-8.13 (m, 2H), 7.73 (d, J=1.1 Hz, 1H), 7.62 (dd, J=8.5, 2.2 Hz, 1H), 7.57 (d, J=1.1 Hz, 1H), 7.00 (d, J=8.6 Hz, 1H), 4.47-4.41 (m, 2H), 4.42-4.36 (m, 2H), 3.69-3.66 (m, 1H), 3.60-3.45 (m, 4H), 2.15-2.00 (m, J=17.4 Hz, 1H), 1.85-1.70 (m, 4H), 1.37-1.31 (m, 2H), 1.09-0.95 (m, 2H). ¹⁹F NMR (376 MHz, DMSO-D6) δ-98.11 (m). UPLC-MS (Basic 4 min): rt=1.29 min, m/z (ES+) 403.4 [M+H]⁺.

### N-(4,4-Difluorocyclohexyl)-8-fluoro-5,6-dihydrobenzo[f]imidazo[1,5-d][1,4]oxazepine-10-carboxamide 73A

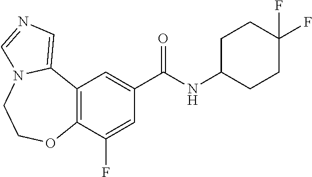 [SMILES: O=C(NC1CCC(F)(F)CC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 4,4-difluorocyclohexanamine according to General Procedure E to afford the desired product N-(4,4-difluorocyclohexyl)-8-fluoro-5,6-dihydrobenzo[f]imidazo[1,5-d][1,4]oxazepine-10-carboxamide (94 mg, 0.255 mmol, 49% yield) as a pink solid. ¹H NMR (400 MHz, DMSO-D6) δ 8.29 (d, J=7.7 Hz, 1H), 8.04 (t, J=1.8 Hz, 1H), 7.78 (d, J=1.1 Hz, 1H), 7.61 (d, J=1.1 Hz, 1H), 7.54 (dd, J=11.4, 2.1 Hz, 1H), 4.53-4.40 (m, 4H), 3.97-3.95 (m, 1H), 2.07-1.95 (m, 4H), 1.971.84 (m, 2H), 1.68-1.53 (m, 2H). ¹⁹F NMR (376 MHz, DMSO-D6) 8-92.03 (d, J=233.2 Hz, 1F), −99.39, −130.81 (dd, J=11.4, 1.5 Hz, 1F). UPLC-MS (Basic 4 min): rt=1.42 min, m/z (ES+) 366.3 [M+H]⁺.

### N-((1r,4r)-4-(((R)-1,1,1-Trifluoropropan-2-yl)amino)cyclohexyl)-5,6-dihydrobenzo[f]imidazo[1,5-d][1,4]oxazepine-10-carboxamide 74A

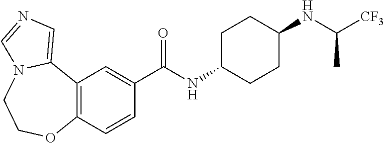 [SMILES: C[C@@H](N[C@H]1CC[C@H](NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and trans-N4-[(1R)-2,2,2-trifluoro-1-methyl-ethyl]cyclohexane-1,4-diamine according to General Procedure E to afford the desired product N-((1r,4r)-4-(((R)-1,1,1-trifluoropropan-2-yl)amino)cyclohexyl)-5,6-dihydrobenzo[f]imidazo[1,5-d][1,4]oxazepine-10-carboxamide (55 mg, 0.124 mmol, 29% yield) as an off-white solid. ¹H NMR (400 MHz, DMSO-D6) δ 8.23 (d, J=2.2 Hz, 1H), 8.17 (d, J=7.9 Hz, 1H), 7.77 (d, J=1.1 Hz, 1H), 7.65 (dd, J=8.6, 2.2 Hz, 1H), 7.61 (d, J=1.1 Hz, 1H), 7.03 (d, J=8.6 Hz, 1H), 4.52-4.39 (m, 4H), 3.78-3.66 (m, 1H), 3.37 (d, J=7.5 Hz, 1H), 2.03-1.73 (m, 5H), 1.41-1.30 (m, 2H), 1.19-1.09 (m, 5H). 1H not observed. ¹⁹F NMR (376 MHz, DMSO-D6) δ-75.95 (d, J=7.9 Hz). UPLC-MS (Basic 4 min): rt=1.48 min, m/z (ES+) 423.4 [M+H]⁺.

### N-((1s,4s)-4-((Tetrahydrofuran-2-yl)methoxy)cyclohexyl)-5,6-dihydrobenzo[f]imidazo[1,5-d][1,4]oxazepine-10-carboxamide 75A

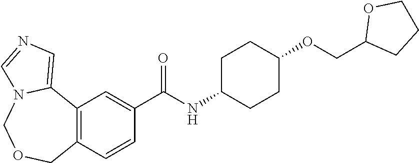 [SMILES: O=C(N[C@H]1CC[C@@H](OCC2CCCO2)CC1)c1ccc2c(c1)-c1cncn1COC2]

Prepared from lithium 5,6-dihydrobenzo[f]imidazo[1,5-d][1,4]oxazepine-10-carboxylate 4 and 4-((tetrahydrofuran-2-yl)methoxy)cyclohexan-1-amine according to General Procedure E to afford the desired product N-((1s,4s)-4-((tetrahydrofuran-2-yl)methoxy)cyclohexyl)-5,6-dihydrobenzo[f]imidazo[1,5-d][1,4]oxazepine-10-carboxamide (64 mg, 0.155 mmol, 18% yield) as a white solid. ¹H NMR (400 MHz, DMSO-D6) δ 8.26 (d, J=2.2 Hz, 1H), 8.25 (d, J=8.2 Hz, 1H), 7.77 (d, J=1.1 Hz, 1H), 7.68 (dd, J=8.5, 2.2 Hz, 1H), 7.62 (d, J=1.1 Hz, 1H), 7.03 (d, J=8.5 Hz, 1H), 4.51-4.39 (m, 4H), 3.93 (tt, J=7.0, 5.2 Hz, 1H), 3.82 (dt, J=7.7, 3.8 Hz, 1H), 3.74 (ddd, J=8.2, 7.0, 6.1 Hz, 1H), 3.62 (td, J=7.7, 6.4 Hz, 1H), 3.50 (d, J=8.7 Hz, 1H), 3.39-3.34 (m, 2H), 1.97-1.73 (m, 5H), 1.73-1.52 (m, 5H), 1.55-1.40 (m, 2H). UPLC-MS (Basic 6 min): rt=2.28 min, m/z (ES+) 412.3 [M+H]⁺.

### N-((1r,4r)-4-((Tetrahydrofuran-2-yl)methoxy)cyclohexyl)-5,6-dihydrobenzo[f]imidazo[1,5-d][1,4]oxazepine-10-carboxamide 76A

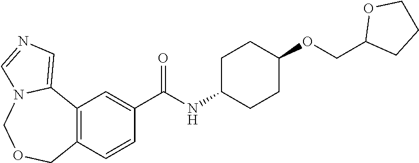 [SMILES: O=C(N[C@H]1CC[C@H](OCC2CCCO2)CC1)c1ccc2c(c1)-c1cncn1COC2]

Also isolated from the same reaction mixture was the other desired product N-((1r,4r)-4-((tetrahydrofuran-2-yl)methoxy)cyclohexyl)-5,6-dihydrobenzo[f]imidazo[1,5-d][1,4]oxazepine-10-carboxamide (10 mg, 0.0243 mmol, 3% yield) as a white solid. ¹H NMR (400 MHz, DMSO-D6) δ 8.23 (d, J=2.2 Hz, 1H), 8.18 (d, J=7.8 Hz, 1H), 7.77 (d, J=1.1 Hz, 1H), 7.65 (dd, J=8.6, 2.2 Hz, 1H), 7.60 (d, J=1.2 Hz, 1H), 7.03 (d, J=8.6 Hz, 1H), 4.51-4.39 (m, 4H), 3.94-3.83 (m, 1H), 3.81-3.67 (m, 2H), 3.65-3.56 (m, 1H), 3.43-3.34 (m, 2H), 3.30-3.20 (m, 1H), 2.05-1.95 (m, 2H), 1.94-1.69 (m, 5H), 1.59-1.46 (m, 1H), 1.44-1.31 (m, 2H), 1.29-1.12 (m, 2H). UPLC-MS (Basic 6 min): rt=2.17 min, m/z (ES+) 412.3 [M+H]⁺.

### N-((1s,4s)-4-(3,3-Difluoropropoxy)cyclohexyl)-5,6-dihydrobenzo[f]imidazo[1,5-d][1,4]oxazepine-10-carboxamide 77A

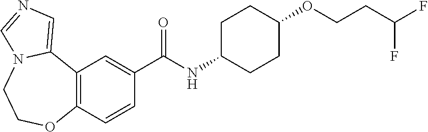 [SMILES: O=C(N[C@H]1CC[C@@H](OCCC(F)F)CC1)c1ccc2c(c1)-c1cncn1CCO2]

Prepared from lithium 5,6-dihydrobenzo[f]imidazo[1,5-d][1,4]oxazepine-10-carboxylate 4 and 4-(3,3-difluoropropoxy)cyclohexanamine according to General Procedure E to afford the desired product N-((1s,4s)-4-(3,3-difluoropropoxy)cyclohexyl)-5,6-dihydrobenzo[f]imidazo[1,5-d][1,4]oxazepine-10-carboxamide (61 mg, 0.150 mmol, 20% yield) as a white solid. ¹H NMR (400 MHz, DMSO-D6) δ 8.25 (d, J=2.2 Hz, 1H), 8.21 (d, J=7.8 Hz, 1H), 7.77 (s, 1H), 7.67 (dd, J=8.6, 2.2 Hz, 1H), 7.62 (s, 1H), 7.03 (d, J=8.5 Hz, 1H), 6.15 (tt, J=56.8, 4.8 Hz, 1H), 4.51-4.39 (m, 4H), 3.91-3.75 (m, 1H), 3.56-3.48 (m, 3H), 2.09 (ttd, J=17.3, 6.3, 4.8 Hz, 2H), 1.93-1.83 (m, 2H), 1.72-1.53 (m, 4H), 1.53-1.42 (m, 2H). ¹⁹F NMR (376 MHz, DMSO-D6) δ-116.10 (dt, J=56.8, 17.3 Hz, 2F). UPLC-MS (Basic 6 min): rt=2.52 min, m/z (ES+) 406.2 [M+H]⁺.

### N-((1r,4r)-4-(3,3-Difluoropropoxy)cyclohexyl)-5,6-dihydrobenzo[f]imidazo[1,5-d][1,4]oxazepine-10-carboxamide 78A

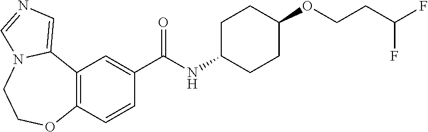 [SMILES: O=C(N[C@H]1CC[C@H](OCCC(F)F)CC1)c1ccc2c(c1)-c1cncn1CCO2]

Also isolated from the same reaction mixture was the other desired product N-((1r,4r)-4-(3,3-difluoropropoxy)cyclohexyl)-5,6-dihydrobenzo[f]imidazo[1,5-d][1,4]oxazepine-10-carboxamide (25 mg, 0.0616 mmol, 8% yield) as a white solid. ¹H NMR (400 MHz, DMSO-D6) δ 8.23 (d, J=2.2 Hz, 1H), 8.19 (d, J=7.7 Hz, 1H), 7.77 (d, J=1.1 Hz, 1H), 7.65 (dd, J=8.6, 2.1 Hz, 1H), 7.60 (d, J=1.1 Hz, 1H), 7.04 (d, J=8.6 Hz, 1H), 6.09 (tt, J=56.8, 4.7 Hz, 1H), 4.51-4.39 (m, 4H), 3.81-3.70 (m, 1H), 3.56 (t, J=6.3 Hz, 2H), 3.26 (td, J=10.5, 5.2 Hz, 1H), 2.14-1.97 (m, 4H), 1.91-1.84 (m, 2H), 1.43-1.36 (m, 2H), 1.26-1.20 (m, 2H). ¹⁹F NMR (376 MHz, DMSO-D6) δ-116.08 (dt, J=56.9, 17.4 Hz, 2F). UPLC-MS (Basic 6 min): rt=2.42 min, m/z (ES+) 406.3 [M+H]⁺.

### N-((1s,4s)-4-(3-Fluoropropoxy)cyclohexyl)-5,6-dihydrobenzo[f]imidazo[1,5-d][1,4]oxazepine-10-carboxamide 79A

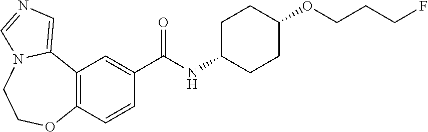 [SMILES: O=C(N[C@H]1CC[C@@H](OCCCF)CC1)c1ccc2c(c1)-c1cncn1CCO2]

Prepared from lithium 5,6-dihydrobenzo[f]imidazo[1,5-d][1,4]oxazepine-10-carboxylate 4 and 4-(3-fluoropropoxy)cyclohexanamine according to General Procedure E to afford the desired product N-((1s,4s)-4-(3-fluoropropoxy)cyclohexyl)-5,6-dihydrobenzo[f]imidazo[1,5-d][1,4]oxazepine-10-carboxamide (54 mg, 0.139 mmol, 16% yield) as a white solid. ¹H NMR (400 MHz, DMSO-D6) δ 8.26 (d, J=2.2 Hz, 1H), 8.23 (d, J=7.9 Hz, 1H), 7.77 (d, J=1.1 Hz, 1H), 7.68 (dd, J=8.5, 2.2 Hz, 1H), 7.62 (d, J=1.1 Hz, 1H), 7.03 (d, J=8.6 Hz, 1H), 4.66-4.38 (m, 6H), 3.91-3.75 (m, 1H), 3.53-3.43 (m, 3H), 1.97-1.80 (m, 4H), 1.73-1.53 (m, 4H), 1.53-1.42 (m, 2H). ¹⁹F NMR (376 MHz, DMSO-D6) δ-219.74 (tt, J=47.4, 25.4 Hz, 1F). UPLC-MS (Basic 6 min): rt=2.35 min, m/z (ES+) 388.2 [M+H]⁺.

### N-((1r,4r)-4-(3-Fluoropropoxy)cyclohexyl)-5,6-dihydrobenzo[f]imidazo[1,5-d][1,4]oxazepine-10-carboxamide 80A

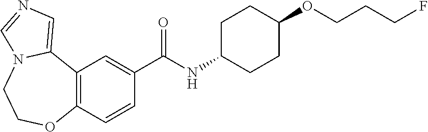 [SMILES: O=C(N[C@H]1CC[C@H](OCCCF)CC1)c1ccc2c(c1)-c1cncn1CCO2]

Also isolated from the same reaction mixture was the other desired product N-((1r,4r)-4-(3-fluoropropoxy)cyclohexyl)-5,6-dihydrobenzo[f]imidazo[1,5-d][1,4]oxazepine-10-carboxamide (21 mg, 0.0541 mmol, 6% yield) as a white solid. ¹H NMR (400 MHz, DMSO-D6) δ 8.23 (d, J=2.2 Hz, 1H), 8.19 (d, J=7.7 Hz, 1H), 7.77 (d, J=1.1 Hz, 1H), 7.65 (dd, J=8.5, 2.2 Hz, 1H), 7.60 (d, J=1.1 Hz, 1H), 7.04 (d, J=8.5 Hz, 1H), 4.62-4.36 (m, 6H), 3.82-3.70 (m, 1H), 3.51 (t, J=6.3 Hz, 2H), 3.29-3.16 (m, 1H), 2.02 (d, J=12.2 Hz, 2H), 1.95-1.76 (m, 4H), 1.43-1.35 (m, 2H), 1.30-1.17 (m, 2H). ¹⁹F NMR (376 MHz, DMSO-D6) δ-219.74 (tt, J=47.4, 25.6 Hz, 1F). UPLC-MS (Basic 6 min): rt=2.25 min, m/z (ES+) 388.2 [M+H]⁺.

### N-(2-(2-Methoxyethoxy)pyrimidin-5-yl)-5,6-dihydrobenzo[f]imidazo[1,5-d][1,4]oxazepine-10-carboxamide 81A

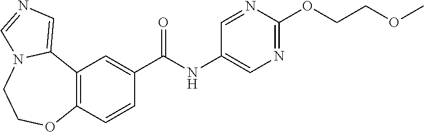 [SMILES: COCCOc1ncc(NC(=O)c2ccc3c(c2)-c2cncn2CCO3)cn1]

Prepared from lithium 5,6-dihydrobenzo[f]imidazo[1,5-d][1,4]oxazepine-10-carboxylate 4 and 2-(2-methoxyethoxy)pyrimidin-5-amine according to General Procedure E to afford the desired product N-(2-(2-methoxyethoxy)pyrimidin-5-yl)-5,6-dihydrobenzo[f]imidazo[1,5-d][1,4]oxazepine-10-carboxamide (13 mg, 0.0334 mmol, 8% yield) as an off-white solid. ¹H NMR (400 MHz, DMSO-D6) δ 10.39 (s, 1H), 8.86 (s, 2H), 8.38 (d, J=2.3 Hz, 1H), 7.79-7.71 (m, 2H), 7.62 (d, J=1.1 Hz, 1H), 7.10 (d, J=8.6 Hz, 1H), 4.51-4.40 (m, 4H), 4.41-4.35 (m, 2H), 3.67-3.60 (m, 2H), 3.27 (s, 3H). UPLC-MS (Basic 4 min): rt=1.18 min, m/z (ES+) 382.4 [M+H]⁺.

### N-((1r,4r)-4-((2,2-Difluorocyclobutyl)amino)cyclohexyl)-5,6-dihydrobenzo[f]imidazo[1,5-d][1,4]oxazepine-10-carboxamide 82A

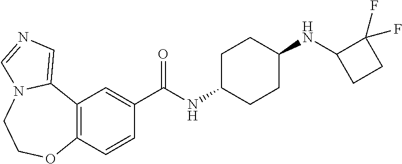 [SMILES: O=C(N[C@H]1CC[C@H](NC2CCC2(F)F)CC1)c1ccc2c(c1)-c1cncn1CCO2]

Prepared from lithium 5,6-dihydrobenzo[f]imidazo[1,5-d][1,4]oxazepine-10-carboxylate 4 and (1r,4r)-N1-(2,2-difluorocyclobutyl)cyclohexane-1,4-diamine according to General Procedure E to afford the desired product N-((1r,4r)-4-((2,2-difluorocyclobutyl)amino)cyclohexyl)-5,6-dihydrobenzo[f]imidazo[1,5-d][1,4]oxazepine-10-carboxamide (38 mg, 0.0900 mmol, 60% yield) as a white solid. ¹H NMR (400 MHz, DMSO-D6) δ 8.23 (d, J=2.2 Hz, 1H), 8.17 (d, J=7.9 Hz, 1H), 7.77 (d, J=1.1 Hz, 1H), 7.65 (dd, J=8.6, 2.2 Hz, 1H), 7.61 (d, J=1.2 Hz, 1H), 7.03 (d, J=8.6 Hz, 1H), 4.47 (dd, J=5.8, 2.5 Hz, 2H), 4.43 (dt, J=6.0, 1.8 Hz, 2H), 3.83-3.68 (m, 1H), 3.62 (s, 1H), 2.48-2.38 (m, 1H), 2.25-1.98 (m, 4H), 1.96-1.72 (m, 4H), 1.45-1.26 (m, 3H), 1.21-1.04 (m, 2H). ¹⁹F NMR (376 MHz, DMSO-D6) δ-82.76 (d, J=188.2 Hz, 1F), −119.15 (ddd, J=189.0, 34.7, 18.8 Hz, 1F). UPLC-MS (Basic 4 min): rt=1.36 min, m/z (ES+) 417.3 [M+H]⁺.

### N-((1r,4r)-4-((2-Fluoro-2-methylpropyl)amino)cyclohexyl)-5,6-dihydrobenzo[f]imidazo[1,5-d][1,4]oxazepine-10-carboxamide 83A

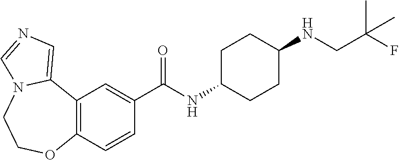 [SMILES: CC(C)(F)CN[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and (1r,4r)-N1-(2-fluoro-2-methylpropyl)cyclohexane-1,4-diamine according to General Procedure E to afford the desired product N-((1r,4r)-4-((2-fluoro-2-methylpropyl)amino)cyclohexyl)-5,6-dihydrobenzo[f]imidazo[1,5-d][1,4]oxazepine-10-carboxamide (48 mg, 0.118 mmol, 40% yield) as a white solid. ¹H NMR (400 MHz, DMSO-D6) δ 8.23 (d, J=2.2 Hz, 1H), 8.18 (d, J=7.9 Hz, 1H), 7.77 (d, J=1.1 Hz, 1H), 7.66 (dd, J=8.6, 2.2 Hz, 1H), 7.61 (d, J=1.1 Hz, 1H), 7.03 (d, J=8.6 Hz, 1H), 4.52-4.45 (m, 2H), 4.44-4.39 (m, 2H), 3.84-3.66 (m, 1H), 2.71-2.60 (m, 2H), 2.39-2.26 (m, 1H), 2.00-1.89 (m, 2H), 1.88-1.78 (m, 2H), 1.44-1.30 (m, 2H), 1.33 (s, 3H), 1.27 (s, 3H), 1.17-1.01 (m, 2H). 1H not observed. ¹⁹F NMR (376 MHz, DMSO-D6) δ-140.10--140.67 (m, 1F). UPLC-MS (Basic 4 min): rt=1.36 min, m/z (ES+) 401.4 [M+H]⁺.

### N-(3,3-Difluorocyclobutyl)-5,6-dihydrobenzo[f]imidazo[1,5-d][1,4]oxazepine-10-carboxamide 84A

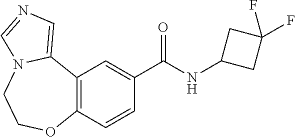 [SMILES: O=C(NC1CC(F)(F)C1)c1ccc2c(c1)-c1cncn1CCO2]

Prepared from lithium 5,6-dihydrobenzo[f]imidazo[1,5-d][1,4]oxazepine-10-carboxylate 4 and 3,3-difluorocyclobutanamine hydrochloride according to General Procedure E to afford the desired product N-(3,3-difluorocyclobutyl)-5,6-dihydrobenzo[f]imidazo[1,5-d][1,4]oxazepine-10-carboxamide (84 mg, 0.260 mmol, 62% yield) as an off-white solid. 1H NMR (400 MHz, DMSO-D6) δ 8.74 (d, J=6.6 Hz, 1H), 8.23 (d, J=2.2 Hz, 1H), 7.75 (d, J=1.1 Hz, 1H), 7.63 (dd, J=8.6, 2.2 Hz, 1H), 7.57 (d, J=1.1 Hz, 1H), 7.03 (d, J=8.5 Hz, 1H), 4.50-4.37 (m, 4H), 4.29-4.18 (m, 1H), 3.00-2.85 (m, 2H), 2.82-2.64 (m, 2H). ¹⁹F NMR (376 MHz, DMSO-D6) δ-80.89--82.03 (m, 1F), −96.39--97.74 (m, 1F). UPLC-MS (Basic 4 min): rt=1.36 min, m/z (ES+) 401.4 [M+H]⁺.

### N-((1r,4r)-4-(3-(Methylsulfonyl) propoxy)cyclohexyl)-5,6-dihydrobenzo[f]imidazo[1,5-d][1,4]oxazepine-10-carboxamide 85A

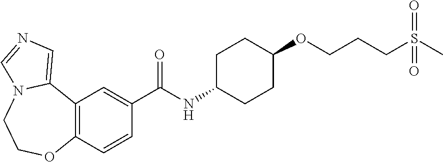 [SMILES: CS(=O)(=O)CCCO[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and 4-(3-methylsulfonylpropoxy)cyclohexanamine according to General Procedure E to afford the desired product N-((1r,4r)-4-(3-(methylsulfonyl) propoxy)cyclohexyl)-5,6-dihydrobenzo[f]imidazo[1,5-d][1,4]oxazepine-10-carboxamide (14 mg, 0.155 mmol, 4% yield) as a white solid. ¹H NMR (400 MHz, DMSO-D6) δ 8.23 (d, J=2.2 Hz, 1H), 8.19 (d, J=7.8 Hz, 1H), 7.77 (d, J=1.0 Hz, 1H), 7.65 (dd, J=8.5, 2.2 Hz, 1H), 7.60 (d, J=1.1 Hz, 1H), 7.04 (d, J=8.6 Hz, 1H), 4.51-4.39 (m, 4H), 3.82-3.70 (m, 1H), 3.52 (t, J=6.2 Hz, 2H), 3.23 (td, J=10.4, 5.1 Hz, 1H), 3.17-3.09 (m, 2H), 2.98 (s, 3H), 2.06-1.98 (m, 2H), 1.95-1.84 (m, 4H), 1.46-1.32 (m, 2H), 1.30-1.18 (m, 2H). UPLC-MS (Basic 6 min): rt=1.80 min, m z (ES+) 448.3 [M+H]⁺.

### N-((1s,4s)-4-(3-(Methylsulfonyl) propoxy)cyclohexyl)-5,6-dihydrobenzo[f]imidazo[1,5-d][1,4]oxazepine-10-carboxamide 86A

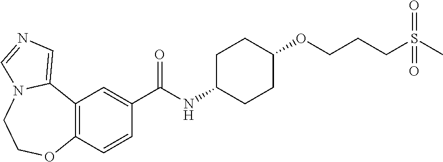 [SMILES: CS(=O)(=O)CCCO[C@H]1CC[C@@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and 4-(3-methylsulfonylpropoxy)cyclohexanamine according to General Procedure E to afford the desired product N-((1s,4s)-4-(3-(methylsulfonyl) propoxy)cyclohexyl)-5,6-dihydrobenzo[f]imidazo[1,5-d][1,4]oxazepine-10-carboxamide (6.5 mg, 0.0145 mmol, 1% yield) as a white solid. ¹H NMR (400 MHz, DMSO-D6) δ 8.26 (d, J=2.2 Hz, 1H), 8.24 (d, J=7.9 Hz, 1H), 7.77 (d, J=1.1 Hz, 1H), 7.68 (dd, J=8.5, 2.2 Hz, 1H), 7.63 (d, J=1.1 Hz, 1H), 7.03 (d, J=8.6 Hz, 1H), 4.51-4.39 (m, 4H), 3.90-3.76 (m, 1H), 3.48 (t, J=6.3 Hz, 3H), 3.22-3.13 (m, 2H), 2.99 (d, J=0.7 Hz, 3H), 1.99-1.83 (m, 4H), 1.72-1.54 (m, 4H), 1.52-1.44 (m, 2H). UPLC-MS (Basic 6 min): rt=1.82 min, m/z (ES+) 448.4 [M+H]⁺.

### N-((1r,4r)-4-(((S)-1,1,1-Trifluoropropan-2-yl)amino)cyclohexyl)-5,6-dihydrobenzo[f]imidazo[1,5-d][1,4]oxazepine-10-carboxamide 87A

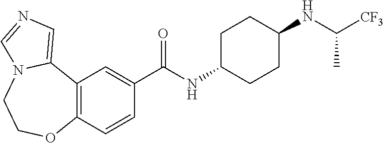 [SMILES: C[C@H](N[C@H]1CC[C@H](NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and trans-N4-[(1S)-2,2,2-trifluoro-1-methyl-ethyl]cyclohexane-1,4-diamine according to General Procedure E to afford the desired product N-((1r,4r)-4-(((S)-1,1,1-trifluoropropan-2-yl)amino)cyclohexyl)-5,6-dihydrobenzo[f]imidazo[1,5-d][1,4]oxazepine-10-carboxamide (49 mg, 0.115 mmol, 33% yield) as a white solid. ¹H NMR (400 MHz, DMSO-D6) δ 8.23 (d, J=2.2 Hz, 1H), 8.18 (d, J=7.9 Hz, 1H), 7.77 (d, J=1.1 Hz, 1H), 7.65 (dd, J=8.6, 2.2 Hz, 1H), 7.61 (d, J=1.1 Hz, 1H), 7.03 (d, J=8.5 Hz, 1H), 4.52-4.46 (m, 2H), 4.43 (dt, J=5.7, 1.6 Hz, 2H), 3.73 (dtd, J=11.5, 7.7, 3.9 Hz, 1H), 2.02-1.93 (m, 1H), 1.91-1.72 (m, 5H), 1.44-1.31 (m, 2H), 1.16 (d, J=6.7 Hz, 3H), 1.15-1.02 (m, 2H). 1H not observed. ¹⁹F NMR (376 MHz, DMSO-D6) δ-75.94 (d, J=8.0 Hz). UPLC-MS (Basic 4 min): rt=1.49 min, m z (ES+) 423.3 [M+H]⁺.

### N-((1r,4r)-4-((1,1,1-Trifluoro-2-methylpropan-2-yl)amino)cyclohexyl)-5,6-dihydrobenzo[f]imidazo[1,5-d][1,4]oxazepine-10-carboxamide 88A

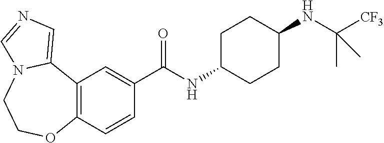 [SMILES: CC(C)(N[C@H]1CC[C@H](NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and trans-N4-(2,2,2-trifluoro-1,1-dimethyl-ethyl)cyclohexane-1,4-diamine according to General Procedure E to afford the desired product N-((1r,4r)-4-((1,1,1-trifluoro-2-methylpropan-2-yl)amino)cyclohexyl)-5,6-dihydrobenzo[f]imidazo[1,5-d][1,4]oxazepine-10-carboxamide (20 mg, 0.0465 mmol, 31% yield) as a white solid. ¹H NMR (400 MHz, DMSO-D6) δ 8.22 (d, J=2.2 Hz, 1H), 8.15 (d, J=7.8 Hz, 1H), 7.77 (d, J=1.1 Hz, 1H), 7.65 (dd, J=8.6, 2.2 Hz, 1H), 7.60 (d, J=1.1 Hz, 1H), 7.03 (d, J=8.6 Hz, 1H), 4.51-4.45 (m, 2H), 4.43 (dt, J=5.6, 1.6 Hz, 2H), 3.88-3.58 (m, 1H), 2.82-2.57 (m, 1H), 1.89-1.70 (m, 5H), 1.48-1.31 (m, 2H), 1.28-1.11 (m, 2H), 1.21 (s, 6H). ¹⁹F NMR (376 MHz, DMSO-D6) δ-78.33 (s). UPLC-MS (Basic 4 min): rt=1.65 min, m/z (ES+) 437.3 [M+H]⁺.

### N-((1s,4s)-4-(3,3,3-Trifluoropropoxy)cyclohexyl)-5,6-dihydrobenzo[f]imidazo[1,5-d][1,4]oxazepine-10-carboxamide 89A

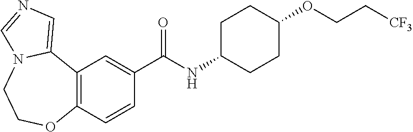 [SMILES: O=C(N[C@H]1CC[C@@H](OCCC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and 4-(3,3,3-trifluoropropoxy)cyclohexanamine according to General Procedure E to afford the desired product N-((1s,4s)-4-(3,3,3-trifluoropropoxy)cyclohexyl)-5,6-dihydrobenzo[f]imidazo[1,5-d][1,4]oxazepine-10-carboxamide (36 mg, 0.0849 mmol, 18% yield) as a white solid. ¹H NMR (400 MHz, Acetonitrile-D3) δ 8.14 (d, J=2.3 Hz, 1H), 7.59 (dd, J=8.6, 2.3 Hz, 1H), 7.54 (dd, J=8.9, 1.1 Hz, 2H), 7.03 (d, J=8.5 Hz, 1H), 6.87 (d, J=7.7 Hz, 1H), 4.48-4.37 (m, 4H), 3.94-3.85 (m, 1H), 3.65 (td, J=6.3, 1.3 Hz, 2H), 3.54 (tt, J=4.3, 2.7 Hz, 1H), 2.53-2.37 (m, 2H), 1.92-1.83 (m, 2H), 1.74-1.64 (m, 4H), 1.64-1.51 (m, 2H). ¹⁹F NMR (376 MHz, DMSO-D6) δ-62.78 (t, J=11.5 Hz, 3F). UPLC-MS (Basic 6 min): rt=2.73 min, m/z (ES+) 424.3 [M+H]⁺.

### N-((1r,4r)-4-(3,3,3-Trifluoropropoxy)cyclohexyl)-5,6-dihydrobenzo[f]imidazo[1,5-d][1,4]oxazepine-10-carboxamide 90A

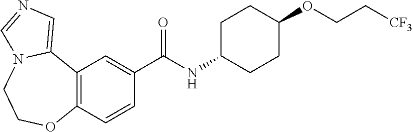 [SMILES: O=C(N[C@H]1CC[C@H](OCCC(F)(F)F)CC1)c1ccc2c(c1)-c1cncn1CCO2]

Also isolated from the same reaction mixture was the other desired product N-((1r,4r)-4-(3,3,3-trifluoropropoxy)cyclohexyl)-5,6-dihydrobenzo[f]imidazo[1,5-d][1,4]oxazepine-10-carboxamide (11 mg, 0.0257 mmol, 6% yield) as a white solid. ¹H NMR (400 MHz, Acetonitrile-D3) δ 8.13 (d, J=2.3 Hz, 1H), 7.58 (dd, J=8.6, 2.2 Hz, 1H), 7.54 (dd, J=8.4, 1.1 Hz, 2H), 7.03 (d, J=8.5 Hz, 1H), 6.82 (d, J=7.9 Hz, 1H), 4.43 (s, 4H), 3.88-3.78 (m, 1H), 3.68 (t, J=6.3 Hz, 2H), 3.36-3.25 (m, 1H), 2.46-2.38 (m, 2H), 2.08-1.96 (m, 4H), 1.47-1.25 (m, 4H). ¹⁹F NMR (376 MHz, DMSO-D6) δ-62.83 (t, J=11.2 Hz, 3F). UPLC-MS (Basic 6 min): rt=2.65 min, m/z (ES+) 424.3 [M+H]⁺.

### 8,11-Difluoro-N-((1r,4r)-4-(2-methoxyethoxy)cyclohexyl)-5,6-dihydrobenzo[f]imidazo[1,5-d][1,4]oxazepine-10-carboxamide 91A

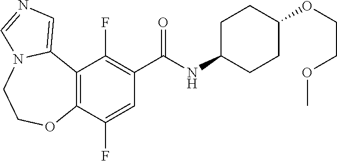 [SMILES: COCCO[C@H]1CC[C@H](NC(=O)c2cc(F)c3c(c2F)-c2cncn2CCO3)CC1]

Prepared from lithium 8,11-difluoro-5,6-dihydrobenzo[f]imidazo[1,5-d][1,4]oxazepine-10-carboxylate and trans-4-(2-methoxyethoxy)cyclohexanamine according to General Procedure E to afford the desired product 8,11-difluoro-N-((1r,4r)-4-(2-methoxyethoxy)cyclohexyl)-5,6-dihydrobenzo[f]imidazo[1,5-d][1,4]oxazepine-10-carboxamide (36 mg, 0.0853 mmol, 29% yield) as a white solid. ¹H NMR (400 MHz, DMSO-D6) δ 8.25 (d, J=7.7 Hz, 1H), 7.92 (s, 1H), 7.43-7.33 (m, 2H), 4.61-4.54 (m, 2H), 4.51-4.44 (m, 2H), 3.75-3.64 (m, 1H), 3.55-3.48 (m, 2H), 3.45-3.38 (m, 2H), 3.26-3.19 (m, 4H), 2.02-1.95 (m, 2H), 1.91-1.83 (m, 2H), 1.39-1.16 (m, 4H). ¹⁹F NMR (376 MHz, DMSO-D6) δ-115.26--115.40 (m, F), −135.36 (dd, J=15.5, 10.3 Hz, F). UPLC-MS (Basic 4 min): rt=1.30 min, m z (ES+) 422.3 [M+H]⁺.

### (R)—N-(3,3-Difluorocyclopentyl)-5,6-dihydrobenzo[f]imidazo[1,5-d][1,4]oxazepine-10-carboxamide 92A

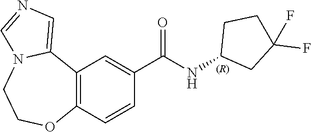

Isolated by chiral separation of N-(3,3-difluorocyclopentyl)-5,6-dihydrobenzo[f]imidazo[1,5-d][1,4]oxazepine-10-carboxamide according to Purification Method A to afford the desired product (R)—N-(3,3-difluorocyclopentyl)-5,6-dihydrobenzo[f]imidazo[1,5-d][1,4]oxazepine-10-carboxamide (21 mg, 0.0635 mmol, 27% yield) as an off-white solid. ¹H NMR (400 MHz, DMSO-D6) δ 8.49 (d, J=7.2 Hz, 1H), 8.21 (d, J=2.2 Hz, 1H), 7.74 (d, J=1.1 Hz, 1H), 7.63 (dd, J=8.6, 2.2 Hz, 1H), 7.57 (d, J=1.1 Hz, 1H), 7.02 (d, J=8.6 Hz, 1H), 4.48-4.32 (m, 5H), 2.32-2.15 (m, 1H), 2.21-1.96 (m, 3H), 1.856-1.81 (m, 1H). ¹H not observed. ¹⁹F NMR (376 MHz, DMSO-D6) δ-87.68 (s), −87.75 (s). UPLC-MS (Basic 4 min): rt=1.30 min, m/z (ES+) 334.3 [M+H]⁺.

### (S)—N-(3,3-Difluorocyclopentyl)-5,6-dihydrobenzo[f]imidazo[1,5-d][1,4]oxazepine-10-carboxamide 93A

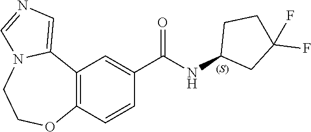

Isolated by chiral separation of N-(3,3-difluorocyclopentyl)-5,6-dihydrobenzo[f]imidazo[1,5-d][1,4]oxazepine-10-carboxamide according to Purification Method A to afford the desired product(S)—N-(3,3-difluorocyclopentyl)-5,6-dihydrobenzo[f]imidazo[1,5-d][1,4]oxazepine-10-carboxamide (26 mg, 0.0791 mmol, 33% yield) as an off-white solid. 1H NMR (400 MHz, DMSO-D6) δ 8.49 (d, J=7.2 Hz, 1H), 8.21 (d, J=2.2 Hz, 1H), 7.74 (d, J=1.1 Hz, 1H), 7.63 (dd, J=8.6, 2.2 Hz, 1H), 7.57 (d, J=1.1 Hz, 1H), 7.02 (d, J=8.6 Hz, 1H), 4.48-4.32 (m, 5H), 2.30-1.96 (m, 4H), 1.88-1.72 (m, 1H). 1H not observed. ¹⁹F NMR (376 MHz, DMSO-D6) δ-87.68 (s), −87.75 (s). UPLC-MS (Basic 4 min): rt=1.30 min, m/z (ES+) 334.3 [M+H]⁺.

### 8-Chloro-N-((1r,4r)-4-(2-methoxyethoxy)cyclohexyl)-5,6-dihydrobenzo[f]imidazo[1,5-d][1,4]oxazepine-10-carboxamide 94A

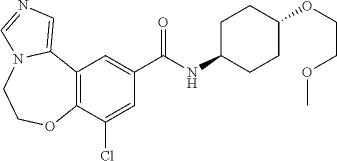 [SMILES: COCCO[C@H]1CC[C@H](NC(=O)c2cc(Cl)c3c(c2)-c2cncn2CCO3)CC1]

Prepared from lithium 8-chloro-5,6-dihydrobenzo[f]imidazo[1,5-d][1,4]oxazepine-10-carboxylate and trans-4-(2-methoxyethoxy)cyclohexanamine according to General Procedure E to afford the desired product 8-chloro-N-((1r,4r)-4-(2-methoxyethoxy)cyclohexyl)-5,6-dihydrobenzo[f]imidazo[1,5-d][1,4]oxazepine-10-carboxamide (77.7 mg, 0.185 mmol, 56% yield) as a white solid. ¹H NMR (400 MHz, DMSO-D6) δ 8.30 (d, J=7.7 Hz, 1H), 8.18 (d, J=2.2 Hz, 1H), 7.83 (s, 2H), 7.62 (d, J=1.1 Hz, 1H), 4.59-4.47 (m, 4H), 3.82-3.69 (m, OH), 3.53 (dd, J=5.9, 3.8 Hz, 2H), 3.42 (dd, J=5.8, 3.8 Hz, 2H), 3.24 (s, 3H), 2.05-1.98 (m, 2H), 1.90-1.82 (m, 2H), 1.44-1.31 (m, 2H), 1.29-1.16 (m, 2H). 1H not observed. UPLC-MS (Basic 4 min): rt=1.39 min, m/z (ES+) 420.4, 422.4 [M+H]⁺.

### 8,9-Difluoro-N-((1r,4r)-4-(2-methoxyethoxy)cyclohexyl)-5,6-dihydrobenzo[f]imidazo[1,5-d][1,4]oxazepine-10-carboxamide 95A

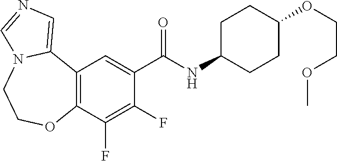 [SMILES: COCCO[C@H]1CC[C@H](NC(=O)c2cc3c(c(F)c2F)OCCn2cncc2-3)CC1]

Prepared from lithium 8,9-difluoro-5,6-dihydrobenzo[f]imidazo[1,5-d][1,4]oxazepine-10-carboxylate and trans-4-(2-methoxyethoxy)cyclohexanamine according to General Procedure E to afford the desired product 8,9-difluoro-N-((1r,4r)-4-(2-methoxyethoxy)cyclohexyl)-5,6-dihydrobenzo[f]imidazo[1,5-d][1,4]oxazepine-10-carboxamide (36 mg, 0.0853 mmol, 29% yield) as a white solid. ¹H NMR (400 MHz, DMSO-D6) δ 8.23 (d, J=7.7 Hz, 1H), 7.76 (d, J=1.1 Hz, 1H), 7.67 (dd, J=7.4, 2.3 Hz, 1H), 7.52 (d, J=1.1 Hz, 1H), 4.56-4.45 (m, 4H), 3.73-3.61 (m, 1H), 3.50-3.47 (m, 2H), 3.39-3.36 (m, 2H), 3.21-3.17 (m, 4H), 1.97-1.94 (m, 2H), 1.86-1.83 (m, 2H), 1.36-1.12 (m, 4H). ¹⁹F NMR (376 MHz, DMSO-D6) δ-155.34 (s, F), −155.40 (s, F). UPLC-MS (Basic 4 min): rt=1.39 min, m/z (ES+) 420.3 [M−H]⁺.

### N-(3,3-Difluorocyclohexyl)-5,6-dihydrobenzo[f]imidazo[1,5-d][1,4]oxazepine-10-carboxamide 96A

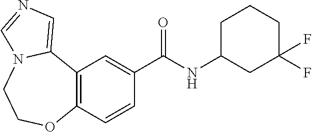 [SMILES: O=C(NC1CCCC(F)(F)C1)c1ccc2c(c1)-c1cncn1CCO2]

Prepared from lithium 5,6-dihydrobenzo[f]imidazo[1,5-d][1,4]oxazepine-10-carboxylate 4 and 3,3-difluorocyclohexanamine hydrochloride according to General Procedure E to afford the desired product N-(3,3-difluorocyclohexyl)-5,6-dihydrobenzo[f]imidazo[1,5-d][1,4]oxazepine-10-carboxamide (280 mg, 0.806 mmol, 95% yield) as a white solid. ¹H NMR (400 MHz, DMSO-D6) δ 8.22 (d, J=2.3 Hz, 1H), 8.21 (s, 1H), 7.74 (d, J=1.1 Hz, 1H), 7.66 (dd, J=8.5, 2.2 Hz, 1H), 7.57 (d, J=1.1 Hz, 1H), 7.05 (d, J=8.5 Hz, 1H), 4.52-4.41 (m, 4H), 4.12-3.92 (m, 1H), 2.38-2.22 (m, 1H), 2.11-1.98 (m, 1H), 1.98-1.64 (m, 4H), 1.56-1.36 (m, 2H). ¹⁹F NMR (376 MHz, DMSO-D6) δ-85.52 (br s, 1F), −86.15 (br s, 1F). UPLC-MS (Basic 6 min): rt=2.21 min, m/z (ES+) 348.3 [M+H]⁺.

### N-(2,3-Dihydro-[1,4]dioxino[2,3-b]pyridin-7-yl)-8-fluoro-5,6-dihydrobenzo[f]imidazo[1,5-d][1,4]oxazepine-10-carboxamide 97A

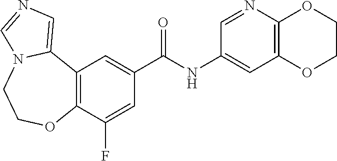 [SMILES: O=C(Nc1cnc2c(c1)OCCO2)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2,3-dihydro-[1,4]dioxino[2,3-b]pyridin-7-amine according to General Procedure E to afford the desired product N-(2,3-dihydro-[1,4]dioxino[2,3-b]pyridin-7-yl)-8-fluoro-5,6-dihydrobenzo[f]imidazo[1,5-d][1,4]oxazepine-10-carboxamide (25 mg, 0.0653 mmol, 17% yield) as a beige solid. ¹H NMR (400 MHz, DMSO-D6) δ 10.29 (s, 1H), 8.25-8.19 m, 1H), 8.14-8.04 (m, 1H), 7.84 (d, J=1.1 Hz, 1H), 7.78-7.72 (m, 1H), 7.70 (d, J=1.1 Hz, 1H), 7.67 (d, J=2.1 Hz, 1H), 4.56 (s, 4H), 4.45-4.33 (m, 2H), 4.34-4.12 (m, 2H). ¹⁹F NMR (376 MHz, DMSO-D6) δ-130.36 (d, J=10.7 Hz, 1F). UPLC-MS (Basic 4 min): rt=1.23 min, m/z (ES+) 383.2 [M+H]⁺.

### N-(4-Methyl-3,4-dihydro-2H-pyrido[3,2-b][1,4]oxazin-7-yl)-5,6-dihydrobenzo[f]imidazo[1,5-d][1,4]oxazepine-10-carboxamide 98A

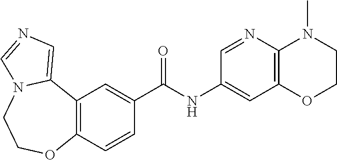 [SMILES: CN1CCOc2cc(NC(=O)c3ccc4c(c3)-c3cncn3CCO4)cnc21]

Prepared from lithium 5,6-dihydrobenzo[f]imidazo[1,5-d][1,4]oxazepine-10-carboxylate 4 and 4-methyl-2,3-dihydropyrido[3,2-b][1,4]oxazin-7-amine according to General Procedure E to afford the desired product N-(4-methyl-3,4-dihydro-2H-pyrido[3,2-b][1,4]oxazin-7-yl)-5,6-dihydrobenzo[f]imidazo[1,5-d][1,4]oxazepine-10-carboxamide (46 mg, 0.121 mmol, 29% yield) as an off-white solid. ¹H NMR (400 MHz, DMSO-D6) δ 10.02 (s, 1H), 8.36 (d, J=2.3 Hz, 1H), 8.04 (d, J=2.1 Hz, 1H), 7.79 (d, J=1.1 Hz, 1H), 7.75 (dd, J=8.6, 2.2 Hz, 1H), 7.66 (d, J=1.1 Hz, 1H), 7.38 (d, J=2.2 Hz, 1H), 7.10 (d, J=8.6 Hz, 1H), 4.55-4.48 (m, 2H), 4.48-4.43 (m, 2H), 4.24 (dd, J=5.2, 3.9 Hz, 2H), 3.42-3.37 (m, 2H), 3.00 (s, 3H). UPLC-MS (Basic 6 min): rt=1.99 min, m/z (ES+) 378.4 [M+H]⁺.

### N-(2-(3,3-Difluoroazetidin-1-yl)pyrimidin-5-yl)-5,6-dihydrobenzo[f]imidazo[1,5-d][1,4]oxazepine-10-carboxamide 99A

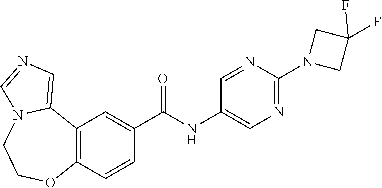 [SMILES: O=C(Nc1cnc(N2CC(F)(F)C2)nc1)c1ccc2c(c1)-c1cncn1CCO2]

Prepared from lithium 5,6-dihydrobenzo[f]imidazo[1,5-d][1,4]oxazepine-10-carboxylate 4 and 2-(3,3-difluoroazetidin-1-yl)pyrimidin-5-amine according to General Procedure E to afford the desired product N-(2-(3,3-difluoroazetidin-1-yl)pyrimidin-5-yl)-5,6-dihydrobenzo[f]imidazo[1,5-d][1,4]oxazepine-10-carboxamide (102 mg, 0.254 mmol, 60% yield) as an off-white solid. ¹H NMR (400 MHz, DMSO-D6) δ 10.28 (s, 1H), 8.77 (s, 2H), 8.41 (d, J=2.2 Hz, 1H), 7.82-7.74 (m, 2H), 7.66 (s, 1H), 7.13 (d, J=8.5 Hz, 1H), 4.52-4.44 (m, 8H). ¹⁹F NMR (376 MHz, DMSO-D6) δ-98.81 (p, J=12.6 Hz, 2F). UPLC-MS (Basic 6 min): rt=2.11 min, m/z (ES+) 399.4 [M+H]⁺.

### N-(2-((2,2,2-Trifluoroethyl)amino)pyrimidin-5-yl)-5,6-dihydrobenzo[f]imidazo[1,5-d][1,4]oxazepine-10-carboxamide 100A

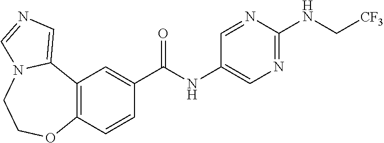 [SMILES: O=C(Nc1cnc(NCC(F)(F)F)nc1)c1ccc2c(c1)-c1cncn1CCO2]

Prepared from lithium 5,6-dihydrobenzo[f]imidazo[1,5-d][1,4]oxazepine-10-carboxylate 4 and 2-(2,2,2-trifluoroethyl)pyrimidine-2,5-diamine according to General Procedure E to afford the desired product N-(2-((2,2,2-trifluoroethyl)amino)pyrimidin-5-yl)-5,6-dihydrobenzo[f]imidazo[1,5-d][1,4]oxazepine-10-carboxamide (130 mg, 0.318 mmol, 75% yield) as a grey solid. ¹H NMR (400 MHz, DMSO-D6) δ 10.19 (s, 1H), 8.64 (s, 2H), 8.40 (s, 1H), 7.84-7.65 (m, 4H), 7.13 (d, J=8.6 Hz, 1H), 4.54-4.45 (m, 4H), 4.20-4.07 (m, 2H). 19F NMR (376 MHz, DMSO-D6) δ-70.85 (t, J=9.6 Hz, 3F). UPLC-MS (Basic 6 min): rt=2.15 min, m/z (ES+) 405.4 [M+H]⁺.

### N-(1-Methyl-2,3-dihydro-1H-pyrido[2,3-b][1,4]oxazin-7-yl)-5,6-dihydrobenzo[f]imidazo[1,5-d][1,4]oxazepine-10-carboxamide 101A

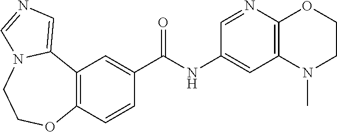 [SMILES: CN1CCOc2ncc(NC(=O)c3ccc4c(c3)-c3cncn3CCO4)cc21]

Prepared from lithium 5,6-dihydrobenzo[f]imidazo[1,5-d][1,4]oxazepine-10-carboxylate 4 and 1-methyl-2,3-dihydropyrido[2,3-b][1,4]oxazin-7-amine trihydrochloride according to General Procedure E to afford the desired product N-(1-methyl-2,3-dihydro-1H-pyrido[2,3-b][1,4]oxazin-7-yl)-5,6-dihydrobenzo[f]imidazo[1,5-d][1,4]oxazepine-10-carboxamide (42 mg, 0.111 mmol, 26% yield) as a beige solid. ¹H NMR (400 MHz, DMSO-D6) δ 10.09 (s, 1H), 8.38 (d, J=2.3 Hz, 1H), 7.84-7.81 (m, 1H), 7.79 (d, J=1.1 Hz, 1H), 7.77 (dd, J=8.6, 2.2 Hz, 1H), 7.66 (d, J=1.1 Hz, 1H), 7.44-7.39 (m, 1H), 7.11 (d, J=8.6 Hz, 1H), 4.54-4.43 (m, 4H), 4.37-4.30 (m, 2H), 3.28-3.25 (m, 2H), 2.85 (s, 3H). UPLC-MS (Basic 4 min): rt=1.18 min, m/z (ES+) 378.4 [M+H]⁺.

### N-(2-(3-Fluoropropoxy)pyrimidin-5-yl)-5,6-dihydrobenzo[f]imidazo[1,5-d][1,4]oxazepine-10-carboxamide 102A

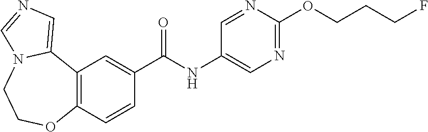 [SMILES: O=C(Nc1cnc(OCCCF)nc1)c1ccc2c(c1)-c1cncn1CCO2]

Prepared from lithium 5,6-dihydrobenzo[f]imidazo[1,5-d][1,4]oxazepine-10-carboxylate 4 and 2-(3-fluoropropoxy)pyrimidin-5-amine according to General Procedure E to afford the desired product N-(2-(3-fluoropropoxy)pyrimidin-5-yl)-5,6-dihydrobenzo[f]imidazo[1,5-d][1,4]oxazepine-10-carboxamide (77 mg, 0.201 mmol, 38% yield) as an off-white solid. ¹H NMR (400 MHz, DMSO-D6) δ 10.43 (s, 1H), 8.91 (d, J=0.8 Hz, 2H), 8.42 (d, J=2.2 Hz, 1H), 7.82-7.75 (m, 2H), 7.66 (t, J=1.0 Hz, 1H), 7.14 (dd, J=8.6, 0.8 Hz, 1H), 4.61 (dt, J=47.3, 5.9 Hz, 2H), 4.53-4.45 (m, 4H), 4.40 (t, J=6.4 Hz, 2H), 2.14 (dtt, J=25.2, 6.2, 6.2 Hz, 2H). ¹⁹F NMR (376 MHz, DMSO-D6) δ-220.17 (tt, J=47.3, 25.9 Hz, 1F). UPLC-MS (Basic 6 min): rt=2.06 min, m/z (ES+) 384.3 [M+H]⁺.

### N-(2-(3,3-Difluoropropoxy)pyrimidin-5-yl)-5,6-dihydrobenzo[f]imidazo[1,5-d][1,4]oxazepine-10-carboxamide 103A

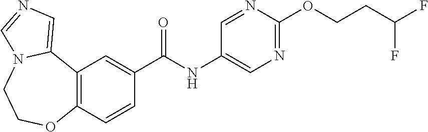 [SMILES: O=C(Nc1cnc(OCCC(F)F)nc1)c1ccc2c(c1)-c1cncn1CCO2]

Prepared from lithium 5,6-dihydrobenzo[f]imidazo[1,5-d][1,4]oxazepine-10-carboxylate 4 and 2-(3,3-difluoropropoxy)pyrimidin-5-amine according to General Procedure E to afford the desired product N-(2-(3,3-difluoropropoxy)pyrimidin-5-yl)-5,6-dihydrobenzo[f]imidazo[1,5-d][1,4]oxazepine-10-carboxamide (43 mg, 0.107 mmol, 25% yield) as a white solid. ¹H NMR (400 MHz, DMSO-D6) δ 10.44 (s, 1H), 8.92 (s, 2H), 8.42 (d, J=2.2 Hz, 1H), 7.81-7.79 (m, 1H), 7.78 (dd, J=2.4, 1.7 Hz, 1H), 7.66 (d, J=1.1 Hz, 1H), 7.14 (d, J=8.6 Hz, 1H), 6.26 (tt, J=56.3, 4.6 Hz, 1H), 4.55-4.47 (m, 2H), 4.51-4.43 (m, 2H), 4.45 (t, J=6.4 Hz, 2H), 2.45-2.26 (m, 2H). ¹⁹F NMR (376 MHz, DMSO-D6) δ-115.76 (dt, J=56.3, 17.4 Hz, CF₂). UPLC-MS (Basic 4 min): rt=1.35 min, m/z (ES+) 402.2 [M+H]⁺.

### N-(2-(3,3,3-Trifluoropropoxy)pyrimidin-5-yl)-5,6-dihydrobenzo[f]imidazo[1,5-d][1,4]oxazepine-10-carboxamide 104A

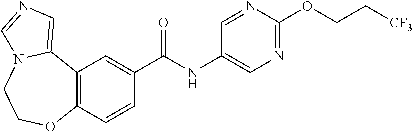 [SMILES: O=C(Nc1cnc(OCCC(F)(F)F)nc1)c1ccc2c(c1)-c1cncn1CCO2]

Prepared from lithium 5,6-dihydrobenzo[f]imidazo[1,5-d][1,4]oxazepine-10-carboxylate 4 and 2-(3,3,3-trifluoropropoxy)pyrimidin-5-amine according to General Procedure E to afford the desired product N-(2-(3,3,3-trifluoropropoxy)pyrimidin-5-yl)-5,6-dihydrobenzo[f]imidazo[1,5-d][1,4]oxazepine-10-carboxamide (29 mg, 0.0687 mmol, 24% yield) as a white solid. ¹H NMR (400 MHz, DMSO-D6) δ 10.46 (s, 1H), 8.94 (d, J=2.7 Hz, 2H), 8.42 (d, J=2.3 Hz, 1H), 7.80 (t, J=1.3 Hz, 1H), 7.78 (dd, J=8.6, 2.3 Hz, 1H), 7.66 (d, J=1.2 Hz, 1H), 7.15 (d, J=8.6 Hz, 1H), 4.58-4.37 (m, 6H), 2.84 (qt, J=11.5, 6.0 Hz, 2H). ¹⁹F NMR (376 MHz, DMSO-D6) δ-62.97 (t, J=11.4 Hz, CF₃). UPLC-MS (Basic 4 min): rt=1.45 min, m/z (ES+) 420.2 [M+H]⁺.

### N-((1r,4r)-4-((3,3-Difluoroazetidin-1-yl)methyl)cyclohexyl)-5,6-dihydrobenzo[f]imidazo[1,5-d][1,4]oxazepine-10-carboxamide 105A

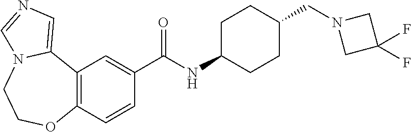 [SMILES: O=C(N[C@H]1CC[C@H](CN2CC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and (1r,4r)-4-((3,3-difluoroazetidin-1-yl)methyl)cyclohexan-1-amine di-2,2,2-trifluouroacetate according to General Procedure E to afford the desired product N-((1r,4r)-4-((3,3-difluoroazetidin-1-yl)methyl)cyclohexyl)-5,6-dihydrobenzo[f]imidazo[1,5-d][1,4]oxazepine-10-carboxamide (33 mg, 0.0788 mmol, 19% yield) as a white solid. ¹H NMR (400 MHz, DMSO-D6) δ 8.23 (d, J=2.2 Hz, 1H), 8.20 (d, J=7.8 Hz, 1H), 7.77 (d, J=1.1 Hz, 1H), 7.65 (dd, J=8.6, 2.2 Hz, 1H), 7.61 (d, J=1.1 Hz, 1H), 7.03 (d, J=8.5 Hz, 1H), 4.51-4.39 (m, 4H), 3.72 (dt, J=7.9, 3.9 Hz, 1H), 3.54 (t, J=12.5 Hz, 4H), 2.38 (d, J=6.8 Hz, 2H), 1.91-1.72 (m, 4H), 1.39-1.16 (m, 3H), 1.05-0.91 (m, 2H). ¹⁹F NMR (376 MHz, DMSO-D6) 8-97.19-−97.90 (m, CF₂). UPLC-MS (Basic 4 min): rt=1.46 min, m/z (ES+) 417.5 [M+H]⁺.

### N-((1r,4r)-4-((2-(Methylsulfonyl)ethyl)amino)cyclohexyl)-5,6-dihydrobenzo[f]imidazo[1,5-d][1,4]oxazepine-10-carboxamide 106A

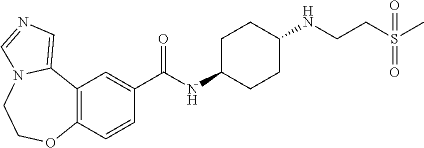 [SMILES: CS(=O)(=O)CCN[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and (1r,4r)-N1-(2-(methylsulfonyl)ethyl)cyclohexane-1,4-diamine according to General Procedure E to afford the desired product N-((1r,4r)-4-((2-(methylsulfonyl)ethyl)amino)cyclohexyl)-5,6-dihydrobenzo[f]imidazo[1,5-d][1,4]oxazepine-10-carboxamide (11 mg, 0.0259 mmol, 9% yield) as a white solid. ¹H NMR (400 MHz, DMSO-D6) δ 8.21-8.12 (m, 2H), 7.72 (d, J=1.1 Hz, 1H), 7.64-7.54 (m, 2H), 6.99 (d, J=8.6 Hz, 1H), 4.46-4.34 (m, 4H), 3.76-3.72 (m, 1H), 3.14 (t, J=6.7 Hz, 2H), 2.97 (s, 3H), 2.95-2.9 (m, 2H), 2.37-2.32 (m, 1H), 1.83-1.80 (m, 5H), 1.40-1.36 (m, 2H), 1.12-1.06 (m, 2H). UPLC-MS (Basic 4 min): rt=1.04 min, m/z (ES+) 433.3 [M−H]+

### N-((1r,4r)-4-(3-(Trifluoromethyl)azetidin-1-yl)cyclohexyl)-5,6-dihydrobenzo[f]imidazo[1,5-d][1,4]oxazepine-10-carboxamide 107A

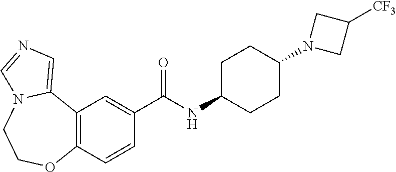 [SMILES: O=C(N[C@H]1CC[C@H](N2CC(C(F)(F)F)C2)CC1)c1ccc2c(c1)-c1cncn1CCO2]

Prepared from lithium 5,6-dihydrobenzo[f]imidazo[1,5-d][1,4]oxazepine-10-carboxylate 4 and trans-4-[3-(trifluoromethyl)azetidin-1-yl]cyclohexanamine according to General Procedure E to afford the desired product N-((1r,4r)-4-(3-(trifluoromethyl)azetidin-1-yl)cyclohexyl)-5,6-dihydrobenzo[f]imidazo[1,5-d][1,4]oxazepine-10-carboxamide (89 mg, 0.203 mmol, 48% yield) as an off-white solid. ¹H NMR (400 MHz, DMSO-D6) δ 8.21-8.12 (m, 2H), 7.72 (d, J=1.1 Hz, 1H), 7.64-7.53 (m, 2H), 6.99 (d, J=8.6 Hz, 1H), 4.46-4.35 (m, 4H), 3.73-3.61 (m, 1H), 3.35 (t, J=7.5 Hz, 2H), 3.24 (s, OH), 3.08 (t, J=6.7 Hz, 2H), 1.95 (s, 1H), 1.85-1.65 (m, 4H), 1.33-1.25 (m, 2H), 0.96-0.88 (m, 2H). ¹⁹F NMR (376 MHz, DMSO-D6) 8-70.02 (d, J=9.2 Hz, CF₃). UPLC-MS (Basic 4 min): rt=1.46 min, m/z (ES+) 465.4 [M−H]⁺.

### (R)—N-(3,3-Difluorocyclopentyl)-8-fluoro-5,6-dihydrobenzo[f]imidazo[1,5-d][1,4]oxazepine-10-carboxamide 108A

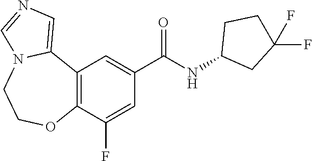 [SMILES: O=C(N[C@@H]1CCC(F)(F)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and (1R)-3,3-difluorocyclopentan-1-amine hydrochloride according to General Procedure E to afford the desired product (R)—N-(3,3-difluorocyclopentyl)-8-fluoro-5,6-dihydrobenzo[f]imidazo[1,5-d][1,4]oxazepine-10-carboxamide (103 mg, 0.290 mmol, 78% yield) as a white solid. ¹H NMR (400 MHz, DMSO-D6) δ 8.58 (d, J=7.1 Hz, 1H), 8.08 (t, J=1.8 Hz, 1H), 7.83 (d, J=1.0 Hz, 1H), 7.65 (d, J=1.1 Hz, 1H), 7.59 (dd, J=11.4, 2.1 Hz, 1H), 4.54 (s, 4H), 4.42 (tt, J=7.9, 7.9 Hz, 1H), 2.61-2.51 (m, 1H), 2.36-2.00 (m, 4H), 1.90-1.79 (m, 1H). ¹⁹F NMR (376 MHz, DMSO-D6) δ−87.52-−88.03 (m, CF₂), −130.73 (d, J=10.7 Hz, 1F). UPLC-MS (Basic 4 min): rt=1.38 min, m/z (ES+) 352.3 [M+H]⁺.

### N-(2-(2,2,2-Trifluoroethoxy)pyrimidin-5-yl)-5,6-dihydrobenzo[f]imidazo[1,5-d][1,4]oxazepine-10-carboxamide 109A

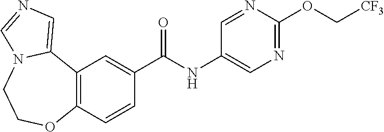 [SMILES: O=C(Nc1cnc(OCC(F)(F)F)nc1)c1ccc2c(c1)-c1cncn1CCO2]

Prepared from lithium 5,6-dihydrobenzo[f]imidazo[1,5-d][1,4]oxazepine-10-carboxylate 4 and 2-(2,2,2-trifluoroethoxy)pyrimidin-5-amine according to General Procedure E to afford the desired product N-(2-(2,2,2-trifluoroethoxy)pyrimidin-5-yl)-5,6-dihydrobenzo[f]imidazo[1,5-d][1,4]oxazepine-10-carboxamide (49 mg, 0.121 mmol, 29% yield) as a white solid. ¹H NMR (400 MHz, DMSO-D6) δ 10.52 (s, 1H), 8.99 (s, 2H), 8.43 (d, J=2.3 Hz, 1H), 7.66 (d, J=1.1 Hz, 1H), 7.15 (d, J=8.6 Hz, 1H), 5.05 (q, J=9.0 Hz, 2H), 4.55-4.44 (m, 4H). ¹⁹F NMR (376 MHz, DMSO-D6) δ-72.41 (t, J=9.0 Hz, CF₃). UPLC-MS (Basic 6 min): rt=2.43 min, m/z (ES+) 406.3 [M+H]⁺.

### N-((1r,4r)-4-((3,3,3-Trifluoropropyl)amino)cyclohexyl)-5,6-dihydrobenzo[f]imidazo[1,5-d][1,4]oxazepine-10-carboxamide 110A

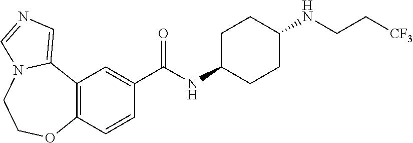 [SMILES: O=C(N[C@H]1CC[C@H](NCCC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and N1-(3,3,3-trifluoropropyl)cyclohexane-1,4-diamine according to General Procedure E to afford the desired product N-((1r,4r)-4-((3,3,3-trifluoropropyl)amino)cyclohexyl)-5,6-dihydrobenzo[f]imidazo[1,5-d][1,4]oxazepine-10-carboxamide (9 mg, 0.021 mmol, 5% yield) as a white solid. ¹H NMR (400 MHz, DMSO-D6) δ 8.23 (d, J=2.2 Hz, 1H), 8.19 (d, J=7.8 Hz, 1H), 7.77 (d, J=1.1 Hz, 1H), 7.66 (dd, J=8.6, 2.2 Hz, 1H), 7.61 (d, J=1.1 Hz, 1H), 7.03 (d, J=8.6 Hz, 1H), 4.51-4.39 (m, 4H), 3.73 (dtt, J=11.7, 7.9, 4.0 Hz, 1H), 2.75 (t, J=7.5 Hz, 2H), 2.45-2.30 (m, 3H), 1.98-1.78 (m, 4H), 1.44-1.30 (m, 2H), 1.13-1.04 (m, 2H). 1H not observed. ¹⁹F NMR (376 MHz, DMSO-D6) δ-63.46 (t, J=11.6 Hz, CF₃). UPLC-MS (Basic 6 min): rt=2.29 min, m/z (ES+) 423.5 [M+H]⁺.

### N-((1s,4s)-4-((3,3,3-Trifluoropropyl)amino)cyclohexyl)-5,6-dihydrobenzo[f]imidazo[1,5-d][1,4]oxazepine-10-carboxamide 111A

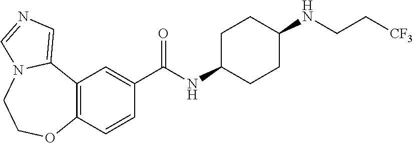 [SMILES: O=C(N[C@H]1CC[C@@H](NCCC(F)(F)F)CC1)c1ccc2c(c1)-c1cncn1CCO2]

Also isolated from the same reaction mixture was the other desired product N-((1s,4s)-4-((3,3,3-trifluoropropyl)amino)cyclohexyl)-5,6-dihydrobenzo[f]imidazo[1,5-d][1,4]oxazepine-10-carboxamide (34 mg, 0.0804 mmol, 19% yield) as a white solid. ¹H NMR (400 MHz, DMSO-D6) δ 8.26 (d, J=2.2 Hz, 1H), 8.18 (d, J=7.5 Hz, 1H), 7.77 (d, J=1.1 Hz, 1H), 7.68 (dd, J=8.6, 2.2 Hz, 1H), 7.62 (d, J=1.1 Hz, 1H), 7.03 (d, J=8.6 Hz, 1H), 4.51-4.39 (m, 4H), 3.83 (d, J=9.8 Hz, 1H), 2.76-2.66 (m, 3H), 2.47-2.31 (m, 2H), 1.92-1.57 (m, 5H), 1.57-1.41 (m, 4H). ¹⁹F NMR (376 MHz, DMSO-D6) δ-63.47 (d, J=23.3 Hz, CF₃). UPLC-MS (Basic 6 min): rt=2.38 min, m/z (ES+) 423.4 [M+H]⁺.

### (R)-8-Chloro-N-(3,3-difluorocyclopentyl)-5,6-dihydrobenzo[f]imidazo[1,5-d][1,4]oxazepine-10-carboxamide 112A

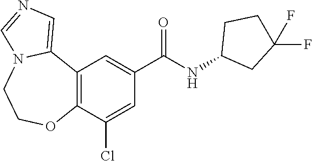 [SMILES: O=C(N[C@@H]1CCC(F)(F)C1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and (1R)-3,3-difluorocyclopentan-1-amine hydrochloride according to General Procedure E to afford the desired product (R)-8-chloro-N-(3,3-difluorocyclopentyl)-5,6-dihydrobenzo[f]imidazo[1,5-d][1,4]oxazepine-10-carboxamide (49 mg, 0.132 mmol, 68% yield) as a white solid. ¹H NMR (400 MHz, DMSO-D6) δ 8.63 (d, J=7.1 Hz, 1H), 8.20 (d, J=2.1 Hz, 1H), 7.87-7.81 (m, 2H), 7.63 (s, 1H), 4.59-4.48 (m, 4H), 4.48-4.38 (m, 1H), 2.62-2.53 (m, 1H), 2.37-2.02 (m, 4H), 1.90-1.78 (m, 1H). ¹⁹F NMR (376 MHz, DMSO-D6) δ-87.71-88.01 (m, CF₂). UPLC-MS (Basic 4 min): rt=1.49 min, m/z (ES+) 368.3, 370.3 [M+H]⁺.

### N-((1r,4r)-4-(3-(Difluoromethyl)azetidin-1-yl)cyclohexyl)-5,6-dihydrobenzo[f]imidazo[1,5-d][1,4]oxazepine-10-carboxamide 113A

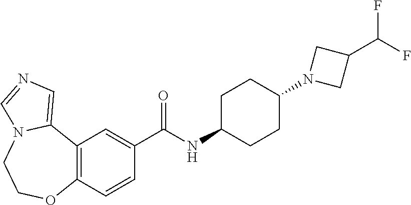 [SMILES: O=C(N[C@H]1CC[C@H](N2CC(C(F)F)C2)CC1)c1ccc2c(c1)-c1cncn1CCO2]

Prepared from lithium 5,6-dihydrobenzo[f]imidazo[1,5-d][1,4]oxazepine-10-carboxylate 4 and (1r,4r)-4-(3-(difluoromethyl)azetidin-1-yl)cyclohexan-1-amine according to General Procedure E to afford the desired product N-((1r,4r)-4-(3-(difluoromethyl)azetidin-1-yl)cyclohexyl)-5,6-dihydrobenzo[f]imidazo[1,5-d][1,4]oxazepine-10-carboxamide (38 mg, 0.0906 mmol, 21% yield) as a white solid. ¹H NMR (400 MHz, DMSO-D6) δ 8.21-8.12 (m, 2H), 7.73 (d, J=1.1 Hz, 1H), 7.64-7.54 (m, 2H), 6.99 (d, J=8.6 Hz, 1H), 6.31-6.02 (m, 1H), 4.46-4.34 (m, 4H), 3.73-3.66 (m, 1H), 3.20 (t, J=7.5 Hz, 2H), 2.99 (t, J=6.7 Hz, 2H), 2.77-2.72 (m, 1H), 1.94-1.90 (m, 1H), 1.79-1.71 (m, 4H), 1.35-1.26 (m, 2H), 1.02-0.85 (m, 2H). 19F NMR (376 MHz, DMSO-D6) δ-120.02 (dd, J=56.9, 13.8 Hz, CF₂). UPLC-MS (Basic 4 min): rt=1.33 min, m/z (ES+) 417.4 [M+H]⁺.

### 8-Fluoro-N-((1r,4r)-4-(2-hydroxypropan-2-yl)cyclohexyl)-5,6-dihydrobenzo[f]imidazo[1,5-d][1,4]oxazepine-10-carboxamide 114A

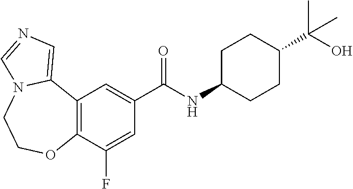 [SMILES: CC(C)(O)[C@H]1CC[C@H](NC(=O)c2cc(F)c3c(c2)-c2cncn2CCO3)CC1]

Prepared from lithium 8-fluoro-5,6-dihydrobenzo[f]imidazo[1,5-d][1,4]oxazepine-10-carboxylate and 2-(4-aminocyclohexyl) propan-2-ol according to General Procedure E to afford the desired product 8-fluoro-N-((1r,4r)-4-(2-hydroxypropan-2-yl)cyclohexyl)-5,6-dihydrobenzo[f]imidazo[1,5-d][1,4]oxazepine-10-carboxamide (37 mg, 0.0945 mmol, 24% yield) as a white solid. ¹H NMR (400 MHz, DMSO-D6) δ 8.21 (d, J=7.9 Hz, 1H), 8.03 (t, J=1.8 Hz, 1H), 7.77 (d, J=1.0 Hz, 1H), 7.61 (d, J=1.1 Hz, 1H), 7.53 (dd, J=11.5, 2.1 Hz, 1H), 4.48 (s, 4H), 4.02 (s, 1H), 3.68-3.64 (m, 1H), 1.92-1.81 (m, 4H), 1.34-1.11 (m, 5H), 1.00 (s, 6H). ¹⁹F NMR (376 MHz, DMSO-D6) δ-129.81-−132.76 (m, F). UPLC-MS (Basic 4 min): rt=1.30 min, m/z (ES+) 388.3 [M+H]⁺.

### 8-Fluoro-N-((1r,4r)-4-((2,2,2-trifluoroethyl)amino)cyclohexyl)-5,6-dihydrobenzo[f]imidazo[1,5-d][1,4]oxazepine-10-carboxamide 115A

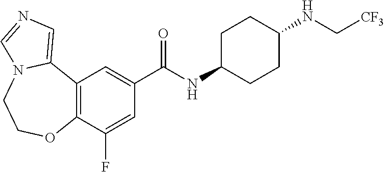 [SMILES: O=C(N[C@H]1CC[C@H](NCC(F)(F)F)CC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and trans-N4-(2,2,2-trifluoroethyl)cyclohexane-1,4-diamine according to General Procedure E to afford the desired product 8-fluoro-N-((1r,4r)-4-((2,2,2-trifluoroethyl)amino)cyclohexyl)-5,6-dihydrobenzo[f]imidazo[1,5-d][1,4]oxazepine-10-carboxamide (68 mg, 0.159 mmol, 41% yield) as an off-white solid. ¹H NMR (400 MHz, DMSO-D6) δ 8.20 (d, J=7.8 Hz, 1H), 8.02 (t, J=1.7 Hz, 1H), 7.77 (d, J=1.1 Hz, 1H), 7.60 (d, J=1.1 Hz, 1H), 7.53 (dd, J=11.5, 2.1 Hz, 1H), 4.48 (s, 4H), 3.74-3.62 (m, 1H), 3.22-3.20 (m, 1H), 2.39-2.36 (m, 1H), 2.20-2.16 (m, 1H), 1.90-1.86 (m, 2H), 1.83-1.78 (m, 2H), 1.35-1.31 (m, 2H), 1.10-1.01 (m, 2H). 1H not observed. ¹⁹F NMR (376 MHz, DMSO-D6) δ-70.58 (dd, J=10.3 Hz, CF₃). −128.57-−134.28 (m, F). UPLC-MS (Basic 4 min): rt=1.42 min, m/z (ES+) 427.4

### (R)—N-(3,3-Difluorocyclohexyl)-5,6-dihydrobenzo[f]imidazo[1,5-d][1,4]oxazepine-10-carboxamide 116A

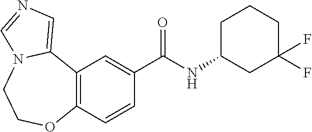 [SMILES: O=C(N[C@@H]1CCCC(F)(F)C1)c1ccc2c(c1)-c1cncn1CCO2]

Isolated by chiral separation of N-(3,3-difluorocyclohexyl)-5,6-dihydrobenzo[f]imidazo[1,5-d][1,4]oxazepine-10-carboxamide according to Purification Method B to afford the desired product (R)—N-(3,3-difluorocyclohexyl)-5,6-dihydrobenzo[f]imidazo[1,5-d][1,4]oxazepine-10-carboxamide (85.7 mg, 0.243 mmol, 32% yield) as a white solid. ¹H NMR (400 MHz, DMSO-D6) δ 8.34 (d, J=7.8 Hz, 1H), 8.24 (d, J=2.2 Hz, 1H), 7.77 (d, J=1.1 Hz, 1H), 7.66 (dd, J=8.6, 2.2 Hz, 1H), 7.59 (d, J=1.2 Hz, 1H), 7.06 (d, J=8.6 Hz, 1H), 4.50-4.47 (m, 2H), 4.46-4.40 (m, 2H), 4.12-3.93 (m, 1H), 2.36-2.22 (m, 1H), 2.10-1.63 (m, 5H), 1.54-1.32 (m, 2H). ¹⁹F NMR (376 MHz, DMSO-D6) δ-85.87 (d, J=237.6 Hz, 1F), −97.80 (dt, J=238.4, 35.5 Hz, 1F). UPLC-MS (Basic 4 min): rt=1.40 min, m/z (ES+) 348.2 [M+H]⁺.

### (S)—N-(3,3-Difluorocyclohexyl)-5,6-dihydrobenzo[f]imidazo[1,5-d][1,4]oxazepine-10-carboxamide 117A

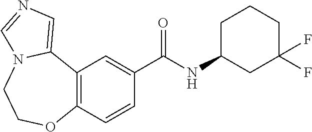 [SMILES: O=C(N[C@H]1CCCC(F)(F)C1)c1ccc2c(c1)-c1cncn1CCO2]

Isolated by chiral separation of N-(3,3-difluorocyclohexyl)-5,6-dihydrobenzo[f]imidazo[1,5-d][1,4]oxazepine-10-carboxamide according to Purification Method B to afford the desired product(S)—N-(3,3-difluorocyclohexyl)-5,6-dihydrobenzo[f]imidazo[1,5-d][1,4]oxazepine-10-carboxamide (91 mg, 0.262 mmol, 34% yield) as a white solid. ¹H NMR (400 MHz, DMSO-D6) δ 8.35 (d, J=7.8 Hz, 1H), 8.24 (d, J=2.2 Hz, 1H), 7.77 (d, J=1.1 Hz, 1H), 7.66 (dd, J=8.6, 2.2 Hz, 1H), 7.59 (d, J=1.1 Hz, 1H), 7.06 (d, J=8.6 Hz, 1H), 4.51-4.46 (m, 2H), 4.46-4.39 (m, 2H), 4.09-3.94 (m, 1H), 2.36-2.22 (m, 1H), 2.12-1.61 (m, 5H), 1.55-1.32 (m, 2H). ¹⁹F NMR (376 MHz, DMSO-D6) δ-85.87 (d, J=237.6 Hz, 1F), −97.80 (dt, J=235.7, 35.2 Hz, 1F). UPLC-MS (Basic 4 min): rt=1.40 min, m/z (ES+) 348.2 [M+H]⁺.

### N-((1s,4s)-4-((1-(Trifluoromethyl)cyclopropyl)methoxy)cyclohexyl)-5,6-dihydrobenzo[f]imidazo[1,5-d][1,4]oxazepine-10-carboxamide 118A

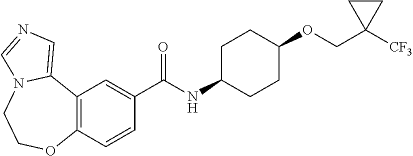 [SMILES: O=C(N[C@H]1CC[C@@H](OCC2(C(F)(F)F)CC2)CC1)c1ccc2c(c1)-c1cncn1CCO2]

Prepared from lithium 5,6-dihydrobenzo[f]imidazo[1,5-d][1,4]oxazepine-10-carboxylate 4 and 4-((1-(trifluoromethyl)cyclopropyl)methoxy)cyclohexan-1-amine according to General Procedure E to afford the desired product N-((1s,4s)-4-((1-(trifluoromethyl)cyclopropyl)methoxy)cyclohexyl)-5,6-dihydrobenzo[f]imidazo[1,5-d][1,4]oxazepine-10-carboxamide (37 mg, 0.0808 mmol, 11% yield) as a white solid. ¹H NMR (400 MHz, DMSO-D6) δ 8.32-8.17 (m, 2H), 7.77 (d, J=1.1 Hz, 1H), 7.68 (dd, J=8.6, 2.2 Hz, 1H), 7.62 (d, J=1.1 Hz, 1H), 7.03 (d, J=8.6 Hz, 1H), 4.51-4.39 (m, 4H), 3.89-3.74 (m, 1H), 3.57-3.46 (m, 3H), 1.90-1.82 (m, 2H), 1.68-1.54 (m, 4H), 1.52-1.42 (m, 2H), 1.01-0.93 (m, 2H), 0.92-0.78 (m, 2H). ¹⁹F NMR (376 MHz, DMSO-D6) δ-67.55 (s, CF₃). UPLC-MS (Basic 6 min): rt=3.03 min, m/z (ES+) 450.4 [M+H]⁺.

### N-(4-(2,2,2-Trifluoroethyl)cyclohexyl)-5,6-dihydrobenzo[f]imidazo[1,5-d][1,4]oxazepine-10-carboxamide 119A

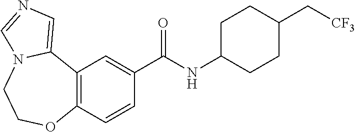 [SMILES: O=C(NC1CCC(CC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and 4-(2,2,2-trifluoroethyl)cyclohexanamine according to General Procedure E to afford the desired product N-(4-(2,2,2-trifluoroethyl)cyclohexyl)-5,6-dihydrobenzo[f]imidazo[1,5-d][1,4]oxazepine-10-carboxamide (80 mg, 0.203 mmol, 27% yield) as a white solid. ¹H NMR (400 MHz, DMSO-D6) δ 8.28-8.00 (m, 2H), 7.81-7.74 (m, 1H), 7.68-7.62 (m, 1H), 7.62-7.57 (m, 1H), 7.08-7.00 (m, 1H), 4.51-4.39 (m, 4H), 3.97-3.66 (m, 1H), 2.37-2.13 (m, 2H), 1.96-1.76 (m, 2H), 1.73-1.07 (m, 7H). ¹⁹F NMR (376 MHz, DMSO-D6) δ-61.54 (t, J=12.2 Hz, CF₃, cis), −61.98 (t, J=12.2 Hz, CF₃, trans). UPLC-MS (Basic 6 min): rt=2.82, 2.84 min, m/z (ES+) 394.4 [M+H]⁺.

### N-((1r,4r)-4-(3,3,3-Trifluoro-2,2-dimethylpropoxy)cyclohexyl)-5,6-dihydrobenzo[f]imidazo[1,5-d][1,4]oxazepine-10-carboxamide 120A

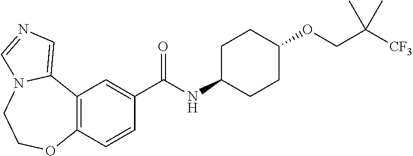 [SMILES: CC(C)(CO[C@H]1CC[C@H](NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and 4-(3,3,3-trifluoro-2,2-dimethylpropoxy)cyclohexan-1-amine according to General Procedure E to afford the desired product N-((1r,4r)-4-(3,3,3-trifluoro-2,2-dimethylpropoxy)cyclohexyl)-5,6-dihydrobenzo[f]imidazo[1,5-d][1,4]oxazepine-10-carboxamide (21 mg, 0.0446 mmol, 6% yield) as a white solid. ¹H NMR (400 MHz, DMSO-D6) δ 8.23 (d, J=2.2 Hz, 1H), 8.18 (d, J=7.7 Hz, 1H), 7.77 (d, J=1.1 Hz, 1H), 7.65 (dd, J=8.6, 2.2 Hz, 1H), 7.60 (d, J=1.2 Hz, 1H), 7.04 (d, J=8.6 Hz, 1H), 4.51-4.39 (m, 4H), 3.84-3.68 (m, 1H), 3.42 (s, 2H), 3.28-3.19 (m, 1H), 2.08-1.96 (m, 2H), 1.92-1.81 (m, 2H), 1.45-1.33 (m, 2H), 1.45-1.33 (m, 2H), 1.32-1.18 (m, 2H), 1.09 (s, 6H). ¹⁹F NMR (376 MHz, DMSO-D6) δ-75.04 (s, CF₃). UPLC-MS (Basic 6 min): rt=3.27 min, m/z (ES+) 452.5 [M+H]⁺.

### N-((1s,4s)-4-(3,3,3-Trifluoro-2,2-dimethylpropoxy)cyclohexyl)-5,6-dihydrobenzo[f]imidazo[1,5-d][1,4]oxazepine-10-carboxamide 121A

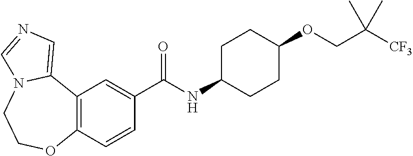 [SMILES: CC(C)(CO[C@H]1CC[C@@H](NC(=O)c2ccc3c(c2)-c2cncn2CCO3)CC1)C(F)(F)F]

Also isolated from the same reaction mixture was the other desired product N-((1s,4s)-4-(3,3,3-trifluoro-2,2-dimethylpropoxy)cyclohexyl)-5,6-dihydrobenzo[f]imidazo[1,5-d][1,4]oxazepine-10-carboxamide (67 mg, 0.148 mmol, 18% yield) as a white solid. ¹H NMR (400 MHz, DMSO-D6) δ 8.27-8.20 (m, 2H), 7.77 (d, J=1.1 Hz, 1H), 7.68 (dd, J=8.5, 2.2 Hz, 1H), 7.61 (d, J=1.1 Hz, 1H), 7.03 (d, J=8.5 Hz, 1H), 4.51-4.39 (m, 4H), 3.88-3.73 (m, 1H), 3.48 (t, J=3.1 Hz, 1H), 3.36 (s, 2H), 1.94-1.82 (m, 2H), 1.70-1.61 (m, 4H), 1.55-1.43 (m, 2H), 1.14 (s, 6H). ¹⁹F NMR (376 MHz, DMSO-D6) δ-74.99 (s, CF₃). UPLC-MS (Basic 6 min): rt=3.37 min, m/z (ES+) 452.4 [M+H]⁺.

### N-((1r,4r)-4-(2,2,2-Trifluoroethoxy)cyclohexyl)-5,6-dihydrobenzo[f]imidazo[1,5-d][1,4]oxazepine-10-carboxamide 122A

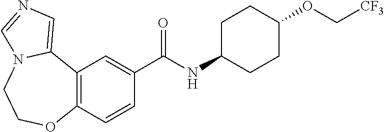 [SMILES: O=C(N[C@H]1CC[C@H](OCC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and 4-(2,2,2-trifluoroethoxy)cyclohexanamine according to General Procedure E to afford the desired product N-((1r,4r)-4-(2,2,2-trifluoroethoxy)cyclohexyl)-5,6-dihydrobenzo[f]imidazo[1,5-d][1,4]oxazepine-10-carboxamide (14 mg, 0.0337 mmol, 4% yield) as a white solid. ¹H NMR (400 MHz, DMSO-D6) δ 8.23 (d, J=2.2 Hz, 1H), 8.19 (d, J=7.8 Hz, 1H), 7.77 (d, J=1.1 Hz, 1H), 7.65 (dd, J=8.6, 2.2 Hz, 1H), 7.60 (d, J=1.1 Hz, 1H), 7.04 (d, J=8.5 Hz, 1H), 4.51-4.39 (m, 4H), 4.08 (q, J=9.4 Hz, 2H), 3.83-3.71 (m, 1H), 3.50-3.38 (m, 1H), 2.11-2.00 (m, 2H), 1.93-1.83 (m, 2H), 1.46-1.22 (m, 4H). ¹⁹F NMR (376 MHz, DMSO-D6) δ-73.07 (t, J=9.4 Hz, CF₃). UPLC-MS (Basic 6 min): rt=2.55 min, m/z (ES+) 410.4 [M+H]⁺.

### N-((1s,4s)-4-(2,2,2-Trifluoroethoxy)cyclohexyl)-5,6-dihydrobenzo[f]imidazo[1,5-d][1,4]oxazepine-10-carboxamide 123A

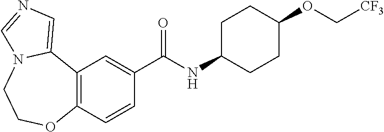 [SMILES: O=C(N[C@H]1CC[C@@H](OCC(F)(F)F)CC1)c1ccc2c(c1)-c1cncn1CCO2]

Also isolated from the same reaction mixture was the other desired product N-((1s,4s)-4-(2,2,2-trifluoroethoxy)cyclohexyl)-5,6-dihydrobenzo[f]imidazo[1,5-d][1,4]oxazepine-10-carboxamide (68 mg, 0.166 mmol, 19% yield) as a white solid. ¹H NMR (400 MHz, DMSO-D6) δ 8.33-8.19 (m, 2H), 7.77 (d, J=1.1 Hz, 1H), 7.68 (dd, J=8.5, 2.2 Hz, 1H), 7.62 (d, J=1.1 Hz, 1H), 7.03 (d, J=8.6 Hz, 1H), 4.52-4.39 (m, 4H), 4.05 (q, J=9.5 Hz, 2H), 3.91-3.82 (m, 1H), 3.70 (d, J=4.2 Hz, 1H), 1.98-1.83 (m, 2H), 1.73-1.48 (m, 6H). ¹⁹F NMR (376 MHz, DMSO-D6) δ-72.92 (t, J=9.5 Hz, CF₃). UPLC-MS (Basic 6 min): rt=2.62 min, m/z (ES+) 410.4 [M+H]⁺.

### N-(2-(3-(Difluoromethyl)azetidin-1-yl)pyrimidin-5-yl)-5,6-dihydrobenzo[f]imidazo[1,5-d][1,4]oxazepine-10-carboxamide 124A

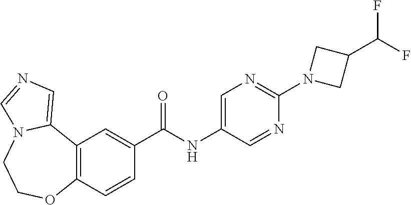 [SMILES: O=C(Nc1cnc(N2CC(C(F)F)C2)nc1)c1ccc2c(c1)-c1cncn1CCO2]

Prepared from lithium 5,6-dihydrobenzo[f]imidazo[1,5-d][1,4]oxazepine-10-carboxylate 4 and 2-[3-(difluoromethyl)azetidin-1-yl]pyrimidin-5-amine according to General Procedure E to afford the desired product N-(2-(3-(difluoromethyl)azetidin-1-yl)pyrimidin-5-yl)-5,6-dihydrobenzo[f]imidazo[1,5-d][1,4]oxazepine-10-carboxamide (38 mg, 0.0921 mmol, 22% yield) as a white solid. ¹H NMR (400 MHz, DMSO-D6) δ 10.20 (s, 1H), 8.67 (s, 2H), 8.40 (d, J=2.2 Hz, 1H), 7.79-7.69 (m, 2H), 7.65 (d, J=1.1 Hz, 1H), 7.13 (d, J=8.6 Hz, 1H), 6.48-6.39 (m, 1H), 4.52-4.39 (m, 4H), 4.15 (t, J=8.7 Hz, 2H), 3.95 (dd, J=9.0, 5.5 Hz, 2H), 3.27-3.10 (m, 1H). ¹⁹F NMR (376 MHz, DMSO-D6) δ-123.79 (dd, J=56.5, 15.0 Hz, CF₂). UPLC-MS (Basic 4 min): rt=1.27 min, m/z (ES+) 413.4 [M+H]⁺.

### N-(4-Ethylcyclohexyl)-5,6-dihydrobenzo[f]imidazo[1,5-d][1,4]oxazepine-10-carboxamide 125A

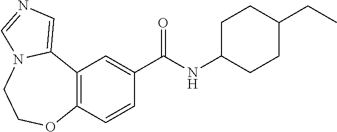 [SMILES: CCC1CCC(NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and an inseparable mixture of 4-ethylcyclohexanamine and 1-(4-aminocyclohexyl) ethanol according to General Procedure E to afford the desired product N-(4-ethylcyclohexyl)-5,6-dihydrobenzo[f]imidazo[1,5-d][1,4]oxazepine-10-carboxamide (36 mg, 0.106 mmol, 8% yield) as a white solid. ¹H NMR (400 MHz, DMSO-D6) δ 8.27-8.20 (m, 1H), 8.20-8.03 (m, 1H), 7.80-7.74 (m, 1H), 7.69-7.63 (m, 1H), 7.63-7.57 (m, 1H), 7.07-7.00 (m, 1H), 4.51-4.39 (m, 4H), 3.96-3.65 (m, 1H), 1.88-1.74 (m, 1H), 1.71-1.46 (m, 6H), 1.39-1.18 (m, 3H), 1.18-0.92 (m, 1H), 0.87 (td, J=7.3, 1.7 Hz, 3H). UPLC-MS (Basic 6 min): rt=2.94 min, m/z (ES+) 340.4 [M+H]⁺.

### N-(4-(1-Hydroxyethyl)cyclohexyl)-5,6-dihydrobenzo[f]imidazo[1,5-d][1,4]oxazepine-10-carboxamide 126A

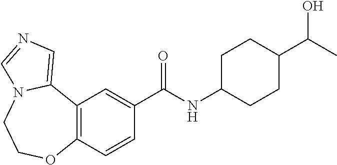 [SMILES: CC(O)C1CCC(NC(=O)c2ccc3c(c2)-c2cncn2CCO3)CC1]

Also isolated from the same reaction mixture was the other desired product N-(4-(1-hydroxyethyl)cyclohexyl)-5,6-dihydrobenzo[f]imidazo[1,5-d][1,4]oxazepine-10-carboxamide (35 mg, 0.0963 mmol, 8% yield) as a white solid. ¹H NMR (400 MHz, DMSO-D6) δ 8.25-8.19 (m, 1H), 8.19-7.96 (m, 1H), 7.77 (d, J=1.2 Hz, 1H), 7.69-7.63 (m, 1H), 7.63-7.56 (m, 1H), 7.07-7.00 (m, 1H), 4.51-4.39 (m, 4H), 4.32 (d, J=5.2 Hz, 1H), 3.99-3.64 (m, 1H), 3.63-3.33 (m, 1H), 2.00-1.60 (m, 3H), 1.60-1.38 (m, 4H), 1.37-1.09 (m, 2H), 1.09-0.97 (m, 3H). UPLC-MS (Basic 6 min): rt=1.81 min, m/z (ES+) 356.4 [M+H]⁺.

### N-((1r,4r)-4-(2-Methoxy-2-methylpropoxy)cyclohexyl)-5,6-dihydrobenzo[f]imidazo[1,5-d][1,4]oxazepine-10-carboxamide 127A

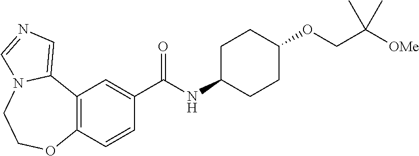 [SMILES: COC(C)(C)CO[C@H]1CC[C@H](NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and 4-(2-methoxy-2-methyl-propoxy)cyclohexanamine according to General Procedure E to afford the desired product N-((1r,4r)-4-(2-methoxy-2-methylpropoxy)cyclohexyl)-5,6-dihydrobenzo[f]imidazo[1,5-d][1,4]oxazepine-10-carboxamide (12 mg, 0.0290 mmol, 3% yield) as a white solid. ¹H NMR (400 MHz, DMSO-D6) δ 8.23 (d, J=2.2 Hz, 1H), 8.18 (d, J=7.8 Hz, 1H), 7.77 (s, 1H), 7.65 (dd, J=8.6, 2.2 Hz, 1H), 7.60 (s, 1H), 7.04 (d, J=8.5 Hz, 1H), 4.51-4.39 (m, 4H), 3.84-3.68 (m, 1H), 3.29 (s, 2H), 3.27-3.16 (m, 1H), 3.11 (s, 3H), 2.06-1.98 (m, 2H), 1.91-1.82 (m, 2H), 1.44-1.34 (m, 2H), 1.30-1.18 (m, 2H), 1.07 (s, 6H). UPLC-MS (Basic 6 min): rt=2.40 min, m/z (ES+) 414.5 [M+H]⁺.

### N-((1s,4s)-4-(2-Methoxy-2-methylpropoxy)cyclohexyl)-5,6-dihydrobenzo[f]imidazo[1,5-d][1,4]oxazepine-10-carboxamide 128A

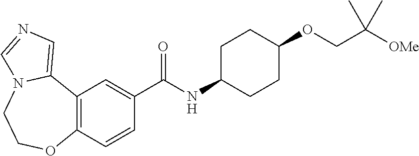 [SMILES: COC(C)(C)CO[C@H]1CC[C@@H](NC(=O)c2ccc3c(c2)-c2cncn2CCO3)CC1]

Also isolated from the same reaction mixture was the other desired product N-((1s,4s)-4-(2-methoxy-2-methylpropoxy)cyclohexyl)-5,6-dihydrobenzo[f]imidazo[1,5-d][1,4]oxazepine-10-carboxamide (93 mg, 0.221 mmol, 23% yield) as a white solid. ¹H NMR (400 MHz, DMSO-D6) δ 8.29-8.19 (m, 2H), 7.77 (d, J=1.1 Hz, 1H), 7.68 (dd, J=8.6, 2.2 Hz, 1H), 7.61 (d, J=1.1 Hz, 1H), 7.03 (d, J=8.6 Hz, 1H), 4.51-4.39 (m, 4H), 3.87-3.75 (m, 1H), 3.47 (s, 1H), 3.24 (s, 2H), 3.14 (s, 3H), 1.97-1.78 (m, 2H), 1.74-1.54 (m, 4H), 1.51-1.43 (m, 2H), 1.12 (s, 6H). UPLC-MS (Basic 6 min): rt=2.54 min, m/z (ES+) 414.5 [M+H]⁺.

### 8-Chloro-N-(2,3-dihydro-[1,4]dioxino[2,3-b]pyridin-7-yl)-5,6-dihydrobenzo[f]imidazo[1,5-d][1,4]oxazepine-10-carboxamide 129A

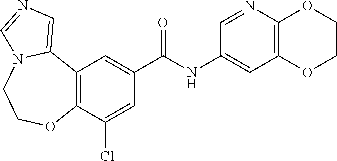 [SMILES: O=C(Nc1cnc2c(c1)OCCO2)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 2,3-dihydro-[1,4]dioxino[2,3-b]pyridin-7-amine dihydrochloride according to General Procedure E, followed by precipitation from MeCN, to afford the desired product 8-chloro-N-(2,3-dihydro-[1,4]dioxino[2,3-b]pyridin-7-yl)-5,6-dihydrobenzo[f]imidazo[1,5-d][1,4]oxazepine-10-carboxamide (5.2 mg, 0.0130 mmol, 7% yield) as a white solid. ¹H NMR (400 MHz, DMSO-D6) δ 10.33 (s, 1H), 8.32 (d, J=2.1 Hz, 1H), 8.09 (d, J=2.4 Hz, 1H), 7.94 (d, J=2.1 Hz, 1H), 7.85 (s, 1H), 7.73 (d, J=2.3 Hz, 1H), 7.69 (s, 1H), 4.63-4.51 (m, 4H), 4.42-4.36 (m, 2H), 4.30-4.23 (m, 2H). UPLC-MS (Basic 4 min): rt=1.33 min, m/z (ES+) 399.3, 401.3 [M+H]⁺.

### (R)-8-Fluoro-N-(tetrahydro-2H-pyran-3-yl)-5,6-dihydrobenzo[f]imidazo[1,5-d][1,4]oxazepine-10-carboxamide 130A

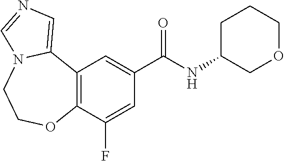 [SMILES: O=C(N[C@@H]1CCCOC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and (R)-tetrahydro-2H-pyran-3-amine hydrochloride according to General Procedure E to afford the desired product (R)-8-fluoro-N-(tetrahydro-2H-pyran-3-yl)-5,6-dihydrobenzo[f]imidazo[1,5-d][1,4]oxazepine-10-carboxamide (83 mg, 0.251 mmol, 64% yield) as a white solid. ¹H NMR (400 MHz, DMSO-D6) δ 8.22 (d, J=7.6 Hz, 1H), 8.04 (t, J=1.8 Hz, 1H), 7.78 (d, J=1.1 Hz, 1H), 7.61 (d, J=1.1 Hz, 1H), 7.55 (dd, J=11.5, 2.1 Hz, 1H), 4.49 (s, 4H), 3.94-3.88 (m, 1H), 3.85-3.69 (m, 2H), 3.27-3.24 (m, 1H), 3.12 (dd, J=10.6, 9.5 Hz, 1H), 1.95-1.92 (m, 1H), 1.72-1.47 (m, 3H). ¹⁹F NMR (376 MHz, DMSO-D6) δ-130.82 (dd, J=11.4, 1.6 Hz, F). UPLC-MS (Basic 4 min): rt=1.25 min, m/z (ES+) 320.3 [M+H]⁺.

### N-((1r,4r)-4-((2-Hydroxy-2-(trifluoromethyl)butyl)amino)cyclohexyl)-5,6-dihydrobenzo[f]imidazo[1,5-d][1,4]oxazepine-10-carboxamide 131A

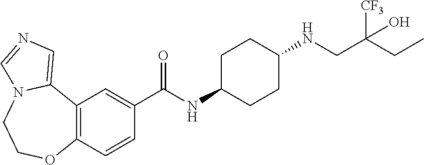 [SMILES: CCC(O)(CN[C@H]1CC[C@H](NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and 2-[[(4-aminocyclohexyl)amino]methyl]-1,1,1-trifluoro-butan-2-ol according to General Procedure E to afford the desired product N-((1r,4r)-4-((2-hydroxy-2-(trifluoromethyl)butyl)amino)cyclohexyl)-5,6-dihydrobenzo[f]imidazo[1,5-d][1,4]oxazepine-10-carboxamide (21 mg, 0.0454 mmol, 11% yield) as a white solid. ¹H NMR (400 MHz, DMSO-D6) δ 8.19 (d, J=2.2 Hz, 1H), 8.14 (d, J=7.9 Hz, 1H), 7.73 (d, J=1.1 Hz, 1H), 7.61 (dd, J=8.6, 2.2 Hz, 1H), 7.56 (d, J=1.1 Hz, 1H), 6.99 (d, J=8.6 Hz, 1H), 5.60 (s, 1H), 4.46-4.36 (m, 4H), 3.77-3.58 (m, 1H), 2.79-2.56 (m, 2H), 2.33-2.31 (m, 1H), 1.94-1.83 (m, 4H), 1.63 (q, J=7.5 Hz, 2H), 1.57-1.55 (m, 1H), 1.38-1.27 (m, 2H), 1.18-1.12 (m, 2H), 0.84 (t, J=7.5 Hz, 3H). ¹⁹F NMR (376 MHz, DMSO-D6) δ-77.53 (s, CF₃). UPLC-MS (Basic 4 min): rt=1.55 min, m z (ES+) 467.4 [M+H]⁺.

### (R)—N-(1,1-Dioxidotetrahydrothiophen-3-yl)-8-fluoro-5,6-dihydrobenzo[f]imidazo[1,5-d][1,4]oxazepine-10-carboxamide 132A

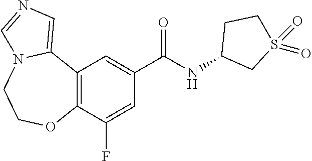 [SMILES: O=C(N[C@@H]1CCS(=O)(=O)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and (3R)-1,1-dioxothiolan-3-amine hydrochloride according to General Procedure E to afford the desired product (R)—N-(1,1-dioxidotetrahydrothiophen-3-yl)-8-fluoro-5,6-dihydrobenzo[f]imidazo[1,5-d][1,4]oxazepine-10-carboxamide (95 mg, 0.260 mmol, 66% yield) as a white solid. ¹H NMR (400 MHz, DMSO-D6) δ 8.76 (d, J=7.0 Hz, 1H), 8.09 (t, J=1.8 Hz, 1H), 7.83 (d, J=1.1 Hz, 1H), 7.64 (d, J=1.1 Hz, 1H), 7.59 (dd, J=11.3, 2.1 Hz, 1H), 4.76-4.62 (m, 1H), 4.54 (s, 4H), 3.52 (dd, J=13.5, 8.1 Hz, 1H), 3.44-3.33 (m, 1H), 3.27-3.15 (m, 1H), 3.07 (ddd, J=13.5, 8.0, 1.2 Hz, 1H), 2.50-2.39 (m, 1H), 2.30-2.15 (m, 1H). ¹⁹F NMR (376 MHz, DMSO-D6) δ-130.58 (dd, J=11.4, 1.5 Hz, F). UPLC-MS (Basic 4 min): rt=1.01 min, m/z (ES+) 366.3 [M+H]⁺.

### N-(4-(1,1,1-Trifluoropropan-2-yl)cyclohexyl)-5,6-dihydrobenzo[f]imidazo[1,5-d][1,4]oxazepine-10-carboxamide 133A

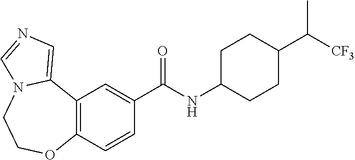 [SMILES: CC(C1CCC(NC(=O)c2ccc3c(c2)-c2cncn2CCO3)CC1)C(F)(F)F]

Prepared from lithium 5,6-dihydrobenzo[f]imidazo[1,5-d][1,4]oxazepine-10-carboxylate 4 and 4-(2,2,2-trifluoro-1-methyl-ethyl)cyclohexanamine according to General Procedure E to afford the desired product N-(4-(1,1,1-trifluoropropan-2-yl)cyclohexyl)-5,6-dihydrobenzo[f]imidazo[1,5-d][1,4]oxazepine-10-carboxamide (30 mg, 0.0736 mmol, 11% yield) as a white solid. ¹H NMR (400 MHz, DMSO-D6) δ 8.28-7.98 (m, 2H), 7.82-7.74 (m, 1H), 7.69-7.62 (m, 1H), 7.62-7.54 (m, 1H), 7.08-7.00 (m, 1H), 4.51-4.39 (m, 4H), 4.05-3.67 (m, 1H), 2.45-2.28 (m, 1H), 1.95-1.78 (m, 2H), 1.73-1.53 (m, 4H), 1.53-1.20 (m, 3H), 1.11-1.02 (m, 3H). ¹⁹F NMR (376 MHz, DMSO-D6) δ-67.64 (d, J=10.2 Hz, cis CF₃), —67.94 (d, J=10.5 Hz, trans CF₃). UPLC-MS (Basic 6 min): rt=3.02 min, m/z (ES+) 408.4

### 8-Chloro-N-(3,3-difluorocyclobutyl)-5,6-dihydrobenzo[f]imidazo[1,5-d][1,4]oxazepine-10-carboxamide 134A

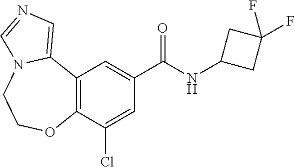 [SMILES: O=C(NC1CC(F)(F)C1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 3,3-difluorocyclobutanamine hydrochloride according to General Procedure E to afford the desired product 8-chloro-N-(3,3-difluorocyclobutyl)-5,6-dihydrobenzo[f]imidazo[1,5-d][1,4]oxazepine-10-carboxamide (40 mg, 0.108 mmol, 56% yield) as a white solid. ¹H NMR (400 MHz, DMSO-D6) δ 8.88 (d, J=6.6 Hz, 1H), 8.21 (d, J=2.2 Hz, 1H), 7.86-7.81 (m, 2H), 7.63 (d, J=1.1 Hz, 1H), 4.60-4.49 (m, 4H), 4.33-4.21 (m, 1H), 3.05-2.90 (m, 1H), 2.85-2.68 (m, 1H). ¹⁹F NMR (376 MHz, DMSO-D6) δ−81.00-−82.21 (m, F), −95.71-−97.61 (m, F). UPLC-MS (Basic 4 min): rt=1.43 min, m/z (ES+) 352.1, 354.1 [M+H]⁺.

### N-(3,3-Difluorocyclobutyl)-8-fluoro-5,6-dihydrobenzo[f]imidazo[1,5-d][1,4]oxazepine-10-carboxamide 135A

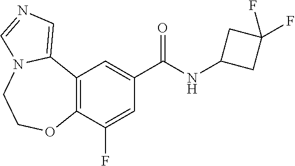 [SMILES: O=C(NC1CC(F)(F)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 3,3-difluorocyclobutanamine hydrochloride according to General Procedure E to afford the desired product N-(3,3-difluorocyclobutyl)-8-fluoro-5,6-dihydrobenzo[f]imidazo[1,5-d][1,4]oxazepine-10-carboxamide (93 mg, 0.273 mmol, 69% yield) as a white solid. ¹H NMR (400 MHz, DMSO-D6) δ 8.79 (d, J=6.5 Hz, 1H), 8.05 (t, J=1.8 Hz, 1H), 7.78 (d, J=1.0 Hz, 1H), 7.60 (d, J=1.1 Hz, 1H), 7.53 (dd, J=11.4, 2.1 Hz, 1H), 4.50 (s, 4H), 4.31-4.04 (m, 1H), 3.00-2.80 (m, 2H), 2.80-2.64 (m, 2H). ¹⁹F NMR (376 MHz, DMSO-D6) δ-81.64 (dd, J=195.3, 5.3 Hz, F), −95.25--98.53 (m, F), −130.60 (dd, J=11.3, 1.6 Hz, F). UPLC-MS (Basic 4 min): rt=1.32 min, m/z (ES+) 338.3 [M+H]⁺.

### N-(3-Fluoro-3-methylcyclobutyl)-5,6-dihydrobenzo[f]imidazo[1,5-d][1,4]oxazepine-10-carboxamide 136A

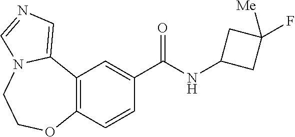 [SMILES: CC1(F)CC(NC(=O)c2ccc3c(c2)-c2cncn2CCO3)C1]

Prepared from lithium 5,6-dihydrobenzo[f]imidazo[1,5-d][1,4]oxazepine-10-carboxylate 4 and 3-fluoro-3-methylcyclobutanamine hydrochloride according to General Procedure E to afford the desired product N-(3-fluoro-3-methylcyclobutyl)-5,6-dihydrobenzo[f]imidazo[1,5-d][1,4]oxazepine-10-carboxamide (55 mg, 0.169 mmol, 40% yield) as a white solid. ¹H NMR (400 MHz, DMSO-D6) δ 8.58 (d, J=6.7 Hz, 1H), 8.20 (d, J=2.2 Hz, 1H), 7.73 (d, J=1.1 Hz, 1H), 7.61 (dd, J=8.5, 2.2 Hz, 1H), 7.56 (d, J=1.1 Hz, 1H), 7.01 (d, J=8.6 Hz, 1H), 4.56-4.28 (m, 5H), 2.65-2.50 (m, 2H), 2.34-2.12 (m, 2H), 1.44 (d, J=22.2 Hz, 3H). ¹⁹F NMR (376 MHz, DMSO-D6) δ−136.38-−139.99 (m, F). UPLC-MS (Basic 4 min): rt=1.25 min, m/z (ES+) 316.1 [M+H]⁺.

### 8-Chloro-N-(4,4-difluorocyclohexyl)-5,6-dihydrobenzo[f]imidazo[1,5-d][1,4]oxazepine-10-carboxamide 137A

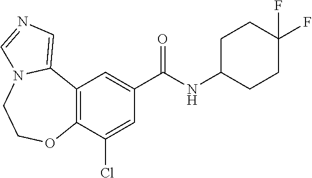 [SMILES: O=C(NC1CCC(F)(F)CC1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 4,4-difluorocyclohexanamine hydrochloride according to General Procedure E to afford the desired product 8-chloro-N-(4,4-difluorocyclohexyl)-5,6-dihydrobenzo[f]imidazo[1,5-d][1,4]oxazepine-10-carboxamide (20 mg, 0.0523 mmol, 27% yield) as a white solid. ¹H NMR (400 MHz, DMSO-D6) δ 8.37 (d, J=7.7 Hz, 1H), 8.19 (d, J=2.1 Hz, 1H), 7.84 (dd, J=3.7, 1.6 Hz, 2H), 7.63 (d, J=1.1 Hz, 1H), 4.59-4.48 (m, 4H), 4.03-3.96 (m, 1H), 2.14-1.84 (m, 6H), 1.72-1.57 (m, 2H). ¹⁹F NMR (376 MHz, DMSO-D6) δ−90.78-−93.03 (m, F), −98.50-−99.68 (m, F). UPLC-MS (Basic 4 min): rt=1.54 min, m/z (ES+) 382.3, 384.3 [M+H]⁺.

### 8-Fluoro-N-(3-fluoro-3-methylcyclobutyl)-5,6-dihydrobenzo[f]imidazo[1,5-d][1,4]oxazepine-10-carboxamide 138A

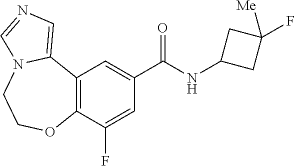 [SMILES: CC1(F)CC(NC(=O)c2cc(F)c3c(c2)-c2cncn2CCO3)C1]

Prepared from lithium 8-fluoro-5,6-dihydrobenzo[f]imidazo[1,5-d][1,4]oxazepine-10-carboxylate and 3-fluoro-3-methylcyclobutanamine hydrochloride according to General Procedure E to afford the desired product 8-fluoro-N-(3-fluoro-3-methylcyclobutyl)-5,6-dihydrobenzo[f]imidazo[1,5-d][1,4]oxazepine-10-carboxamide (37 mg, 0.110 mmol, 28% yield) as a white solid. ¹H NMR (400 MHz, DMSO-D6) δ 8.64 (d, J=6.6 Hz, 1H), 8.02 (t, J=1.9 Hz, 1H), 7.78 (d, J=1.1 Hz, 1H), 7.59 (d, J=1.1 Hz, 1H), 7.53 (dd, J=11.4, 2.1 Hz, 1H), 4.49 (s, 4H), 4.50-4.37 (m, 1H), 2.60-2.55 (m, 2H), 2.33-2.15 (m, 2H), 1.44 (d, J=22.2 Hz, 3H). 19F NMR (376 MHz, DMSO-D6) δ−130.75 (dd, J=11.5, 1.6 Hz, F), −136.51-−139.63 (m, F). UPLC-MS (Basic 4 min): rt=1.32 min, m/z (ES+) 334.1 [M+H]⁺.

### N-(2,2-Difluorocyclobutyl)-5,6-dihydrobenzo[f]imidazo[1,5-d][1,4]oxazepine-10-carboxamide 139A

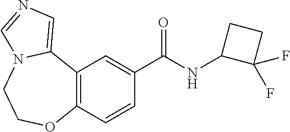 [SMILES: O=C(NC1CCC1(F)F)c1ccc2c(c1)-c1cncn1CCO2]

Prepared from lithium 5,6-dihydrobenzo[f]imidazo[1,5-d][1,4]oxazepine-10-carboxylate 4 and 2,2-difluorocyclobutan-1-amine hydrochloride according to General Procedure E to afford the desired product N-(2,2-difluorocyclobutyl)-5,6-dihydrobenzo[f]imidazo[1,5-d][1,4]oxazepine-10-carboxamide (92 mg, 0.289 mmol, 68% yield) as a white solid. ¹H NMR (400 MHz, DMSO-D6) δ 8.74 (d, J=7.7 Hz, 1H), 8.27 (d, J=2.2 Hz, 1H), 7.74 (d, J=1.1 Hz, 1H), 7.67 (dd, J=8.6, 2.2 Hz, 1H), 7.58 (d, J=1.1 Hz, 1H), 7.03 (d, J=8.6 Hz, 1H), 4.91-4.84 (m, 1H), 4.48-4.37 (m, 4H), 2.45-2.29 (m, 2H), 2.13-2.11 (m, 1H), 1.95-1.92 (m, 1H). 19F NMR (376 MHz, DMSO-D6) δ−83.55-−84.07 (m, F), −115.45-116.09 (m, F). UPLC-MS (Basic 4 min): rt=1.22 min, m/z (ES+) 320.0 [M+H]⁺.

### N-(2,2-Difluorocyclobutyl)-8-fluoro-5,6-dihydrobenzo[f]imidazo[1,5-d][1,4]oxazepine-10-carboxamide 140A

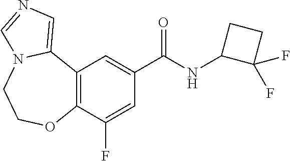 [SMILES: O=C(NC1CCC1(F)F)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2,2-difluorocyclobutan-1-amine hydrochloride according to General Procedure E to afford the desired product N-(2,2-difluorocyclobutyl)-8-fluoro-5,6-dihydrobenzo[f]imidazo[1,5-d][1,4]oxazepine-10-carboxamide (46 mg, 0.136 mmol, 35% yield) as a white solid. ¹H NMR (400 MHz, DMSO-D6) δ 8.80 (d, J=7.7 Hz, 1H), 8.10 (t, J=1.8 Hz, 1H), 7.78 (d, J=1.1 Hz, 1H), 7.66-7.54 (m, 2H), 4.92-4.76 (m, 1H), 4.50 (s, 4H), 2.45-2.29 (m, 2H), 2.15-2.12 (m, 1H), 1.98-1.91 (m, 1H). ¹⁹F NMR (376 MHz, DMSO-D6) δ−83.54-−84.06 (m, F), −115.48-116-12 (m, F), −130.53 (dd, J=11.6, 1.7 Hz, F). UPLC-MS (Basic 4 min): rt=1.29 min, m/z (ES+) 338.3 [M+H]⁺.

### N-(3,3-Dimethylcyclobutyl)-5,6-dihydrobenzo[f]imidazo[1,5-d][1,4]oxazepine-10-carboxamide 141A

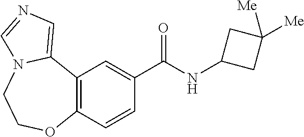 [SMILES: CC1(C)CC(NC(=O)c2ccc3c(c2)-c2cncn2CCO3)C1]

Prepared from lithium 5,6-dihydrobenzo[f]imidazo[1,5-d][1,4]oxazepine-10-carboxylate 4 and 3,3-dimethylcyclobutan-1-amine hydrochloride according to General Procedure E to afford the desired product N-(3,3-dimethylcyclobutyl)-5,6-dihydrobenzo[f]imidazo[1,5-d][1,4]oxazepine-10-carboxamide (54 mg, 0.173 mmol, 43% yield) as a white solid. ¹H NMR (400 MHz, DMSO-D6) δ 8.51 (d, J=7.4 Hz, 1H), 8.24 (d, J=2.2 Hz, 1H), 7.77 (d, J=1.1 Hz, 1H), 7.65 (dd, J=8.6, 2.2 Hz, 1H), 7.62 (d, J=1.1 Hz, 1H), 7.04 (d, J=8.6 Hz, 1H), 4.51-4.34 (m, 5H), 2.14-2.03 (m, 2H), 1.95-1.85 (m, 2H), 1.16 (s, 3H), 1.13 (s, 3H). UPLC-MS (Basic 4 min): rt=1.49 min, m/z (ES+) 312.3 [M+H]⁺.

### N-((1r,4r)-4-((1-(Trifluoromethyl)cyclopropyl)methoxy)cyclohexyl)-5,6-dihydrobenzo[f]imidazo[1,5-d][1,4]oxazepine-10-carboxamide 142A

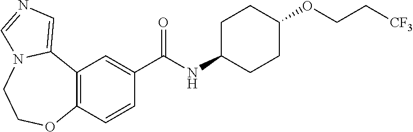 [SMILES: O=C(N[C@H]1CC[C@H](OCCC(F)(F)F)CC1)c1ccc2c(c1)-c1cncn1CCO2]

Prepared from lithium 5,6-dihydrobenzo[f]imidazo[1,5-d][1,4]oxazepine-10-carboxylate 4 and 4-((1-(trifluoromethyl)cyclopropyl)methoxy)cyclohexan-1-amine according to General Procedure E to afford the desired product N-((1r,4r)-4-((1-(trifluoromethyl)cyclopropyl)methoxy)cyclohexyl)-5,6-dihydrobenzo[f]imidazo[1,5-d][1,4]oxazepine-10-carboxamide (3.6 mg, 0.0073 mmol, 1% yield) as a white solid. ¹H NMR (400 MHz, DMSO-D6) δ 8.23 (d, J=2.2 Hz, 1H), 8.18 (d, J=7.9 Hz, 1H), 7.77 (s, 1H), 7.68-7.62 (m, 1H), 7.60 (s, 1H), 7.03 (d, J=8.6 Hz, 1H), 4.49-4.39 (m, 4H), 3.78-3.72 (m, 1H), 3.56 (s, 2H), 2.05-1.95 (m, 2H), 1.90-1.82 (m, 2H), 1.42-1.36 (m, 2H), 1.28-1.22 (m, 2H), 0.95 (d, J=6.6 Hz, 2H), 0.82 (s, 2H), 1H not observed. ¹⁹F NMR (376 MHz, DMSO-D6) 8-67.59 (s, CF₃). UPLC-MS (Basic 6 min): rt=2.90 min, m/z (ES+) 450.4 [M+H]⁺.

### N-(3,3-Dimethylcyclobutyl)-8-fluoro-5,6-dihydrobenzo[f]imidazo[1,5-d][1,4]oxazepine-10-carboxamide 143A

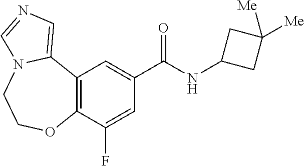 [SMILES: CC1(C)CC(NC(=O)c2cc(F)c3c(c2)-c2cncn2CCO3)C1]

Prepared from lithium 8-fluoro-5,6-dihydrobenzo[f]imidazo[1,5-d][1,4]oxazepine-10-carboxylate and 3,3-dimethylcyclobutan-1-amine hydrochloride according to General Procedure E to afford the desired product N-(3,3-dimethylcyclobutyl)-8-fluoro-5,6-dihydrobenzo[f]imidazo[1,5-d][1,4]oxazepine-10-carboxamide (41 mg, 0.124 mmol, 32% yield) as a white solid. ¹H NMR (400 MHz, DMSO-D6) δ 8.57 (d, J=7.3 Hz, 1H), 8.07 (t, J=1.8 Hz, 1H), 7.81 (d, J=1.1 Hz, 1H), 7.65 (d, J=1.1 Hz, 1H), 7.57 (dd, J=11.5, 2.1 Hz, 1H), 4.53 (s, 4H), 4.47-4.33 (m, 1H), 2.14-2.04 (m, 2H), 1.95-1.84 (m, 2H), 1.16 (s, 3H), 1.13 (s, 3H). ¹⁹F NMR (376 MHz, DMSO-D6) δ-130.85 (dd, J=11.4, 1.6 Hz, F). UPLC-MS (Basic 4 min): rt=1.55 min, m/z (ES+) 330.3 [M+H]⁺.

### N-(3-(Methoxymethyl)-2,3-dihydro-[1,4]dioxino[2,3-b]pyridin-7-yl)-5,6-dihydrobenzo[f]imidazo[1,5-d][1,4]oxazepine-10-carboxamide 144A

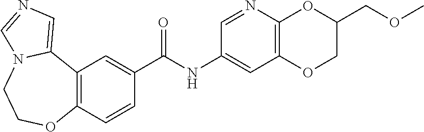 [SMILES: COCC1COc2cc(NC(=O)c3ccc4c(c3)-c3cncn3CCO4)cnc2O1]

Prepared from lithium 5,6-dihydrobenzo[f]imidazo[1,5-d][1,4]oxazepine-10-carboxylate 4 and an inseparable mixture of 3-(methoxymethyl)-2,3-dihydro-[1,4]dioxino[2,3-b]pyridin-7-amine and 2-(methoxymethyl)-2,3-dihydro-[1,4]dioxino[2,3-b]pyridin-7-amine according to General Procedure E to afford the desired product N-(3-(methoxymethyl)-2,3-dihydro-[1,4]dioxino[2,3-b]pyridin-7-yl)-5,6-dihydrobenzo[f]imidazo[1,5-d][1,4]oxazepine-10-carboxamide (12 mg, 0.0282 mmol, 15% yield) as a white solid. ¹H NMR (400 MHz, ACETONITRILE-D3) δ 10.23 (s, 1H), 8.38 (d, J=2.3 Hz, 1H), 8.12 (d, J=2.3 Hz, 1H), 7.79 (d, J=1.1 Hz, 1H), 7.77 (d, J=2.2 Hz, 1H), 7.75 (t, J=2.2 Hz, 2H), 7.65 (d, J=1.2 Hz, 1H), 7.12 (d, J=8.5 Hz, 1H), 4.57-4.50 (m, 2H), 4.50 (m, 2H), 4.49-4.44 (m, 2H), 4.36 (dd, J=11.5, 2.5 Hz, 1H), 4.04 (dd, J=11.6, 7.4 Hz, 1H), 3.63 (dd, J=5.0, 2.7 Hz, 2H), 3.34 (s, 3H). UPLC-MS (Basic 6 min): rt=1.96 min, m/z (ES+) 409.4 [M+H]⁺.

### N-(2-(Methoxymethyl)-2,3-dihydro-[1,4]dioxino[2,3-b]pyridin-7-yl)-5,6-dihydrobenzo[f]imidazo[1,5-d][1,4]oxazepine-10-carboxamide 145A

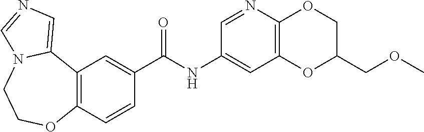 [SMILES: COCC1COc2ncc(NC(=O)c3ccc4c(c3)-c3cncn3CCO4)cc2O1]

Also isolated from the same reaction mixture was the other desired product N-(2-(methoxymethyl)-2,3-dihydro-[1,4]dioxino[2,3-b]pyridin-7-yl)-5,6-dihydrobenzo[f]imidazo[1,5-d][1,4]oxazepine-10-carboxamide (4.8 mg, 0.0118 mmol, 6% yield) as an off-white solid. 1H NMR (400 MHz, ACETONITRILE-D3) δ 8.80 (s, 1H), 8.30 (d, J=2.3 Hz, 1H), 8.05 (d, J=2.3 Hz, 1H), 7.72 (dd, J=8.6, 2.2 Hz, 1H), 7.68 (d, J=2.3 Hz, 1H), 7.59 (s, 2H), 7.10 (d, J=8.6 Hz, 1H), 4.46 (tt, J=3.2, 1.7 Hz, 4H), 4.42 (d, J=2.4 Hz, 1H), 4.37 (dtd, J=7.4, 5.0, 2.4 Hz, 1H), 4.20 (dd, J=11.6, 7.2 Hz, 1H), 3.70-3.58 (m, 2H), 3.38 (s, 3H). UPLC-MS (Basic 6 min): rt=1.93 min, m/z (ES+) 409.4 [M+H]⁺.

### N-(4-(Methylsulfonyl)cyclohexyl)-5,6-dihydrobenzo[f]imidazo[1,5-d][1,4]oxazepine-10-carboxamide 146A

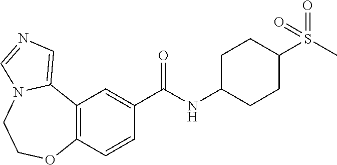 [SMILES: CS(=O)(=O)C1CCC(NC(=O)c2ccc3c(c2)-c2cncn2CCO3)CC1]

Prepared from lithium 5,6-dihydrobenzo[f]imidazo[1,5-d][1,4]oxazepine-10-carboxylate 4 and 4-methylsulfonylcyclohexan-1-amine hydrochloride according to General Procedure E to afford the desired product N-(4-(methylsulfonyl)cyclohexyl)-5,6-dihydrobenzo[f]imidazo[1,5-d][1,4]oxazepine-10-carboxamide (124 mg, 0.318 mmol, 38% yield) as a white solid. ¹H NMR (400 MHz, DMSO-D6) δ 8.32-8.17 (m, 2H), 7.77 (d, J=1.1 Hz, 1H), 7.69-7.63 (m, 1H), 7.61-7.56 (m, 1H), 7.04 (d, J=8.5 Hz, 1H), 4.51-4.40 (m, 4H), 4.08-3.68 (m, 1H), 3.19-2.99 (m, 1H), 2.95 (s, 3H), 2.06-1.36 (m, 8H). UPLC-MS (Basic 6 min): rt=1.51 min, m/z (ES+) 390.3 [M+H]⁺.

### N-((1r,4r)-4-((1s,3s)-3-Fluorocyclobutoxy)cyclohexyl)-5,6-dihydrobenzo[f]imidazo[1,5-d][1,4]oxazepine-10-carboxamide 147A

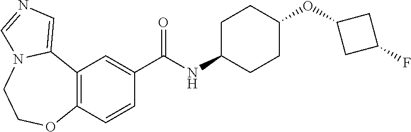 [SMILES: O=C(N[C@H]1CC[C@H](O[C@H]2C[C@@H](F)C2)CC1)c1ccc2c(c1)-c1cncn1CCO2]

Prepared from lithium 5,6-dihydrobenzo[f]imidazo[1,5-d][1,4]oxazepine-10-carboxylate 4 and 4-(3-fluorocyclobutoxy)cyclohexan-1-amine according to General Procedure E to afford the desired product N-((1r,4r)-4-((1s,3s)-3-fluorocyclobutoxy)cyclohexyl)-5,6-dihydrobenzo[f]imidazo[1,5-d][1,4]oxazepine-10-carboxamide (3.0 mg, 0.0074 mmol, 1% yield) as a white solid. ¹H NMR (400 MHz, DMSO-D6) δ 8.22 (d, J=2.2 Hz, 1H), 8.18 (d, J=7.7 Hz, 1H), 7.77 (s, 1H), 7.65 (dd, J=8.6, 2.2 Hz, 1H), 7.60 (s, 1H), 7.03 (d, J=8.6 Hz, 1H), 4.72 (dp, J=56.3, 6.7 Hz, 1H), 4.51-4.39 (m, 4H), 3.80-3.69 (m, 1H), 3.65 (td, J=6.8, 4.0 Hz, 1H), 3.28-3.20 (m, 1H), 2.75 (dtd, J=9.6, 6.5, 3.2 Hz, 2H), 2.10-1.91 (m, 4H), 1.90-1.81 (m, 2H), 1.44-1.31 (m, 2H), 1.25 (q, J=10.6 Hz, 2H). ¹⁹F NMR (376 MHz, DMSO-D6) δ−167.32-−167.66 (m, F). UPLC-MS (Basic 6 min): rt=2.31 min, m/z (ES+) 400.4 [M+H]⁺.

### N-((1r,4r)-4-((1r,3r)-3-Fluorocyclobutoxy)cyclohexyl)-5,6-dihydrobenzo[f]imidazo[1,5-d][1,4]oxazepine-10-carboxamide 148A

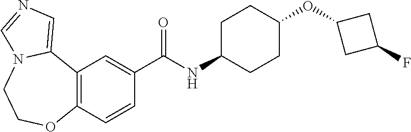 [SMILES: O=C(N[C@H]1CC[C@H](O[C@H]2C[C@H](F)C2)CC1)c1ccc2c(c1)-c1cncn1CCO2]

Also isolated from the same reaction mixture was the other desired product N-((1r,4r)-4-((1r,3r)-3-fluorocyclobutoxy)cyclohexyl)-5,6-dihydrobenzo[f]imidazo[1,5-d][1,4]oxazepine-10-carboxamide (24 mg, 0.0600 mmol, 6% yield) as a white solid. ¹H NMR (400 MHz, DMSO-D6) δ 8.22 (d, J=2.2 Hz, 1H), 8.18 (d, J=7.7 Hz, 1H), 7.77 (d, J=1.1 Hz, 1H), 7.65 (dd, J=8.6, 2.1 Hz, 1H), 7.60 (d, J=1.1 Hz, 1H), 7.03 (d, J=8.6 Hz, 1H), 5.19 (dtt, J=57.0, 6.4, 3.2 Hz, 1H), 4.54-4.41 (m, 4H), 4.41-4.29 (m, 1H), 3.80-3.67 (m, 1H), 3.23 (dq, J=10.2, 5.2 Hz, 1H), 2.40 (dddt, J=21.0, 10.4, 7.0, 3.3 Hz, 2H), 2.25 (dddd, J=22.5, 14.0, 6.3, 5.2 Hz, 2H), 1.99-1.91 (m, 2H), 1.89-1.81 (m, 2H), 1.42-1.34 (m, 2H), 1.32-1.17 (m, 2H). ¹⁹F NMR (376 MHz, DMSO-D6) δ−175.75-−176.28 (m, F). UPLC-MS (Basic 6 min): rt=2.37 min, m/z (ES+) 400.4 [M+H]⁺.

### N-((1s,4s)-4-((1s,3s)-3-Fluorocyclobutoxy)cyclohexyl)-5,6-dihydrobenzo[f]imidazo[1,5-d][1,4]oxazepine-10-carboxamide 149A

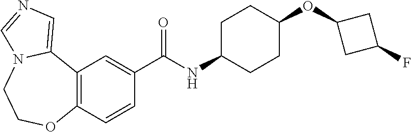 [SMILES: O=C(N[C@H]1CC[C@@H](O[C@H]2C[C@@H](F)C2)CC1)c1ccc2c(c1)-c1cncn1CCO2]

Also isolated from the same reaction mixture was the other desired product N-((1s,4s)-4-((1s,3s)-3-fluorocyclobutoxy)cyclohexyl)-5,6-dihydrobenzo[f]imidazo[1,5-d][1,4]oxazepine-10-carboxamide (9.0 mg, 0.0218 mmol, 2% yield) as a white solid. ¹H NMR (400 MHz, DMSO-D6) δ 8.27 (d, J=2.2 Hz, 1H), 8.23 (d, J=8.0 Hz, 1H), 7.77 (d, J=1.1 Hz, 1H), 7.68 (dd, J=8.6, 2.2 Hz, 1H), 7.63 (d, J=1.1 Hz, 1H), 7.03 (d, J=8.6 Hz, 1H), 4.76 (dp, J=56.4, 6.7 Hz, 1H), 4.51-4.39 (m, 4H), 3.84 (s, 1H), 3.66-3.57 (m, 1H), 3.53 (s, 1H), 2.82-2.70 (m, 2H), 2.14-1.97 (m, 2H), 1.84-1.77 (m, 2H), 1.72-1.65 (m, 2H), 1.62-1.53 (m, 2H), 1.51-1.43 (m, 2H). ¹⁹F NMR (376 MHz, DMSO-D6) δ−166.82-−167.21 (m, F). UPLC-MS (Basic 6 min): rt=2.41 min, m/z (ES+) 400.4 [M+H]⁺.

### N-((1s,4s)-4-((1r,3r)-3-Fluorocyclobutoxy)cyclohexyl)-5,6-dihydrobenzo[f]imidazo[1,5-d][1,4]oxazepine-10-carboxamide 150A

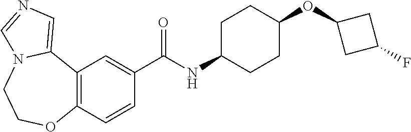 [SMILES: O=C(N[C@H]1CC[C@@H](O[C@H]2C[C@H](F)C2)CC1)c1ccc2c(c1)-c1cncn1CCO2]

Also isolated from the same reaction mixture was the other desired product N-((1s,4s)-4-((1r,3r)-3-fluorocyclobutoxy)cyclohexyl)-5,6-dihydrobenzo[f]imidazo[1,5-d][1,4]oxazepine-10-carboxamide (70 mg, 0.175 mmol, 18% yield) as a white solid. ¹H NMR (400 MHz, DMSO-D6) δ 8.27 (d, J=2.2 Hz, 1H), 8.23 (d, J=7.9 Hz, 1H), 7.77 (d, J=1.1 Hz, 1H), 7.68 (dd, J=8.6, 2.2 Hz, 1H), 7.63 (d, J=1.1 Hz, 1H), 7.03 (d, J=8.6 Hz, 1H), 5.22 (dtt, J=57.0, 6.2, 3.0 Hz, 1H), 4.51-4.39 (m, 4H), 4.34 (ttd, J=7.0, 5.4, 3.2 Hz, 1H), 3.91-3.78 (m, 1H), 3.51 (s, 1H), 2.48-2.36 (m, 2H), 2.36-2.20 (m, 2H), 1.85-1.78 (m, 2H), 1.73-1.54 (m, 4H), 1.51-1.43 (m, 2H). ¹⁹F NMR (376 MHz, DMSO-D6) δ-167.39--176.89 (m, F). UPLC-MS (Basic 6 min): rt=2.47 min, m/z (ES+) 400.4 [M+H]⁺.

### N-(3-(Trifluoromethyl)cyclohexyl)-5,6-dihydrobenzo[f]imidazo[1,5-d][1,4]oxazepine-10-carboxamide 151A

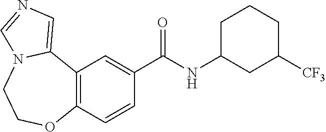 [SMILES: O=C(NC1CCCC(C(F)(F)F)C1)c1ccc2c(c1)-c1cncn1CCO2]

Prepared from lithium 5,6-dihydrobenzo[f]imidazo[1,5-d][1,4]oxazepine-10-carboxylate 4 and 3-(trifluoromethyl)cyclohexanamine according to General Procedure E to afford the desired product N-(3-(trifluoromethyl)cyclohexyl)-5,6-dihydrobenzo[f]imidazo[1,5-d][1,4]oxazepine-10-carboxamide (142 mg, 0.374 mmol, 44% yield) as a white solid. ¹H NMR (400 MHz, DMSO-D6) δ 8.34-8.08 (m, 2H), 7.80-7.75 (m, 1H), 7.71-7.63 (m, 1H), 7.62-7.58 (m, 1H), 7.08-7.01 (m, 1H), 4.51-4.40 (m, 4H), 4.31-3.80 (m, 1H), 2.92-2.51 (m, 1H), 2.11-1.05 (m, 8H). ¹⁹F NMR (376 MHz, DMSO-D6) δ-70.93 (m, CF₃ minor), −70.21 (d, J=8.9 Hz, CF₃ major). UPLC-MS (Basic 6 min): rt=2.66 min, m/z (ES+) 380.4 [M+H]⁺.

### N-((1r,4r)-4-(3,3-Difluorocyclobutoxy)cyclohexyl)-5,6-dihydrobenzo[f]imidazo[1,5-d][1,4]oxazepine-10-carboxamide 152A

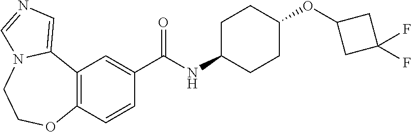 [SMILES: O=C(N[C@H]1CC[C@H](OC2CC(F)(F)C2)CC1)c1ccc2c(c1)-c1cncn1CCO2]

Prepared from lithium 5,6-dihydrobenzo[f]imidazo[1,5-d][1,4]oxazepine-10-carboxylate 4 and 4-(3,3-difluorocyclobutoxy)cyclohexan-1-amine according to General Procedure E to afford the desired product N-((1r,4r)-4-(3,3-difluorocyclobutoxy)cyclohexyl)-5,6-dihydrobenzo[f]imidazo[1,5-d][1,4]oxazepine-10-carboxamide (10 mg, 0.0233 mmol, 2% yield) as a white solid. ¹H NMR (400 MHz, DMSO-D6) δ 8.22 (d, J=2.2 Hz, 1H), 8.19 (d, J=7.7 Hz, 1H), 7.77 (d, J=1.1 Hz, 1H), 7.65 (dd, J=8.6, 2.2 Hz, 1H), 7.60 (d, J=1.1 Hz, 1H), 7.04 (d, J=8.5 Hz, 1H), 4.51-4.39 (m, 4H), 4.16-4.05 (m, 1H), 3.80-3.66 (m, 1H), 3.33-3.24 (m, 1H), 2.97-2.82 (m, 2H), 2.50-2.39 (m, 1H), 2.02-1.93 (m, 2H), 1.90-1.82 (m, 2H), 1.45-1.21 (m, 4H). 1H not observed. ¹⁹F NMR (376 MHz, DMSO-D6) δ-80.75--82.92 (m, F), −94.46--95.96 (m, F). UPLC-MS (Basic 6 min): rt=2.55 min, m/z (ES+) 418.4 [M+H]⁺.

### N-((1s,4s)-4-(3,3-Difluorocyclobutoxy)cyclohexyl)-5,6-dihydrobenzo[f]imidazo[1,5-d][1,4]oxazepine-10-carboxamide 153A

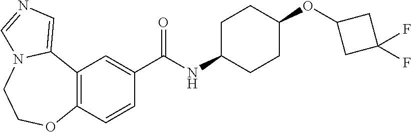 [SMILES: O=C(N[C@H]1CC[C@@H](OC2CC(F)(F)C2)CC1)c1ccc2c(c1)-c1cncn1CCO2]

Also isolated from the same reaction mixture was the other desired product N-((1s,4s)-4-(3,3-difluorocyclobutoxy)cyclohexyl)-5,6-dihydrobenzo[f]imidazo[1,5-d][1,4]oxazepine-10-carboxamide (152 mg, 0.364 mmol, 29% yield) as a white solid. ¹H NMR (400 MHz, DMSO-D6) δ 8.27 (d, J=2.2 Hz, 1H), 8.23 (d, J=7.9 Hz, 1H), 7.77 (d, J=1.1 Hz, 1H), 7.68 (dd, J=8.6, 2.2 Hz, 1H), 7.63 (d, J=1.1 Hz, 1H), 7.03 (d, J=8.6 Hz, 1H), 4.51-4.39 (m, 4H), 4.17-4.03 (m, 1H), 3.91-3.80 (m, 1H), 3.58-3.53 (m, 1H), 2.99-2.84 (m, 2H), 2.57-2.52 (m, 1H), 2.49-2.41 (m, 1H), 1.88-1.80 (m, 2H), 1.73-1.44 (m, 6H). ¹⁹F NMR (376 MHz, DMSO-D6) δ−80.60-−81.78 (m, F), −94.67-−95.81 (m, F). UPLC-MS (Basic 6 min): rt=2.65 min, m/z (ES+) 418.4 [M+H]⁺.

### N-(4,4-Difluorocyclohexyl)-8,9-difluoro-5,6-dihydrobenzo[f]imidazo[1,5-d][1,4]oxazepine-10-carboxamide 154A

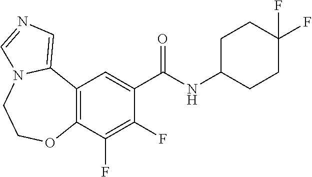 [SMILES: O=C(NC1CCC(F)(F)CC1)c1cc2c(c(F)c1F)OCCn1cncc1-2]

Prepared from lithium 8,9-difluoro-5,6-dihydrobenzo[f]imidazo[1,5-d][1,4]oxazepine-10-carboxylate and 4,4-difluorocyclohexanamine hydrochloride according to General Procedure E to afford the desired product N-(4,4-difluorocyclohexyl)-8,9-difluoro-5,6-dihydrobenzo[f]imidazo[1,5-d][1,4]oxazepine-10-carboxamide (10 mg, 0.0259 mmol, 7% yield) as a white solid. ¹H NMR (400 MHz, DMSO-D6) δ 8.40 (d, J=7.6 Hz, 1H), 7.81 (d, J=1.1 Hz, 1H), 7.74 (dd, J=7.4, 2.3 Hz, 1H), 7.58 (d, J=1.1 Hz, 1H), 4.61-4.50 (m, 4H), 3.96 (d, J=10.4 Hz, 1H), 2.12-1.83 (m, 6H), 1.69-1.55 (m, 2H). ¹⁹F NMR 8-93.39 (d, J=234.8 Hz, F), −97.18 (dt, J=235.4, 28.0 Hz, F), −139.95 (dd, J=21.2, 7.4 Hz, F), −155.46 (dd, J=21.3, 2.3 Hz, F). UPLC-MS (Basic 6 min): rt=2.57 min, m/z (ES+) 384.2 [M−H]⁺.

### 8-Fluoro-N-((1r,4r)-4-(2,2,2-trifluoroethoxy)cyclohexyl)-5,6-dihydrobenzo[f]imidazo[1,5-d][1,4]oxazepine-10-carboxamide 155A

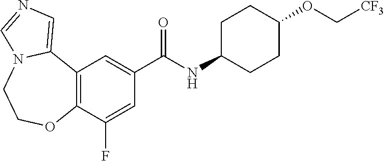 [SMILES: O=C(N[C@H]1CC[C@H](OCC(F)(F)F)CC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 4-(2,2,2-trifluoroethoxy)cyclohexanamine according to General Procedure E to afford the desired product 8-fluoro-N-((1r,4r)-4-(2,2,2-trifluoroethoxy)cyclohexyl)-5,6-dihydrobenzo[f]imidazo[1,5-d][1,4]oxazepine-10-carboxamide (17 mg, 0.0388 mmol, 4% yield) as a white solid. ¹H NMR (400 MHz, DMSO-D6) δ 8.25 (d, J=7.7 Hz, 1H), 8.06 (t, J=1.7 Hz, 1H), 7.81 (d, J=1.1 Hz, 1H), 7.64 (d, J=1.1 Hz, 1H), 7.57 (dd, J=11.4, 2.1 Hz, 1H), 4.53 (s, 4H), 4.08 (q, J=9.4 Hz, 2H), 3.82-3.71 (m, 1H), 3.52-3.39 (m, 1H), 2.10-2.00 (m, 2H), 1.92-1.85 (m, 2H), 1.46-1.21 (m, 4H). ¹⁹F NMR (376 MHz, DMSO-D6) δ-73.08 (t, J=9.5 Hz, CF₃), −130.88 (dd, J=11.7, 1.5 Hz, F). UPLC-MS (Basic 6 min): rt=2.77 min, m/z (ES+) 428.3

### 8-Fluoro-N-((1s,4s)-4-(2,2,2-trifluoroethoxy)cyclohexyl)-5,6-dihydrobenzo[f]imidazo[1,5-d][1,4]oxazepine-10-carboxamide 156A

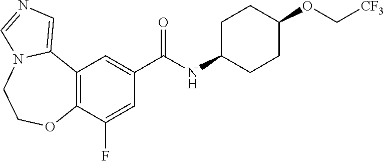 [SMILES: O=C(N[C@H]1CC[C@@H](OCC(F)(F)F)CC1)c1cc(F)c2c(c1)-c1cncn1CCO2]

Also isolated from the same reaction mixture was the other desired product 8-fluoro-N-((1s,4s)-4-(2,2,2-trifluoroethoxy)cyclohexyl)-5,6-dihydrobenzo[f]imidazo[1,5-d][1,4]oxazepine-10-carboxamide (33 mg, 0.0765 mmol, 8% yield) as a white solid. ¹H NMR (400 MHz, DMSO-D6) δ 8.32 (d, J=7.8 Hz, 1H), 8.10 (t, J=1.8 Hz, 1H), 7.81 (d, J=1.1 Hz, 1H), 7.65 (d, J=1.1 Hz, 1H), 7.60 (dd, J=11.5, 2.1 Hz, 1H), 4.53 (s, 4H), 4.05 (q, J=9.5 Hz, 2H), 3.85 (ddq, J=14.5, 10.2, 4.4 Hz, 1H), 3.70 (s, 1H), 1.95-1.87 (m, 2H), 1.73-1.48 (m, 6H). ¹⁹F NMR (376 MHz, DMSO-D6) δ-72.93 (t, J=9.5 Hz, CF₃), −130.95 (dd, J=11.6, 1.5 Hz, F). UPLC-MS (Basic 6 min): rt=2.82 min, m/z (ES+) 428.3 [M+H]⁺.

### 8-Chloro-N-((1r,4r)-4-(2,2,2-trifluoroethoxy)cyclohexyl)-5,6-dihydrobenzo[f]imidazo[1,5-d][1,4]oxazepine-10-carboxamide 157A

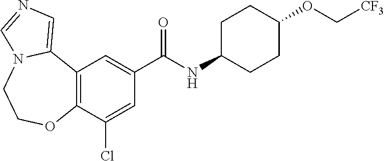 [SMILES: O=C(N[C@H]1CC[C@H](OCC(F)(F)F)CC1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 4-(2,2,2-trifluoroethoxy)cyclohexanamine according to General Procedure E to afford the desired product 8-chloro-N-((1r,4r)-4-(2,2,2-trifluoroethoxy)cyclohexyl)-5,6-dihydrobenzo[f]imidazo[1,5-d][1,4]oxazepine-10-carboxamide (22 mg, 0.0478 mmol, 5% yield) as a white solid. ¹H NMR (400 MHz, DMSO-D6) δ 8.30 (d, J=7.7 Hz, 1H), 8.18 (d, J=2.2 Hz, 1H), 7.83 (d, J=2.3 Hz, 2H), 7.62 (d, J=1.1 Hz, 1H), 4.59-4.48 (m, 4H), 4.07 (q, J=9.4 Hz, 2H), 3.82-3.70 (m, 1H), 3.50-3.40 (m, 1H), 2.09-2.01 (m, 2H), 1.92-1.84 (m, 2H), 1.40-1.28 (m, 4H). ¹⁹F NMR (376 MHz, DMSO-D6) δ-73.08 (t, J=9.4 Hz, CF₃). UPLC-MS (Basic 6 min): rt=2.98 min, m/z (ES+) 444.2, 446.2 [M+H]⁺.

### 8-Chloro-N-((1s,4s)-4-(2,2,2-trifluoroethoxy)cyclohexyl)-5,6-dihydrobenzo[f]imidazo[1,5-d][1,4]oxazepine-10-carboxamide 158A

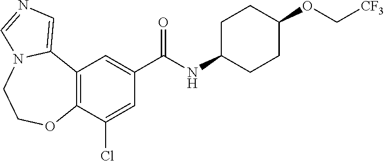 [SMILES: O=C(N[C@H]1CC[C@@H](OCC(F)(F)F)CC1)c1cc(Cl)c2c(c1)-c1cncn1CCO2]

Also isolated from the same reaction mixture was the other desired product 8-chloro-N-((1s,4s)-4-(2,2,2-trifluoroethoxy)cyclohexyl)-5,6-dihydrobenzo[f]imidazo[1,5-d][1,4]oxazepine-10-carboxamide (50 mg, 0.112 mmol, 11% yield) as a white solid. ¹H NMR (400 MHz, DMSO-D6) δ 8.37 (d, J=7.8 Hz, 1H), 8.21 (d, J=2.2 Hz, 1H), 7.86 (d, J=2.1 Hz, 1H), 7.83 (d, J=1.1 Hz, 1H), 7.63 (d, J=1.1 Hz, 1H), 4.59-4.47 (m, 4H), 4.05 (q, J=9.4 Hz, 2H), 3.86 (ddt, J=13.9, 10.0, 5.2 Hz, 1H), 3.70 (s, 1H), 1.95-1.87 (m, 2H), 1.73-1.48 (m, 6H). ¹⁹F NMR (376 MHz, DMSO-D6) δ-72.93 (t, J=9.4 Hz, CF₃). UPLC-MS (Basic 6 min): rt=3.04 min, m/z (ES+) 444.2, 446.2 [M+H]⁺.

### 8-Fluoro-N-(spiro[3.3]heptan-2-yl)-5,6-dihydrobenzo[f]imidazo[1,5-d][1,4]oxazepine-10-carboxamide 159A

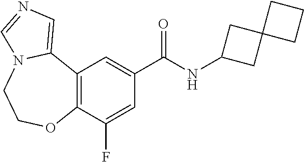 [SMILES: O=C(NC1CC2(CCC2)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and spiro[3.3]heptan-2-amine hydrochloride according to General Procedure E to afford the desired product 8-fluoro-N-(spiro[3.3]heptan-2-yl)-5,6-dihydrobenzo[f]imidazo[1,5-d][1,4]oxazepine-10-carboxamide (49 mg, 0.143 mmol, 36% yield) as a white solid. ¹H NMR (400 MHz, DMSO-D6) δ 8.57 (d, J=7.4 Hz, 1H), 8.07 (t, J=1.8 Hz, 1H), 7.81 (d, J=1.1 Hz, 1H), 7.65 (d, J=1.1 Hz, 1H), 7.56 (dd, J=11.5, 2.1 Hz, 1H), 4.53 (s, 4H), 4.34-4.19 (m, 1H), 2.33 (ddt, J=11.1, 7.5, 2.3 Hz, 2H), 2.10-1.99 (m, 4H), 1.93 (dd, J=8.5, 5.9 Hz, 2H), 1.87-1.74 (m, 2H). ¹⁹F NMR (376 MHz, DMSO-D6) δ-130.81 (dd, J=11.5, 1.5 Hz, F). UPLC-MS (Basic 6 min): rt=2.77 min, m/z (ES+) 342.1 [M+H]⁺.

### 8-Fluoro-N-(2-oxaspiro[3.3]heptan-6-yl)-5,6-dihydrobenzo[f]imidazo[1,5-d][1,4]oxazepine-10-carboxamide 160A

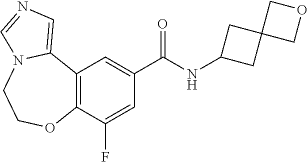 [SMILES: O=C(NC1CC2(COC2)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2-oxaspiro[3.3]heptan-6-amine hydrochloride according to General Procedure E to afford the desired product 8-fluoro-N-(2-oxaspiro[3.3]heptan-6-yl)-5,6-dihydrobenzo[f]imidazo[1,5-d][1,4]oxazepine-10-carboxamide (12 mg, 0.0334 mmol, 9% yield) as a white solid. ¹H NMR (400 MHz, DMSO-D6) δ 8.60 (d, J=7.1 Hz, 1H), 8.06 (t, J=1.8 Hz, 1H), 7.81 (d, J=1.1 Hz, 1H), 7.64 (d, J=1.1 Hz, 1H), 7.55 (dd, J=11.4, 2.1 Hz, 1H), 4.64 (s, 2H), 4.55-4.50 (m, 6H), 4.21 (h, J=8.1 Hz, 1H), 2.63-2.55 (m, 2H), 2.30-2.20 (m, 2H). 19F NMR (376 MHz, DMSO-D6) δ-130.75 (dd, J=11.5, 1.5 Hz, F). UPLC-MS (Basic 6 min): rt=1.68 min, m/z (ES+) 344.1 [M+H]⁺.

### 8-Fluoro-N-(3-(trifluoromethyl)cyclohexyl)-5,6-dihydrobenzo[f]imidazo[1,5-d][1,4]oxazepine-10-carboxamide 161A

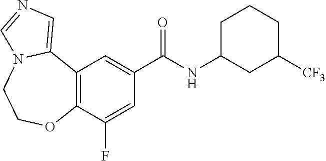 [SMILES: O=C(NC1CCCC(C(F)(F)F)C1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 3-(trifluoromethyl)cyclohexanamine according to General Procedure E to afford the desired product 8-fluoro-N-(3-(trifluoromethyl)cyclohexyl)-5,6-dihydrobenzo[f]imidazo[1,5-d][1,4]oxazepine-10-carboxamide (56 mg, 0.141 mmol, 36% yield) as a white solid. ¹H NMR (400 MHz, DMSO-D6) δ 8.25 (dd, J=90.7, 7.2 Hz, 1H), 8.06 (dt, J=15.7, 1.7 Hz, 1H), 7.82 (dd, J=2.3, 1.1 Hz, 1H), 7.67-7.53 (m, 2H), 4.56-4.51 (m, 4H), 4.32-3.76 (m, 1H), 2.11-1.09 (m, 8H). 1H not observed. ¹⁹F NMR (376 MHz, DMSO-D6) δ-70.94 (d, J=9.5 Hz, CF₃, trans-isomer), −72.22 (d, J=9.0 Hz, CF₃, cis-isomer), −130.81 (dd, J=11.6, 1.6 Hz, F, cis-isomer), —131.09 (dd, J=11.5, 1.5 Hz, F, trans-isomer). UPLC-MS (Basic 6 min): rt=2.82 min, m/z (ES+) 398.2 [M+H]⁺.

### 8-Fluoro-N-(1-methylcyclohexyl)-5,6-dihydrobenzo[f]imidazo[1,5-d][1,4]oxazepine-10-carboxamide 162A

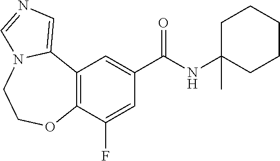 [SMILES: CC1(NC(=O)c2cc(F)c3c(c2)-c2cncn2CCO3)CCCCC1]

Prepared from lithium 8-fluoro-5,6-dihydrobenzo[f]imidazo[1,5-d][1,4]oxazepine-10-carboxylate and 1-amino-1-methylcyclohexane hydrochloride to General Procedure E to afford the desired product 8-fluoro-N-(1-methylcyclohexyl)-5,6-dihydrobenzo[f]imidazo[1,5-d][1,4]oxazepine-10-carboxamide (8.0 mg, 0.0227 mmol, 6% yield) as a white solid. ¹H NMR (400 MHz, DMSO-D6) δ 8.00 (t, J=1.8 Hz, 1H), 7.81 (d, J=1.1 Hz, 1H), 7.63 (d, J=1.1 Hz, 1H), 7.58 (dd, J=11.4, 2.0 Hz, 1H), 7.55 (s, 1H), 4.53 (s, 4H), 2.28-2.18 (m, 2H), 1.52-1.43 (m, 5H), 1.38 (dd, J=13.3, 4.3 Hz, 2H), 1.34 (s, 3H), 1.33-1.20 (m, 1H). ¹⁹F NMR (376 MHz, DMSO-D6) δ-131.14 (dd, J=11.4, 1.5 Hz, F). UPLC-MS (Basic 6 min): rt=2.85 min, m/z (ES+) 344.2 [M+H]⁺.

### 8-Chloro-N-(1-methylcyclohexyl)-5,6-dihydrobenzo[f]imidazo[1,5-d][1,4]oxazepine-10-carboxamide 163A

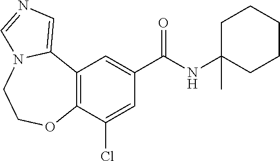 [SMILES: CC1(NC(=O)c2cc(Cl)c3c(c2)-c2cncn2CCO3)CCCCC1]

Prepared from lithium 8-chloro-5,6-dihydrobenzo[f]imidazo[1,5-d][1,4]oxazepine-10-carboxylate and 1-amino-1-methylcyclohexane hydrochloride according to General Procedure E to afford the desired product 8-chloro-N-(1-methylcyclohexyl)-5,6-dihydrobenzo[f]imidazo[1,5-d][1,4]oxazepine-10-carboxamide (29 mg, 0.0798 mmol, 22% yield) as a white solid. ¹H NMR (400 MHz, DMSO-D6) δ 8.12 (d, J=2.2 Hz, 1H), 7.83 (dd, J=3.3, 1.6 Hz, 2H), 7.64-7.58 (m, 2H), 4.58-4.47 (m, 4H), 2.28-2.18 (m, 2H), 1.50-1.40 (m, 5H), 1.42-1.34 (m, 2H), 1.34 (s, 3H), 1.32-1.21 (m, 1H). UPLC-MS (Basic 6 min): rt=3.10 min, m/z (ES+) 360.1, 362.1 [M+H]⁺.

### 8-Fluoro-N-(4-(2,2,2-trifluoroethyl)cyclohexyl)-5,6-dihydrobenzo[f]imidazo[1,5-d][1,4]oxazepine-10-carboxamide 164A

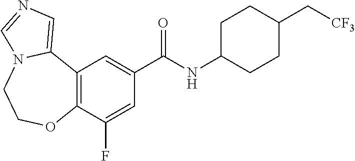 [SMILES: O=C(NC1CCC(CC(F)(F)F)CC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 4-(2,2,2-trifluoroethyl)cyclohexanamine according to General Procedure E to afford the desired product 8-fluoro-N-(4-(2,2,2-trifluoroethyl)cyclohexyl)-5,6-dihydrobenzo[f]imidazo[1,5-d][1,4]oxazepine-10-carboxamide (44 mg, 0.107 mmol, 18% yield) as a white solid. ¹H NMR (400 MHz, DMSO-D6) δ 8.20 (dd, J=69.2, 7.5 Hz, 1H), 8.06 (dt, J=11.9, 1.8 Hz, 1H), 7.82 (dd, J=2.5, 1.1 Hz, 1H), 7.64 (dd, J=3.9, 1.1 Hz, 1H), 7.58 (ddd, J=11.5, 6.7, 2.1 Hz, 1H), 4.56-4.51 (m, 4H), 3.96-3.67 (m, 1H), 2.31-2.19 (m, 2H), 1.97-1.77 (m, 2H), 1.73-1.09 (m, 7H). ¹⁹F NMR (376 MHz, DMSO-D6) δ-61.54 (t, J=12.3 Hz, CF₃ trans-isomer), −61.99 (t, J=12.1 Hz, CF₃ cis-isomer), −130.89 (d, J=11.3 Hz, F trans-isomer), −130.93-−131.02 (m, F, cis-isomer). UPLC-MS (Basic 6 min): rt=2.99 min, m/z (ES+) 412.2 [M+H]⁺.

### 8-Chloro-N-(4-(2,2,2-trifluoroethyl)cyclohexyl)-5,6-dihydrobenzo[f]imidazo[1,5-d][1,4]oxazepine-10-carboxamide 165A

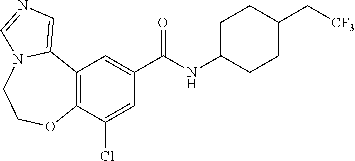 [SMILES: O=C(NC1CCC(CC(F)(F)F)CC1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 4-(2,2,2-trifluoroethyl)cyclohexanamine according to General Procedure E to afford the desired product 8-chloro-N-(4-(2,2,2-trifluoroethyl)cyclohexyl)-5,6-dihydrobenzo[f]imidazo[1,5-d][1,4]oxazepine-10-carboxamide (44 mg, 0.107 mmol, 18% yield) as a white solid. ¹H NMR (400 MHz, DMSO-D6) δ 8.36-8.13 (m, 2H), 7.83 (q, J=2.1 Hz, 2H), 7.62 (dd, J=3.5, 1.1 Hz, 1H), 4.59-4.47 (m, 4H), 3.96-3.65 (m, 1H), 2.37-2.13 (m, 2H), 1.94-1.80 (m, 2H), 1.72-1.12 (m, 7H). ¹⁹F NMR (376 MHz, DMSO-D6) δ-61.54 (t, J=12.2 Hz, CF₃ trans-isomer), −61.99 (t, J=12.1 Hz, CF₃ cis-isomer). UPLC-MS (Basic 6 min): rt=3.18 min, m/z (ES+) 428.2, 430.2 [M+H]⁺.

### N-((1r,4r)-4-(3,3-Difluorocyclobutoxy)cyclohexyl)-8-fluoro-5,6-dihydrobenzo[f]imidazo[1,5-d][1,4]oxazepine-10-carboxamide 166A

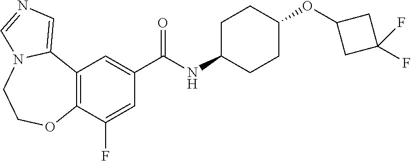 [SMILES: O=C(N[C@H]1CC[C@H](OC2CC(F)(F)C2)CC1)c1cc(F)c2c(c1)-c1cncn1CCO2]

Prepared from lithium 8-fluoro-5,6-dihydrobenzo[f]imidazo[1,5-d][1,4]oxazepine-10-carboxylate and 4-(3,3-difluorocyclobutoxy)cyclohexan-1-amine according to General Procedure E to afford the desired product N-((1r,4r)-4-(3,3-difluorocyclobutoxy)cyclohexyl)-8-fluoro-5,6-dihydrobenzo[f]imidazo[1,5-d][1,4]oxazepine-10-carboxamide (13 mg, 0.0285 mmol, 5% yield) as a white solid. ¹H NMR (400 MHz, DMSO-D6) δ 8.25 (d, J=7.7 Hz, 1H), 8.06 (t, J=1.8 Hz, 1H), 7.81 (d, J=1.1 Hz, 1H), 7.64 (d, J=1.1 Hz, 1H), 7.57 (dd, J=11.4, 2.1 Hz, 1H), 4.53 (s, 4H), 4.11 (q, J=6.1 Hz, 1H), 3.74 (dt, J=7.6, 3.9 Hz, 1H), 2.97-2.82 (m, 2H), 2.04-1.92 (m, 2H), 1.90-1.81 (m, 2H), 1.44-1.21 (m, 4H). 3H not observed. ¹⁹F NMR (376 MHz, DMSO-D6) δ−81.14-−81.86 (m, F), −94.76-−95.50 (m, F), −130.86--130.93 (m, F). UPLC-MS (Basic 6 min): rt=2.71 min, m/z (ES+) 436.3 [M+H]⁺.

### N-((1s,4s)-4-(3,3-Difluorocyclobutoxy)cyclohexyl)-8-fluoro-5,6-dihydrobenzo[f]imidazo[1,5-d][1,4]oxazepine-10-carboxamide 167A

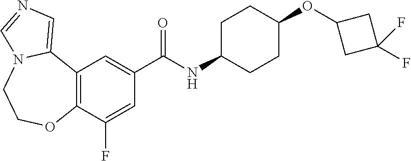 [SMILES: O=C(N[C@H]1CC[C@@H](OC2CC(F)(F)C2)CC1)c1cc(F)c2c(c1)-c1cncn1CCO2]

Also isolated from the same reaction mixture was the other desired product N-((1s,4s)-4-(3,3-difluorocyclobutoxy)cyclohexyl)-8-fluoro-5,6-dihydrobenzo[f]imidazo[1,5-d][1,4]oxazepine-10-carboxamide (38 mg, 0.0872 mmol, 15% yield) as a white solid. ¹H NMR (400 MHz, DMSO-D6) δ 8.29 (d, J=7.9 Hz, 1H), 8.10 (t, J=1.8 Hz, 1H), 7.81 (d, J=1.1 Hz, 1H), 7.66 (d, J=1.1 Hz, 1H), 7.59 (dd, J=11.5, 2.1 Hz, 1H), 4.53 (s, 4H), 4.15-4.01 (m, 1H), 3.92-3.77 (m, 1H), 3.56 (s, 1H), 2.99-2.85 (m, 2H), 1.92-1.75 (m, 2H), 1.75-1.40 (m, 6H). 2H not observed. ¹⁹F NMR (376 MHz, DMSO-D6) δ−80.68-−81.55 (m, F), −94.80-−95.66 (m, F), −130.92 (dd, J=11.6, 1.5 Hz, F). UPLC-MS (Basic 6 min): rt=2.81 min, m/z (ES+) 436.2 [M+H]⁺.

### 8-Fluoro-N-((1s,4s)-4-(3,3,3-trifluoro-2,2-dimethylpropoxy)cyclohexyl)-5,6-dihydrobenzo[f]imidazo[1,5-d][1,4]oxazepine-10-carboxamide 168A

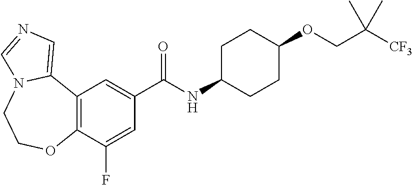 [SMILES: CC(C)(CO[C@H]1CC[C@@H](NC(=O)c2cc(F)c3c(c2)-c2cncn2CCO3)CC1)C(F)(F)F]

Prepared from lithium 8-fluoro-5,6-dihydrobenzo[f]imidazo[1,5-d][1,4]oxazepine-10-carboxylate and 4-(3,3,3-trifluoro-2,2-dimethylpropoxy)cyclohexan-1-amine according to General Procedure E to afford the desired product 8-fluoro-N-((1s,4s)-4-(3,3,3-trifluoro-2,2-dimethylpropoxy)cyclohexyl)-5,6-dihydrobenzo[f]imidazo[1,5-d][1,4]oxazepine-10-carboxamide (23 mg, 0.0489 mmol, 11% yield) as a white solid. ¹H NMR (400 MHz, DMSO-D6) δ 8.30 (d, J=7.7 Hz, 1H), 8.08 (t, J=1.8 Hz, 1H), 7.81 (d, J=1.1 Hz, 1H), 7.64 (d, J=1.1 Hz, 1H), 7.61 (dd, J=11.5, 2.1 Hz, 1H), 4.53 (s, 4H), 3.86-3.75 (m, 1H), 3.48 (s, 1H), 1.92-1.84 (m, 2H), 1.72-1.56 (m, 4H), 1.53-1.43 (m, 2H), 1.14 (s, 6H). 2H not observed. ¹⁹F NMR (376 MHz, DMSO-D6) δ-74.98 (s, CF₃), −131.02 (dd, J=11.6, 1.5 Hz, F). UPLC-MS (Basic 6 min): rt=3.49 min, m/z (ES+) 470.3 [M+H]⁺.

### 8-Fluoro-N-(3-methyl-1,1-dioxidotetrahydrothiophen-3-yl)-5,6-dihydrobenzo[f]imidazo[1,5-d][1,4]oxazepine-10-carboxamide 169A

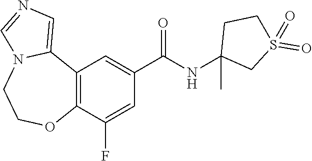 [SMILES: CC1(NC(=O)c2cc(F)c3c(c2)-c2cncn2CCO3)CCS(=O)(=O)C1]

Prepared from lithium 8-fluoro-5,6-dihydrobenzo[f]imidazo[1,5-d][1,4]oxazepine-10-carboxylate and 3-methyl-1,1-dioxothiolan-3-amine hydrochloride according to General Procedure E to afford the desired product 8-fluoro-N-(3-methyl-1,1-dioxidotetrahydrothiophen-3-yl)-5,6-dihydrobenzo[f]imidazo[1,5-d][1,4]oxazepine-10-carboxamide (35 mg, 0.0922 mmol, 23% yield) as a white solid. ¹H NMR (400 MHz, DMSO-D6) δ 8.36 (s, 1H), 8.03 (t, J=1.8 Hz, 1H), 7.82 (d, J=1.0 Hz, 1H), 7.62 (d, J=1.1 Hz, 1H), 7.57 (dd, J=11.3, 2.1 Hz, 1H), 4.54 (s, 4H), 3.93 (dd, J=13.7, 1.7 Hz, 1H), 3.21 (d, J=13.6 Hz, 1H), 2.80-2.69 (m, 1H), 2.28-2.15 (m, 1H), 1.57 (s, 3H). 2H not observed. ¹⁹F NMR (376 MHz, DMSO-D6) δ-130.82 (dd, J=11.6, 1.6 Hz, F). UPLC-MS (Basic 4 min): rt=1.66 min, m/z (ES+) 380.1 [M+H]⁺.

### 8-Chloro-N-((1r,4r)-4-(3,3,3-trifluoro-2,2-dimethylpropoxy)cyclohexyl)-5,6-dihydrobenzo[f]imidazo[1,5-d][1,4]oxazepine-10-carboxamide 170A

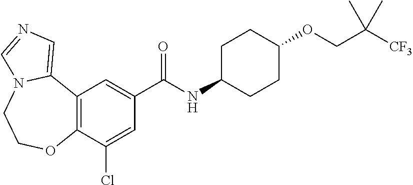 [SMILES: CC(C)(CO[C@H]1CC[C@H](NC(=O)c2cc(Cl)c3c(c2)-c2cncn2CCO3)CC1)C(F)(F)F]

Prepared from lithium 8-chloro-5,6-dihydrobenzo[f]imidazo[1,5-d][1,4]oxazepine-10-carboxylate and 4-(3,3,3-trifluoro-2,2-dimethylpropoxy)cyclohexan-1-amine according to General Procedure E to afford the desired product 8-chloro-N-((1r,4r)-4-(3,3,3-trifluoro-2,2-dimethylpropoxy)cyclohexyl)-5,6-dihydrobenzo[f]imidazo[1,5-d][1,4]oxazepine-10-carboxamide (8.0 mg, 0.0162 mmol, 4% yield) as a white solid. ¹H NMR (400 MHz, DMSO-D6) δ 8.30 (d, J=7.7 Hz, 1H), 8.18 (d, J=2.1 Hz, 1H), 7.85-7.80 (m, 2H), 7.62 (d, J=1.1 Hz, 1H), 4.58-4.48 (m, 4H), 3.83-3.68 (m, 1H), 3.42 (s, 2H), 3.28-3.19 (m, 1H), 2.06-1.98 (m, 2H), 1.91-1.83 (m, 2H), 1.42-1.34 (m, 2H), 1.28-1.22 (m, 2H), 1.09 (s, 6H). ¹⁹F NMR (376 MHz, DMSO-D6) δ-75.04 (s, CF₃). UPLC-MS (Basic 6 min): rt=3.59 min, m/z (ES+) 486.2, 488.2 [M+H]⁺.

### 8-Chloro-N-((1s,4s)-4-(3,3,3-trifluoro-2,2-dimethylpropoxy)cyclohexyl)-5,6-dihydrobenzo[f]imidazo[1,5-d][1,4]oxazepine-10-carboxamide 171A

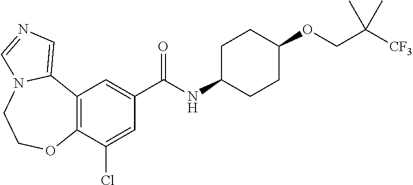 [SMILES: CC(C)(CO[C@H]1CC[C@@H](NC(=O)c2cc(Cl)c3c(c2)-c2cncn2CCO3)CC1)C(F)(F)F]

Also isolated from the same reaction mixture was the other desired product 8-chloro-N-((1s,4s)-4-(3,3,3-trifluoro-2,2-dimethylpropoxy)cyclohexyl)-5,6-dihydrobenzo[f]imidazo[1,5-d][1,4]oxazepine-10-carboxamide (67 mg, 0.148 mmol, 18% yield) as a white solid. ¹H NMR (400 MHz, DMSO-D6) δ 8.34 (d, J=7.7 Hz, 1H), 8.19 (d, J=2.1 Hz, 1H), 7.86 (dd, J=2.2, 0.7 Hz, 1H), 7.83 (s, 1H), 7.62 (d, J=1.0 Hz, 1H), 4.59-4.47 (m, 4H), 3.82 (dd, J=12.5, 6.8 Hz, 1H), 3.48 (s, 1H), 3.36 (s, 2H), 1.92-1.83 (m, 2H), 1.74-1.56 (m, 4H), 1.53-1.43 (m, 2H), 1.14 (s, 6H). ¹⁹F NMR (376 MHz, DMSO-D6) δ-74.98 (s, CF₃). UPLC-MS (Basic 6 min): rt=3.68 min, m/z (ES+) 486.2, 488.2 [M+H]⁺.

### N-((1r,4r)-4-(3,3-difluorocyclobutoxy)cyclohexyl)-8-chloro-5,6-dihydrobenzo[f]imidazo[1,5-d][1,4]oxazepine-10-carboxamide 172A

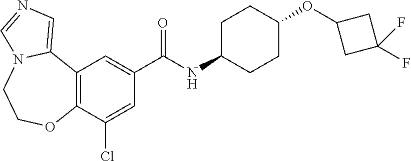 [SMILES: O=C(N[C@H]1CC[C@H](OC2CC(F)(F)C2)CC1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 4-(3,3-difluorocyclobutoxy)cyclohexan-1-amine according to General Procedure E to afford the desired product N-((1r,4r)-4-(3,3-difluorocyclobutoxy)cyclohexyl)-8-chloro-5,6-dihydrobenzo[f]imidazo[1,5-d][1,4]oxazepine-10-carboxamide (11 mg, 0.0243 mmol, 4% yield) as a white solid. ¹H NMR (400 MHz, DMSO-D6) δ 8.30 (d, J=7.7 Hz, 1H), 8.17 (d, J=2.2 Hz, 1H), 7.82 (d, J=2.2 Hz, 2H), 7.62 (d, J=1.1 Hz, 1H), 4.58-4.47 (m, 4H), 4.16-4.03 (m, 1H), 3.80-3.66 (m, 1H), 2.97-2.82 (m, 2H), 2.02-1.96 (m, 2H), 1.91-1.81 (m, 2H), 1.44-1.20 (m, 4H). 3H not observed. ¹⁹F NMR (376 MHz, DMSO-D6) δ−80.91-−82.05 (m, F), −94.65-−95.61 (m, F). UPLC-MS (Basic 6 min): rt=2.92 min, m/z (ES+) 452.2, 454.2 [M+H]⁺.

### N-((1s,4s)-4-(3,3-difluorocyclobutoxy)cyclohexyl)-8-chloro-5,6-dihydrobenzo[f]imidazo[1,5-d][1,4]269xazepane-10-carboxamide 173A

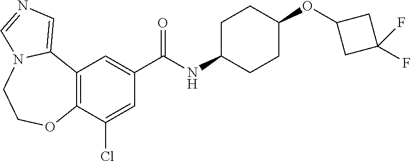 [SMILES: O=C(N[C@H]1CC[C@@H](OC2CC(F)(F)C2)CC1)c1cc(Cl)c2c(c1)-c1cncn1CCO2]

Also isolated from the same reaction mixture was the other desired product N-((1s,4s)-4-(3,3-difluorocyclobutoxy)cyclohexyl)-8-chloro-5,6-dihydrobenzo[f]imidazo[1,5-d][1,4]269xazepane-10-carboxamide (37 mg, 0.0818 mmol, 14% yield) as a white solid. ¹H NMR (400 MHz, DMSO-D6) δ 8.33 (d, J=7.9 Hz, 1H), 8.22 (d, J=2.2 Hz, 1H), 7.86 (d, J=2.1 Hz, 1H), 7.83 (d, J=1.1 Hz, 1H), 7.64 (d, J=1.1 Hz, 1H), 4.59-4.47 (m, 4H), 4.15-4.01 (m, 1H), 3.91-3.79 (m, 1H), 3.59-3.52 (m, 1H), 3.00-2.83 (m, 2H), 1.88-1.79 (m, 2H), 1.73-1.44 (m, 6H). 2H not observed. ¹⁹F NMR (376 MHz, DMSO-D6) δ−80.34--81.93 (m, F), −94.41--96.20 (m, F). UPLC-MS (Basic 6 min): rt=3.01 min, m/z (ES+) 452.2, 454.2 [M+H]⁺.

### 8-Fluoro-N-(trans-2-methylcyclohexyl)-5,6-dihydrobenzo[f]imidazo[1,5-d][1,4]oxazepine-10-carboxamide 174A

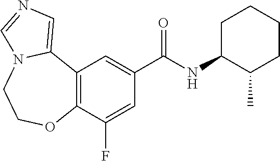 [SMILES: C[C@H]1CCCC[C@@H]1NC(=O)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2-methylcyclohexylamine according to General Procedure E to afford the desired product 8-fluoro-N-(trans-2-methylcyclohexyl)-5,6-dihydrobenzo[f]imidazo[1,5-d][1,4]oxazepine-10-carboxamide (30 mg, 0.0873 mmol, 22% yield) as a white solid. ¹H NMR (400 MHz, DMSO-D6) δ 8.21 (d, J=8.7 Hz, 1H), 8.11-8.05 (m, 1H), 7.82 (d, J=1.1 Hz, 1H), 7.65 (d, J=1.1 Hz, 1H), 7.59 (dd, J=11.5, 2.1 Hz, 1H), 4.53 (s, 4H), 3.55-3.43 (m, 1H), 1.76 (d, J=16.4 Hz, 3H), 1.65 (d, J=11.8 Hz, 1H), 1.59-1.43 (m, 1H), 1.38-1.13 (m, 3H), 1.13-0.97 (m, 1H), 0.86 (d, J=6.4 Hz, 3H). ¹⁹F NMR (376 MHz, DMSO-D6) δ-130.89 (dd, J=11.6, 1.6 Hz, F). UPLC-MS (Basic 6 min): rt=2.70 min, m/z (ES+) 344.2 [M+H]⁺. The absolute stereochemistry of 174A is drawn arbitrarily in the depiction above and is intended to reflect the relative stereochemistry of the compound.

### 8-Fluoro-N-(cis-2-methylcyclohexyl)-5,6-dihydrobenzo[f]imidazo[1,5-d][1,4]oxazepine-10-carboxamide 175A

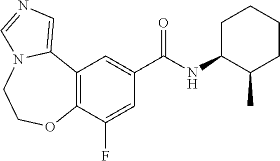 [SMILES: C[C@@H]1CCCC[C@@H]1NC(=O)c1cc(F)c2c(c1)-c1cncn1CCO2]

Also isolated from the same reaction mixture was the other desired product 8-fluoro-N-(cis-2-methylcyclohexyl)-5,6-dihydrobenzo[f]imidazo[1,5-d][1,4]oxazepine-10-carboxamide (8.0 mg, 0.0231 mmol, 6% yield) as a white solid. ¹H NMR (400 MHz, DMSO-D6) δ 8.04 (t, J=1.8 Hz, 1H), 8.01 (d, J=8.2 Hz, 1H), 7.82 (d, J=1.2 Hz, 1H), 7.67-7.58 (m, 2H), 4.53 (s, 4H), 4.05 (dt, J=8.4, 4.3 Hz, 1H), 1.97 (s, 1H), 1.67 (t, J=9.3 Hz, 2H), 1.61-1.41 (m, 4H), 1.34 (d, J=9.1 Hz, 2H), 0.87 (d, J=7.0 Hz, 3H). ¹⁹F NMR (376 MHz, DMSO-D6) δ−131.06-−131.16 (m, F). UPLC-MS (Basic 6 min): rt=2.77 min, m/z (ES+) 344.2 [M+H]⁺. The absolute stereochemistry of 175A is drawn arbitrarily in the depiction above and is intended to reflect the relative stereochemistry of the compound.

### N-(trans-2-trans-6-Dimethylcyclohexyl)-8-fluoro-5,6-dihydrobenzo[f]imidazo[1,5-d][1,4]oxazepine-10-carboxamide 176A

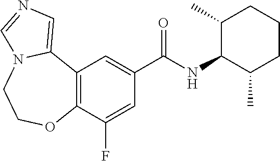 [SMILES: C[C@@H]1CCC[C@H](C)[C@H]1NC(=O)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2,6-dimethylcyclohexan-1-amine hydrochloride according to General Procedure E to afford the desired product N-(trans-2-trans-6-dimethylcyclohexyl)-8-fluoro-5,6-dihydrobenzo[f]imidazo[1,5-d][1,4]oxazepine-10-carboxamide (5.0 mg, 0.0140 mmol, 4% yield) as a white solid. ¹H NMR (400 MHz, DMSO-D6) δ 8.17-8.00 (m, 2H), 7.82 (d, J=1.0 Hz, 1H), 7.66 (d, J=1.1 Hz, 1H), 7.61 (dd, J=11.5, 2.1 Hz, 1H), 4.53 (s, 4H), 1.79-1.71 (m, 2H), 1.64 (d, J=13.0 Hz, 1H), 1.50 (s, 2H), 1.34-1.22 (m, 1H), 1.08 (p, J=13.7 Hz, 2H), 0.82 (d, J=6.5 Hz, 6H). ¹H not observed. ¹⁹F NMR (376 MHz, DMSO-D6) δ−130.82-−130.89 (m, F). UPLC-MS (Basic 6 min): rt=2.88 min, m/z (ES+) 358.2 [M+H]⁺.

### N-(trans-2-cis-6-Dimethylcyclohexyl)-8-fluoro-5,6-dihydrobenzo[f]imidazo[1,5-d][1,4]oxazepine-10-carboxamide 177A

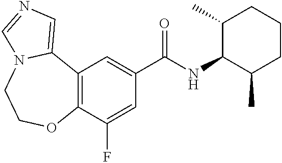

Also isolated from the same reaction mixture was the other desired product N-(trans-2-cis-6-dimethylcyclohexyl)-8-fluoro-5,6-dihydrobenzo[f]imidazo[1,5-d][1,4]oxazepine-10-carboxamide (11 mg, 0.0305 mmol, 8% yield) as a white solid. ¹H NMR (400 MHz, DMSO-D6) δ 8.18 (d, J=8.3 Hz, 1H), 8.11-8.05 (m, 1H), 7.82 (d, J=1.1 Hz, 1H), 7.64 (d, J=1.1 Hz, 1H), 7.61 (dd, J=11.5, 2.1 Hz, 1H), 4.53 (s, 4H), 3.63 (ddd, J=10.7, 8.3, 4.5 Hz, 1H), 2.14-2.02 (m, 1H), 1.86-1.72 (m, 2H), 1.60-1.33 (m, 4H), 1.10-0.97 (m, 1H), 0.91 (d, J=7.2 Hz, 3H), 0.87 (d, J=6.3 Hz, 3H). ¹⁹F NMR (376 MHz, DMSO-D6) δ-130.99 (dd, J=11.5, 1.5 Hz, F). UPLC-MS (Basic 6 min): rt=2.92 min, m/z (ES+) 358.2 [M+H]⁺. The absolute stereochemistry of 177A is drawn arbitrarily in the depiction above and is intended to reflect the relative stereochemistry of the compound.

### N-(cis-2-cis-6-Dimethylcyclohexyl)-8-fluoro-5,6-dihydrobenzo[f]imidazo[1,5-d][1,4]oxazepine-10-carboxamide 178A

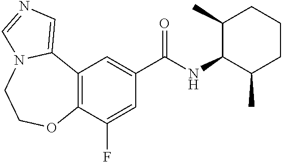 [SMILES: C[C@@H]1CCC[C@H](C)[C@@H]1NC(=O)c1cc(F)c2c(c1)-c1cncn1CCO2]

Also isolated from the same reaction mixture was the other desired product N-(cis-2-cis-6-dimethylcyclohexyl)-8-fluoro-5,6-dihydrobenzo[f]imidazo[1,5-d][1,4]oxazepine-10-carboxamide (5.0 mg, 0.0138 mmol, 4% yield) as a white solid. ¹H NMR (400 MHz, DMSO-D6) δ 8.03-7.97 (m, 1H), 7.82 (d, J=1.1 Hz, 1H), 7.66 (dd, J=11.4, 2.1 Hz, 1H), 7.59 (d, J=1.1 Hz, 1H), 7.45 (d, J=10.2 Hz, 1H), 4.54 (s, 4H), 4.29-4.21 (m, 1H), 1.74-1.60 (m, 3H), 1.49-1.21 (m, 5H), 0.79 (d, J=6.7 Hz, 6H). ¹⁹F NMR (376 MHz, DMSO-D6) δ-131.22 (dd, J=11.4, 1.6 Hz, F). UPLC-MS (Basic 6 min): rt=2.99 min, m/z (ES+) 358.2 [M+H]⁺.

### N-((1r,4r)-4-(2-Methoxyethoxy)cyclohexyl)-8,9-dimethyl-5,6-dihydrobenzo[f]imidazo[1,5-d][1,4]oxazepine-10-carboxamide 179A

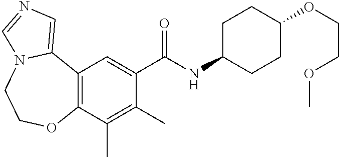 [SMILES: COCCO[C@H]1CC[C@H](NC(=O)c2cc3c(c(C)c2C)OCCn2cncc2-3)CC1]

Prepared from lithium 8,9-dimethyl-5,6-dihydrobenzo[f]imidazo[1,5-d][1,4]oxazepine-10-carboxylate and trans-4-(2-methoxyethoxy)cyclohexanamine according to General Procedure E to afford the desired product N-((1r,4r)-4-(2-methoxyethoxy)cyclohexyl)-8,9-dimethyl-5,6-dihydrobenzo[f]imidazo[1,5-d][1,4]oxazepine-10-carboxamide (38 mg, 0.0901 mmol, 32% yield) as a white solid. ¹H NMR (400 MHz, DMSO-D6) δ 8.11 (d, J=7.9 Hz, 1H), 7.75 (d, J=1.1 Hz, 1H), 7.45-7.36 (m, 2H), 4.45-4.35 (m, 4H), 3.75-3.63 (m, 1H), 3.55-3.48 (m, 2H), 3.45-3.37 (m, 2H), 3.27-3.16 (m, 4H), 2.19 (s, 3H), 2.16 (s, 3H), 2.03-1.95 (m, 2H), 1.93-1.85 (m, 2H), 1.37-1.12 (m, 4H). UPLC-MS (Basic 6 min): rt=2.26 min, m/z (ES+) 414.3 [M−H]⁺.

### N-(4,4-Difluorocyclohexyl)-8,9-dimethyl-5,6-dihydrobenzo[f]imidazo[1,5-d][1,4]oxazepine-10-carboxamide 180A

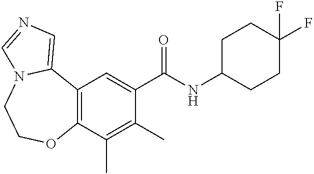 [SMILES: Cc1c(C(=O)NC2CCC(F)(F)CC2)cc2c(c1C)OCCn1cncc1-2]

Prepared from lithium 8,9-dimethyl-5,6-dihydrobenzo[f]imidazo[1,5-d][1,4]oxazepine-10-carboxylate and 4,4-difluorocyclohexanamine hydrochloride according to General Procedure E to afford the desired product N-(4,4-difluorocyclohexyl)-8,9-dimethyl-5,6-dihydrobenzo[f]imidazo[1,5-d][1,4]oxazepine-10-carboxamide (35 mg, 0.0931 mmol, 33% yield) as a white solid. ¹H NMR (400 MHz, DMSO-D6) δ 8.24 (d, J=7.7 Hz, 1H), 7.76 (s, 1H), 7.45 (s, 1H), 7.41 (d, J=1.1 Hz, 1H), 4.45-4.36 (m, 4H), 3.94 (d, J=10.1 Hz, 1H), 2.20 (s, 3H), 2.17 (s, 3H), 2.08-2.01 (m, 3H), 1.93-1.86 (m, 3H), 1.66-1.52 (m, 2H). ¹⁹F NMR (376 MHz, DMSO-D6) 8-93.27 (d, J=234.4 Hz, F), −96.53--97.69 (m, F). UPLC-MS (Basic 6 min): rt=2.51 min, m/z (ES+) 376.2 [M−H]⁺.

### 8-Chloro-N-(3-(trifluoromethyl)cyclohexyl)-5,6-dihydrobenzo[f]imidazo[1,5-d][1,4]oxazepine-10-carboxamide 181A

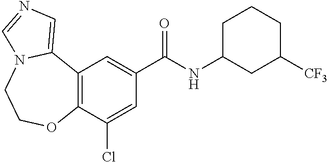 [SMILES: O=C(NC1CCCC(C(F)(F)F)C1)c1cc(Cl)c2c(c1)-c1cncn1CCO2]

Prepared from lithium 8-chloro-5,6-dihydrobenzo[f]imidazo[1,5-d][1,4]oxazepine-10-carboxylate and 3-(trifluoromethyl)cyclohexanamine according to General Procedure E to afford the desired product 8-chloro-N-(3-(trifluoromethyl)cyclohexyl)-5,6-dihydrobenzo[f]imidazo[1,5-d][1,4]oxazepine-10-carboxamide (40 mg, 0.0966 mmol, 26% yield) as a white solid. ¹H NMR (400 MHz, DMSO-D6) δ 8.43-8.14 (m, 2H), 7.90-7.81 (m, 2H), 7.62 (dd, J=6.7, 1.1 Hz, 1H), 4.59-4.48 (m, 4H), 4.28-3.83 (m, 1H), 2.06-1.11 (m, 8H). 1H not observed. ¹⁹F NMR (376 MHz, DMSO-D6) δ-70.96 (d, J=9.5 Hz, CF₃ cis-isomer), −72.22 (d, J=8.9 Hz, CF₃ trans-isomer). UPLC-MS (Basic 6 min): rt=3.03 min, m/z (ES+) 414.2, 416.1 [M+H]⁺.

### 8-Chloro-N-(2-methyl-4-(2,2,2-trifluoroethyl)phenyl)-5,6-dihydrobenzo[f]imidazo[1,5-d][1,4]oxazepine-10-carboxamide 182A

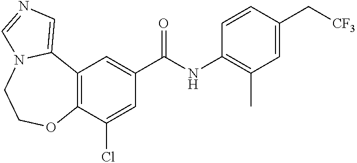 [SMILES: Cc1cc(CC(F)(F)F)ccc1NC(=O)c1cc(Cl)c2c(c1)-c1cncn1CCO2]

Prepared from lithium 8-chloro-5,6-dihydrobenzo[f]imidazo[1,5-d][1,4]oxazepine-10-carboxylate and an inseparable mixture of 2-methyl-4-(2,2,2-trifluoroethyl) aniline and 2-methyl-4-(2,2,2-trifluoroethyl)cyclohexan-1-amine according to General Procedure E to afford the desired product 8-chloro-N-(2-methyl-4-(2,2,2-trifluoroethyl)phenyl)-5,6-dihydrobenzo[f]imidazo[1,5-d][1,4]oxazepine-10-carboxamide (3.0 mg, 0.0067 mmol, 2% yield) as a white solid. ¹H NMR (400 MHz, DMSO-D6) δ 10.00 (s, 1H), 8.36 (d, J=2.2 Hz, 1H), 7.93 (d, J=2.1 Hz, 1H), 7.85 (d, J=1.1 Hz, 1H), 7.68 (d, J=1.1 Hz, 1H), 7.34 (d, J=8.1 Hz, 1H), 7.27 (s, 1H), 7.22 (d, J=8.1 Hz, 1H), 4.63-4.51 (m, 4H), 3.63 (q, J=11.6 Hz, 2H), 2.24 (s, 3H). ¹⁹F NMR (376 MHz, DMSO-D6) δ-64.27 (t, J=11.7 Hz, CF₃). UPLC-MS (Basic 6 min): rt=3.11 min, m/z (ES+) 436.1, 438.1

### 8-Chloro-N-(2-methyl-4-(2,2,2-trifluoroethyl)cyclohexyl)-5,6-dihydrobenzo[f]imidazo[1,5-d][1,4]oxazepine-10-carboxamide 183A

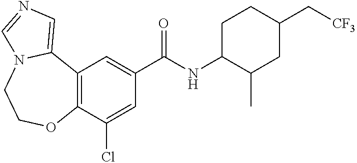 [SMILES: CC1CC(CC(F)(F)F)CCC1NC(=O)c1cc(Cl)c2c(c1)-c1cncn1CCO2]

Also isolated from the same reaction mixture was the other desired product 8-chloro-N-(2-methyl-4-(2,2,2-trifluoroethyl)cyclohexyl)-5,6-dihydrobenzo[f]imidazo[1,5-d][1,4]oxazepine-10-carboxamide (7.0 mg, 0.0152 mmol, 5% yield) as a white solid. ¹H NMR (400 MHz, DMSO-D6) δ 8.30-8.16 (m, 2H), 7.88-7.81 (m, 2H), 7.65-7.60 (m, 1H), 4.59-4.48 (m, 4H), 3.93 (d, J=4.6 Hz, 1H), 2.46-2.06 (m, 3H), 1.92-1.46 (m, 5H), 1.44-1.10 (m, 2H), 1.03-0.82 (m, 3H). ¹⁹F NMR (376 MHz, DMSO-D6) δ−61.45-−61.58 (m, CF₃, major), −62.45 (t, J=12.1 Hz, CF₃, minor). UPLC-MS (Basic 6 min): rt=3.31 min, m/z (ES+) 442.2, 444.2 [M+H]⁺.

### 8-Chloro-N-(cis-2-methyl-cis-4-(2,2,2-trifluoroethyl)cyclohexyl)-5,6-dihydrobenzo[f]imidazo[1,5-d][1,4]oxazepine-10-carboxamide 184A

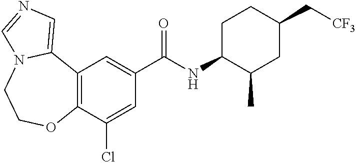 [SMILES: C[C@@H]1C[C@H](CC(F)(F)F)CC[C@@H]1NC(=O)c1cc(Cl)c2c(c1)-c1cncn1CCO2]

Also isolated from the same reaction mixture was the other desired product 8-chloro-N-(cis-2-methyl-cis-4-(2,2,2-trifluoroethyl)cyclohexyl)-5,6-dihydrobenzo[f]imidazo[1,5-d][1,4]oxazepine-10-carboxamide (5.0 mg, 0.0110 mmol, 4% yield) as a white solid. ¹H NMR (400 MHz, DMSO-D6) δ 8.08 (d, J=2.1 Hz, 1H), 7.87-7.77 (m, 3H), 7.58 (d, J=1.1 Hz, 1H), 4.61-4.54 (m, 2H), 4.54-4.49 (m, 2H), 4.24-4.18 (m, 1H), 2.19 (qd, J=12.2, 6.7 Hz, 2H), 1.83-1.31 (m, 8H), 0.85 (d, J=6.8 Hz, 3H). ¹⁹F NMR (376 MHz, DMSO-D6) δ-61.36 (t, J=12.2 Hz, CF₃). UPLC-MS (Basic 6 min): rt=3.37 min, m/z (ES+) 442.2, 444.2 [M+H]⁺. The absolute stereochemistry of 184A is drawn arbitrarily in the depiction above and is intended to reflect the relative stereochemistry of the compound.

### 8-Fluoro-N-(2-methyl-4-(2,2,2-trifluoroethyl)cyclohexyl)-5,6-dihydrobenzo[f]imidazo[1,5-d][1,4]oxazepine-10-carboxamide 185A

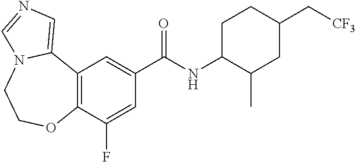 [SMILES: CC1CC(CC(F)(F)F)CCC1NC(=O)c1cc(F)c2c(c1)-c1cncn1CCO2]

Prepared from lithium 8-fluoro-5,6-dihydrobenzo[f]imidazo[1,5-d][1,4]oxazepine-10-carboxylate and 2-methyl-4-(2,2,2-trifluoroethyl)cyclohexan-1-amine according to General Procedure E to afford the desired product 8-fluoro-N-(2-methyl-4-(2,2,2-trifluoroethyl)cyclohexyl)-5,6-dihydrobenzo[f]imidazo[1,5-d][1,4]oxazepine-10-carboxamide (19 mg, 0.0446 mmol, 11% yield) as a white solid. ¹H NMR (400 MHz, DMSO-D6) δ 8.19 (dt, J=17.1, 8.9 Hz, 1H), 8.07 (dt, J=5.4, 1.8 Hz, 1H), 7.82 (d, J=1.9 Hz, 1H), 7.64 (dd, J=3.3, 1.1 Hz, 1H), 7.59 (ddt, J=13.5, 5.6, 2.8 Hz, 1H), 4.53 (d, J=1.8 Hz, 4H), 4.01-3.42 (m, 1H), 2.89, 2.44-2.35 and 2.19 (3×m, 2H), 1.90-1.78 (m, 2H), 1.78-1.50 (m, 3H), 1.50-1.30 (m, 1H), 1.29-0.80 (m, 5H). ¹⁹F NMR (376 MHz, DMSO-D6) δ-61.39--62.55 (m, CF₃), −130.73-−131.13 (m, F). UPLC-MS (Basic 6 min): rt=3.11 min, m/z (ES+) 426.3 [M+H]⁺.

### 8-Fluoro-N-(cis-2-methyl-cis-4-(2,2,2-trifluoroethyl)cyclohexyl)-5,6-dihydrobenzo[f]imidazo[1,5-d][1,4]oxazepine-10-carboxamide 186A

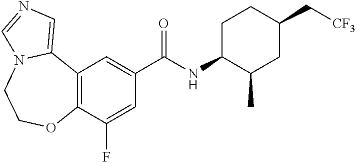 [SMILES: C[C@@H]1C[C@H](CC(F)(F)F)CC[C@@H]1NC(=O)c1cc(F)c2c(c1)-c1cncn1CCO2]

Also isolated from the same reaction mixture was the other desired product 8-fluoro-N-(cis-2-methyl-cis-4-(2,2,2-trifluoroethyl)cyclohexyl)-5,6-dihydrobenzo[f]imidazo[1,5-d][1,4]oxazepine-10-carboxamide (28 mg, 0.0657 mmol, 17% yield) as a white solid. ¹H NMR (400 MHz, DMSO-D6) δ 7.97 (t, J=1.8 Hz, 1H), 7.83 (d, J=1.1 Hz, 1H), 7.71 (d, J=9.0 Hz, 1H), 7.61 (dd, J=11.2, 2.1 Hz, 1H), 7.58 (d, J=1.1 Hz, 1H), 4.54 (s, 4H), 4.21 (dd, J=8.7, 3.9 Hz, 1H), 2.19 (qd, J=12.2, 6.7 Hz, 2H), 1.82-1.64 (m, 3H), 1.62-1.32 (m, 5H), 0.84 (d, J=6.8 Hz, 3H). ¹⁹F NMR (376 MHz, DMSO-D6) δ-61.36 (t, J=12.2 Hz, CF₃), −131.10 (dd, J=11.2, 1.6 Hz, F). UPLC-MS (Basic 6 min): rt=3.19 min, m/z (ES+) 426.3 [M+H]⁺. The absolute stereochemistry of 186A is drawn arbitrarily in the depiction above and is intended to reflect the relative stereochemistry of the compound.

### 8,9-Difluoro-N-((1r,4r)-4-(2,2,2-trifluoroethoxy)cyclohexyl)-5,6-dihydrobenzo[f]imidazo[1,5-d][1,4]oxazepine-10-carboxamide 187A

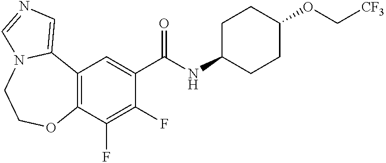 [SMILES: O=C(N[C@H]1CC[C@H](OCC(F)(F)F)CC1)c1cc2c(c(F)c1F)OCCn1cncc1-2]

Prepared from lithium 8,9-difluoro-5,6-dihydrobenzo[f]imidazo[1,5-d][1,4]oxazepine-10-carboxylate and 4-(2,2,2-trifluoroethoxy)cyclohexanamine according to General Procedure E to afford the desired product 8,9-difluoro-N-((1r,4r)-4-(2,2,2-trifluoroethoxy)cyclohexyl)-5,6-dihydrobenzo[f]imidazo[1,5-d][1,4]oxazepine-10-carboxamide (11 mg, 0.0247 mmol, 3% yield) as a white solid. ¹H NMR (400 MHz, DMSO-D6) δ 8.29 (d, J=7.7 Hz, 1H), 7.80 (d, J=1.1 Hz, 1H), 7.71 (dd, J=7.4, 2.2 Hz, 1H), 7.56 (d, J=1.1 Hz, 1H), 4.60-4.49 (m, 4H), 4.06 (q, J=9.4 Hz, 2H), 3.80-3.66 (m, 1H), 3.49-3.39 (m, 1H), 2.09-1.95 (m, 2H), 1.95-1.82 (m, 2H), 1.40-1.25 (m, 4H). ¹⁹F NMR (376 MHz, DMSO-D6) δ-73.09 (t, J=9.4 Hz, CF₃), −140.22 (dd, J=22.3, 7.4 Hz, F), −155.23--155.51 (m, F). UPLC-MS (Basic 6 min): rt=2.88 min, m/z (ES+) 446.2 [M+H]⁺.

### 8,9-Difluoro-N-((1s,4s)-4-(2,2,2-trifluoroethoxy)cyclohexyl)-5,6-dihydrobenzo[f]imidazo[1,5-d][1,4]oxazepine-10-carboxamide 188A

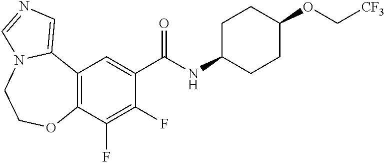 [SMILES: O=C(N[C@H]1CC[C@@H](OCC(F)(F)F)CC1)c1cc2c(c(F)c1F)OCCn1cncc1-2]

Also isolated from the same reaction mixture was the other desired product 8,9-difluoro-N-((1s,4s)-4-(2,2,2-trifluoroethoxy)cyclohexyl)-5,6-dihydrobenzo[f]imidazo[1,5-d][1,4]oxazepine-10-carboxamide (50 mg, 0.112 mmol, 11% yield) as a white solid. ¹H NMR (400 MHz, DMSO-D6) δ 8.34 (d, J=7.7 Hz, 1H), 7.79 (d, J=1.0 Hz, 1H), 7.71 (dd, J=7.4, 2.3 Hz, 1H), 7.57 (d, J=1.1 Hz, 1H), 4.60-4.49 (m, 4H), 4.04 (q, J=9.4 Hz, 2H), 3.91-3.74 (m, 1H), 3.70-3.60 (m, 1H), 1.92-1.77 (m, 2H), 1.71-1.50 (m, 6H). ¹⁹F NMR (376 MHz, DMSO-D6) 8-72.97 (t, J=9.5 Hz, CF₃), −140.08 (dd, J=22.2, 7.4 Hz, F), −155.48 (dd, J=22.3, 2.4 Hz, F). UPLC-MS (Basic 6 min): rt=2.94 min, m/z (ES+) 446.2 [M+H]⁺.

### 8,9-Dimethyl-N-(4-(2,2,2-trifluoroethoxy)cyclohexyl)-5,6-dihydrobenzo[f]imidazo[1,5-d][1,4]oxazepine-10-carboxamide 189A

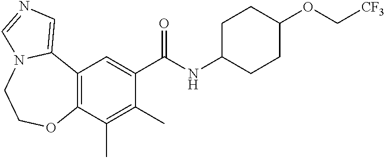 [SMILES: Cc1c(C(=O)NC2CCC(OCC(F)(F)F)CC2)cc2c(c1C)OCCn1cncc1-2]

Prepared from lithium 8,9-dimethyl-5,6-dihydrobenzo[f]imidazo[1,5-d][1,4]oxazepine-10-carboxylate and 4-(2,2,2-trifluoroethoxy)cyclohexanamine according to General Procedure E to afford the desired product 8,9-dimethyl-N-(4-(2,2,2-trifluoroethoxy)cyclohexyl)-5,6-dihydrobenzo[f]imidazo[1,5-d][1,4]oxazepine-10-carboxamide (24 mg, 0.0548 mmol, 6% yield) as a white solid. ¹H NMR (400 MHz, DMSO-D6) δ 8.21-8.09 (m, 1H), 7.75 (d, J=1.1 Hz, 1H), 7.44-7.37 (m, 2H), 4.45-4.35 (m, 4H), 4.04 (dq, J=13.7, 9.4 Hz, 2H), 3.88-3.68 (m, 1H), 3.65 (d, J=5.2 Hz, 1H), 2.24-2.18 (m, 3H), 2.18-2.13 (m, 3H), 2.07-1.78 (m, 2H), 1.78-1.10 (m, 6H). ¹⁹F NMR (376 MHz, DMSO-D6) δ-72.97 (t, J=9.4 Hz, CF₃ (cis)), −73.09 (t, J=9.4 Hz, CF₃ (trans)). UPLC-MS (Basic 6 min): rt=2.85 min, m/z (ES+) 438.3 (trans) [M+H]+, rt =2.89 min, m/z (ES+) 438.3 (cis) [M+H]⁺.

### 8-Chloro-N-(4,4-difluorocyclohexyl)-9-methyl-5,6-dihydrobenzo[f]imidazo[1,5-d][1,4]oxazepine-10-carboxamide 190A

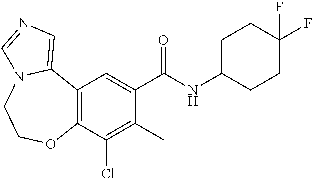 [SMILES: Cc1c(C(=O)NC2CCC(F)(F)CC2)cc2c(c1Cl)OCCn1cncc1-2]

Prepared from lithium 8-chloro-9-methyl-5,6-dihydrobenzo[f]imidazo[1,5-d][1,4]oxazepine-10-carboxylate and 4,4-difluorocyclohexanamine hydrochloride according to General Procedure E to afford the desired product 8-chloro-N-(4,4-difluorocyclohexyl)-9-methyl-5,6-dihydrobenzo[f]imidazo[1,5-d][1,4]oxazepine-10-carboxamide (53 mg, 0.131 mmol, 39% yield) as a white solid. ¹H NMR (400 MHz, DMSO-D6) δ 8.37 (d, J=7.7 Hz, 1H), 7.80 (d, J=1.1 Hz, 1H), 7.64 (s, 1H), 7.56 (d, J=1.1 Hz, 1H), 4.54-4.46 (m, 4H), 3.98-3.91 (m, 1H), 2.32 (s, 3H), 2.09-1.97 (m, 3H), 1.96-1.88 (m, 3H), 1.65-1.55 (m, 2H). ¹⁹F NMR (376 MHz, DMSO-D6) δ-92.88 (d, J=232.5 Hz, F), −97.72 (d, J=233.0 Hz, F). UPLC-MS (Basic 6 min): rt =2.63 min, m/z (ES+) 396.2, 398.2 [M+H]⁺.

### 8-Chloro-N-((1r,4r)-4-(2-methoxyethoxy)cyclohexyl)-9-methyl-5,6-dihydrobenzo[f]imidazo[1,5-d][1,4]oxazepine-10-carboxamide 191A

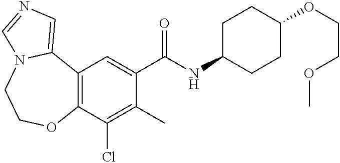 [SMILES: COCCO[C@H]1CC[C@H](NC(=O)c2cc3c(c(Cl)c2C)OCCn2cncc2-3)CC1]

Prepared from lithium 8-chloro-9-methyl-5,6-dihydrobenzo[f]imidazo[1,5-d][1,4]oxazepine-10-carboxylate and trans-4-(2-methoxyethoxy)cyclohexanamine according to General Procedure E to afford the desired product 8-chloro-N-((1r,4r)-4-(2-methoxyethoxy)cyclohexyl)-9-methyl-5,6-dihydrobenzo[f]imidazo[1,5-d][1,4]oxazepine-10-carboxamide (51 mg, 0.119 mmol, 35% yield) as a white solid. ¹H NMR (400 MHz, DMSO-D6) δ 8.25 (d, J=7.8 Hz, 1H), 7.80 (d, J=1.1 Hz, 1H), 7.60 (s, 1H), 7.54 (d, J=1.1 Hz, 1H), 4.54-4.43 (m, 4H), 3.75-3.63 (m, 1H), 3.56-3.49 (m, 2H), 3.46-3.37 (m, 2H), 3.27-3.16 (m, 4H), 2.31 (s, 3H), 2.03-1.95 (m, 2H), 1.94-1.86 (m, 2H), 1.37-1.16 (m, 4H). UPLC-MS (Basic 6 min): rt=2.36 min, m/z (ES+) 434.3, 436.2 [M+H]⁺.

### Example 4: NAD⁺ Cydase Activity Assay with Human or Mouse Recombinant CD38 Protein

The following assay was used to determine the ability of compounds of the invention to inhibit the hydrolysis of NAD⁺ by the human or mouse enzyme, CD38.

### Final Assay Conditions for Assay Protocol

Reaction buffer: 40 mM HEPES/NaOH pH 7.5, 250 mM sucrose, 0.01% Tween

Final concentrations: 0.6 nM mouse CD38 (4947-AC, Bio-Techne GmbH) 1.9 nM human CD38 (AVI2404, Bio-Techne GmbH)

Substrate: 15.8 M Nicotinamide 1,N6-ethenoadenine dinucleotide (&-NAD)

Read-out: Fluorescence intensity (extinction 300 nm/emission 410 nm)

### Assay Protocol

Required reaction buffer, required CD38 concentration in reaction buffer and required e-NAD concentration in reaction buffer were prepared. Thereafter, 8 μL of CD38 in reaction buffer was added to High Control and compound concentration wells in 384 well plate (Corning #4514, 384 well, nonbinding surface, low volume, round bottom) and 8 μL of reaction buffer to Low Control. Compound dilutions were transferred via pintool technology that transfers 42.5 nL compound dilutions (12-point dose-response with factor three dilutions in 100% DMSO) well-to-well. Plate was subsequently shaken for 15 s at 1450 rpm and sealed for incubation for 30 min at room temperature. Thereafter, 2 μL of e-NAD in reaction buffer were added with Integra Voyager. After shaking for 15 s at 1450 rpm, plate was sealed, and assay signal were measured continuously with PHERAstar® fluorescence intensity module (extinction 300 nm/emission 410 nm).

### Data Analysis

Dose-response curves were constructed using twelve-pointfactor three dilutions in triplicates per plate. High control (full enzyme activity) was set to assay signal of CD38 and e-NAD in presence of vehicle (DMSO) and Low control (full enzyme inhibition) was set to assay signal of e-NAD and vehicle (DMSO) in absence of CD38. For data analysis, fluorescent signal of traces corresponding to each compound concentration and High and Low control were monitored over time. Each run, High Control (full enzyme activity) was fitted to Linear equation via GraphPad Prism to determine CD38 activity from the slope and determine the time interval of linear phase of reaction to account for minor fluctuations. Thereafter, slopes of all compound concentrations were determined via fitting to same linear equation (GraphPad Prism) over the same time interval as determined for High control. Percentage inhibition was calculated via normalization to High and Low Control via the equation: %−inhibition=100*(1-((Sample-Low control)/(High control−Low control))) Half maximal inhibitory concentration (IC50) values were yielded from the inflection point of the corresponding curve by fitting of resulting percentage inhibition values to standard equation (4 Parameter Logistic Model or Sigmoidal Dose-Response Model) via Excel Fit. Unless otherwise stated, fitting of curves were fixed to 100%-inhibition and unlocked for 0%-inhibition.

Compounds of the invention are potent inhibitors of CD38 as demonstrated by the results shown in the Table 1. For Human CD38, compounds having an IC₅₀ (nM) of less than 30.0 nM as ++++, 30.0 nM to 100 nM as +++, 100 nM to 1000 nM as ++, and greater than 1000 nM as +. For Mouse CD38, compounds having an IC50 (nM) of less than 1.0 nM as ++++, 1.0 nM to 10.0 nM as +++, 10.0 nM to 100 nM as ++, and greater than 100.0 nM as +.

TABLE 1
CD38 IC₅₀ Values

| | Human CD38 | Mouse CD38 |
|---|---|---|
| Compound number | IC50 | IC50 |
| 1A | ++++ | ++++ |
| 2A | ++++ | +++ |
| 3A | ++++ | +++ |
| 4A | ++++ | +++ |
| 5A | ++++ | ++++ |
| 6A | ++++ | +++ |
| 7A | ++++ | +++ |
| 8A | ++++ | +++ |
| 9A | ++++ | +++ |
| 10A | ++++ | +++ |
| 11A | + | + |
| 12A | + | + |
| 13A | + | + |
| 14A | + | + |
| 15A | + | + |
| 16A | + | + |
| 17A | ++++ | ++++ |
| 18A | ++++ | ++++ |
| 19A | ++++ | ++++ |
| 20A | ++++ | ++++ |
| 21A | ++++ | ++++ |
| 22A | ++++ | +++ |
| 23A | +++ | ++ |
| 24A | +++ | ++ |
| 25A | ++++ | +++ |
| 26A | ++ | ++ |
| 27A | ++ | +++ |
| 28A | ++++ | +++ |
| 29A | ++++ | +++ |
| 30A | ++++ | +++ |
| 31A | ++++ | +++ |
| 32A | ++++ | +++ |
| 33A | ++++ | +++ |
| 34A | ++++ | +++ |
| 35A | + | + |
| 36A | ++++ | ++++ |
| 37A | ++++ | +++ |
| 38A | + | ++ |
| 39A | ++++ | +++ |
| 40A | ++++ | +++ |
| 41A | ++++ | +++ |
| 42A | ++ | ++ |
| 43A | + | |
| 44A | ++++ | |
| 45A | ++++ | |
| 46A | ++++ | ++++ |
| 47A | ++++ | +++ |
| 48A | ++++ | +++ |
| 49A | ++++ | ++ |
| 50A | +++ | +++ |
| 51A | + | + |
| 52A | ++++ | +++ |
| 53A | ++++ | ++++ |
| 54A | ++++ | +++ |
| 55A | +++ | ++++ |
| 56A | ++++ | +++ |
| 57A | ++++ | ++++ |
| 58A | ++++ | ++++ |
| 59A | ++++ | +++ |
| 60A | ++ | +++ |
| 61A | ++++ | ++++ |
| 62A | ++++ | +++ |
| 63A | ++++ | +++ |
| 64A | ++ | ++ |
| 65A | ++++ | ++++ |
| 66A | ++++ | ++++ |
| 67A | ++ | +++ |
| 68A | ++ | ++ |
| 69A | ++ | ++ |
| 70A | ++ | ++ |
| 71A | ++++ | +++ |
| 72A | ++++ | ++++ |
| 73A | ++++ | ++++ |
| 74A | +++ | +++ |
| 75A | + | + |
| 76A | ++++ | +++ |
| 77A | ++++ | +++ |
| 78A | ++ | + |
| 79A | ++ | ++ |
| 80A | ++++ | +++ |
| 81A | ++++ | +++ |
| 82A | ++++ | +++ |
| 83A | ++ | ++ |
| 84A | ++++ | +++ |
| 85A | ++++ | +++ |
| 86A | ++ | + |
| 87A | ++++ | ++++ |
| 88A | ++++ | ++++ |
| 89A | ++ | + |
| 90A | ++++ | ++++ |
| 91A | +++ | +++ |
| 92A | ++++ | +++ |
| 93A | ++++ | +++ |
| 94A | ++++ | ++++ |
| 95A | ++++ | +++ |
| 96A | ++++ | +++ |
| 97A | ++++ | ++++ |
| 98A | ++++ | +++ |
| 99A | ++++ | ++ |
| 100A | ++++ | +++ |
| 101A | ++++ | ++++ |
| 102A | ++++ | +++ |
| 103A | ++++ | +++ |
| 104A | ++++ | +++ |
| 105A | +++ | +++ |
| 106A | +++ | +++ |
| 107A | +++ | +++ |
| 108A | ++++ | ++++ |
| 109A | ++++ | +++ |
| 110A | +++ | +++ |
| 111A | ++ | + |
| 112A | ++++ | ++++ |
| 113A | ++++ | +++ |
| 114A | ++++ | ++++ |
| 115A | ++++ | ++++ |
| 116A | ++++ | ++++ |
| 117A | +++ | +++ |
| 118A | + | + |
| 119A | ++++ | +++ |
| 120A | ++++ | +++ |
| 121A | + | + |
| 122A | ++++ | +++ |
| 123A | ++ | ++ |
| 124A | ++++ | ++ |
| 125A | ++++ | +++ |
| 126A | +++ | +++ |
| 127A | ++++ | +++ |
| 128A | ++ | ++ |
| 129A | ++++ | +++ |
| 130A | +++ | +++ |
| 131A | ++++ | ++++ |
| 132A | ++++ | +++ |
| 133A | +++ | +++ |
| 134A | ++++ | ++++ |
| 135A | ++++ | ++++ |
| 136A | +++ | +++ |
| 137A | ++++ | ++++ |
| 138A | ++++ | +++ |
| 139A | ++++ | ++++ |
| 140A | ++++ | ++++ |
| 141A | ++++ | +++ |
| 142A | ++++ | +++ |
| 143A | ++++ | ++++ |
| 144A | ++++ | +++ |
| 145A | ++++ | +++ |
| 146A | +++ | ++ |
| 147A | ++++ | +++ |
| 148A | ++++ | +++ |
| 149A | ++ | ++ |
| 150A | ++ | + |
| 151A | ++++ | +++ |
| 152A | ++++ | +++ |
| 153A | ++ | ++ |
| 154A | ++++ | ++++ |
| 155A | ++++ | ++++ |
| 156A | +++ | ++ |
| 157A | ++++ | ++++ |
| 158A | +++ | +++ |
| 159A | ++++ | ++++ |
| 160A | +++ | +++ |
| 161A | ++++ | +++ |
| 162A | +++ | ++ |
| 163A | ++++ | +++ |
| 164A | ++++ | +++ |
| 165A | ++++ | +++ |
| 166A | ++++ | +++ |
| 167A | ++ | ++ |
| 168A | ++ | + |
| 169A | +++ | ++ |
| 170A | +++ | +++ |
| 171A | + | + |
| 172A | ++++ | +++ |
| 173A | ++ | ++ |
| 174A | ++++ | ++++ |
| 175A | ++++ | +++ |
| 176A | ++++ | +++ |
| 177A | ++++ | +++ |
| 178A | ++++ | +++ |
| 179A | ++ | |
| 180A | ++ | |
| 181A | ++++ | |
| 182A | +++ | |
| 183A | ++++ | |
| 184A | ++ | |
| 185A | ++++ | |
| 186A | +++ | |
| 187A | ++++ | |
| 188A | ++ | |
| 189A | ++ | |
| 190A | +++ | |
| 191A | +++ | |

1.-60. (canceled)

61. A compound of Formula (II): 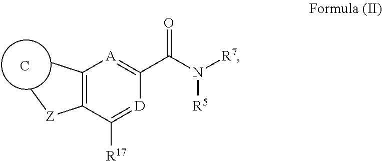
or a pharmaceutically acceptable salt or solvate thereof; wherein
Z is selected from 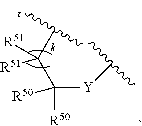
wherein t represents the point of connection between Z and \textcircled{C};
Y is selected from —O—, —NR⁹—, —S—, and —SO₂—;
each R⁵⁰ is independently selected from hydrogen, halogen, and C₁-C₆ alkyl; or both R⁵⁰ come together to form 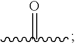 [SMILES: *=O]
each R⁵¹ is independently selected from hydrogen, halogen, and C₁-C₆ alkyl;
k is selected from 1 and 2;
A is selected from N and CR¹⁸;
\textcircled{C} is selected from an optionally substituted imidazole, wherein the imidazole is optionally substituted with one or more R¹⁴;
D is selected from N and CR¹⁹;
R⁵ is selected from hydrogen and C₁₋₆ alkyl;
R⁷ is selected from hydrogen; and C₁₋₁₀ alkyl, C₂₋₁₀ alkenyl, C₂₋₁₀ alkynyl, C₃-C₁₄ carbocycle, and 5- to 15-membered heterocycle, each of which are optionally substituted with one or more R⁸;
each R⁸ is independently selected from halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, ═O, ═N(R²⁰), ═NO(R²⁰), —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃-C₁₂ carbocycle, and 4- to 12-membered heterocycle, wherein the C₁₋₆ alkyl is optionally substituted with one or more R⁸*, and wherein the C₃-C₁₂ carbocycle and 4- to 12-membered heterocycle are each optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
each R⁸* is independently selected from 4- to 12-membered heterocycle, wherein the 4- to 12-membered heterocycle is optionally substituted with one or more substituents selected from halogen, —OH, —CN, —NO₂, —NH₂, oxo, C₁₋₁₀ alkyl, —C₁₋₁₀ haloalkyl, —O—C₁₋₁₀ alkyl, C₁-C₆ hydroxyalkyl, C₁-C₆ aminoalkyl, C₁-C₆ cyanoalkyl, and C₁-C₆ alkoxyalkyl;
R⁹ is selected from hydrogen, C₁-C₆ alkyl, C₁-C₆ haloalkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₃₋₆ carbocycle, and 4- to 6-membered heterocycle, wherein the C₃₋₆ carbocycle and 4- to 6-membered heterocycle are each optionally substituted with one or more substituents independently selected from halogen, —OH, —CN, —NO₂, —NH₂, oxo, C₁₋₁₀ alkyl, C₁₋₁₀ haloalkyl, —O—C₁₋₁₀ alkyl, C₁-C₆ hydroxyalkyl, C₁-C₆ aminoalkyl, C₁-C₆ cyanoalkyl, C₁-C₆ alkoxyalkyl, C₁-C₆ alkyl-N(R²⁰)₂, C₂₋₁₀ alkenyl, and C₂₋₁₀ alkynyl;
each R¹⁴ is independently selected from halogen, —OH, —CN, —NO₂, —NH₂, —NHC₁₋₁₀ alkyl, —N(C₁₋₁₀ alkyl)₂, C₁₋₁₀ alkyl, C₁₋₁₀ haloalkyl, —O—C₁₋₁₀ alkyl, C₂₋₁₀ alkenyl, and C₂₋₁₀ alkynyl;
R¹⁷ is selected from hydrogen, halogen, —N(R²⁰)₂, —OR²⁰, —SR²⁰, —S(O)₂(R²⁰), —S)₂N(R²⁰)₂, —S(O)N(R²⁰)₂, —S(O)R²⁰(═NR²⁰), —NR²⁰S(O)₂R²⁰, —C(O)N(R²⁰)₂, —C(═NR²⁰)N(R²⁰)₂, —C(O)NR²⁰OR²⁰, —N(R²⁰)C(O)R²⁰, —N(R²⁰)C(O)N(R²⁰)₂, —N(R²⁰)C(O)OR²⁰, —C(O)R²⁰, —C(O)OR²⁰, —OC(O)R²⁰, —OC(O)N(R²⁰)₂, —NO₂, —CN, C₁₋₆ alkyl-N(R²⁰)₂, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, C₂₋₆ alkynyl, C₃₋₁₂ carbocycle, and 5- to 12-membered heterocycle;
R¹⁸ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl; and
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, —S(O)₂(C₁₋₆ alkyl), C₁₋₁₀ alkyl, C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

62. (canceled)

63. The compound or salt of claim 61, wherein Z is

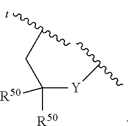

64. The compound or salt of claim 61, wherein Formula (II) is represented by Formula (IIA), Formula (IIB), or Formula (IIC): 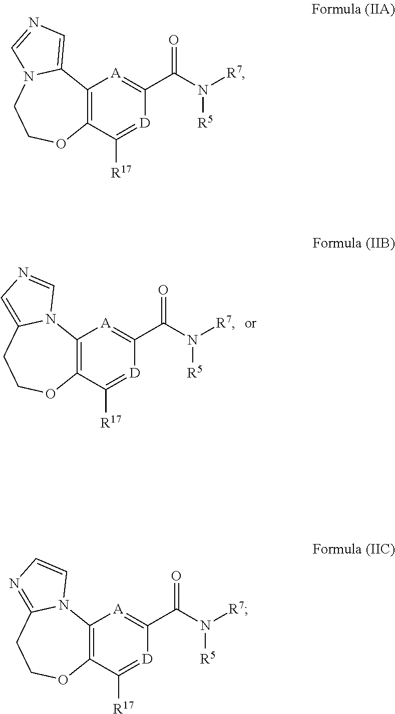
or a pharmaceutically acceptable salt or solvate thereof.

65. (canceled)

66. (canceled)

67. The compound or salt of claim 64, wherein A is selected from N, CF, and CH; D is selected from N, CF, and CH; and R¹⁷ is selected from hydrogen, halogen, —O—C₁₋₆ alkyl, C₁₋₆ haloalkyl, C₁₋₆ alkyl, and C₃₋₆ carbocycle.

68. (canceled)

69. (canceled)

70. (canceled)

71. (canceled)

72. (canceled)

73. The compound or salt of claim 61, wherein Formula (II) is represented by Formula (IID), Formula (IIE), or Formula (IIF): 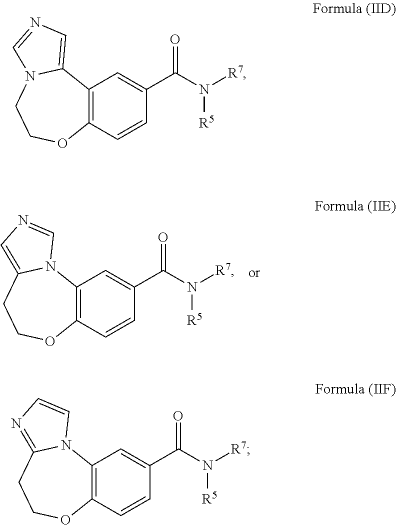
or a pharmaceutically acceptable salt or solvate thereof.

74. (canceled)

75. (canceled)

76. (canceled)

77. (canceled)

78. The compound or salt of claim 61, wherein R⁵ is hydrogen.

79. (canceled)

80. The compound or salt of claim 61, wherein R⁷ is selected from C₆-C₈ carbocycle, which is optionally substituted with one or more R⁸.

81. (canceled)

82. The compound or salt of claim 61, wherein R⁷ is selected from 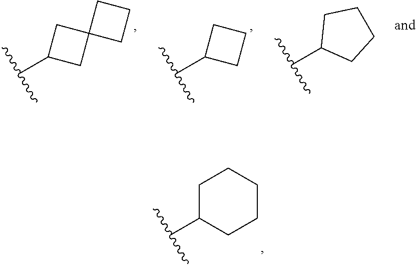 [SMILES: *C1CC2(CCC2)C1 ; *C1CCC1 ; *C1CCCC1 ; *C1CCCCC1]
which is optionally substituted with one or more R⁸.

83. The compound or salt of claim 82, wherein each R⁸ is independently selected from halogen, C₁₋₆ alkyl, C₁₋₆ haloalkyl, C₁₋₆ hydroxyalkyl, —OR²⁰, —N(R²⁰)₂, —NR²⁰S(O)₂R²⁰, —N(R²⁰)C(O)R²⁰, —C(O)N(R²⁰)₂, —S(O)₂(R²⁰), and 4- to 6-membered heterocycle, wherein the 4- to 6-membered heterocycle is optionally substituted with one or more substituents selected from halogen, —OH, —CN, —NH₂, oxo, C₁₋₁₀ alkyl, —C₁₋₁₀ haloalkyl, and —O—C₁₋₁₀ alkyl; and wherein the C₁₋₆ alkyl of R⁸ is optionally substituted with one 4- to 6-membered heterocycle, wherein the 4- to 6-membered heterocycle is optionally substituted with one or more substituents selected from halogen.

84. (canceled)

85. The compound or salt of claim 61, wherein R⁷ is selected from

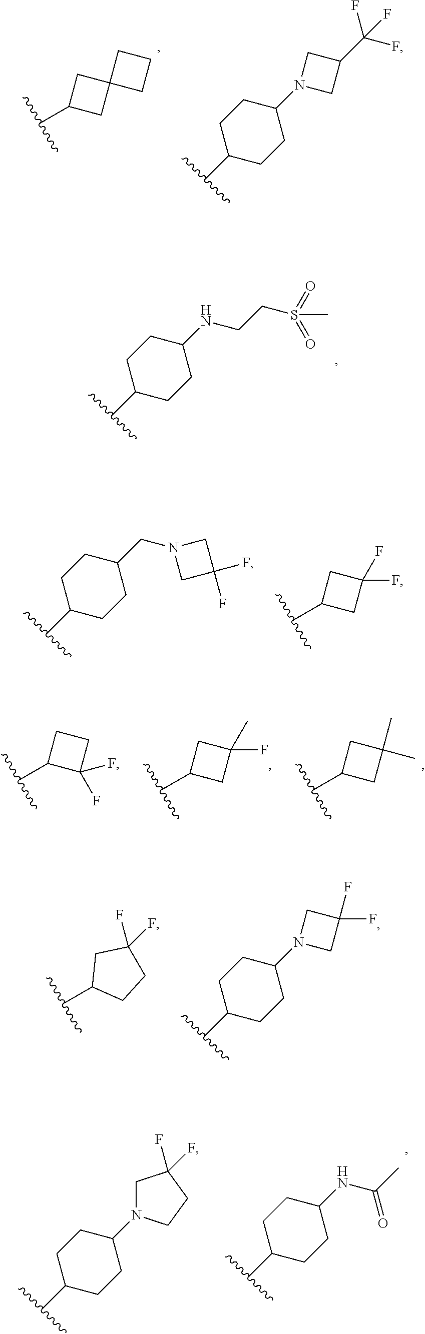

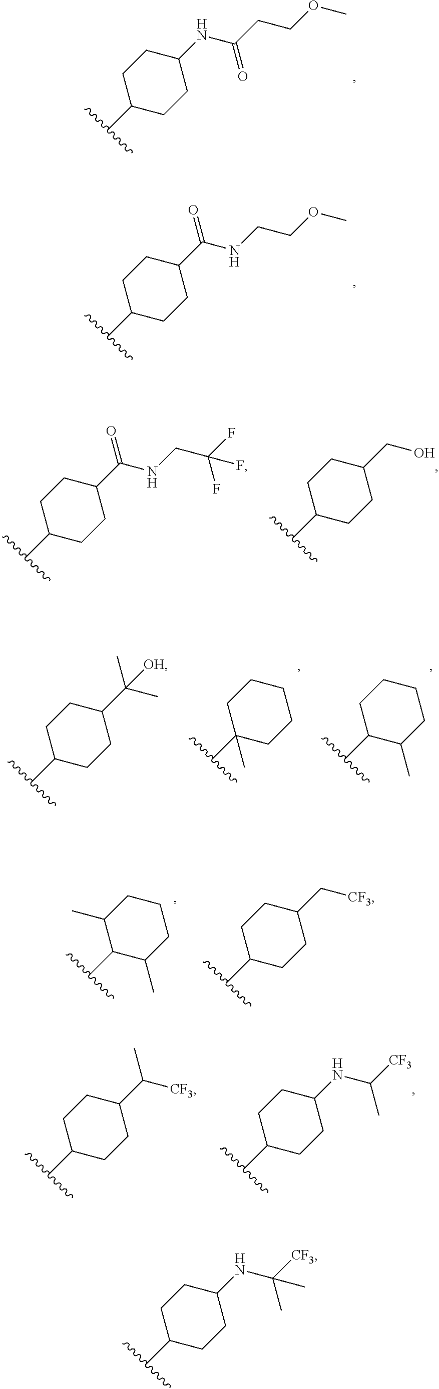

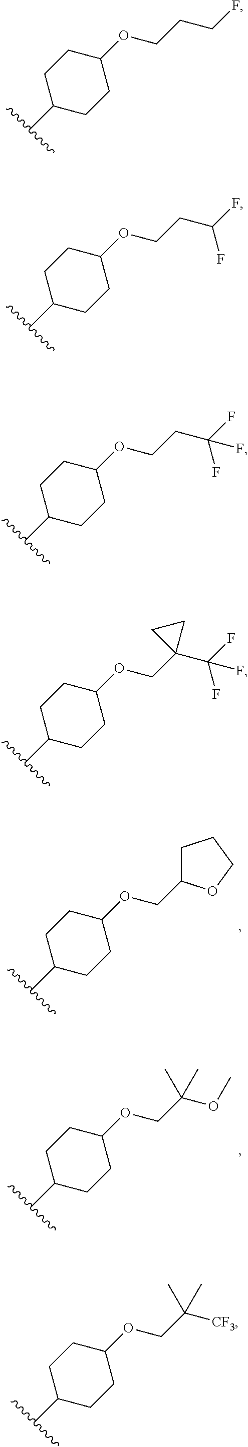

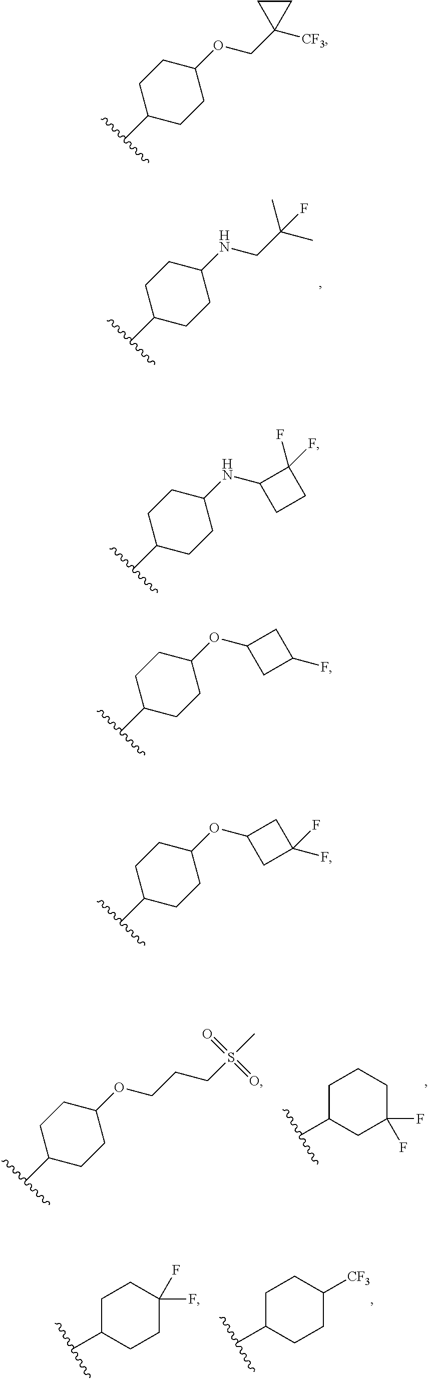

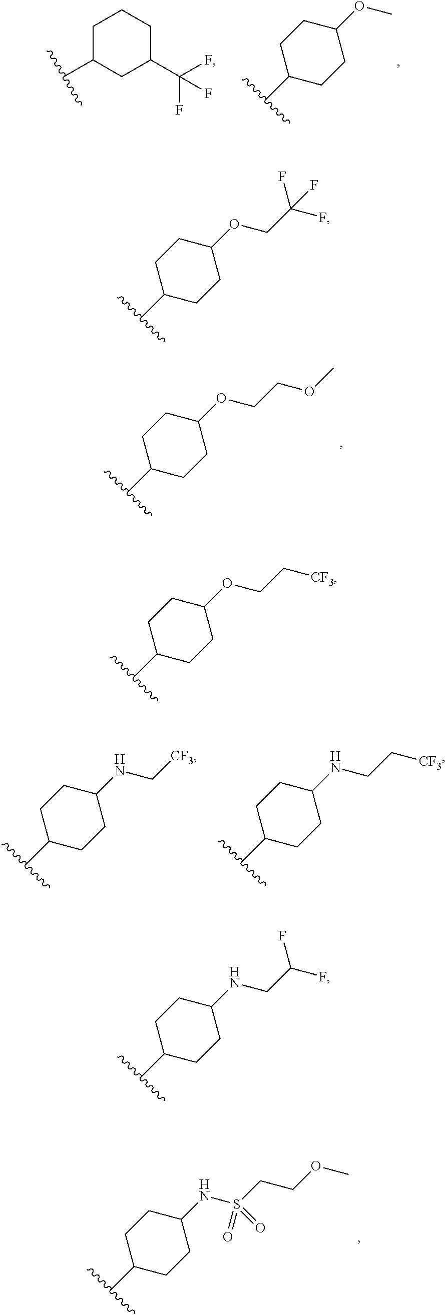

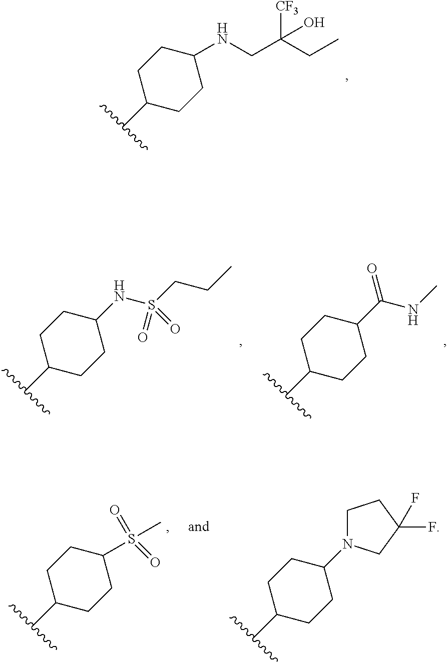 [SMILES: *C1CCC(NCC(O)(CC)C(F)(F)F)CC1 ; *C1CCC(NS(=O)(=O)CCC)CC1 ; *C1CCC(C(=O)NC)CC1 ; *C1CCC(S(C)(=O)=O)CC1 ; *C1CCC(N2CCC(F)(F)C2)CC1]

86. The compound or salt of claim 61, wherein R⁷ is selected from an optionally substituted phenyl, which is optionally substituted with one or more R⁸; or R⁷ is selected from C₆ carbocycle, which is optionally substituted with one or more R⁸.

87. (canceled)

88. (canceled)

89. The compound or salt of claim 61, wherein R⁷ is selected from

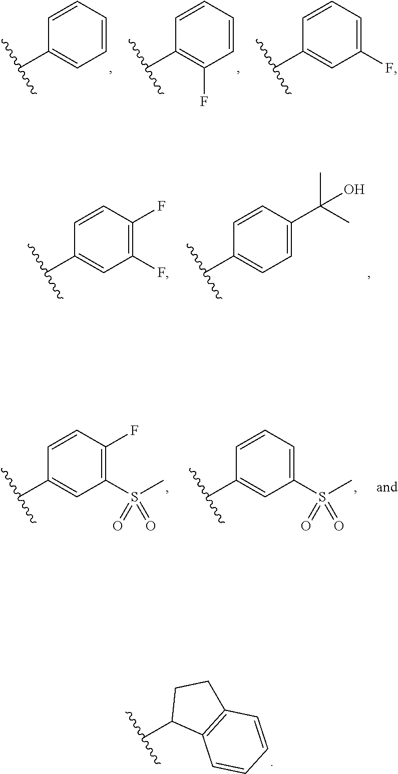

90. (canceled)

91. (canceled)

92. (canceled)

93. (canceled)

94. (canceled)

95. The compound or salt of claim 61, wherein R⁷ is selected from 5- to 10-membered heterocycle, each of which are optionally substituted with one or more R⁸.

96. The compound or salt of claim 61, wherein R⁷ is selected from 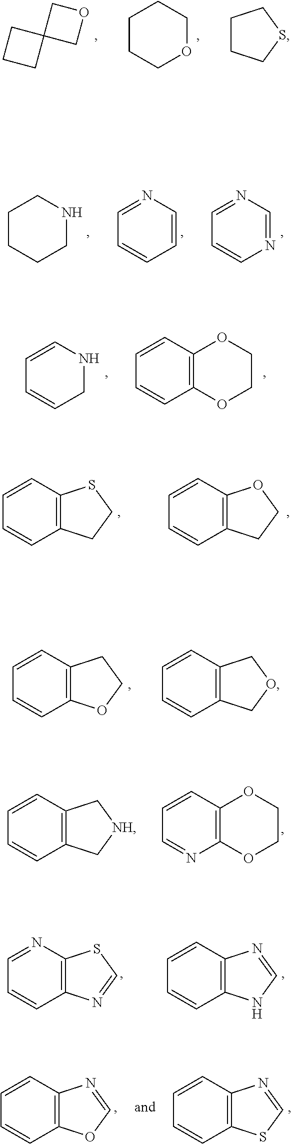
each of which are optionally substituted with one or more R⁸, wherein each R⁸ is independently selected from halogen, —OR²⁰, —N(R²⁰)₂, —S(O)₂(R²⁰), —C(O)OR²⁰═O, C₁₋₆ alkyl, C₁₋₆ haloalkyl, C₁₋₆ hydroxyalkyl, and 4- to 6-membered heterocycle, wherein the 4- to 6-membered heterocycle is optionally substituted with one or more substituents selected from halogen and —C₁₋₃ haloalkyl.

97. (canceled)

98. (canceled)

99. The compound or salt of claim 61, wherein R⁷ is selected from

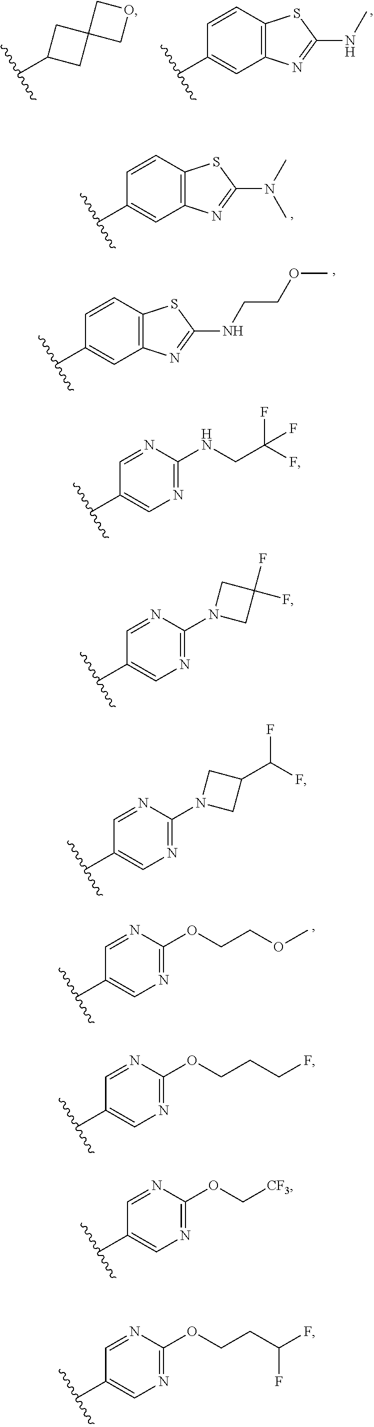

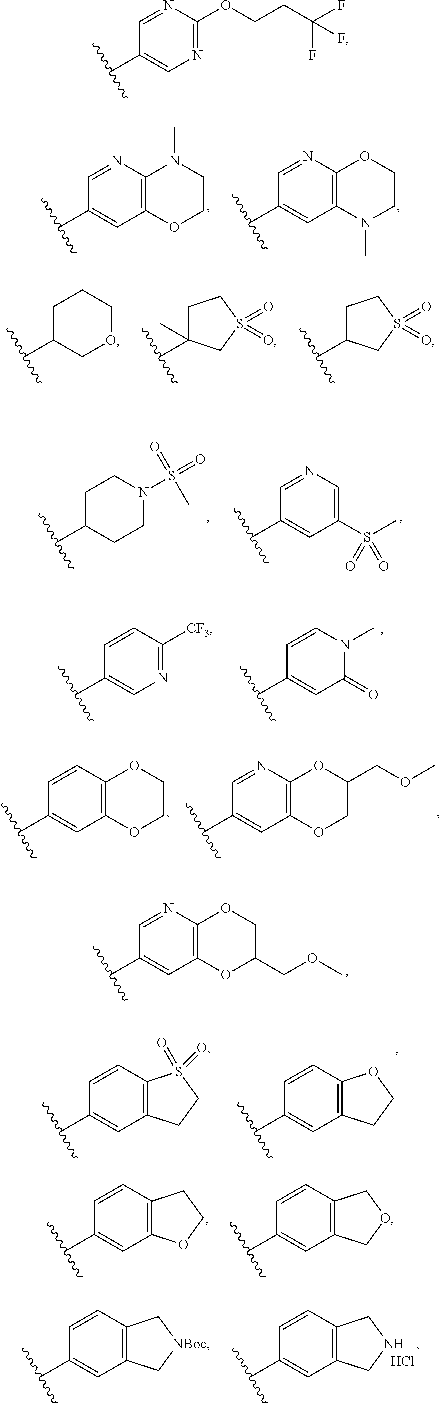

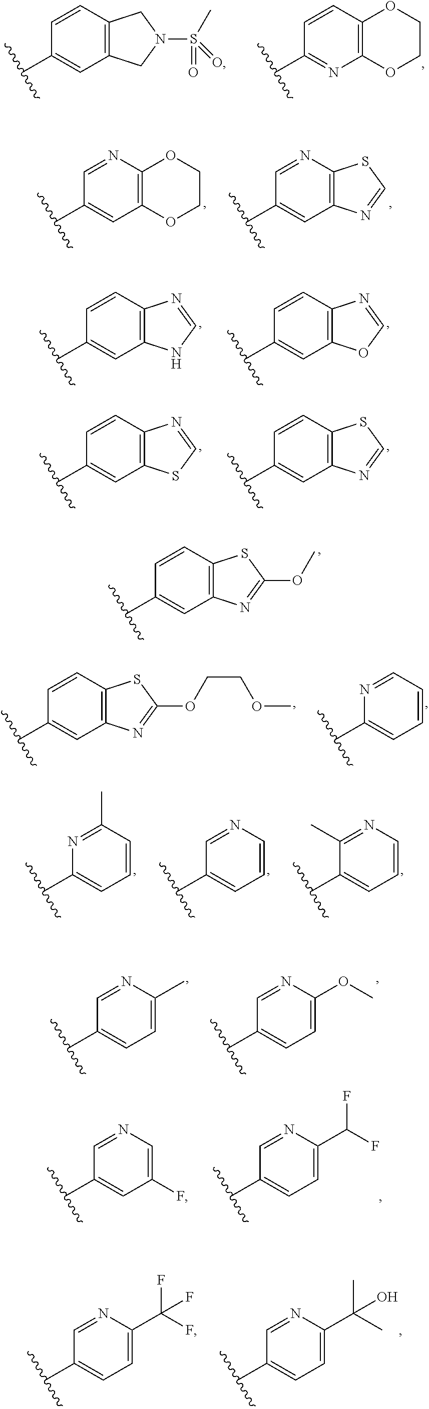

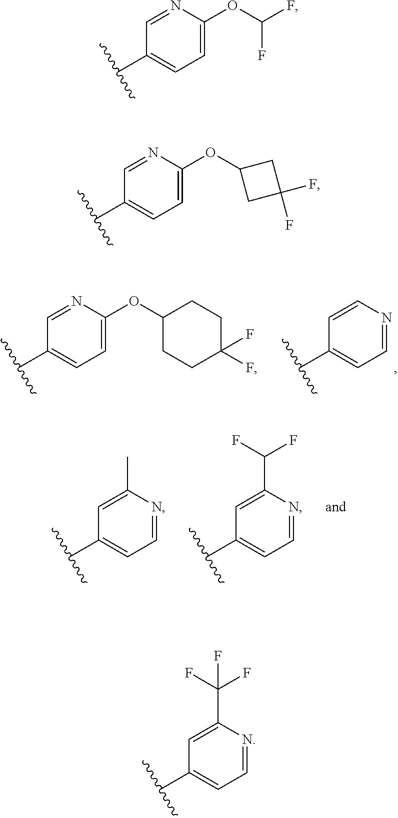

100.-136. (canceled)

137. The compound or salt of claim 61, selected from: 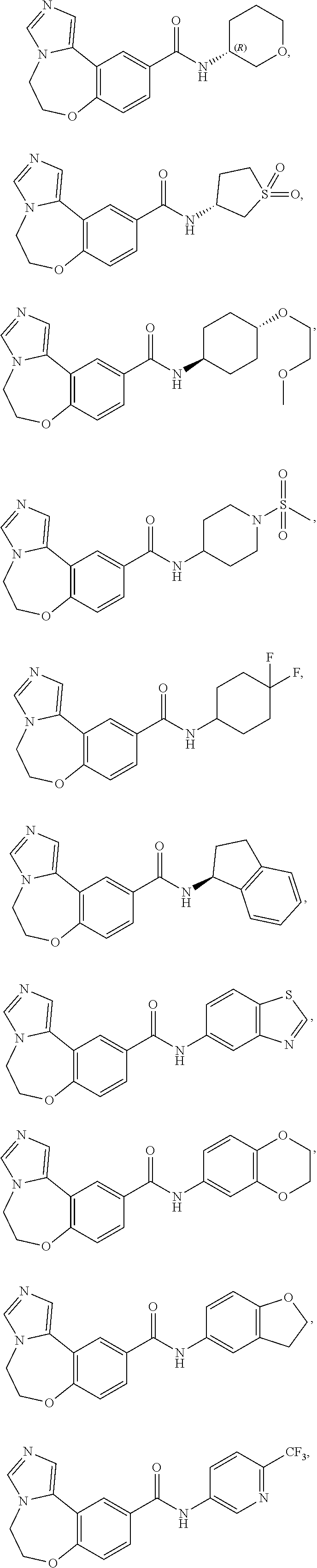 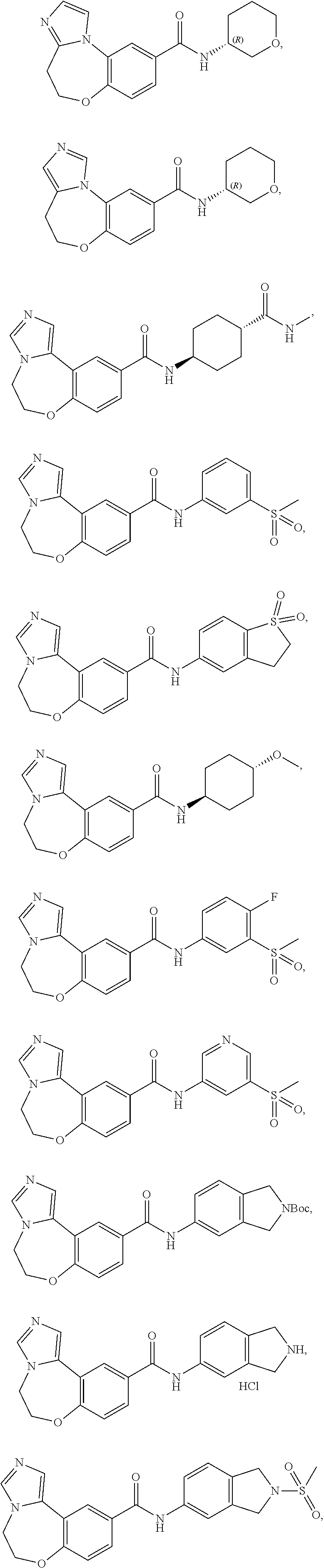 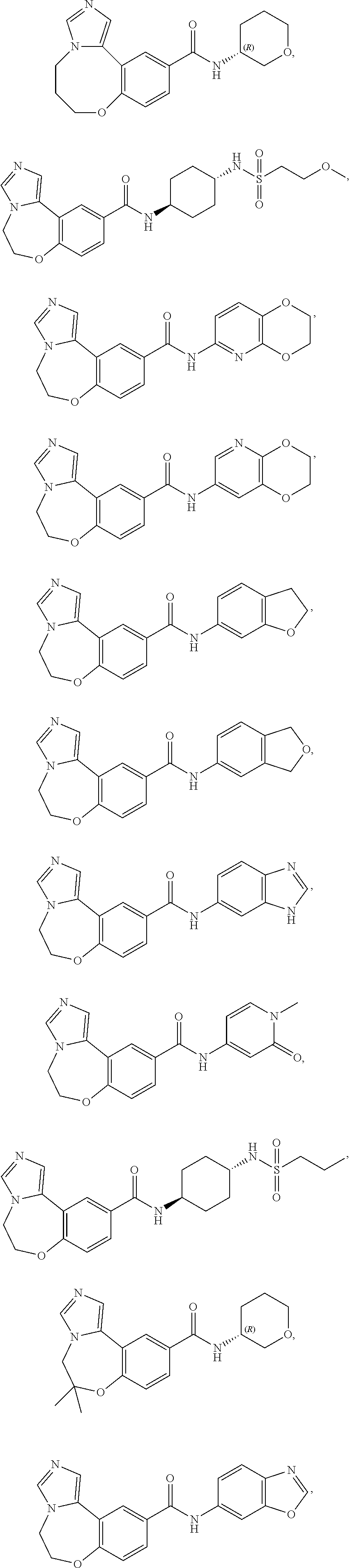 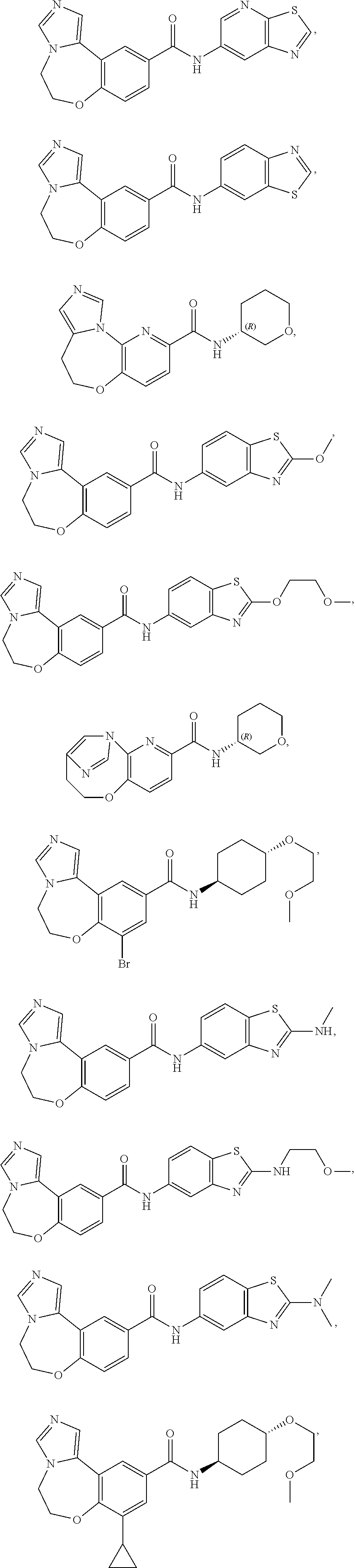 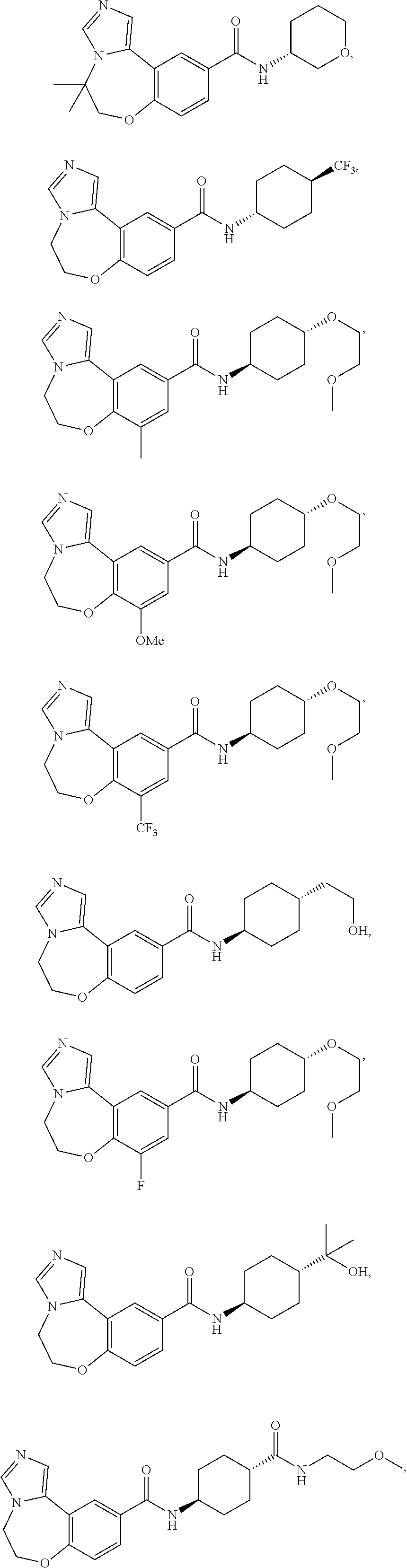 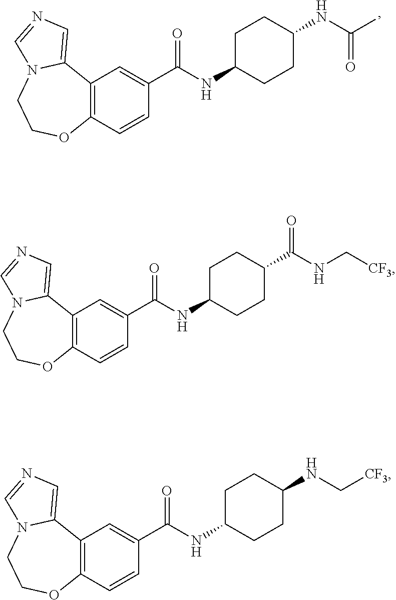 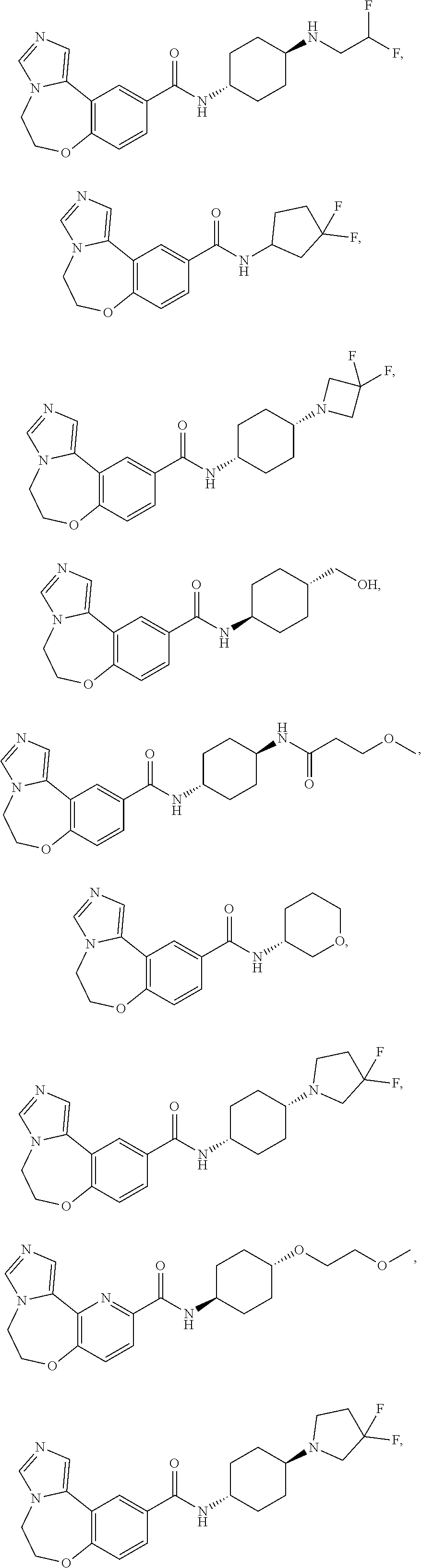 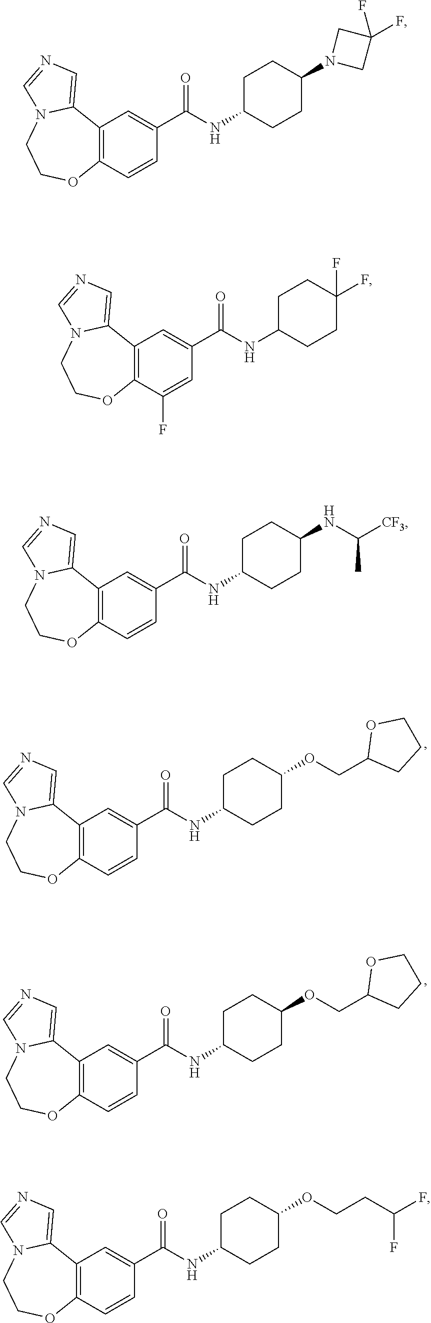 [SMILES: O=C(N[C@H]1CC[C@H](N2CC(F)(F)C2)CC1)c1ccc2c(c1)-c1cncn1CCO2 ; O=C(NC1CCC(F)(F)CC1)c1cc(F)c2c(c1)-c1cncn1CCO2 ; C[C@@H](N[C@H]1CC[C@H](NC(=O)c2ccc3c(c2)-c2cncn2CCO3)CC1)C(F)(F)F ; O=C(N[C@H]1CC[C@@H](OCC2CCCO2)CC1)c1ccc2c(c1)-c1cncn1CCO2 ; O=C(N[C@H]1CC[C@H](OCC2CCCO2)CC1)c1ccc2c(c1)-c1cncn1CCO2 ; O=C(N[C@H]1CC[C@@H](OCCC(F)F)CC1)c1ccc2c(c1)-c1cncn1CCO2] 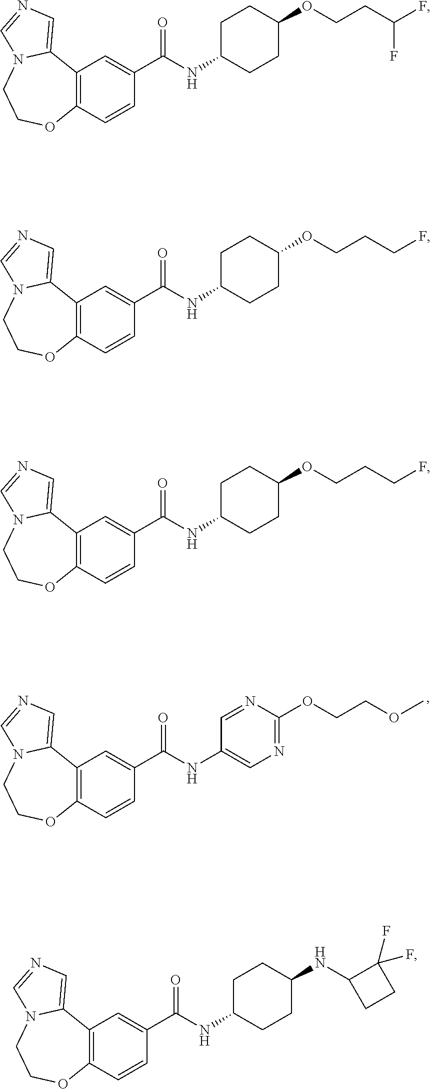 [SMILES: O=C(N[C@H]1CC[C@H](OCCC(F)F)CC1)c1ccc2c(c1)-c1cncn1CCO2 ; O=C(N[C@H]1CC[C@@H](OCCCF)CC1)c1ccc2c(c1)-c1cncn1CCO2 ; O=C(N[C@H]1CC[C@H](OCCCF)CC1)c1ccc2c(c1)-c1cncn1CCO2 ; COCCOc1ncc(NC(=O)c2ccc3c(c2)-c2cncn2CCO3)cn1 ; O=C(N[C@H]1CC[C@H](NC2CCC2(F)F)CC1)c1ccc2c(c1)-c1cncn1CCO2] 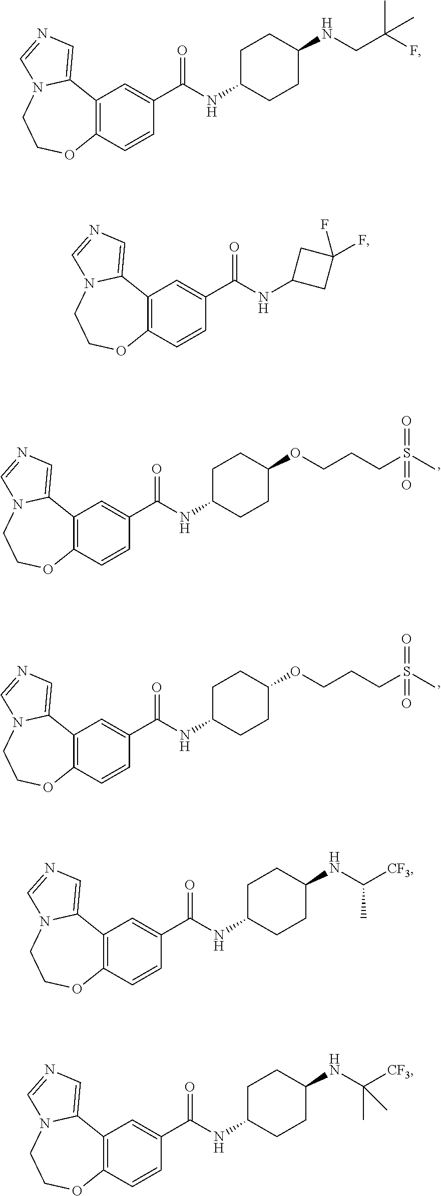 [SMILES: CC(C)(F)CN[C@H]1CC[C@H](NC(=O)c2ccc3c(c2)-c2cncn2CCO3)CC1 ; O=C(NC1CC(F)(F)C1)c1ccc2c(c1)-c1cncn1CCO2 ; CS(=O)(=O)CCCO[C@H]1CC[C@H](NC(=O)c2ccc3c(c2)-c2cncn2CCO3)CC1 ; CS(=O)(=O)CCCO[C@H]1CC[C@@H](NC(=O)c2ccc3c(c2)-c2cncn2CCO3)CC1 ; C[C@H](N[C@H]1CC[C@H](NC(=O)c2ccc3c(c2)-c2cncn2CCO3)CC1)C(F)(F)F ; CC(C)(N[C@H]1CC[C@H](NC(=O)c2ccc3c(c2)-c2cncn2CCO3)CC1)C(F)(F)F] 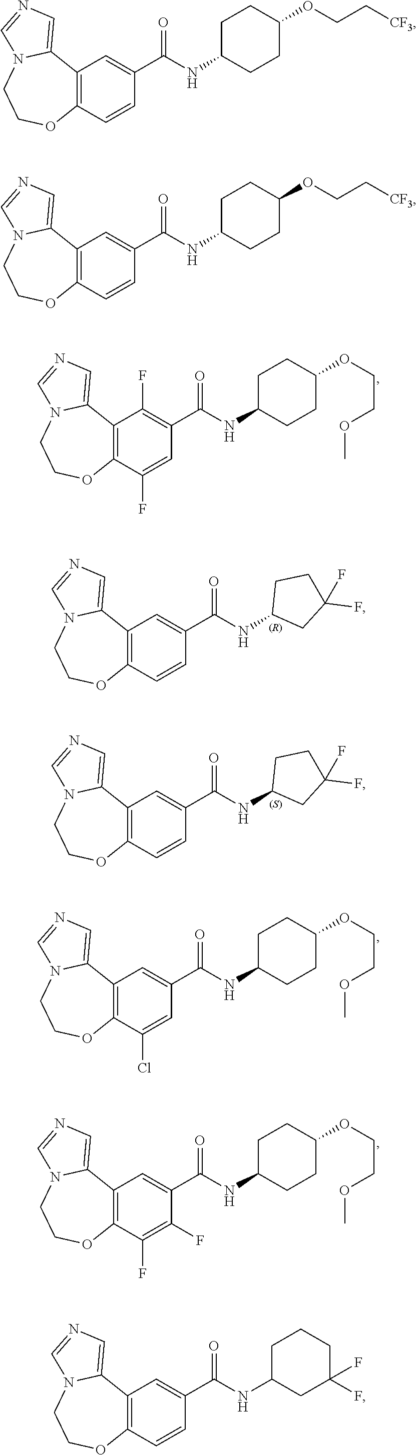 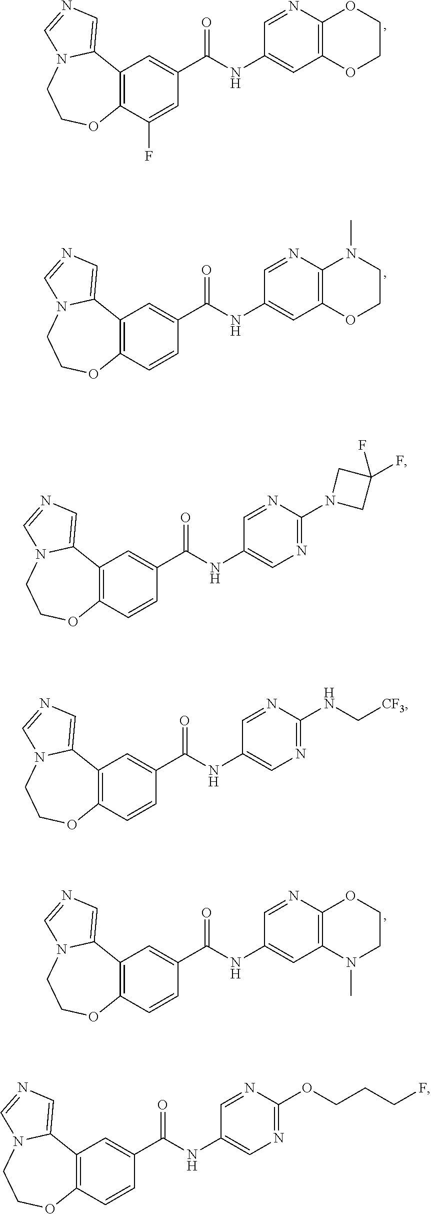 [SMILES: O=C(Nc1cnc2c(c1)OCCO2)c1cc(F)c2c(c1)-c1cncn1CCO2 ; CN1CCOc2cc(NC(=O)c3ccc4c(c3)-c3cncn3CCO4)cnc21 ; O=C(Nc1cnc(N2CC(F)(F)C2)nc1)c1ccc2c(c1)-c1cncn1CCO2 ; O=C(Nc1cnc(NCC(F)(F)F)nc1)c1ccc2c(c1)-c1cncn1CCO2 ; CN1CCOc2ncc(NC(=O)c3ccc4c(c3)-c3cncn3CCO4)cc21 ; O=C(Nc1cnc(OCCCF)nc1)c1ccc2c(c1)-c1cncn1CCO2] 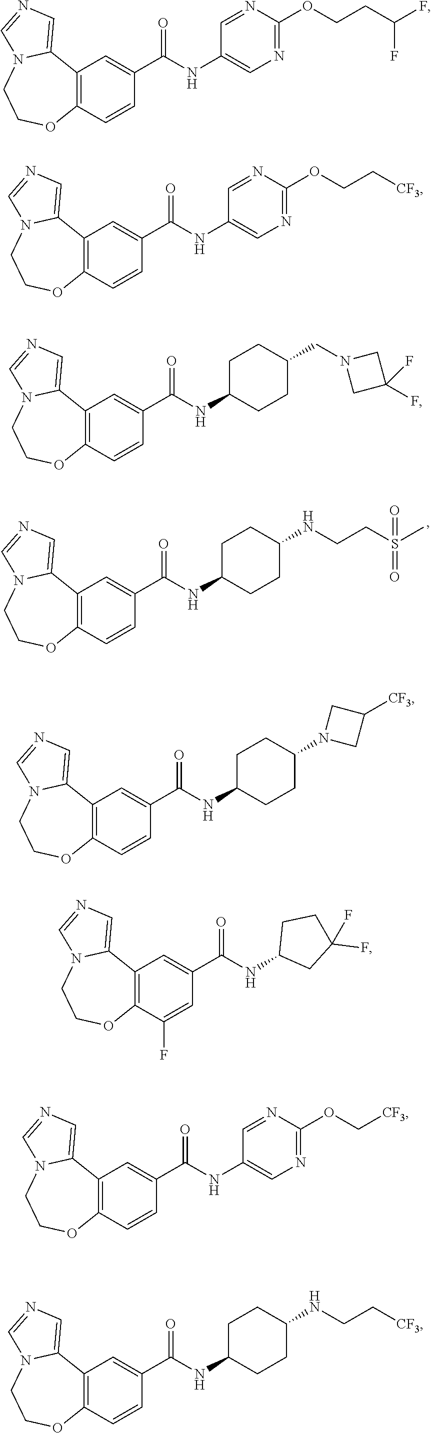 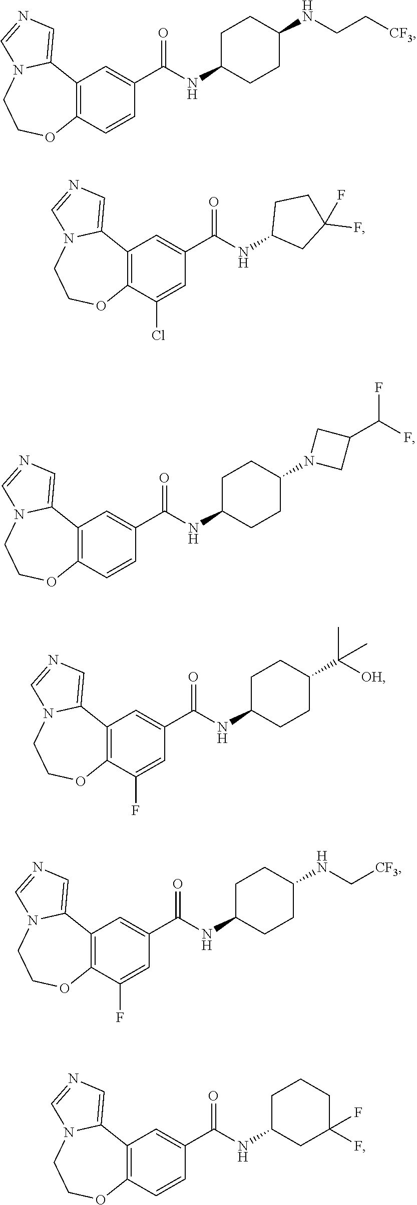 [SMILES: O=C(N[C@H]1CC[C@@H](NCCC(F)(F)F)CC1)c1ccc2c(c1)-c1cncn1CCO2 ; O=C(N[C@@H]1CCC(F)(F)C1)c1cc(Cl)c2c(c1)-c1cncn1CCO2 ; O=C(N[C@H]1CC[C@H](N2CC(C(F)F)C2)CC1)c1ccc2c(c1)-c1cncn1CCO2 ; CC(C)(O)[C@H]1CC[C@H](NC(=O)c2cc(F)c3c(c2)-c2cncn2CCO3)CC1 ; O=C(N[C@H]1CC[C@H](NCC(F)(F)F)CC1)c1cc(F)c2c(c1)-c1cncn1CCO2 ; O=C(N[C@@H]1CCCC(F)(F)C1)c1ccc2c(c1)-c1cncn1CCO2] 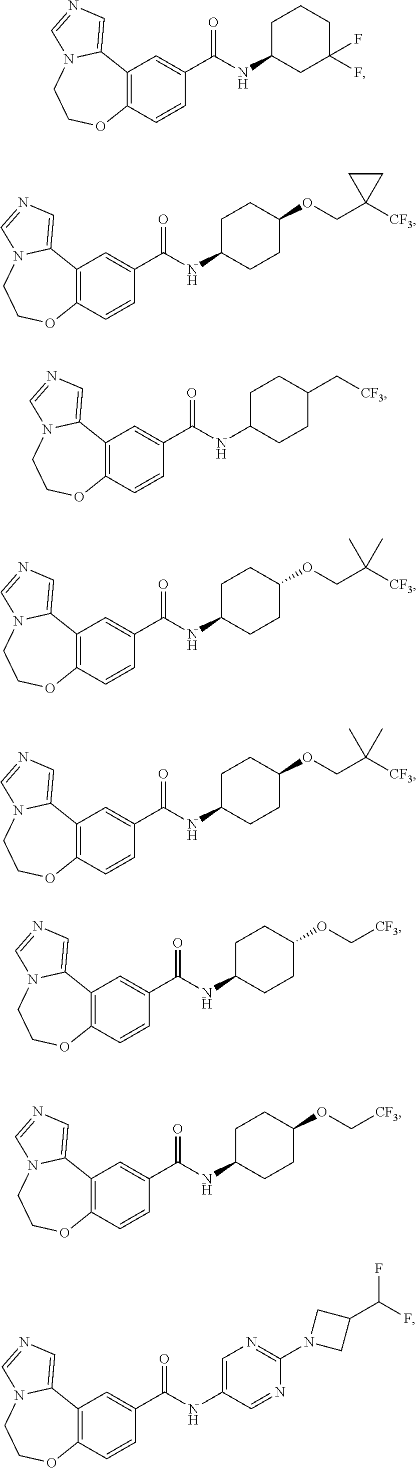 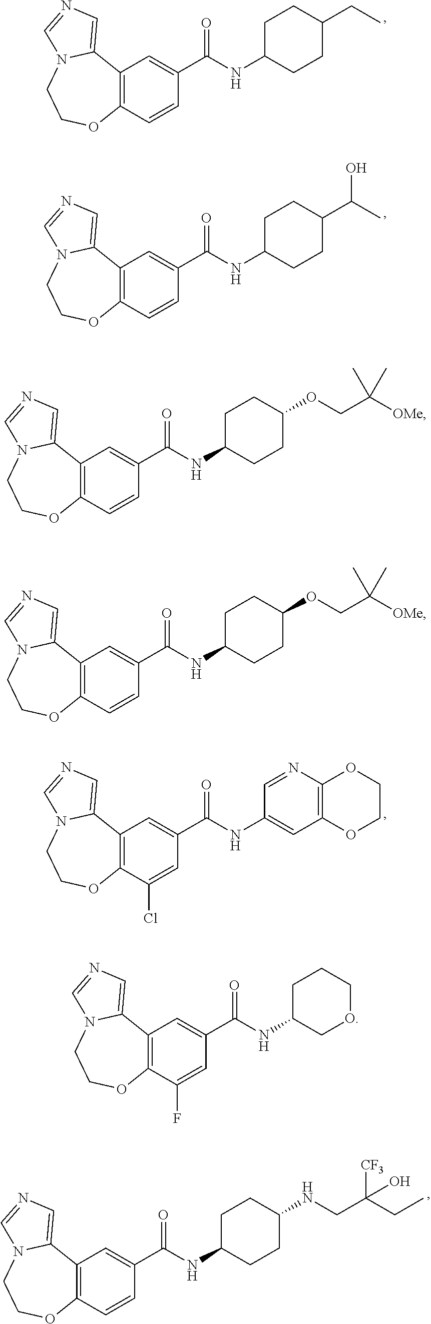 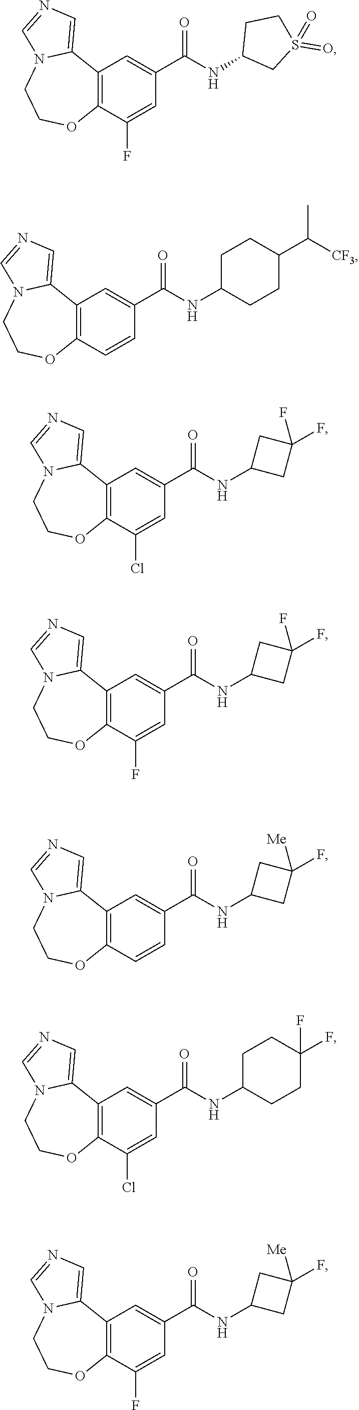 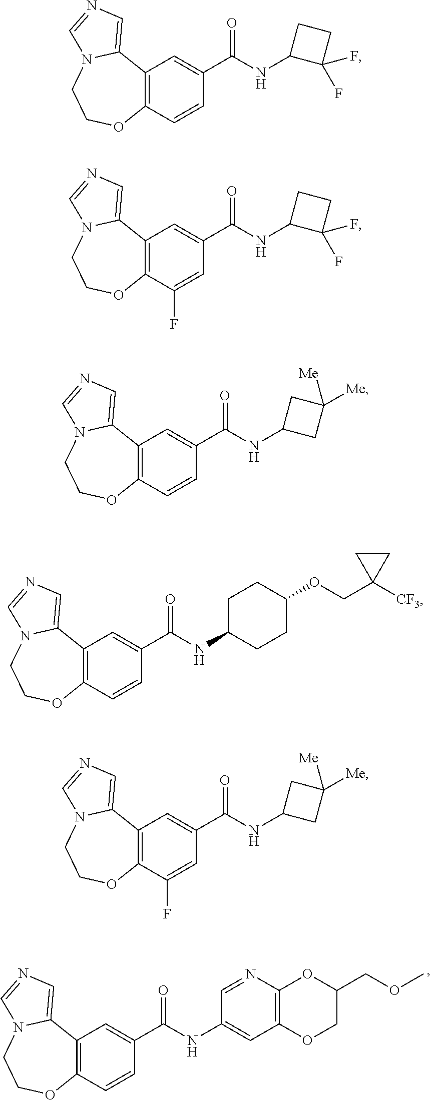 [SMILES: O=C(NC1CCC1(F)F)c1ccc2c(c1)-c1cncn1CCO2 ; O=C(NC1CCC1(F)F)c1cc(F)c2c(c1)-c1cncn1CCO2 ; CC1(C)CC(NC(=O)c2ccc3c(c2)-c2cncn2CCO3)C1 ; O=C(N[C@H]1CC[C@H](OCC2(C(F)(F)F)CC2)CC1)c1ccc2c(c1)-c1cncn1CCO2 ; CC1(C)CC(NC(=O)c2cc(F)c3c(c2)-c2cncn2CCO3)C1 ; COCC1COc2cc(NC(=O)c3ccc4c(c3)-c3cncn3CCO4)cnc2O1] 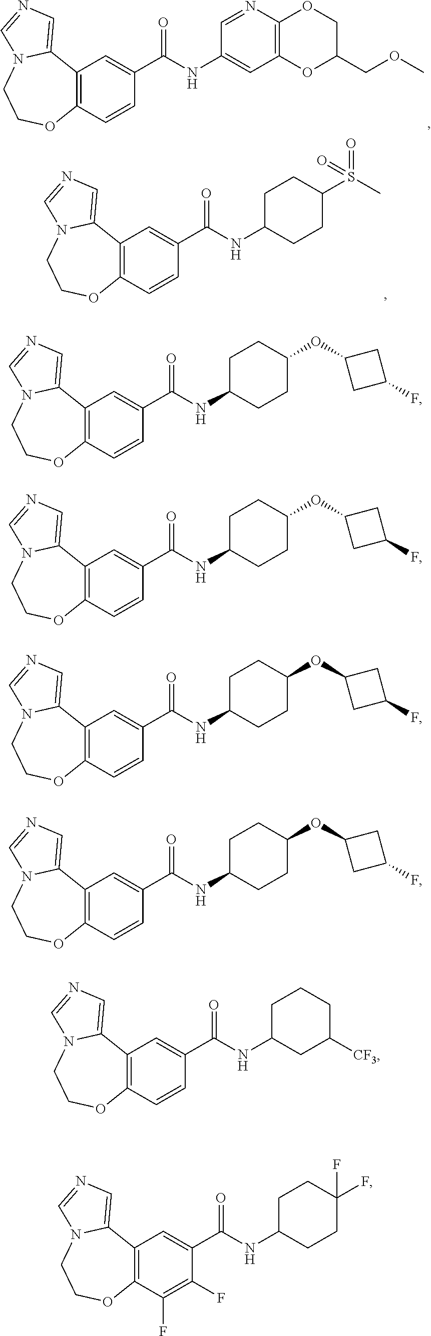 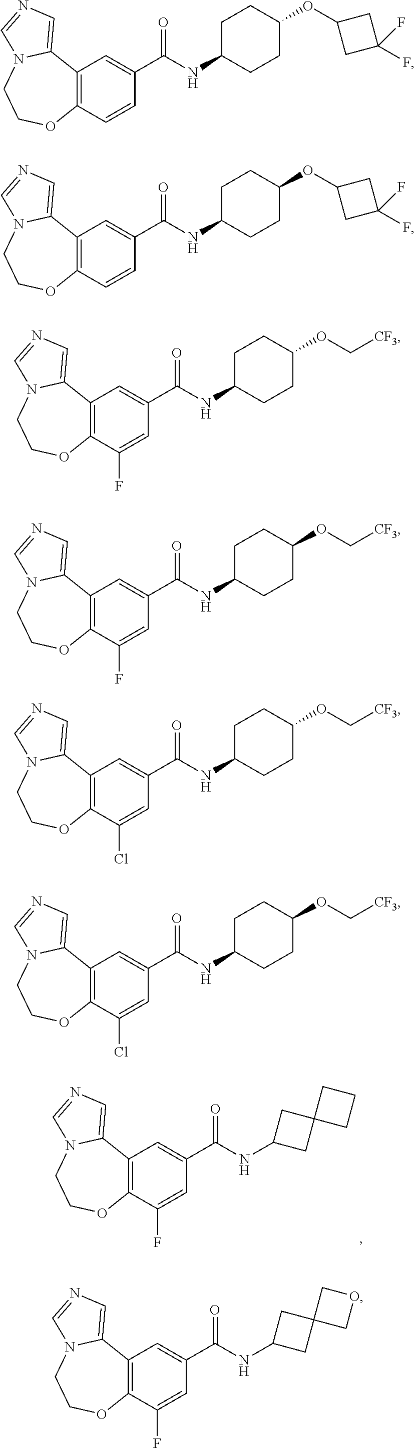 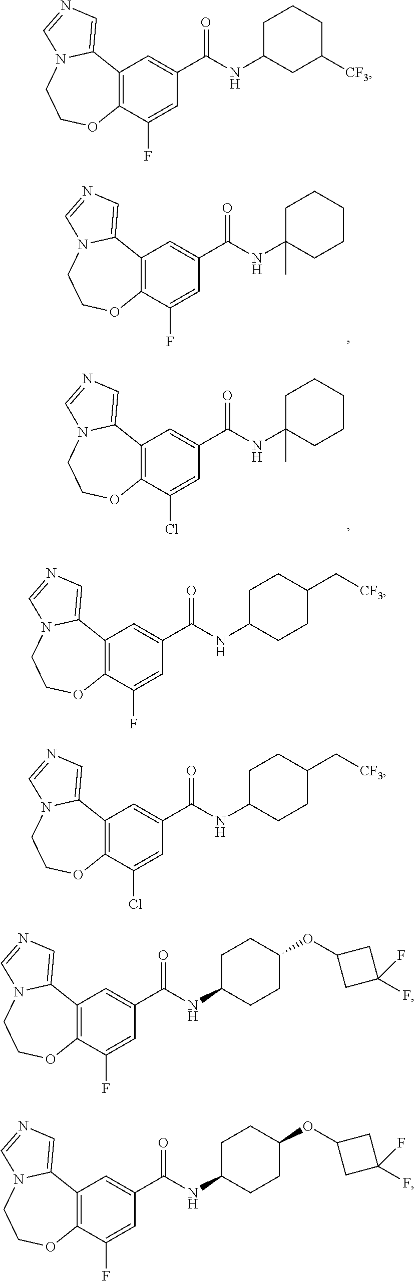 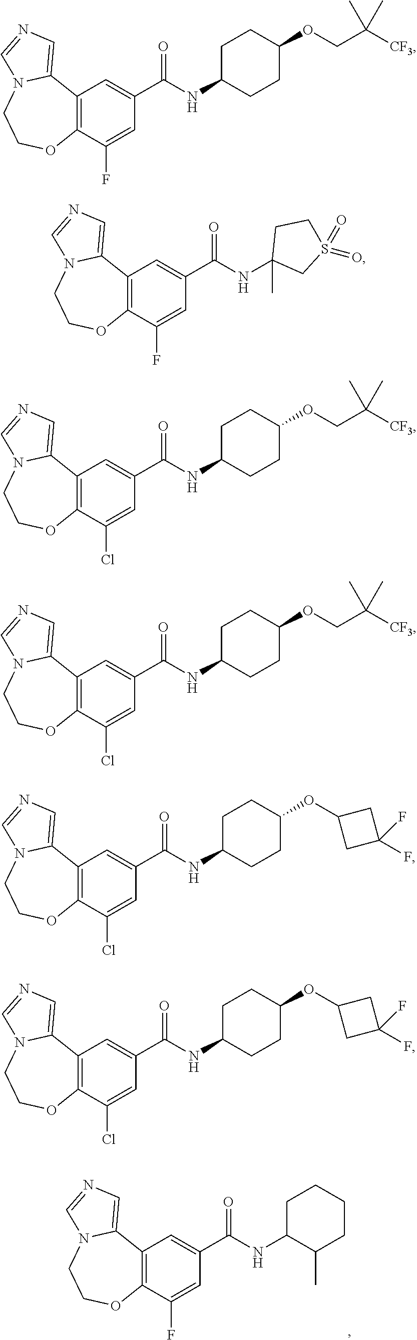 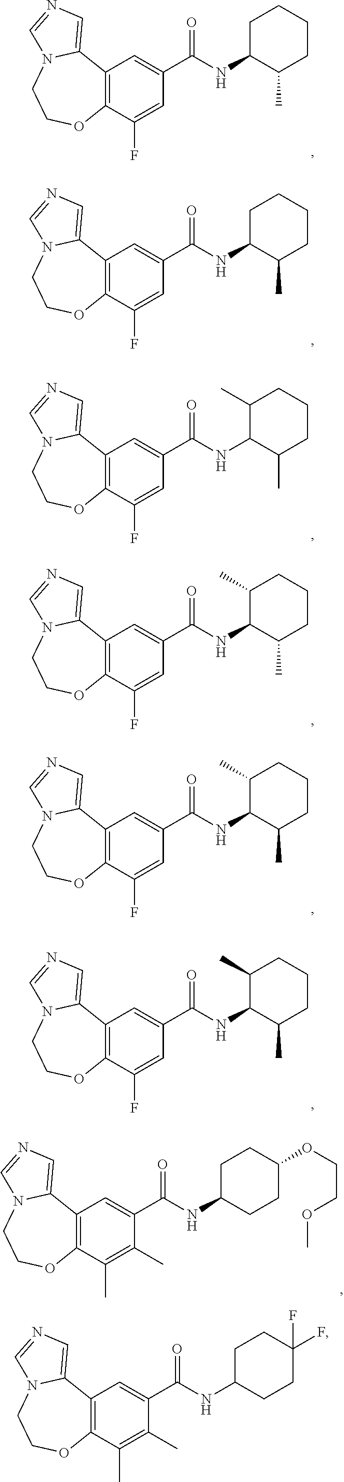 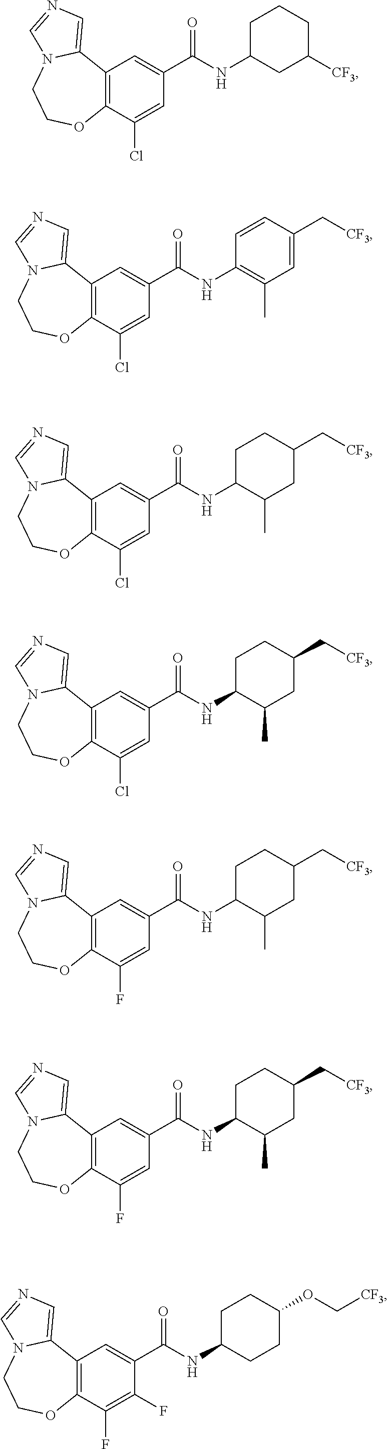 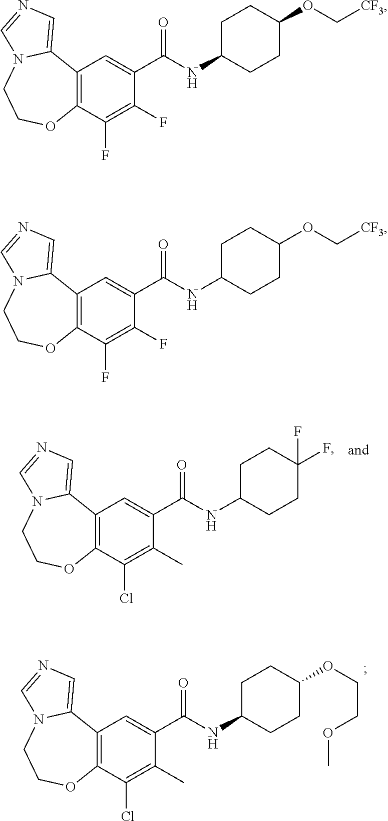 [SMILES: O=C(N[C@H]1CC[C@@H](OCC(F)(F)F)CC1)c1cc2c(c(F)c1F)OCCn1cncc1-2 ; O=C(NC1CCC(OCC(F)(F)F)CC1)c1cc2c(c(F)c1F)OCCn1cncc1-2 ; Cc1c(C(=O)NC2CCC(F)(F)CC2)cc2c(c1Cl)OCCn1cncc1-2 ; COCCO[C@H]1CC[C@H](NC(=O)c2cc3c(c(Cl)c2C)OCCn2cncc2-3)CC1]
or a pharmaceutically acceptable salt or solvate thereof.

138. A pharmaceutical composition comprising: a compound or salt of claim 61 and at least one pharmaceutically acceptable excipient.

139. (canceled)

140. A method of treating a disease or disorder, the method comprising administering to a subject in need thereof a therapeutically effective amount of a compound or salt of claim 61; wherein the method leads to inhibition of CD38 in the subject; and wherein the disease or disorder is selected from: a neurodegenerative disease, type I diabetes, insulin resistance, Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra, Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, fibrosis, pancreatitis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia-telangiectasia, rheumatoid arthritis, lupus, alcohol intolerance, hyperphosphatemia, acute lung injury, and ARDS.

141. (canceled)

142. (canceled)

143. (canceled)

144. The method of claim 140, wherein the disease or disorder is selected from a neurodegenerative disease, NASH, fibrosis, cardiac hypertrophy, muscular dystrophy, and obesity.

145. (canceled)

146. (canceled)

147. (canceled)

148. (canceled)

149. (canceled)

150. (canceled)

151. A method of modulating CD38 in a subject, comprising administering to the subject a compound or salt of claim 61.

152. (canceled)
